(19) United States
(12) Patent Application Publication
(10) Pub. No.: US 2021/0147550 A1
(43) Pub. Date: May 20, 2021

(54) ANTIGEN-BINDING PROTEINS TARGETING SHARED ANTIGENS

(71) Applicants: Joshua Michael FRANCIS, Emeryville, CA (US); Gijsbert Marnix GROTENBREG, Emeryville, CA (US); Gritstone Oncology, Inc., Emeryville, CA (US)

(72) Inventors: Karin Jooss, Emeryville, CA (US); Wade Blair, Emeryville, CA (US); Brendan Bulik-Sullivan, Emeryville, CA (US); Jennifer Busby, Emeryville, CA (US); Michele Anne Busby, Emeryville, CA (US); Joshua Michael Francis, Emeryville, CA (US); Gijsbert Marnix Grotenbreg, Emeryville, CA (US); Mojca Skoberne, Emeryville, CA (US); Roman Yelensky, Emeryville, CA (US)

(21) Appl. No.: 16/639,073

(22) PCT Filed: Aug. 17, 2018
(86) PCT No.: PCT/US18/46997

§ 371 Date: Feb. 13, 2020

Related U.S. Application Data
(60) Provisional application No. 62/547,146, filed on Aug. 18, 2017.
(60) Provisional application No. 62/581,368, filed on Nov. 3, 2017.

(51) Int. Cl. C07K 16/28; C07K 14/725; G01N 33/574

(52) U.S. Cl. CPC C07K 16/2833; C07K 14/7051; G01N 33/574; C07K 2317/32; G01N 2500/10; C07K 2317/55; C07K 2317/622; C07K 2317/92; G01N 2333/70539; C07K 2317/34

(57) ABSTRACT

Provided herein are HLA-PEPTIDE targets and antigen binding proteins that bind HLA-PEPTIDE targets. Also disclosed are methods for identifying the HLA-PEPTIDE targets as well as identifying one or more antigen binding proteins that bind a given HLA-PEPTIDE target.

### CROSS REFERENCE TO RELATED APPLICATIONS

This application claims the benefit of U.S. Provisional Application No. 62/547,146, filed on Aug. 18, 2017, and of U.S. Provisional Application No. 62/581,368, filed on Nov. 3, 2017, which applications are hereby incorporated in entirety by reference for all purposes.

### SEQUENCE LISTING

The instant application contains a Sequence Listing which has been filed electronically in ASCII format and is hereby incorporated by reference in its entirety. Said ASCII copy, created on Aug. 16, 2018, is named 40698PCT_CRF_sequencelisting.txt and is 1,591,443 bytes in size.

### BACKGROUND

The immune system employs two types of immune responses to provide antigen specific protection from pathogens; humoral immune responses, and cellular immune responses, which involve specific recognition of pathogen antigens via B lymphocytes and T lymphocytes, respectively.

T lymphocytes, by virtue of being the antigen specific effectors of cellular immunity, play a central role in the body's defense against diseases mediated by intracellular pathogens, such as viruses, intracellular bacteria, mycoplasmas, and intracellular parasites, by directly cytolysing cells infected by such pathogens. The specificity of T lymphocyte responses is conferred by, and activated through T-cell receptors (TCRs). T-cell receptors are antigen specific receptors clonally distributed on individual T lymphocytes whose repertoire of antigenic specificity is generated via somatic gene rearrangement mechanisms analogous to those involved in generating the antibody gene repertoire. T-cell receptors include a heterodimer of transmembrane molecules, the main type being composed of an alpha-beta polypeptide dimer and a smaller subset of a gamma-delta polypeptide dimer. T lymphocyte receptor subunits comprise a variable and constant region similar to immunoglobulins in the extracellular domain, a short hinge region with cysteine that promotes alpha and beta chain pairing, a transmembrane and a short cytoplasmic region. Signal transduction triggered by TCRs is indirectly mediated via CD3-zeta, an associated multi-subunit complex comprising signal transducing subunits.

T lymphocyte receptors do not generally recognize native antigens but rather recognize cell-surface displayed complexes comprising an intracellularly processed fragment of an antigen in association with a major histocompatibility complex (MHC) for presentation of peptide antigens. Major histocompatibility complex genes are highly polymorphic across species populations, comprising multiple common alleles for each individual gene.

Major histocompatibility complex class I molecules are expressed on the surface of virtually all nucleated cells in the body and are dimeric molecules comprising a transmembrane heavy chain, comprising the peptide antigen binding cleft, and a smaller extracellular chain termed beta2-microglobulin. MHC class I molecules present peptides derived from the degradation of cytosolic proteins by the proteasome, a multi-unit structure in the cytoplasm, (Niedermann G, 2002. Curr Top Microbiol Immunol. 268:91-136; for processing of bacterial antigens, refer to Wick M J, and Ljunggren H G, 1999. Immunol Rev. 172:153-62). Cleaved peptides are transported into the lumen of the endoplasmic reticulum (ER) by TAP where they are bound to the groove of the assembled class I molecule, and the resultant MHC/peptide complex is transported to the cell membrane to enable antigen presentation to T lymphocytes (Yewdell J W., 2001. Trends Cell Biol. 11:294-7; Yewdell J W. and Bennink J R., 2001. Curr Opin Immunol. 13:13-8). Alternatively, cleaved peptides can be loaded onto MHC class I molecules in TAP-independent manner and can also present extracellularly-derived proteins through a process of cross-presentation. As such, a given MHC/peptide complex presents a novel protein structure on the cell surface that can be targeted by a novel antigen-binding protein (e.g., antibodies or TCRs) once the identity of the complex's structure (peptide sequence and MHC subtype) is determined.

Tumor cells can express antigens and may display such antigens on the surface of the tumor cell. Such tumor-associated antigens can be used for development of novel immunotherapeutic reagents for the specific targeting of tumor cells. For example, tumor-associated antigens can be used to identify therapeutic antigen binding proteins, e.g., TCRs, antibodies, or antigen-binding fragments. Such tumor-associated antigens may also be utilized in pharmaceutical compositions, e.g., vaccines.

### SUMMARY

In an aspect, provided herein is an isolated antigen binding protein (ABP) that specifically binds to a human leukocyte antigen (HLA)-PEPTIDE target, wherein the HLA-PEPTIDE target comprises an HLA-restricted peptide complexed with an HLA Class I molecule, wherein the HLA-restricted peptide is located in the peptide binding groove of an al/2 heterodimer portion of the HLA Class I molecule, and wherein: the HLA Class I molecule is HLA subtype A*02:01 and the HLA-restricted peptide comprises the sequence LLASSILCA, the HLA Class I molecule is HLA subtype A*01:01 and the HLA-restricted peptide comprises the sequence EVDPIGHLY, the HLA Class I molecule is HLA subtype B*44:02 and the HLA-restricted peptide comprises the sequence GEMSSNSTAL, the HLA Class I molecule is HLA subtype A*02:01 and the HLA-restricted peptide comprises the sequence GVYDGEEHSV, the HLA Class I molecule is HLA subtype *01:01 and the HLA-restricted peptide comprises the sequence EVDPIGHVY, or the HLA Class I molecule is HLA subtype HLA-A*01:01 and the HLA-restricted peptide comprises the sequence NTDNNLAVY

In some embodiments, the HLA Class I molecule is HLA subtype A*02:01 and the HLA-restricted peptide consists of the sequence LLASSILCA, the HLA Class I molecule is HLA subtype A*01:01 and the HLA-restricted peptide consists of the sequence EVDPIGHLY, the HLA Class I molecule is HLA subtype B*44:02 and the HLA-restricted peptide consists of the sequence GEMSSNSTAL, the HLA Class I molecule is HLA subtype A*02:01 and the HLA-restricted peptide consists of the sequence GVYDGEEHSV, the HLA Class I molecule is HLA subtype *01:01 and the HLA-restricted peptide consists of the sequence EVDPIGHVY, or the HLA Class I molecule is HLA subtype HLA-A*01:01 and the HLA-restricted peptide consists of the sequence NTDNNLAVY

In some embodiments, the HLA-restricted peptide is between about 5-15 amino acids in length. In some embodiments, the HLA-restricted peptide is between about 8-12 amino acids in length.

In an aspect, the ABP comprises an antibody or antigen-binding fragment thereof.

In some embodiments, the HLA Class I molecule is HLA subtype A*02:01 and the HLA-restricted peptide comprises the sequence LLASSILCA. In some embodiments, the ABP comprises a CDR-H3 comprising a sequence set forth in any one of SEQ ID NOS: 3025-3032. In some embodiments, the ABP comprises a CDR-L3 comprising a sequence set forth in any one of SEQ ID NOS: 3043-3050 In some embodiments, the ABP comprises the CDR-H3 and the CDR-L3 from the scFv designated G7R3-P1C6, G7R3-P1G10, 1-G7R3-P1B4, 2-G7R4-P2C2, 3-G7R4-P1A3, 4-G7R4-B5-P2E9, 5-G7R4-B10-P1F8, or B7 (G7R3-P3A9). In some embodiments, the ABP comprises all three heavy chain CDRs and all three light chain CDRs from the scFv designated G7R3-P1C6, G7R3-P1G10, 1-G7R3-P1B4, 2-G7R4-P2C2, 3-G7R4-P1A3, 4-G7R4-B5-P2E9, 5-G7R4-B10-P1F8, or B7 (G7R3-P3A9). In some embodiments, the ABP comprises a VH sequence selected from SEQ ID NO: 2994-3001. In some embodiments, the ABP comprises a VL sequence selected from SEQ ID NO: 3002-3009. In some embodiments, the ABP comprises the VH sequence and VL sequence from the scFv designated G7R3-P1C6, G7R3-P1G10, 1-G7R3-P1B4, 2-G7R4-P2C2, 3-G7R4-P1A3, 4-G7R4-B5-P2E9, 5-G7R4-B10-P1F8, or B7 (G7R3-P3A9). In some embodiments, the ABP binds to the HLA-PEPTIDE target via any one or more of residues 1-5 of the restricted peptide LLASSILCA.

In some embodiments, the HLA Class I molecule is HLA subtype HLA-A*01:01 and the HLA-restricted peptide comprises the sequence NTDNNLAVY In some embodiments, the ABP comprises a CDR-H3 comprising a sequence set forth in any one of SEQ ID NOS: 2902-2933. In some embodiments, the ABP comprises a CDR-L3 comprising a sequence set forth in any one of SEQ ID NOS: 2971-2993. In some embodiments, the ABP comprises the CDR-H3 and the CDR-L3 from the scFv designated G2-P2E07, G2-P2E03, G2-P2A11, G2-P2C06, G2-P1G01, G2-P1C02, G2-P1H01, G2-P1B12, G2-P1B06, G2-P2H10, G2-P1H10, G2-P2C11, G2-P1C09, G2-P1A10, G2-P1B10, G2-P1D07, G2-P1E05, G2-P1D03, G2-P1G12, G2-P2H11, G2-P1C03, G2-P1G07, G2-P1F12, G2-P1G03, G2-P2B08, G2-P2A10, G2-P2D04, G2-P1C06, G2-P2A09, G2-P1B08, G2-P1E03, G2-P2A03, G2-P2F01, G2-P1H11, or G2-P1D06: In some embodiments, the ABP comprises all three heavy chain CDRs and all three light chain CDRs from the scFv designated G2-P2E07, G2-P2E03, G2-P2A11, G2-P2C06, G2-P1G01, G2-P1C02, G2-P1H01, G2-P1B12, G2-P1B06, G2-P2H10, G2-P1H10, G2-P2C11, G2-P1C09, G2-P1A10, G2-P1B10, G2-P1D07, G2-P1E05, G2-P1D03, G2-P1G12, G2-P2H11, G2-P1C03, G2-P1G07, G2-P1F12, G2-P1G03, G2-P2B08, G2-P2A10, G2-P2D04, G2-P1C06, G2-P2A09, G2-P1B08, G2-P1E03, G2-P2A03, G2-P2F01, G2-P1H11, or G2-P1D06: In some embodiments, the ABP comprises a VH sequence selected from SEQ ID NO: 2781-2815. In some embodiments, the ABP comprises a VL sequence selected from SEQ ID NO: 2816-2850. In some embodiments, the ABP comprises the VH sequence and VL sequence from the scFv designated G2-P2E07, G2-P2E03, G2-P2A11, G2-P2C06, G2-P1G01, G2-P1C02, G2-P1H01, G2-P1B12, G2-P1B06, G2-P2H10, G2-P1H10, G2-P2C11, G2-P1C09, G2-P1A10, G2-P1B10, G2-P1D07, G2-P1E05, G2-P1D03, G2-P1G12, G2-P2H11, G2-P1C03, G2-P1G07, G2-P1F12, G2-P1G03, G2-P2B08, G2-P2A10, G2-P2D04, G2-P1C06, G2-P2A09, G2-P1B08, G2-P1E03, G2-P2A03, G2-P2F01, G2-P1H11, or G2-P1D06. In some embodiments, the ABP binds to the HLA-PEPTIDE target via residues 6-9 of the restricted peptide NTDNNLAVY and via residues 157-160 of the HLA subtype allele A*0101. In some embodiments, the ABP binds to the HLA-PEPTIDE target via residues 3-8 of the restricted peptide NTDNNLAVY.

In another aspect, the ABP comprises a T cell receptor (TCR) or an antigen-binding portion thereof. In some embodiments, the TCR or antigen-binding portion thereof comprises a TCR variable region. In some embodiments, the TCR or antigen-binding portion thereof comprises one or more TCR complementarity determining regions (CDRs). In some embodiments, the TCR comprises an alpha chain and a beta chain. In some embodiments, the TCR comprises a gamma chain and a delta chain. In some embodiments, the antigen binding protein is a portion of a chimeric antigen receptor (CAR) comprising: an extracellular portion comprising the antigen binding protein; and an intracellular signaling domain. In some embodiments, the antigen binding protein comprises an scFv and the intracellular signaling domain comprises an ITAM. In some embodiments, the intracellular signaling domain comprises a signaling domain of a zeta chain of a CD3-zeta (CD3) chain. In some embodiments, the ABP further comprises a transmembrane domain linking the extracellular domain and the intracellular signaling domain. In some embodiments, the transmembrane domain comprises a transmembrane portion of CD28. In some embodiments, the ABP further comprises an intracellular signaling domain of a T cell costimulatory molecule. In some embodiments, the T cell costimulatory molecule is CD28, 4-1BB, OX-40, ICOS, or any combination thereof.

In some embodiments, the HLA Class I molecule is HLA subtype A*02:01 and the HLA-restricted peptide comprises the sequence LLASSILCA. In some embodiments, the ABP comprises a TCR alpha CDR3 sequence that is SEQ ID NO: 4277, 4278, 4279, 4280, or 4281. In some embodiments, the ABP comprises a TCR beta CDR3 sequence that is any one of SEQ ID NOS 4291-4295. In some embodiments, the ABP comprises an alpha CDR3 and a beta CDR3 sequence from any one of TCR clonotype ID #s: TCR19, TCR21, TCR22, TCR18, or TCR23. In some embodiments, the ABP comprises a TCR alpha variable (TRAV) amino acid sequence, a TCR alpha joining (TRAJ) amino acid sequence, a TCR beta variable (TRBV) amino acid sequence, a TCR beta diversity (TRBD) amino acid sequence, and a TCR beta joining (TRBJ) amino acid sequence, wherein each of the TRAV, TRAJ, TRBV, TRBD, and TRBJ amino acid sequences are at least 95%, 96%, 97%, 98%, 99%, or 100% identical to the corresponding TRAV, TRAJ, TRBV, TRBD, and TRBJ amino acid sequences for any one of the TCR clonotypes selected from TCR clonotype ID #s: TCR19, TCR21, TCR22, TCR18, and TCR23. In some embodiments, the ABP comprises a TCR alpha constant (TRAC) amino acid sequence. In some embodiments, the ABP comprises a TCR beta constant (TRBC) amino acid sequence. In some embodiments, the ABP comprises a TCR alpha VJ sequence having at least 95%, 96%, 97%, 98%, 99%, or 100% identity to any one of SEQ ID NOS 4306-4310. In some embodiments, the ABP comprises a TCR beta V(D)J sequence having at least 95%, 96%, 97%, 98%, 99%, or 100% identity to any one of SEQ ID NOS 4321-4325. In some embodiments, the ABP comprises a TCR alpha VJ amino acid sequence and a TCR beta V(D)J amino acid sequence, wherein each of the TCR alpha VJ and the TCR beta V(D)J amino acid sequences are at least 95%, 96%, 97%, 98%, 99%, or 100% identical to the corresponding TCR alpha VJ and TCR beta V(D)J amino acid sequences for any one of the TCR clonotypes selected from TCR clonotype ID #s: TCR19, TCR21, TCR22, TCR18, and TCR23.

In some embodiments, the HLA Class I molecule is HLA subtype A*01:01 and the HLA-restricted peptide comprises the sequence EVDPIGHLY. In some embodiments, the ABP comprises a TCR alpha CDR3 sequence that is any one of SEQ ID NOS: 4273-4276 or 3052-3350. In some embodiments, the ABP comprises a TCR beta CDR3 sequence that is any one of SEQ ID NOS: 4287-4290 or 3351-3655. In some embodiments, the ABP comprises an alpha CDR3 and a beta CDR3 sequence from any one of TCR ID #s: TCR101-TCR469, TCR2, TCR4, TCR53, TCR54, or TCR101-TCR469. In some embodiments, the ABP comprises a TCR alpha variable (TRAV) amino acid sequence, a TCR alpha joining (TRAJ) amino acid sequence, a TCR beta variable (TRBV) amino acid sequence, a TCR beta diversity (TRBD) amino acid sequence, and a TCR beta joining (TRBJ) amino acid sequence, wherein each of the TRAV, TRAJ, TRBV, TRBD, and TRBJ amino acid sequences are at least 95%, 96%, 97%, 98%, 99%, or 100% identical to the corresponding TRAV, TRAJ, TRBV, TRBD, and TRBJ amino acid sequences for any one of the TCR clonotypes selected from TCR ID #s: TCR101-TCR469, TCR2, TCR4, TCR53, TCR54, or TCR101-TCR469. In some embodiments, the ABP comprises a TCR alpha constant (TRAC) amino acid sequence. In some embodiments, the ABP comprises a TCR beta constant (TRBC) amino acid sequence. In some embodiments, the ABP comprises a TCR alpha VJ sequence having at least 95%, 96%, 97%, 98%, 99%, or 100% identity to any one of SEQ ID NOS: 3656-3961 or 4302-4305. In some embodiments, the ABP comprises a TCR beta V(D)J sequence having at least 95%, 96%, 97%, 98%, 99%, or 100% identity to any one of SEQ ID NOS: 3962-4269 or 4317-4320. In some embodiments, the ABP comprises a TCR alpha VJ amino acid sequence and a TCR beta V(D)J amino acid sequence, wherein each of the TCR alpha VJ and the TCR beta V(D)J amino acid sequences are at least 95%, 96%, 97%, 98%, 99%, or 100% identical to the corresponding TCR alpha VJ and TCR beta V(D)J amino acid sequences for any one of the TCR clonotypes selected from TCR ID #s: TCR101-TCR469, TCR2, TCR4, TCR53, and TCR54.

In some embodiments, the HLA Class I molecule is HLA subtype B*44:02 and the HLA-restricted peptide comprises the sequence GEMSSNSTAL. In some embodiments, the ABP comprises a TCR alpha CDR3 sequence that is be any one of SEQ ID NOS 4284-4286 or 3138. In some embodiments, the ABP comprises a TCR beta CDR3 sequence that is any one of SEQ ID NOS 4298-4301. In some embodiments, the ABP comprises an alpha CDR3 and a beta CDR3 sequence from any one of TCR ID #s: TCR29, TCR30, TCR32, or TCR33. In some embodiments, the ABP comprises a TCR alpha variable (TRAV) amino acid sequence, a TCR alpha joining (TRAJ) amino acid sequence, a TCR beta variable (TRBV) amino acid sequence, a TCR beta diversity (TRBD) amino acid sequence, and a TCR beta joining (TRBJ) amino acid sequence, wherein each of the TRAV, TRAJ, TRBV, TRBD, and TRBJ amino acid sequences are at least 95%, 96%, 97%, 98%, 99%, or 100% identical to the corresponding TRAV, TRAJ, TRBV, TRBD, and TRBJ amino acid sequences for any one of the TCR clonotypes selected from TCR ID #s: TCR29, TCR30, TCR32, or TCR33. In some embodiments, the ABP comprises a TCR alpha constant (TRAC) amino acid sequence. In some embodiments, the ABP comprises a TCR beta constant (TRBC) amino acid sequence. In some embodiments, the ABP comprises a TCR alpha VJ sequence having at least 95%, 96%, 97%, 98%, 99%, or 100% identity to any one of SEQ ID NOS: 4313-4316. In some embodiments, the ABP comprises a TCR beta V(D)J sequence having at least 95%, 96%, 97%, 98%, 99%, or 100% identity to any one of SEQ ID NOS: 4328-4331. In some embodiments, the ABP comprises a TCR alpha VJ amino acid sequence and a TCR beta V(D)J amino acid sequence, wherein each of the TCR alpha VJ and the TCR beta V(D)J amino acid sequences are at least 95%, 96%, 97%, 98%, 99%, or 100% identical to the corresponding TCR alpha VJ and TCR beta V(D)J amino acid sequences for any one of the TCR clonotypes selected from TCR ID #s: TCR29, TCR30, TCR32, or TCR33.

In some embodiments, the HLA Class I molecule is HLA subtype A*02:01 and the HLA-restricted peptide comprises the sequence GVYDGEEHSV In some embodiments, the ABP comprises a TCR alpha CDR3 sequence that is SEQ ID NO: 4282 or 4283. In some embodiments, the ABP comprises a TCR beta CDR3 sequence that is SEQ ID NO: 4296 or 4297. In some embodiments, the ABP comprises an alpha CDR3 and a beta CDR3 sequence from TCR clonotype ID #: TCR26 or TCR28. In some embodiments, the ABP comprises a TCR alpha variable (TRAV) amino acid sequence, a TCR alpha joining (TRAJ) amino acid sequence, a TCR beta variable (TRBV) amino acid sequence, a TCR beta diversity (TRBD) amino acid sequence, and a TCR beta joining (TRBJ) amino acid sequence, wherein each of the TRAV, TRAJ, TRBV, TRBD, and TRBJ amino acid sequences are at least 95%, 96%, 97%, 98%, 99%, or 100% identical to the corresponding TRAV, TRAJ, TRBV, TRBD, and TRBJ amino acid sequences for TCR ID #: TCR26 or TCR28. In some embodiments, the ABP comprises a TCR alpha constant (TRAC) amino acid sequence. In some embodiments, the ABP comprises a TCR beta constant (TRBC) amino acid sequence. In some embodiments, the ABP comprises a TCR alpha VJ sequence having at least 95%, 96%, 97%, 98%, 99%, or 100% identity to SEQ ID NO: 4311 or 4312. In some embodiments, the ABP comprises a TCR beta V(D)J sequence having at least 95%, 96%, 97%, 98%, 99%, or 100% identity to SEQ ID NO: 4326 or 4327. In some embodiments, the ABP comprises a TCR alpha VJ amino acid sequence and a TCR beta V(D)J amino acid sequence, wherein each of the TCR alpha VJ and the TCR beta V(D)J amino acid sequences are at least 95%, 96%, 97%, 98%, 99%, or 100% identical to the corresponding TCR alpha VJ and TCR beta V(D)J amino acid sequences for TCR ID #: TCR26 or TCR28.

In some embodiments, the HLA Class I molecule is HLA subtype HLA-A*01:01 and the HLA-restricted peptide comprises the sequence NTDNNLAVY In some embodiments, the HLA Class I molecule is HLA subtype HLA-A*03:01 and the HLA-restricted peptide comprises the sequence GVHGGILNK. In some embodiments, the HLA Class I molecule is HLA subtype HLA-A*01:01 and the HLA-restricted peptide comprises the sequence EVDPIGHVY

In another aspect, provided herein is an isolated antigen binding protein (ABP) that specifically binds to a human leukocyte antigen (HLA)-PEPTIDE target, wherein the HLA-PEPTIDE target comprises an HLA-restricted peptide complexed with an HLA Class I molecule, wherein the HLA-restricted peptide is located in in the peptide binding groove of an α1/α2 heterodimer portion of the HLA Class I molecule, and wherein the HLA-PEPTIDE target is selected from Table A. In some embodiments, the HLA-restricted peptide is not from a gene selected from WT1 or MART1. In some embodiments, the HLA-restricted peptide is between about 5-15 amino acids in length. In some embodiments, the HLA-restricted peptide is between about 8-12 amino acids in length. In some embodiments, the ABP comprises an antibody or antigen-binding fragment thereof.

In some embodiments of any of the antibodies or antigen binding fragments disclosed herein, the antigen binding protein is linked to a scaffold, optionally wherein the scaffold comprises serum albumin or Fc, optionally wherein Fc is human Fc and is an IgG (IgG1, IgG2, IgG3, IgG4), an IgA (IgA1, IgA2), an IgD, an IgE, or an IgM. In some embodiments of any of the antibodies or antigen binding fragments disclosed herein, the antigen binding protein is linked to a scaffold via a linker, optionally wherein the linker is a peptide linker, optionally wherein the peptide linker is a hinge region of a human antibody. In some embodiments of any of the antibodies or antigen binding fragments disclosed herein, the antigen binding protein comprises an Fv fragment, a Fab fragment, a F(ab′)2 fragment, a Fab′ fragment, an scFv fragment, an scFv-Fc fragment, and/or a single-domain antibody or antigen binding fragment thereof. In some embodiments of any of the antibodies or antigen binding fragments disclosed herein, the antigen binding protein comprises an scFv fragment. In some embodiments of any of the antibodies or antigen binding fragments disclosed herein, the antigen binding protein comprises one or more antibody complementarity determining regions (CDRs), optionally six antibody CDRs. In some embodiments of any of the antibodies or antigen binding fragments disclosed herein, the antigen binding protein comprises an antibody. In some embodiments of any of the antibodies or antigen binding fragments disclosed herein, the antigen binding protein is a monoclonal antibody. In some embodiments of any of the antibodies or antigen binding fragments disclosed herein, the antigen binding protein is a humanized, human, or chimeric antibody. In some embodiments of any of the antibodies or antigen binding fragments disclosed herein, the antigen binding protein is multispecific, optionally bispecific. In some embodiments of any of the antibodies or antigen binding fragments disclosed herein, the antigen binding protein binds greater than one antigen or greater than one epitope on a single antigen. In some embodiments of any of the antibodies or antigen binding fragments disclosed herein, the antigen binding protein comprises a heavy chain constant region of a class selected from IgG IgA, IgD, IgE, and IgM. In some embodiments of any of the antibodies or antigen binding fragments disclosed herein, the antigen binding protein comprises a heavy chain constant region of the class human IgG and a subclass selected from IgG1, IgG4, IgG2, and IgG3. In some embodiments of any of the antibodies or antigen binding fragments disclosed herein, the antigen binding protein comprises a modified Fc, optionally wherein the modified Fc comprises one or more mutations that extend half-life, optionally wherein the one or more mutations that extend half-life is YTE.

In another aspect, provided herein is an isolated HLA-PEPTIDE target, wherein the HLA-PEPTIDE target comprises an HLA-restricted peptide complexed with an HLA Class I molecule, wherein the HLA-restricted peptide is located in in the peptide binding groove of an α1/α2 heterodimer portion of the HLA Class I molecule, and wherein the HLA-PEPTIDE target is selected from Table A, with the proviso that the isolated HLA-PEPTIDE target is not any one of Target nos. 6364-6369, 6386-6389, 6500, 6521-6524, or 6578 and is not an HLA-PEPTIDE target found in Table B or Table C.

In some embodiments, the HLA Class I molecule is HLA subtype A*02:01 and the HLA-restricted peptide comprises the sequence LLASSILCA, the HLA Class I molecule is HLA subtype A*01:01 and the HLA-restricted peptide comprises the sequence EVDPIGHLY, the HLA Class I molecule is HLA subtype B*44:02 and the HLA-restricted peptide comprises the sequence GEMSSNSTAL, the HLA Class I molecule is HLA subtype A*02:01 and the HLA-rethe HLA Class I molecule is HLA subtype *01:01 and the HLA-restricted peptide comprises the sequence EVDPIGHVY, orstricted peptide comprises the sequence GVYDGEEHSV, the HLA Class I molecule is HLA subtype HLA-A*01:01 and the HLA-restricted peptide comprises the sequence NTDNNLAVY In some embodiments, the HLA Class I molecule is HLA subtype A*02:01 and the HLA-restricted peptide consists of or essentially consists of the sequence LLASSILCA In some embodiments, the HLA-restricted peptide is between about 5-15 amino acids in length. In some embodiments, the HLA-restricted peptide is between about 8-12 amino acids in length. In some embodiments, the association of the HLA subtype with the restricted peptide stabilizes non-covalent association of the β2-microglobin subunit of the HLA subtype with the α-subunit of the HLA subtype. In some embodiments, the stabilized association of the β2-microglobin subunit of the HLA subtype with the α-subunit of the HLA subtype is demonstrated by conditional peptide exchange. In some embodiments, the isolated HLA-PEPTIDE target further comprises an affinity tag. In some embodiments, the affinity tag is a biotin tag. In some embodiments, the isolated HLA-PEPTIDE target is complexed with a detectable label. In some embodiments, the detectable label comprises a β2-microglobin binding molecule. In some embodiments, the β2-microglobin binding molecule is a labeled antibody. In some embodiments, the labeled antibody is a fluorochrome-labeled antibody.

Also provided herein is a composition comprising an HLA-PEPTIDE target disclosed herein attached to a solid support. In some embodiments, the solid support comprises a bead, well, membrane, tube, column, plate, sepharose, magnetic bead, or chip. In some embodiments, the the HLA-PEPTIDE target comprises a first member of an affinity binding pair and the solid support comprises a second member of the affinity binding pair. In some embodiments, the first member is streptavidin and the second member is biotin.

Also provided herein is a reaction mixture comprising an isolated and purified α-subunit of an HLA subtype as described in Table A; an isolated and purified β2-microglobin subunit of the HLA subtype; an isolated and purified restricted peptide as described in Table A; and a reaction buffer.

Also provided herein is a an isolated HLA-PEPTIDE target disclosed herein; and a plurality of T-cells isolated from a human subject. In some embodiments, the the T-cells are CD8+ T-cells.

Also provided herein is an isolated polynucleotide comprising a first nucleic acid sequence encoding an HLA-restricted peptide disclosed herein, operably linked to a promoter, and a second nucleic acid sequence encoding an HLA subtype disclosed herein, wherein the second nucleic acid is operably linked to the same or different promoter as the first nucleic acid sequence, and wherein the encoded peptide and encoded HLA subtype form an HLA/peptide complex disclosed herein.

Also provided herein is a kit for expressing a stable HLA-PEPTIDE target disclosed herein, comprising a first construct comprising a first nucleic acid sequence encoding an HLA-restricted peptide disclosed herein operably linked to a promoter; and instructions for use in expressing the stable HLA-PEPTIDE complex. In some embodiments, the first construct further comprises a second nucleic acid sequence encoding an HLA subtype disclosed herein. In some embodiments, the second nucleic acid sequence is operably linked to the same or a different promoter. In some embodiments, the kit further comprises a second construct comprising a second nucleic acid sequence encoding an HLA subtype disclosed herein. In some embodiments, one or both of the first and second constructs are lentiviral vector constructs.

Also provided herein is a host cell comprising a heterologous HLA-PEPTIDE target disclosed herein. Also provided herein is a polynucleotide encoding an HLA-restricted peptide as described in Table A, e.g., a polynucleotide encoding an HLA-restricted peptide disclosed herein. In some embodiments, the does not comprise endogenous MHC. In some embodiments, the comprises an exogenous HLA. In some embodiments, the host cell is a K562 cell.

Also provided herein is a host cell as described above, and a cell culture medium comprising a restricted peptide as described in Table A. In some embodiments, the host cell is a cultured cell from a tumor cell line. In some embodiments, the tumor cell line is selected from the group consisting of HCC-1599, NCI-H510A, A375, LN229, NCI-H358, ZR-75-1, MS751, OE19, MOR, BV173, MCF-7, NCI-H82, and NCI-H146.

In some embodiments, the antigen binding protein binds to the HLA-PEPTIDE target through a contact point with the HLA Class I molecule and through a contact point with the HLA-restricted peptide of the HLA-PEPTIDE target.

In some embodiments, the ABP is for use as a medicament. In some embodiments, the ABP is for use in treatment of cancer, optionally wherein the cancer expresses or is predicted to express the HLA-PEPTIDE target. In some embodiments, the ABP is for use in treatment of cancer, wherein the cancer is selected from a solid tumor and a hematological tumor.

Also provided herein is an ABP which is a conservatively modified variant of an ABP disclosed herein. Also provided herein is an antigen binding protein (ABP) that competes for binding with the antigen binding protein disclosed herein. Also provided herein is an antigen binding protein (ABP) that binds the same HLA-PEPTIDE epitope bound by the antigen binding protein disclosed herein.

Also provided herein is an engineered cell expressing a receptor comprising the antigen binding protein disclosed herein. In some embodiments, the engineered cell is a T cell, optionally a cytotoxic T cell (CTL). In some embodiments, the antigen binding protein is expressed from a heterologous promoter.

Also provided herein is an isolated polynucleotide or set of polynucleotides encoding an antigen binding protein described herein or an antigen-binding portion thereof. Also provided herein is an isolated polynucleotide or set of polynucleotides encoding the HLA/peptide targets described herein. Also provided herein is an vector or set of vectors comprising the polynucleotide or set of polynucleotides as disclosed herein. Also provided herein is a host cell comprising the polynucleotide or set of polynucleotides disclosed herein, optionally wherein the host cell is CHO or HEK293, or optionally wherein the host cell is a T cell.

Also provided herein is a method of producing an antigen binding protein comprising expressing the antigen binding protein with the host cell as described above and isolating the expressed antigen binding protein.

Also provided herein is a pharmaceutical composition comprising the antigen binding protein disclosed herein and a pharmaceutically acceptable excipient. Also provided herein is a method of treating cancer in a subject, comprising administering to the subject an effective amount of the antigen binding protein disclosed herein or a pharmaceutical composition disclosed herein, optionally wherein the cancer is selected from a solid tumor and a hematological tumor. In some embodiments, the cancer expresses or is predicted to express the HLA-PEPTIDE target.

Also provided herein is a comprising the antigen binding protein disclosed herein or a pharmaceutical composition disclosed herein and instructions for use. Also provided herein is a composition comprising at least one HLA-PEPTIDE target disclosed herein and an adjuvant. Also provided herein is a least one HLA-PEPTIDE target disclosed herein and a pharmaceutically acceptable excipient. Also provided herein is a composition comprising an amino acid sequence comprising a polypeptide of at least one HLA-PEPTIDE target disclosed in Table A, optionally the amino acid sequence consisting essentially of or consisting of the polypeptide. Also provided herein is a virus comprising the isolated polynucleotide or set of polynucleotides disclosed herein In some embodiments, the virus is a filamentous phage. Also provided herein is a yeast cell comprising the isolated polynucleotide or set of polynucleotides disclosed herein.

Also provided herein is a method of identifying an antigen binding protein disclosed herein, comprising providing at least one HLA-PEPTIDE target listed in Table A; and binding the at least one target with the antigen binding protein, thereby identifying the antigen binding protein. In some embodiments, the antigen binding protein is present in a phage display library comprising a plurality of distinct antigen binding proteins. In some embodiments, the phage display library is substantially free of antigen binding proteins that non-specifically bind the HLA of the HLA-PEPTIDE target. In some embodiments, the antigen binding protein is present in a TCR library comprising a plurality of distinct TCRs or antigen binding fragments thereof. In some embodiments, the binding step is performed more than once, optionally at least three times. In some embodiments, the method further comprises contacting the antigen binding protein with one or more peptide-HLA complexes that are distinct from the HLA-PEPTIDE target to determine if the antigen binding protein selectively binds the HLA-PEPTIDE target, optionally wherein selectivity is determined by measuring binding affinity of the antigen binding protein to soluble target HLA-PEPTIDE complexes versus soluble HLA-PEPTIDE complexes that are distinct from target complexes, optionally wherein selectivity is determined by measuring binding affinity of the antigen binding protein to target HLA-PEPTIDE complexes expressed on the surface of one or more cells versus HLA-PEPTIDE complexes that are distinct from target complexes expressed on the surface of one or more cells.

Also provided herein is a method of identifying an antigen binding protein disclosed herein, comprising obtaining at least one HLA-PEPTIDE target listed in Table A; administering the HLA-PEPTIDE target to a subject, optionally in combination with an adjuvant; and isolating the antigen binding protein from the subject. In some embodiments, the isolating the antigen binding protein comprises screening the serum of the subject to identify the antigen binding protein. In some embodiments, the method further comprises contacting the antigen binding protein with one or more peptide-HLA complexes that are distinct from the HLA-PEPTIDE target to determine if the antigen binding protein selectively binds to the HLA-PEPTIDE target, optionally wherein selectivity is determined by measuring binding affinity of the antigen binding protein to soluble target HLA-PEPTIDE complexes versus soluble HLA-PEPTIDE complexes that are distinct from target complexes, optionally wherein selectivity is determined by measuring binding affinity of the antigen binding protein to target HLA-PEPTIDE complexes expressed on the surface of one or more cells versus HLA-PEPTIDE complexes that are distinct from target complexes expressed on the surface of one or more cells. In some embodiments, the subject is a mouse, a rabbit, or a llama. In some embodiments, the isolating the antigen binding protein comprises isolating a B cell from the subject that expresses the antigen binding protein and optionally directly cloning sequences encoding the antigen binding protein from the isolated B cell. In some embodiments, the method further comprises creating a hybridoma using the B cell. In some embodiments, the method further comprises cloning CDRs from the B cell. In some embodiments, the method further comprises immortalizing the B cell, optionally via EBV transformation. In some embodiments, the method further comprises creating a library that comprises the antigen binding protein of the B cell, optionally wherein the library is phage display or yeast display. In some embodiments, the method further comprises humanizing the antigen binding protein. Also provided herein is a method of identifying an antigen binding protein disclosed herein, comprising obtaining a cell comprising the antigen binding protein; contacting the cell with an HLA-multimer comprising at least one HLA-PEPTIDE target listed in Table A; and identifying the antigen binding protein via binding between the HLA-multimer and the antigen binding protein. Also provided herein is a method of identifying an antigen binding protein disclosed herein, comprising obtaining one or more cells comprising the antigen binding protein; activating the one or more cells with at least one HLA-PEPTIDE target listed in Table A presented on a natural or an artificial antigen presenting cell (APC); and identifying the antigen binding protein via selection of one or more cells activated by interaction with at least one HLA-PEPTIDE target listed in Table A. In some embodiments, the the cell is a T cell, optionally a CTL. In some embodiments, the method further comprises isolating the cell, optionally using flow cytometry, magnetic separation, or single cell separation. In some embodiments, the method further comprises sequencing the antigen binding protein.

Also provided herein is a method of identifying an antigen binding protein disclosed herein, comprising providing at least one HLA-PEPTIDE target listed in Table A; and identifying the antigen binding protein using the target.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows the general structure of a Human Leukocyte Antigen (HLA) Class I molecule. By User atropos235 on en.wikipedia—Own work, CC BY 2.5, https://commons.wikimedia.org/w/index.php?curid=1805424

FIG. 2 depicts exemplary construct elements for cloning TCRs into expression systems for therapy development.

FIG. 3 depicts an exemplary construct backbone sequence for cloning TCRs into expression systems for therapy development. FIG. 3 discloses SEQ ID NO: 4332.

FIG. 4 depicts an exemplary construct sequence for cloning a TCR specific for A*0201_LLASSILCA- (SEQ ID NO: 2737) into expression systems for therapy development. FIG. 4 discloses SEQ ID NO: 4333.

FIG. 5 depicts an exemplary exemplary construct sequence for cloning a TCR specific for A*0101_EVDPIGHLY (SEQ ID NO: 1) into expression systems for therapy development. FIG. 5 discloses SEQ ID NO: 4334.

FIG. 6 shows spectra data for peptide EVDPIGHLY (SEQ ID NO: 1). The figure contains the peptide fragmentation information as well as information related to the patient sample, including HLA types.

FIG. 7 shows spectra data for peptide GVHGGILNK (SEQ ID NO: 1424). The figure contains the peptide fragmentation information as well as information related to the patient sample, including HLA types.

FIG. 7A shows spectra data for peptide GVYDGEEHSV

FIG. 7B shows spectra data for peptide NTDNNLAVY

FIGS. 7C-7K show spectra data for additional peptides disclosed in Table A.

FIG. 8 shows the design of target screen 1 for the G2 target HLA-A*01:01_NTDNNLAVY (SEQ ID NO: 23).

FIG. 9A shows the target and minipool negative control design for the G2 target. FIG. 9A discloses SEQ ID NOS 23 and 4335-4337, respectively, in order of appearance.

FIG. 9B shows stability ELISA results for the G2 counterscreen “minipool” and G2 targets. FIG. 9B discloses SEQ ID NOS 23, 4335-4337 and 4363, respectively, in order of appearance.

FIG. 10 shows stability ELISA results for the additional G2 “complete” pool counterscreen peptides. FIG. 10 discloses SEQ ID NOS 4338-4352, respectively, in order of appearance.

FIG. 11 shows shows the design of target screen 2 for the G7 target HLA-A*02:01 LLASSILCA (SEQ ID NO: 2737).

FIG. 12 shows stability ELISA results for the additional G7 “complete pool” counterscreen peptides. FIG. 12 discloses SEQ ID NOS 4341-4343, 4350-4358 and 4335-4337, respectively, in order of appearance.

FIG. 13A shows the target and minipool negative control design for the G7 target. FIG. 13A discloses SEQ ID NOS 2737 and 4338-4340, respectively, in order of appearance.

FIG. 13B shows stability ELISA results for the G7 counterscreen “minipool” and G7 targets. FIG. 13B discloses SEQ ID NOS 2737, 4338-4340 and 4344, respectively, in order of appearance.

FIGS. 14A and 14B show phage panning results for the G2 and G7 targets, respectively.

FIGS. 15A and 15B show biolayer interferometry (BLI) results for G2 target Fab clone G-2P1H11 and G7 target G7R4-B5-P2E9, respectively.

FIG. 16 shows a map of the amino acid substitutions for the positional scanning experiment described herein. FIG. 16 discloses SEQ ID NOS 23 and 2737, respectively, in order of appearance.

FIG. 17A shows a stability heat map for the G2 positional variant-HLAs. FIG. 17A discloses SEQ ID NO: 23.

FIG. 17B shows an affinity heat map for Fab clone G2-P1H11. FIG. 17B discloses SEQ ID NO: 23.

FIG. 18A shows a stability heat map for the G7 positional variants. FIG. 18A discloses SEQ ID NO: 2737.

FIG. 18B shows an affinity heat map for Fab clone G7R4-B5-P2E9. FIG. 18B discloses SEQ ID NO: 2737.

FIG. 19 shows cell binding results for Fab clones G2-P1H11 and G7R4-B5-P2E9 to HLA-transduced K562 cells pulsed with target or negative control peptides.

FIG. 20 shows cell binding results for Fab clones G2-P1H11 and G7R4-B5-P2E9 to HLA-transduced K562 cells pulsed with target or negative control peptides.

FIG. 21 shows an example of hydrogen-deuterium exchange (HDX) data plotted on a crystal structure PDB 5bs0.

FIG. 22 shows an exemplary HDX heatmap for scFv clone G2-P1G07 visualized in its entirety using a consolidated perturbation view. FIG. 22 discloses SEQ ID NO: 4359.

FIG. 23 shows HDX heat maps across the HLA α1 and α2 helices for the tested G2 scFv and Fab clones. FIG. 23 discloses SEQ ID NOS 4360-4361, respectively, in order of appearance.

FIG. 24 shows an HDX heat map across the restricted peptide NTDNNLAVY (SEQ ID NO: 23) for the tested G2 scFv and Fab clones.

FIG. 25 depicts an experimental workflow by which TCRs which specifically bind HLA-PEPTIDE targets were isolated.

FIG. 26 shows a flow cytometry sorting procedure for sorting MHC-target-specific CD8+ T cells.

FIG. 27 shows flow cytometry results for exemplary HLA-PEPTIDE targets B*44:02_GEMSSNSTAL (SEQ ID NO: 2721) and A*01:01_EVDPIGHLY (SEQ ID NO: 1).

FIG. 28 shows flow cytometry results for the HLA-PETPIDE target A*03:01_GVHGGILNK (SEQ ID NO: 1424). FIG. 28 also discloses “EVDPIGHVY” as SEQ ID NO: 6.

FIG. 29A shows total number of isolated CD8+ T cells per HLA-PEPTIDE target summed across all donors tested. FIG. 29A discloses SEQ ID NOS 23, 302, 2737, 96, 1424, 2721, 6 and 1, respectively, in order of appearance.

FIG. 29B shows frequency of isolated CD8+ T cells per HLA-PEPTIDE target summed across all donors tested. FIG. 29B discloses SEQ ID NOS 1, 2737, 302, 1424, 6, 2721, 96 and 23, respectively, in order of appearance.

FIG. 30A depicts the number of unique TCR clonotypes per HLA-PEPTIDE target for each tested donor. FIG. 30A discloses SEQ ID NOS 23, 2737, 96, 1424, 2721, 6 and 1, respectively, in order of appearance.

FIG. 30B depicts the total number of unique clonotypes per HLA-PEPTIDE target, summed across all donors tested. FIG. 30B discloses SEQ ID NOS 23, 2737, 96, 1424, 2721, 6 and 1, respectively, in order of appearance.

FIG. 31 shows examples of Jurkat cells expressing A*0201_LLASSILCA- (SEQ ID NO: 2737), A*0201_GVYDGEEHSV- (SEQ ID NO: 96), B*4402_GEMSSNSTAL-(SEQ ID NO: 2721), and A*0101_EVDPIGHLY (SEQ ID NO: 1)-specific TCRs binding to their respective HLA-PEPTIDE targets but not to the control peptide tetramer.

FIG. 32 shows the gating strategy and flow data demonstrating that human CD8+ cells transduced with TCRs identified herein bind to their specific HLA-PEPTIDE target. FIG. 32 discloses SEQ ID NOS 2737 and 2737, respectively, in order of appearance.

FIG. 33 shows an exemplary lentiviral vector useful for transducing recipient cells with a TCR disclosed herein.

### DETAILED DESCRIPTION

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise. For example, a multispecific ABP “comprising a diabody” includes a multispecific ABP “consisting of a diabody” and a multispecific ABP “consisting essentially of a diabody.”

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s) ±one standard deviation of that value(s).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, Pa. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antigen binding protein” or “ABP” is used herein in its broadest sense and includes certain types of molecules comprising one or more antigen-binding domains that specifically bind to an antigen or epitope.

In some embodiments, the ABP comprises an antibody. In some embodiments, the ABP consists of an antibody. In some embodiments, the ABP consists essentially of an antibody. An ABP specifically includes intact antibodies (e.g., intact immunoglobulins), antibody fragments, ABP fragments, and multi-specific antibodies. In some embodiments, the ABP comprises an alternative scaffold. In some embodiments, the ABP consists of an alternative scaffold. In some embodiments, the ABP consists essentially of an alternative scaffold. In some embodiments, the ABP comprises an antibody fragment. In some embodiments, the ABP consists of an antibody fragment. In some embodiments, the ABP consists essentially of an antibody fragment. In some embodiments, the ABP comprises a TCR or antigen binding portion thereof. In some embodiments, the ABP consists of a TCR or antigen binding portion thereof. In some embodiments, the ABP consists essentially of a TCR or antigen binding portion thereof. In some embodiments, a CAR comprises an ABP. An “HLA-PEPTIDE ABP,” “anti-HLA-PEPTIDE ABP,” or “HLA-PEPTIDE-specific ABP” is an ABP, as provided herein, which specifically binds to the antigen HLA-PEPTIDE. An ABP includes proteins comprising one or more antigen-binding domains that specifically bind to an antigen or epitope via a variable region, such as a variable region derived from a B cell (e.g., antibody) or T cell (e.g., TCR).

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

As used herein, “variable region” refers to a variable nucleotide sequence that arises from a recombination event, for example, it can include a V, J, and/or D region of an immunoglobulin or T cell receptor (TCR) sequence from a B cell or T cell, such as an activated T cell or an activated B cell.

The term “antigen-binding domain” means the portion of an ABP that is capable of specifically binding to an antigen or epitope. One example of an antigen-binding domain is an antigen-binding domain formed by an antibody V_{H}-V_{L} dimer of an ABP. Another example of an antigen-binding domain is an antigen-binding domain formed by diversification of certain loops from the tenth fibronectin type III domain of an Adnectin. An antigen-binding domain can include antibody CDRs 1, 2, and 3 from a heavy chain in that order; and antibody CDRs 1, 2, and 3 from a light chain in that order. An antigen-binding domain can include TCR CDRs, e.g., αCDR1, αCDR2, αCDR3, βCDR1, βCDR2, and βCDR3. TCR CDRs are described herein.

The antibody V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three antibody CDRs and four FRs, arranged in the following order (from N-terminus to C-terminus): FR1-CDR1-FR2-CDR2-FR3-CDR3-FR4. The antibody CDRs are involved in antigen binding, and influence antigen specificity and binding affinity of the ABP. See Kabat et al., Sequences of Proteins of Immunological Interest 5th ed. (1991) Public Health Service, National Institutes of Health, Bethesda, Md., incorporated by reference in its entirety.

The light chain from any vertebrate species can be assigned to one of two types, called kappa (κ) and lambda (λ), based on the sequence of its constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of an antibody CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme); each of which is incorporated by reference in its entirety.

Table 14 provides the positions of antibody CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

Antibody CDRs may be assigned, for example, using ABP numbering software, such as Abnum, available at www.bioinf.org.uk/abs/abnum/, and described in Abhinandan and Martin, Immunology, 2008, 45:3832-3839, incorporated by reference in its entirety.

TABLE 14
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an ABP heavy chain constant region (e.g., as reported in Kabat et al., supra). Unless stated otherwise, the EU numbering scheme is used to refer to residues in ABP heavy chain constant regions described herein.

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The amino acid sequence boundaries of a TCR CDR can be determined by one of skill in the art using any of a number of known numbering schemes, including but not limited to the IMGT unique numbering, as described by LeFranc, M.-P, Immunol Today. 1997 November; 18(11):509; Lefranc, M.-P., “IMGT Locus on Focus: A new section of Experimental and Clinical Immunogenetics”, Exp. Clin. Immunogenet., 15, 1-7 (1998); Lefranc and Lefranc, The T Cell Receptor FactsBook; and M.-P. Lefranc/Developmental and Comparative Immunology 27 (2003) 55-77, all of which are incorporated by reference.

An “ABP fragment” comprises a portion of an intact ABP, such as the antigen-binding or variable region of an intact ABP. ABP fragments include, for example, Fv fragments, Fab fragments, F(ab′)₂ fragments, Fab′ fragments, scFv (sFv) fragments, and scFv-Fc fragments. ABP fragments include antibody fragments. Antibody fragments can include Fv fragments, Fab fragments, F(ab′)₂ fragments, Fab′ fragments, scFv (sFv) fragments, scFv-Fc fragments, and TCR fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length ABP.

“F(ab′)₂” fragments contain two Fab′ fragments joined, near the hinge region, by disulfide bonds. F(ab′)₂ fragments may be generated, for example, by recombinant methods or by pepsin digestion of an intact ABP. The F(ab′) fragments can be dissociated, for example, by treatment with ß-mercaptoethanol.

“Single-chain Fv” or “sFv” or “scFv” fragments comprise a V_{H} domain and a V_{L} domain in a single polypeptide chain. The V_{H} and V_{L} are generally linked by a peptide linker. See Plückthun A. (1994). Any suitable linker may be used. In some embodiments, the linker is a (GGGGS)ₙ. In some embodiments, n=1, 2, 3, 4, 5, or 6. See ABPs from Escherichia coli. In Rosenberg M. & Moore G. P. (Eds.), The Pharmacology of Monoclonal ABPs vol. 113 (pp. 269-315). Springer-Verlag, New York, incorporated by reference in its entirety.

“scFv-Fc” fragments comprise an scFv attached to an Fc domain. For example, an Fc domain may be attached to the C-terminal of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG4 Fc domain.

The term “single domain antibody” refers to a molecule in which one variable domain of an ABP specifically binds to an antigen without the presence of the other variable domain. Single domain ABPs, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain ABPs are also known as sdAbs or nanobodies.

The term “Fc region” or “Fc” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125:S41-52, incorporated by reference in its entirety. The Fc region may be a naturally occurring Fc region, or an Fc region modified as described in the art or elsewhere in this disclosure.

The term “alternative scaffold” refers to a molecule in which one or more regions may be diversified to produce one or more antigen-binding domains that specifically bind to an antigen or epitope. In some embodiments, the antigen-binding domain binds the antigen or epitope with specificity and affinity similar to that of an ABP. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody®), ankyrin repeats (e.g., DARPins), gamma-B-crystallin/ubiquitin (e.g., Affilins), CTLD₃ (e.g., Tetranectins), Fynomers, and (LDLR-A module) (e.g., Avimers). Additional information on alternative scaffolds is provided in Binz et al., Nat. Biotechnol., 2005 23:1257-1268; Skerra, Current Opin. in Biotech., 2007 18:295-304; and Silacci et al., J Biol. Chem., 2014, 289:14392-14398; each of which is incorporated by reference in its entirety. An alternative scaffold is one type of ABP.

A “multispecific ABP” is an ABP that comprises two or more different antigen-binding domains that collectively specifically bind two or more different epitopes. The two or more different epitopes may be epitopes on the same antigen (e.g., a single HLA-PEPTIDE molecule expressed by a cell) or on different antigens (e.g., different HLA-PEPTIDE molecules expressed by the same cell, or a HLA-PEPTIDE molecule and a non-HLA-PEPTIDE molecule). In some aspects, a multi-specific ABP binds two different epitopes (i.e., a “bispecific ABP”). In some aspects, a multi-specific ABP binds three different epitopes (i.e., a “trispecific ABP”).

A “monospecific ABP” is an ABP that comprises one or more binding sites that specifically bind to a single epitope. An example of a monospecific ABP is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

“Affinity” refers to the strength of the sum total of non-covalent interactions between a single binding site of a molecule (e.g., an ABP) and its binding partner (e.g., an antigen or epitope). Unless indicated otherwise, as used herein, “affinity” refers to intrinsic binding affinity, which reflects a 1:1 interaction between members of a binding pair (e.g., ABP and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an ABP to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the ABP to the target molecule is competitively inhibited by the control molecule. In some aspects, the affinity of a HLA-PEPTIDE ABP for a non-target molecule is less than about 50% of the affinity for HLA-PEPTIDE. In some aspects, the affinity of a HLA-PEPTIDE ABP for a non-target molecule is less than about 40% of the affinity for HLA-PEPTIDE. In some aspects, the affinity of a HLA-PEPTIDE ABP for a non-target molecule is less than about 30% of the affinity for HLA-PEPTIDE. In some aspects, the affinity of a HLA-PEPTIDE ABP for a non-target molecule is less than about 20% of the affinity for HLA-PEPTIDE. In some aspects, the affinity of a HLA-PEPTIDE ABP for a non-target molecule is less than about 10% of the affinity for HLA-PEPTIDE. In some aspects, the affinity of a HLA-PEPTIDE ABP for a non-target molecule is less than about 1% of the affinity for HLA-PEPTIDE. In some aspects, the affinity of a HLA-PEPTIDE ABP for a non-target molecule is less than about 0.1% of the affinity for HLA-PEPTIDE.

The term “k_{d}” (sec⁻¹), as used herein, refers to the dissociation rate constant of a particular ABP—antigen interaction. This value is also referred to as the k_{off} value.

The term “kₐ” (M⁻¹×sec⁻¹), as used herein, refers to the association rate constant of a particular ABP-antigen interaction. This value is also referred to as the kₒₙ value.

The term “K_{D}” (M), as used herein, refers to the dissociation equilibrium constant of a particular ABP-antigen interaction. K_{D}=k_{d}/kₐ. In some embodiments, the affinity of an ABP is described in terms of the K_{D} for an interaction between such ABP and its antigen. For clarity, as known in the art, a smaller K_{D} value indicates a higher affinity interaction, while a larger K_{D} value indicates a lower affinity interaction.

The term “K_{A}” (M⁻¹), as used herein, refers to the association equilibrium constant of a particular ABP-antigen interaction. K_{A}=kₐ/k_{d}.

An “immunoconjugate” is an ABP conjugated to one or more heterologous molecule(s), such as a therapeutic (cytokine, for example) or diagnostic agent.

“Fc effector functions” refer to those biological activities mediated by the Fc region of an ABP having an Fc region, which activities may vary depending on isotype. Examples of ABP effector functions include C1q binding to activate complement dependent cytotoxicity (CDC), Fc receptor binding to activate ABP-dependent cellular cytotoxicity (ADCC), and ABP dependent cellular phagocytosis (ADCP).

When used herein in the context of two or more ABPs, the term “competes with” or “cross-competes with” indicates that the two or more ABPs compete for binding to an antigen (e.g., HLA-PEPTIDE). In one exemplary assay, HLA-PEPTIDE is coated on a surface and contacted with a first HLA-PEPTIDE ABP, after which a second HLA-PEPTIDE ABP is added. In another exemplary assay, a first HLA-PEPTIDE ABP is coated on a surface and contacted with HLA-PEPTIDE, and then a second HLA-PEPTIDE ABP is added. If the presence of the first HLA-PEPTIDE ABP reduces binding of the second HLA-PEPTIDE ABP, in either assay, then the ABPs compete with each other. The term “competes with” also includes combinations of ABPs where one ABP reduces binding of another ABP, but where no competition is observed when the ABPs are added in the reverse order. However, in some embodiments, the first and second ABPs inhibit binding of each other, regardless of the order in which they are added. In some embodiments, one ABP reduces binding of another ABP to its antigen by at least 25%, at least 50%, at least 60%, at least 70%, at least 80%, at least 85%, at least 90%, or at least 95%. A skilled artisan can select the concentrations of the ABPs used in the competition assays based on the affinities of the ABPs for HLA-PEPTIDE and the valency of the ABPs. The assays described in this definition are illustrative, and a skilled artisan can utilize any suitable assay to determine if ABPs compete with each other. Suitable assays are described, for example, in Cox et al., “Immunoassay Methods,” in Assay Guidance Manual [Internet], Updated Dec. 24, 2014 (www.ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

The term “epitope” means a portion of an antigen that specifically binds to an ABP. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an ABP binds can be determined using known techniques for epitope determination such as, for example, testing for ABP binding to HLA-PEPTIDE variants with different point-mutations, or to chimeric HLA-PEPTIDE variants.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. By way of example, the groups of amino acids provided in Tables 15-17 are, in some embodiments, considered conservative substitutions for one another.

TABLE 15
Selected groups of amino acids that are considered conservative
substitutions for one another, in certain embodiments.

| | |
|---|---|
| Acidic Residues | D and E |
| Basic Residues | K, R and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 16
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L and M |
| Group 6 | F, Y, and W |

TABLE 17
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, N.Y. An ABP generated by making one or more conservative substitutions of amino acid residues in a parent ABP is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “therapeutically effective amount” or “effective amount” refers to an amount of an ABP or pharmaceutical composition provided herein that, when administered to a subject, is effective to treat a disease or disorder.

As used herein, the term “subject” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an ABP provided herein. In some aspects, the disease or condition is a cancer. In some aspects, the disease or condition is a viral infection.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is a cancer. In some aspects, the tumor is a solid tumor. In some aspects, the tumor is a hematologic malignancy.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

The terms “nucleic acids” and “polynucleotides” may be used interchangeably herein to refer to polymeric form of nucleotides of any length, either deoxyribonucleotides or ribonucleotides, or analogs thereof. Polynucleotides can include, but are not limited to coding or non-coding regions of a gene or gene fragment, loci (locus) defined from linkage analysis, exons, introns, messenger RNA (mRNA), cDNA, recombinant polynucleotides, branched polynucleotides, plasmids, vectors, isolated DNA, isolated RNA, nucleic acid probes, and primers. A polynucleotide may comprise modified nucleotides, such as methylated nucleotides and nucleotide analogs. Exemplary modified nucleotides include, e.g., 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthioN6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

Isolated HLA-Peptide Targets

The major histocompatibility complex (MHC) is a complex of antigens encoded by a group of linked loci, which are collectively termed H-2 in the mouse and HLA in humans. The two principal classes of the MHC antigens, class I and class II, each comprise a set of cell surface glycoproteins which play a role in determining tissue type and transplant compatibility. In transplantation reactions, cytotoxic T-cells (CTLs) respond mainly against class I glycoproteins, while helper T-cells respond mainly against class II glycoproteins.

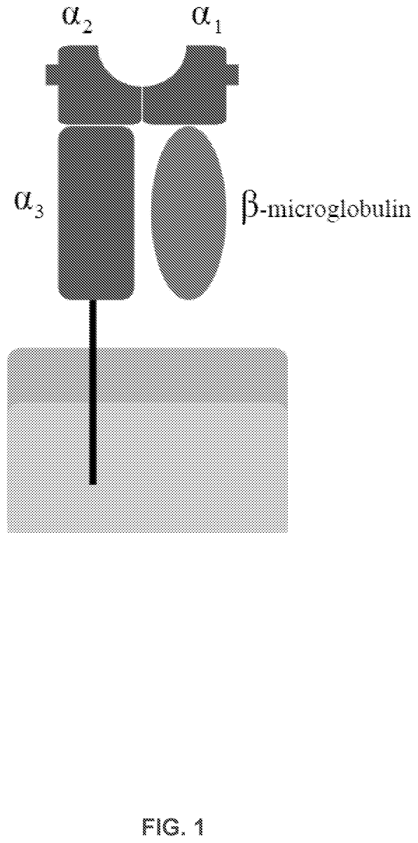

Human major histocompatibility complex (MHC) class I molecules, referred to interchangeably herein as HLA Class I molecules, are expressed on the surface of nearly all cells. These molecules function in presenting peptides which are mainly derived from endogenously synthesized proteins to CD8+ T cells via an interaction with the alpha-beta T-cell receptor. The class I MHC molecule comprises a heterodimer composed of a 46-kDa a chain which is non-covalently associated with the 12-kDa light chain beta-2 microglobulin. The α chain generally comprises α1 and α2 domains which form a groove for presenting an HLA-restricted peptide, and an α3 plasma membrane-spanning domain which interacts with the CD8 co-receptor of T-cells. FIG. 1 (prior art) depicts the general structure of a Class I HLA molecule.

Class I MHC-restricted peptides (also referred to interchangeably herein as HLA-restricted antigens, HLA-restricted peptides, MHC-restricted antigens, restricted peptides, or peptides) generally bind to the heavy chain alpha1-alpha2 groove via about two or three anchor residues that interact with corresponding binding pockets in the MHC molecule. The beta-2 microglobulin chain plays an important role in MHC class I intracellular transport, peptide binding, and conformational stability. For most class I molecules, the formation of a heterotrimeric complex of the MHC class I heavy chain, peptide (self, non-self, and/or antigenic) and beta-2 microglobulin leads to protein maturation and export to the cell-surface.

Binding of a given HLA subtype to an HLA-restricted peptide forms a complex with a unique and novel surface that can be specifically recognized by an ABP such as, e.g., a TCR on a T cell or an antibody or antigen-binding fragment thereof. HLA complexed with an HLA-restricted peptide is referred to herein as an HLA-PEPTIDE or HLA-PEPTIDE target. In some cases the restricted peptide is located in the α1/α2 groove of the HLA molecule. In some cases the restricted peptide is bound to the α1/α2 groove of the HLA molecule via about two or three anchor residues that interact with corresponding binding pockets in the HLA molecule.

Accordingly, provided herein are antigens comprising HLA-PEPTIDE targets. The HLA-PEPTIDE targets may comprise a specific HLA-restricted peptide having a defined amino acid sequence complexed with a specific HLA subtype.

HLA-PEPTIDE targets identified herein may be useful for cancer immunotherapy. In some embodiments, the HLA-PEPTIDE targets identified herein are presented on the surface of a tumor cell. The HLA-PEPTIDE targets identified herein may be expressed by tumor cells in a human subject. The HLA-PEPTIDE targets identified herein may be expressed by tumor cells in a population of human subjects. For example, the HLA-PEPTIDE targets identified herein may be shared antigens which are commonly expressed in a population of human subjects with cancer.

The HLA-PEPTIDE targets identified herein may have a prevalence with an individual tumor type The prevalence with an individual tumor type may be about 0.1%, 0.2%, 0.3%, 0.4%, 0.5%, 0.6%, 0.7%, 0.8%, 0.9%, 1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. The prevalence with an individual tumor type may be about 0.1%-100%, 0.2-50%, 0.5-25%, or 1-10%.

Preferably, HLA-PEPTIDE targets are not generally expressed in most normal tissues. For example, the HLA-PEPTIDE targets may in some cases not be expressed in tissues in the Genotype-Tissue Expression (GTEx) Project, or may in some cases be expressed only in immune privileged or non-essential tissues. Exemplary immune privileged or non-essential tissues include testis, minor salivary glands, the endocervix, and the thyroid. In some cases, an HLA-PEPTIDE target may be deemed to not be expressed on essential tissues or non-immune privileged tissues if the median expression of a gene from which the restricted peptide is derived is less than 0.5 RPKM (Reads Per Kilobase of transcript per Million napped reads) across GTEx samples, if the gene is not expressed with greater than 10 RPKM across GTEX samples, if the gene was expressed at >=5 RPKM in no more two samples across all essential tissue samples, or any combination thereof.

Exemplary HLA Class I Subtypes of the HLA-PEPTIDE Targets

In humans, there are many MHC haplotypes (referred to interchangeably herein as MHC subtypes, HLA subtypes, MHC types, and HLA types). Exemplary HLA subtypes include, by way of example only, HLA-A2, HLA-A1, HLA-A3, HLA-A11, HLA-A23, HLA-A24, HLA-A25, HLA-A26, HLA-A28, HLA-A29, HLA-A30, HLA-A31, HLA-A32, HLA-A33, HLA-A34, HLA-68, HLA-B7, HLA-B8, HLA-B40, HLA-B44, HLA-B13, HLA-B15, HLA-B-18, HLA-B27, HLA-B35, HLA-B37, HLA-B38, HLA-B39, HLA-B45, HLA-B46, HLA-B49, HLA-B51, HLA-B54, HLA-B55, HLA-B56, HLA-B57, HLA-B58, HLA-C*01, HLA-C*02, HLA-C*03, HLA-C*04, HLA-C*05, HLA-C*06, HLA-C*07, HLA-C*12, HLA-C*14, HLA-C*16, HLA-Cw8, and all 4 digit and 6 digit subtypes thereof. As is known to those skilled in the art there are allelic variants of the above HLA types, all of which are encompassed by the present invention. A full list of HLA Class Alleles can be found on http://hla.alleles.org/alleles/. For example, a full list of HLA Class I Alleles can be found on http://hla.alleles.org/alleles/class1.html.

HLA-Restricted Peptides

The HLA-restricted peptides (referred to interchangeably herein) as “restricted peptides” can be peptide fragments of tumor-specific genes, e.g., cancer-specific genes. Preferably, the cancer-specific genes are expressed in cancer samples. Genes which are aberrantly expressed in cancer samples can be identified through a database. Exemplary databases include, by way of example only, The Cancer Genome Atlas (TCGA) Research Network: http://cancergenome.nih.gov/; the International Cancer Genome Consortium: https://dcc.icgc.org/. In some embodiments, the cancer-specific gene has an observed expression of at least 10 RPKM in at least 5 samples from the TCGA database. The cancer-specific gene may have an observable bimodal distribution

The cancer-specific gene may have an observed expression of greater than 10, 20, 30, 40, 50, 60, 70, 80, 90, or 100 TPM in at least one TCGA tumor tissue. In preferred embodiments, the cancer-specific gene has an observed expression of greater than 100 TPM in at least one TCGA tumor tissue. In some cases, the cancer specific gene has an observed bimodal distribution of expression across TCGA samples. Without wishing to be bound by theory, such bimodal expression pattern is consistent with a biological model in which there is minimal expression at baseline in all tumor samples and higher expression in a subset of tumors experiencing epigenetic dysregulation.

Preferably, the cancer-specific gene is not generally expressed in most normal tissues. For example, the cancer-specific gene may in some cases not be expressed in tissues in the Genotype-Tissue Expression (GTEx) Project, or may in some cases be expressed in immune privileged or non-essential tissues. Exemplary immune privileged or non-essential tissues include testis, minor salivary glands, the endocervix, and thyroid. In some cases, an cancer-specific gene may be deemed to not be expressed an essential tissues or non-immune privileged tissue if the median expression of the cancer-specific gene is less than 0.5 RPKM (Reads Per Kilobase of transcript per Million napped reads) across GTEx samples, if the gene is not expressed with greater than 10 RPKM across GTEX samples, if the gene was expressed at >=5 RPKM in no more two samples across all essential tissue samples, or any combination thereof.

In some embodiments, the cancer-specific gene meets the following criteria by assessment of the GTEx: (1) median GTEx expression in brain, heart, or lung is less than 0.1 transcripts per million (TPM), with no one sample exceeding 5 TPM, (2) median GTEx expression in other essential organs (excluding testis, thyroid, minor salivary gland) is less than 2 TPM with no one sample exceeding 10 TPM.

In some embodiments, the cancer-specific gene is not likely expressed in immune cells generally, e.g., is not an interferon family gene, is not an eye-related gene, not an olfactory or taste receptor gene, and is not a gene related to the circadian cycle (e.g., not a CLOCK, PERIOD, CRY gene)

The restricted peptide preferably may be presented on the surface of a tumor.

The restricted peptides may have a size of about 5, about 6, about 7, about 8, about 9, about 10, about 11, about 12, about 13, about 14, or about 15 amino molecule residues, and any range derivable therein. In particular embodiments, the restricted peptide has a size of about 8, about 9, about 10, about 11, or about 12 amino molecule residues. The restricted peptide may be about 5-15 amino acids in length, preferably may be about 7-12 amino acids in length, or more preferably may be about 8-11 amino acids in length.

Exemplary HLA-PEPTIDE Targets

Exemplary HLA-PEPTIDE targets are shown in Table A. In each row of Table A the HLA allele and corresponding HLA-restricted peptide sequence of each complex is shown. The peptide sequence can consist of the respective sequence shown in each row of Table A. Alternatively the peptide sequence can comprise the respective sequence shown in each row of Table A. Alternatively the peptide sequence can consist essentially of the respective sequence shown in each row of Table A.

In some embodiments, the HLA-PEPTIDE target is a target as shown in Table A.

In some embodiments, the HLA-PEPTIDE target is a target shown in Table A, with the proviso that the isolated HLA-PEPTIDE target is not any one of Target nos. 6364-6369, 6386-6389, 6500, 6521-6524, or 6578 and is not an HLA-PEPTIDE target found in Table B or Table C.

In some embodiments, the HLA-restricted peptide is not from a gene selected from WT1 or MART1.

HLA Class I molecules which do not associate with a restricted peptide ligand are generally unstable. Accordingly, the association of the restricted peptide with the α1/α2 groove of the HLA molecule may stabilize the non-covalent association of the β2-microglobin subunit of the HLA subtype with the α-subunit of the HLA subtype.

Stability of the non-covalent association of the β2-microglobin subunit of the HLA subtype with the α-subunit of the HLA subtype can be determined using any suitable means. For example, such stability may be assessed by dissolving insoluble aggregates of HLA molecules in high concentrations of urea (e.g., about 8M urea), and determining the ability of the HLA molecule to refold in the presence of the restricted peptide during urea removal, e.g., urea removal by dialysis. Such refolding approaches are described in, e.g., Proc. Natl. Acad. Sci. USA Vol. 89, pp. 3429-3433, April 1992, hereby incorporated by reference.

For other example, such stability may be assessed using conditional HLA Class I ligands. Conditional HLA Class I ligands are generally designed as short restricted peptides which stabilize the association of the β2 and α subunits of the HLA Class I molecule by binding to the α1/α2 groove of the HLA molecule, and which contain one or more amino acid modifications allowing cleavage of the restricted peptide upon exposure to a conditional stimulus. Upon cleavage of the conditional ligand, the β2 and α-subunits of the HLA molecule dissociate, unless such conditional ligand is exchanged for a restricted peptide which binds to the α1/α2 groove and stabilizes the HLA molecule. Conditional ligands can be designed by introducing amino acid modifications in either known HLA peptide ligands or in predicted high-affinity HLA peptide ligands. For HLA alleles for which structural information is available, water-accessibility of side chains may also be used to select positions for introduction of the amino acid modifications. Use of conditional HLA ligands may be advantageous by allowing the batch preparation of stable HLA-peptide complexes which may be used to interrogate test restricted peptides in a high throughput manner. Conditional HLA Class I ligands, and methods of production, are described in, e.g., Proc Natl Acad Sci USA. 2008 Mar. 11; 105(10): 3831-3836; Proc Natl Acad Sci USA. 2008 Mar. 11; 105(10): 3825-3830; J Exp Med. 2018 May 7; 215(5): 1493-1504; Choo, J. A. L. et al. Bioorthogonal cleavage and exchange of major histocompatibility complex ligands by employing azobenzene-containing peptides. Angew Chem Int Ed Engl 53, 13390-13394 (2014); Amore, A. et al. Development of a Hypersensitive Periodate-Cleavable Amino Acid that is Methionine- and Disulfide-Compatible and its Application in MHC Exchange Reagents for T Cell Characterisation. ChemBioChem 14, 123-131 (2012); Rodenko, B. et al. Class I Major Histocompatibility Complexes Loaded by a Periodate Trigger. J Am Chem Soc 131, 12305-12313 (2009); and Chang, C. X. L. et al. Conditional ligands for Asian HLA variants facilitate the definition of CD8+ T-cell responses in acute and chronic viral diseases. Eur J Immunol 43, 1109-1120 (2013). These references are incorporated by reference in their entirety.

Accordingly, in some embodiments, the ability of an HLA-restricted peptide described herein, e.g., described in Table A, to stabilize the association of the β2- and α-subunits of the HLA molecule, is assessed by performing a conditional ligand mediated-exchange reaction and assay for HLA stability. HLA stability can be assayed using any suitable method, including, e.g., mass spectrometry analysis, immunoassays (e.g., ELISA), size exclusion chromatography, and HLA multimer staining followed by flow cytometry assessment of T cells.

Other exemplary methods for assessing stability of the non-covalent association of the β2-microglobin subunit of the HLA subtype with the α-subunit of the HLA subtype include peptide exchange using dipeptides. Peptide exchange using dipeptides has been described in, e.g., Proc Natl Acad Sci USA. 2013 Sep. 17, 110(38): 15383-8; Proc Natl Acad Sci USA. 2015 Jan. 6, 112(1):202-7, which is hereby incorporated by reference.

Provided herein are useful antigens comprising an HLA-PEPTIDE target. The HLA-PEPTIDE targets may comprise a specific HLA-restricted peptide having a defined amino acid sequence complexed with a specific HLA subtype allele.

The HLA-PEPTIDE target may be isolated and/or in substantially pure form. For example, the HLA-PEPTIDE targets may be isolated from their natural environment, or may be produced by means of a technical process. In some cases, the HLA-PEPTIDE target is provided in a form which is substantially free of other peptides or proteins.

THE HLA-PEPTIDE targets may be presented in soluble form, and optionally may be a recombinant HLA-PEPTIDE target complex. The skilled artisan may use any suitable method for producing and purifying recombinant HLA-PEPTIDE targets. Suitable methods include, e.g., use of E. coli expression systems, insect cells, and the like. Other methods include synthetic production, e.g., using cell free systems. An exemplary suitable cell free system is described in WO2017089756, which is hereby incorporated by reference in its entirety.

Also provided herein are compositions comprising an HLA-PEPTIDE target.

In some cases, the composition comprises an HLA-PEPTIDE target attached to a solid support. Exemplary solid supports include, but are not limited to, beads, wells, membranes, tubes, columns, plates, sepharose, magnetic beads, and chips. Exemplary solid supports are described in, e.g., Catalysts 2018, 8, 92; doi:10.3390/catal8020092, which is hereby incorporated by reference in its entirety.

The HLA-PEPTIDE target may be attached to the solid support by any suitable methods known in the art. In some cases, the HLA-PEPTIDE target is covalently attached to the solid support.

In some cases, the HLA-PEPTIDE target is attached to the solid support by way of an affinity binding pair. Affinity binding pairs generally involved specific interactions between two molecules. A ligand having an affinity for its binding partner molecule can be covalently attached to the solid support, and thus used as bait for immobilizing Common affinity binding pairs include, e.g., streptavidin and biotin, avidin and biotin; polyhistidine tags with metal ions such as copper, nickel, zinc, and cobalt; and the like.

The HLA-PEPTIDE target may comprise a detectable label.

Pharmaceutical compositions comprising HLA-PEPTIDE targets.

The composition comprising an HLA-PEPTIDE target may be a pharmaceutical composition. Such a composition may comprise multiple HLA-PEPTIDE targets. Exemplary pharmaceutical compositions are described herein. The composition may be capable of eliciting an immune response. The composition may comprise an adjuvant. Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

HLA-Peptide ABPs

Also provided herein are ABPs that specifically bind to HLA-PEPTIDE target as disclosed herein.

The HLA-PEPTIDE target may be expressed on the surface of any suitable target cell including a tumor cell.

The ABP can specifically bind to a human leukocyte antigen (HLA)-PEPTIDE target, wherein the HLA-PEPTIDE target comprises an HLA-restricted peptide complexed with an HLA Class I molecule, wherein the HLA-restricted peptide is located in the peptide binding groove of an α1/α2 heterodimer portion of the HLA Class I molecule.

In some aspects, the ABP does not bind HLA class I in the absence of HLA-restricted peptide. In some aspects, the ABP does not bind HLA-restricted peptide in the absence of human MHC class I. In some aspects, the ABP binds tumor cells presenting human MHC class I being complexed with HLA—restricted peptide, optionally wherein the HLA restricted peptide is a tumor antigen characterizing the cancer.

An ABP can bind to each portion of an HLA-PEPTIDE complex (i.e., HLA and peptide representing each portion of the complex), which when bound together form a novel target and protein surface for interaction with and binding by the ABP, distinct from a surface presented by the peptide alone or HLA subtype alone. Generally the novel target and protein surface formed by binding of HLA to peptide does not exist in the absence of each portion of the HLA-PEPTIDE complex.

An ABP can be capable of specifically binding a complex comprising HLA and an HLA-restricted peptide (HLA-PEPTIDE), e.g., derived from a tumor. In some aspects, the ABP does not bind HLA in an absence of the HLA-restricted peptide derived from the tumor. In some aspects, the ABP does not bind the HLA-restricted peptide derived from the tumor in an absence of HLA. In some aspects, the ABP binds a complex comprising HLA and HLA-restricted peptide when naturally presented on a cell such as a tumor cell.

In some embodiments, an ABP provided herein modulates binding of HLA-PEPTIDE to one or more ligands of HLA-PEPTIDE.

The ABP may specifically bind to any one of the HLA-PEPTIDE targets as disclosed in Table A. In some embodiments, the ABP specifically binds to a HLA-PEPTIDE target which is a target shown in Table A, with the proviso that the isolated HLA-PEPTIDE target is not any one of Target nos. 6364-6369, 6386-6389, 6500, 6521-6524, or 6578 and is not an HLA-PEPTIDE target found in Table B or Table C. In some embodiments, the HLA-restricted peptide is not from a gene selected from WT1 or MART1.

In more particular embodiments, the ABP specifically binds to an HLA-PEPTIDE target selected from any one of HLA subtype A*02:01 complexed with an HLA-restricted peptide comprising the sequence LLASSILCA, HLA subtype A*01:01 complexed with an HLA-restricted peptide comprising the sequence EVDPIGHLY, HLA subtype B*44:02 complexed with an HLA-restricted peptide comprising the sequence GEMSSNSTAL, HLA subtype A*02:01 complexed with an HLA-restricted peptide comprising the sequence GVYDGEEHSV, HLA subtype *01:01 complexed with an HLA-restricted peptide comprising the sequence EVDPIGHVY, and HLA subtype HLA-A*01:01 complexed with an HLA-restricted peptide comprising the sequence NTDNNLAVY.

In some embodiments, an ABP is an ABP that competes with an illustrative ABP provided herein. In some aspects, the ABP that competes with the illustrative ABP provided herein binds the same epitope as an illustrative ABP provided herein.

In some embodiments, the ABPs described herein are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining ABPs. In some embodiments, a variant is derived from any of the sequences provided herein, wherein one or more conservative amino acid substitutions are made. In some embodiments, a variant is derived from any of the sequences provided herein, wherein one or more nonconservative amino acid substitutions are made. Conservative amino acid substitutions are described herein. Exemplary nonconservative amino acid substitutions include those described in J Immunol. 2008 May 1; 180(9):6116-31, which is hereby incorporated by reference in its entirety. In preferred embodiments, the non-conservative amino acid substitution does not interfere with or inhibit the biological activity of the functional variant. In yet more preferred embodiments, the non-conservative amino acid substitution enhances the biological activity of the functional variant, such that the biological activity of the functional variant is increased as compared to the parent ABP.

ABPs Comprising an Antibody or Antigen-Binding Fragment Thereof

An ABP may comprise an antibody or antigen-binding fragment thereof.

In some embodiments, the ABPs provided herein comprise a light chain. In some aspects, the light chain is a kappa light chain. In some aspects, the light chain is a lambda light chain.

In some embodiments, the ABPs provided herein comprise a heavy chain. In some aspects, the heavy chain is an IgA. In some aspects, the heavy chain is an IgD. In some aspects, the heavy chain is an IgE. In some aspects, the heavy chain is an IgG In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the ABPs provided herein comprise an antibody fragment. In some embodiments, the ABPs provided herein consist of an antibody fragment. In some embodiments, the ABPs provided herein consist essentially of an antibody fragment. In some aspects, the ABP fragment is an Fv fragment. In some aspects, the ABP fragment is a Fab fragment. In some aspects, the ABP fragment is a F(ab′)₂ fragment. In some aspects, the ABP fragment is a Fab′ fragment. In some aspects, the ABP fragment is an scFv (sFv) fragment. In some aspects, the ABP fragment is an scFv-Fc fragment. In some aspects, the ABP fragment is a fragment of a single domain ABP.

In some embodiments, an ABP fragment provided herein is derived from an illustrative ABP provided herein. In some embodiments, an ABP fragments provided herein is not derived from an illustrative ABP provided herein and may, for example, be isolated de novo according to the methods provided herein for obtaining ABP fragments.

In some embodiments, an ABP fragment provided herein retains the ability to bind the HLA-PEPTIDE target, as measured by one or more assays or biological effects described herein. In some embodiments, an ABP fragment provided herein retains the ability to prevent HLA-PEPTIDE from interacting with one or more of its ligands, as described herein.

In some embodiments, the ABPs provided herein are monoclonal ABPs. In some embodiments, the ABPs provided herein are polyclonal ABPs.

In some embodiments, the ABPs provided herein comprise a chimeric ABP. In some embodiments, the ABPs provided herein consist of a chimeric ABP In some embodiments, the ABPs provided herein consist essentially of a chimeric ABP. In some embodiments, the ABPs provided herein comprise a humanized ABP. In some embodiments, the ABPs provided herein consist of a humanized ABP. In some embodiments, the ABPs provided herein consist essentially of a humanized ABP. In some embodiments, the ABPs provided herein comprise a human ABP. In some embodiments, the ABPs provided herein consist of a human ABP In some embodiments, the ABPs provided herein consist essentially of a human ABP.

In some embodiments, the ABPs provided herein comprise an alternative scaffold. In some embodiments, the ABPs provided herein consist of an alternative scaffold. In some embodiments, the ABPs provided herein consist essentially of an alternative scaffold. Any suitable alternative scaffold may be used. In some aspects, the alternative scaffold is selected from an Adnectin™, an iMab, an Anticalin®, an EETI-II/AGRP, a Kunitz domain, a thioredoxin peptide aptamer, an Affibody®, a DARPin, an Affilin, a Tetranectin, a Fynomer, and an Avimer.

Also disclosed herein is an isolated humanized, human, or chimeric ABP that competes for binding to an HLA-PEPTIDE with an ABP disclosed herein.

Also disclosed herein is an isolated humanized, human, or chimeric ABP that binds an HLA-PEPTIDE epitope bound by an ABP disclosed herein.

In certain aspects, an ABP comprises a human Fc region comprising at least one modification that reduces binding to a human Fc receptor.

It is known that when an ABP is expressed in cells, the ABP is modified after translation. Examples of the posttranslational modification include cleavage of lysine at the C terminus of the heavy chain by a carboxypeptidase; modification of glutamine or glutamic acid at the N terminus of the heavy chain and the light chain to pyroglutamic acid by pyroglutamylation; glycosylation; oxidation; deamidation; and glycation, and it is known that such posttranslational modifications occur in various ABPs (See Journal of Pharmaceutical Sciences, 2008, Vol. 97, p. 2426-2447, incorporated by reference in its entirety). In some embodiments, an ABP is an ABP or antigen-binding fragment thereof which has undergone posttranslational modification. Examples of an ABP or antigen-binding fragment thereof which have undergone posttranslational modification include an ABP or antigen-binding fragments thereof which have undergone pyroglutamylation at the N terminus of the heavy chain variable region and/or deletion of lysine at the C terminus of the heavy chain. It is known in the art that such posttranslational modification due to pyroglutamylation at the N terminus and deletion of lysine at the C terminus does not have any influence on the activity of the ABP or fragment thereof (Analytical Biochemistry, 2006, Vol. 348, p. 24-39, incorporated by reference in its entirety).

### Monospecific and Multispecific HLA-PEPTIDE ABPs

In some embodiments, the ABPs provided herein are monospecific ABPs.

In some embodiments, the ABPs provided herein are multispecific ABPs.

In some embodiments, a multispecific ABP provided herein binds more than one antigen. In some embodiments, a multispecific ABP binds 2 antigens. In some embodiments, a multispecific ABP binds 3 antigens. In some embodiments, a multispecific ABP binds 4 antigens. In some embodiments, a multispecific ABP binds 5 antigens.

In some embodiments, a multispecific ABP provided herein binds more than one epitope on a HLA-PEPTIDE antigen. In some embodiments, a multispecific ABP binds 2 epitopes on a HLA-PEPTIDE antigen. In some embodiments, a multispecific ABP binds 3 epitopes on a HLA-PEPTIDE antigen.

Many multispecific ABP constructs are known in the art, and the ABPs provided herein may be provided in the form of any suitable multispecific suitable construct.

In some embodiments, the multispecific ABP comprises an immunoglobulin comprising at least two different heavy chain variable regions each paired with a common light chain variable region (i.e., a “common light chain ABP”). The common light chain variable region forms a distinct antigen-binding domain with each of the two different heavy chain variable regions. See Merchant et al., Nature Biotechnol., 1998, 16:677-681, incorporated by reference in its entirety.

In some embodiments, the multispecific ABP comprises an immunoglobulin comprising an ABP or fragment thereof attached to one or more of the N- or C-termini of the heavy or light chains of such immunoglobulin. See Coloma and Morrison, Nature Biotechnol., 1997, 15:159-163, incorporated by reference in its entirety. In some aspects, such ABP comprises a tetravalent bispecific ABP.

In some embodiments, the multispecific ABP comprises a hybrid immunoglobulin comprising at least two different heavy chain variable regions and at least two different light chain variable regions. See Milstein and Cuello, Nature, 1983, 305:537-540; and Staerz and Bevan, Proc. Natl. Acad. Sci. USA, 1986, 83:1453-1457; each of which is incorporated by reference in its entirety.

In some embodiments, the multispecific ABP comprises immunoglobulin chains with alterations to reduce the formation of side products that do not have multispecificity. In some aspects, the ABPs comprise one or more “knobs-into-holes” modifications as described in U.S. Pat. No. 5,731,168, incorporated by reference in its entirety.

In some embodiments, the multispecific ABP comprises immunoglobulin chains with one or more electrostatic modifications to promote the assembly of Fc hetero-multimers. See WO 2009/089004, incorporated by reference in its entirety.

In some embodiments, the multispecific ABP comprises a bispecific single chain molecule. See Traunecker et al., EMBO J., 1991, 10:3655-3659; and Gruber et al., J. Immunol., 1994, 152:5368-5374; each of which is incorporated by reference in its entirety.

In some embodiments, the multispecific ABP comprises a heavy chain variable domain and a light chain variable domain connected by a polypeptide linker, where the length of the linker is selected to promote assembly of multispecific ABP with the desired multispecificity. For example, monospecific scFvs generally form when a heavy chain variable domain and light chain variable domain are connected by a polypeptide linker of more than 12 amino acid residues. See U.S. Pat. Nos. 4,946,778 and 5,132,405, each of which is incorporated by reference in its entirety. In some embodiments, reduction of the polypeptide linker length to less than 12 amino acid residues prevents pairing of heavy and light chain variable domains on the same polypeptide chain, thereby allowing pairing of heavy and light chain variable domains from one chain with the complementary domains on another chain. The resulting ABP therefore has multispecificity, with the specificity of each binding site contributed by more than one polypeptide chain. Polypeptide chains comprising heavy and light chain variable domains that are joined by linkers between 3 and 12 amino acid residues form predominantly dimers (termed diabodies). With linkers between 0 and 2 amino acid residues, trimers (termed triabodies) and tetramers (termed tetrabodies) are favored. However, the exact type of oligomerization appears to depend on the amino acid residue composition and the order of the variable domain in each polypeptide chain (e.g., V_{H}-linker-V_{L} vs. V_{L}-linker-V_{H}), in addition to the linker length. A skilled person can select the appropriate linker length based on the desired multispecificity.

### Fc Region and Variants

In certain embodiments, an ABP provided herein comprises an Fc region. An Fc region can be wild-type or a variant thereof. In certain embodiments, an ABP provided herein comprises an Fc region with one or more amino acid substitutions, insertions, or deletions in comparison to a naturally occurring Fc region. In some aspects, such substitutions, insertions, or deletions yield ABP with altered stability, glycosylation, or other characteristics. In some aspects, such substitutions, insertions, or deletions yield a glycosylated ABP.

A “variant Fc region” or “engineered Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The term “Fc-region-comprising ABP” refers to an ABP that comprises an Fc region. The C-terminal lysine (residue 447 according to the EU numbering system) of the Fc region may be removed, for example, during purification of the ABP or by recombinant engineering the nucleic acid encoding the ABP. Accordingly, an ABP having an Fc region can comprise an ABP with or without K447.

In some aspects, the Fc region of an ABP provided herein is modified to yield an ABP with altered affinity for an Fc receptor, or an ABP that is more immunologically inert. In some embodiments, the ABP variants provided herein possess some, but not all, effector functions. Such ABPs may be useful, for example, when the half-life of the ABP is important in vivo, but when certain effector functions (e.g., complement activation and ADCC) are unnecessary or deleterious.

In some embodiments, the Fc region of an ABP provided herein is a human IgG4 Fc region comprising one or more of the hinge stabilizing mutations S228P and L235E. See Aalberse et al., Immunology, 2002, 105:9-19, incorporated by reference in its entirety. In some embodiments, the IgG4 Fc region comprises one or more of the following mutations: E233P, F234V, and L235A. See Armour et al., Mol. Immunol., 2003, 40:585-593, incorporated by reference in its entirety. In some embodiments, the IgG4 Fc region comprises a deletion at position G236.

In some embodiments, the Fc region of an ABP provided herein is a human IgG1 Fc region comprising one or more mutations to reduce Fc receptor binding. In some aspects, the one or more mutations are in residues selected from S228 (e.g., S228A), L234 (e.g., L234A), L235 (e.g., L235A), D265 (e.g., D265A), and N297 (e.g., N297A). In some aspects, the ABP comprises a PVA236 mutation. PVA236 means that the amino acid sequence ELLG from amino acid position 233 to 236 of IgG1 or EFLG of IgG4, is replaced by PVA. See U.S. Pat. No. 9,150,641, incorporated by reference in its entirety.

In some embodiments, the Fc region of an ABP provided herein is modified as described in Armour et al., Eur. J. Immunol., 1999, 29:2613-2624; WO 1999/058572; and/or U.K. Pat. App. No. 98099518; each of which is incorporated by reference in its entirety.

In some embodiments, the Fc region of an ABP provided herein is a human IgG2 Fc region comprising one or more of mutations A330S and P331S.

In some embodiments, the Fc region of an ABP provided herein has an amino acid substitution at one or more positions selected from 238, 265, 269, 270, 297, 327 and 329. See U.S. Pat. No. 6,737,056, incorporated by reference in its entirety. Such Fc mutants include Fc mutants with substitutions at two or more of amino acid positions 265, 269, 270, 297 and 327, including the so-called “DANA” Fc mutant with substitution of residues 265 and 297 with alanine. See U.S. Pat. No. 7,332,581, incorporated by reference in its entirety. In some embodiments, the ABP comprises an alanine at amino acid position 265. In some embodiments, the ABP comprises an alanine at amino acid position 297.

In certain embodiments, an ABP provided herein comprises an Fc region with one or more amino acid substitutions which improve ADCC, such as a substitution at one or more of positions 298, 333, and 334 of the Fc region. In some embodiments, an ABP provided herein comprises an Fc region with one or more amino acid substitutions at positions 239, 332, and 330, as described in Lazar et al., Proc. Natl. Acad. Sci. USA, 2006, 103:4005-4010, incorporated by reference in its entirety.

In some embodiments, an ABP provided herein comprises one or more alterations that improves or diminishes C1q binding and/or CDC. See U.S. Pat. No. 6,194,551; WO 99/51642; and Idusogie et al., J. Immunol., 2000, 164:4178-4184; each of which is incorporated by reference in its entirety.

In some embodiments, an ABP provided herein comprises one or more alterations to increase half-life. ABPs with increased half-lives and improved binding to the neonatal Fc receptor (FcRn) are described, for example, in Hinton et al., J. Immunol., 2006, 176:346-356; and U.S. Pat. Pub. No. 2005/0014934; each of which is incorporated by reference in its entirety. Such Fc variants include those with substitutions at one or more of Fc region residues: 238, 250, 256, 265, 272, 286, 303, 305, 307, 311, 312, 314, 317, 340, 356, 360, 362, 376, 378, 380, 382, 413, 424, 428, and 434 of an IgG

In some embodiments, an ABP provided herein comprises one or more Fc region variants as described in U.S. Pat. Nos. 7,371,826 5,648,260, and 5,624,821; Duncan and Winter, Nature, 1988, 322:738-740; and WO 94/29351; each of which is incorporated by reference in its entirety.

Antibodies Specific for A*02:01_LLASSILCA (G7)

In some aspects, provided herein are ABPs comprising antibodies or antigen-binding fragments thereof that specifically bind an HLA-PEPTIDE target, wherein the HLA Class I molecule of the HLA-PEPTIDE target is HLA subtype A*02:01 and the HLA-restricted peptide of the HLA-PEPTIDE target comprises the sequence LLASSILCA (SEQ ID NO: 2737) (“G7”).

Sequences of G7-Specific Antibodies

The ABP specific for A*02:01_LLASSILCA (SEQ ID NO: 2737) may comprise one or more sequences, as described in further detail.

CDRs

The ABP specific for A*02:01_LLASSILCA (SEQ ID NO: 2737) may comprise one or more antibody complementarity determining region (CDR) sequences, e.g., may comprise three heavy chain CDRs (CDR-H1, CDR-H2, CDR-H3) and three light chain CDRs (CDR-L1, CDR-L2, CDR-L3). The ABP specific for A*02:01_LLASSILCA (SEQ ID NO: 2737) may comprise a particular heavy chain CDR3 (CDR-H3) sequence and a particular light chain CDR3 (CDR-L3) sequence. In some embodiments, the CDR-H3 is SEQ ID NO: 3030 and the CDR-L3 is SEQ ID NO: 3048. In some embodiments, the CDR-H3 is SEQ ID NO: 3025 and the CDR-L3 is SEQ ID NO: 3043. In some embodiments, the CDR-H3 is SEQ ID NO: 3026 and the CDR-L3 is SEQ ID NO: 3044. In some embodiments, the CDR-H3 is SEQ ID NO: 3027 and the CDR-L3 is SEQ ID NO: 3045. In some embodiments, the CDR-H3 is SEQ ID NO: 3028 and the CDR-L3 is SEQ ID NO: 3046. In some embodiments, the CDR-H3 is SEQ ID NO: 3029 and the CDR-L3 is SEQ ID NO: 3047. In some embodiments, the CDR-H3 is SEQ ID NO: 3031 and the CDR-L3 is SEQ ID NO: 3049. In some embodiments, the CDR-H3 is SEQ ID NO: 3032 and the CDR-L3 is SEQ ID NO: 3050.

The ABP specific for A*02:01_LLASSILCA (SEQ ID NO: 2737) may comprise a CDR-H1 that is SEQ ID NO: 3010, a CDR-H2 that is SEQ ID NO: 3017, a CDR-H3 that is SEQ ID NO: 3025, a CDR-L1 that is SEQ ID NO: 3033, a CDR-L2 that is SEQ ID NO: 2970, and a CDR-L3 that is SEQ ID NO: 3043. The ABP specific for A*02:01_LLASSILCA (SEQ ID NO: 2737) may comprise a CDR-H1 that is SEQ ID NO: 3011, a CDR-H2 that is SEQ ID NO: 3018, a CDR-H3 that is SEQ ID NO: 3026, a CDR-L1 that is SEQ ID NO: 3034, a CDR-L2 that is SEQ ID NO: 2958, and a CDR-L3 that is SEQ ID NO: 3044. The ABP specific for A*02:01_LLASSILCA (SEQ ID NO: 2737) may comprise a CDR-H1 that is SEQ ID NO: 3012, a CDR-H2 that is SEQ ID NO: 3019, a CDR-H3 that is SEQ ID NO: 3027, a CDR-L1 that is SEQ ID NO: 3035, a CDR-L2 that is SEQ ID NO: 3039, and a CDR-L3 that is SEQ ID NO: 3045. The ABP specific for A*02:01_LLASSILCA (SEQ ID NO: 2737) may comprise a CDR-H1 that is SEQ ID NO: 3013, a CDR-H2 that is SEQ ID NO: 3020, a CDR-H3 that is SEQ ID NO: 3028, a CDR-L1 that is SEQ ID NO: 3036, a CDR-L2 that is SEQ ID NO: 2962, and a CDR-L3 that is SEQ ID NO: 3046. The ABP specific for A*02:01_LLASSILCA (SEQ ID NO: 2737) may comprise a CDR-H1 that is SEQ ID NO: 2879, a CDR-H2 that is SEQ ID NO: 3021, a CDR-H3 that is SEQ ID NO: 3029, a CDR-L1 that is SEQ ID NO: 2934, a CDR-L2 that is SEQ ID NO: 3040, and a CDR-L3 that is SEQ ID NO: 3047. The ABP specific for A*02:01_LLASSILCA (SEQ ID NO: 2737) may comprise a CDR-H1 that is SEQ ID NO: 3014, a CDR-H2 that is SEQ ID NO: 3022, a CDR-H3 that is SEQ ID NO: 3030, a CDR-L1 that is SEQ ID NO: 3037, a CDR-L2 that is SEQ ID NO: 3041, and a CDR-L3 that is SEQ ID NO: 3048. The ABP specific for A*02:01_LLASSILCA (SEQ ID NO: 2737) may comprise a CDR-H1 that is SEQ ID NO: 3015, a CDR-H2 that is SEQ ID NO: 3023, a CDR-H3 that is SEQ ID NO: 3031, a CDR-L1 that is SEQ ID NO: 2946, a CDR-L2 that is SEQ ID NO: 3042, and a CDR-L3 that is SEQ ID NO: 3049. The ABP specific for A*02:01_LLASSILCA (SEQ ID NO: 2737) may comprise a CDR-H1 that is SEQ ID NO: 3016, a CDR-H2 that is SEQ ID NO: 3024, a CDR-H3 that is SEQ ID NO: 3032, a CDR-L1 that is SEQ ID NO: 3038, a CDR-L2 that is SEQ ID NO: 3041, and a CDR-L3 that is SEQ ID NO: 3050.

VL

The ABP specific for *02:01_LLASSILCA (SEQ ID NO: 2737) may comprise a VL sequence. The VL sequence may be SEQ ID NO: 3002. The VL sequence may be SEQ ID NO: 3003. The VL sequence may be SEQ ID NO: 3004. The VL sequence may be SEQ ID NO: 3005. The VL sequence may be SEQ ID NO: 3006. The VL sequence may be SEQ ID NO: 3007. The VL sequence may be SEQ ID NO: 3008. The VL sequence may be SEQ ID NO: 3009.

VH

The ABP specific for *02:01_LLASSILCA (SEQ ID NO: 2737) may comprise a VH sequence. The VH sequence may be SEQ ID NO: 2994. The VH sequence may be SEQ ID NO: 2995. The VH sequence may be SEQ ID NO: 2996. The VH sequence may be SEQ ID NO: 2997. The VH sequence may be SEQ ID NO: 2998. The VH sequence may be SEQ ID NO: 2999. The VH sequence may be SEQ ID NO: 3000. The VH sequence may be SEQ ID NO: 3001.

VH-VL Combinations

The ABP specific for A*02:01_LLASSILCA (SEQ ID NO: 2737) may comprise a VH sequence that is SEQ ID NO: 2994 and a VL sequence that is SEQ ID NO: 3002. The ABP specific for A*02:01_LLASSILCA (SEQ ID NO: 2737) may comprise a VH sequence that is SEQ ID NO: 2995 and a VL sequence that is SEQ ID NO: 3003. The ABP specific for A*02:01_LLASSILCA (SEQ ID NO: 2737) may comprise a VH sequence that is SEQ ID NO: 2996 and a VL sequence that is SEQ ID NO: 3004. The ABP specific for A*02:01 LLASSILCA (SEQ ID NO: 2737) may comprise a VH sequence that is SEQ ID NO: 2997 and a VL sequence that is SEQ ID NO: 3005. The ABP specific for A*02:01_LLASSILCA (SEQ ID NO: 2737) may comprise a VH sequence that is SEQ ID NO: 2998 and a VL sequence that is SEQ ID NO: 3006. The ABP specific for A*02:01_LLASSILCA (SEQ ID NO: 2737) may comprise a VH sequence that is SEQ ID NO: 2999 and a VL sequence that is SEQ ID NO: 3007. The ABP specific for A*02:01_LLASSILCA (SEQ ID NO: 2737) may comprise a VH sequence that is SEQ ID NO: 3000 and a VL sequence that is SEQ ID NO: 3008. The ABP specific for A*02:01_LLASSILCA (SEQ ID NO: 2737) may comprise a VH sequence that is SEQ ID NO: 3001 and a VL sequence that is SEQ ID NO: 3009.

Antibodies Specific for A*01:01_NTDNNLAVY (G2)

In some aspects, provided herein are ABPs comprising antibodies or antigen-binding fragments thereof that specifically bind an HLA-PEPTIDE target, wherein the HLA Class I molecule of the HLA-PEPTIDE target is HLA subtype A*01:01 and the HLA-restricted peptide of the HLA-PEPTIDE target comprises the sequence NTDNNLAVY (SEQ ID NO: 23) (“G2”).

Sequences of G2-Specific Antibodies

The ABP specific for A*01:01_NTDNNLAVY (SEQ ID NO: 23) may comprise one or more sequences, as described in further detail.

CDRs

The ABP specific for A*01:01_NTDNNLAVY (SEQ ID NO: 23) may comprise one or more antibody complementarity determining region (CDR) sequences, e.g., may comprise three heavy chain CDRs (CDR-H1, CDR-H2, CDR-H3) and three light chain CDRs (CDR-L1, CDR-L2, CDR-L3). The ABP specific for A*01:01_NTDNNLAVY (SEQ ID NO: 23) may comprise a particular heavy chain CDR3 (CDR-H3) sequence and a particular light chain CDR3 (CDR-L3) sequence. In some embodiments, the CDR-H3 is SEQ ID NO: 2902 and the CDR-L3 is SEQ ID NO: 2971. In some embodiments, the CDR-H3 is SEQ ID NO: 2903 and the CDR-L3 is SEQ ID NO: 2972. In some embodiments, the CDR-H3 is SEQ ID NO: 2903 and the CDR-L3 is SEQ ID NO: 2973. In some embodiments, the CDR-H3 is SEQ ID NO: 2904 and the CDR-L3 is SEQ ID NO: 2974. In some embodiments, the CDR-H3 is SEQ ID NO: 2905 and the CDR-L3 is SEQ ID NO: 2975. In some embodiments, the CDR-H3 is SEQ ID NO: 2906 and the CDR-L3 is SEQ ID NO: 2976. In some embodiments, the CDR-H3 is SEQ ID NO: 2907 and the CDR-L3 is SEQ ID NO: 2976. In some embodiments, the CDR-H3 is SEQ ID NO: 2908 and the CDR-L3 is SEQ ID NO: 2977. In some embodiments, the CDR-H3 is SEQ ID NO: 2909 and the CDR-L3 is SEQ ID NO: 2972. In some embodiments, the CDR-H3 is SEQ ID NO: 2910 and the CDR-L3 is SEQ ID NO: 2978. In some embodiments, the CDR-H3 is SEQ ID NO: 2911 and the CDR-L3 is SEQ ID NO: 2976. In some embodiments, the CDR-H3 is SEQ ID NO: 2912 and the CDR-L3 is SEQ ID NO: 2978. In some embodiments, the CDR-H3 is SEQ ID NO: 2913 and the CDR-L3 is SEQ ID NO: 2979. In some embodiments, the CDR-H3 is SEQ ID NO: 2914 and the CDR-L3 is SEQ ID NO: 2980. In some embodiments, the CDR-H3 is SEQ ID NO: 2903 and the CDR-L3 is SEQ ID NO: 2981. In some embodiments, the CDR-H3 is SEQ ID NO: 2915 and the CDR-L3 is SEQ ID NO: 2982. In some embodiments, the CDR-H3 is SEQ ID NO: 2916 and the CDR-L3 is SEQ ID NO: 2973. In some embodiments, the CDR-H3 is SEQ ID NO: 2917 and the CDR-L3 is SEQ ID NO: 2972. In some embodiments, the CDR-H3 is SEQ ID NO: 2917 and the CDR-L3 is SEQ ID NO: 2972. In some embodiments, the CDR-H3 is SEQ ID NO: 2918 and the CDR-L3 is SEQ ID NO: 2974. In some embodiments, the CDR-H3 is SEQ ID NO: 2919 and the CDR-L3 is SEQ ID NO: 2983. In some embodiments, the CDR-H3 is SEQ ID NO: 2920 and the CDR-L3 is SEQ ID NO: 2984. In some embodiments, the CDR-H3 is SEQ ID NO: 2921 and the CDR-L3 is SEQ ID NO: 2972. In some embodiments, the CDR-H3 is SEQ ID NO: 2922 and the CDR-L3 is SEQ ID NO: 2985. In some embodiments, the CDR-H3 is SEQ ID NO: 2923 and the CDR-L3 is SEQ ID NO: 2986. In some embodiments, the CDR-H3 is SEQ ID NO: 2924 and the CDR-L3 is SEQ ID NO: 2987. In some embodiments, the CDR-H3 is SEQ ID NO: 2925 and the CDR-L3 is SEQ ID NO: 2973. In some embodiments, the CDR-H3 is SEQ ID NO: 2926 and the CDR-L3 is SEQ ID NO: 2988. In some embodiments, the CDR-H3 is SEQ ID NO: 2927 and the CDR-L3 is SEQ ID NO: 2989. In some embodiments, the CDR-H3 is SEQ ID NO: 2928 and the CDR-L3 is SEQ ID NO: 2981. In some embodiments, the CDR-H3 is SEQ ID NO: 2929 and the CDR-L3 is SEQ ID NO: 2990. In some embodiments, the CDR-H3 is SEQ ID NO: 2930 and the CDR-L3 is SEQ ID NO: 2989. In some embodiments, the CDR-H3 is SEQ ID NO: 2931 and the CDR-L3 is SEQ ID NO: 2991. In some embodiments, the CDR-H3 is SEQ ID NO: 2932 and the CDR-L3 is SEQ ID NO: 2992. In some embodiments, the CDR-H3 is SEQ ID NO: 2933 and the CDR-L3 is SEQ ID NO: 2993.

The ABP specific for A*01:01_NTDNNLAVY (SEQ ID NO: 23) may comprise a CDR-H1 that is SEQ ID NO: 2851, a CDR-H2 that is SEQ ID NO: 2880, a CDR-H3 that is SEQ ID NO: 2902, a CDR-L1 that is SEQ ID NO: 2934, a CDR-L2 that is SEQ ID NO: 2955, and a CDR-L3 that is SEQ ID NO: 2971. The ABP specific for A*01:01_NTDNNLAVY (SEQ ID NO: 23) may comprise a CDR-H1 that is SEQ ID NO: 2852, a CDR-H2 that is SEQ ID NO: 2881, a CDR-H3 that is SEQ ID NO: 2903, a CDR-L1 that is SEQ ID NO: 2935, a CDR-L2 that is SEQ ID NO: 2956, and a CDR-L3 that is SEQ ID NO: 2972. The ABP specific for A*01:01_NTDNNLAVY (SEQ ID NO: 23) may comprise a CDR-H1 that is SEQ ID NO: 2853, a CDR-H2 that is SEQ ID NO: 2882, a CDR-H3 that is SEQ ID NO: 2903, a CDR-L1 that is SEQ ID NO: 2936, a CDR-L2 that is SEQ ID NO: 2957, and a CDR-L3 that is SEQ ID NO: 2973. The ABP specific for A*01:01_NTDNNLAVY (SEQ ID NO: 23) may comprise a CDR-H1 that is SEQ ID NO: 2854, a CDR-H2 that is SEQ ID NO: 2882, a CDR-H3 that is SEQ ID NO: 2904, a CDR-L1 that is SEQ ID NO: 2937, a CDR-L2 that is SEQ ID NO: 2958, and a CDR-L3 that is SEQ ID NO: 2974. The ABP specific for A*01:01_NTDNNLAVY (SEQ ID NO: 23) may comprise a CDR-H1 that is SEQ ID NO: 2855, a CDR-H2 that is SEQ ID NO: 2883, a CDR-H3 that is SEQ ID NO: 2905, a CDR-L1 that is SEQ ID NO: 2937, a CDR-L2 that is SEQ ID NO: 2958, and a CDR-L3 that is SEQ ID NO: 2975. The ABP specific for A*01:01_NTDNNLAVY (SEQ ID NO: 23) may comprise a CDR-H1 that is SEQ ID NO: 2855, a CDR-H2 that is SEQ ID NO: 2882, a CDR-H3 that is SEQ ID NO: 2906, a CDR-L1 that is SEQ ID NO: 2938, a CDR-L2 that is SEQ ID NO: 2958, and a CDR-L3 that is SEQ ID NO: 2976. The ABP specific for A*01:01_NTDNNLAVY (SEQ ID NO: 23) may comprise a CDR-H1 that is SEQ ID NO: 2856, a CDR-H2 that is SEQ ID NO: 2882, a CDR-H3 that is SEQ ID NO: 2907, a CDR-L1 that is SEQ ID NO: 2939, a CDR-L2 that is SEQ ID NO: 2959, and a CDR-L3 that is SEQ ID NO: 2976. The ABP specific for A*01:01_NTDNNLAVY (SEQ ID NO: 23) may comprise a CDR-H1 that is SEQ ID NO: 2857, a CDR-H2 that is SEQ ID NO: 2882, a CDR-H3 that is SEQ ID NO: 2908, a CDR-L1 that is SEQ ID NO: 2940, a CDR-L2 that is SEQ ID NO: 2960, and a CDR-L3 that is SEQ ID NO: 2977. The ABP specific for A*01:01_NTDNNLAVY (SEQ ID NO: 23) may comprise a CDR-H1 that is SEQ ID NO: 2858, a CDR-H2 that is SEQ ID NO: 2884, a CDR-H3 that is SEQ ID NO: 2909, a CDR-L1 that is SEQ ID NO: 2935, a CDR-L2 that is SEQ ID NO: 2958, and a CDR-L3 that is SEQ ID NO: 2972. The ABP specific for A*01:01_NTDNNLAVY (SEQ ID NO: 23) may comprise a CDR-H1 that is SEQ ID NO: 2859, a CDR-H2 that is SEQ ID NO: 2882, a CDR-H3 that is SEQ ID NO: 2910, a CDR-L1 that is SEQ ID NO: 2941, a CDR-L2 that is SEQ ID NO: 2961, and a CDR-L3 that is SEQ ID NO: 2978. The ABP specific for A*01:01_NTDNNLAVY (SEQ ID NO: 23) may comprise a CDR-H1 that is SEQ ID NO: 2852, a CDR-H2 that is SEQ ID NO: 2885, a CDR-H3 that is SEQ ID NO: 2911, a CDR-L1 that is SEQ ID NO: 2942, a CDR-L2 that is SEQ ID NO: 2958, and a CDR-L3 that is SEQ ID NO: 2976. The ABP specific for A*01:01_NTDNNLAVY (SEQ ID NO: 23) may comprise a CDR-H1 that is SEQ ID NO: 2860, a CDR-H2 that is SEQ ID NO: 2882, a CDR-H3 that is SEQ ID NO: 2912, a CDR-L1 that is SEQ ID NO: 2943, a CDR-L2 that is SEQ ID NO: 2962, and a CDR-L3 that is SEQ ID NO: 2978. The ABP specific for A*01:01_NTDNNLAVY (SEQ ID NO: 23) may comprise a CDR-H1 that is SEQ ID NO: 2861, a CDR-H2 that is SEQ ID NO: 2886, a CDR-H3 that is SEQ ID NO: 2913, a CDR-L1 that is SEQ ID NO: 2944, a CDR-L2 that is SEQ ID NO: 2963, and a CDR-L3 that is SEQ ID NO: 2979. The ABP specific for A*01:01_NTDNNLAVY (SEQ ID NO: 23) may comprise a CDR-H1 that is SEQ ID NO: 2862, a CDR-H2 that is SEQ ID NO: 2887, a CDR-H3 that is SEQ ID NO: 2914, a CDR-L1 that is SEQ ID NO: 2945, a CDR-L2 that is SEQ ID NO: 2958, and a CDR-L3 that is SEQ ID NO: 2980. The ABP specific for A*01:01_NTDNNLAVY (SEQ ID NO: 23) may comprise a CDR-H1 that is SEQ ID NO: 2855, a CDR-H2 that is SEQ ID NO: 2888, a CDR-H3 that is SEQ ID NO: 2903, a CDR-L1 that is SEQ ID NO: 2941, a CDR-L2 that is SEQ ID NO: 2962, and a CDR-L3 that is SEQ ID NO: 2981. The ABP specific for A*01:01_NTDNNLAVY (SEQ ID NO: 23) may comprise a CDR-H1 that is SEQ ID NO: 2855, a CDR-H2 that is SEQ ID NO: 2889, a CDR-H3 that is SEQ ID NO: 2915, a CDR-L1 that is SEQ ID NO: 2946, a CDR-L2 that is SEQ ID NO: 2958, and a CDR-L3 that is SEQ ID NO: 2982. The ABP specific for A*01:01_NTDNNLAVY (SEQ ID NO: 23) may comprise a CDR-H1 that is SEQ ID NO: 2863, a CDR-H2 that is SEQ ID NO: 2883, a CDR-H3 that is SEQ ID NO: 2916, a CDR-L1 that is SEQ ID NO: 2947, a CDR-L2 that is SEQ ID NO: 2958, and a CDR-L3 that is SEQ ID NO: 2973. The ABP specific for A*01:01_NTDNNLAVY (SEQ ID NO: 23) may comprise a CDR-H1 that is SEQ ID NO: 2856, a CDR-H2 that is SEQ ID NO: 2890, a CDR-H3 that is SEQ ID NO: 2917, a CDR-L1 that is SEQ ID NO: 2934, a CDR-L2 that is SEQ ID NO: 2962, and a CDR-L3 that is SEQ ID NO: 2972. The ABP specific for A*01:01_NTDNNLAVY (SEQ ID NO: 23) may comprise a CDR-H1 that is SEQ ID NO: 2864, a CDR-H2 that is SEQ ID NO: 2891, a CDR-H3 that is SEQ ID NO: 2917, a CDR-L1 that is SEQ ID NO: 2946, a CDR-L2 that is SEQ ID NO: 2964, and a CDR-L3 that is SEQ ID NO: 2972. The ABP specific for A*01:01_NTDNNLAVY (SEQ ID NO: 23) may comprise a CDR-H1 that is SEQ ID NO: 2865, a CDR-H2 that is SEQ ID NO: 2882, a CDR-H3 that is SEQ ID NO: 2918, a CDR-L1 that is SEQ ID NO: 2941, a CDR-L2 that is SEQ ID NO: 2962, and a CDR-L3 that is SEQ ID NO: 2974. The ABP specific for A*01:01_NTDNNLAVY (SEQ ID NO: 23) may comprise a CDR-H1 that is SEQ ID NO: 2866, a CDR-H2 that is SEQ ID NO: 2882, a CDR-H3 that is SEQ ID NO: 2919, a CDR-L1 that is SEQ ID NO: 2948, a CDR-L2 that is SEQ ID NO: 2958, and a CDR-L3 that is SEQ ID NO: 2983. The ABP specific for A*01:01_NTDNNLAVY (SEQ ID NO: 23) may comprise a CDR-H1 that is SEQ ID NO: 2867, a CDR-H2 that is SEQ ID NO: 2892, a CDR-H3 that is SEQ ID NO: 2920, a CDR-L1 that is SEQ ID NO: 2946, a CDR-L2 that is SEQ ID NO: 2962, and a CDR-L3 that is SEQ ID NO: 2984. The ABP specific for A*01:01_NTDNNLAVY (SEQ ID NO: 23) may comprise a CDR-H1 that is SEQ ID NO: 2868, a CDR-H2 that is SEQ ID NO: 2893, a CDR-H3 that is SEQ ID NO: 2921, a CDR-L1 that is SEQ ID NO: 2949, a CDR-L2 that is SEQ ID NO: 2965, and a CDR-L3 that is SEQ ID NO: 2972. The ABP specific for A*01:01_NTDNNLAVY (SEQ ID NO: 23) may comprise a CDR-H1 that is SEQ ID NO: 2869, a CDR-H2 that is SEQ ID NO: 2894, a CDR-H3 that is SEQ ID NO: 2922, a CDR-L1 that is SEQ ID NO: 2950, a CDR-L2 that is SEQ ID NO: 2966, and a CDR-L3 that is SEQ ID NO: 2985. The ABP specific for A*01:01_NTDNNLAVY (SEQ ID NO: 23) may comprise a CDR-H1 that is SEQ ID NO: 2870, a CDR-H2 that is SEQ ID NO: 2882, a CDR-H3 that is SEQ ID NO: 2923, a CDR-L1 that is SEQ ID NO: 2943, a CDR-L2 that is SEQ ID NO: 2967, and a CDR-L3 that is SEQ ID NO: 2986. The ABP specific for A*01:01_NTDNNLAVY (SEQ ID NO: 23) may comprise a CDR-H1 that is SEQ ID NO: 2871, a CDR-H2 that is SEQ ID NO: 2895, a CDR-H3 that is SEQ ID NO: 2924, a CDR-L1 that is SEQ ID NO: 2951, a CDR-L2 that is SEQ ID NO: 2968, and a CDR-L3 that is SEQ ID NO: 2987. The ABP specific for A*01:01_NTDNNLAVY (SEQ ID NO: 23) may comprise a CDR-H1 that is SEQ ID NO: 2872, a CDR-H2 that is SEQ ID NO: 2882, a CDR-H3 that is SEQ ID NO: 2925, a CDR-L1 that is SEQ ID NO: 2952, a CDR-L2 that is SEQ ID NO: 2969, and a CDR-L3 that is SEQ ID NO: 2973. The ABP specific for A*01:01_NTDNNLAVY (SEQ ID NO: 23) may comprise a CDR-H1 that is SEQ ID NO: 2873, a CDR-H2 that is SEQ ID NO: 2882, a CDR-H3 that is SEQ ID NO: 2926, a CDR-L1 that is SEQ ID NO: 2943, a CDR-L2 that is SEQ ID NO: 2958, and a CDR-L3 that is SEQ ID NO: 2988. The ABP specific for A*01:01_NTDNNLAVY (SEQ ID NO: 23) may comprise a CDR-H1 that is SEQ ID NO: 2852, a CDR-H2 that is SEQ ID NO: 2882, a CDR-H3 that is SEQ ID NO: 2927, a CDR-L1 that is SEQ ID NO: 2935, a CDR-L2 that is SEQ ID NO: 2958, and a CDR-L3 that is SEQ ID NO: 2989. The ABP specific for A*01:01_NTDNNLAVY (SEQ ID NO: 23) may comprise a CDR-H1 that is SEQ ID NO: 2874, a CDR-H2 that is SEQ ID NO: 2896, a CDR-H3 that is SEQ ID NO: 2928, a CDR-L1 that is SEQ ID NO: 2938, a CDR-L2 that is SEQ ID NO: 2958, and a CDR-L3 that is SEQ ID NO: 2981. The ABP specific for A*01:01_NTDNNLAVY (SEQ ID NO: 23) may comprise a CDR-H1 that is SEQ ID NO: 2875, a CDR-H2 that is SEQ ID NO: 2897, a CDR-H3 that is SEQ ID NO: 2929, a CDR-L1 that is SEQ ID NO: 2953, a CDR-L2 that is SEQ ID NO: 2961, and a CDR-L3 that is SEQ ID NO: 2990. The ABP specific for A*01:01_NTDNNLAVY (SEQ ID NO: 23) may comprise a CDR-H1 that is SEQ ID NO: 2876, a CDR-H2 that is SEQ ID NO: 2898, a CDR-H3 that is SEQ ID NO: 2930, a CDR-L1 that is SEQ ID NO: 2941, a CDR-L2 that is SEQ ID NO: 2962, and a CDR-L3 that is SEQ ID NO: 2989. The ABP specific for A*01:01_NTDNNLAVY (SEQ ID NO: 23) may comprise a CDR-H1 that is SEQ ID NO: 2877, a CDR-H2 that is SEQ ID NO: 2899, a CDR-H3 that is SEQ ID NO: 2931, a CDR-L1 that is SEQ ID NO: 2946, a CDR-L2 that is SEQ ID NO: 2964, and a CDR-L3 that is SEQ ID NO: 2991. The ABP specific for A*01:01_NTDNNLAVY (SEQ ID NO: 23) may comprise a CDR-H1 that is SEQ ID NO: 2878, a CDR-H2 that is SEQ ID NO: 2900, a CDR-H3 that is SEQ ID NO: 2932, a CDR-L1 that is SEQ ID NO: 2946, a CDR-L2 that is SEQ ID NO: 2958, and a CDR-L3 that is SEQ ID NO: 2992. The ABP specific for A*01:01_NTDNNLAVY (SEQ ID NO: 23) may comprise a CDR-H1 that is SEQ ID NO: 2879, a CDR-H2 that is SEQ ID NO: 2901, a CDR-H3 that is SEQ ID NO: 2933, a CDR-L1 that is SEQ ID NO: 2954, a CDR-L2 that is SEQ ID NO: 2970, and a CDR-L3 that is SEQ ID NO: 2993.

VL

The ABP specific for A*01:01_NTDNNLAVY (SEQ ID NO: 23) may comprise a VL sequence. The VL sequence may be SEQ ID NO: 2816. The VL sequence may be SEQ ID NO: 2817. The VL sequence may be SEQ ID NO: 2818. The VL sequence may be SEQ ID NO: 2819. The VL sequence may be SEQ ID NO: 2820. The VL sequence may be SEQ ID NO: 2821. The VL sequence may be SEQ ID NO: 2822. The VL sequence may be SEQ ID NO: 2823. The VL sequence may be SEQ ID NO: 2824. The VL sequence may be SEQ ID NO: 2825. The VL sequence may be SEQ ID NO: 2826. The VL sequence may be SEQ ID NO: 2827. The VL sequence may be SEQ ID NO: 2828. The VL sequence may be SEQ ID NO: 2829. The VL sequence may be SEQ ID NO: 2830. The VL sequence may be SEQ ID NO: 2831. The VL sequence may be SEQ ID NO: 2832. The VL sequence may be SEQ ID NO: 2833. The VL sequence may be SEQ ID NO: 2834. The VL sequence may be SEQ ID NO: 2835. The VL sequence may be SEQ ID NO: 2836. The VL sequence may be SEQ ID NO: 2837. The VL sequence may be SEQ ID NO: 2838. The VL sequence may be SEQ ID NO: 2839. The VL sequence may be SEQ ID NO: 2840. The VL sequence may be SEQ ID NO: 2841. The VL sequence may be SEQ ID NO: 2842. The VL sequence may be SEQ ID NO: 2843. The VL sequence may be SEQ ID NO: 2844. The VL sequence may be SEQ ID NO: 2845. The VL sequence may be SEQ ID NO: 2846. The VL sequence may be SEQ ID NO: 2847. The VL sequence may be SEQ ID NO: 2848. The VL sequence may be SEQ ID NO: 2849. The VL sequence may be SEQ ID NO: 2850.

VH

The ABP specific for A*01:01_NTDNNLAVY (SEQ ID NO: 23) may comprise a VH sequence. The VH sequence may be SEQ ID NO: 2781. The VH sequence may be SEQ ID NO: 2782. The VH sequence may be SEQ ID NO: 2783. The VH sequence may be SEQ ID NO: 2784. The VH sequence may be SEQ ID NO: 2785. The VH sequence may be SEQ ID NO: 2786. The VH sequence may be SEQ ID NO: 2787. The VH sequence may be SEQ ID NO: 2788. The VH sequence may be SEQ ID NO: 2789. The VH sequence may be SEQ ID NO: 2790. The VH sequence may be SEQ ID NO: 2791. The VH sequence may be SEQ ID NO: 2792. The VH sequence may be SEQ ID NO: 2793. The VH sequence may be SEQ ID NO: 2794. The VH sequence may be SEQ ID NO: 2795. The VH sequence may be SEQ ID NO: 2796. The VH sequence may be SEQ ID NO: 2797. The VH sequence may be SEQ ID NO: 2798. The VH sequence may be SEQ ID NO: 2799. The VH sequence may be SEQ ID NO: 2800. The VH sequence may be SEQ ID NO: 2801. The VH sequence may be SEQ ID NO: 2802. The VH sequence may be SEQ ID NO: 2803. The VH sequence may be SEQ ID NO: 2804. The VH sequence may be SEQ ID NO: 2805. The VH sequence may be SEQ ID NO: 2806. The VH sequence may be SEQ ID NO: 2807. The VH sequence may be SEQ ID NO: 2808. The VH sequence may be SEQ ID NO: 2809. The VH sequence may be SEQ ID NO: 2810. The VH sequence may be SEQ ID NO: 2811. The VH sequence may be SEQ ID NO: 2812. The VH sequence may be SEQ ID NO: 2813. The VH sequence may be SEQ ID NO: 2814. The VH sequence may be SEQ ID NO: 2815.

VH-VL Combinations

The ABP specific for A*01:01_NTDNNLAVY (SEQ ID NO: 23) may comprise a VH sequence that is SEQ ID NO: 2781 and a VL sequence that is SEQ ID NO: 2816. The ABP specific for A*01:01_NTDNNLAVY (SEQ ID NO: 23) may comprise a VH sequence that is SEQ ID NO: 2782 and a VL sequence that is SEQ ID NO: 2817. The ABP specific for A*01:01_NTDNNLAVY (SEQ ID NO: 23) may comprise a VH sequence that is SEQ ID NO: 2783 and a VL sequence that is SEQ ID NO: 2818. The ABP specific for A*01:01_NTDNNLAVY (SEQ ID NO: 23) may comprise a VH sequence that is SEQ ID NO: 2784 and a VL sequence that is SEQ ID NO: 2819. The ABP specific for A*01:01_NTDNNLAVY (SEQ ID NO: 23) may comprise a VH sequence that is SEQ ID NO: 2785 and a VL sequence that is SEQ ID NO: 2820. The ABP specific for A*01:01_NTDNNLAVY (SEQ ID NO: 23) may comprise a VH sequence that is SEQ ID NO: 2786 and a VL sequence that is SEQ ID NO: 2821. The ABP specific for A*01:01_NTDNNLAVY (SEQ ID NO: 23) may comprise a VH sequence that is SEQ ID NO: 2787 and a VL sequence that is SEQ ID NO: 2822. The ABP specific for A*01:01_NTDNNLAVY (SEQ ID NO: 23) may comprise a VH sequence that is SEQ ID NO: 2788 and a VL sequence that is SEQ ID NO: 2823. The ABP specific for A*01:01_NTDNNLAVY (SEQ ID NO: 23) may comprise a VH sequence that is SEQ ID NO: 2789 and a VL sequence that is SEQ ID NO: 2824. The ABP specific for A*01:01_NTDNNLAVY (SEQ ID NO: 23) may comprise a VH sequence that is SEQ ID NO: 2790 and a VL sequence that is SEQ ID NO: 2825. The ABP specific for A*01:01_NTDNNLAVY (SEQ ID NO: 23) may comprise a VH sequence that is SEQ ID NO: 2791 and a VL sequence that is SEQ ID NO: 2826. The ABP specific for A*01:01_NTDNNLAVY (SEQ ID NO: 23) may comprise a VH sequence that is SEQ ID NO: 2792 and a VL sequence that is SEQ ID NO: 2827. The ABP specific for A*01:01_NTDNNLAVY (SEQ ID NO: 23) may comprise a VH sequence that is SEQ ID NO: 2793 and a VL sequence that is SEQ ID NO: 2828. The ABP specific for A*01:01_NTDNNLAVY (SEQ ID NO: 23) may comprise a VH sequence that is SEQ ID NO: 2794 and a VL sequence that is SEQ ID NO: 2829. The ABP specific for A*01:01_NTDNNLAVY (SEQ ID NO: 23) may comprise a VH sequence that is SEQ ID NO: 2795 and a VL sequence that is SEQ ID NO: 2830. The ABP specific for A*01:01_NTDNNLAVY (SEQ ID NO: 23) may comprise a VH sequence that is SEQ ID NO: 2796 and a VL sequence that is SEQ ID NO: 2831. The ABP specific for A*01:01_NTDNNLAVY (SEQ ID NO: 23) may comprise a VH sequence that is SEQ ID NO: 2797 and a VL sequence that is SEQ ID NO: 2832. The ABP specific for A*01:01_NTDNNLAVY (SEQ ID NO: 23) may comprise a VH sequence that is SEQ ID NO: 2798 and a VL sequence that is SEQ ID NO: 2833. The ABP specific for A*01:01_NTDNNLAVY (SEQ ID NO: 23) may comprise a VH sequence that is SEQ ID NO: 2799 and a VL sequence that is SEQ ID NO: 2834. The ABP specific for A*01:01_NTDNNLAVY (SEQ ID NO: 23) may comprise a VH sequence that is SEQ ID NO: 2800 and a VL sequence that is SEQ ID NO: 2835. The ABP specific for A*01:01_NTDNNLAVY (SEQ ID NO: 23) may comprise a VH sequence that is SEQ ID NO: 2801 and a VL sequence that is SEQ ID NO: 2836. The ABP specific for A*01:01_NTDNNLAVY (SEQ ID NO: 23) may comprise a VH sequence that is SEQ ID NO: 2802 and a VL sequence that is SEQ ID NO: 2837. The ABP specific for A*01:01_NTDNNLAVY (SEQ ID NO: 23) may comprise a VH sequence that is SEQ ID NO: 2803 and a VL sequence that is SEQ ID NO: 2838. The ABP specific for A*01:01_NTDNNLAVY (SEQ ID NO: 23) may comprise a VH sequence that is SEQ ID NO: 2804 and a VL sequence that is SEQ ID NO: 2839. The ABP specific for A*01:01_NTDNNLAVY (SEQ ID NO: 23) may comprise a VH sequence that is SEQ ID NO: 2805 and a VL sequence that is SEQ ID NO: 2840. The ABP specific for A*01:01_NTDNNLAVY (SEQ ID NO: 23) may comprise a VH sequence that is SEQ ID NO: 2806 and a VL sequence that is SEQ ID NO: 2841. The ABP specific for A*01:01_NTDNNLAVY (SEQ ID NO: 23) may comprise a VH sequence that is SEQ ID NO: 2807 and a VL sequence that is SEQ ID NO: 2842. The ABP specific for A*01:01_NTDNNLAVY (SEQ ID NO: 23) may comprise a VH sequence that is SEQ ID NO: 2808 and a VL sequence that is SEQ ID NO: 2843. The ABP specific for A*01:01_NTDNNLAVY (SEQ ID NO: 23) may comprise a VH sequence that is SEQ ID NO: 2809 and a VL sequence that is SEQ ID NO: 2844. The ABP specific for A*01:01_NTDNNLAVY (SEQ ID NO: 23) may comprise a VH sequence that is SEQ ID NO: 2810 and a VL sequence that is SEQ ID NO: 2845. The ABP specific for A*01:01_NTDNNLAVY (SEQ ID NO: 23) may comprise a VH sequence that is SEQ ID NO: 2811 and a VL sequence that is SEQ ID NO: 2846. The ABP specific for A*01:01_NTDNNLAVY (SEQ ID NO: 23) may comprise a VH sequence that is SEQ ID NO: 2812 and a VL sequence that is SEQ ID NO: 2847. The ABP specific for A*01:01_NTDNNLAVY (SEQ ID NO: 23) may comprise a VH sequence that is SEQ ID NO: 2813 and a VL sequence that is SEQ ID NO: 2848. The ABP specific for A*01:01_NTDNNLAVY (SEQ ID NO: 23) may comprise a VH sequence that is SEQ ID NO: 2814 and a VL sequence that is SEQ ID NO: 2849. The ABP specific for A*01:01_NTDNNLAVY (SEQ ID NO: 23) may comprise a VH sequence that is SEQ ID NO: 2815 and a VL sequence that is SEQ ID NO: 2850.

Receptors

Among the provided ABPs, e.g., HLA-PEPTIDE ABPs, are receptors. The receptors can include antigen receptors and other chimeric receptors that specifically bind an HLA-PEPTIDE target disclosed herein. The receptor may be a T cell receptor (TCR). The receptor may be a chimeric antigen receptor (CAR).

TCRs can be soluble or membrane-bound. Among the antigen receptors are functional non-TCR antigen receptors, such as chimeric antigen receptors (CARs). Also provided are cells expressing the receptors and uses thereof in adoptive cell therapy, such as treatment of diseases and disorders associated with HLA-PEPTIDE expression, including cancer.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Exemplary of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, e.g., and in which the antigen-binding portion, e.g., scFv, is replaced by an antibody, e.g., as provided herein.

Among the chimeric receptors are chimeric antigen receptors (CARs). The chimeric receptors, such as CARs, generally include an extracellular antigen binding domain that includes, is, or is comprised within, one of the provided anti-HLA-PEPTIDE ABPs such as anti-HLA-PEPTIDE antibodies. Thus, the chimeric receptors, e.g., CARs, typically include in their extracellular portions one or more HLA-PEPTIDE-ABPs, such as one or more antigen-binding fragment, domain, or portion, or one or more antibody variable domains, and/or antibody molecules, such as those described herein. In some embodiments, the CAR includes a HLA-PEPTIDE-binding portion or portions of the ABP (e.g., antibody) molecule, such as a variable heavy (VH) chain region and/or variable light (VL) chain region of the antibody, e.g., an scFv antibody fragment.

TCRs

In an aspect, the ABPs provided herein, e.g., ABPs that specifically bind HLA-PEPTIDE targets disclosed herein, include T cell receptors (TCRs). The TCRs may be isolated and purified.

In a majority of T-cells, the TCR is a heterodimer polypeptide having an alpha (α) chain and beta-(β) chain, encoded by TRA and TRB, respectively. The alpha chain generally comprises an alpha variable region, encoded by TRAV, an alpha joining region, encoded by TRAJ, and an alpha constant region, encoded by TRAC. The beta chain generally comprises a beta variable region, encoded by TRBV, a beta diversity region, encoded by TRBD, a beta joining region, encoded by TRBJ, and a beta constant region, encoded by TRBC. The TCR-alpha chain is generated by VJ recombination, and the beta chain receptor is generated by V(D)J recombination. Additional TCR diversity stems from junctional diversity. Several bases may be deleted and others added (called N and P nucleotides) at each of the junctions. In a minority of T-cells, the TCRs include gamma and delta chains. The TCR gamma chain is generated by VJ recombination, and the TCR delta chain is generated by V(D)J recombination (Kenneth Murphy, Paul Travers, and Mark Walport, Janeway's Immunology 7th edition, Garland Science, 2007, which is herein incorporated by reference in its entirety). The antigen binding site of a TCR generally comprises six complementarity determining regions (CDRs). The alpha chain contributes three CDRs, alpha CDR1, alpha CDR2, and αCDR3. The beta chain also contributes three CDR: beta CDR1, beta CDR2, and βCDR3. The αCDR3 and βCDR3 are the regions most affected by V(D)J recombination and account for most of the variation in a TCR repertoire.

TCRs can specifically recognize HLA-PEPTIDE targets, such as an HLA-PEPTIDE target disclosed in Table A; thus TCRs can be ABPs that specifically bind to HLA-PEPTIDE. TCRs can be soluble, e.g., similar to an antibody secreted by a B cell. TCRs can also be membrane-bound, e.g., on a cell such as a T cell or NK cell. Thus, TCRs can be used in a context that corresponds to soluble antibodies and/or membrane-bound CARs.

Any of the TCRs disclosed herein may comprise an alpha variable region, an alpha joining region, optionally an alpha constant region, a beta variable region, optionally a beta diversity region, a beta joining region, and optionally a beta constant region.

In some embodiments, the TCR or CAR is a recombinant TCR or CAR. The recombinant TCR or CAR may include any of the TCRs identified herein but include one or more modifications. Exemplary modifications, e.g., amino acid substitutions, are described herein. Amino acid substitutions described herein may be made with reference to IMGT nomenclature and amino acid numbering as found at www.imgt.org.

The recombinant TCR or CAR may be a human TCR or CAR, comprising fully human sequences, e.g., natural human sequences. The recombinant TCR or CAR may retain its natural human variable domain sequences but contain modifications to the α constant region, β constant region, or both α and β constant regions. Such modifications to the TCR constant regions may improve TCR assembly and expression for TCR gene therapy by, e.g., driving preferential pairings of the exogenous TCR chains.

In some embodiments, the α and β constant regions are modified by substituting the entire human constant region sequences for mouse constant region sequences. Such “murinized” TCRs and methods of making them are described in Cancer Res. 2006 Sep. 1; 66(17):8878-86, which is hereby incorporated by reference in its entirety.

In some embodiments, the α and β constant regions are modified by making one or more amino acid substitutions in the human TCR α constant (TRAC) region, the TCR β constant (TRBC) region, or the TRAC and TRAB regions, which swap particular human residues for murine residues (human [symbol] murine amino acid exchange). The one or more amino acid substitutions in the TRAC region may include a Ser substitution at residue 90, an Asp substitution at residue 91, a Val substitution at residue 92, a Pro substitution at residue 93, or any combination thereof. The one or more amino acid substitutions in the human TRBC region may include a Lys substitution at residue 18, an Ala substitution at residue 22, an Ile substitution at residue 133, a His substitution at residue 139, or any combination of the above. Such targeted amino acid substitutions are described in J Immunol Jun. 1, 2010, 184 (11) 6223-6231, which is hereby incorporated by reference in its entirety.

In some embodiments, the human TRAC contains an Asp substitution at residue 210 and the human TRBC contains a Lys substitution at residue 134. Such substitutions may promote the formation of a salt bridge between the alpha and beta chains and formation of the TCR interchain disulfide bond. These targeted substitutions are described in J Immunol Jun. 1, 2010, 184 (11) 6232-6241, which is hereby incorporated by reference in its entirety.

In some embodiments, the human TRAC and human TRBC regions are modified to contain introduced cysteines which may improve preferential pairing of the exogenous TCR chains through formation of an additional disulfide bond. For example, the human TRAC may contain a Cys substitution at residue 48 and the human TRBC may contain a Cys substitution at residue 57, described in Cancer Res. 2007 Apr. 15; 67(8):3898-903 and Blood. 2007 Mar. 15; 109(6):2331-8, which are hereby incorporated by reference in their entirety.

The recombinant TCR or CAR may comprise other modifications to the α and β chains.

In some embodiments, the α and β chains are modified by linking the extracellular domains of the α and β chains to a complete human CD3E (CD3-zeta) molecule. Such modifications are described in J Immunol Jun. 1, 2008, 180 (11) 7736-7746; Gene Ther. 2000 August; 7(16):1369-77; and The Open Gene Therapy Journal, 2011, 4: 11-22, which are hereby incorporated by reference in their entirety.

In some embodiments, the α chain is modified by introducing hydrophobic amino acid substitutions in the transmembrane region of the α chain, as described in J Immunol Jun. 1, 2012, 188 (11) 5538-5546; hereby incorporated by reference in their entirety.

The alpha or beta chain may be modified by altering any one of the N-glycosylation sites in the amino acid sequence, as described in J Exp Med. 2009 Feb. 16; 206(2): 463-475; hereby incorporated by reference in its entirety.

The alpha and beta chain may each comprise a dimerization domain, e.g., a heterologous dimerization domain. Such a heterologous domain may be a leucine zipper, a 5H3 domain or hydrophobic proline rich counter domains, or other similar modalities, as known in the art. In one example, the alpha and beta chains may be modified by introducing 30mer segments to the carboxyl termini of the alpha and beta extracellular domains, wherein the segments selectively associate to form a stable leucine zipper. Such modifications are described in PNAS Nov. 22, 1994. 91 (24) 11408-11412; https://doi.org/10.1073/pnas.91.24.11408; hereby incorporated by reference in its entirety.

TCRs identified herein may be modified to include mutations that result in increased affinity or half-life, such as those described in WO2012/013913, hereby incorporated by reference in its entirety.

The recombinant TCR or CAR may be a single chain TCR (scTCR). Such scTCR may comprise an α chain variable region sequence fused to the N terminus of a TCR α chain constant region extracellular sequence, a TCR β chain variable region fused to the N terminus of a TCR β chain constant region extracellular sequence, and a linker sequence linking the C terminus of the α segment to the N terminus of the β segment, or vice versa. In some embodiments, the constant region extracellular sequences of the α and β segments of the scTCR are linked by a disulfide bond. In some embodiments, the length of the linker sequence and the position of the disulfide bond being such that the variable region sequences of the α and β segments are mutually orientated substantially as in native αβ T cell receptors. Exemplary scTCRs are described in U.S. Pat. No. 7,569,664, which is hereby incorporated by reference in its entirety.

In some cases, the variable regions of the scTCR may be covalently joined by a short peptide linker, such as described in Gene Therapy volume 7, pages 1369-1377 (2000). The short peptide linker may be a serine rich or glycine rich linker. For example, the linker may be (Gly₄Ser)₃, as described in Cancer Gene Therapy (2004) 11, 487-496, incorporated by reference in its entirety.

The recombinant TCR or antigen binding fragment thereof may be expressed as a fusion protein. For instance, the TCR or antigen binding fragment thereof may be fused with a toxin. Such fusion proteins are described in Cancer Res. 2002 Mar. 15; 62(6):1757-60. The TCR or antigen binding fragment thereof may be fused with an antibody Fc region. Such fusion proteins are described in J Immunol May 1, 2017, 198 (1 Supplement) 120.9.

In some embodiments, the recombinant receptor such as a TCR or CAR, such as the antibody portion thereof, further includes a spacer, which may be or include at least a portion of an immunoglobulin constant region or variant or modified version thereof, such as a hinge region, e.g., an IgG4 hinge region, and/or a CH1/CL and/or Fc region. In some embodiments, the constant region or portion is of a human IgG such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014031687. In some embodiments, the constant region or portion is of IgD.

The antigen recognition domain of a receptor such as a TCR or CAR can be linked to one or more intracellular signaling components, such as signaling components that mimic activation through an antigen receptor complex, such as a TCR complex, in the case of a CAR, and/or signal via another cell surface receptor. Thus, in some embodiments, the HLA-PEPTIDE-specific binding component (e.g., ABP such as antibody or TCR) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, and/or CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

The receptor, e.g., the TCR or CAR, can include at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the HLA-PEPTIDE-binding ABP (e.g., antibody) is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor-gamma, CD8, CD4, CD25, or CD16. For example, in some aspects, the CAR includes a chimeric molecule between CD3-zeta or Fc receptor-gamma and CD8, CD4, CD25 or CD16.

In some embodiments, upon ligation of the TCR or CAR, the cytoplasmic domain or intracellular signaling domain of the receptor activates at least one of the normal effector functions or responses of the immune cell, e.g., T cell engineered to express the receptor. For example, in some contexts, the receptor induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the receptor. In other embodiments, the receptor does not include a component for generating a costimulatory signal. In some aspects, an additional receptor is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the receptor includes one or both of such signaling components.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components.

In some embodiments, the activating domain is included within one receptor, whereas the costimulatory component is provided by another receptor recognizing another antigen. In some embodiments, the receptors include activating or stimulatory receptors, and costimulatory receptors, both expressed on the same cell (see WO2014/055668). In some aspects, the HLA-PEPTIDE-targeting receptor is the stimulatory or activating receptor; in other aspects, it is the costimulatory receptor. In some embodiments, the cells further include inhibitory receptors (e.g., iCARs, see Fedorov et al., Sci. Transl. Medicine, 5(215) (December, 2013), such as a receptor recognizing an antigen other than HLA-PEPTIDE, whereby an activating signal delivered through the HLA-PEPTIDE-targeting receptor is diminished or inhibited by binding of the inhibitory receptor to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the receptor encompasses one or more, e.g., two or more, costimulatory domains and an activation domain, e.g., primary activation domain, in the cytoplasmic portion. Exemplary receptors include intracellular components of CD3-zeta, CD28, and 4-1BB.

In some embodiments, the CAR or other antigen receptor such as a TCR further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence or a ribosomal skip sequence, e.g., T2A. See WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published patent application No. WO2014031687. For example, the marker can be a truncated EGFR (tEGFR) that is, optionally, linked to a linker sequence, such as a T2A ribosomal skip sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

The TCR or CAR may comprise one or modified synthetic amino acids in place of one or more naturally-occurring amino acids. Exemplary modified amino acids include, but are not limited to, aminocyclohexane carboxylic acid, norleucine, α-amino n-decanoic acid, homoserine, S-acetylaminomethylcysteine, trans-3- and trans-4-hydroxyproline, 4-aminophenylalanine, 4-nitrophenylalanine, 4-chlorophenylalanine, 4-carboxyphenylalanine, (3-phenylserine (3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,γ-diaminopropionic acid, homophenylalanine, and α-tertbutylglycine.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular portion containing an antibody or fragment described herein. In some aspects, the chimeric antigen receptor includes an extracellular portion containing an antibody or fragment described herein and an intracellular signaling domain. In some embodiments, an antibody or fragment includes an scFv or a single-domain VH antibody and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR, is a transmembrane domain of human CD28 or variant thereof, e.g., a 27-amino acid transmembrane domain of a human CD28 (Accession No.: P10747.1).

In some embodiments, the chimeric antigen receptor contains an intracellular domain of a T cell costimulatory molecule. In some aspects, the T cell costimulatory molecule is CD28 or 41BB.

In some embodiments, the intracellular signaling domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular domain comprises an intracellular costimulatory signaling domain of 41BB or functional variant or portion thereof, such as a 42-amino acid cytoplasmic domain of a human 4-1BB (Accession No. Q07011.1) or functional variant or portion thereof.

In some embodiments, the intracellular signaling domain comprises a human CD3 zeta stimulatory signaling domain or functional variant thereof, such as a 112 AA cytoplasmic domain of isoform 3 of human CD3.zeta. (Accession No.: P20963.2) or a CD3 zeta signaling domain as described in U.S. Pat. No. 7,446,190 or 8,911,993.

In some aspects, the spacer contains only a hinge region of an IgG, such as only a hinge of IgG4 or IgG1. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, the CAR includes an antibody or fragment thereof, such as any of the HLA-PEPTIDE antibodies, including sdAbs (e.g. containing only the VH region) and scFvs, described herein, a spacer such as any of the Ig-hinge containing spacers, a CD28 transmembrane domain, a CD28 intracellular signaling domain, and a CD3 zeta signaling domain. In some embodiments, the CAR includes an antibody or fragment, such as any of the HLA-PEPTIDE antibodies, including sdAbs and scFvs described herein, a spacer such as any of the Ig-hinge containing spacers, a CD28 transmembrane domain, a CD28 intracellular signaling domain, and a CD3 zeta signaling domain.

Target-Specific TCRs to a *02:01 LLASSILCA (SEQ ID NO: 2737) [G7]

In some aspects, provided herein are ABPs comprising TCRs or antigen-binding fragments thereof that specifically bind an HLA-PEPTIDE target, wherein the HLA Class I molecule of the HLA-PEPTIDE target is HLA subtype A*02:01 and the HLA-restricted peptide of the HLA-PEPTIDE target comprises the sequence LLASSILCA (SEQ ID NO: 2737) (“G7”).

The TCR specific for A*02:01_LLASSILCA (SEQ ID NO: 2737) may comprise an αCDR3 sequence. The αCDR3 sequence may be SEQ ID NO: 4277, 4278, 4279, 4280, or 4281.

The TCR specific for A*02:01_LLASSILCA (SEQ ID NO: 2737) may comprise a βCDR3 sequence. The βCDR3 sequence may be any one of SEQ ID NOS 4291-4295.

The TCR specific for A*02:01_LLASSILCA (SEQ ID NO: 2737) may comprise a particular αCDR3 sequence and a particular βCDR3 sequence. The αCDR3 may be SEQ ID NO: 4277 and the βCDR3 may be SEQ ID NO: 4291. The αCDR3 may be SEQ ID NO: 4278 and the βCDR3 may be SEQ ID NO: 4292. The αCDR3 may be SEQ ID NO: 4279 and the βCDR3 may be SEQ ID NO: 4293. The αCDR3 may be SEQ ID NO: 4280 and the βCDR3 may be SEQ ID NO: 4294. The αCDR3 may be SEQ ID NO: 4281 and the βCDR3 may be SEQ ID NO: 4295.

The TCR specific for A*02:01_LLASSILCA (SEQ ID NO: 2737) may comprise an αCDR3 that is SEQ ID NO: 4277 and a beta CDR 3 that is SEQ ID NO: 4291. The TCR specific for A*02:01_LLASSILCA (SEQ ID NO: 2737) may comprise an αCDR3 that is SEQ ID NO: 4278 and a beta CDR 3 that is SEQ ID NO: 4292. The TCR specific for A*02:01_LLASSILCA (SEQ ID NO: 2737) may comprise an αCDR3 that is SEQ ID NO: 4279 and a beta CDR 3 that is SEQ ID NO: 4293. The TCR specific for A*02:01_LLASSILCA (SEQ ID NO: 2737) may comprise an αCDR3 that is SEQ ID NO: 4280 and a beta CDR 3 that is SEQ ID NO: 4294. The TCR specific for A*02:01_LLASSILCA (SEQ ID NO: 2737) may comprise an αCDR3 that is SEQ ID NO: 4281 and a beta CDR 3 that is SEQ ID NO: 4295.

The TCR specific for A*02:01_LLASSILCA (SEQ ID NO: 2737) may comprise a TRAV, a TRAJ, a TRBV, optionally a TRBD, and a TRBJ amino acid sequence, optionally a TRAC sequence and optionally a TRBC sequence. Such TCR may comprise TRAV19, TRAJ4, TRBV6-5, TRBD2, and TRBJ2-7. Such TCR may comprise TRAV5, TRAJ13, TRBV7-9, TRBD1, and TRBJ2-7. Such TCR may comprise TRAV3, TRAJ39, TRBV7-9, and TRBJ2-2. Such TCR may comprise TRAV38-2DV8, TRAJ21, TRBV9, TRBD1, and TRBJ2-1. Such TCR may comprise TRAV4, TRAJ9, TRBV27, and TRBJ1-5.

The TCR specific for A*02:01_LLASSILCA (SEQ ID NO: 2737) may comprise an alpha VJ sequence. The alpha VJ sequence may be any one of SEQ ID NOS 4306-4310.

The TCR specific for A*02:01_LLASSILCA (SEQ ID NO: 2737) may comprise a beta V(D)J sequence. The beta V(D)J sequence may be any one of SEQ ID NOS 4321-4325.

In some embodiments, the alpha VJ sequence is SEQ ID NO: 4306 and the beta V(D)J sequence is SEQ ID NO: 4321. In some embodiments, the alpha VJ sequence is SEQ ID NO: 4307 and the beta V(D)J sequence is SEQ ID NO: 4322. In some embodiments, the alpha VJ sequence is SEQ ID NO: 4308 and the beta V(D)J sequence is SEQ ID NO: 4323. In some embodiments, the alpha VJ sequence is SEQ ID NO: 4309 and the beta V(D)J sequence is SEQ ID NO: 4324. In some embodiments, the alpha VJ sequence is SEQ ID NO: 4310 and the beta V(D)J sequence is SEQ ID NO: 4325.

Target-Specific TCRs to A*01:01 EVDPIGHLY (SEQ ID NO: 3051)

In some aspects, provided herein are ABPs comprising TCRs or antigen-binding fragments thereof that specifically bind an HLA-PEPTIDE target, wherein the HLA Class I molecule of the HLA-PEPTIDE target is HLA subtype A*01:01 and the HLA-restricted peptide of the HLA-PEPTIDE target comprises the sequence EVDPIGHLY (SEQ ID NO: 3051).

The TCR specific for A*01:01_EVDPIGHLY (SEQ ID NO: 3051) may comprise an αCDR3 sequence. The αCDR3 sequence may be any one of SEQ ID NOS 3052-3350 or 4273-4276.

The TCR specific for A*01:01_EVDPIGHLY (SEQ ID NO: 3051) may comprise a βCDR3 sequence. The βCDR3 sequence may be any one of SEQ ID NOS 3351-3655 or 4287-4290.

The TCR specific for A*01:01_EVDPIGHLY (SEQ ID NO: 3051) may comprise a particular αCDR3 sequence and a particular βCDR3 sequence. The αCDR3 may be SEQ ID NO: 4273 and the βCDR3 may be SEQ ID NO: 4287. The αCDR3 may be SEQ ID NO: 4274 and the βCDR3 may be SEQ ID NO: 4288. The αCDR3 may be SEQ ID NO: 4275 and the βCDR3 may be SEQ ID NO: 4289. The αCDR3 may be SEQ ID NO: 4276 and the βCDR3 may be SEQ ID NO: 4290. The αCDR3 may be SEQ ID NO: 3052 and the βCDR3 may be SEQ ID NO: 3351. The αCDR3 may be SEQ ID NO: 3053 and the βCDR3 may be SEQ ID NO: 3352. The αCDR3 may be SEQ ID NO: 3054 and the βCDR3 may be SEQ ID NO: 3353. The αCDR3 may be SEQ ID NO: 3052 and the βCDR3 may be SEQ ID NO: 3352. The αCDR3 may be SEQ ID NO: 3055 and the βCDR3 may be SEQ ID NO: 3354. The αCDR3 may be SEQ ID NO: 3056 and the βCDR3 may be SEQ ID NO: 3355. The αCDR3 may be SEQ ID NO: 3057 and the βCDR3 may be SEQ ID NO: 3356. The αCDR3 may be SEQ ID NO: 3058 and the βCDR3 may be SEQ ID NO: 3357. The αCDR3 may be SEQ ID NO: 3059 and the βCDR3 may be SEQ ID NO: 3358. The αCDR3 may be SEQ ID NO: 3060 and the βCDR3 may be SEQ ID NO: 3359. The αCDR3 may be SEQ ID NO: 3061 and the βCDR3 may be SEQ ID NO: 3360. The αCDR3 may be SEQ ID NO: 3062 and the βCDR3 may be SEQ ID NO: 3361. The αCDR3 may be SEQ ID NO: 3063 and the βCDR3 may be SEQ ID NO: 3362. The αCDR3 may be SEQ ID NO: 3053 and the βCDR3 may be SEQ ID NO: 3351. The αCDR3 may be SEQ ID NO: 3057 and the βCDR3 may be SEQ ID NO: 3352. The αCDR3 may be SEQ ID NO: 3064 and the βCDR3 may be SEQ ID NO: 3363. The αCDR3 may be SEQ ID NO: 3065 and the βCDR3 may be SEQ ID NO: 3364. The αCDR3 may be SEQ ID NO: 3054 and the βCDR3 may be SEQ ID NO: 3352. The αCDR3 may be SEQ ID NO: 3066 and the βCDR3 may be SEQ ID NO: 3365. The αCDR3 may be SEQ ID NO: 3067 and the βCDR3 may be SEQ ID NO: 3366. The αCDR3 may be SEQ ID NO: 3068 and the βCDR3 may be SEQ ID NO: 3367. The βCDR3 may be SEQ ID NO: 3069 and the βCDR3 may be SEQ ID NO: 3368. The αCDR3 may be SEQ ID NO: 3052 and the βCDR3 may be SEQ ID NO: 3356. The αCDR3 may be SEQ ID NO: 3070 and the βCDR3 may be SEQ ID NO: 3369. The αCDR3 may be SEQ ID NO: 3052 and the βCDR3 may be SEQ ID NO: 3355. The αCDR3 may be SEQ ID NO: 3071 and the βCDR3 may be SEQ ID NO: 3370. The αCDR3 may be SEQ ID NO: 3052 and the βCDR3 may be SEQ ID NO: 3353. The αCDR3 may be SEQ ID NO: 3072 and the βCDR3 may be SEQ ID NO: 3371. The αCDR3 may be SEQ ID NO: 3073 and the βCDR3 may be SEQ ID NO: 3372. The αCDR3 may be SEQ ID NO: 3057 and the βCDR3 may be SEQ ID NO: 3351. The αCDR3 may be SEQ ID NO: 3074 and the βCDR3 may be SEQ ID NO: 3373. The αCDR3 may be SEQ ID NO: 3075 and the βCDR3 may be SEQ ID NO: 3374. The αCDR3 may be SEQ ID NO: 3076 and the βCDR3 may be SEQ ID NO: 3375. The αCDR3 may be SEQ ID NO: 3077 and the βCDR3 may be SEQ ID NO: 3376. The αCDR3 may be SEQ ID NO: 3078 and the βCDR3 may be SEQ ID NO: 3377. The αCDR3 may be SEQ ID NO: 3079 and the βCDR3 may be SEQ ID NO: 3378. The αCDR3 may be SEQ ID NO: 3080 and the βCDR3 may be SEQ ID NO: 3379. The αCDR3 may be SEQ ID NO: 3081 and the βCDR3 may be SEQ ID NO: 3380. The αCDR3 may be SEQ ID NO: 3082 and the βCDR3 may be SEQ ID NO: 3381. The αCDR3 may be SEQ ID NO: 3083 and the βCDR3 may be SEQ ID NO: 3382. The αCDR3 may be SEQ ID NO: 3084 and the βCDR3 may be SEQ ID NO: 3383. The αCDR3 may be SEQ ID NO: 3085 and the βCDR3 may be SEQ ID NO: 3384. The αCDR3 may be SEQ ID NO: 3086 and the βCDR3 may be SEQ ID NO: 3385. The αCDR3 may be SEQ ID NO: 3087 and the βCDR3 may be SEQ ID NO: 3386. The αCDR3 may be SEQ ID NO: 3088 and the βCDR3 may be SEQ ID NO: 3387. The αCDR3 may be SEQ ID NO: 3089 and the βCDR3 may be SEQ ID NO: 3388. The αCDR3 may be SEQ ID NO: 3052 and the βCDR3 may be SEQ ID NO: 3389. The αCDR3 may be SEQ ID NO: 3056 and the βCDR3 may be SEQ ID NO: 3351. The αCDR3 may be SEQ ID NO: 3090 and the βCDR3 may be SEQ ID NO: 3390. The αCDR3 may be SEQ ID NO: 3091 and the βCDR3 may be SEQ ID NO: 3391. The αCDR3 may be SEQ ID NO: 3092 and the βCDR3 may be SEQ ID NO: 3392. The αCDR3 may be SEQ ID NO: 3093 and the βCDR3 may be SEQ ID NO: 3393. The αCDR3 may be SEQ ID NO: 3053 and the βCDR3 may be SEQ ID NO: 3356. The αCDR3 may be SEQ ID NO: 3094 and the βCDR3 may be SEQ ID NO: 3394. The αCDR3 may be SEQ ID NO: 3054 and the βCDR3 may be SEQ ID NO: 3363. The αCDR3 may be SEQ ID NO: 3095 and the βCDR3 may be SEQ ID NO: 3395. The αCDR3 may be SEQ ID NO: 3054 and the βCDR3 may be SEQ ID NO: 3351. The αCDR3 may be SEQ ID NO: 3096 and the βCDR3 may be SEQ ID NO: 3396. The αCDR3 may be SEQ ID NO: 3053 and the βCDR3 may be SEQ ID NO: 3355. The αCDR3 may be SEQ ID NO: 3097 and the βCDR3 may be SEQ ID NO: 3397. The αCDR3 may be SEQ ID NO: 3098 and the βCDR3 may be SEQ ID NO: 3398. The αCDR3 may be SEQ ID NO: 3099 and the βCDR3 may be SEQ ID NO: 3352. The αCDR3 may be SEQ ID NO: 3100 and the βCDR3 may be SEQ ID NO: 3399. The αCDR3 may be SEQ ID NO: 3053 and the βCDR3 may be SEQ ID NO: 3353. The αCDR3 may be SEQ ID NO: 3101 and the βCDR3 may be SEQ ID NO: 3400. The αCDR3 may be SEQ ID NO: 3102 and the βCDR3 may be SEQ ID NO: 3401. The αCDR3 may be SEQ ID NO: 3058 and the βCDR3 may be SEQ ID NO: 3352. The αCDR3 may be SEQ ID NO: 3103 and the βCDR3 may be SEQ ID NO: 3402. The αCDR3 may be SEQ ID NO: 3104 and the βCDR3 may be SEQ ID NO: 3403. The αCDR3 may be SEQ ID NO: 3105 and the βCDR3 may be SEQ ID NO: 3404. The αCDR3 may be SEQ ID NO: 3106 and the βCDR3 may be SEQ ID NO: 3405. The αCDR3 may be SEQ ID NO: 3107 and the βCDR3 may be SEQ ID NO: 3406. The αCDR3 may be SEQ ID NO: 3108 and the βCDR3 may be SEQ ID NO: 3407. The αCDR3 may be SEQ ID NO: 3109 and the βCDR3 may be SEQ ID NO: 3408. The αCDR3 may be SEQ ID NO: 3110 and the βCDR3 may be SEQ ID NO: 3409. The αCDR3 may be SEQ ID NO: 3111 and the βCDR3 may be SEQ ID NO: 3410. The αCDR3 may be SEQ ID NO: 3112 and the βCDR3 may be SEQ ID NO: 3411. The αCDR3 may be SEQ ID NO: 3113 and the βCDR3 may be SEQ ID NO: 3412. The αCDR3 may be SEQ ID NO: 3058 and the βCDR3 may be SEQ ID NO: 3351. The αCDR3 may be SEQ ID NO: 3052 and the βCDR3 may be SEQ ID NO: 3354. The αCDR3 may be SEQ ID NO: 3072 and the βCDR3 may be SEQ ID NO: 3353. The αCDR3 may be SEQ ID NO: 3052 and the βCDR3 may be SEQ ID NO: 3413. The αCDR3 may be SEQ ID NO: 3114 and the βCDR3 may be SEQ ID NO: 3414. The αCDR3 may be SEQ ID NO: 3058 and the βCDR3 may be SEQ ID NO: 3355. The αCDR3 may be SEQ ID NO: 3052 and the βCDR3 may be SEQ ID NO: 3415. The αCDR3 may be SEQ ID NO: 3114 and the βCDR3 may be SEQ ID NO: 3353. The αCDR3 may be SEQ ID NO: 3115 and the βCDR3 may be SEQ ID NO: 3416. The αCDR3 may be SEQ ID NO: 3116 and the βCDR3 may be SEQ ID NO: 3417. The αCDR3 may be SEQ ID NO: 3117 and the βCDR3 may be SEQ ID NO: 3418. The αCDR3 may be SEQ ID NO: 3118 and the βCDR3 may be SEQ ID NO: 3419. The αCDR3 may be SEQ ID NO: 3119 and the βCDR3 may be SEQ ID NO: 3420. The αCDR3 may be SEQ ID NO: 3120 and the βCDR3 may be SEQ ID NO: 3352. The αCDR3 may be SEQ ID NO: 3121 and the βCDR3 may be SEQ ID NO: 3421. The αCDR3 may be SEQ ID NO: 3054 and the βCDR3 may be SEQ ID NO: 3367. The αCDR3 may be SEQ ID NO: 3122 and the βCDR3 may be SEQ ID NO: 3422. The αCDR3 may be SEQ ID NO: 3123 and the βCDR3 may be SEQ ID NO: 3423. The αCDR3 may be SEQ ID NO: 3124 and the βCDR3 may be SEQ ID NO: 3424. The αCDR3 may be SEQ ID NO: 3112 and the βCDR3 may be SEQ ID NO: 3351. The αCDR3 may be SEQ ID NO: 3060 and the βCDR3 may be SEQ ID NO: 3352. The αCDR3 may be SEQ ID NO: 3059 and the βCDR3 may be SEQ ID NO: 3351. The αCDR3 may be SEQ ID NO: 3071 and the βCDR3 may be SEQ ID NO: 3355. The αCDR3 may be SEQ ID NO: 3125 and the βCDR3 may be SEQ ID NO: 3425. The αCDR3 may be SEQ ID NO: 3126 and the βCDR3 may be SEQ ID NO: 3426. The αCDR3 may be SEQ ID NO: 3127 and the βCDR3 may be SEQ ID NO: 3427. The αCDR3 may be SEQ ID NO: 3128 and the βCDR3 may be SEQ ID NO: 3428. The αCDR3 may be SEQ ID NO: 3129 and the βCDR3 may be SEQ ID NO: 3429. The αCDR3 may be SEQ ID NO: 3130 and the βCDR3 may be SEQ ID NO: 3352. The αCDR3 may be SEQ ID NO: 3052 and the βCDR3 may be SEQ ID NO: 3362. The αCDR3 may be SEQ ID NO: 3055 and the βCDR3 may be SEQ ID NO: 3352. The αCDR3 may be SEQ ID NO: 3131 and the βCDR3 may be SEQ ID NO: 3430. The αCDR3 may be SEQ ID NO: 3132 and the βCDR3 may be SEQ ID NO: 3431. The αCDR3 may be SEQ ID NO: 3133 and the βCDR3 may be SEQ ID NO: 3432. The αCDR3 may be SEQ ID NO: 3053 and the βCDR3 may be SEQ ID NO: 3381. The αCDR3 may be SEQ ID NO: 3134 and the βCDR3 may be SEQ ID NO: 3433. The αCDR3 may be SEQ ID NO: 3061 and the βCDR3 may be SEQ ID NO: 3351. The αCDR3 may be SEQ ID NO: 3104 and the βCDR3 may be SEQ ID NO: 3352. The αCDR3 may be SEQ ID NO: 3055 and the βCDR3 may be SEQ ID NO: 3351. The αCDR3 may be SEQ ID NO: 3058 and the βCDR3 may be SEQ ID NO: 3353. The αCDR3 may be SEQ ID NO: 3135 and the βCDR3 may be SEQ ID NO: 3434. The αCDR3 may be SEQ ID NO: 3052 and the βCDR3 may be SEQ ID NO: 3435. The αCDR3 may be SEQ ID NO: 3136 and the βCDR3 may be SEQ ID NO: 3436. The αCDR3 may be SEQ ID NO: 3137 and the βCDR3 may be SEQ ID NO: 3437. The αCDR3 may be SEQ ID NO: 3138 and the βCDR3 may be SEQ ID NO: 3438. The αCDR3 may be SEQ ID NO: 3139 and the βCDR3 may be SEQ ID NO: 3439. The αCDR3 may be SEQ ID NO: 3140 and the βCDR3 may be SEQ ID NO: 3440. The αCDR3 may be SEQ ID NO: 3141 and the βCDR3 may be SEQ ID NO: 3441. The αCDR3 may be SEQ ID NO: 3142 and the βCDR3 may be SEQ ID NO: 3442. The αCDR3 may be SEQ ID NO: 3143 and the βCDR3 may be SEQ ID NO: 3443. The αCDR3 may be SEQ ID NO: 3144 and the βCDR3 may be SEQ ID NO: 3444. The αCDR3 may be SEQ ID NO: 3145 and the βCDR3 may be SEQ ID NO: 3445. The αCDR3 may be SEQ ID NO: 3136 and the βCDR3 may be SEQ ID NO: 3444. The αCDR3 may be SEQ ID NO: 3146 and the βCDR3 may be SEQ ID NO: 3446. The αCDR3 may be SEQ ID NO: 3147 and the βCDR3 may be SEQ ID NO: 3447. The αCDR3 may be SEQ ID NO: 3148 and the βCDR3 may be SEQ ID NO: 3448. The αCDR3 may be SEQ ID NO: 3149 and the βCDR3 may be SEQ ID NO: 3449. The αCDR3 may be SEQ ID NO: 3150 and the βCDR3 may be SEQ ID NO: 3450. The αCDR3 may be SEQ ID NO: 3151 and the βCDR3 may be SEQ ID NO: 3436. The αCDR3 may be SEQ ID NO: 3139 and the βCDR3 may be SEQ ID NO: 3436. The αCDR3 may be SEQ ID NO: 3152 and the βCDR3 may be SEQ ID NO: 3451. The αCDR3 may be SEQ ID NO: 3153 and the βCDR3 may be SEQ ID NO: 3452. The αCDR3 may be SEQ ID NO: 3154 and the βCDR3 may be SEQ ID NO: 3453. The αCDR3 may be SEQ ID NO: 3155 and the βCDR3 may be SEQ ID NO: 3454. The αCDR3 may be SEQ ID NO: 3137 and the βCDR3 may be SEQ ID NO: 3440. The αCDR3 may be SEQ ID NO: 3156 and the βCDR3 may be SEQ ID NO: 3455. The αCDR3 may be SEQ ID NO: 3151 and the βCDR3 may be SEQ ID NO: 3456. The αCDR3 may be SEQ ID NO: 3157 and the βCDR3 may be SEQ ID NO: 3457. The αCDR3 may be SEQ ID NO: 3158 and the βCDR3 may be SEQ ID NO: 3458. The αCDR3 may be SEQ ID NO: 3159 and the βCDR3 may be SEQ ID NO: 3459. The αCDR3 may be SEQ ID NO: 3160 and the βCDR3 may be SEQ ID NO: 3460. The αCDR3 may be SEQ ID NO: 3077 and the βCDR3 may be SEQ ID NO: 3461. The αCDR3 may be SEQ ID NO: 3161 and the βCDR3 may be SEQ ID NO: 3462. The αCDR3 may be SEQ ID NO: 3162 and the βCDR3 may be SEQ ID NO: 3463. The αCDR3 may be SEQ ID NO: 3163 and the βCDR3 may be SEQ ID NO: 3464. The αCDR3 may be SEQ ID NO: 3164 and the βCDR3 may be SEQ ID NO: 3465. The αCDR3 may be SEQ ID NO: 3137 and the βCDR3 may be SEQ ID NO: 3442. The αCDR3 may be SEQ ID NO: 3136 and the βCDR3 may be SEQ ID NO: 3438. The αCDR3 may be SEQ ID NO: 3165 and the βCDR3 may be SEQ ID NO: 3466. The αCDR3 may be SEQ ID NO: 3166 and the βCDR3 may be SEQ ID NO: 3467. The αCDR3 may be SEQ ID NO: 3167 and the βCDR3 may be SEQ ID NO: 3468. The αCDR3 may be SEQ ID NO: 3168 and the βCDR3 may be SEQ ID NO: 3469. The αCDR3 may be SEQ ID NO: 3169 and the βCDR3 may be SEQ ID NO: 3470. The αCDR3 may be SEQ ID NO: 3137 and the βCDR3 may be SEQ ID NO: 3436. The αCDR3 may be SEQ ID NO: 3170 and the βCDR3 may be SEQ ID NO: 3471. The αCDR3 may be SEQ ID NO: 3171 and the βCDR3 may be SEQ ID NO: 3472. The αCDR3 may be SEQ ID NO: 3172 and the βCDR3 may be SEQ ID NO: 3473. The αCDR3 may be SEQ ID NO: 3173 and the βCDR3 may be SEQ ID NO: 3474. The αCDR3 may be SEQ ID NO: 3174 and the βCDR3 may be SEQ ID NO: 3475. The αCDR3 may be SEQ ID NO: 3175 and the βCDR3 may be SEQ ID NO: 3476. The αCDR3 may be SEQ ID NO: 3176 and the βCDR3 may be SEQ ID NO: 3477. The αCDR3 may be SEQ ID NO: 3177 and the βCDR3 may be SEQ ID NO: 3478. The αCDR3 may be SEQ ID NO: 3178 and the βCDR3 may be SEQ ID NO: 3479. The αCDR3 may be SEQ ID NO: 3179 and the βCDR3 may be SEQ ID NO: 3480. The αCDR3 may be SEQ ID NO: 3180 and the βCDR3 may be SEQ ID NO: 3481. The αCDR3 may be SEQ ID NO: 3136 and the βCDR3 may be SEQ ID NO: 3482. The αCDR3 may be SEQ ID NO: 3181 and the βCDR3 may be SEQ ID NO: 3483. The αCDR3 may be SEQ ID NO: 3182 and the βCDR3 may be SEQ ID NO: 3484. The αCDR3 may be SEQ ID NO: 3183 and the βCDR3 may be SEQ ID NO: 3485. The αCDR3 may be SEQ ID NO: 3184 and the βCDR3 may be SEQ ID NO: 3486. The αCDR3 may be SEQ ID NO: 3185 and the βCDR3 may be SEQ ID NO: 3487. The αCDR3 may be SEQ ID NO: 3186 and the βCDR3 may be SEQ ID NO: 3488. The αCDR3 may be SEQ ID NO: 3187 and the βCDR3 may be SEQ ID NO: 3489. The αCDR3 may be SEQ ID NO: 3188 and the βCDR3 may be SEQ ID NO: 3482. The αCDR3 may be SEQ ID NO: 3189 and the βCDR3 may be SEQ ID NO: 3490. The αCDR3 may be SEQ ID NO: 3190 and the βCDR3 may be SEQ ID NO: 3491. The αCDR3 may be SEQ ID NO: 3191 and the βCDR3 may be SEQ ID NO: 3492. The αCDR3 may be SEQ ID NO: 3192 and the βCDR3 may be SEQ ID NO: 3493. The αCDR3 may be SEQ ID NO: 3193 and the βCDR3 may be SEQ ID NO: 3494. The αCDR3 may be SEQ ID NO: 3194 and the βCDR3 may be SEQ ID NO: 3495. The αCDR3 may be SEQ ID NO: 3195 and the βCDR3 may be SEQ ID NO: 3496. The αCDR3 may be SEQ ID NO: 3196 and the βCDR3 may be SEQ ID NO: 3497. The αCDR3 may be SEQ ID NO: 3197 and the βCDR3 may be SEQ ID NO: 3498. The αCDR3 may be SEQ ID NO: 3198 and the βCDR3 may be SEQ ID NO: 3499. The αCDR3 may be SEQ ID NO: 3199 and the βCDR3 may be SEQ ID NO: 3500. The αCDR3 may be SEQ ID NO: 3137 and the βCDR3 may be SEQ ID NO: 3449. The αCDR3 may be SEQ ID NO: 3200 and the βCDR3 may be SEQ ID NO: 3436. The αCDR3 may be SEQ ID NO: 3201 and the βCDR3 may be SEQ ID NO: 3501. The αCDR3 may be SEQ ID NO: 3138 and the βCDR3 may be SEQ ID NO: 3436. The αCDR3 may be SEQ ID NO: 3202 and the βCDR3 may be SEQ ID NO: 3502. The αCDR3 may be SEQ ID NO: 3203 and the βCDR3 may be SEQ ID NO: 3503. The αCDR3 may be SEQ ID NO: 3204 and the βCDR3 may be SEQ ID NO: 3504. The αCDR3 may be SEQ ID NO: 3205 and the βCDR3 may be SEQ ID NO: 3505. The αCDR3 may be SEQ ID NO: 3206 and the βCDR3 may be SEQ ID NO: 3506. The αCDR3 may be SEQ ID NO: 3207 and the βCDR3 may be SEQ ID NO: 3507. The αCDR3 may be SEQ ID NO: 3148 and the βCDR3 may be SEQ ID NO: 3440. The αCDR3 may be SEQ ID NO: 3208 and the βCDR3 may be SEQ ID NO: 3508. The αCDR3 may be SEQ ID NO: 3209 and the βCDR3 may be SEQ ID NO: 3509. The αCDR3 may be SEQ ID NO: 3210 and the βCDR3 may be SEQ ID NO: 3510. The αCDR3 may be SEQ ID NO: 3211 and the βCDR3 may be SEQ ID NO: 3511. The αCDR3 may be SEQ ID NO: 3212 and the βCDR3 may be SEQ ID NO: 3512. The αCDR3 may be SEQ ID NO: 3213 and the βCDR3 may be SEQ ID NO: 3513. The αCDR3 may be SEQ ID NO: 3214 and the βCDR3 may be SEQ ID NO: 3514. The αCDR3 may be SEQ ID NO: 3215 and the βCDR3 may be SEQ ID NO: 3515. The αCDR3 may be SEQ ID NO: 3216 and the βCDR3 may be SEQ ID NO: 3516. The αCDR3 may be SEQ ID NO: 3217 and the βCDR3 may be SEQ ID NO: 3517. The αCDR3 may be SEQ ID NO: 3218 and the βCDR3 may be SEQ ID NO: 3518. The αCDR3 may be SEQ ID NO: 3219 and the βCDR3 may be SEQ ID NO: 3519. The αCDR3 may be SEQ ID NO: 3220 and the βCDR3 may be SEQ ID NO: 3520. The αCDR3 may be SEQ ID NO: 3221 and the βCDR3 may be SEQ ID NO: 3521. The αCDR3 may be SEQ ID NO: 3217 and the βCDR3 may be SEQ ID NO: 3518. The αCDR3 may be SEQ ID NO: 3222 and the βCDR3 may be SEQ ID NO: 3522. The αCDR3 may be SEQ ID NO: 3223 and the βCDR3 may be SEQ ID NO: 3523. The αCDR3 may be SEQ ID NO: 3224 and the βCDR3 may be SEQ ID NO: 3524. The αCDR3 may be SEQ ID NO: 3225 and the βCDR3 may be SEQ ID NO: 3525. The αCDR3 may be SEQ ID NO: 3226 and the βCDR3 may be SEQ ID NO: 3526. The αCDR3 may be SEQ ID NO: 3227 and the βCDR3 may be SEQ ID NO: 3527. The αCDR3 may be SEQ ID NO: 3228 and the βCDR3 may be SEQ ID NO: 3528. The αCDR3 may be SEQ ID NO: 3229 and the βCDR3 may be SEQ ID NO: 3529. The αCDR3 may be SEQ ID NO: 3230 and the βCDR3 may be SEQ ID NO: 3530. The αCDR3 may be SEQ ID NO: 3217 and the βCDR3 may be SEQ ID NO: 3525. The αCDR3 may be SEQ ID NO: 3231 and the βCDR3 may be SEQ ID NO: 3531. The αCDR3 may be SEQ ID NO: 3232 and the βCDR3 may be SEQ ID NO: 3532. The αCDR3 may be SEQ ID NO: 3233 and the βCDR3 may be SEQ ID NO: 3520. The αCDR3 may be SEQ ID NO: 3217 and the βCDR3 may be SEQ ID NO: 3530. The αCDR3 may be SEQ ID NO: 3234 and the βCDR3 may be SEQ ID NO: 3533. The αCDR3 may be SEQ ID NO: 3235 and the βCDR3 may be SEQ ID NO: 3534. The αCDR3 may be SEQ ID NO: 3217 and the βCDR3 may be SEQ ID NO: 3532. The αCDR3 may be SEQ ID NO: 3236 and the βCDR3 may be SEQ ID NO: 3535. The αCDR3 may be SEQ ID NO: 3237 and the βCDR3 may be SEQ ID NO: 3536. The αCDR3 may be SEQ ID NO: 3238 and the βCDR3 may be SEQ ID NO: 3537. The αCDR3 may be SEQ ID NO: 3239 and the βCDR3 may be SEQ ID NO: 3538. The αCDR3 may be SEQ ID NO: 3240 and the βCDR3 may be SEQ ID NO: 3539. The αCDR3 may be SEQ ID NO: 3241 and the βCDR3 may be SEQ ID NO: 3540. The αCDR3 may be SEQ ID NO: 3242 and the βCDR3 may be SEQ ID NO: 3541. The αCDR3 may be SEQ ID NO: 3243 and the βCDR3 may be SEQ ID NO: 3542. The αCDR3 may be SEQ ID NO: 3244 and the βCDR3 may be SEQ ID NO: 3543. The αCDR3 may be SEQ ID NO: 3245 and the βCDR3 may be SEQ ID NO: 3544. The αCDR3 may be SEQ ID NO: 3246 and the βCDR3 may be SEQ ID NO: 3545. The αCDR3 may be SEQ ID NO: 3247 and the βCDR3 may be SEQ ID NO: 3546. The αCDR3 may be SEQ ID NO: 3248 and the βCDR3 may be SEQ ID NO: 3547. The αCDR3 may be SEQ ID NO: 3249 and the βCDR3 may be SEQ ID NO: 3548. The αCDR3 may be SEQ ID NO: 3217 and the βCDR3 may be SEQ ID NO: 3524. The αCDR3 may be SEQ ID NO: 3250 and the βCDR3 may be SEQ ID NO: 3549. The αCDR3 may be SEQ ID NO: 3251 and the βCDR3 may be SEQ ID NO: 3550. The αCDR3 may be SEQ ID NO: 3252 and the βCDR3 may be SEQ ID NO: 3551. The αCDR3 may be SEQ ID NO: 3253 and the βCDR3 may be SEQ ID NO: 3552. The αCDR3 may be SEQ ID NO: 3254 and the βCDR3 may be SEQ ID NO: 3553. The αCDR3 may be SEQ ID NO: 3255 and the βCDR3 may be SEQ ID NO: 3554. The αCDR3 may be SEQ ID NO: 3256 and the βCDR3 may be SEQ ID NO: 3555. The αCDR3 may be SEQ ID NO: 3257 and the βCDR3 may be SEQ ID NO: 3556. The αCDR3 may be SEQ ID NO: 3258 and the βCDR3 may be SEQ ID NO: 3557. The αCDR3 may be SEQ ID NO: 3259 and the βCDR3 may be SEQ ID NO: 3558. The αCDR3 may be SEQ ID NO: 3260 and the βCDR3 may be SEQ ID NO: 3559. The αCDR3 may be SEQ ID NO: 3261 and the βCDR3 may be SEQ ID NO: 3560. The αCDR3 may be SEQ ID NO: 3217 and the βCDR3 may be SEQ ID NO: 3519. The αCDR3 may be SEQ ID NO: 3262 and the βCDR3 may be SEQ ID NO: 3561. The αCDR3 may be SEQ ID NO: 3263 and the βCDR3 may be SEQ ID NO: 3562. The αCDR3 may be SEQ ID NO: 3217 and the βCDR3 may be SEQ ID NO: 3563. The αCDR3 may be SEQ ID NO: 3264 and the βCDR3 may be SEQ ID NO: 3564. The αCDR3 may be SEQ ID NO: 3265 and the βCDR3 may be SEQ ID NO: 3565. The αCDR3 may be SEQ ID NO: 3266 and the βCDR3 may be SEQ ID NO: 3566. The αCDR3 may be SEQ ID NO: 3267 and the βCDR3 may be SEQ ID NO: 3567. The αCDR3 may be SEQ ID NO: 3268 and the βCDR3 may be SEQ ID NO: 3568. The αCDR3 may be SEQ ID NO: 3269 and the βCDR3 may be SEQ ID NO: 3569. The αCDR3 may be SEQ ID NO: 3217 and the βCDR3 may be SEQ ID NO: 3528. The αCDR3 may be SEQ ID NO: 3270 and the βCDR3 may be SEQ ID NO: 3570. The αCDR3 may be SEQ ID NO: 3217 and the βCDR3 may be SEQ ID NO: 3571. The αCDR3 may be SEQ ID NO: 3271 and the βCDR3 may be SEQ ID NO: 3572. The αCDR3 may be SEQ ID NO: 3219 and the βCDR3 may be SEQ ID NO: 3522. The αCDR3 may be SEQ ID NO: 3272 and the βCDR3 may be SEQ ID NO: 3573. The αCDR3 may be SEQ ID NO: 3273 and the βCDR3 may be SEQ ID NO: 3574. The αCDR3 may be SEQ ID NO: 3274 and the βCDR3 may be SEQ ID NO: 3575. The αCDR3 may be SEQ ID NO: 3275 and the βCDR3 may be SEQ ID NO: 3576. The αCDR3 may be SEQ ID NO: 3217 and the βCDR3 may be SEQ ID NO: 3577. The αCDR3 may be SEQ ID NO: 3230 and the βCDR3 may be SEQ ID NO: 3517. The αCDR3 may be SEQ ID NO: 3276 and the βCDR3 may be SEQ ID NO: 3578. The αCDR3 may be SEQ ID NO: 3277 and the βCDR3 may be SEQ ID NO: 3579. The αCDR3 may be SEQ ID NO: 3278 and the βCDR3 may be SEQ ID NO: 3580. The αCDR3 may be SEQ ID NO: 3279 and the βCDR3 may be SEQ ID NO: 3581. The αCDR3 may be SEQ ID NO: 3280 and the βCDR3 may be SEQ ID NO: 3582. The αCDR3 may be SEQ ID NO: 3281 and the βCDR3 may be SEQ ID NO: 3583. The αCDR3 may be SEQ ID NO: 3282 and the βCDR3 may be SEQ ID NO: 3584. The αCDR3 may be SEQ ID NO: 3283 and the βCDR3 may be SEQ ID NO: 3585. The αCDR3 may be SEQ ID NO: 3284 and the βCDR3 may be SEQ ID NO: 3586. The αCDR3 may be SEQ ID NO: 3285 and the βCDR3 may be SEQ ID NO: 3587. The αCDR3 may be SEQ ID NO: 3286 and the βCDR3 may be SEQ ID NO: 3588. The αCDR3 may be SEQ ID NO: 3287 and the βCDR3 may be SEQ ID NO: 3589. The αCDR3 may be SEQ ID NO: 3288 and the βCDR3 may be SEQ ID NO: 3590. The αCDR3 may be SEQ ID NO: 3289 and the βCDR3 may be SEQ ID NO: 3591. The αCDR3 may be SEQ ID NO: 3290 and the βCDR3 may be SEQ ID NO: 3592. The αCDR3 may be SEQ ID NO: 3291 and the βCDR3 may be SEQ ID NO: 3593. The αCDR3 may be SEQ ID NO: 3292 and the βCDR3 may be SEQ ID NO: 3594. The αCDR3 may be SEQ ID NO: 3293 and the βCDR3 may be SEQ ID NO: 3595. The αCDR3 may be SEQ ID NO: 3294 and the βCDR3 may be SEQ ID NO: 3596. The αCDR3 may be SEQ ID NO: 3295 and the βCDR3 may be SEQ ID NO: 3597. The αCDR3 may be SEQ ID NO: 3219 and the βCDR3 may be SEQ ID NO: 3598. The αCDR3 may be SEQ ID NO: 3296 and the βCDR3 may be SEQ ID NO: 3599. The αCDR3 may be SEQ ID NO: 3217 and the βCDR3 may be SEQ ID NO: 3600. The αCDR3 may be SEQ ID NO: 3297 and the βCDR3 may be SEQ ID NO: 3601. The αCDR3 may be SEQ ID NO: 3298 and the βCDR3 may be SEQ ID NO: 3602. The αCDR3 may be SEQ ID NO: 3299 and the βCDR3 may be SEQ ID NO: 3603. The αCDR3 may be SEQ ID NO: 3300 and the βCDR3 may be SEQ ID NO: 3604. The αCDR3 may be SEQ ID NO: 3301 and the βCDR3 may be SEQ ID NO: 3605. The αCDR3 may be SEQ ID NO: 3302 and the βCDR3 may be SEQ ID NO: 3606. The αCDR3 may be SEQ ID NO: 3303 and the βCDR3 may be SEQ ID NO: 3607. The αCDR3 may be SEQ ID NO: 3304 and the βCDR3 may be SEQ ID NO: 3608. The αCDR3 may be SEQ ID NO: 3305 and the βCDR3 may be SEQ ID NO: 3609. The αCDR3 may be SEQ ID NO: 3306 and the βCDR3 may be SEQ ID NO: 3610. The αCDR3 may be SEQ ID NO: 3307 and the βCDR3 may be SEQ ID NO: 3611. The αCDR3 may be SEQ ID NO: 3289 and the βCDR3 may be SEQ ID NO: 3595. The αCDR3 may be SEQ ID NO: 3308 and the βCDR3 may be SEQ ID NO: 3612. The αCDR3 may be SEQ ID NO: 3309 and the βCDR3 may be SEQ ID NO: 3613. The αCDR3 may be SEQ ID NO: 3310 and the βCDR3 may be SEQ ID NO: 3614. The αCDR3 may be SEQ ID NO: 3311 and the βCDR3 may be SEQ ID NO: 3615. The αCDR3 may be SEQ ID NO: 3312 and the βCDR3 may be SEQ ID NO: 3616. The αCDR3 may be SEQ ID NO: 3313 and the βCDR3 may be SEQ ID NO: 3617. The αCDR3 may be SEQ ID NO: 3314 and the βCDR3 may be SEQ ID NO: 3618. The αCDR3 may be SEQ ID NO: 3289 and the βCDR3 may be SEQ ID NO: 3619. The αCDR3 may be SEQ ID NO: 3315 and the βCDR3 may be SEQ ID NO: 3620. The αCDR3 may be SEQ ID NO: 3316 and the βCDR3 may be SEQ ID NO: 3621. The αCDR3 may be SEQ ID NO: 3317 and the βCDR3 may be SEQ ID NO: 3622. The αCDR3 may be SEQ ID NO: 3318 and the βCDR3 may be SEQ ID NO: 3623. The αCDR3 may be SEQ ID NO: 3319 and the βCDR3 may be SEQ ID NO: 3624. The αCDR3 may be SEQ ID NO: 3320 and the βCDR3 may be SEQ ID NO: 3625. The αCDR3 may be SEQ ID NO: 3321 and the βCDR3 may be SEQ ID NO: 3626. The αCDR3 may be SEQ ID NO: 3322 and the βCDR3 may be SEQ ID NO: 3627. The αCDR3 may be SEQ ID NO: 3323 and the βCDR3 may be SEQ ID NO: 3628. The αCDR3 may be SEQ ID NO: 3324 and the βCDR3 may be SEQ ID NO: 3629. The αCDR3 may be SEQ ID NO: 3325 and the βCDR3 may be SEQ ID NO: 3602. The αCDR3 may be SEQ ID NO: 3326 and the βCDR3 may be SEQ ID NO: 3630. The αCDR3 may be SEQ ID NO: 3327 and the βCDR3 may be SEQ ID NO: 3631. The αCDR3 may be SEQ ID NO: 3328 and the βCDR3 may be SEQ ID NO: 3632. The αCDR3 may be SEQ ID NO: 3289 and the βCDR3 may be SEQ ID NO: 3598. The αCDR3 may be SEQ ID NO: 3329 and the βCDR3 may be SEQ ID NO: 3633. The αCDR3 may be SEQ ID NO: 3330 and the βCDR3 may be SEQ ID NO: 3634. The αCDR3 may be SEQ ID NO: 3331 and the βCDR3 may be SEQ ID NO: 3635. The αCDR3 may be SEQ ID NO: 3332 and the βCDR3 may be SEQ ID NO: 3636. The αCDR3 may be SEQ ID NO: 3333 and the βCDR3 may be SEQ ID NO: 3637. The αCDR3 may be SEQ ID NO: 3334 and the βCDR3 may be SEQ ID NO: 3638. The αCDR3 may be SEQ ID NO: 3335 and the βCDR3 may be SEQ ID NO: 3639. The αCDR3 may be SEQ ID NO: 3336 and the βCDR3 may be SEQ ID NO: 3640. The αCDR3 may be SEQ ID NO: 3337 and the βCDR3 may be SEQ ID NO: 3641. The αCDR3 may be SEQ ID NO: 3338 and the βCDR3 may be SEQ ID NO: 3642. The αCDR3 may be SEQ ID NO: 3290 and the βCDR3 may be SEQ ID NO: 3596. The αCDR3 may be SEQ ID NO: 3339 and the βCDR3 may be SEQ ID NO: 3643. The αCDR3 may be SEQ ID NO: 3290 and the βCDR3 may be SEQ ID NO: 3601. The αCDR3 may be SEQ ID NO: 3340 and the βCDR3 may be SEQ ID NO: 3644. The αCDR3 may be SEQ ID NO: 3289 and the βCDR3 may be SEQ ID NO: 3611. The αCDR3 may be SEQ ID NO: 3341 and the βCDR3 may be SEQ ID NO: 3645. The αCDR3 may be SEQ ID NO: 3342 and the βCDR3 may be SEQ ID NO: 3646. The αCDR3 may be SEQ ID NO: 3343 and the βCDR3 may be SEQ ID NO: 3647. The αCDR3 may be SEQ ID NO: 3142 and the βCDR3 may be SEQ ID NO: 3648. The αCDR3 may be SEQ ID NO: 3344 and the βCDR3 may be SEQ ID NO: 3649. The αCDR3 may be SEQ ID NO: 3345 and the βCDR3 may be SEQ ID NO: 3650. The αCDR3 may be SEQ ID NO: 3290 and the βCDR3 may be SEQ ID NO: 3614. The αCDR3 may be SEQ ID NO: 3346 and the βCDR3 may be SEQ ID NO: 3651. The αCDR3 may be SEQ ID NO: 3347 and the βCDR3 may be SEQ ID NO: 3652. The αCDR3 may be SEQ ID NO: 3348 and the βCDR3 may be SEQ ID NO: 3653. The αCDR3 may be SEQ ID NO: 3349 and the βCDR3 may be SEQ ID NO: 3654. The αCDR3 may be SEQ ID NO: 3350 and the βCDR3 may be SEQ ID NO: 3655.

The TCR specific for A*01:01_EVDPIGHLY (SEQ ID NO: 3051) may comprise a TRAV, a TRAJ, a TRBV, optionally a TRBD, and a TRBJ amino acid sequence, optionally a TRAC sequence and optionally a TRBC sequence. Such TCR may comprise TRAV24, TRAJ31, TRBV3-1, TRBD1, and TRBJ2-1. Such TCR may comprise TRAV3, TRAJ6, TRBV19, and TRBJ2-1. Such TCR may comprise TRAV21, TRAJ26, TRBV27, TRBD1, and TRBJ1-6. Such TCR may comprise TRAV20, TRAJ15, TRBV27, and TRBJ2-3. Such TCR may comprise TRAV12-3, TRAJ20, TRBV20-1, TRBD2, and TRBJ1-2. Such TCR may comprise TRAV19, TRAJ40, TRBV20-1, TRBD1, and TRBJ2-7. Such TCR may comprise TRAV21, TRAJ4, TRBV10-3, and TRBJ1-1. Such TCR may comprise TRAV12-3, TRAJ20, TRBV20-1, TRBD1, and TRBJ2-7. Such TCR may comprise TRAV1-1, TRAJ4, TRBV9, TRBD1, and TRBJ1-1. Such TCR may comprise TRAV12-1, TRAJ17, TRBV6-1, TRBD2, and TRBJ2-1. Such TCR may comprise TRAV4, TRAJ47, TRBV20-1, TRBD2, and TRBJ2-3. Such TCR may comprise TRAV21, TRAJ6, TRBV5-4, and TRBJ2-1. Such TCR may comprise TRAV12-1, TRAJ11, TRBV11-3, TRBD1, and TRBJ1-1. Such TCR may comprise TRAV21, TRAJ31, TRBV5-1, TRBD1, and TRBJ1-1. Such TCR may comprise TRAV21, TRAJ33, TRBV5-1, TRBD1, and TRBJ2-3. Such TCR may comprise TRAV34, TRAJ40, TRBV9, TRBD2, and TRBJ2-7. Such TCR may comprise TRAV29DV5, TRAJ29, TRBV7-9, TRBD1, and TRBJ2-3. Such TCR may comprise TRAV19, TRAJ40, TRBV20-1, TRBD2, and TRBJ1-2. Such TCR may comprise TRAV4, TRAJ47, TRBV20-1, TRBD1, and TRBJ2-7. Such TCR may comprise TRAV21, TRAJ54, TRBV5-1, TRBD1, and TRBJ2-1. Such TCR may comprise TRAV21, TRAJ42, TRBV7-9, TRBD1, and TRBJ2-7. Such TCR may comprise TRAV21, TRAJ4, TRBV20-1, TRBD1, and TRBJ2-7. Such TCR may comprise TRAV21, TRAJ40, TRBV29-1, and TRBJ2-2. Such TCR may comprise TRAV29DV5, TRAJ49, TRBV10-2, TRBD1, and TRBJ2-7. Such TCR may comprise TRAV21, TRAJ40, TRBV27, TRBD2, and TRBJ2-2. Such TCR may comprise TRAV21, TRAJ11, TRBV5-4, and TRBJ2-2. Such TCR may comprise TRAV12-3, TRAJ20, TRBV20-1, TRBD2, and TRBJ2-3. Such TCR may comprise TRAV26-2, TRAJ49, TRBV19, and TRBJ1-5. Such TCR may comprise TRAV12-3, TRAJ20, TRBV6-1, TRBD2, and TRBJ2-1. Such TCR may comprise TRAV17, TRAJ34, TRBV11-1, TRBD1, and TRBJ1-2. Such TCR may comprise TRAV12-3, TRAJ20, TRBV10-3, and TRBJ1-1. Such TCR may comprise TRAV21, TRAJ26, TRBV5-6, TRBD1, and TRBJ2-7. Such TCR may comprise TRAV29DV5, TRAJ4, TRBV27, TRBD1, and TRBJ1-5. Such TCR may comprise TRAV4, TRAJ47, TRBV20-1, TRBD2, and TRBJ1-2. Such TCR may comprise TRAV13-1, TRAJ49, TRBV27, TRBD1, and TRBJ2-7. Such TCR may comprise TRAV12-1, TRAJ10, TRBV25-1, TRBD1, and TRBJ2-7. Such TCR may comprise TRAV29DV5, TRAJ39, TRBV7-9, and TRBJ2-7. Such TCR may comprise TRAV21, TRAJ47, TRBV9, TRBD1, and TRBJ1-1. Such TCR may comprise TRAV39, TRAJ41, TRBV13, and TRBJ1-4. Such TCR may comprise TRAV17, TRAJ53, TRBV29-1, TRBD1, and TRBJ2-1. Such TCR may comprise TRAV26-1, TRAJ42, TRBV19, TRBD1, and TRBJ2-3. Such TCR may comprise TRAV8-6, TRAJ50, TRBV9, TRBD1, and TRBJ2-7. Such TCR may comprise TRAV19, TRAJ10, TRBV7-9, and TRBJ2-7. Such TCR may comprise TRAV8-4, TRAJ42, TRBV3-1, TRBD2, and TRBJ2-1. Such TCR may comprise TRAV12-1, TRAJ47, TRBV5-8, TRBD1, and TRBJ1-1. Such TCR may comprise TRAV29DV5, TRAJ42, TRBV10-3, and TRBJ2-7. Such TCR may comprise TRAV13-2, TRAJ20, TRBV27, TRBD2, and TRBJ1-1. Such TCR may comprise TRAV10, TRAJ9, TRBV3-1, TRBD1, and TRBJ1-3. Such TCR may comprise TRAV19, TRAJ27, TRBV27, TRBD1, and TRBJ2-7. Such TCR may comprise TRAV9-2, TRAJ20, TRBV12-4, TRBD1, and TRBJ2-1. Such TCR may comprise TRAV12-2, TRAJ20, TRBV7-6, TRBD2, and TRBJ2-1. Such TCR may comprise TRAV12-1, TRAJ17, TRBV20-1, TRBD2, and TRBJ1-2. Such TCR may comprise TRAV30, TRAJ58, TRBV19, and TRBJ2-7. Such TCR may comprise TRAV8-1, TRAJ43, TRBV7-8, TRBD2, and TRBJ2-1. Such TCR may comprise TRAV13-1, TRAJ9, TRBV9, TRBD1, and TRBJ2-5. Such TCR may comprise TRAV12-1, TRAJ29, TRBV6-1, TRBD1, and TRBJ1-2. Such TCR may comprise TRAV19, TRAJ40, TRBV20-1, TRBD2, and TRBJ2-3. Such TCR may comprise TRAV21, TRAJ43, TRBV7-3, and TRBJ2-2. Such TCR may comprise TRAV21, TRAJ4, TRBV5-1, TRBD1, and TRBJ2-1. Such TCR may comprise TRAV26-2, TRAJ32, TRBV24-1, TRBD1, and TRBJ2-2. Such TCR may comprise TRAV21, TRAJ4, TRBV20-1, TRBD2, and TRBJ1-2. Such TCR may comprise TRAV19, TRAJ15, TRBV7-8, TRBD1, and TRBJ2-7. Such TCR may comprise TRAV19, TRAJ40, TRBV6-1, TRBD2, and TRBJ2-1. Such TCR may comprise TRAV12-2, TRAJ13, TRBV25-1, and TRBJ2-7. Such TCR may comprise TRAV29DV5, TRAJ54, TRBV7-8, and TRBJ2-1. Such TCR may comprise TRAV19, TRAJ53, TRBV20-1, TRBD1, and TRBJ2-7. Such TCR may comprise TRAV23DV6, TRAJ36, TRBV9, TRBD2, and TRBJ1-2. Such TCR may comprise TRAV19, TRAJ40, TRBV10-3, and TRBJ1-1. Such TCR may comprise TRAV8-6, TRAJ32, TRBV19, TRBD1, and TRBJ1-1. Such TCR may comprise TRAV1-1, TRAJ13, TRBV14, TRBD1, and TRBJ2-1. Such TCR may comprise TRAV21, TRAJ6, TRBV20-1, TRBD1, and TRBJ2-7. Such TCR may comprise TRAV21, TRAJ44, TRBV9, and TRBJ2-7. Such TCR may comprise TRAV29DV5, TRAJ3, TRBV3-1, TRBD2, and TRBJ2-5. Such TCR may comprise TRAV17, TRAJ39, TRBV7-2, and TRBJ1-2. Such TCR may comprise TRAV26-2, TRAJ12, TRBV7-9, TRBD1, and TRBJ1-2. Such TCR may comprise TRAV29DV5, TRAJ22, TRBV11-3, TRBD1, and TRBJ2-7. Such TCR may comprise TRAV21, TRAJ20, TRBV12-4, TRBD2, and TRBJ2-3. Such TCR may comprise TRAV12-3, TRAJ3, TRBV27, TRBD1, and TRBJ2-7. Such TCR may comprise TRAV27, TRAJ33, TRBV6-5, TRBD2, and TRBJ2-2. Such TCR may comprise TRAV13-1, TRAJ22, TRBV12-4, TRBD1, and TRBJ2-3. Such TCR may comprise TRAV26-1, TRAJ34, TRBV27, and TRBJ1-2. Such TCR may comprise TRAV10, TRAJ4, TRBV7-9, TRBD1, and TRBJ2-4. Such TCR may comprise TRAV21, TRAJ6, TRBV20-1, TRBD2, and TRBJ1-2. Such TCR may comprise TRAV12-3, TRAJ20, TRBV9, TRBD1, and TRBJ1-1. Such TCR may comprise TRAV21, TRAJ26, TRBV10-3, and TRBJ1-1. Such TCR may comprise TRAV12-2, TRAJ20, TRBV18, TRBD1, and TRBJ2-7. Such TCR may comprise TRAV9-2, TRAJ23, TRBV11-3, TRBD1, and TRBJ1-1. Such TCR may comprise TRAV21, TRAJ6, TRBV6-1, TRBD2, and TRBJ2-1. Such TCR may comprise TRAV12-3, TRAJ20, TRBV7-8, TRBD1, and TRBJ2-2. Such TCR may comprise TRAV9-2, TRAJ23, TRBV10-3, and TRBJ1-1. Such TCR may comprise TRAV24, TRAJ45, TRBV5-4, TRBD1, and TRBJ1-4. Such TCR may comprise TRAV13-1, TRAJ3, TRBV27, TRBD2, and TRBJ1-1. Such TCR may comprise TRAV20, TRAJ20, TRBV7-2, TRBD1, and TRBJ2-7. Such TCR may comprise TRAV8-4, TRAJ42, TRBV9, TRBD1, and TRBJ2-1. Such TCR may comprise TRAV1-2, TRAJ31, TRBV7-9, TRBD1, and TRBJ1-5. Such TCR may comprise TRAV12-1, TRAJ13, TRBV20-1, TRBD1, and TRBJ2-7. Such TCR may comprise TRAV12-1, TRAJ4, TRBV28, TRBD2, and TRBJ2-7. Such TCR may comprise TRAV21, TRAJ4, TRBV27, TRBD2, and TRBJ2-2. Such TCR may comprise TRAV3, TRAJ9, TRBV7-9, TRBD1, and TRBJ2-7. Such TCR may comprise TRAV26-1, TRAJ42, TRBV19, and TRBJ2-2. Such TCR may comprise TRAV21, TRAJ47, TRBV19, and TRBJ1-1. Such TCR may comprise TRAV26-1, TRAJ34, TRBV20-1, TRBD2, and TRBJ1-2. Such TCR may comprise TRAV21, TRAJ31, TRBV20-1, TRBD1, and TRBJ2-7. Such TCR may comprise TRAV12-1, TRAJ11, TRBV20-1, TRBD2, and TRBJ1-2. Such TCR may comprise TRAV17, TRAJ34, TRBV6-1, TRBD2, and TRBJ2-1. Such TCR may comprise TRAV13-2, TRAJ47, TRBV19, TRBD2, and TRBJ2-1. Such TCR may comprise TRAV29DV5, TRAJ28, TRBV27, TRBD2, and TRBJ2-4. Such TCR may comprise TRAV13-2, TRAJ17, TRBV27, TRBD2, and TRBJ1-5. Such TCR may comprise TRAV38-2DV8, TRAJ57, TRBV5-4, TRBD1, and TRBJ1-2. Such TCR may comprise TRAV17, TRAJ32, TRBV7-8, TRBD2, and TRBJ2-1. Such TCR may comprise TRAV21, TRAJ39, TRBV20-1, TRBD1, and TRBJ2-7. Such TCR may comprise TRAV12-3, TRAJ20, TRBV7-9, TRBD1, and TRBJ2-3. Such TCR may comprise TRAV1-1, TRAJ4, TRBV20-1, TRBD1, and TRBJ2-7. Such TCR may comprise TRAV12-1, TRAJ9, TRBV2, TRBD1, and TRBJ2-7. Such TCR may comprise TRAV19, TRAJ32, TRBV9, TRBD1, and TRBJ1-2. Such TCR may comprise TRAV8-3, TRAJ6, TRBV9, TRBD2, and TRBJ2-1. Such TCR may comprise TRAV19, TRAJ40, TRBV7-9, and TRBJ2-7. Such TCR may comprise TRAV5, TRAJ37, TRBV5-6, TRBD2, and TRBJ1-1. Such TCR may comprise TRAV21, TRAJ33, TRBV20-1, TRBD2, and TRBJ1-2. Such TCR may comprise TRAV29DV5, TRAJ3, TRBV20-1, TRBD1, and TRBJ2-7. Such TCR may comprise TRAV1-1, TRAJ4, TRBV20-1, TRBD2, and TRBJ1-2. Such TCR may comprise TRAV21, TRAJ6, TRBV10-3, and TRBJ1-1. Such TCR may comprise TRAV19, TRAJ23, TRBV9, TRBD1, and TRBJ1-1. Such TCR may comprise TRAV12-2, TRAJ20, TRBV11-2, TRBD2, and TRBJ2-2. Such TCR may comprise TRAV1-2, TRAJ15, TRBV24-1, TRBD2, and TRBJ2-1. Such TCR may comprise TRAV21, TRAJ9, TRBV5-4, and TRBJ1-6. Such TCR may comprise TRAV8-6, TRAJ12, TRBV7-9, TRBD1, and TRBJ2-2. Such TCR may comprise TRAV21, TRAJ31, TRBV11-2, TRBD2, and TRBJ1-2. Such TCR may comprise TRAV21, TRAJ41, TRBV9, TRBD1, and TRBJ1-1. Such TCR may comprise TRAV25, TRAJ28, TRBV7-2, TRBD2, and TRBJ2-6. Such TCR may comprise TRAV21, TRAJ33, TRBV10-3, TRBD1, and TRBJ1-3. Such TCR may comprise TRAV21, TRAJ49, TRBV5-1, TRBD1, and TRBJ2-5. Such TCR may comprise TRAV1-1, TRAJ34, TRBV6-6, and TRBJ1-5. Such TCR may comprise TRAV24, TRAJ6, TRBV7-2, TRBD1, and TRBJ2-1. Such TCR may comprise TRAV1-1, TRAJ15, TRBV6-6, and TRBJ1-5. Such TCR may comprise TRAV21, TRAJ15, TRBV29-1, and TRBJ1-1. Such TCR may comprise TRAV21, TRAJ43, TRBV12-4, and TRBJ1-5. Such TCR may comprise TRAV21, TRAJ30, TRBV9, TRBD1, and TRBJ1-4. Such TCR may comprise TRAV21, TRAJ31, TRBV5-1, TRBD1, and TRBJ2-7. Such TCR may comprise TRAV26-1, TRAJ45, TRBV19, TRBD2, and TRBJ2-1. Such TCR may comprise TRAV21, TRAJ43, TRBV24-1, TRBD2, and TRBJ2-1. Such TCR may comprise TRAV21, TRAJ31, TRBV24-1, TRBD2, and TRBJ2-1. Such TCR may comprise TRAV29DV5, TRAJ28, TRBV4-1, TRBD1, and TRBJ1-4. Such TCR may comprise TRAV26-2, TRAJ44, TRBV27, and TRBJ2-1. Such TCR may comprise TRAV21, TRAJ31, TRBV9, TRBD1, and TRBJ1-5. Such TCR may comprise TRAV21, TRAJ36, TRBV9, TRBD1, and TRBJ1-2. Such TCR may comprise TRAV21, TRAJ9, TRBV9, TRBD1, and TRBJ1-1. Such TCR may comprise TRAV8-3, TRAJ15, TRBV4-1, and TRBJ2-1. Such TCR may comprise TRAV21, TRAJ43, TRBV24-1, TRBD1, and TRBJ2-3. Such TCR may comprise TRAV29DV5, TRAJ40, TRBV7-9, TRBD1, and TRBJ1-6. Such TCR may comprise TRAV30, TRAJ32, TRBV28, TRBD1, and TRBJ1-1. Such TCR may comprise TRAV38-2DV8, TRAJ26, TRBV7-9, TRBD2, and TRBJ2-5. Such TCR may comprise TRAV12-1, TRAJ6, TRBV20-1, TRBD1, and TRBJ1-3. Such TCR may comprise TRAV21, TRAJ47, TRBV5-1, and TRBJ1-1. Such TCR may comprise TRAV38-2DV8, TRAJ45, TRBV29-1, TRBD1, and TRBJ2-7. Such TCR may comprise TRAV21, TRAJ15, TRBV7-2, and TRBJ1-1. Such TCR may comprise TRAV12-2, TRAJ29, TRBV9, TRBD1, and TRBJ1-1. Such TCR may comprise TRAV3, TRAJ6, TRBV28, TRBD1, and TRBJ2-7. Such TCR may comprise TRAV21, TRAJ9, TRBV10-3, TRBD1, and TRBJ1-3. Such TCR may comprise TRAV1-2, TRAJ15, TRBV7-9, TRBD1, and TRBJ2-2. Such TCR may comprise TRAV8-6, TRAJ40, TRBV15, and TRBJ2-5. Such TCR may comprise TRAV38-2DV8, TRAJ57, TRBV13, TRBD1, and TRBJ1-4. Such TCR may comprise TRAV8-6, TRAJ10, TRBV7-9, and TRBJ1-1. Such TCR may comprise TRAV21, TRAJ20, TRBV5-4, TRBD1, and TRBJ1-5. Such TCR may comprise TRAV13-1, TRAJ28, TRBV7-8, TRBD1, and TRBJ1-5. Such TCR may comprise TRAV21, TRAJ9, TRBV24-1, TRBD2, and TRBJ2-1. Such TCR may comprise TRAV1-2, TRAJ15, TRBV2, TRBD2, and TRBJ2-1. Such TCR may comprise TRAV35, TRAJ26, TRBV27, TRBD1, and TRBJ1-1. Such TCR may comprise TRAV38-2DV8, TRAJ43, TRBV5-1, TRBD2, and TRBJ2-5. Such TCR may comprise TRAV5, TRAJ32, TRBV19, TRBD2, and TRBJ2-7. Such TCR may comprise TRAV13-1, TRAJ21, TRBV5-1, TRBD2, and TRBJ2-7. Such TCR may comprise TRAV12-2, TRAJ45, TRBV12-4, TRBD1, and TRBJ2-1. Such TCR may comprise TRAV21, TRAJ31, TRBV12-5, and TRBJ2-2. Such TCR may comprise TRAV24, TRAJ52, TRBV27, and TRBJ2-1. Such TCR may comprise TRAV21, TRAJ52, TRBV19, TRBD1, and TRBJ1-1. Such TCR may comprise TRAV36DV7, TRAJ44, TRBV7-9, TRBD1, and TRBJ2-2. Such TCR may comprise TRAV3, TRAJ29, TRBV11-2, TRBD1, and TRBJ2-5. Such TCR may comprise TRAV1-1, TRAJ15, TRBV13, TRBD1, and TRBJ1-2. Such TCR may comprise TRAV29DV5, TRAJ52, TRBV11-3, TRBD1, and TRBJ2-3. Such TCR may comprise TRAV12-1, TRAJ6, TRBV19, TRBD1, and TRBJ1-1. Such TCR may comprise TRAV19, TRAJ13, TRBV27, TRBD2, and TRBJ2-7. Such TCR may comprise TRAV17, TRAJ43, TRBV12-3, and TRBJ1-4. Such TCR may comprise TRAV12-3, TRAJ20, TRBV12-4, and TRBJ2-1. Such TCR may comprise TRAV21, TRAJ52, TRBV4-1, TRBD2, and TRBJ2-7. Such TCR may comprise TRAV21, TRAJ23, TRBV19, TRBD1, and TRBJ2-7. Such TCR may comprise TRAV1-1, TRAJ30, TRBV13, TRBD1, and TRBJ1-2. Such TCR may comprise TRAV12-2, TRAJ43, TRBV12-4, TRBD2, and TRBJ2-7. Such TCR may comprise TRAV24, TRAJ10, TRBV5-1, TRBD1, and TRBJ1-2. Such TCR may comprise TRAV5, TRAJ9, TRBV4-1, TRBD2, and TRBJ1-1. Such TCR may comprise TRAV21, TRAJ40, TRBV7-8, and TRBJ1-1. Such TCR may comprise TRAV13-1, TRAJ45, TRBV9, TRBD1, and TRBJ1-6. Such TCR may comprise TRAV12-1, TRAJ26, TRBV4-1, TRBD1, and TRBJ2-7. Such TCR may comprise TRAV26-2, TRAJ45, TRBV19, and TRBJ1-2. Such TCR may comprise TRAV22, TRAJ23, TRBV5-4, TRBD1, and TRBJ1-1. Such TCR may comprise TRAV19, TRAJ42, TRBV28, and TRBJ2-7. Such TCR may comprise TRAV17, TRAJ52, TRBV7-8, TRBD1, and TRBJ1-2. Such TCR may comprise TRAV12-1, TRAJ39, TRBV3-1, TRBD1, and TRBJ2-3. Such TCR may comprise TRAV21, TRAJ9, TRBV5-1, TRBD1, and TRBJ2-7. Such TCR may comprise TRAV1-1, TRAJ5, TRBV24-1, TRBD2, and TRBJ2-1. Such TCR may comprise TRAV23DV6, TRAJ13, TRBV6-5, TRBD1, and TRBJ2-1. Such TCR may comprise TRAV8-6, TRAJ12, TRBV24-1, TRBD2, and TRBJ2-1. Such TCR may comprise TRAV1-2, TRAJ28, TRBV27, and TRBJ2-3. Such TCR may comprise TRAV29DV5, TRAJ34, TRBV4-1, TRBD2, and TRBJ2-3. Such TCR may comprise TRAV12-1, TRAJ21, TRBV28, TRBD1, and TRBJ1-5. Such TCR may comprise TRAV9-2, TRAJ29, TRBV5-8, TRBD2, and TRBJ1-1. Such TCR may comprise TRAV27, TRAJ40, TRBV7-6, and TRBJ2-7. Such TCR may comprise TRAV21, TRAJ31, TRBV7-8, TRBD1, and TRBJ1-2. Such TCR may comprise TRAV21, TRAJ30, TRBV9, TRBD1, and TRBJ1-1. Such TCR may comprise TRAV19, TRAJ30, TRBV20-1, and TRBJ2-1. Such TCR may comprise TRAV1-1, TRAJ26, TRBV12-5, TRBD2, and TRBJ2-1. Such TCR may comprise TRAV1-2, TRAJ33, TRBV9, TRBD2, and TRBJ2-3. Such TCR may comprise TRAV26-1, TRAJ50, TRBV27, TRBD1, and TRBJ2-3. Such TCR may comprise TRAV40, TRAJ41, TRBV6-5, TRBD2, and TRBJ1-2. Such TCR may comprise TRAV12-2, TRAJ31, TRBV7-9, TRBD1, and TRBJ1-5. Such TCR may comprise TRAV5, TRAJ43, TRBV5-1, TRBD1, and TRBJ2-3. Such TCR may comprise TRAV24, TRAJ52, TRBV5-1, and TRBJ1-1. Such TCR may comprise TRAV1-2, TRAJ11, TRBV7-6, TRBD1, and TRBJ1-3. Such TCR may comprise TRAV21, TRAJ33, TRBV5-1, TRBD1, and TRBJ2-7. Such TCR may comprise TRAV21, TRAJ39, TRBV10-3, and TRBJ2-1. Such TCR may comprise TRAV21, TRAJ20, TRBV14, TRBD1, and TRBJ1-2. Such TCR may comprise TRAV29DV5, TRAJ48, TRBV7-9, TRBD1, and TRBJ1-2. Such TCR may comprise TRAV13-1, TRAJ22, TRBV29-1, and TRBJ1-1. Such TCR may comprise TRAV21, TRAJ33, TRBV10-3, and TRBJ2-1. Such TCR may comprise TRAV39, TRAJ49, TRBV24-1, TRBD1, and TRBJ1-4. Such TCR may comprise TRAV13-1, TRAJ23, TRBV27, TRBD1, and TRBJ1-2. Such TCR may comprise TRAV21, TRAJ9, TRBV9, TRBD1, and TRBJ2-7. Such TCR may comprise TRAV21, TRAJ33, TRBV9, and TRBJ1-1. Such TCR may comprise TRAV19, TRAJ28, TRBV19, TRBD1, and TRBJ1-4. Such TCR may comprise TRAV10, TRAJ8, TRBV5-1, TRBD1, and TRBJ2-7. Such TCR may comprise TRAV21, TRAJ48, TRBV27, TRBD2, and TRBJ2-2. Such TCR may comprise TRAV12-2, TRAJ4, TRBV7-2, TRBD2, and TRBJ2-1. Such TCR may comprise TRAV21, TRAJ31, TRBV5-1, and TRBJ1-1. Such TCR may comprise TRAV21, TRAJ33, TRBV9, and TRBJ1-1. Such TCR may comprise TRAV21, TRAJ6, TRBV6-6, TRBD1, and TRBJ1-5. Such TCR may comprise TRAV21, TRAJ29, TRBV5-1, TRBD2, and TRBJ2-5. Such TCR may comprise TRAV41, TRAJ41, TRBV7-9, TRBD1, and TRBJ1-2. Such TCR may comprise TRAV21, TRAJ33, TRBV5-1, and TRBJ1-1. Such TCR may comprise TRAV17, TRAJ39, TRBV27, TRBD2, and TRBJ2-1. Such TCR may comprise TRAV13-2, TRAJ13, TRBV9, and TRBJ1-3. Such TCR may comprise TRAV21, TRAJ33, TRBV5-1, TRBD2, and TRBJ2-5. Such TCR may comprise TRAV17, TRAJ57, TRBV9, TRBD2, and TRBJ1-1. Such TCR may comprise TRAV5, TRAJ44, TRBV7-9, TRBD1, and TRBJ1-1. Such TCR may comprise TRAV3, TRAJ39, TRBV27, TRBD1, and TRBJ1-5. Such TCR may comprise TRAV1-2, TRAJ4, TRBV11-1, TRBD2, and TRBJ2-1. Such TCR may comprise TRAV38-2DV8, TRAJ40, TRBV7-8, TRBD1, and TRBJ2-7. Such TCR may comprise TRAV8-3, TRAJ41, TRBV7-9, and TRBJ1-1. Such TCR may comprise TRAV5, TRAJ4, TRBV11-2, and TRBJ2-1. Such TCR may comprise TRAV24, TRAJ49, TRBV6-5, TRBD1, and TRBJ1-1. Such TCR may comprise TRAV4, TRAJ45, TRBV24-1, TRBD2, and TRBJ1-1. Such TCR may comprise TRAV29DV5, TRAJ48, TRBV20-1, TRBD2, and TRBJ2-7. Such TCR may comprise TRAV26-2, TRAJ44, TRBV6-1, and TRBJ2-7. Such TCR may comprise TRAV21, TRAJ27, TRBV7-9, and TRBJ1-6. Such TCR may comprise TRAV26-1, TRAJ49, TRBV7-9, and TRBJ2-7. Such TCR may comprise TRAV12-1, TRAJ5, TRBV7-8, TRBD1, and TRBJ2-1. Such TCR may comprise TRAV21, TRAJ33, TRBV9, TRBD1, and TRBJ2-7. Such TCR may comprise TRAV21, TRAJ20, TRBV27, TRBD1, and TRBJ2-4. Such TCR may comprise TRAV39, TRAJ42, TRBV9, TRBD1, and TRBJ2-7. Such TCR may comprise TRAV1-2, TRAJ39, TRBV27, TRBD2, and TRBJ1-4. Such TCR may comprise TRAV1-1, TRAJ34, TRBV9, TRBD1, and TRBJ2-3. Such TCR may comprise TRAV25, TRAJ34, TRBV29-1, TRBD1, and TRBJ1-2. Such TCR may comprise TRAV39, TRAJ39, TRBV30, TRBD1, and TRBJ2-1. Such TCR may comprise TRAV21, TRAJ6, TRBV20-1, TRBD2, and TRBJ1-1. Such TCR may comprise TRAV8-6, TRAJ30, TRBV9, TRBD2, and TRBJ2-2. Such TCR may comprise TRAV21, TRAJ18, TRBV27, TRBD1, and TRBJ1-2. Such TCR may comprise TRAV12-3, TRAJ23, TRBV11-3, TRBD1, and TRBJ2-2. Such TCR may comprise TRAV12-1, TRAJ47, TRBV5-6, and TRBJ1-2. Such TCR may comprise TRAV22, TRAJ31, TRBV5-6, and TRBJ2-7. Such TCR may comprise TRAV21, TRAJ33, TRBV14, TRBD1, and TRBJ1-2. Such TCR may comprise TRAV1-2, TRAJ31, TRBV2, TRBD2, and TRBJ2-7. Such TCR may comprise TRAV1-2, TRAJ5, TRBV20-1, TRBD2, and TRBJ2-7. Such TCR may comprise TRAV21, TRAJ33, TRBV5-1, TRBD1, and TRBJ1-2. Such TCR may comprise TRAV16, TRAJ28, TRBV7-9, TRBD1, and TRBJ2-1. Such TCR may comprise TRAV13-1, TRAJ12, TRBV20-1, TRBD2, and TRBJ1-1. Such TCR may comprise TRAV17, TRAJ52, TRBV29-1, TRBD2, and TRBJ2-1. Such TCR may comprise TRAV36DV7, TRAJ49, TRBV15, TRBD2, and TRBJ2-3. Such TCR may comprise TRAV12-3, TRAJ58, TRBV12-4, TRBD2, and TRBJ2-1. Such TCR may comprise TRAV16, TRAJ18, TRBV27, TRBD1, and TRBJ2-7. Such TCR may comprise TRAV21, TRAJ33, TRBV27, TRBD2, and TRBJ2-2. Such TCR may comprise TRAV12-2, TRAJ48, TRBV27, and TRBJ2-6. Such TCR may comprise TRAV21, TRAJ33, TRBV2, TRBD1, and TRBJ1-2. Such TCR may comprise TRAV29DV5, TRAJ37, TRBV5-4, TRBD2, and TRBJ2-7. Such TCR may comprise TRAV21, TRAJ20, TRBV24-1, TRBD1, and TRBJ1-4. Such TCR may comprise TRAV12-2, TRAJ6, TRBV15, TRBD1, and TRBJ2-2. Such TCR may comprise TRAV12-1, TRAJ42, TRBV27, TRBD1, and TRBJ1-5. Such TCR may comprise TRAV1-1, TRAJ23, TRBV25-1, TRBD1, and TRBJ2-7. Such TCR may comprise TRAV38-1, TRAJ28, TRBV5-1, TRBD1, and TRBJ2-1. Such TCR may comprise TRAV21, TRAJ33, TRBV2, TRBD1, and TRBJ1-1. Such TCR may comprise TRAV21, TRAJ31, TRBV5-1, TRBD1, and TRBJ2-7. Such TCR may comprise TRAV8-6, TRAJ42, TRBV27, and TRBJ1-1. Such TCR may comprise TRAV40, TRAJ32, TRBV7-6, and TRBJ2-2. Such TCR may comprise TRAV5, TRAJ5, TRBV20-1, TRBD1, and TRBJ2-5. Such TCR may comprise TRAV12-1, TRAJ40, TRBV4-1, and TRBJ2-5. Such TCR may comprise TRAV13-2, TRAJ53, TRBV5-1, and TRBJ1-1. Such TCR may comprise TRAV12-2, TRAJ48, TRBV5-6, TRBD1, and TRBJ2-2. Such TCR may comprise TRAV12-3, TRAJ15, TRBV20-1, and TRBJ2-7. Such TCR may comprise TRAV12-3, TRAJ23, TRBV13, TRBD1, and TRBJ2-3. Such TCR may comprise TRAV13-2, TRAJ9, TRBV7-3, and TRBJ1-6. Such TCR may comprise TRAV21, TRAJ45, TRBV5-1, and TRBJ1-1. Such TCR may comprise TRAV25, TRAJ31, TRBV29-1, TRBD1, and TRBJ1-2. Such TCR may comprise TRAV34, TRAJ37, TRBV28, and TRBJ1-1. Such TCR may comprise TRAV1-2, TRAJ9, TRBV9, TRBD1, and TRBJ2-6. Such TCR may comprise TRAV21, TRAJ36, TRBV9, TRBD1, and TRBJ2-7. Such TCR may comprise TRAV12-1, TRAJ34, TRBV6-1, and TRBJ2-7. Such TCR may comprise TRAV12-1, TRAJ26, TRBV11-3, TRBD1, and TRBJ1-1. Such TCR may comprise TRAV17, TRAJ36, TRBV5-4, and TRBJ2-1. Such TCR may comprise TRAV21, TRAJ49, TRBV4-1, TRBD1, and TRBJ1-1. Such TCR may comprise TRAV12-1, TRAJ13, TRBV9, TRBD2, and TRBJ2-7. Such TCR may comprise TRAV24, TRAJ7, TRBV7-9, TRBD1, and TRBJ2-1. Such TCR may comprise TRAV21, TRAJ20, TRBV9, TRBD2, and TRBJ1-1. Such TCR may comprise TRAV13-2, TRAJ49, TRBV6-1, TRBD1, and TRBJ2-5. Such TCR may comprise TRAV21, TRAJ33, TRBV5-5, TRBD1, and TRBJ1-2. Such TCR may comprise TRAV12-1, TRAJ39, TRBV4-2, TRBD2, and TRBJ2-7. Such TCR may comprise TRAV26-2, TRAJ30, TRBV9, TRBD1, and TRBJ2-7. Such TCR may comprise TRAV20, TRAJ45, TRBV5-4, TRBD1, and TRBJ2-7. Such TCR may comprise TRAV21, TRAJ31, TRBV7-8, TRBD2, and TRBJ1-2. Such TCR may comprise TRAV38-2DV8, TRAJ48, TRBV2, TRBD1, and TRBJ1-5. Such TCR may comprise TRAV25, TRAJ15, TRBV9, TRBD1, and TRBJ2-1. Such TCR may comprise TRAV21, TRAJ49, TRBV5-4, and TRBJ2-7. Such TCR may comprise TRAV21, TRAJ12, TRBV27, TRBD1, and TRBJ2-2. Such TCR may comprise TRAV38-2DV8, TRAJ54, TRBV24-1, and TRBJ2-2. Such TCR may comprise TRAV17, TRAJ52, TRBV27, and TRBJ2-1. Such TCR may comprise TRAV21, TRAJ28, TRBV9, TRBD2, and TRBJ2-7. Such TCR may comprise TRAV21, TRAJ36, TRBV4-1, TRBD1, and TRBJ1-1. Such TCR may comprise TRAV21, TRAJ31, TRBV5-4, and TRBJ1-2. Such TCR may comprise TRAV21, TRAJ33, TRBV5-1, TRBD1, and TRBJ2-3. Such TCR may comprise TRAV12-1, TRAJ43, TRBV6-5, TRBD1, and TRBJ2-7. Such TCR may comprise TRAV21, TRAJ41, TRBV9, and TRBJ2-2. Such TCR may comprise TRAV19, TRAJ40, TRBV20-1, and TRBJ2-7. Such TCR may comprise TRAV12-2, TRAJ52, TRBV6-1, TRBD2, and TRBJ2-7. Such TCR may comprise TRAV26-1, TRAJ57, TRBV2, and TRBJ2-7. Such TCR may comprise TRAV21, TRAJ36, TRBV12-4, TRBD1, and TRBJ1-6. Such TCR may comprise TRAV8-4, TRAJ34, TRBV7-9, and TRBJ2-7. Such TCR may comprise TRAV19, TRAJ32, TRBV7-9, and TRBJ1-2. Such TCR may comprise TRAV21, TRAJ6, TRBV3-1, TRBD2, and TRBJ1-4. Such TCR may comprise TRAV13-2, TRAJ29, TRBV5-1, and TRBJ2-2. Such TCR may comprise TRAV14DV4, TRAJ26, TRBV7-9, TRBD1, and TRBJ2-5. Such TCR may comprise TRAV35, TRAJ44, TRBV27, TRBD1, and TRBJ2-1. Such TCR may comprise TRAV21, TRAJ24, TRBV27, TRBD1, and TRBJ1-6. Such TCR may comprise TRAV25, TRAJ21, TRBV28, TRBD1, and TRBJ2-7. Such TCR may comprise TRAV3, TRAJ36, TRBV28, and TRBJ1-5. Such TCR may comprise TRAV26-2, TRAJ52, TRBV5-6, TRBD2, and TRBJ2-1. Such TCR may comprise TRAV8-6, TRAJ40, TRBV9, TRBD1, and TRBJ2-7. Such TCR may comprise TRAV21, TRAJ42, TRBV28, TRBD1, and TRBJ2-7. Such TCR may comprise TRAV12-1, TRAJ32, TRBV20-1, TRBD1, and TRBJ1-1. Such TCR may comprise TRAV24, TRAJ24, TRBV28, TRBD2, and TRBJ2-5. Such TCR may comprise TRAV21, TRAJ36, TRBV9, TRBD2, and TRBJ1-1. Such TCR may comprise TRAV12-1, TRAJ26, TRBV2, and TRBJ1-6. Such TCR may comprise TRAV21, TRAJ31, TRBV29-1, TRBD1, and TRBJ1-1. Such TCR may comprise TRAV39, TRAJ33, TRBV6-1, and TRBJ1-5. Such TCR may comprise TRAV3, TRAJ38, TRBV27, TRBD2, and TRBJ2-7. Such TCR may comprise TRAV10, TRAJ33, TRBV30, TRBD2, and TRBJ2-1. Such TCR may comprise TRAV21, TRAJ20, TRBV2, TRBD1, and TRBJ2-1. Such TCR may comprise TRAV13-1, TRAJ20, TRBV5-1, TRBD1, and TRBJ1-1. Such TCR may comprise TRAV27, TRAJ45, TRBV27, TRBD1, and TRBJ1-6. Such TCR may comprise TRAV21, TRAJ18, TRBV9, TRBD1, and TRBJ2-1. Such TCR may comprise TRAV26-2, TRAJ28, TRBV27, and TRBJ1-5. Such TCR may comprise TRAV12-1, TRAJ34, TRBV9, TRBD2, and TRBJ2-7. Such TCR may comprise TRAV13-2, TRAJ40, TRBV4-1, and TRBJ1-3. Such TCR may comprise TRAV12-1, TRAJ34, TRBV4-2, TRBD2, and TRBJ2-7. Such TCR may comprise TRAV13-2, TRAJ46, TRBV7-9, TRBD1, and TRBJ2-1. Such TCR may comprise TRAV21, TRAJ36, TRBV9, TRBD2, and TRBJ2-7. Such TCR may comprise TRAV1-2, TRAJ20, TRBV11-3, TRBD1, and TRBJ2-3. Such TCR may comprise TRAV3, TRAJ6, TRBV12-4, TRBD1, and TRBJ2-2. Such TCR may comprise TRAV25, TRAJ32, TRBV19, TRBD1, and TRBJ1-1. Such TCR may comprise TRAV21, TRAJ33, TRBV9, TRBD1, and TRBJ1-1. Such TCR may comprise TRAV19, TRAJ53, TRBV7-7, TRBD1, and TRBJ2-1. Such TCR may comprise TRAV12-1, TRAJ20, TRBV10-3, TRBD2, and TRBJ2-3. Such TCR may comprise TRAV12-1, TRAJ34, TRBV6-5, TRBD1, and TRBJ2-7. Such TCR may comprise TRAV26-2, TRAJ43, TRBV25-1, TRBD1, and TRBJ1-2. Such TCR may comprise TRAV8-6, TRAJ20, TRBV7-9, TRBD1, and TRBJ2-2. Such TCR may comprise TRAV3, TRAJ18, TRBV20-1, TRBD2, and TRBJ2-1. Such TCR may comprise TRAV21, TRAJ40, TRBV11-3, TRBD1, and TRBJ1-2. Such TCR may comprise TRAV2, TRAJ10, TRBV6-5, TRBD2, and TRBJ2-7.

The TCR specific for A*01:01_EVDPIGHLY (SEQ ID NO: 3051) may comprise an alpha VJ sequence. The alpha VJ sequence may be any one of SEQ ID NOS 3656-3961 or 4302-4305.

The TCR specific for A*01:01_EVDPIGHLY (SEQ ID NO: 3051) may comprise a beta V(D)J sequence. The beta V(D)J sequence may be any one of SEQ ID NOS 3962-4269 or 4317-4320.

In some embodiments, the alpha VJ sequence is SEQ ID NO: 3656 and the beta V(D)J sequence is SEQ ID NO: 3962. In some embodiments, the alpha VJ sequence is SEQ ID NO: 3657 and the beta V(D)J sequence is SEQ ID NO: 3963. In some embodiments, the alpha VJ sequence is SEQ ID NO: 3658 and the beta V(D)J sequence is SEQ ID NO: 3964. In some embodiments, the alpha VJ sequence is SEQ ID NO: 3659 and the beta V(D)J sequence is SEQ ID NO: 3963. In some embodiments, the alpha VJ sequence is SEQ ID NO: 3660 and the beta V(D)J sequence is SEQ ID NO: 3965. In some embodiments, the alpha VJ sequence is SEQ ID NO: 3661 and the beta V(D)J sequence is SEQ ID NO: 3966. In some embodiments, the alpha VJ sequence is SEQ ID NO: 3662 and the beta V(D)J sequence is SEQ ID NO: 3967. In some embodiments, the alpha VJ sequence is SEQ ID NO: 3663 and the beta V(D)J sequence is SEQ ID NO: 3968. In some embodiments, the alpha VJ sequence is SEQ ID NO: 3664 and the beta V(D)J sequence is SEQ ID NO: 3969. In some embodiments, the alpha VJ sequence is SEQ ID NO: 3665 and the beta V(D)J sequence is SEQ ID NO: 3970. In some embodiments, the alpha VJ sequence is SEQ ID NO: 3666 and the beta V(D)J sequence is SEQ ID NO: 3971. In some embodiments, the alpha VJ sequence is SEQ ID NO: 3667 and the beta V(D)J sequence is SEQ ID NO: 3972. In some embodiments, the alpha VJ sequence is SEQ ID NO: 3668 and the beta V(D)J sequence is SEQ ID NO: 3973. In some embodiments, the alpha VJ sequence is SEQ ID NO: 3657 and the beta V(D)J sequence is SEQ ID NO: 3962. In some embodiments, the alpha VJ sequence is SEQ ID NO: 3662 and the beta V(D)J sequence is SEQ ID NO: 3963. In some embodiments, the alpha VJ sequence is SEQ ID NO: 3669 and the beta V(D)J sequence is SEQ ID NO: 3974. In some embodiments, the alpha VJ sequence is SEQ ID NO: 3670 and the beta V(D)J sequence is SEQ ID NO: 3975. In some embodiments, the alpha VJ sequence is SEQ ID NO: 3658 and the beta V(D)J sequence is SEQ ID NO: 3963. In some embodiments, the alpha VJ sequence is SEQ ID NO: 3671 and the beta V(D)J sequence is SEQ ID NO: 3976. In some embodiments, the alpha VJ sequence is SEQ ID NO: 3672 and the beta V(D)J sequence is SEQ ID NO: 3977. In some embodiments, the alpha VJ sequence is SEQ ID NO: 3673 and the beta V(D)J sequence is SEQ ID NO: 3978. In some embodiments, the alpha VJ sequence is SEQ ID NO: 3674 and the beta V(D)J sequence is SEQ ID NO: 3979. In some embodiments, the alpha VJ sequence is SEQ ID NO: 3659 and the beta V(D)J sequence is SEQ ID NO: 3967. In some embodiments, the alpha VJ sequence is SEQ ID NO: 3675 and the beta V(D)J sequence is SEQ ID NO: 3980. In some embodiments, the alpha VJ sequence is SEQ ID NO: 3659 and the beta V(D)J sequence is SEQ ID NO: 3966. In some embodiments, the alpha VJ sequence is SEQ ID NO: 3676 and the beta V(D)J sequence is SEQ ID NO: 3981. In some embodiments, the alpha VJ sequence is SEQ ID NO: 3659 and the beta V(D)J sequence is SEQ ID NO: 3964. In some embodiments, the alpha VJ sequence is SEQ ID NO: 3677 and the beta V(D)J sequence is SEQ ID NO: 3982. In some embodiments, the alpha VJ sequence is SEQ ID NO: 3678 and the beta V(D)J sequence is SEQ ID NO: 3983. In some embodiments, the alpha VJ sequence is SEQ ID NO: 3662 and the beta V(D)J sequence is SEQ ID NO: 3962. In some embodiments, the alpha VJ sequence is SEQ ID NO: 3679 and the beta V(D)J sequence is SEQ ID NO: 3984. In some embodiments, the alpha VJ sequence is SEQ ID NO: 3680 and the beta V(D)J sequence is SEQ ID NO: 3985. In some embodiments, the alpha VJ sequence is SEQ ID NO: 3681 and the beta V(D)J sequence is SEQ ID NO: 3986. In some embodiments, the alpha VJ sequence is SEQ ID NO: 3682 and the beta V(D)J sequence is SEQ ID NO: 3987. In some embodiments, the alpha VJ sequence is SEQ ID NO: 3683 and the beta V(D)J sequence is SEQ ID NO: 3988. In some embodiments, the alpha VJ sequence is SEQ ID NO: 3684 and the beta V(D)J sequence is SEQ ID NO: 3989. In some embodiments, the alpha VJ sequence is SEQ ID NO: 3685 and the beta V(D)J sequence is SEQ ID NO: 3990. In some embodiments, the alpha VJ sequence is SEQ ID NO: 3686 and the beta V(D)J sequence is SEQ ID NO: 3991. In some embodiments, the alpha VJ sequence is SEQ ID NO: 3687 and the beta V(D)J sequence is SEQ ID NO: 3992. In some embodiments, the alpha VJ sequence is SEQ ID NO: 3688 and the beta V(D)J sequence is SEQ ID NO: 3993. In some embodiments, the alpha VJ sequence is SEQ ID NO: 3689 and the beta V(D)J sequence is SEQ ID NO: 3994. In some embodiments, the alpha VJ sequence is SEQ ID NO: 3690 and the beta V(D)J sequence is SEQ ID NO: 3995. In some embodiments, the alpha VJ sequence is SEQ ID NO: 3691 and the beta V(D)J sequence is SEQ ID NO: 3996. In some embodiments, the alpha VJ sequence is SEQ ID NO: 3692 and the beta V(D)J sequence is SEQ ID NO: 3997. In some embodiments, the alpha VJ sequence is SEQ ID NO: 3693 and the beta V(D)J sequence is SEQ ID NO: 3998. In some embodiments, the alpha VJ sequence is SEQ ID NO: 3694 and the beta V(D)J sequence is SEQ ID NO: 3999. In some embodiments, the alpha VJ sequence is SEQ ID NO: 3695 and the beta V(D)J sequence is SEQ ID NO: 4000. In some embodiments, the alpha VJ sequence is SEQ ID NO: 3661 and the beta V(D)J sequence is SEQ ID NO: 3962. In some embodiments, the alpha VJ sequence is SEQ ID NO: 3696 and the beta V(D)J sequence is SEQ ID NO: 4001. In some embodiments, the alpha VJ sequence is SEQ ID NO: 3697 and the beta V(D)J sequence is SEQ ID NO: 4002. In some embodiments, the alpha VJ sequence is SEQ ID NO: 3698 and the beta V(D)J sequence is SEQ ID NO: 4003. In some embodiments, the alpha VJ sequence is SEQ ID NO: 3699 and the beta V(D)J sequence is SEQ ID NO: 4004. In some embodiments, the alpha VJ sequence is SEQ ID NO: 3700 and the beta V(D)J sequence is SEQ ID NO: 3967. In some embodiments, the alpha VJ sequence is SEQ ID NO: 3701 and the beta V(D)J sequence is SEQ ID NO: 4005. In some embodiments, the alpha VJ sequence is SEQ ID NO: 3658 and the beta V(D)J sequence is SEQ ID NO: 3974. In some embodiments, the alpha VJ sequence is SEQ ID NO: 3702 and the beta V(D)J sequence is SEQ ID NO: 4006. In some embodiments, the alpha VJ sequence is SEQ ID NO: 3658 and the beta V(D)J sequence is SEQ ID NO: 3962. In some embodiments, the alpha VJ sequence is SEQ ID NO: 3703 and the beta V(D)J sequence is SEQ ID NO: 4007. In some embodiments, the alpha VJ sequence is SEQ ID NO: 3657 and the beta V(D)J sequence is SEQ ID NO: 3966. In some embodiments, the alpha VJ sequence is SEQ ID NO: 3704 and the beta V(D)J sequence is SEQ ID NO: 4008. In some embodiments, the alpha VJ sequence is SEQ ID NO: 3705 and the beta V(D)J sequence is SEQ ID NO: 4009. In some embodiments, the alpha VJ sequence is SEQ ID NO: 3706 and the beta V(D)J sequence is SEQ ID NO: 3963. In some embodiments, the alpha VJ sequence is SEQ ID NO: 3707 and the beta V(D)J sequence is SEQ ID NO: 4010. In some embodiments, the alpha VJ sequence is SEQ ID NO: 3657 and the beta V(D)J sequence is SEQ ID NO: 3964. In some embodiments, the alpha VJ sequence is SEQ ID NO: 3708 and the beta V(D)J sequence is SEQ ID NO: 4011. In some embodiments, the alpha VJ sequence is SEQ ID NO: 3709 and the beta V(D)J sequence is SEQ ID NO: 4012. In some embodiments, the alpha VJ sequence is SEQ ID NO: 3663 and the beta V(D)J sequence is SEQ ID NO: 3963. In some embodiments, the alpha VJ sequence is SEQ ID NO: 3710 and the beta V(D)J sequence is SEQ ID NO: 4013. In some embodiments, the alpha VJ sequence is SEQ ID NO: 3711 and the beta V(D)J sequence is SEQ ID NO: 4014. In some embodiments, the alpha VJ sequence is SEQ ID NO: 3712 and the beta V(D)J sequence is SEQ ID NO: 4015. In some embodiments, the alpha VJ sequence is SEQ ID NO: 3713 and the beta V(D)J sequence is SEQ ID NO: 4016. In some embodiments, the alpha VJ sequence is SEQ ID NO: 3714 and the beta V(D)J sequence is SEQ ID NO: 4017. In some embodiments, the alpha VJ sequence is SEQ ID NO: 3715 and the beta V(D)J sequence is SEQ ID NO: 4018. In some embodiments, the alpha VJ sequence is SEQ ID NO: 3716 and the beta V(D)J sequence is SEQ ID NO: 4019. In some embodiments, the alpha VJ sequence is SEQ ID NO: 3717 and the beta V(D)J sequence is SEQ ID NO: 4020. In some embodiments, the alpha VJ sequence is SEQ ID NO: 3718 and the beta V(D)J sequence is SEQ ID NO: 4021. In some embodiments, the alpha VJ sequence is SEQ ID NO: 3719 and the beta V(D)J sequence is SEQ ID NO: 4022. In some embodiments, the alpha VJ sequence is SEQ ID NO: 3720 and the beta V(D)J sequence is SEQ ID NO: 4023. In some embodiments, the alpha VJ sequence is SEQ ID NO: 3663 and the beta V(D)J sequence is SEQ ID NO: 3962. In some embodiments, the alpha VJ sequence is SEQ ID NO: 3659 and the beta V(D)J sequence is SEQ ID NO: 3965. In some embodiments, the alpha VJ sequence is SEQ ID NO: 3677 and the beta V(D)J sequence is SEQ ID NO: 4024. In some embodiments, the alpha VJ sequence is SEQ ID NO: 3721 and the beta V(D)J sequence is SEQ ID NO: 4025. In some embodiments, the alpha VJ sequence is SEQ ID NO: 3722 and the beta V(D)J sequence is SEQ ID NO: 4026. In some embodiments, the alpha VJ sequence is SEQ ID NO: 3663 and the beta V(D)J sequence is SEQ ID NO: 3966. In some embodiments, the alpha VJ sequence is SEQ ID NO: 3659 and the beta V(D)J sequence is SEQ ID NO: 4027. In some embodiments, the alpha VJ sequence is SEQ ID NO: 3722 and the beta V(D)J sequence is SEQ ID NO: 3964. In some embodiments, the alpha VJ sequence is SEQ ID NO: 3723 and the beta V(D)J sequence is SEQ ID NO: 4028. In some embodiments, the alpha VJ sequence is SEQ ID NO: 3724 and the beta V(D)J sequence is SEQ ID NO: 4029. In some embodiments, the alpha VJ sequence is SEQ ID NO: 3725 and the beta V(D)J sequence is SEQ ID NO: 4030. In some embodiments, the alpha VJ sequence is SEQ ID NO: 3726 and the beta V(D)J sequence is SEQ ID NO: 4031. In some embodiments, the alpha VJ sequence is SEQ ID NO: 3727 and the beta V(D)J sequence is SEQ ID NO: 4032. In some embodiments, the alpha VJ sequence is SEQ ID NO: 3728 and the beta V(D)J sequence is SEQ ID NO: 4033. In some embodiments, the alpha VJ sequence is SEQ ID NO: 3729 and the beta V(D)J sequence is SEQ ID NO: 4034. In some embodiments, the alpha VJ sequence is SEQ ID NO: 3658 and the beta V(D)J sequence is SEQ ID NO: 3978. In some embodiments, the alpha VJ sequence is SEQ ID NO: 3730 and the beta V(D)J sequence is SEQ ID NO: 4035. In some embodiments, the alpha VJ sequence is SEQ ID NO: 3731 and the beta V(D)J sequence is SEQ ID NO: 4036. In some embodiments, the alpha VJ sequence is SEQ ID NO: 3732 and the beta V(D)J sequence is SEQ ID NO: 4037. In some embodiments, the alpha VJ sequence is SEQ ID NO: 3719 and the beta V(D)J sequence is SEQ ID NO: 3962. In some embodiments, the alpha VJ sequence is SEQ ID NO: 3665 and the beta V(D)J sequence is SEQ ID NO: 3963. In some embodiments, the alpha VJ sequence is SEQ ID NO: 3664 and the beta V(D)J sequence is SEQ ID NO: 3962. In some embodiments, the alpha VJ sequence is SEQ ID NO: 3676 and the beta V(D)J sequence is SEQ ID NO: 3966. In some embodiments, the alpha VJ sequence is SEQ ID NO: 3733 and the beta V(D)J sequence is SEQ ID NO: 4038. In some embodiments, the alpha VJ sequence is SEQ ID NO: 3734 and the beta V(D)J sequence is SEQ ID NO: 4039. In some embodiments, the alpha VJ sequence is SEQ ID NO: 3735 and the beta V(D)J sequence is SEQ ID NO: 4040. In some embodiments, the alpha VJ sequence is SEQ ID NO: 3736 and the beta V(D)J sequence is SEQ ID NO: 4041. In some embodiments, the alpha VJ sequence is SEQ ID NO: 3737 and the beta V(D)J sequence is SEQ ID NO: 4042. In some embodiments, the alpha VJ sequence is SEQ ID NO: 3738 and the beta V(D)J sequence is SEQ ID NO: 3963. In some embodiments, the alpha VJ sequence is SEQ ID NO: 3659 and the beta V(D)J sequence is SEQ ID NO: 3973. In some embodiments, the alpha VJ sequence is SEQ ID NO: 3660 and the beta V(D)J sequence is SEQ ID NO: 3963. In some embodiments, the alpha VJ sequence is SEQ ID NO: 3739 and the beta V(D)J sequence is SEQ ID NO: 4043. In some embodiments, the alpha VJ sequence is SEQ ID NO: 3740 and the beta V(D)J sequence is SEQ ID NO: 4044. In some embodiments, the alpha VJ sequence is SEQ ID NO: 3741 and the beta V(D)J sequence is SEQ ID NO: 4045. In some embodiments, the alpha VJ sequence is SEQ ID NO: 3657 and the beta V(D)J sequence is SEQ ID NO: 3992. In some embodiments, the alpha VJ sequence is SEQ ID NO: 3742 and the beta V(D)J sequence is SEQ ID NO: 4046. In some embodiments, the alpha VJ sequence is SEQ ID NO: 3666 and the beta V(D)J sequence is SEQ ID NO: 3962. In some embodiments, the alpha VJ sequence is SEQ ID NO: 3711 and the beta V(D)J sequence is SEQ ID NO: 3963. In some embodiments, the alpha VJ sequence is SEQ ID NO: 3660 and the beta V(D)J sequence is SEQ ID NO: 3962. In some embodiments, the alpha VJ sequence is SEQ ID NO: 3663 and the beta V(D)J sequence is SEQ ID NO: 3964. In some embodiments, the alpha VJ sequence is SEQ ID NO: 3743 and the beta V(D)J sequence is SEQ ID NO: 4047. In some embodiments, the alpha VJ sequence is SEQ ID NO: 3744 and the beta V(D)J sequence is SEQ ID NO: 4048. In some embodiments, the alpha VJ sequence is SEQ ID NO: 3745 and the beta V(D)J sequence is SEQ ID NO: 4049. In some embodiments, the alpha VJ sequence is SEQ ID NO: 3746 and the beta V(D)J sequence is SEQ ID NO: 4050. In some embodiments, the alpha VJ sequence is SEQ ID NO: 3747 and the beta V(D)J sequence is SEQ ID NO: 4051. In some embodiments, the alpha VJ sequence is SEQ ID NO: 3748 and the beta V(D)J sequence is SEQ ID NO: 4052. In some embodiments, the alpha VJ sequence is SEQ ID NO: 3749 and the beta V(D)J sequence is SEQ ID NO: 4053. In some embodiments, the alpha VJ sequence is SEQ ID NO: 3750 and the beta V(D)J sequence is SEQ ID NO: 4054. In some embodiments, the alpha VJ sequence is SEQ ID NO: 3751 and the beta V(D)J sequence is SEQ ID NO: 4055. In some embodiments, the alpha VJ sequence is SEQ ID NO: 3752 and the beta V(D)J sequence is SEQ ID NO: 4056. In some embodiments, the alpha VJ sequence is SEQ ID NO: 3753 and the beta V(D)J sequence is SEQ ID NO: 4057. In some embodiments, the alpha VJ sequence is SEQ ID NO: 3754 and the beta V(D)J sequence is SEQ ID NO: 4058. In some embodiments, the alpha VJ sequence is SEQ ID NO: 3755 and the beta V(D)J sequence is SEQ ID NO: 4057. In some embodiments, the alpha VJ sequence is SEQ ID NO: 3756 and the beta V(D)J sequence is SEQ ID NO: 4059. In some embodiments, the alpha VJ sequence is SEQ ID NO: 3757 and the beta V(D)J sequence is SEQ ID NO: 4060. In some embodiments, the alpha VJ sequence is SEQ ID NO: 3758 and the beta V(D)J sequence is SEQ ID NO: 4061. In some embodiments, the alpha VJ sequence is SEQ ID NO: 3759 and the beta V(D)J sequence is SEQ ID NO: 4062. In some embodiments, the alpha VJ sequence is SEQ ID NO: 3760 and the beta V(D)J sequence is SEQ ID NO: 4063. In some embodiments, the alpha VJ sequence is SEQ ID NO: 3761 and the beta V(D)J sequence is SEQ ID NO: 4049. In some embodiments, the alpha VJ sequence is SEQ ID NO: 3748 and the beta V(D)J sequence is SEQ ID NO: 4049. In some embodiments, the alpha VJ sequence is SEQ ID NO: 3762 and the beta V(D)J sequence is SEQ ID NO: 4064. In some embodiments, the alpha VJ sequence is SEQ ID NO: 3763 and the beta V(D)J sequence is SEQ ID NO: 4065. In some embodiments, the alpha VJ sequence is SEQ ID NO: 3764 and the beta V(D)J sequence is SEQ ID NO: 4066. In some embodiments, the alpha VJ sequence is SEQ ID NO: 3765 and the beta V(D)J sequence is SEQ ID NO: 4067. In some embodiments, the alpha VJ sequence is SEQ ID NO: 3746 and the beta V(D)J sequence is SEQ ID NO: 4053. In some embodiments, the alpha VJ sequence is SEQ ID NO: 3766 and the beta V(D)J sequence is SEQ ID NO: 4068. In some embodiments, the alpha VJ sequence is SEQ ID NO: 3761 and the beta V(D)J sequence is SEQ ID NO: 4069. In some embodiments, the alpha VJ sequence is SEQ ID NO: 3767 and the beta V(D)J sequence is SEQ ID NO: 4070. In some embodiments, the alpha VJ sequence is SEQ ID NO: 3768 and the beta V(D)J sequence is SEQ ID NO: 4071. In some embodiments, the alpha VJ sequence is SEQ ID NO: 3769 and the beta V(D)J sequence is SEQ ID NO: 4072. In some embodiments, the alpha VJ sequence is SEQ ID NO: 3770 and the beta V(D)J sequence is SEQ ID NO: 4073. In some embodiments, the alpha VJ sequence is SEQ ID NO: 3771 and the beta V(D)J sequence is SEQ ID NO: 4074. In some embodiments, the alpha VJ sequence is SEQ ID NO: 3772 and the beta V(D)J sequence is SEQ ID NO: 4075. In some embodiments, the alpha VJ sequence is SEQ ID NO: 3773 and the beta V(D)J sequence is SEQ ID NO: 4076. In some embodiments, the alpha VJ sequence is SEQ ID NO: 3774 and the beta V(D)J sequence is SEQ ID NO: 4077. In some embodiments, the alpha VJ sequence is SEQ ID NO: 3775 and the beta V(D)J sequence is SEQ ID NO: 4078. In some embodiments, the alpha VJ sequence is SEQ ID NO: 3746 and the beta V(D)J sequence is SEQ ID NO: 4055. In some embodiments, the alpha VJ sequence is SEQ ID NO: 3745 and the beta V(D)J sequence is SEQ ID NO: 4051. In some embodiments, the alpha VJ sequence is SEQ ID NO: 3776 and the beta V(D)J sequence is SEQ ID NO: 4079. In some embodiments, the alpha VJ sequence is SEQ ID NO: 3777 and the beta V(D)J sequence is SEQ ID NO: 4080. In some embodiments, the alpha VJ sequence is SEQ ID NO: 3778 and the beta V(D)J sequence is SEQ ID NO: 4081. In some embodiments, the alpha VJ sequence is SEQ ID NO: 3779 and the beta V(D)J sequence is SEQ ID NO: 4082. In some embodiments, the alpha VJ sequence is SEQ ID NO: 3780 and the beta V(D)J sequence is SEQ ID NO: 4083. In some embodiments, the alpha VJ sequence is SEQ ID NO: 3746 and the beta V(D)J sequence is SEQ ID NO: 4049. In some embodiments, the alpha VJ sequence is SEQ ID NO: 3781 and the beta V(D)J sequence is SEQ ID NO: 4084. In some embodiments, the alpha VJ sequence is SEQ ID NO: 3782 and the beta V(D)J sequence is SEQ ID NO: 4085. In some embodiments, the alpha VJ sequence is SEQ ID NO: 3783 and the beta V(D)J sequence is SEQ ID NO: 4086. In some embodiments, the alpha VJ sequence is SEQ ID NO: 3784 and the beta V(D)J sequence is SEQ ID NO: 4087. In some embodiments, the alpha VJ sequence is SEQ ID NO: 3785 and the beta V(D)J sequence is SEQ ID NO: 4088. In some embodiments, the alpha VJ sequence is SEQ ID NO: 3786 and the beta V(D)J sequence is SEQ ID NO: 4089. In some embodiments, the alpha VJ sequence is SEQ ID NO: 3787 and the beta V(D)J sequence is SEQ ID NO: 4090. In some embodiments, the alpha VJ sequence is SEQ ID NO: 3788 and the beta V(D)J sequence is SEQ ID NO: 4091. In some embodiments, the alpha VJ sequence is SEQ ID NO: 3789 and the beta V(D)J sequence is SEQ ID NO: 4092. In some embodiments, the alpha VJ sequence is SEQ ID NO: 3790 and the beta V(D)J sequence is SEQ ID NO: 4093. In some embodiments, the alpha VJ sequence is SEQ ID NO: 3791 and the beta V(D)J sequence is SEQ ID NO: 4094. In some embodiments, the alpha VJ sequence is SEQ ID NO: 3755 and the beta V(D)J sequence is SEQ ID NO: 4095. In some embodiments, the alpha VJ sequence is SEQ ID NO: 3792 and the beta V(D)J sequence is SEQ ID NO: 4096. In some embodiments, the alpha VJ sequence is SEQ ID NO: 3793 and the beta V(D)J sequence is SEQ ID NO: 4097. In some embodiments, the alpha VJ sequence is SEQ ID NO: 3794 and the beta V(D)J sequence is SEQ ID NO: 4098. In some embodiments, the alpha VJ sequence is SEQ ID NO: 3795 and the beta V(D)J sequence is SEQ ID NO: 4099. In some embodiments, the alpha VJ sequence is SEQ ID NO: 3796 and the beta V(D)J sequence is SEQ ID NO: 4100. In some embodiments, the alpha VJ sequence is SEQ ID NO: 3797 and the beta V(D)J sequence is SEQ ID NO: 4101. In some embodiments, the alpha VJ sequence is SEQ ID NO: 3798 and the beta V(D)J sequence is SEQ ID NO: 4102. In some embodiments, the alpha VJ sequence is SEQ ID NO: 3799 and the beta V(D)J sequence is SEQ ID NO: 4095. In some embodiments, the alpha VJ sequence is SEQ ID NO: 3800 and the beta V(D)J sequence is SEQ ID NO: 4103. In some embodiments, the alpha VJ sequence is SEQ ID NO: 3801 and the beta V(D)J sequence is SEQ ID NO: 4104. In some embodiments, the alpha VJ sequence is SEQ ID NO: 3802 and the beta V(D)J sequence is SEQ ID NO: 4105. In some embodiments, the alpha VJ sequence is SEQ ID NO: 3803 and the beta V(D)J sequence is SEQ ID NO: 4106. In some embodiments, the alpha VJ sequence is SEQ ID NO: 3804 and the beta V(D)J sequence is SEQ ID NO: 4107. In some embodiments, the alpha VJ sequence is SEQ ID NO: 3805 and the beta V(D)J sequence is SEQ ID NO: 4108. In some embodiments, the alpha VJ sequence is SEQ ID NO: 3806 and the beta V(D)J sequence is SEQ ID NO: 4109. In some embodiments, the alpha VJ sequence is SEQ ID NO: 3807 and the beta V(D)J sequence is SEQ ID NO: 4110. In some embodiments, the alpha VJ sequence is SEQ ID NO: 3808 and the beta V(D)J sequence is SEQ ID NO: 4111. In some embodiments, the alpha VJ sequence is SEQ ID NO: 3809 and the beta V(D)J sequence is SEQ ID NO: 4112. In some embodiments, the alpha VJ sequence is SEQ ID NO: 3810 and the beta V(D)J sequence is SEQ ID NO: 4113. In some embodiments, the alpha VJ sequence is SEQ ID NO: 3746 and the beta V(D)J sequence is SEQ ID NO: 4062. In some embodiments, the alpha VJ sequence is SEQ ID NO: 3811 and the beta V(D)J sequence is SEQ ID NO: 4049. In some embodiments, the alpha VJ sequence is SEQ ID NO: 3812 and the beta V(D)J sequence is SEQ ID NO: 4114. In some embodiments, the alpha VJ sequence is SEQ ID NO: 3747 and the beta V(D)J sequence is SEQ ID NO: 4049. In some embodiments, the alpha VJ sequence is SEQ ID NO: 3813 and the beta V(D)J sequence is SEQ ID NO: 4115. In some embodiments, the alpha VJ sequence is SEQ ID NO: 3814 and the beta V(D)J sequence is SEQ ID NO: 4116. In some embodiments, the alpha VJ sequence is SEQ ID NO: 3815 and the beta V(D)J sequence is SEQ ID NO: 4117. In some embodiments, the alpha VJ sequence is SEQ ID NO: 3816 and the beta V(D)J sequence is SEQ ID NO: 4118. In some embodiments, the alpha VJ sequence is SEQ ID NO: 3817 and the beta V(D)J sequence is SEQ ID NO: 4119. In some embodiments, the alpha VJ sequence is SEQ ID NO: 3818 and the beta V(D)J sequence is SEQ ID NO: 4120. In some embodiments, the alpha VJ sequence is SEQ ID NO: 3758 and the beta V(D)J sequence is SEQ ID NO: 4053. In some embodiments, the alpha VJ sequence is SEQ ID NO: 3819 and the beta V(D)J sequence is SEQ ID NO: 4121. In some embodiments, the alpha VJ sequence is SEQ ID NO: 3820 and the beta V(D)J sequence is SEQ ID NO: 4122. In some embodiments, the alpha VJ sequence is SEQ ID NO: 3821 and the beta V(D)J sequence is SEQ ID NO: 4123. In some embodiments, the alpha VJ sequence is SEQ ID NO: 3822 and the beta V(D)J sequence is SEQ ID NO: 4124. In some embodiments, the alpha VJ sequence is SEQ ID NO: 3823 and the beta V(D)J sequence is SEQ ID NO: 4125. In some embodiments, the alpha VJ sequence is SEQ ID NO: 3824 and the beta V(D)J sequence is SEQ ID NO: 4126. In some embodiments, the alpha VJ sequence is SEQ ID NO: 3825 and the beta V(D)J sequence is SEQ ID NO: 4127. In some embodiments, the alpha VJ sequence is SEQ ID NO: 3826 and the beta V(D)J sequence is SEQ ID NO: 4128. In some embodiments, the alpha VJ sequence is SEQ ID NO: 3827 and the beta V(D)J sequence is SEQ ID NO: 4129. In some embodiments, the alpha VJ sequence is SEQ ID NO: 3828 and the beta V(D)J sequence is SEQ ID NO: 4130. In some embodiments, the alpha VJ sequence is SEQ ID NO: 3829 and the beta V(D)J sequence is SEQ ID NO: 4131. In some embodiments, the alpha VJ sequence is SEQ ID NO: 3830 and the beta V(D)J sequence is SEQ ID NO: 4132. In some embodiments, the alpha VJ sequence is SEQ ID NO: 3831 and the beta V(D)J sequence is SEQ ID NO: 4133. In some embodiments, the alpha VJ sequence is SEQ ID NO: 3832 and the beta V(D)J sequence is SEQ ID NO: 4134. In some embodiments, the alpha VJ sequence is SEQ ID NO: 3828 and the beta V(D)J sequence is SEQ ID NO: 4131. In some embodiments, the alpha VJ sequence is SEQ ID NO: 3833 and the beta V(D)J sequence is SEQ ID NO: 4135. In some embodiments, the alpha VJ sequence is SEQ ID NO: 3834 and the beta V(D)J sequence is SEQ ID NO: 4136. In some embodiments, the alpha VJ sequence is SEQ ID NO: 3835 and the beta V(D)J sequence is SEQ ID NO: 4137. In some embodiments, the alpha VJ sequence is SEQ ID NO: 3836 and the beta V(D)J sequence is SEQ ID NO: 4138. In some embodiments, the alpha VJ sequence is SEQ ID NO: 3837 and the beta V(D)J sequence is SEQ ID NO: 4139. In some embodiments, the alpha VJ sequence is SEQ ID NO: 3838 and the beta V(D)J sequence is SEQ ID NO: 4140. In some embodiments, the alpha VJ sequence is SEQ ID NO: 3839 and the beta V(D)J sequence is SEQ ID NO: 4141. In some embodiments, the alpha VJ sequence is SEQ ID NO: 3840 and the beta V(D)J sequence is SEQ ID NO: 4142. In some embodiments, the alpha VJ sequence is SEQ ID NO: 3841 and the beta V(D)J sequence is SEQ ID NO: 4143. In some embodiments, the alpha VJ sequence is SEQ ID NO: 3828 and the beta V(D)J sequence is SEQ ID NO: 4138. In some embodiments, the alpha VJ sequence is SEQ ID NO: 3842 and the beta V(D)J sequence is SEQ ID NO: 4144. In some embodiments, the alpha VJ sequence is SEQ ID NO: 3843 and the beta V(D)J sequence is SEQ ID NO: 4145. In some embodiments, the alpha VJ sequence is SEQ ID NO: 3844 and the beta V(D)J sequence is SEQ ID NO: 4133. In some embodiments, the alpha VJ sequence is SEQ ID NO: 3828 and the beta V(D)J sequence is SEQ ID NO: 4143. In some embodiments, the alpha VJ sequence is SEQ ID NO: 3845 and the beta V(D)J sequence is SEQ ID NO: 4146. In some embodiments, the alpha VJ sequence is SEQ ID NO: 3846 and the beta V(D)J sequence is SEQ ID NO: 4147. In some embodiments, the alpha VJ sequence is SEQ ID NO: 3828 and the beta V(D)J sequence is SEQ ID NO: 4145. In some embodiments, the alpha VJ sequence is SEQ ID NO: 3847 and the beta V(D)J sequence is SEQ ID NO: 4148. In some embodiments, the alpha VJ sequence is SEQ ID NO: 3848 and the beta V(D)J sequence is SEQ ID NO: 4149. In some embodiments, the alpha VJ sequence is SEQ ID NO: 3849 and the beta V(D)J sequence is SEQ ID NO: 4150. In some embodiments, the alpha VJ sequence is SEQ ID NO: 3850 and the beta V(D)J sequence is SEQ ID NO: 4151. In some embodiments, the alpha VJ sequence is SEQ ID NO: 3851 and the beta V(D)J sequence is SEQ ID NO: 4152. In some embodiments, the alpha VJ sequence is SEQ ID NO: 3852 and the beta V(D)J sequence is SEQ ID NO: 4153. In some embodiments, the alpha VJ sequence is SEQ ID NO: 3853 and the beta V(D)J sequence is SEQ ID NO: 4154. In some embodiments, the alpha VJ sequence is SEQ ID NO: 3854 and the beta V(D)J sequence is SEQ ID NO: 4155. In some embodiments, the alpha VJ sequence is SEQ ID NO: 3855 and the beta V(D)J sequence is SEQ ID NO: 4156. In some embodiments, the alpha VJ sequence is SEQ ID NO: 3831 and the beta V(D)J sequence is SEQ ID NO: 4157. In some embodiments, the alpha VJ sequence is SEQ ID NO: 3856 and the beta V(D)J sequence is SEQ ID NO: 4158. In some embodiments, the alpha VJ sequence is SEQ ID NO: 3857 and the beta V(D)J sequence is SEQ ID NO: 4159. In some embodiments, the alpha VJ sequence is SEQ ID NO: 3858 and the beta V(D)J sequence is SEQ ID NO: 4160. In some embodiments, the alpha VJ sequence is SEQ ID NO: 3859 and the beta V(D)J sequence is SEQ ID NO: 4161. In some embodiments, the alpha VJ sequence is SEQ ID NO: 3828 and the beta V(D)J sequence is SEQ ID NO: 4137. In some embodiments, the alpha VJ sequence is SEQ ID NO: 3860 and the beta V(D)J sequence is SEQ ID NO: 4162. In some embodiments, the alpha VJ sequence is SEQ ID NO: 3861 and the beta V(D)J sequence is SEQ ID NO: 4163. In some embodiments, the alpha VJ sequence is SEQ ID NO: 3862 and the beta V(D)J sequence is SEQ ID NO: 4164. In some embodiments, the alpha VJ sequence is SEQ ID NO: 3863 and the beta V(D)J sequence is SEQ ID NO: 4165. In some embodiments, the alpha VJ sequence is SEQ ID NO: 3864 and the beta V(D)J sequence is SEQ ID NO: 4166. In some embodiments, the alpha VJ sequence is SEQ ID NO: 3865 and the beta V(D)J sequence is SEQ ID NO: 4167. In some embodiments, the alpha VJ sequence is SEQ ID NO: 3866 and the beta V(D)J sequence is SEQ ID NO: 4168. In some embodiments, the alpha VJ sequence is SEQ ID NO: 3867 and the beta V(D)J sequence is SEQ ID NO: 4169. In some embodiments, the alpha VJ sequence is SEQ ID NO: 3868 and the beta V(D)J sequence is SEQ ID NO: 4170. In some embodiments, the alpha VJ sequence is SEQ ID NO: 3869 and the beta V(D)J sequence is SEQ ID NO: 4171. In some embodiments, the alpha VJ sequence is SEQ ID NO: 3870 and the beta V(D)J sequence is SEQ ID NO: 4172. In some embodiments, the alpha VJ sequence is SEQ ID NO: 3871 and the beta V(D)J sequence is SEQ ID NO: 4173. In some embodiments, the alpha VJ sequence is SEQ ID NO: 3828 and the beta V(D)J sequence is SEQ ID NO: 4132. In some embodiments, the alpha VJ sequence is SEQ ID NO: 3872 and the beta V(D)J sequence is SEQ ID NO: 4174. In some embodiments, the alpha VJ sequence is SEQ ID NO: 3873 and the beta V(D)J sequence is SEQ ID NO: 4175. In some embodiments, the alpha VJ sequence is SEQ ID NO: 3828 and the beta V(D)J sequence is SEQ ID NO: 4176. In some embodiments, the alpha VJ sequence is SEQ ID NO: 3874 and the beta V(D)J sequence is SEQ ID NO: 4177. In some embodiments, the alpha VJ sequence is SEQ ID NO: 3875 and the beta V(D)J sequence is SEQ ID NO: 4178. In some embodiments, the alpha VJ sequence is SEQ ID NO: 3876 and the beta V(D)J sequence is SEQ ID NO: 4179. In some embodiments, the alpha VJ sequence is SEQ ID NO: 3877 and the beta V(D)J sequence is SEQ ID NO: 4180. In some embodiments, the alpha VJ sequence is SEQ ID NO: 3878 and the beta V(D)J sequence is SEQ ID NO: 4181. In some embodiments, the alpha VJ sequence is SEQ ID NO: 3879 and the beta V(D)J sequence is SEQ ID NO: 4182. In some embodiments, the alpha VJ sequence is SEQ ID NO: 3828 and the beta V(D)J sequence is SEQ ID NO: 4141. In some embodiments, the alpha VJ sequence is SEQ ID NO: 3880 and the beta V(D)J sequence is SEQ ID NO: 4183. In some embodiments, the alpha VJ sequence is SEQ ID NO: 3828 and the beta V(D)J sequence is SEQ ID NO: 4184. In some embodiments, the alpha VJ sequence is SEQ ID NO: 3881 and the beta V(D)J sequence is SEQ ID NO: 4185. In some embodiments, the alpha VJ sequence is SEQ ID NO: 3830 and the beta V(D)J sequence is SEQ ID NO: 4135. In some embodiments, the alpha VJ sequence is SEQ ID NO: 3882 and the beta V(D)J sequence is SEQ ID NO: 4186. In some embodiments, the alpha VJ sequence is SEQ ID NO: 3883 and the beta V(D)J sequence is SEQ ID NO: 4187. In some embodiments, the alpha VJ sequence is SEQ ID NO: 3884 and the beta V(D)J sequence is SEQ ID NO: 4188. In some embodiments, the alpha VJ sequence is SEQ ID NO: 3885 and the beta V(D)J sequence is SEQ ID NO: 4189. In some embodiments, the alpha VJ sequence is SEQ ID NO: 3828 and the beta V(D)J sequence is SEQ ID NO: 4190. In some embodiments, the alpha VJ sequence is SEQ ID NO: 3841 and the beta V(D)J sequence is SEQ ID NO: 4130. In some embodiments, the alpha VJ sequence is SEQ ID NO: 3886 and the beta V(D)J sequence is SEQ ID NO: 4191. In some embodiments, the alpha VJ sequence is SEQ ID NO: 3887 and the beta V(D)J sequence is SEQ ID NO: 4192. In some embodiments, the alpha VJ sequence is SEQ ID NO: 3888 and the beta V(D)J sequence is SEQ ID NO: 4193. In some embodiments, the alpha VJ sequence is SEQ ID NO: 3889 and the beta V(D)J sequence is SEQ ID NO: 4194. In some embodiments, the alpha VJ sequence is SEQ ID NO: 3890 and the beta V(D)J sequence is SEQ ID NO: 4195. In some embodiments, the alpha VJ sequence is SEQ ID NO: 3891 and the beta V(D)J sequence is SEQ ID NO: 4196. In some embodiments, the alpha VJ sequence is SEQ ID NO: 3892 and the beta V(D)J sequence is SEQ ID NO: 4197. In some embodiments, the alpha VJ sequence is SEQ ID NO: 3893 and the beta V(D)J sequence is SEQ ID NO: 4198. In some embodiments, the alpha VJ sequence is SEQ ID NO: 3894 and the beta V(D)J sequence is SEQ ID NO: 4199. In some embodiments, the alpha VJ sequence is SEQ ID NO: 3895 and the beta V(D)J sequence is SEQ ID NO: 4200. In some embodiments, the alpha VJ sequence is SEQ ID NO: 3896 and the beta V(D)J sequence is SEQ ID NO: 4201. In some embodiments, the alpha VJ sequence is SEQ ID NO: 3897 and the beta V(D)J sequence is SEQ ID NO: 4202. In some embodiments, the alpha VJ sequence is SEQ ID NO: 3898 and the beta V(D)J sequence is SEQ ID NO: 4203. In some embodiments, the alpha VJ sequence is SEQ ID NO: 3899 and the beta V(D)J sequence is SEQ ID NO: 4204. In some embodiments, the alpha VJ sequence is SEQ ID NO: 3900 and the beta V(D)J sequence is SEQ ID NO: 4205. In some embodiments, the alpha VJ sequence is SEQ ID NO: 3901 and the beta V(D)J sequence is SEQ ID NO: 4206. In some embodiments, the alpha VJ sequence is SEQ ID NO: 3902 and the beta V(D)J sequence is SEQ ID NO: 4207. In some embodiments, the alpha VJ sequence is SEQ ID NO: 3903 and the beta V(D)J sequence is SEQ ID NO: 4208. In some embodiments, the alpha VJ sequence is SEQ ID NO: 3904 and the beta V(D)J sequence is SEQ ID NO: 4209. In some embodiments, the alpha VJ sequence is SEQ ID NO: 3905 and the beta V(D)J sequence is SEQ ID NO: 4210. In some embodiments, the alpha VJ sequence is SEQ ID NO: 3830 and the beta V(D)J sequence is SEQ ID NO: 4211. In some embodiments, the alpha VJ sequence is SEQ ID NO: 3906 and the beta V(D)J sequence is SEQ ID NO: 4212. In some embodiments, the alpha VJ sequence is SEQ ID NO: 3828 and the beta V(D)J sequence is SEQ ID NO: 4213. In some embodiments, the alpha VJ sequence is SEQ ID NO: 3907 and the beta V(D)J sequence is SEQ ID NO: 4214. In some embodiments, the alpha VJ sequence is SEQ ID NO: 3908 and the beta V(D)J sequence is SEQ ID NO: 4215. In some embodiments, the alpha VJ sequence is SEQ ID NO: 3909 and the beta V(D)J sequence is SEQ ID NO: 4216. In some embodiments, the alpha VJ sequence is SEQ ID NO: 3910 and the beta V(D)J sequence is SEQ ID NO: 4217. In some embodiments, the alpha VJ sequence is SEQ ID NO: 3911 and the beta V(D)J sequence is SEQ ID NO: 4218. In some embodiments, the alpha VJ sequence is SEQ ID NO: 3912 and the beta V(D)J sequence is SEQ ID NO: 4219. In some embodiments, the alpha VJ sequence is SEQ ID NO: 3913 and the beta V(D)J sequence is SEQ ID NO: 4220. In some embodiments, the alpha VJ sequence is SEQ ID NO: 3914 and the beta V(D)J sequence is SEQ ID NO: 4221. In some embodiments, the alpha VJ sequence is SEQ ID NO: 3915 and the beta V(D)J sequence is SEQ ID NO: 4222. In some embodiments, the alpha VJ sequence is SEQ ID NO: 3916 and the beta V(D)J sequence is SEQ ID NO: 4223. In some embodiments, the alpha VJ sequence is SEQ ID NO: 3917 and the beta V(D)J sequence is SEQ ID NO: 4224. In some embodiments, the alpha VJ sequence is SEQ ID NO: 3899 and the beta V(D)J sequence is SEQ ID NO: 4208. In some embodiments, the alpha VJ sequence is SEQ ID NO: 3918 and the beta V(D)J sequence is SEQ ID NO: 4225. In some embodiments, the alpha VJ sequence is SEQ ID NO: 3919 and the beta V(D)J sequence is SEQ ID NO: 4226. In some embodiments, the alpha VJ sequence is SEQ ID NO: 3920 and the beta V(D)J sequence is SEQ ID NO: 4227. In some embodiments, the alpha VJ sequence is SEQ ID NO: 3921 and the beta V(D)J sequence is SEQ ID NO: 4228. In some embodiments, the alpha VJ sequence is SEQ ID NO: 3922 and the beta V(D)J sequence is SEQ ID NO: 4229. In some embodiments, the alpha VJ sequence is SEQ ID NO: 3923 and the beta V(D)J sequence is SEQ ID NO: 4230. In some embodiments, the alpha VJ sequence is SEQ ID NO: 3924 and the beta V(D)J sequence is SEQ ID NO: 4231. In some embodiments, the alpha VJ sequence is SEQ ID NO: 3899 and the beta V(D)J sequence is SEQ ID NO: 4232. In some embodiments, the alpha VJ sequence is SEQ ID NO: 3925 and the beta V(D)J sequence is SEQ ID NO: 4233. In some embodiments, the alpha VJ sequence is SEQ ID NO: 3926 and the beta V(D)J sequence is SEQ ID NO: 4234. In some embodiments, the alpha VJ sequence is SEQ ID NO: 3927 and the beta V(D)J sequence is SEQ ID NO: 4235. In some embodiments, the alpha VJ sequence is SEQ ID NO: 3928 and the beta V(D)J sequence is SEQ ID NO: 4236. In some embodiments, the alpha VJ sequence is SEQ ID NO: 3929 and the beta V(D)J sequence is SEQ ID NO: 4237. In some embodiments, the alpha VJ sequence is SEQ ID NO: 3930 and the beta V(D)J sequence is SEQ ID NO: 4238. In some embodiments, the alpha VJ sequence is SEQ ID NO: 3931 and the beta V(D)J sequence is SEQ ID NO: 4239. In some embodiments, the alpha VJ sequence is SEQ ID NO: 3932 and the beta V(D)J sequence is SEQ ID NO: 4240. In some embodiments, the alpha VJ sequence is SEQ ID NO: 3933 and the beta V(D)J sequence is SEQ ID NO: 4241. In some embodiments, the alpha VJ sequence is SEQ ID NO: 3934 and the beta V(D)J sequence is SEQ ID NO: 4242. In some embodiments, the alpha VJ sequence is SEQ ID NO: 3935 and the beta V(D)J sequence is SEQ ID NO: 4215. In some embodiments, the alpha VJ sequence is SEQ ID NO: 3936 and the beta V(D)J sequence is SEQ ID NO: 4243. In some embodiments, the alpha VJ sequence is SEQ ID NO: 3937 and the beta V(D)J sequence is SEQ ID NO: 4244. In some embodiments, the alpha VJ sequence is SEQ ID NO: 3938 and the beta V(D)J sequence is SEQ ID NO: 4245. In some embodiments, the alpha VJ sequence is SEQ ID NO: 3899 and the beta V(D)J sequence is SEQ ID NO: 4211. In some embodiments, the alpha VJ sequence is SEQ ID NO: 3939 and the beta V(D)J sequence is SEQ ID NO: 4246. In some embodiments, the alpha VJ sequence is SEQ ID NO: 3940 and the beta V(D)J sequence is SEQ ID NO: 4247. In some embodiments, the alpha VJ sequence is SEQ ID NO: 3941 and the beta V(D)J sequence is SEQ ID NO: 4248. In some embodiments, the alpha VJ sequence is SEQ ID NO: 3942 and the beta V(D)J sequence is SEQ ID NO: 4249. In some embodiments, the alpha VJ sequence is SEQ ID NO: 3943 and the beta V(D)J sequence is SEQ ID NO: 4250. In some embodiments, the alpha VJ sequence is SEQ ID NO: 3944 and the beta V(D)J sequence is SEQ ID NO: 4251. In some embodiments, the alpha VJ sequence is SEQ ID NO: 3945 and the beta V(D)J sequence is SEQ ID NO: 4252. In some embodiments, the alpha VJ sequence is SEQ ID NO: 3946 and the beta V(D)J sequence is SEQ ID NO: 4253. In some embodiments, the alpha VJ sequence is SEQ ID NO: 3947 and the beta V(D)J sequence is SEQ ID NO: 4254. In some embodiments, the alpha VJ sequence is SEQ ID NO: 3948 and the beta V(D)J sequence is SEQ ID NO: 4255. In some embodiments, the alpha VJ sequence is SEQ ID NO: 3900 and the beta V(D)J sequence is SEQ ID NO: 4209. In some embodiments, the alpha VJ sequence is SEQ ID NO: 3949 and the beta V(D)J sequence is SEQ ID NO: 4256. In some embodiments, the alpha VJ sequence is SEQ ID NO: 3900 and the beta V(D)J sequence is SEQ ID NO: 4214. In some embodiments, the alpha VJ sequence is SEQ ID NO: 3950 and the beta V(D)J sequence is SEQ ID NO: 4257. In some embodiments, the alpha VJ sequence is SEQ ID NO: 3899 and the beta V(D)J sequence is SEQ ID NO: 4224. In some embodiments, the alpha VJ sequence is SEQ ID NO: 3951 and the beta V(D)J sequence is SEQ ID NO: 4258. In some embodiments, the alpha VJ sequence is SEQ ID NO: 3952 and the beta V(D)J sequence is SEQ ID NO: 4259. In some embodiments, the alpha VJ sequence is SEQ ID NO: 3953 and the beta V(D)J sequence is SEQ ID NO: 4260. In some embodiments, the alpha VJ sequence is SEQ ID NO: 3751 and the beta V(D)J sequence is SEQ ID NO: 4261. In some embodiments, the alpha VJ sequence is SEQ ID NO: 3954 and the beta V(D)J sequence is SEQ ID NO: 4262. In some embodiments, the alpha VJ sequence is SEQ ID NO: 3955 and the beta V(D)J sequence is SEQ ID NO: 4263. In some embodiments, the alpha VJ sequence is SEQ ID NO: 3956 and the beta V(D)J sequence is SEQ ID NO: 4264. In some embodiments, the alpha VJ sequence is SEQ ID NO: 3957 and the beta V(D)J sequence is SEQ ID NO: 4265. In some embodiments, the alpha VJ sequence is SEQ ID NO: 3958 and the beta V(D)J sequence is SEQ ID NO: 4266. In some embodiments, the alpha VJ sequence is SEQ ID NO: 3959 and the beta V(D)J sequence is SEQ ID NO: 4267. In some embodiments, the alpha VJ sequence is SEQ ID NO: 3960 and the beta V(D)J sequence is SEQ ID NO: 4268. In some embodiments, the alpha VJ sequence is SEQ ID NO: 3961 and the beta V(D)J sequence is SEQ ID NO: 4269. In some embodiments, the alpha VJ sequence is SEQ ID NO: 4302 and the beta V(D)J sequence is SEQ ID NO: 4317. In some embodiments, the alpha VJ sequence is SEQ ID NO: 4303 and the beta V(D)J sequence is SEQ ID NO: 4318. In some embodiments, the alpha VJ sequence is SEQ ID NO: 4304 and the beta V(D)J sequence is SEQ ID NO: 4319. In some embodiments, the alpha VJ sequence is SEQ ID NO: 4305 and the beta V(D)J sequence is SEQ ID NO: 4320.

Target-Specific TCRs to B *44:02 GEMSSNSTAL (SEQ ID NO: 4272)

In some aspects, provided herein are ABPs comprising TCRs or antigen-binding fragments thereof that specifically bind an HLA-PEPTIDE target, wherein the HLA Class I molecule of the HLA-PEPTIDE target is HLA subtype B*44:02 and the HLA-restricted peptide of the HLA-PEPTIDE target comprises the sequence GEMSSNSTAL (SEQ ID NO: 4272).

The TCR specific for B*44:02_GEMSSNSTAL (SEQ ID NO: 4272) may comprise an αCDR3 sequence. The αCDR3 sequence may be any one of SEQ ID NOS 4284-4286 or 3138.

The TCR specific for B*44:02_GEMSSNSTAL (SEQ ID NO: 4272) may comprise a βCDR3 sequence. The βCDR3 sequence may be any one of SEQ ID NOS 4298-4301.

The TCR specific for B*44:02_GEMSSNSTAL (SEQ ID NO: 4272) may comprise a particular αCDR3 sequence and a particular βCDR3 sequence. The αCDR3 may be SEQ ID NO: 4284 and the βCDR3 may be SEQ ID NO: 4298. The αCDR3 may be SEQ ID NO: 4285 and the βCDR3 may be SEQ ID NO: 4299. The αCDR3 may be SEQ ID NO: 4286 and the βCDR3 may be SEQ ID NO: 4300. The αCDR3 may be SEQ ID NO: 3138 and the βCDR3 may be SEQ ID NO: 4301.

The TCR specific for B*44:02_GEMSSNSTAL (SEQ ID NO: 4272) may comprise a TRAV, a TRAJ, a TRBV, optionally a TRBD, and a TRBJ amino acid sequence, optionally a TRAC sequence and optionally a TRBC sequence. Such TCR may comprise TRAV19, TRAJ39, TRBV7-6, TRBD1, and TRBJ1-1. Such TCR may comprise TRAV36DV7, TRAJ34, TRBV7-6, TRBD2, and TRBJ2-2. Such TCR may comprise TRAV24, TRAJ15, TRBV7-6, TRBD2, and TRBJ2-1. Such TCR may comprise TRAV8-4, TRAJ12, TRBV12-4, TRBD2, and TRBJ2-3.

The TCR specific for B*44:02_GEMSSNSTAL (SEQ ID NO: 4272) may comprise an alpha VJ sequence. The alpha VJ sequence may be any one of SEQ ID NOS 4313-4316.

The TCR specific for B*44:02_GEMSSNSTAL (SEQ ID NO: 4272) may comprise a beta V(D)J sequence. The beta V(D)J sequence may be any one of SEQ ID NOS 4328-4331.

In some embodiments, the alpha VJ sequence is SEQ ID NO: 4313 and the beta V(D)J sequence is SEQ ID NO: 4328. In some embodiments, the alpha VJ sequence is SEQ ID NO: 4314 and the beta V(D)J sequence is SEQ ID NO: 4329. In some embodiments, the alpha VJ sequence is SEQ ID NO: 4315 and the beta V(D)J sequence is SEQ ID NO: 4330. In some embodiments, the alpha VJ sequence is SEQ ID NO: 4316 and the beta V(D)J sequence is SEQ ID NO: 4331.

Target-Specific TCRs to A *02:01_GVYDGEEHSV (SEQ ID NO: 4271)

In some aspects, provided herein are ABPs comprising TCRs or antigen-binding fragments thereof that specifically bind an HLA-PEPTIDE target, wherein the HLA Class I molecule of the HLA-PEPTIDE target is HLA subtype A*02:01 and the HLA-restricted peptide of the HLA-PEPTIDE target comprises the sequence GVYDGEEHSV (SEQ ID NO: 4271).

The TCR specific for A*02:01_GVYDGEEHSV (SEQ ID NO: 4271) may comprise an αCDR3 sequence. The αCDR3 sequence may be any one of SEQ ID NOS 4282-4283.

The TCR specific for A*02:01_GVYDGEEHSV (SEQ ID NO: 4271) may comprise a βCDR3 sequence. The βCDR3 sequence may be any one of SEQ ID NOS 4296-4297.

The TCR specific for A*02:01_GVYDGEEHSV (SEQ ID NO: 4271) may comprise a particular αCDR3 sequence and a particular βCDR3 sequence. The αCDR3 may be SEQ ID NO: 4282 and the βCDR3 may be SEQ ID NO: 4296. The αCDR3 may be SEQ ID NO: 4283 and the βCDR3 may be SEQ ID NO: 4297.

The TCR specific for A*02:01_GVYDGEEHSV (SEQ ID NO: 4271) may comprise a TRAV, a TRAJ, a TRBV, optionally a TRBD, and a TRBJ amino acid sequence, optionally a TRAC sequence and optionally a TRBC sequence. Such TCR may comprise TRAV13-1, TRAJ11, TRBV6-3, and TRBJ2-1. Such TCR may comprise TRAV14DV4, TRAJ54, TRBV4-3, TRBD1, and TRBJ2-4.

The TCR specific for A*02:01_GVYDGEEHSV (SEQ ID NO: 4271) may comprise an alpha VJ sequence. The alpha VJ sequence may be any one of SEQ ID NOS 4311-4312.

The TCR specific for A*02:01_GVYDGEEHSV (SEQ ID NO: 4271) may comprise a beta V(D)J sequence. The beta V(D)J sequence may be any one of SEQ ID NOS 4326-4327.

In some embodiments, the alpha VJ sequence is SEQ ID NO: 4311 and the beta V(D)J sequence is SEQ ID NO: 4326. In some embodiments, the alpha VJ sequence is SEQ ID NO: 4312 and the beta V(D)J sequence is SEQ ID NO: 4327.

### Engineered Cells

Also provided are cells such as cells that contain an antigen receptor, e.g., that contains an extracellular domain including an anti-HLA-PEPTIDE ABP (e.g., a CAR or TCR), described herein. Also provided are populations of such cells, and compositions containing such cells. In some embodiments, compositions or populations are enriched for such cells, such as in which cells expressing the HLA-PEPTIDE ABP make up at least 1, 5, 10, 20, 30, 40, 50, 60, 70, 80, 90, 91, 92, 93, 94, 95, 96, 97, 98, 99, or more than 99 percent of the total cells in the composition or cells of a certain type such as T cells or CD8+ or CD4+ cells. In some embodiments, a composition comprises at least one cell containing an antigen receptor disclosed herein. Among the compositions are pharmaceutical compositions and formulations for administration, such as for adoptive cell therapy. Also provided are therapeutic methods for administering the cells and compositions to subjects, e.g., patients.

Thus also provided are genetically engineered cells expressing an ABP comprising a receptor, e.g., a TCR or CAR. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MALT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

The cells may be genetically modified to reduce expression or knock out endogenous TCRs. Such modifications are described in Mol Ther Nucleic Acids. 2012 Dec.; 1(12): e63; Blood. 2011 Aug. 11; 118(6):1495-503; Blood. 2012 Jun. 14; 119(24): 5697-5705; Torikai, Hiroki et al “HLA and TCR Knockout by Zinc Finger Nucleases: Toward “off-the-Shelf” Allogeneic T-Cell Therapy for CD19+ Malignancies.” Blood 116.21 (2010): 3766; Blood. 2018 Jan. 18; 131(3):311-322. doi: 10.1182/blood-2017-05-787598; and WO2016069283, which are incorporated by reference in their entirety.

The cells may be genetically modified to promote cytokine secretion. Such modifications are described in Hsu C, Hughes M S, Zheng Z, Bray R B, Rosenberg S A, Morgan R A. Primary human T lymphocytes engineered with a codon-optimized IL-15 gene resist cytokine withdrawal-induced apoptosis and persist long-term in the absence of exogenous cytokine. J Immunol. 2005; 175:7226-34; Quintarelli C, Vera J F, Savoldo B, Giordano Attianese G M, Pule M, Foster A E, Co-expression of cytokine and suicide genes to enhance the activity and safety of tumor-specific cytotoxic T lymphocytes. Blood. 2007; 110:2793-802; and Hsu C, Jones S A, Cohen C J, Zheng Z, Kerstann K, Zhou J, Cytokine-independent growth and clonal expansion of a primary human CD8+ T-cell clone following retroviral transduction with the IL-15 gene. Blood. 2007; 109:5168-77.

Mismatching of chemokine receptors on T cells and tumor-secreted chemokines has been shown to account for the suboptimal trafficking of T cells into the tumor microenvironment. To improve efficacy of therapy, the cells may be genetically modified to increase recognition of chemokines in tumor micro environment. Examples of such modifications are described in Moon, EKCarpenito, CSun, JWang, LCKapoor, VPredina, J Expression of a functional CCR2 receptor enhances tumor localization and tumor eradication by retargeted human T cells expressing a mesothelin-specific chimeric antibody receptor. Clin Cancer Res. 2011; 17: 4719-4730; and Craddock, JALu, ABear, APule, MBrenner, MKRooney, C M et al. Enhanced tumor trafficking of GD2 chimeric antigen receptor T cells by expression of the chemokine receptor CCR2b. J Immunother. 2010; 33: 780-788.

The cells may be genetically modified to enhance expression of costimulatory/enhancing receptors, such as CD28 and 41BB.

Adverse effects of T cell therapy can include cytokine release syndrome and prolonged B-cell depletion. Introduction of a suicide/safety switch in the recipient cells may improve the safety profile of a cell-based therapy. Accordingly, the cells may be genetically modified to include a suicide/safety switch. The suicide/safety switch may be a gene that confers sensitivity to an agent, e.g., a drug, upon the cell in which the gene is expressed, and which causes the cell to die when the cell is contacted with or exposed to the agent. Exemplary suicide/safety switches are described in Protein Cell. 2017 August; 8(8): 573-589. The suicide/safety switch may be HSV-TK. The suicide/safety switch may be cytosine daminase, purine nucleoside phosphorylase, or nitroreductase. The suicide/safety switch may be RapaCIDe™, described in U.S. Patent Application Pub. No. US20170166877A1. The suicide/safety switch system may be CD20/Rituximab, described in Haematologica. 2009 September; 94(9): 1316-1320. These references are incorporated by reference in their entirety.

The TCR or CAR may be introduced into the recipient cell as a split receptor which assembles only in the presence of a heterodimerizing small molecule. Such systems are described in Science. 2015 Oct. 16; 350(6258): aab4077, and in U.S. Pat. No. 9,587,020, which are hereby incorporated by reference.

In some embodiments, the cells include one or more nucleic acids, e.g., a polynucleotide encoding a TCR or CAR disclosed herein, wherein the polynucleotide is introduced via genetic engineering, and thereby express recombinant or genetically engineered TCRs or CARs as disclosed herein. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The nucleic acids may include a codon-optimized nucleotide sequence. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

A construct or vector may be used to introduce the TCR or CAR into the recipient cell. Exemplary constructs are described herein. Polynucleotides encoding the alpha and beta chains of the TCR or CAR may in a single construct or in separate constructs. The polynucleotides encoding the alpha and beta chains may be operably linked to a promoter, e.g., a heterologous promoter. The heterologous promoter may be a strong promoter, e.g., EF1alpha, CMV, PGK1, Ubc, beta actin, CAG promoter, and the like. The heterologous promoter may be a weak promoter. The heterologous promoter may be an inducible promoter. Exemplary inducible promoters include, but are not limited to TRE, NFAT, GAL4, LAC, and the like. Other exemplary inducible expression systems are described in U.S. Pat. Nos. 5,514,578; 6,245,531; 7,091,038 and European Patent No. 0517805, which are incorporated by reference in their entirety.

The construct for introducing the TCR or CAR into the recipient cell may also comprise a polynucleotide encoding a signal peptide (signal peptide element). The signal peptide may promote surface trafficking of the introduced TCR or CAR. Exemplary signal peptides include, but are not limited to CD8 signal peptide, immunoglobulin signal peptides, where specific examples include GM-CSF and IgG kappa. Such signal peptides are described in Trends Biochem Sci. 2006 October; 31(10):563-71. Epub 2006 Aug. 21; and An, et al. “Construction of a New Anti-CD19 Chimeric Antigen Receptor and the Anti-Leukemia Function Study of the Transduced T Cells.” Oncotarget 7.9 (2016): 10638-10649. PMC. Web. 16 Aug. 2018; which are hereby incorporated by reference.

In some cases, e.g., cases where the alpha and beta chains are expressed from a single construct or open reading frame, or cases wherein a marker gene is included in the construct, the construct may comprise a ribosomal skip sequence. The ribosomal skip sequence may be a 2A peptide, e.g., a P2A or T2A peptide. Exemplary P2A and T2A peptides are described in Scientific Reports volume 7, Article number: 2193 (2017), hereby incorporated by reference in its entirety. In some cases, a FURIN/PACE cleavage site is introduced upstream of the 2A element. FURIN/PACE cleavage sites are described in, e.g., http://www.nuolan.net/substrates.html. The cleavage peptide may also be a factor Xa cleavage site. In cases where the alpha and beta chains are expressed from a single construct or open reading frame, the construct may comprise an internal ribosome entry site (IRES).

The construct may further comprise one or more marker genes. Exemplary marker genes include but are not limited to GFP, luciferase, HA, lacZ. The marker may be a selectable marker, such as an antibiotic resistance marker, a heavy metal resistance marker, or a biocide resistant marker, as is known to those of skill in the art. The marker may be a complementation marker for use in an auxotrophic host. Exemplary complementation markers and auxotrophic hosts are described in Gene. 2001 Jan. 24; 263(1-2):159-69. Such markers may be expressed via an IRES, a frameshift sequence, a 2A peptide linker, a fusion with the TCR or CAR, or expressed separately from a separate promoter.

Exemplary vectors or systems for introducing TCRs or CARs into recipient cells include, but are not limited to Adeno-associated virus, Adenovirus, Adenovirus+Modified vaccinia, Ankara virus (MVA), Adenovirus+Retrovirus, Adenovirus+Sendai virus, Adenovirus+Vaccinia virus, Alphavirus (VEE) Replicon Vaccine, Antisense oligonucleotide, Bifidobacterium longum, CRISPR-Cas9, E. coli, Flavivirus, Gene gun, Herpesviruses, Herpes simplex virus, Lactococcus lactis, Electroporation, Lentivirus, Lipofection, Listeria monocytogenes, Measles virus, Modified Vaccinia Ankara virus (MVA), mRNA Electroporation, Naked/Plasmid DNA, Naked/Plasmid DNA+Adenovirus, Naked/Plasmid DNA+Modified Vaccinia Ankara virus (MVA), Naked/Plasmid DNA+RNA transfer, Naked/Plasmid DNA+Vaccinia virus, Naked/Plasmid DNA+Vesicular stomatitis virus, Newcastle disease virus, Non-viral, PiggyBac™ (PB) Transposon, nanoparticle-based systems, Poliovirus, Poxvirus, Poxvirus+Vaccinia virus, Retrovirus, RNA transfer, RNA transfer+Naked/Plasmid DNA, RNA virus, Saccharomyces cerevisiae, Salmonella typhimurium, Semliki forest virus, Sendai virus, Shigella dysenteriae, Simian virus, siRNA, Sleeping Beauty transposon, Streptococcus mutans, Vaccinia virus, Venezuelan equine encephalitis virus replicon, Vesicular stomatitis virus, and Vibrio cholera.

In preferred embodiments, the TCR or CAR is introduced into the recipient cell via adeno associated virus (AAV), adenovirus, CRISPR-CAS9, herpesvirus, lentivirus, lipofection, mRNA electroporation, PiggyBac™ (PB) Transposon, retrovirus, RNA transfer, or Sleeping Beauty transposon.

In some embodiments, a vector for introducing a TCR or CAR into a recipient cell is a viral vector. Exemplary viral vectors include adenoviral vectors, adeno-associated viral (AAV) vectors, lentiviral vectors, herpes viral vectors, retroviral vectors, and the like. Such vectors are described herein.

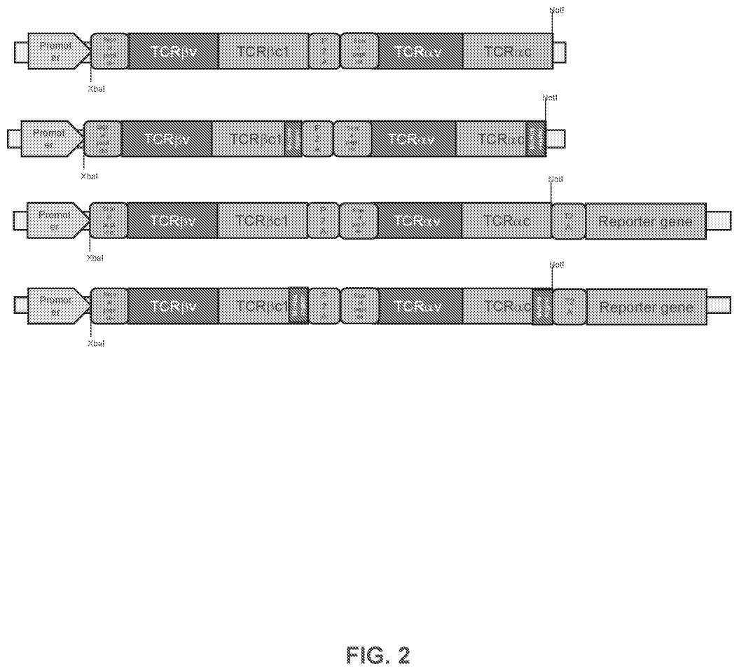

Exemplary embodiments of TCR constructs for introducing a TCR or CAR into recipient cells is shown in FIG. 2. In some embodiments, a TCR construct includes, from the 5′-3′ direction, the following polynucleotide sequences: a promoter sequence, a signal peptide sequence, a TCR β variable (TCRPβv) sequence, a TCR β constant ((TCRβc) sequence, a cleavage peptide (e.g., P2A), a signal peptide sequence, a TCR α variable (TCRαv) sequence, and a TCR α constant (TCRαc) sequence. In some embodiments, the TCRβc and TCRαc sequences of the construct include one or more murine regions, e.g., full murine constant sequences or human [symbol] murine amino acid exchanges as described herein. In some embodiments, the construct further includes, 3′ of the TCRαc sequence, a cleavage peptide sequence (e.g., T2A) followed by a reporter gene. In an embodiment, the construct includes, from the 5′-3′ direction, the following polynucleotide sequences: a promoter sequence, a signal peptide sequence, a TCR β variable (TCRβv) sequence, a TCR β constant ((TCRβc) sequence containing one or more murine regions, a cleavage peptide (e.g., P2A), a signal peptide sequence, a TCR α variable (TCRαv) sequence, and a TCR α constant (TCRαc) sequence containing one or more murine regions, a cleavage peptide (e.g., T2A), and a reporter gene.

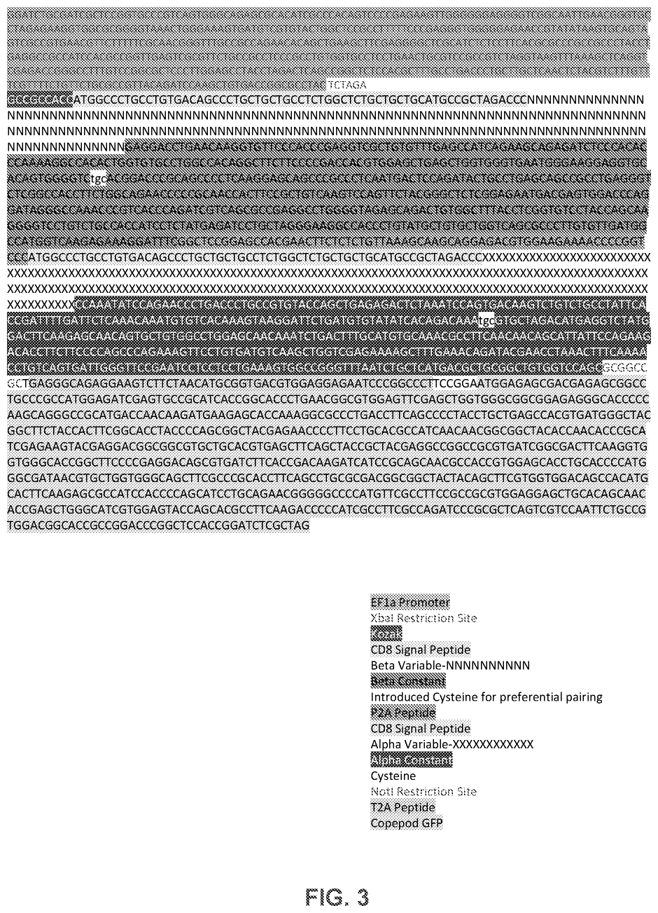

FIG. 3 depicts an exemplary construct backbone sequence for cloning TCRs into expression systems for therapy development.

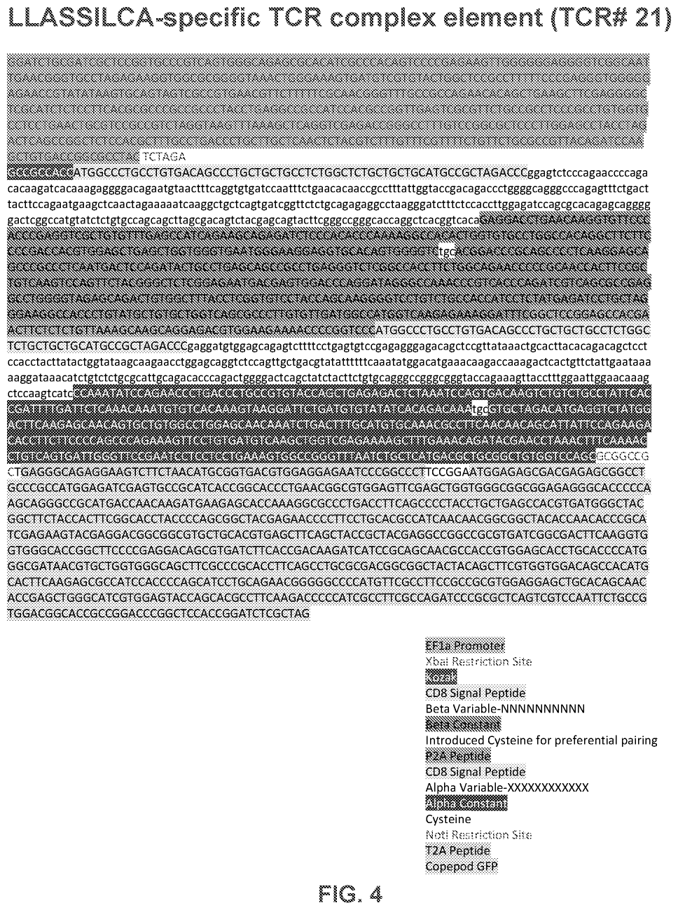

FIG. 4 depicts an exemplary construct sequence for cloning an identified A*0201_LLASSILCA-specific TCR into expression systems for therapy development.

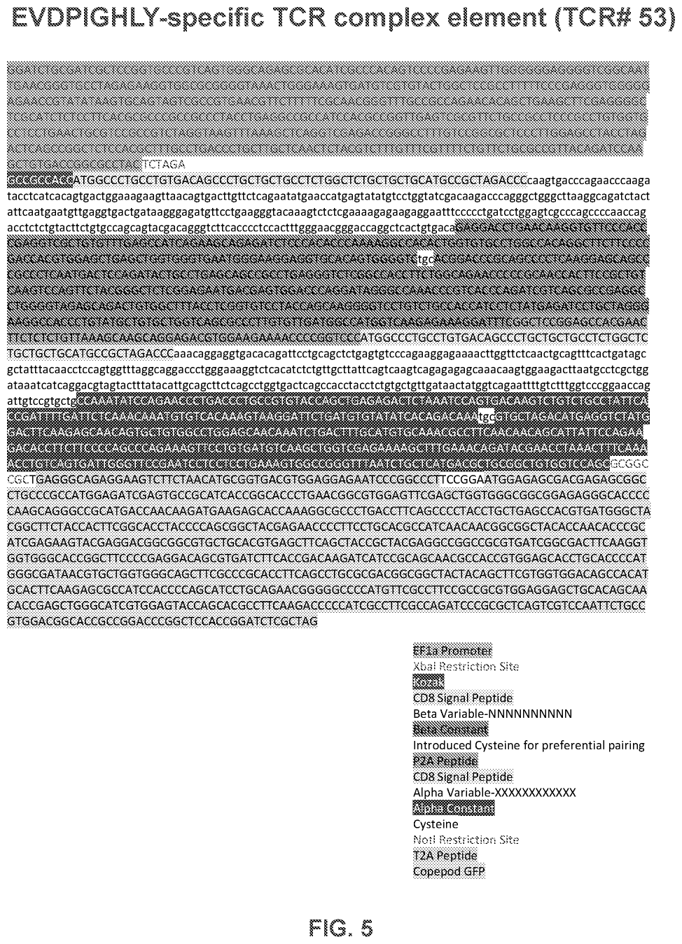

FIG. 5 depicts an exemplary construct sequence for cloning an identified A*0101_EVDPIGHLY-specific TCR into expression systems for therapy development.

Nucleotides, Vectors, Host Cells, and Related Methods

Also provided are isolated nucleic acids encoding HLA-PEPTIDE ABPs, vectors comprising the nucleic acids, and host cells comprising the vectors and nucleic acids, as well as recombinant techniques for the production of the ABPs.

The nucleic acids may be recombinant. The recombinant nucleic acids may be constructed outside living cells by joining natural or synthetic nucleic acid segments to nucleic acid molecules that can replicate in a living cell, or replication products thereof. For purposes herein, the replication can be in vitro replication or in vivo replication.

For recombinant production of an ABP, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include one or more of the following: a signal sequence, an origin of replication, one or more marker genes, an enhancer element, a promoter, and a transcription termination sequence, for example as described in U.S. Pat. No. 5,534,615, incorporated by reference in its entirety.

Exemplary vectors or constructs suitable for expressing an ABP, e.g., a TCR, CAR, antibody, or antigen binding fragment thereof, include, e.g., the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as AGT1O, AGTl 1, AZapII (Stratagene), AEMBL4, and ANMl 149, are also suitable for expressing an ABP disclosed herein.

Illustrative examples of suitable host cells are provided below. These host cells are not meant to be limiting, and any suitable host cell may be used to produce the ABPs provided herein.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P aeruginosa), and Streptomyces. One useful E. coli cloning host is E. coli 294, although other strains such as E. coli B, E. coli X1776, and E. coli W3110 are also suitable.

In addition to prokaryotes, eukaryotic microbes such as filamentous fungi or yeast are also suitable cloning or expression hosts for HLA-PEPTIDE ABP-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the HLA-PEPTIDE ABP may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469; or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the ABP can be produced intracellularly, in the periplasmic space, or directly secreted into the medium. If the ABP is produced intracellularly, as a first step, the particulate debris, either host cells or lysed fragments, is removed, for example, by centrifugation or ultrafiltration. For example, Carter et al. (Bio/Technology, 1992, 10:163-167, incorporated by reference in its entirety) describes a procedure for isolating ABPs which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the ABP is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the ABP may be useful, for example, where the ABP accumulates in a cell as an insoluble aggregate, or where yields from periplasmic expression are low.

Where the ABP is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The ABP composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the ABP Protein A can be used to purify ABPs that comprise human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the ABP comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the ABP of interest and contaminants may be subjected to low pH hydrophobic interaction chromatography using an elution buffer at a pH between about 2.5 to about 4.5, generally performed at low salt concentrations (e.g., from about 0 to about 0.25 M salt).

Methods of Making HLA-PEPTIDE ABPs

HLA-PEPTIDE Antigen Preparation

The HLA-PEPTIDE antigen used for isolation or creation of the ABPs provided herein may be intact HLA-PEPTIDE or a fragment of HLA-PEPTIDE. The HLA-PEPTIDE antigen may be, for example, in the form of isolated protein or a protein expressed on the surface of a cell.

In some embodiments, the HLA-PEPTIDE antigen is a non-naturally occurring variant of HLA-PEPTIDE, such as a HLA-PEPTIDE protein having an amino acid sequence or post-translational modification that does not occur in nature.

In some embodiments, the HLA-PEPTIDE antigen is truncated by removal of, for example, intracellular or membrane-spanning sequences, or signal sequences. In some embodiments, the HLA-PEPTIDE antigen is fused at its C-terminus to a human IgG1 Fc domain or a polyhistidine tag.

### Methods of Identifying ABPs

ABPs that bind HLA-PEPTIDE can be identified using any method known in the art, e.g., phage display or immunization of a subject.

One method of identifying an antigen binding protein includes providing at least one HLA-PEPTIDE target; and binding the at least one target with an antigen binding protein, thereby identifying the antigen binding protein. The antigen binding protein can be present in a library comprising a plurality of distinct antigen binding proteins.

In some embodiments, the library is a phage display library. The phage display library can be developed so that it is substantially free of antigen binding proteins that non-specifically bind the HLA of the HLA-PEPTIDE target. The antigen binding protein can be present in a yeast display library comprising a plurality of distinct antigen binding proteins. The yeast display library can be developed so that it is substantially free of antigen binding proteins that non-specifically bind the HLA of the HLA-PEPTIDE target.

In some embodiments, the library is a yeast display library.

In some embodiments, the library is a TCR display library. Exemplary TCR display libraries and methods of using such TCR display libraries are described in WO 98/39482; WO 01/62908; WO 2004/044004; WO2005116646, WO201401668863 WO2015136072, WO2017046198; and Helmut et al, (2000) PNAS 97 (26) 14578-14583, which are hereby incorporated by reference in their entirety.

In some aspects, the binding step is performed more than once, optionally at least three times, e.g., at least 1, 2, 3, 4, 5, 6, 7, 8, 9, or 10×.

In addition, the method can also include contacting the antigen binding protein with one or more peptide-HLA complexes that are distinct from the HLA-PEPTIDE target to determine if the antigen binding protein selectively binds the HLA-PEPTIDE target.

Another method of identifying an antigen binding protein can include obtaining at least one HLA-PEPTIDE target; administering the HLA-PEPTIDE target to a subject (e.g., a mouse, rabbit or a llama), optionally in combination with an adjuvant; and isolating the antigen binding protein from the subject. Isolating the antigen binding protein can include screening the serum of the subject to identify the antigen binding protein. The method can also include contacting the antigen binding protein with one or more peptide-HLA complexes that are distinct from the HLA-PEPTIDE target, e.g., to determine if the antigen binding protein selectively binds to the HLA-PEPTIDE target. An antigen binding protein that is identified can be humanized.

In some aspects, isolating the antigen binding protein comprises isolating a B cell from the subject that expresses the antigen binding protein. The B cell can be used to create a hybridoma. The B cell can also be used for cloning one or more of its CDRs. The B cell can also be immortalized, for example, by using EBV transformation. Sequences encoding an antigen binding protein can be cloned from immortalized B cells or can be cloned directly from B cells isolated from an immunized subject. A library that comprises the antigen binding protein of the B cell can also be created, optionally wherein the library is phage display or yeast display.

Another method of identifying an antigen binding protein can include obtaining a cell comprising the antigen binding protein; contacting the cell with an HLA-multimer (e.g., a tetramer) comprising at least one HLA-PEPTIDE target; and identifying the antigen binding protein via binding between the HLA-multimer and the antigen binding protein.

The cell can be, e.g., a T cell, optionally a CTL, or an NK cell, for example. The method can further include isolating the cell, optionally using flow cytometry, magnetic separation, or single cell separation. The method can further include sequencing the antigen binding protein.

Another method of identifying an antigen binding protein can include obtaining one or more cells comprising the antigen binding protein; activating the one or more cells with at least one HLA-PEPTIDE target presented on at least one antigen presenting cell (APC); and identifying the antigen binding protein via selection of one or more cells activated by interaction with at least one HLA-PEPTIDE target.

The cell can be, e.g., a T cell, optionally a CTL, or an NK cell, for example. The method can further include isolating the cell, optionally using flow cytometry, magnetic separation, or single cell separation. The method can further include sequencing the antigen binding protein.

### Methods of Making Monoclonal ABPs

Monoclonal ABPs may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal ABPs may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing ABPs that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal ABPs: Principles and Practice 3^{rd} ed. (1986) Academic Press, San Diego, Calif., incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of ABP by the selected ABP-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, Calif.), and SP-2 or X63-Ag8-653 cells (available from the American Type Culture Collection, Rockville, Md.). Human myeloma and mouse-human heteromyeloma cell lines also have been described for the production of human monoclonal ABPs. See e.g., Kozbor, J. Immunol., 1984, 133:3001, incorporated by reference in its entirety.

After the identification of hybridoma cells that produce ABPs of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal ABPs may be readily isolated and sequenced using conventional procedures (e.g., by using oligonucleotide probes that are capable of binding specifically to genes encoding the heavy and light chains of the monoclonal ABPs). Thus, the hybridoma cells can serve as a useful source of DNA encoding ABPs with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce ABP, to produce the monoclonal ABPs.

### Methods of Making Chimeric ABPs

Illustrative methods of making chimeric ABPs are described, for example, in U.S. Pat. No. 4,816,567; and Morrison et al., Proc. Natl. Acad. Sci. USA, 1984, 81:6851-6855; each of which is incorporated by reference in its entirety. In some embodiments, a chimeric ABP is made by using recombinant techniques to combine a non-human variable region (e.g., a variable region derived from a mouse, rat, hamster, rabbit, or non-human primate, such as a monkey) with a human constant region.

### Methods of Making Humanized ABPs

Humanized ABPs may be generated by replacing most, or all, of the structural portions of a non-human monoclonal ABP with corresponding human ABP sequences. Consequently, a hybrid molecule is generated in which only the antigen-specific variable, or CDR, is composed of non-human sequence. Methods to obtain humanized ABPs include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

### Methods of Making Human ABPs

Human ABPs can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human ABPs can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human ABPs may also be generated by in vitro activated B cells (see e.g., U.S. Pat. Nos. 5,567,610 and 5,229,275, each of which is incorporated by reference in its entirety). Human ABPs may also be derived from yeast-based libraries (see e.g., U.S. Pat. No. 8,691,730, incorporated by reference in its entirety).

### Methods of Making ABP Fragments

The ABP fragments provided herein may be made by any suitable method, including the illustrative methods described herein or those known in the art. Suitable methods include recombinant techniques and proteolytic digestion of whole ABPs. Illustrative methods of making ABP fragments are described, for example, in Hudson et al., Nat. Med., 2003, 9:129-134, incorporated by reference in its entirety. Methods of making scFv ABPs are described, for example, in Pluckthun, in The Pharmacology of Monoclonal ABPs, vol. 113, Rosenburg and Moore eds., Springer-Verlag, New York, pp. 269-315 (1994); WO 93/16185; and U.S. Pat. Nos. 5,571,894 and 5,587,458; each of which is incorporated by reference in its entirety.

### Methods of Making Alternative Scaffolds

The alternative scaffolds provided herein may be made by any suitable method, including the illustrative methods described herein or those known in the art. For example, methods of preparing Adnectins™ are described in Emanuel et al., mAbs, 2011, 3:38-48, incorporated by reference in its entirety. Methods of preparing iMabs are described in U.S. Pat. Pub. No. 2003/0215914, incorporated by reference in its entirety. Methods of preparing Anticalins® are described in Vogt and Skerra, Chem. Biochem., 2004, 5:191-199, incorporated by reference in its entirety. Methods of preparing Kunitz domains are described in Wagner et al., Biochem. & Biophys. Res. Comm., 1992, 186:118-1145, incorporated by reference in its entirety. Methods of preparing thioredoxin peptide aptamers are provided in Geyer and Brent, Meth. Enzymol., 2000, 328:171-208, incorporated by reference in its entirety. Methods of preparing Affibodies are provided in Fernandez, Curr. Opinion in Biotech., 2004, 15:364-373, incorporated by reference in its entirety. Methods of preparing DARPins are provided in Zahnd et al., J. Mol. Biol., 2007, 369:1015-1028, incorporated by reference in its entirety. Methods of preparing Affilins are provided in Ebersbach et al., J. Mol. Biol., 2007, 372:172-185, incorporated by reference in its entirety. Methods of preparing Tetranectins are provided in Graversen et al., J. Biol. Chem., 2000, 275:37390-37396, incorporated by reference in its entirety. Methods of preparing Avimers are provided in Silverman et al., Nature Biotech., 2005, 23:1556-1561, incorporated by reference in its entirety. Methods of preparing Fynomers are provided in Silacci et al., J. Biol. Chem., 2014, 289:14392-14398, incorporated by reference in its entirety. Further information on alternative scaffolds is provided in Binz et al., Nat. Biotechnol., 2005 23:1257-1268; and Skerra, Current Opin. in Biotech., 2007 18:295-304, each of which is incorporated by reference in its entirety.

### Methods of Making Multispecific ABPs

The multispecific ABPs provided herein may be made by any suitable method, including the illustrative methods described herein or those known in the art. Methods of making common light chain ABPs are described in Merchant et al., Nature Biotechnol., 1998, 16:677-681, incorporated by reference in its entirety. Methods of making tetravalent bispecific ABPs are described in Coloma and Morrison, Nature Biotechnol., 1997, 15:159-163, incorporated by reference in its entirety. Methods of making hybrid immunoglobulins are described in Milstein and Cuello, Nature, 1983, 305:537-540; and Staerz and Bevan, Proc. Natl. Acad. Sci. USA, 1986, 83:1453-1457; each of which is incorporated by reference in its entirety. Methods of making immunoglobulins with knobs-into-holes modification are described in U.S. Pat. No. 5,731,168, incorporated by reference in its entirety. Methods of making immunoglobulins with electrostatic modifications are provided in WO 2009/089004, incorporated by reference in its entirety. Methods of making bispecific single chain ABPs are described in Traunecker et al., EMBO J., 1991, 10:3655-3659; and Gruber et al., J. Immunol., 1994, 152:5368-5374; each of which is incorporated by reference in its entirety. Methods of making single-chain ABPs, whose linker length may be varied, are described in U.S. Pat. Nos. 4,946,778 and 5,132,405, each of which is incorporated by reference in its entirety. Methods of making diabodies are described in Hollinger et al., Proc. Natl. Acad. Sci. USA, 1993, 90:6444-6448, incorporated by reference in its entirety. Methods of making triabodies and tetrabodies are described in Todorovska et al., J. Immunol. Methods, 2001, 248:47-66, incorporated by reference in its entirety. Methods of making trispecific F(ab′)3 derivatives are described in Tutt et al. J. Immunol., 1991, 147:60-69, incorporated by reference in its entirety. Methods of making cross-linked ABPs are described in U.S. Pat. No. 4,676,980; Brennan et al., Science, 1985, 229:81-83; Staerz, et al. Nature, 1985, 314:628-631; and EP 0453082; each of which is incorporated by reference in its entirety. Methods of making antigen-binding domains assembled by leucine zippers are described in Kostelny et al., J. Immunol., 1992, 148:1547-1553, incorporated by reference in its entirety. Methods of making ABPs via the DNL approach are described in U.S. Pat. Nos. 7,521,056; 7,550,143; 7,534,866; and 7,527,787; each of which is incorporated by reference in its entirety. Methods of making hybrids of ABP and non-ABP molecules are described in WO 93/08829, incorporated by reference in its entirety, for examples of such ABPs. Methods of making DAF ABPs are described in U.S. Pat. Pub. No. 2008/0069820, incorporated by reference in its entirety. Methods of making ABPs via reduction and oxidation are described in Carlring et al., PLoS One, 2011, 6:e22533, incorporated by reference in its entirety. Methods of making DVD-Igs™ are described in U.S. Pat. No. 7,612,181, incorporated by reference in its entirety. Methods of making DARTs™ are described in Moore et al., Blood, 2011, 117:454-451, incorporated by reference in its entirety. Methods of making DuoBodies® are described in Labrijn et al., Proc. Natl. Acad. Sci. USA, 2013, 110:5145-5150; Gramer et al., mAbs, 2013, 5:962-972; and Labrijn et al., Nature Protocols, 2014, 9:2450-2463; each of which is incorporated by reference in its entirety. Methods of making ABPs comprising scFvs fused to the C-terminus of the C_{H3} from an IgG are described in Coloma and Morrison, Nature Biotechnol., 1997, 15:159-163, incorporated by reference in its entirety. Methods of making ABPs in which a Fab molecule is attached to the constant region of an immunoglobulin are described in Miler et al., J. Immunol., 2003, 170:4854-4861, incorporated by reference in its entirety. Methods of making CovX-Bodies are described in Doppalapudi et al., Proc. Natl. Acad. Sci. USA, 2010, 107:22611-22616, incorporated by reference in its entirety. Methods of making Fcab ABPs are described in Wozniak-Knopp et al., Protein Eng. Des. Sel., 2010, 23:289-297, incorporated by reference in its entirety. Methods of making TandAb® ABPs are described in Kipriyanov et al., J. Mol. Biol., 1999, 293:41-56 and Zhukovsky et al., Blood, 2013, 122:5116, each of which is incorporated by reference in its entirety. Methods of making tandem Fabs are described in WO 2015/103072, incorporated by reference in its entirety. Methods of making Zybodies™ are described in LaFleur et al., mAbs, 2013, 5:208-218, incorporated by reference in its entirety.

### Methods of Making Variants

Any suitable method can be used to introduce variability into a polynucleotide sequence(s) encoding an ABP, including error-prone PCR, chain shuffling, and oligonucleotide-directed mutagenesis such as trinucleotide-directed mutagenesis (TRIM). In some aspects, several CDR residues (e.g., 4-6 residues at a time) are randomized. CDR residues involved in antigen binding may be specifically identified, for example, using alanine scanning mutagenesis or modeling. CDR-H3 and CDR-L3 in particular are often targeted for mutation.

The introduction of diversity into the variable regions and/or CDRs can be used to produce a secondary library. The secondary library is then screened to identify ABP variants with improved affinity. Affinity maturation by constructing and reselecting from secondary libraries has been described, for example, in Hoogenboom et al., Methods in Molecular Biology, 2001, 178:1-37, incorporated by reference in its entirety.

Methods for Engineering Cells with ABPs

Also provided are methods, nucleic acids, compositions, and kits, for expressing the ABPs, including receptors comprising antibodies, CARs, and TCRs, and for producing genetically engineered cells expressing such ABPs. The genetic engineering generally involves introduction of a nucleic acid encoding the recombinant or engineered component into the cell, such as by retroviral transduction, transfection, or transformation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II: 223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines or other factors. Various methods for the introduction of genetically engineered components, e.g., antigen receptors, e.g., CARs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298; Van Tedeloo et al. (2000) Gene Therapy 7(16): 1431-1437; and Roth et al. (2018) Nature 559:405-409). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

Preparation of Engineered Cells

In some embodiments, preparation of the engineered cells includes one or more culture and/or preparation steps. The cells for introduction of the HLA-PEPTIDE-ABP, e.g., TCR or CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using CD3/CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining TCM-enriched CD8+ T cells and CD4+ T cells further enhances efficacy.

In embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or ROR1, and positive selection based on a marker characteristic of central memory T cells, such as CD62L or CCR7, where the positive and negative selections are carried out in either order.

CD4+T helper cells are sorted into naive, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L- and CD45RO−

In one example, to enrich for CD4+ cells by negative selection, a monoclonal antibody cocktail typically includes antibodies to CD14, CD20, CD1 b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immune-magnetic (or affinity-magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotic), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This can then be diluted 1:1 with media so that the final concentration of DMSO and HSA are 10% and 4%, respectively. Other examples include Cryostor®, CTL-Cryo™ ABC freezing media, and the like. The cells are then frozen to −80 degrees C. at a rate of 1 degree per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some embodiments, the PBMC feeder cells are inactivated with Mytomicin C. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

Assays

A variety of assays known in the art may be used to identify and characterize an HLA-PEPTIDE ABP provided herein.

### Binding, Competition, and Epitope Mapping Assays

Specific antigen-binding activity of an ABP provided herein may be evaluated by any suitable method, including using SPR, BLI, RIA and MSD-SET, as described elsewhere in this disclosure. Additionally, antigen-binding activity may be evaluated by ELISA assays, using flow cytometry, and/or Western blot assays.

Assays for measuring competition between two ABPs, or an ABP and another molecule (e.g., one or more ligands of HLA-PEPTIDE such as a TCR) are described elsewhere in this disclosure and, for example, in Harlow and Lane, ABPs: A Laboratory Manual ch. 14, 1988, Cold Spring Harbor Laboratory, Cold Spring Harbor, N.Y, incorporated by reference in its entirety.

Assays for mapping the epitopes to which an ABP provided herein bind are described, for example, in Morris “Epitope Mapping Protocols,” in Methods in Molecular Biology vol. 66, 1996, Humana Press, Totowa, N.J., incorporated by reference in its entirety. In some embodiments, the epitope is determined by peptide competition. In some embodiments, the epitope is determined by mass spectrometry. In some embodiments, the epitope is determined by mutagenesis. In some embodiments, the epitope is determined by crystallography.

### Assays for Effector Functions

Effector function following treatment with an ABP and/or cell provided herein may be evaluated using a variety of in vitro and in vivo assays known in the art, including those described in Ravetch and Kinet, Annu. Rev Immunol., 1991, 9:457-492; U.S. Pat. Nos. 5,500,362, 5,821,337; Hellstrom et al., Proc. Nat'l Acad. Sci. USA, 1986, 83:7059-7063; Hellstrom et al., Proc. Nat'l Acad. Sci. USA, 1985, 82:1499-1502; Bruggemann et al., J. Exp. Med., 1987, 166:1351-1361; Clynes et al., Proc. Nat'l Acad. Sci. USA, 1998, 95:652-656; WO 2006/029879; WO 2005/100402; Gazzano-Santoro et al., J. Immunol. Methods, 1996, 202:163-171; Cragg et al., Blood, 2003, 101:1045-1052; Cragg et al. Blood, 2004, 103:2738-2743; and Petkova et al., Int'l. Immunol., 2006, 18:1759-1769; each of which is incorporated by reference in its entirety.

Pharmaceutical Compositions

An ABP, cell, or HLA-PEPTIDE target provided herein can be formulated in any appropriate pharmaceutical composition and administered by any suitable route of administration. Suitable routes of administration include, but are not limited to, the intra-arterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethyl siloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include ethanol, poly(ethylene) glycol, butylene glycol, dimethylacetamide, glycerin, propylene glycol, and combinations thereof.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, monosodium glutamate, and combinations thereof.

In some embodiments, the pharmaceutical composition comprises a carrier or filler. Illustrative examples of carriers or fillers include lactose, maltodextrin, mannitol, sorbitol, chitosan, stearic acid, xanthan gum, guar gum, and combinations thereof.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, vitamin E polyethylene(glycol) succinate, and combinations thereof.

In some embodiments, the pharmaceutical composition comprises an anti-caking agent. Illustrative examples of anti-caking agents include calcium phosphate (tribasic), hydroxymethyl cellulose, hydroxypropyl cellulose, magnesium oxide, and combinations thereof.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, sugars, and combinations thereof. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an ABP, since water can facilitate the degradation of some ABPs.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

In certain embodiments, an ABP and/or cell provided herein is formulated as parenteral dosage forms. Parenteral dosage forms can be administered to subjects by various routes including, but not limited to, subcutaneous, intravenous (including infusions and bolus injections), intramuscular, and intra-arterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry (e.g., lyophilized) products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the ABPs and/or cells disclosed herein can also be incorporated into the parenteral dosage forms.

In some embodiments, the parenteral dosage form is lyophilized. Exemplary lyophilized formulations are described, for example, in U.S. Pat. Nos. 6,267,958 and 6,171,586; and WO 2006/044908; each of which is incorporated by reference in its entirety.

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic ABP.

The amount of the ABP, cell, or composition which will be effective in the prevention or treatment of a disorder or one or more symptoms thereof will vary with the nature and severity of the disease or condition, and the route by which the ABP and/or cell is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the ABPs and/or cells provided herein are also encompassed by the dosage amounts and dose frequency schedules provided herein. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an ABP or composition provided herein followed by one or more maintenance doses.

In certain embodiments, a dose of an ABP, cell, or composition provided herein can be administered to achieve a steady-state concentration of the ABP and/or cell in blood or serum of the subject. The steady-state concentration can be determined by measurement according to techniques available to those of skill or can be based on the physical characteristics of the subject such as height, weight and age.

As discussed in more detail elsewhere in this disclosure, an ABP and/or cell provided herein may optionally be administered with one or more additional agents useful to prevent or treat a disease or disorder. The effective amount of such additional agents may depend on the amount of ABP present in the formulation, the type of disorder or treatment, and the other factors known in the art or described herein.

Therapeutic Applications

For therapeutic applications, ABPs and/or cells are administered to a mammal, generally a human, in a pharmaceutically acceptable dosage form such as those known in the art and those discussed above. For example, ABPs and/or cells may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The ABPs also are suitably administered by peritumoral, intralesional, or perilesional routes, to exert local as well as systemic therapeutic effects. The intraperitoneal route may be particularly useful, for example, in the treatment of ovarian tumors.

The ABPs and/or cells provided herein can be useful for the treatment of any disease or condition involving HLA-PEPTIDE. In some embodiments, the disease or condition is a disease or condition that can benefit from treatment with an anti-HLA-PEPTIDE ABP and/or cell. In some embodiments, the disease or condition is a tumor. In some embodiments, the disease or condition is a cell proliferative disorder. In some embodiments, the disease or condition is a cancer.

In some embodiments, the ABPs and/or cells provided herein are provided for use as a medicament. In some embodiments, the ABPs and/or cells provided herein are provided for use in the manufacture or preparation of a medicament. In some embodiments, the medicament is for the treatment of a disease or condition that can benefit from an anti-HLA-PEPTIDE ABP and/or cell. In some embodiments, the disease or condition is a tumor. In some embodiments, the disease or condition is a cell proliferative disorder. In some embodiments, the disease or condition is a cancer.

In some embodiments, provided herein is a method of treating a disease or condition in a subject in need thereof by administering an effective amount of an ABP and/or cell provided herein to the subject. In some aspects, the disease or condition is a cancer.

In some embodiments, provided herein is a method of treating a disease or condition in a subject in need thereof by administering an effective amount of an ABP and/or cell provided herein to the subject, wherein the disease or condition is a cancer, and the cancer is selected from a solid tumor and a hematological tumor.

In some embodiments, provided herein is a method of modulating an immune response in a subject in need thereof, comprising administering to the subject an effective amount of an ABP and/or cell or a pharmaceutical composition disclosed herein.

Combination Therapies

In some embodiments, an ABP and/or cell provided herein is administered with at least one additional therapeutic agent. Any suitable additional therapeutic agent may be administered with an ABP and/or cell provided herein. An additional therapeutic agent can be fused to an ABP. In some aspects, the additional therapeutic agent is selected from radiation, a cytotoxic agent, a toxin, a chemotherapeutic agent, a cytostatic agent, an anti-hormonal agent, an EGFR inhibitor, an immunomodulatory agent, an anti-angiogenic agent, and combinations thereof. In some embodiments, the additional therapeutic agent is an ABP.

Diagnostic Methods

Also provided are methods for predicting and/or detecting the presence of a given HLA-PEPTIDE on a cell from a subject. Such methods may be used, for example, to predict and evaluate responsiveness to treatment with an ABP and/or cell provided herein.

In some embodiments, a blood or tumor sample is obtained from a subject and the fraction of cells expressing HLA-PEPTIDE is determined. In some aspects, the relative amount of HLA-PEPTIDE expressed by such cells is determined. The fraction of cells expressing HLA-PEPTIDE and the relative amount of HLA-PEPTIDE expressed by such cells can be determined by any suitable method. In some embodiments, flow cytometry is used to make such measurements. In some embodiments, fluorescence assisted cell sorting (FACS) is used to make such measurement. See Li et al., J. Autoimmunity, 2003, 21:83-92 for methods of evaluating expression of HLA-PEPTIDE in peripheral blood.

In some embodiments, detecting the presence of a given HLA-PEPTIDE on a cell from a subject is performed using immunoprecipitation and mass spectrometry. This can be performed by obtaining a tumor sample (e.g., a frozen tumor sample) such as a primary tumor specimen and applying immunoprecipitation to isolate one or more peptides. The HLA alleles of the tumor sample can be determined experimentally or obtained from a third party source. The one or more peptides can be subjected to MS to determine their sequence(s). The spectra from the MS can then be searched against a database. An example is provided in the Examples section below.

In some embodiments, predicting the presence of a given HLA-PEPTIDE on a cell from a subject is performed using a computer-based model applied to the peptide sequence and/or RNA measurements of one or more genes comprising that peptide sequence (e.g., RNA seq or RT-PCR, or nanostring) from a tumor sample. The model used can be as described in international patent application no. PCT/US2016/067159, herein incorporated by reference, in its entirety, for all purposes.

Kits

Also provided are kits comprising an ABP and/or cell provided herein. The kits may be used for the treatment, prevention, and/or diagnosis of a disease or disorder, as described herein.

In some embodiments, the kit comprises a container and a label or package insert on or associated with the container. Suitable containers include, for example, bottles, vials, syringes, and IV solution bags. The containers may be formed from a variety of materials, such as glass or plastic. The container holds a composition that is by itself, or when combined with another composition, effective for treating, preventing and/or diagnosing a disease or disorder. The container may have a sterile access port. For example, if the container is an intravenous solution bag or a vial, it may have a port that can be pierced by a needle. At least one active agent in the composition is an ABP provided herein. The label or package insert indicates that the composition is used for treating the selected condition.

In some embodiments, the kit comprises (a) a first container with a first composition contained therein, wherein the first composition comprises an ABP and/or cell provided herein; and (b) a second container with a second composition contained therein, wherein the second composition comprises a further therapeutic agent. The kit in this embodiment can further comprise a package insert indicating that the compositions can be used to treat a particular condition, e.g., cancer.

Alternatively, or additionally, the kit may further comprise a second (or third) container comprising a pharmaceutically-acceptable excipient. In some aspects, the excipient is a buffer. The kit may further include other materials desirable from a commercial and user standpoint, including filters, needles, and syringes.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

### Example 1: Identification of Predicted HLA-PEPTIDE Complexes

We identified cancer specific HLA-peptide targets using three computational steps: First, we identified genes that are not generally expressed in most normal tissues using data available through the Genotype-Tissue Expression (GTEx) Project [1]. We then identified which of those genes are aberrantly expressed in cancer samples using data from The Cancer Genome Atlas (TCGA) Research Network: http://cancergenome.nih.gov/. In these genes, we identified which peptides are likely to be presented as cell surface antigens by MHC Class I proteins using a deep learning model trained on HLA presented peptides sequenced by MS/MS, as described in international patent application no. PCT/US2016/067159, herein incorporated by reference, in its entirety, for all purposes.

To identify genes that are not usually expressed in normal tissues, we obtained aggregated gene expression data from the Genotype-Tissue Expression (GTEx) Project (version V6p). This dataset comprised 8,555 post-mortem samples from over 50 tissue types. Expression was measured using RNA-Seq and computationally processed according to the GTEx standard pipeline (https://www.gtexportal.org/home/documentationPage). For the purposes of this analysis, genes were considered not expressed in normal tissues if they were found not to be expressed in any tissues in GTEx or were only expressed in one or more of testis, minor salivary gland, and the endocervix (i.e., immune privileged or non-essential tissues). We also restricted our search to only include protein coding genes. Because GTEx and TCGA use different annotations of the human genome in their computational analyses, we excluded genes which we could not map between the two datasets using standard techniques such as ENCODE mappings.

We sought to define criteria to excluded genes that were expressed in normal tissue that was strict to ensure tumor specificity, but would not exclude non-zero measurements arising from sporadic, low level transcription or potential artifacts such as read misalignment. Therefore, we designated a gene to be not normally expressed in a non-immune privileged or essential tissue if its median expression across GTEx samples was less than 0.5 RPKM (Reads Per Kilobase of transcript per Million mapped reads), and it was never expressed with greater than 10 RPKM, and it was expressed at 5 RPKM in no more than two samples across all essential tissue samples. To exclude genes which were potentially expressed but could not be measured by RNA-Seq using the GTEX analysis pipeline, we also excluded genes which were measured at 0 RPKM in all samples. These criteria left us with a set of protein coding genes that did not appear to be expressed in most normal tissues.

We next sought to identify which of these genes are aberrantly expressed in tumors. We examined 11,093 samples available from TCGA (Data Release 6.0). We considered a gene expressed if it was observed at expression of at least 5 FPKM (Fragments Per Kilobase of transcript per Million mapped reads) in at least 5 samples. Because one fragment usually consists of two mapped reads, 5 FPKM equals approximately 10 RPKM.

While the GTEx data spans a broad range of tissue types, it does not include all cell types that are present in the human body. We therefore further examined the list for the gene's biological function category using the DAVID v 6.8 [2] and used this analysis, along with literature review, to filter the gene list further. We removed genes likely to be expressed in immune cells (e.g., interferon family genes), eye-related genes (e.g., retina in the FANTOM5 dataset http://www.proteinatlas.org), genes expressed in the mouth and nose (e.g. olfactory genes and taste receptors), and genes related to the circadian cycle. We also excluded genes that are part of large gene families, including histone genes, because their expression is difficult to accurately assess with RNA Sequencing due to sequence homology.

We then examined the distribution of the expression of the remaining genes across the TCGA samples. When we examined the known Cancer Testis Antigens (CTAs), e.g., the MAGE family of genes, we observed that the expression of these genes in log space was generally characterized by a bimodal distribution across samples in the TCGA. This distribution included a left mode around a lower expression value and a right mode (or thick tail) at a higher expression level. This expression pattern is consistent with a biological model in which some minimal expression is detected at baseline in all samples and higher expression of the gene is observed in a subset of tumors experiencing epigenetic dysregulation. We reviewed the distribution of expression of each gene across TCGA samples and discarded those where we observed only a unimodal distribution with no significant right-hand tail, as this distribution may (as a non-limiting example) more likely characterize genes that have a low baseline of expression in normal tissues.

This left us with a remaining gene list of >630 genes that was highly enriched for genes involved in testis-specific biological processes and development. Because many of these genes produce different isoforms, these genes mapped to >1,200 proteins using the UNIPROT mapping service. In addition to the genes that met our strict computational criteria, we added several genes that have previously been identified in the scientific literature as cancer testes antigens.

To identify the peptides that are likely to be presented as cell surface antigens by MHC Class I proteins, we used a sliding window to parse each of these proteins into its constituent 8-11 amino acid sequences. We processed these peptides and their flanking sequences with the HLA peptide presentation deep learning model to calculate the likelihood of presentation of each peptide at expression levels between five TPM, which approximately corresponds to one transcript per cell [3], to 200 TPM (i.e., a high level of expression). We considered a peptide a putative HLA-PEPTIDE target if its probability of presentation calculated by our model was greater than 0.1 in 10 or more patients in the TCGA dataset with expression 5 TPM or greater.

The results are shown in Table A. From this example, there are >1,800 HLA-PEPTIDE targets across ˜400 genes and 25 analyzed HLA alleles. For clarity, each HLA-PEPTIDE was assigned a target number in Table A. For example, HLA-PEPTIDE target 1 is HLA-A*01:01_EVDPIGHLY, HLA-PEPTIDE target 2 is HLA-A*29:02_FVQENYLEY, and so forth.

Collectively, this list of HLA-PEPTIDE targets is expected to be a significant contribution to the state of knowledge of cancer specific targets. In summary, the example provides a large set of tumor-specific HLA-PEPTIDEs that can be pursued as candidate targets for ABP research and development.

### REFERENCES

1. Consortium, G T., The Genotype-Tissue Expression (GTEx) project. Nat Genet, 2013. 45(6): p. 580-5.
2. Huang da, W., B. T. Sherman, and R. A. Lempicki, Systematic and integrative analysis of large gene lists using DAVID bioinformatics resources. Nat Protoc, 2009. 4(1): p. 44-57.
3. Shapiro, E., T. Biezuner, and S. Linnarsson, Single-cell sequencing-based technologies will revolutionize whole-organism science. Nat Rev Genet, 2013. 14(9): p. 618-30.

### Example 2: Initial Validation of Predicted HLA-PEPTIDE Complexes

As an initial assessment to validate the predicted HLA-PEPTIDE targets arising from the above described approach, we evaluated public databases and selected literature for reports of these targets as having been previously identified by various assay techniques, including HLA binding affinity measurements, HLA peptide mass-spectrometry, as well as measures of T cell responses. Two comprehensive databases containing assay result annotations for HLA-PEPTIDE pairs were used: IEDB (Vita et al., 2015) and Tantigen (Olsen et al., 2017). We determined that 19 (15 unique across genes) of the computationally predicted targets were previously reported in the databases, many in genes (e.g., cancer testis antigens) that have long been the subject of study in cancer immunology. See Table B.

TABLE B

| | | Found in | | |
|---|---|---|---|---|
| Protein | | IEDB or | IEDB | Tantigen |
| Name | HLA-PEPTIDE | Tantigen | Status | Status |
| MAGA3 | HLA-A*01:01_EVDPIGHLY TRUE | Found | Found | |
| MAGA3 | HLA-A*29:02_FVQENYLEY | TRUE | Found | Not found |
| MAGA3 | HLA-A*29:02_LVHFLLLKY | TRUE | Found | Not found |
| MAGA3 | HLA-B*44:03_MEVDPIGHLY TRUE | Not found | Found | |
| MAGA6 | HLA-A*29:02_FVQENYLEY | TRUE | Found | Not found |
| MAGA6 | HLA-A*29:02_LVHFLLLKY | TRUE | Found | Not found |
| MAGA4 | HLA-A*01:01_EVDPASNTY | TRUE | Not found | Found |
| MAGA1 | HLA-A*02:01_KVLEYVIKV | TRUE | Found | Found |
| MAGAC | HLA-A*29:02_LVHFLLLKY | TRUE | Found | Not found |
| MAGAC | HLA-A*29:02_LVQENYLEY | TRUE | Found | Not found |
| SSX1 | HLA-C*04:01_AFDDIATYF | TRUE | Found | Not found |
| MAGA4 | HLA-A*29:02_WVQENYLEY | TRUE | Found | Not found |
| MAGB2 | HLA-A*02:01_GVYDGEEHSV | TRUE | Found | Not found |
| MAGA1 | HLA-A*03:01_SLFRAVITK | TRUE | Found | Found |
| MAGA4 | HLA-A*11:01_ALAETSYVK | TRUE | Found | Not found |
| SAGE1 | HLA-A*24:02_LYATVIHDI | TRUE | Not found | Found |
| PASD1 | HLA-A*02:01_QLLDGFMITL | TRUE | Found | Not found |
| MAGA8 | HLA-A*29:02_WVQENYLEY | TRUE | Found | Not found |
| MAGAC | HLA-A*29:02_STLPTTINY | TRUE | Found | Not found |

Additional limited literature review was carried out for peptides not found in the above public databases. The following peptides were identified, as shown in Table C:

TABLE C

| | | HLA/peptide known | HLA/peptide known status in |
|---|---|---|---|
| | Protein | status IEDB or | literature (preliminary) if |
| HLA allele/peptide complex | Name | Tantig 2017 | not in IEDB or Tantigen |
| HLA-A*01:01_NTDNNLAVY | KKLC1 | Not known | WO 2017/089756 A1 |
| | | | (Stevanovićetal., 2017) |
| HLA-B*35:01_YPAPLESLDY | PRA10 | Not known | W02008118017 A2 |
| HLA-A*11:01_ATLENLLSH | PRAM4 | Not known | W02008118017 A2 |
| HLA-B*51:01_DALLAQKV | PRA12 | Not known | W02008118017 A2 |
| HLA-B*44:03_SESDLKHLSW | PRA12 | Not known | W02008118017 A2 |
| HLA-A*11:01_ATLENLLSH | PRAM9 | Not known | W02008118017 A2 |
| HLA-A*02:07_TLDEYLTYL | PRAM9 | Not known | W02008118017 A2 |

One notable example from Table C was KKLC1 HLA-A*01:01_NTDNNLAVY. Kita-kyushu lung cancer antigen-1 (KK-LC-1; CT83) is a cancer testis antigen (CTA) that has been shown to be widely expressed in many different cancer types. It was originally discovered based on a cloned CTL to KK-LC-1 peptide 76-84—RQKRILVNL (Fukuyama et al., 2006). More recently Stevanovic et al., 2017 revealed another peptide from KK-LC-1 recognized by a CTL in a patient with cervical cancer, the predicted peptide KK-LC-1 52-60 NTDNNLAVY The corresponding TCR for this CTL is now listed on the NIH website https://www.ott.nih.gov/technology/e-153-2016/ and the peptide is listed in WO 2017/089756 A1, herein incorporated by reference, in its entirety, for all purposes.

This example highlights the expected value of predicted HLA-PEPTIDE targets in Table A: Although no information on which CTA HLA-PEPTIDE targets were previously known was incorporated in the prediction, the analysis yielded many targets that were described in the literature, indicating that many of the novel targets can likewise be validated experimentally and ultimately serve as targets for one or more ABPs.

### REFERENCES

Fukuyama, T., Hanagiri, T., Takenoyama, M., Ichiki, Y, Mizukami, M., So, T., Sugaya, M., So, T., Sugio, K., and Yasumoto, K. (2006). Identification of a new cancer/germline gene, KK-LC-1, encoding an antigen recognized by autologous CTL induced on human lung adenocarcinoma. Cancer Res. 66, 4922-4928.
Olsen, L. R., Tongchusak, S., Lin, H., Reinherz, E. L., Brusic, V., and Zhang, G. L. (2017). TANTIGEN: a comprehensive database of tumor T cell antigens. Cancer Immunol. Immunother. CII 66, 731-735.
Stevanovic, S., Pasetto, A., Helman, S. R., Gartner, J. J., Prickett, T. D., Howie, B., Robins, H. S., Robbins, P F., Klebanoff, C. A., Rosenberg, S. A., et al. (2017). Landscape of immunogenic tumor antigens in successful immunotherapy of virally induced epithelial cancer. Science 356, 200-205.
Vita, R., Overton, J. A., Greenbaum, J. A., Ponomarenko, J., Clark, J. D., Cantrell, J. R., Wheeler, D. K., Gabbard, J. L., Hix, D., Sette, A., et al. (2015). The immune epitope database (IEDB) 3.0. Nucleic Acids Res. 43, D405-412.

### Example 3: Identification of Predicted HLA-PEPTIDE Complexes

Next, HLA-peptide targets from proteins of seven genes were identified: AFP, KKLC-1, MAGE-A4, MAGE-A10, MART-1, NY-ESO-1, and WT1.

To identify peptides that are likely to be presented as cell surface antigens by MHC Class I proteins, a sliding window was used to parse each of these proteins into its constituent 8-11 amino acid sequences. These peptides and their flanking sequences were then processed with the HLA peptide presentation deep learning model (see PCT/US2016/067159 and Example 1 above) to calculate the likelihood of presentation of each peptide at an expression level of 100 TPM (high expression) for each of 64 Class I HLA types. Potential modeling artifacts were removed that could give stronger scores to certain HLAs due to training data biases by quantile normalizing model scores for each HLA so that each HLA present scores from the same distribution. In the normalization, the seven target genes as well as 50 randomly selected genes were included to control for HLA allele sequence preferences. A gene was considered likely to be presented if the model normalized score was higher than 0.00075, which was chosen based on the presentation scores of peptides known to be presented in the literature.

The results are shown in Table A (cont.). Target numbers were assigned to each HLA-PEPTIDE target as described in Example 1.

### Example 4: Validation of Predicted HLA-PEPTIDE Complexes

The presence of peptides from the HLA-PEPTIDE complexes of Table A were determined using mass spectrometry (MS) on tumor samples known to be positive for each given HLA allele from the respective HLA-PEPTIDE complex.

Isolation of HLA-peptide molecules was performed using classic immunoprecipitation (IP) methods after lysis and solubilization of the tissue sample (1-4). Fresh frozen tissue was first frozen in liquid nitrogen and pulverized (CryoPrep; Covaris, Woburn, Mass.). Lysis buffer (1% CHAPS, 20 mM Tris-HCl, 150 mM NaCl, protease and phosphatase inhibitors, pH=8) was added to solubilize the tissue and 1/10^{th} of the sample was aliquoted for proteomics and genomic sequencing efforts. The remainder of the sample was spun at 4C for 2 hrs to pellet debris. The clarified lysate was used for the HLA specific IP.

Immunoprecipitation was performed using antibodies coupled to beads where the antibody was specific for HLA molecules. For a pan-Class I HLA immunoprecipitation, the antibody W6/32 (5) was used, for Class II HLA—DR, antibody L243 (6) was used. Antibody was covalently attached to NHS-sepharose beads during overnight incubation. After covalent attachment, the beads were washed and aliquoted for IP. Additional methods for IP can be used including but not limited to Protein A/G capture of antibody, magnetic bead isolation, or other methods commonly used for immunoprecipitation.

The lysate was added to the antibody beads and rotated at 4C overnight for the immunoprecipitation. After immunoprecipitation, the beads were removed from the lysate and the lysate was stored for additional experiments, including additional IPs. The IP beads are washed to remove non-specific binding and the HLA/peptide complex was eluted from the beads with 2N acetic acid. The protein components were removed from the peptides using a molecular weight spin column. The resultant peptides were taken to dryness by SpeedVac evaporation and can be stored at −20 C prior to MS analysis.

Dried peptides were reconstituted in HPLC buffer A and loaded onto a C-18 microcapillary HPLC column for gradient elution in to the mass spectrometer. A gradient of 0-40% B (solvent A—0.1% formic acid, solvent B— 0.1% formic acid in 80% acetonitrile) in 180 minutes was used to elute the peptides into the Fusion Lumos mass spectrometer (Thermo). MS1 spectra of peptide mass/charge (m/z) were collected in the Orbitrap detector with 120,000 resolution followed by 20 MS2 scans. Selection of MS2 ions was performed using data dependent acquisition mode and dynamic exclusion of 30 sec after MS2 selection of an ion. Automatic gain control (AGC) for MS1 scans was set to 4×105 and for MS2 scans was set to 1×104. For sequencing HLA peptides, +1, +2 and +3 charge states can be selected for MS2 fragmentation. This was commonly referred to as Targeted Mass Spectrometry and was performed in either a qualitative manner or can be quantitative. Quantitation methods require each peptide to be quantitated to be synthesized using heavy labeled amino acids. (Doerr 2013)

MS2 spectra from each analysis were searched against a protein database using Comet (7-8) and the peptide identification was scored using Percolator (9-11) or using the integrated de novo sequencing and database search algorithm of PEAKS.

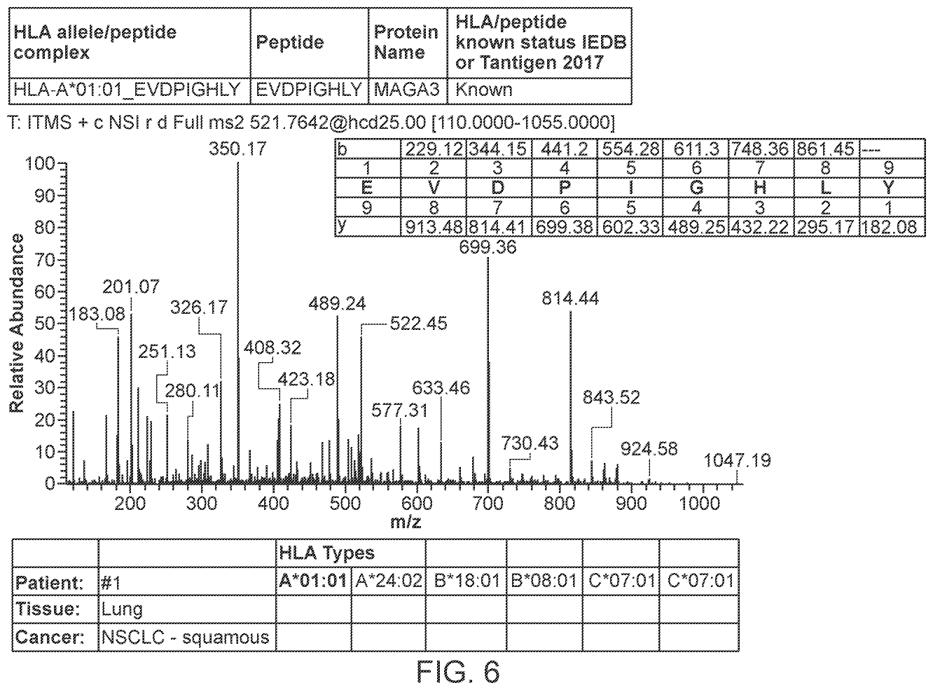

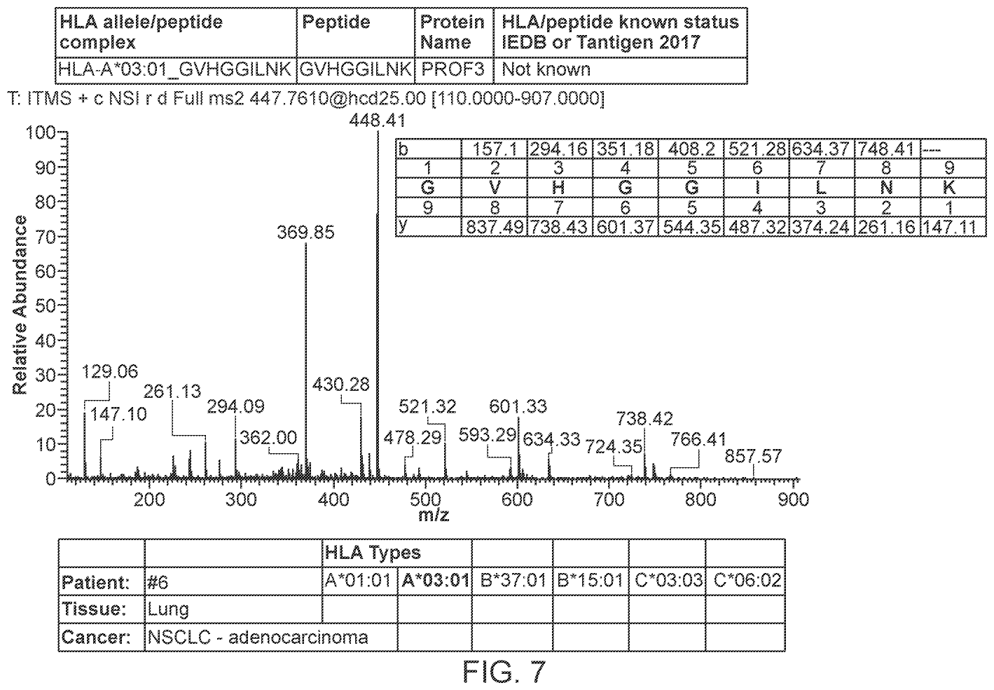

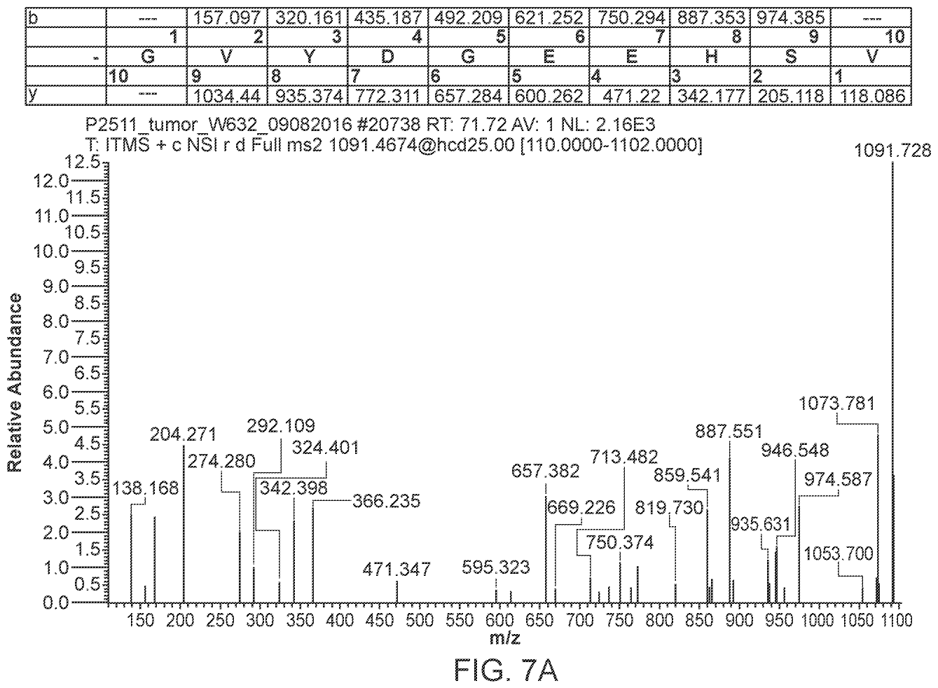

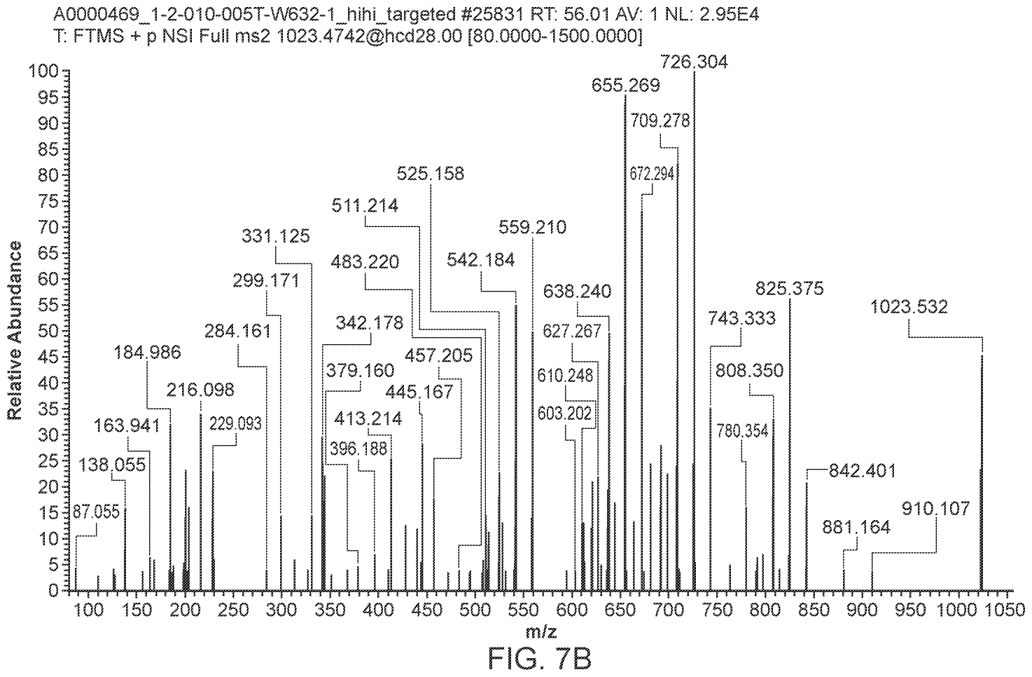

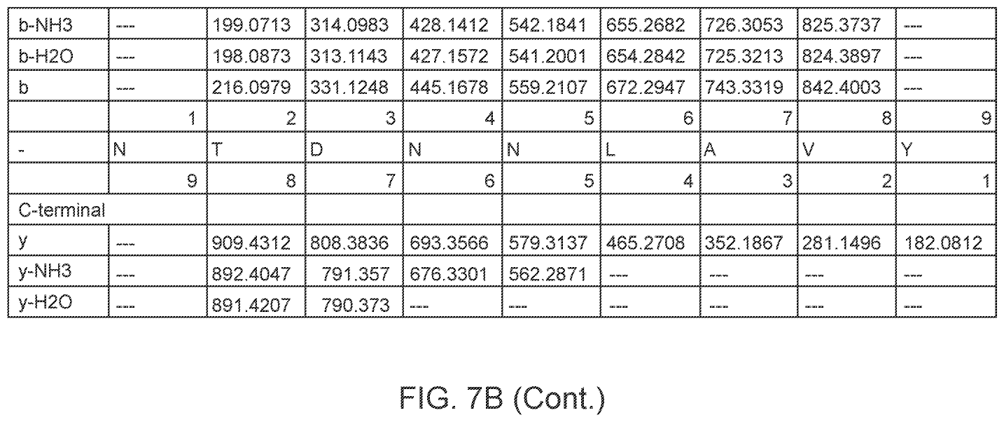

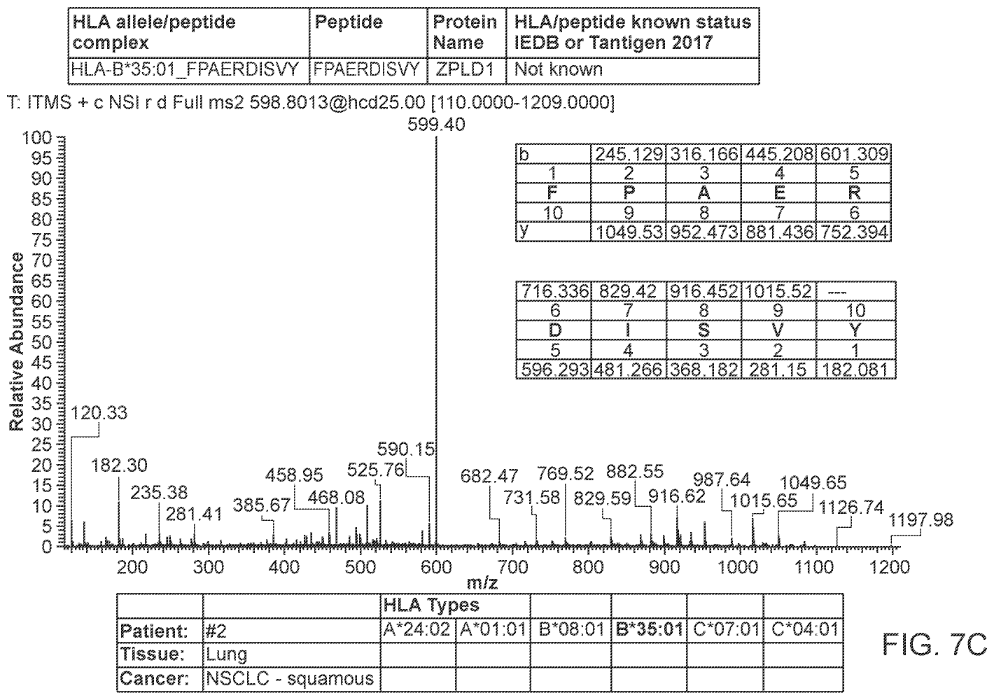

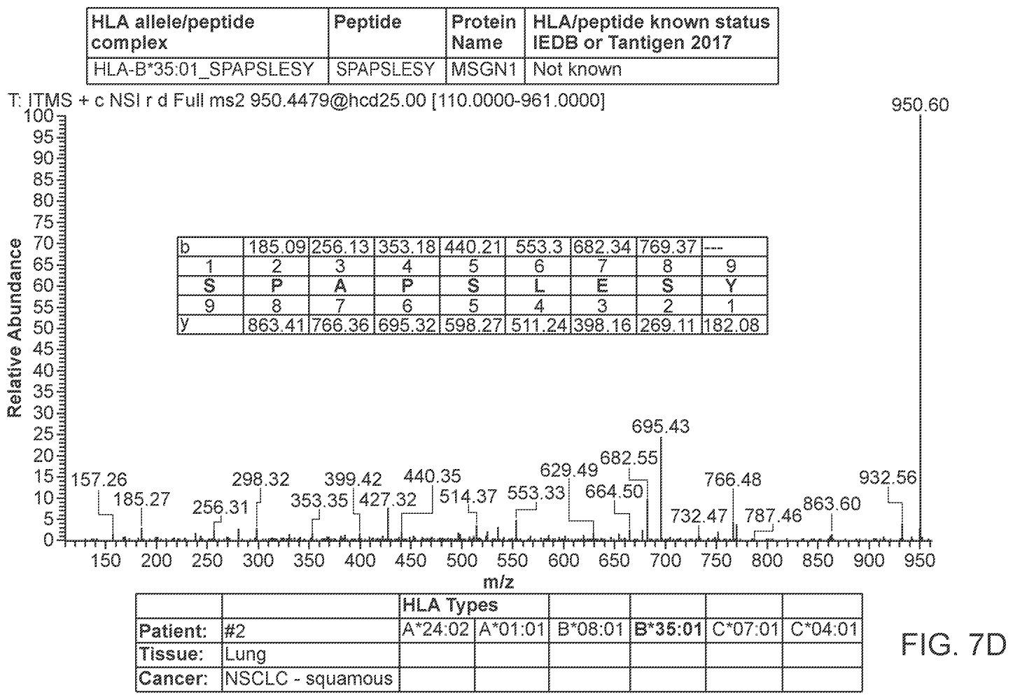

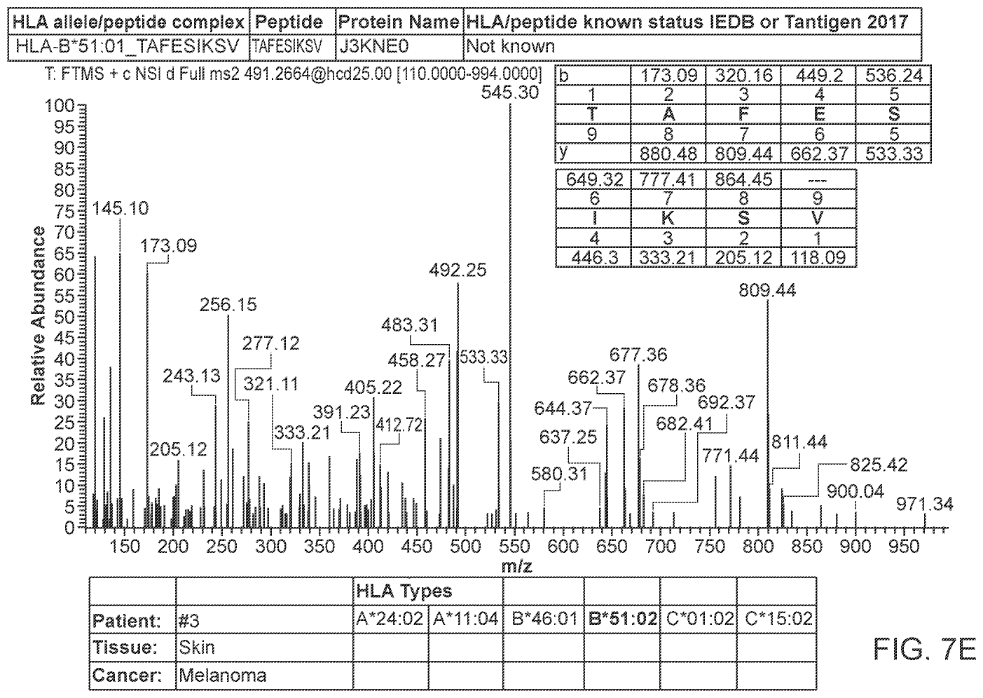

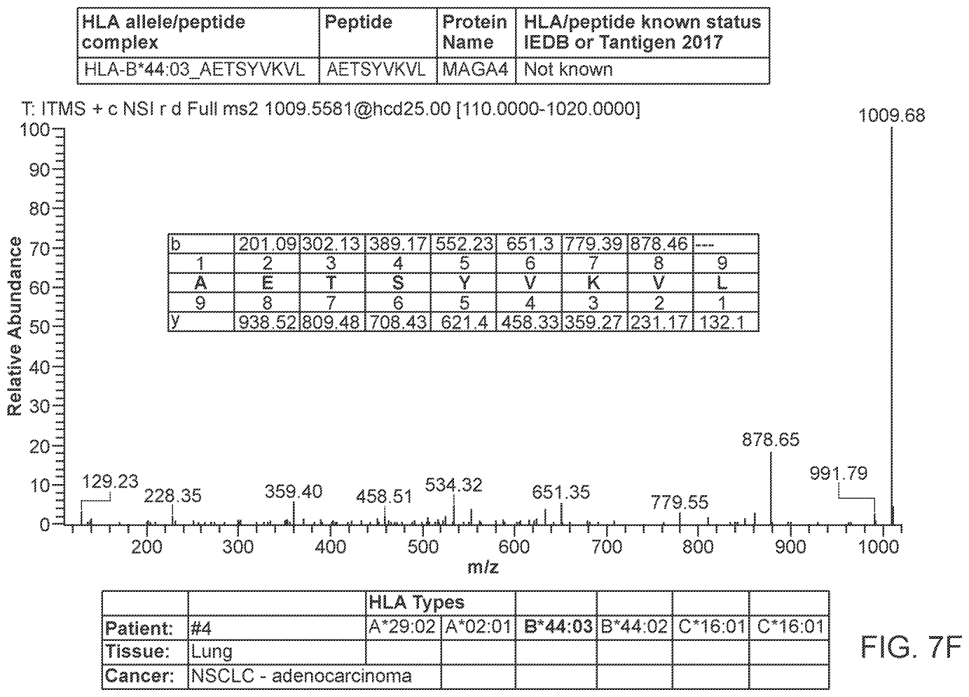

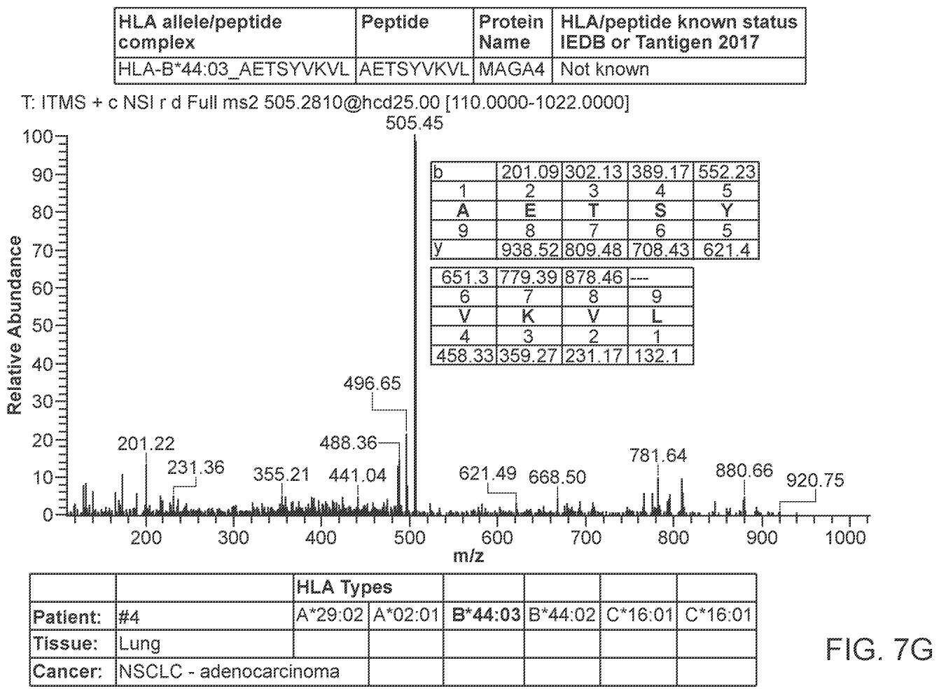

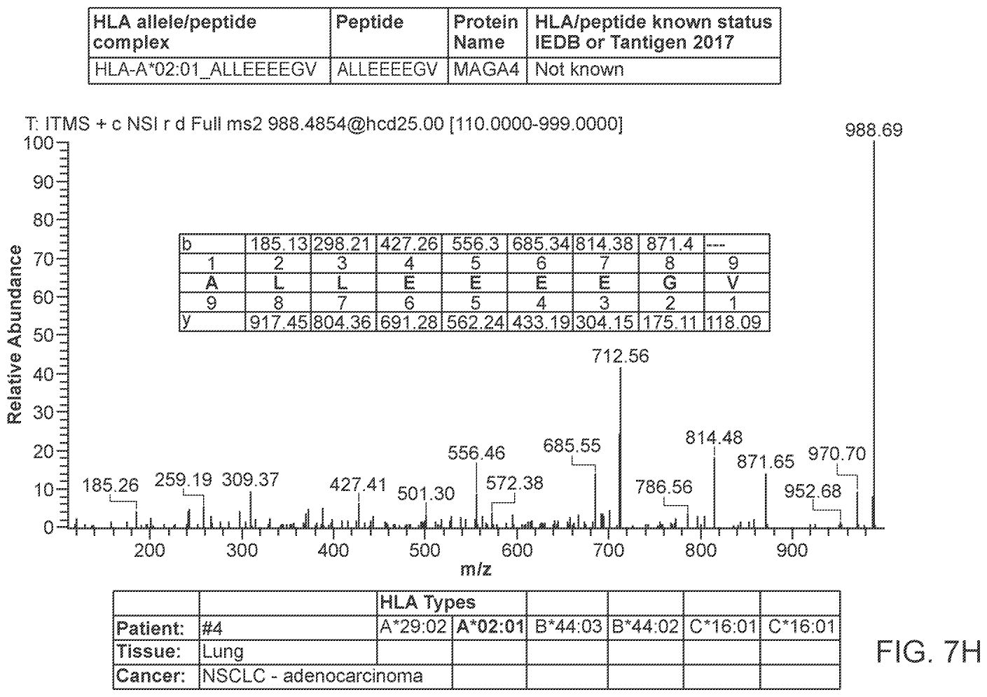

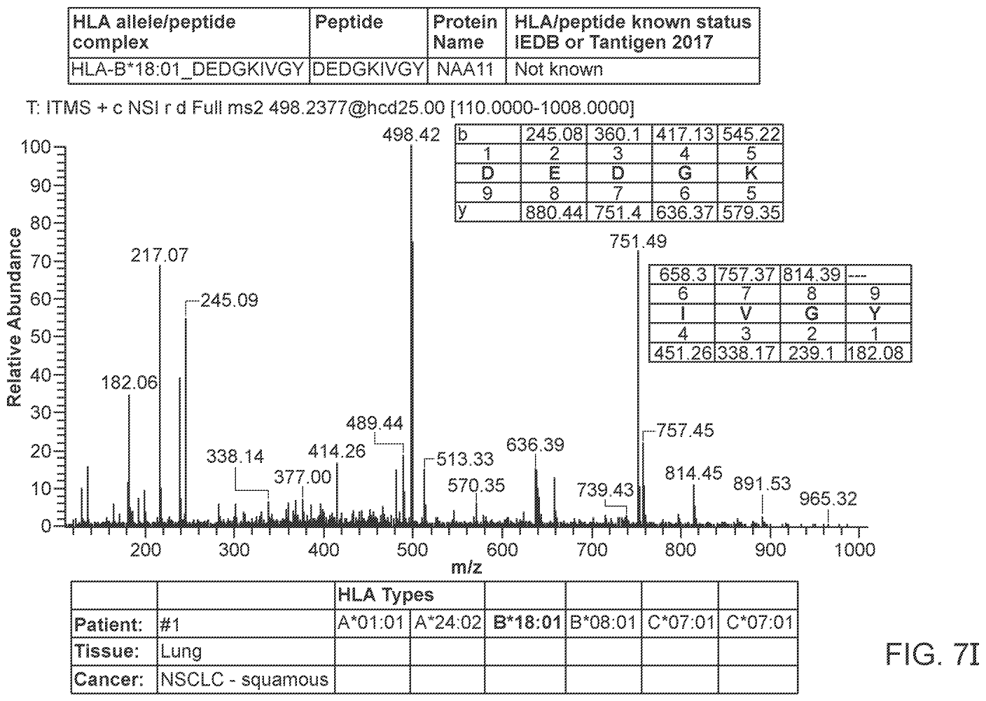

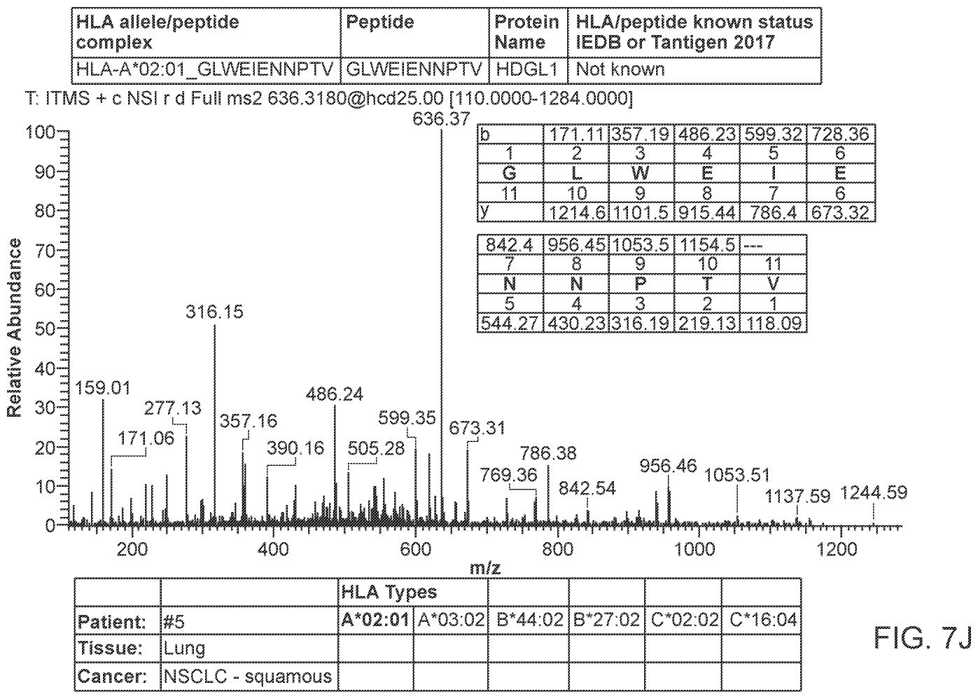

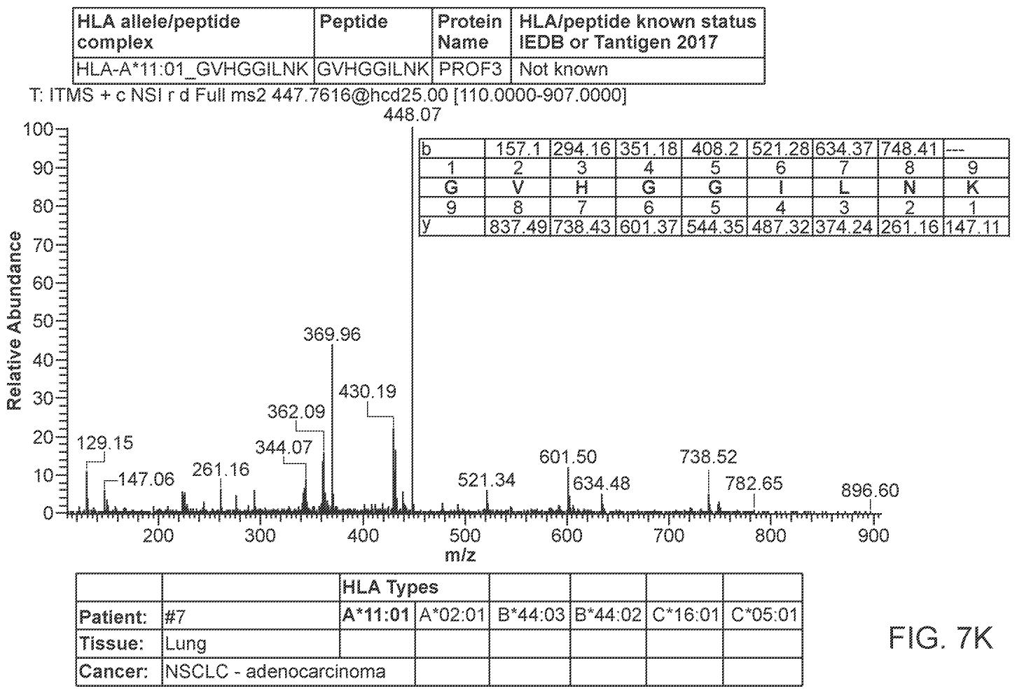

The presence of peptides from HLA-PEPTIDE complexes was determined using mass spectrometry (MS) on various tumor samples known to be positive for each given HLA allele from the respective HLA-PEPTIDE complex. Representative spectra data forfor selected HLA-restricted peptides is shown in FIGS. 6 and 7. Each spectra contains the peptide fragmentation information as well as information related to the patient sample, including HLA types.

The spontaneous modification of amino acids can occur to many amino acids. Cysteine is especially susceptible to this modification and can be oxidized or modified with a free cysteine. Additionally N-terminal glutamine amino acids can be converted to pyro-glutamic acid. Since each of these modifications results in a change in mass, they can be definitively assigned in the MS2 spectra. To use these peptides in preparation of ABPs the peptide may need to contain the same modification as seen in the mass spectrometer. These modifications can be created using simple laboratory and peptide synthesis methods (Lee et al.; Ref 14).

### REFERENCES

(1) Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992. 255: 1261-1263.
(2) Zarling A L, Polefrone J M, Evans A M, Mikesh L M, Shabanowitz J, Lewis S T, Engelhard V H, Hunt D F. Identification of class I MHC-associated phosphopeptides as targets for cancer immunotherapy. Proc Natl Acad Sci USA. 2006 Oct. 3; 103(40):14889-94.
(3) Bassani-Sternberg M, Pletscher-Frankild S, Jensen L J, Mann M. Mass spectrometry of human leukocyte antigen class I peptidomes reveals strong effects of protein abundance and turnover on antigen presentation. Mol Cell Proteomics. 2015 March; 14(3):658-73. doi: 10.1074/mcp.M114.042812.
(4) Abelin J G, Trantham P D, Penny S A, Patterson A M, Ward S T, Hildebrand W H, Cobbold M, Bai D L, Shabanowitz J, Hunt D F. Complementary IMAC enrichment methods for HLA-associated phosphopeptide identification by mass spectrometry. Nat Protoc. 2015 September; 10(9):1308-18. doi: 10.1038/nprot.2015.086. Epub 2015 Aug. 6
(5) Barnstable C J, Bodmer W F, Brown G, Galfre G, Milstein C, Williams A F, Ziegler A. Production of monoclonal antibodies to group A erythrocytes, HLA and other human cell surface antigens-new tools for genetic analysis. Cell. 1978 May; 14(1):9-20.
(6) Goldman J M, Hibbin J, Kearney L, Orchard K, Th'ng K H. HLA-DR monoclonal antibodies inhibit the proliferation of normal and chronic granulocytic leukaemia myeloid progenitor cells. Br J Haematol. 1982 November; 52(3):411-20.
(7) Eng J K, Jahan T A, Hoopmann M R. Comet: an open-source MS/MS sequence database search tool. Proteomics. 2013 January; 13(1):22-4. doi: 10.1002/pmic.201200439. Epub 2012 Dec. 4.
(8) Eng J K, Hoopmann M R, Jahan T A, Egertson J D, Noble W S, MacCoss M J. A deeper look into Comet-implementation and features. J Am Soc Mass Spectrom. 2015 November; 26(11):1865-74. doi: 10.1007/s13361-015-1179-x. Epub 2015 Jun. 27.
(9) Lukas Käll, Jesse Canterbury, Jason Weston, William Stafford Noble and Michael J. MacCoss. Semi-supervised learning for peptide identification from shotgun proteomics datasets. Nature Methods 4:923-925, November 2007
(10) Lukas Käll, John D. Storey, Michael J. MacCoss and William Stafford Noble. Assigning confidence measures to peptides identified by tandem mass spectrometry. Journal of Proteome Research, 7(1):29-34, January 2008
(11) Lukas Käll, John D. Storey and William Stafford Noble. Nonparametric estimation of posterior error probabilities associated with peptides identified by tandem mass spectrometry. Bioinformatics, 24(16):i42-i48, August 2008
(12) Doerr, A. (2013) Mass Spectrometry-based targeted proteomics. Nature Methods, 10, 23.
(13) Lindsay K. Pino, Brian C. Searle, James G Bollinger, Brook Nunn, Brendan MacLean & M. J. MacCoss (2017) The Skyline ecosystem: Informatics for quantitative mass spectrometry proteomics. Mass Spectrometry Reviews.
(14) Lee W Thompson; Kevin T Hogan; Jennifer A Caldwell; Richard A Pierce; Ronald C Hendrickson; Donna H Deacon; Robert E Settlage; Laurence H Brinckerhoff; Victor H Engelhard; Jeffrey Shabanowitz; Donald F Hunt; Craig L Slingluff. Preventing the spontaneous modification of an HLA-A2-restricted peptide at an N-terminal glutamine or an internal cysteine residue enhances peptide antigenicity. Journal of Immunotherapy (Hagerstown, Md.: 1997). 27(3):177-83, May 2004.
2017) or other method to analyze predicted fragment ions.

The presence of multiple peptides from the predicted HLA-PEPTIDE complexes is determined using mass spectrometry (MS) on various tumor samples known to be positive for each given HLA allele from the respective HLA-PEPTIDE complex.

The spontaneous modification of amino acids can occur to many amino acids. Cysteine is especially susceptible to this modification and can be oxidized or modified with a free cysteine. Additionally N-terminal glutamine amino acids can be converted to pyro-glutamic acid. Since each of these modifications results in a change in mass, they can be definitively assigned in the MS2 spectra. To use these peptides in preparation of ABPs the peptide may need to contain the same modification as seen in the mass spectrometer. These modifications can be created using simple laboratory and peptide synthesis methods (Lee et al.; Ref 14).

### REFERENCES

(1) Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992. 255: 1261-1263.
(2) Zarling A L, Polefrone J M, Evans A M, Mikesh L M, Shabanowitz J, Lewis S T, Engelhard V H, Hunt D F. Identification of class I MHC-associated phosphopeptides as targets for cancer immunotherapy. Proc Natl Acad Sci USA. 2006 Oct. 3; 103(40):14889-94.
(3) Bassani-Sternberg M, Pletscher-Frankild S, Jensen L J, Mann M. Mass spectrometry of human leukocyte antigen class I peptidomes reveals strong effects of protein abundance and turnover on antigen presentation. Mol Cell Proteomics. 2015 March; 14(3):658-73. doi: 10.1074/mcp.M114.042812.
(4) Complementary IMAC enrichment methods for HLA-associated phosphopeptide identification by mass spectrometry. 2015 September; 10(9):1308-18. doi: 10.1038/nprot.2015.086. Epub 2015 Aug. 6
(5) Barnstable C J, Bodmer W F, Brown G, Galfre G, Milstein C, Williams A F, Ziegler A. Production of monoclonal antibodies to group A erythrocytes, HLA and other human cell surface antigens-new tools for genetic analysis. Cell. 1978 May; 14(1):9-20.
(6) Goldman J M, Hibbin J, Kearney L, Orchard K, Th'ng K H. HLA-D R monoclonal antibodies inhibit the proliferation of normal and chronic granulocytic leukaemia myeloid progenitor cells. Br J Haematol. 1982 November; 52(3):411-20.
(7) Eng J K, Jahan T A, Hoopmann M R. Comet: an open-source MS/MS sequence database search tool. Proteomics. 2013 January; 13(1):22-4. doi: 10.1002/pmic.201200439. Epub 2012 Dec. 4.
(8) Eng J K, Hoopmann M R, Jahan T A, Egertson J D, Noble W S, MacCoss M J. A deeper look into Comet-implementation and features. J Am Soc Mass Spectrom. 2015 November; 26(11):1865-74. doi: 10.1007/s13361-015-1179-x. Epub 2015 Jun. 27.
(9) Lukas Käll, Jesse Canterbury, Jason Weston, William Stafford Noble and Michael J. MacCoss. Nature Methods 4:923-925, November 2007
(10) Lukas Käll, John D. Storey, Michael J. MacCoss and William Stafford Noble. Journal of Proteome Research, 7(1):29-34, January 2008
(11) Lukas Käll, John D. Storey and William Stafford Noble. Nonparametric estimation of posterior error probabilities associated with peptides identified by tandem mass spectrometry. Bioinformatics, 24(16):i42-i48, August 2008
(12) Doerr, A. (2013) Mass Spectrometry-based targeted proteomics. Nature Methods, 10, 23.
(13) Lindsay K. Pino, Brian C. Searle, James G Bollinger, Brook Nunn, Brendan MacLean & M. J. MacCoss (2017) The Skyline ecosystem: Informatics for quantitative mass spectrometry proteomics. Mass Spectrometry Reviews.
(14) Lee W Thompson; Kevin T Hogan; Jennifer A Caldwell; Richard A Pierce; Ronald C Hendrickson; Donna H Deacon; Robert E Settlage; Laurence H Brinckerhoff; Victor H Engelhard; Jeffrey Shabanowitz; Donald F Hunt; Craig L Slingluff. Preventing the spontaneous modification of an HLA-A2-restricted peptide at an N-terminal glutamine or an internal cysteine residue enhances peptide antigenicity. Journal of Immunotherapy (Hagerstown, Md.: 1997). 27(3):177-83, May 2004.

### Example 6: Identification of Antibodies or Antigen-Binding Fragments Thereof that Bind HLA-PEPTIDE Complexes

Overview

The following exemplification demonstrates that antibodies (Abs) can be identified that recognize tumor-specific HLA-restricted peptides. The overall epitope that is recognized by such Abs generally comprises a composite surface of both the peptide as well as the HLA protein presenting that particular peptide. Abs that recognize HLA complexes in a peptide-specific manner are often referred to as T cell receptor (TCR)-like Abs or TCR-mimetic Abs. The HLA-PEPTIDE target antigens that were selected for antibody discovery are HLA-A*01:01_NTDNNLAVY (Target X in Table A, designated as “G2”) and HLA-A*02:01_LLASSILCA (Target X in Table A, designated as “G7”). Cell surface presentation of these HLA-PEPTIDE antigens was confirmed by mass spectrometry analysis of HLA complexes obtained from tumor samples, as described in Example 4.

Generation of HLA-PEPTIDE Target Complexes and Counterscreen Peptide-HLA Complexes, and Stability Analysis

The HLA-PEPTIDE targets G2 and G7, as well as counterscreen negative control peptide-HLAs, were produced recombinantly using conditional ligands for HLA molecules using established methods. In all, 18 counterscreen HLA-peptides were generated for each of the G2 and G7 targets.

Overall Design of Phage Library Screening

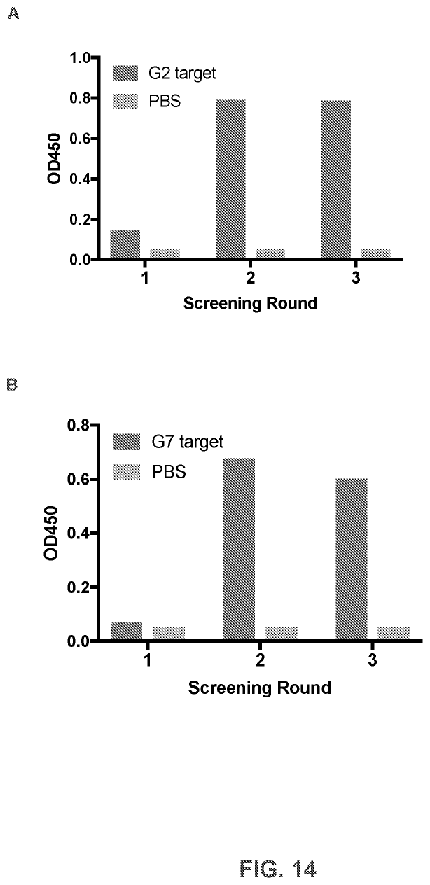

The highly diverse SuperHuman 2.0 synthetic naïve scFv library from Distributed Bio Inc (7.6e10 total diversity on ultra-stable and diverse VH/VL scaffolds) was used for phage display. The phage library was initially depleted with 18 pooled negative pHLA complexes (the “complete pool”) followed by three to four rounds of bead-based phage panning with the target pHLA complex using established protocols to identify scFv binders to HLA-PEPTIDE targets G2 and G7, respectively. The phage titer was determined at every round of panning to establish removal of non-binding phage. Phage ELISA results are shown in FIGS. 14A and 14B. There was an enrichment of bound phage in later rounds of panning for each of the G2 and G7 targets The output phage supernatant was also tested for target binding by ELISA.

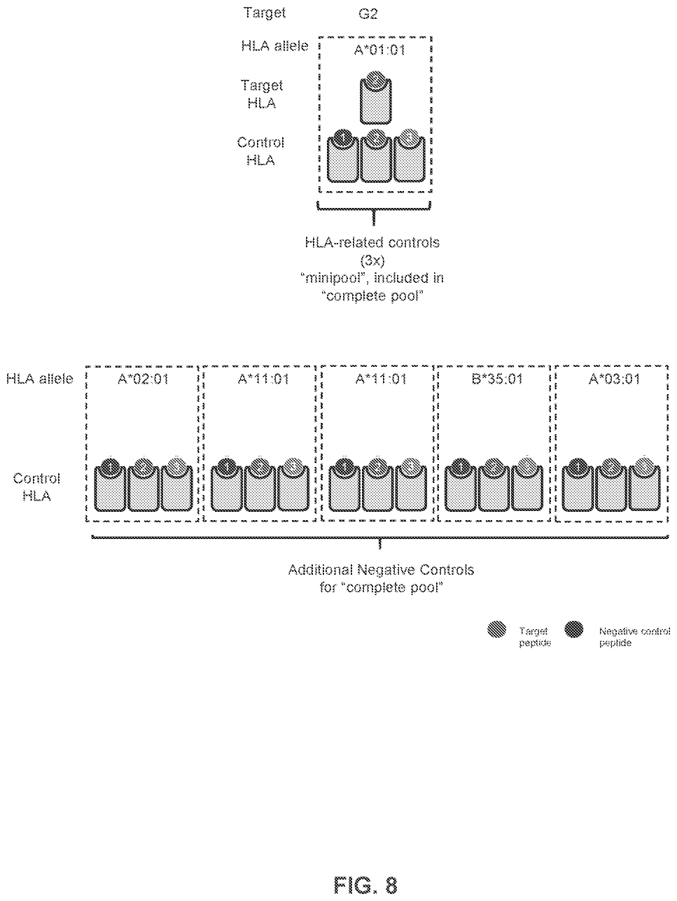

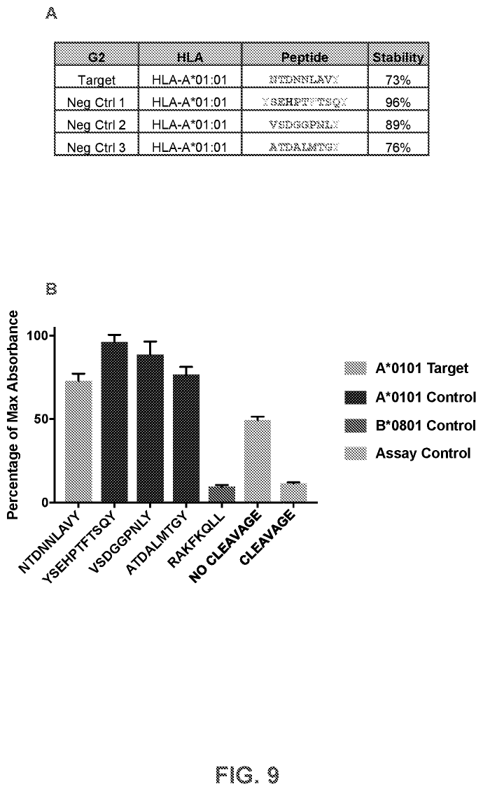

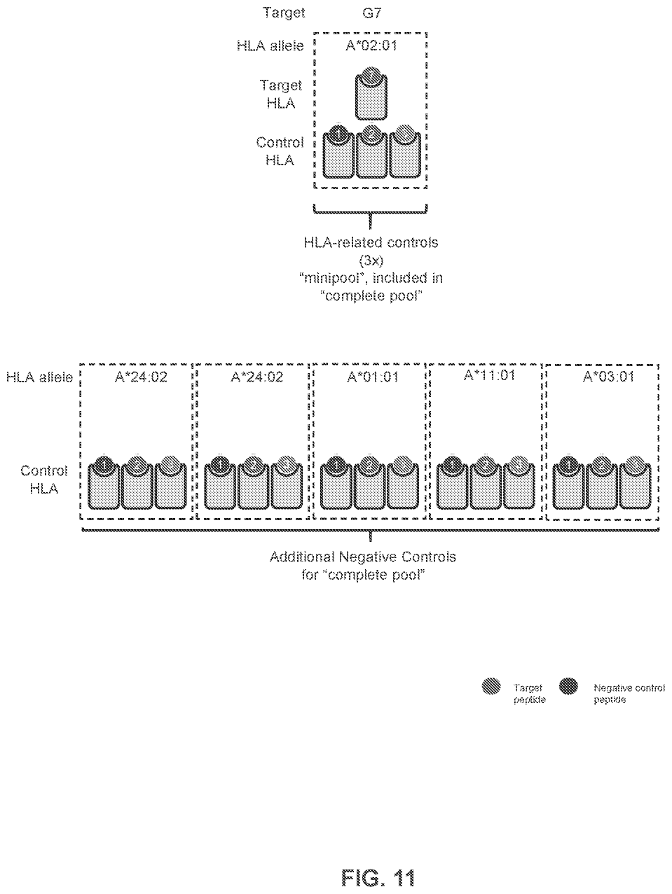

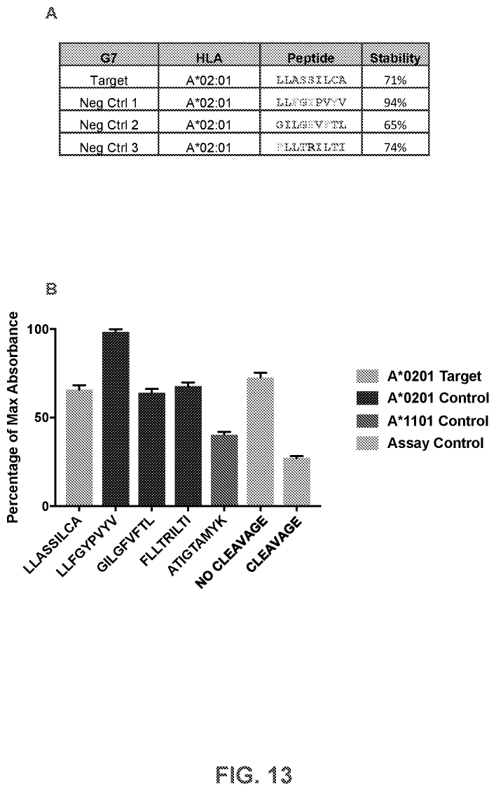

The design of target screen 1 for the G2 target is shown in FIG. 8. Similarly, the design of target screen 2 for the G7 target is shown in FIG. 11. Briefly, for each target, three “minipool” counterscreen peptides were selected for their ability to bind the same HLA allele as the target and also to have significantly different ABP-facing features such as charge, bulk, aromatic, or hydrophobic residues. See FIG. 9A for G2 and FIG. 13A for G7. In addition, additional counterscreen peptide-HLA complexes, featuring distinct restricted peptide sequences and different HLA alleles were generated. The 15 additional counterscreen HLA-peptides plus the three “minipool” HLA-peptides formed a “complete pool” of 18 total counterscreen HLA-peptide complexes.

Generation of Peptide-HLA Complexes

α-, and β2 microglobulin chain of various human leukocyte antigens (HLA) were expressed separately in BL21 competent E. coli cells (New England Biolabs) using established procedures (Garboczi, Hung, & Wiley, 1992). Following auto-induction, cells were lysed via sonication in Bugbuster® plus benzonase protein extraction reagent (Novagen). The resulting inclusion bodies were washed and sonicated in wash buffer with and without 0.5% Triton X-100 (50 mM Tris, 100 mM NaCl, 1 mM EDTA). After the final centrifugation, inclusion pellets were dissolved in urea solution (8 M urea, 25 mM MES, 10 mM EDTA, 0.1 mM DTT, pH 6.0). Bradford assay (Biorad) was used to quantify the concentration and the inclusion bodies were stored at −80° C.

HLA complexes were obtained by refolding of recombinantly produced subunits and a synthetically obtained peptide using established procedures. (Garboczi et al., 1992). Briefly, the purified α and β2 microglobulin chains were refolded in refold buffer (100 mM Tris pH 8.0, 400 mM L-Arginine HCl, 2 mM EDTA, 50 mM oxidized glutathione, 5 mM reduced glutathione, protease inhibitor tablet) with the restricted peptide of choice. In some experiments, the restricted peptide of choice was a conditional ligand peptide, which is cleavable upon exposure to a conditional stimulus. In some experiments, the restricted peptide of choice was the G2 or G7 target peptide, or counterscreen peptide. The refold solution was concentrated with a Vivaflow 50 or 50R crossflow cassette (Sartorius Stedim). Three rounds of dialyses in 20 mM Tris pH 8.0 were performed for at least 8 hours each. For the antibody screening and functional assays, the refolded HLA was enzymatically biotinylated using BirA biotin ligase (Avidity). Refolded protein complexes were purified using a HiPrep (16/60 Sephacryl S200) size exclusion column attached to an Akta FPLC system. Biotinylation was confirmed in a streptavidin gel-shift assay under non-reducing conditions by incubating the refolded protein with an excess of streptavidin at room temperature for 15 minutes prior to SDS-PAGE. The resulting peptide-HLA complexes were aliquoted and stored at −80° C.

Stability Analysis of the Peptide-HLA Complexes

HLA-peptide stability was assessed by conditional ligand peptide exchange and stability ELISA assay. Briefly, conditional ligand-HLA complexes were subjected to ±conditional stimulus in the presence or absence of the counterscreen or test peptides. Exposure to the conditional stimulus cleaves the conditional ligand from the HLA complex, resulting in dissociation of the HLA complex. If the counterscreen or test peptide stably binds the α1/α2 groove of the HLA complex, it “rescues” the HLA complex from disassociation.

The HLA stability ELISA was performed using established procedures. (Chew et al., 2011; Rodenko et al., 2006) A 384-well clear flat bottom polystyrene microplate (Corning) was precoated with 50 μl of streptavidin (Invitrogen) at 2 μg mL⁻¹ in PBS. Following 2 h of incubation at 37° C., the wells were washed with 0.05% Tween 20 in PBS (four times, 50 μL) wash buffer, treated with 50 μl of blocking buffer (2% BSA in PBS), and incubated 30 min at room temperature. Subsequently, 25 μl of peptide-exchanged samples that were 300× diluted with 20 mM Tris HCl/50 mM NaCl were added in quadruplicate. The samples were incubated for 15 min at RT, washed with 0.05% Tween wash buffer (4×50 μL), treated for 15 min with 25 μL of HRP-conjugated anti-β2m (1 μg mL⁻¹ in PBS) at RT, washed with 0.05% Tween wash buffer (4×50 μL), and developed for 10-15 min with 25 μL of ABTS-solution (Invitrogen), and the reactions were stopped by the addition of 12.5 μL of stop buffer (0.01% sodium azide in 0.1 M citric acid). Absorbance was subsequently measured at 415 nm using a spectrophotometer (SpectraMax i3x; Molecular Devices).

Results for the G2 counterscreen “minipool” and G2 target are shown in FIG. 9B. All three counterscreen peptides and the G2 peptide rescued the HLA complex from dissociation.

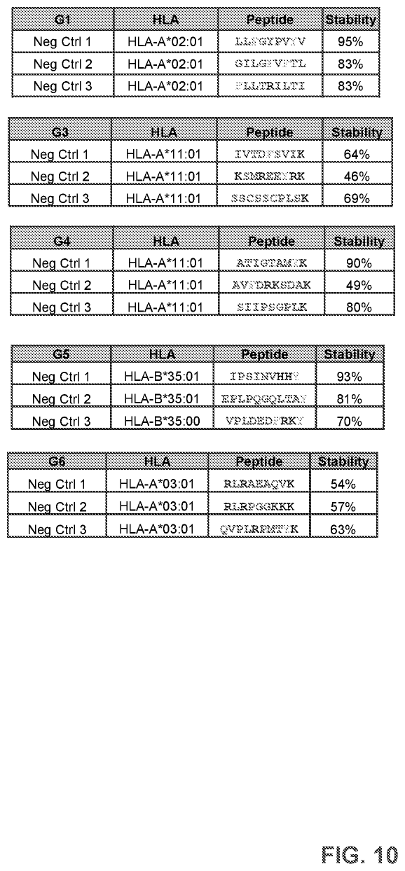

Results for the additional G2 “complete” pool counterscreen peptides are shown in FIG. 10, demonstrating that they also form stable HLA-peptide complexes.

Results for the G7 counterscreen “minipool” and G7 target are shown in FIG. 13B. All three counterscreen peptides and the G7 peptide rescued the HLA complex from dissociation.

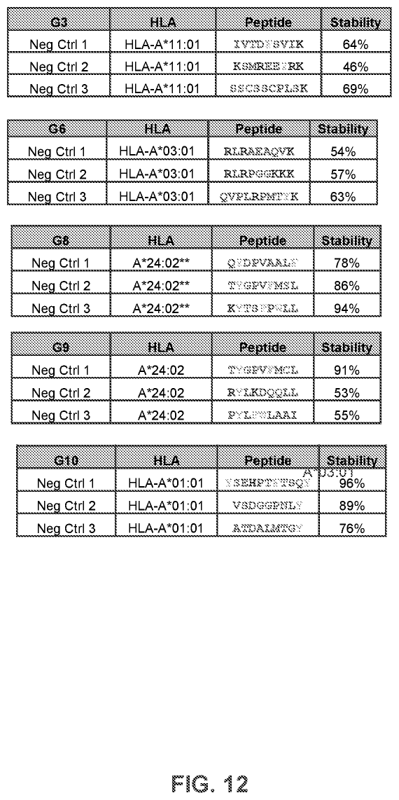

Results for the additional G7 “complete” pool counterscreen peptides are shown in FIG. 12, demonstrating that they also form stable HLA-peptide complexes.

Phage Library Screening

Phage library screening was carried out according to the overall screening design described above. Three to four rounds of bead-based panning were performed to identify scFv binders to each peptide-HLA complex. For each round of panning, an aliquot of starting phage was set aside for input titering and the remaining phage was depleted three times against Dynabead M-280 streptavidin beads (Life Technologies) followed by a depletion against Streptavidin beads pre-bound with 100 pmoles of pooled negative peptide-HLA complexes. For the first round of panning, 100 pmoles of peptide-HLA complex bound to streptavidin beads was incubated with depleted phage for 2 hours at room temperature with rotation. Three five-minute washes with 0.5% BSA in 1×PBST (PBS+0.05% Tween-20) followed by three five-minute washes with 0.5% BSA in 1×PBS were utilized to remove any unbound phage to the peptide-HLA complex bound beads. To elute the bound phage from the washed beads, 1 ml 0.1M TEA was added and incubated for 10 minutes at room temperature with rotation. The eluted phage was collected from the beads and neutralized with 0.5 ml 1M Tris-HCl pH 7.5. The neutralized phage was then used to infect log growth TG-1 cells (OD₆₀₀=0.5) and after an hour of infection at 37° C., cells were plated onto 2YT media with 100 μg/ml carbenicillin and 2% glucose (2YTCG) agar plates for output titer and bacterial growth for subsequent panning rounds. For subsequent rounds of panning, selection antigen concentrations were lowered while washes increased by amount and length of wash times at show in Table 1.

TABLE 1
Phage library screening strategy

| Round | Antigen concentration | Washes |
|---|---|---|
| R1 | 100 pmol | 3x PBST + 3x |
| | | PBS (5 min washes) |
| R2 | 25 pmol | 5 PBST (2x 30 sec, 3x 5 min) + |
| | | 5 PBS (2x 30 sec, 3x 5 |
| | | min) |
| R3 | 10 pmol | 8 PBST (4x 30 sec, 4x 5 min) + |
| | | 8 PBS (4x 30 sec, 4x 5 |
| | | min) |
| R4 | 5 pmol, 10 pmol | 30 min PBST + |
| | | 30 min PBS |

Individual scFvs were cloned from phage and sequenced by DNA Sanger sequencing (“Sequence Unique Binders”). The individual scFvs were also expressed in E. coli and periplasmic extracts (PPE) from E. coli containing the individual crude scFvs were subjected to scFv ELISA

scFv Periplasmic Extract (PPE) ELISA

The individual scFv cloned from phage obtained in the final round of panning, and expressed in E. coli, was subjected to scFv PPE ELISA as follows.

96-well and/or 384-well streptavidin coated plates (Pierce) were coated with 2 ug/ml peptide-HLA complex in HLA buffer and incubated overnight at 4° C. Plates were washed three times between each step with PBST (PBS+0.05%). The antigen coated plates were blocked with 3% BSA in PBS (blocking buffer) for 1 hour at room temperature. After washing, scFv PPEs were added to the plates and incubated at room temperature for 1 hour. Following washing, mouse anti-v5 antibody (Invitrogen) in blocking buffer was added to detect scFv and incubated at room temperature for 1 hour. After washing, HRP-goat anti-mouse antibody (Jackson ImmunoResearch) was added and incubated at room temperature for 1 hour. The plates were then washed three times with PBST and 3 times with PBS before HRP activity was detected with TMB 1-component Microwell Peroxidase Substrate (Seracare) and neutralized with 2N sulfuric acid.

For negative peptide-HLA complex counter-screening, scFv PPE ELISAs were performed as described above, except for the coating antigen. HLA mini-pools consisted of 2 ug/ml of each of the three negative peptide-HLA complexes pooled together and coated onto streptavidin plates for comparison binding to their particular peptide-HLA complex. HLA big pools consisted of 2 ug/ml of each of all 18 negative peptide-HLA complexes pooled together and coated onto streptavidin plates for comparison binding to their particular peptide-HLA complex.

Those scFvs that showed selectivity for target pHLA compared to negative control pHLA by scFv-ELISA as crude PPE, were separately expressed and purified. The purified scFvs were titratated by scFv ELISA for confirmation of binding only target pHLA compared to negative control pHLA (“Selective Binders”).

Clones were formatted into IgG, Fab, or scFv for further biochemical and functional analysis. ScFv clones selected for Fab production to be used for crystallization with their corresponding pHLA complexes were selected based on several parameters: sequence diversity, binding affinity, selectivity, and CDR3 diversity. The clustal software was used to produce a dendrogram and assess the sequence diversity of the Fab clones. The canonical 3D structures of the scFv sequences, based on the VH type, were also considered when possible. Binding affinity, as determined by the equilibrium dissociation constant (KD), was measured using an Octet HTX (ForteBio). Selectivity for the specific peptide-HLA complexes was determined with an ELISA titration of the purified scFvs and compared to negative peptides or streptavidin alone. Cutoff values for the KD and selectivity were determined for each target set based on the range of values obtained for the Fabs within each set. Final clones were then selected to obtain the highest diversity in sequence families and CDR3.

Table 2 shows the hit rate for the screening campaign described above.

TABLE 2
hit rate for screening campaigns

| Group | G2 | G7 |
|---|---|---|
| Gene target | CT83 | CT83 |
| HLA | A*01:01 | A*02:01 |
| Restricted peptide | NTDNNLAVY | LLASSILCA |
| # Sequence Unique Binders | 74 | 8 |
| # Selective Binders | 27 | 6 |
| # selected for IgG | 20 | 8 |
| # selected for Fab | 6 | 3 |
| # selected for scFv | 20 | 7 |

Table 3 shows the VH and VL sequences of the G2 scFv Selective Binders, selective for HLA-PEPTIDE Target HLA-A*01:01_NTDNNLAVY

Table 4 shows the CDR sequences for the G2 Selective Binders, selective for HLA-PEPTIDE Target HLA-A*01:01_NTDNNLAVY. CDRs were determined according to the Kabat numbering system.

Table 5 shows the VH and VL sequences of the G7 scFv Selective Binders, selective for HLA-PEPTIDE Target HLA-A*02:01_LLASSILCA.

Table 6 shows the CDR sequences for the G7 Selective Binders, selective for HLA-PEPTIDE Target HLA-A*02:01_LLASSILCA. CDRs were determined according to the Kabat numbering system.

### Example 7: Reformatting of Antibodies into Fab/scFv/IgG Clones

Selected clones were reformatted into Fab, scFv, or IgG formats as follows.

Construction and Production of Fab Protein Fragments

The constructs of selected G2, and G7 Fabs were cloned into a vector optimized for mammalian expression. Each DNA construct was scaled up for transfection and sequences were confirmed. A 100 mL transient production was completed in HEK293 cells (Tuna293™ Process) for each. The proteins were purified by anti-CH1 purification subsequently purified by SEC-polishing via HiLoad 16/600 Superdex 200. The mobile phase used for SEC-polishing was 20 mM Tris, 50 mM NaCl, pH 7. Final confirmatory CE-SDS analysis was performed.

Construction and Production of scFv Protein Fragments

The expression plasmid was transformed into BL21(DE3) strain and co-expressed with a periplasmid chaperone in a 400 mL E. coli culture. The cell pellet was reconstituted: 10 ml/1 g biomass with (25 mM HEPES, pH7.4, 0.3M NaCl, 10 mM MgCl2, 10% glycerol, 0.75% CHAPS, 1 mM DTT) plus lysozyme, and benzonase and Lake Pharma protease inhibitor cocktail. The cell suspension was incubated on a shaking platform at RT for 30 minutes. Lysates were clarified by centrifugation at 4C, 13,000×rpm for 15 min. The clarified lysate was loaded onto 5 ml of Ni NTA resin pre-equilibrated in IMAC Buffer A (20 mM Tris-HCl, Ph7.5; 300 mM NaCl/10% Glycerol/1 mM DTT). The resin was washed with 10CVs of Buffer A (or until a stable baseline was reached), followed by 10 CVs of 8% IMAC Buffer B (20 mM Tris-HCl, Ph7.5; 300 mM NaCl/10% Glycerol/1 mM DTT/250 mM Imidazole). The target protein was eluted in a 20CV gradient to 100% IMAC Buffer B. The column was washed with 5CVs of 100% IMAC B to ensure complete protein removal. Elution fractions were analyzed by SDS-PAGE and Western blot (anti-His) and pooled accordingly. The pool was dialyzed to versus final formulation buffer (20 mM Tris-HCl, Ph7.5; 300 mM NaCl/10% glycerol/1 mM DTT), concentrated to a final protein concentration >0.3 mg/ml, aliquoted into 1 mL vials, and flash frozen in liquid nitrogen. Final QC steps included SDS-PAGE and measuring A280.

Construction and Production of IgG Proteins

The expression constructs of the G series antibodies were cloned into a vector optimized for mammalian expression. Each DNA construct was scaled up for transfection and sequences were confirmed. A 10 mL transient production was completed in HEK293 cells (Tuna293™ Process) for each. The proteins were purified by Protein A purification and final CE-SDS analysis was performed.

### Example 8: Affinity of Fab Clones for the HLA-PEPTIDE Target

Affinity measurements were performed on the Octet Qke (ForteBio). Biotinylated pHLA complexes in 1× kinetics buffer were loaded onto streptavidin sensors at concentrations that gave the optimal nm shift response (approximately 0.6 nm) for each Fab at the highest concentration used. The pHLA complexes were loaded for 300 seconds and the ligand-loaded tips were subsequently equilibrated in the kinetics buffer for 120 seconds. The ligand-loaded biosensors were then dipped for 200 seconds in the Fab solution titrated into 2-fold dilutions. Starting Fab concentrations ranged from 100 nM to 2 uM, then optimized based on the K_{D} values of the Fab. The dissociation step in the kinetics buffer was measured for 200 seconds. Data was analyzed using the ForteBio data analysis software using a 1:1 binding model.

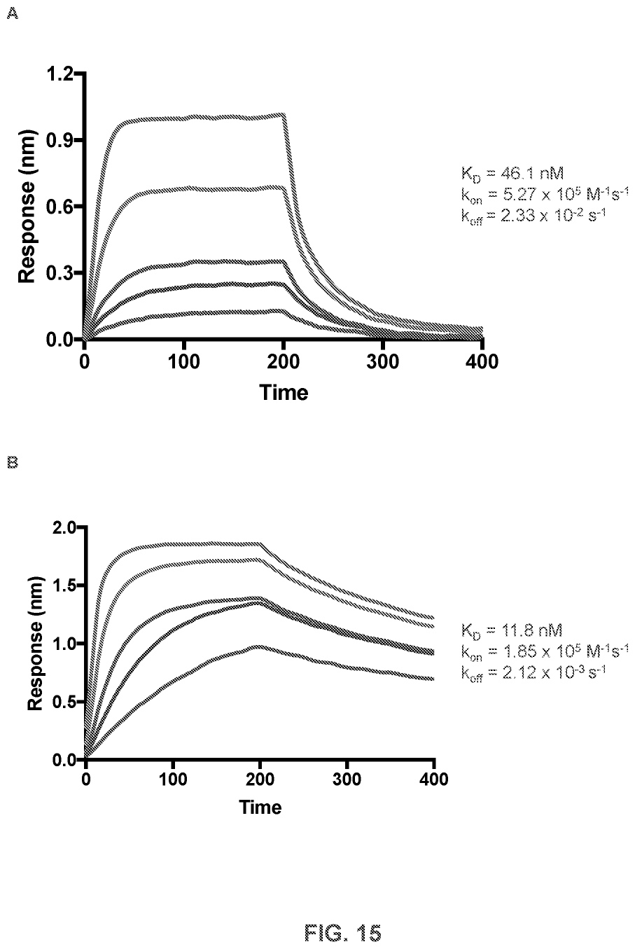

FIGS. 15A and 15B show BLI results for G2 target Fab clone G-2P1H11 and for G7 target Fab clone G7R4-B5-P2E9, respectively.

Results are shown in the Table below.

TABLE 7
Optimized Octet BLI affinity measurements of Fabs
binding to their target peptide-HLA complex

| Target | Fab clone | KD (M) | Kon (1/Ms) | Kdis (1/s) | Full R\hat{ }2 |
|---|---|---|---|---|---|
| G2 | G2-P1B06 | 4.44E−08 | 1.06E+06 | 3.23E−02 | 0.991 |
| G2 | G2-P2A03 | 1.09E−07 | 3.32E+05 | 3.60E−02 | 0.998 |
| G2 | G2-P1B12 | 2.28E−08 | 3.66E+05 | 7.28E−03 | 0.980 |
| G2 | G2-P2A11 | 2.81E−08 | 6.33E+05 | 1.72E−02 | 0.992 |
| G2 | G2-P1H01 | 1.55E−08 | 9.52E+05 | 1.48E−02 | 0.984 |
| G2 | G2-P1H11 | 4.99E−08 | 5.81E+05 | 2.80E−02 | 0.994 |
| G7 | 2-G7R4-P2C2 | 5.31E−07 | 1.04E+05 | 5.43E−02 | 0.986 |
| G7 | 3-G7R4-P1A3 | 5.32E−07 | 1.97E+05 | 9.94E−02 | 0.988 |
| G7 | 4-G7R4-B5- | 1.18E−08 | 1.85E+05 | 2.12E−03 | 0.992 |
| | P2E9 | | | | |

### Example 9: Positional Scanning of G2 and G7 Restricted Peptide Sequences

Positional scanning of the G2 and G7 restricted peptides was carried out to determine the amino acid residues which act as contact points for selected Fab clones.

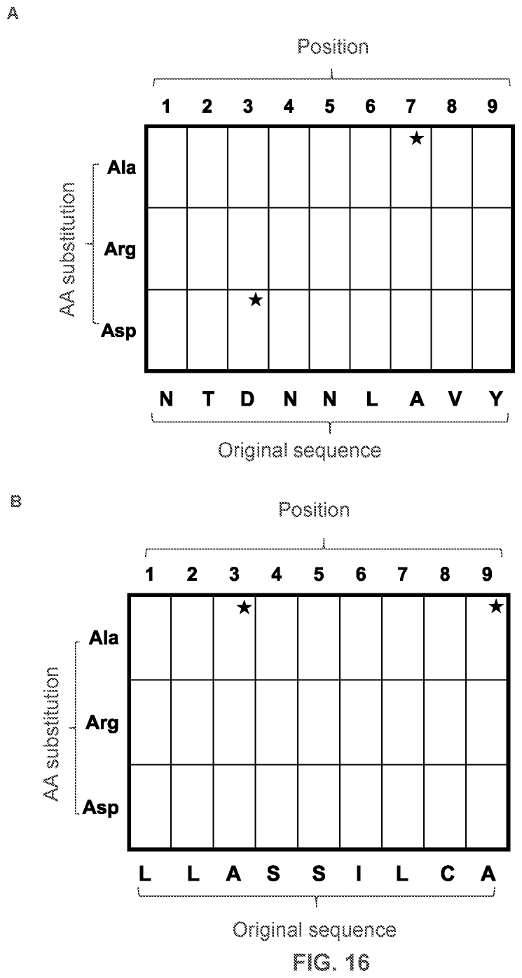

Briefly, positional scanning libraries of variant G2 and G7 restricted peptides were generated with amino acid substitutions at a single position in the G2 or G7 peptide sequence, scanning across all positions. The amino acid substitutions at a given position were either alanine (conservative substitution), arginine (positively charged), or aspartate (negatively charged). A map of the amino acid substitutions for the positional scanning experiment is shown in FIG. 16. Asterisks denote lack of amino acid substitution.

Peptide-HLA complexes comprising the positional scanning library members and the HLA subtype allele were generated as described in Example 6. To determine whether the variant G2 and G7 peptides could complex with the desired HLA alleles, stability analyses of the resulting complexes were carried out using conditional ligand peptide exchange and ELISA as described in Example 6. Next, binding affinity of the positional variant-HLA complexes to the Fab clones was assessed by BLI, as described in Example 8.

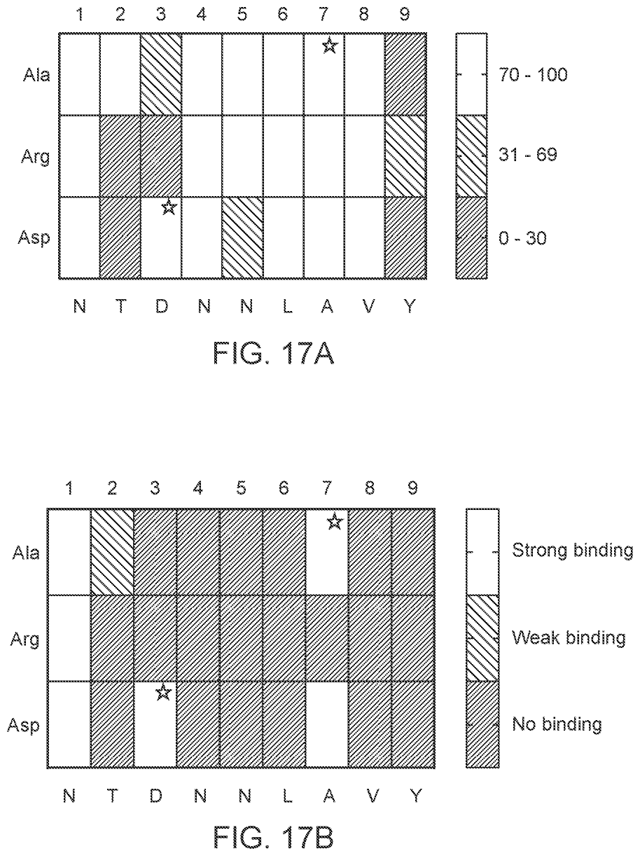

A stability heat map for the G2 positional variant-HLAs is shown in FIG. 17A. [Red] denotes very low stability, [gray] denotes low stability, and [blue] denotes high stability. FIG. 17A shows that the C-terminal amino acid residue (position 9) and the second and third N-terminal residues (positions 2 and 3) were critical residues for anchoring the peptide to the HLA thereby stabilizing the ternary complex.

An affinity heat map for Fab clone G2-P1H11 is shown in FIG. 17B. The degree of binding indicated on the heatmap is based on the nm shift on the BLI biosensor due to Fab binding to the pHLA. As stated above, [Red] denotes no binding affinity (−0.02 to 0.18 nm shift), [gray] denotes weak binding affinity (0.19-0.25 nm shift), and [blue] denotes high binding affinity (0.26-0.32 nm shift). As expected, positional mutations which resulted in unstable complexes (at positions 2, 3, and 9) also resulted in no Fab binding. FIG. 17B shows that introduced substitutions at positions 3-8 resulted in failure of the Fab clone to bind the HLA-peptide complex. These results suggest that the majority of the residues which are not involved in binding the HLA molecule, and are residues that likely protrude from the HLA protein, are important for peptide-specificity of Fab clone G2-P1H11.

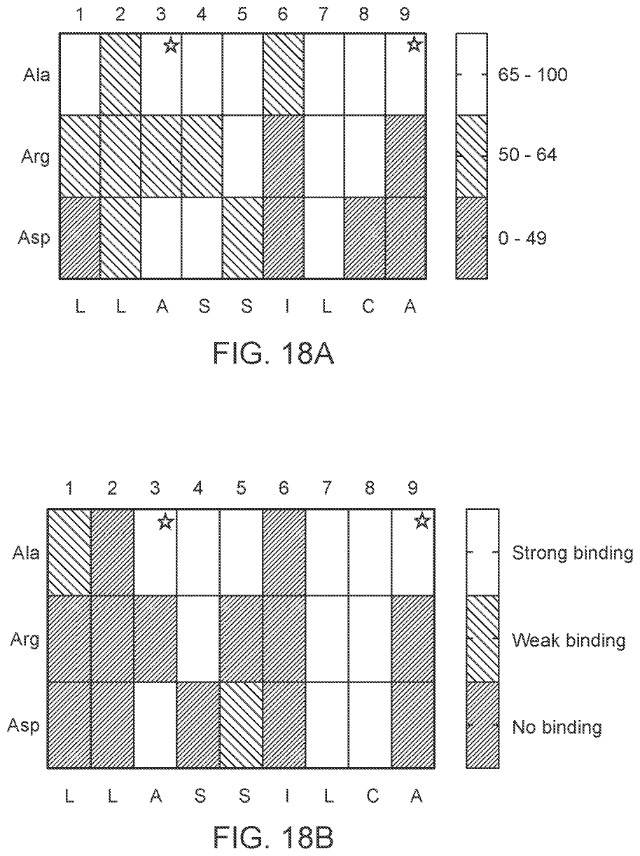

A stability heat map for the G7 positional variants is shown in FIG. 18A. Positions 1, 2, 6, and 9 appear to be important for stabilizing the HLA complex.

An affinity heat map for Fab clone G7R4-B5-P2E9 is shown in FIG. 18B. As stated above, [Red] denotes no binding affinity (−0.02 to 0.18 nm shift), [gray] denotes weak binding affinity (0.19-0.25 nm shift), and [blue] denotes high binding affinity (0.26-0.72 nm shift). indicating that positions 1-5 are important for peptide-specificity of the Fab clone.

### Example 10: Generated Antibodies Successfully Bind Cells Presenting the HLA-PEPTIDE Target

IgGs from scFv clones G2-P1H11 and G7-Ep were created as described in Example 7.

The ability of IgGs to bind to K562 cells pulsed with the target restricted peptide was assessed by flow cytometry.

Retroviral Production

Phoenix-AMPHO cells (ATCC®, CRL-3213™) were plated at 5×10⁵ cells/well in a 6 well plate and incubated overnight at 37° C.

Phoenix-AMPHO cells were transfected with retroviral vectors containing expression cassettes for the desired HLA subtypes as follows. 10 g plasmid, 10 μL Lipofectamine LTX PLUS (Fisher Scientific, cat #15338100) reagent and 100 μL Opti-MEM (Gibco™, cat #31985062) were incubated at room temperature for 15 minutes. Simultaneously, 8 μL Lipofectamine was incubated with 92 μL Opti-MEM at room temperature for 15 minutes. These two reactions were combined and incubated again for 15 minutes at room temperature after which 800 μL Opti-MEM was added. The culture media was aspirated from the Phoenix cells and they were washed with 5 mL pre-warmed Opti-MEM. The Opti-MEM was aspirated from the cells and the lipofectamine mixture was added. The cells were incubated for 3 hours at 37° C. and 3 mL complete culture medium was added. The plate was then incubated overnight at 37° C. The media was replaced with Phoenix culture medium and the plate incubated an additional 2 days at 37° C.

The media was collected and filtered through a 45 μm filter into a clean 6 well dish. 20 μL Plus reagent was added to each virus suspension and incubated at room temperature for 15 minutes followed by the addition of 8 μL/well of Lipofectamine and another 15 minute room temperature incubation.

Generation of K562 Cells Expressing the HLA-PEPTIDE Targets and Cell Binding with Exemplary IgG Clones

K562 cells, which lack endogenous MHC, were transduced with retrovirus for introduction of the HLA subtype for G2 or G7, respectively. HLA-transduced K562 cells were pulsed the night before with 50 μM of target or negative control peptide (Genscript) in IDMEM containing 1% FBS in 6 well plates and incubated under standard tissue culture conditions. Cells were harvested, washed in PBS, and stained with eBioscience Fixable Viability Dye eFluor 450 for 15 minutes at room temperature. Following another wash in PBS+1% FBS, cells were resuspended with test IgGs (G2-P1H11 or G7R4-B5-P2E9) at varying concentrations. Cells were incubated with the antibodies for 1 hour at 4° C. After another wash, PE-congugated goat anti-human IgG secondary antibody (Jackson ImmunoResearch) was added at 1:200 for 30 minutes at 4° C. After washing in PBS+1% FBS, cells were resuspended in PBS+1% FBS and analyzed by flow cytometry. Flow cytometric analysis was performed on the Attune NxT Flow Cytometer (ThermoFisher) using the Attune NxT Software. Data was analyzed using FlowJo.

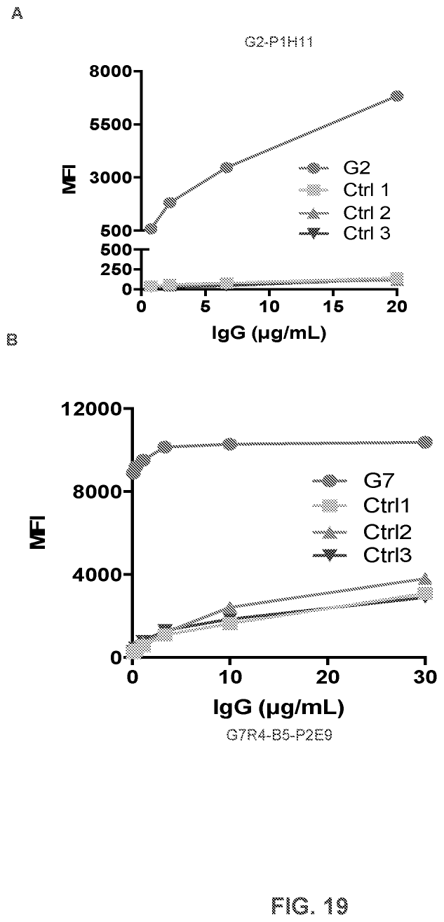

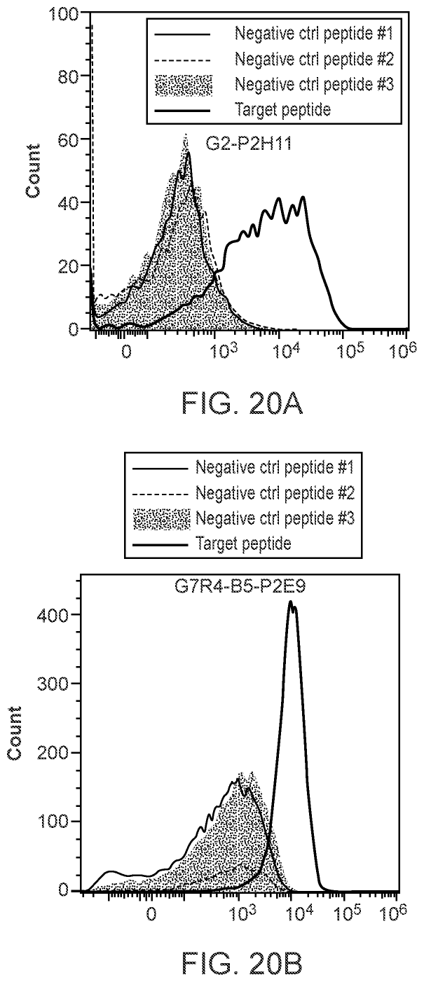

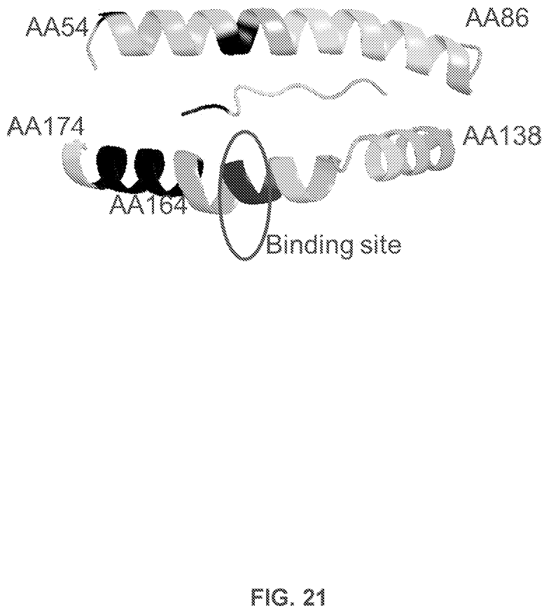

Results are shown in FIGS. 19 and 20. Both G2-P1H11 and G7R4-B5-P2E9 selectively bound HLA-transduced K562 cells pulsed with the target peptide, as compared to HLA-transduced cells pulsed with the negative control peptides.

In Vivo Proof-of-Concept

Lead antibody or CAR-T constructs are evaluated in vivo to demonstrate directed tumor killing in humanized mouse tumor models. Lead antibody or CAR-T constructs are evaluated in xenograft tumor models engrafted with human tumors and PBMCs. Anti-tumor activity is measured and compared to control constructs to demonstrate target-specific tumor killing.

### Example 11: scFv-pHLA Structures by Hydrogen/Deuterium Exchange and Mass Spectrometry

Hydrogen/Deuterium Exchange

20 μM of HLA-peptide was incubated with a 3-fold molar excess of scFv or Fab formatted ABPs for 20 min at room temperature (20-25° C.) to generate complexes for the exchange experiments. For the Apo control, the HLA-peptide was incubated with an equal volume of 50 mM NaCl, 20 mM Tris pH 8.0, without the ABP. All subsequent reaction steps were performed at 4° C. by an automated HDX PAL system controlled by Chronos 4.8.0 software (Leap Technologies, Morrisville, N.C.). Deuterium exchange was carried out in duplicate for time periods ranging from 30 s to 3 hrs. 5 μl of protein complexes were diluted 10-fold into H2O (for the 0 min. control time-point) or D20 for the indicated time-points prior to quenching in 0.8 M guanidine hydrochloride, 0.4% acetic acid (v/v), and 75 mM tris(2-carboxyethyl) phosphine for 3 min.˜50 pmol of quenched protein complexes were transferred onto an immobilized Protein XIII/Pepsin column (NovaBioAssays, Woburn, Mass.) for integrated on-line protein digestion.

Liquid Chromatography, Mass Spectrometry, and HDX Analysis

Chromatographic separation of peptides was carried out using an UltiMate 3000 Basic Manual UHPLC System (ThermoFisher Scientific, Waltham, Mass.), which contained a trap C18 column (5 μM particle size and 2.1 mm diameter) and an analytical C18 column (1.9 μM particle size and 1 mm diameter). Samples were desalted with 10% acetonitrile, 0.5% formic acid at a 40 μl/min flow rate for 2 min and peptides were eluted at a 40 μl/min flow rate with an increasing concentration of 95% acetonitrile, 0.5% formic acid. Mass spectrometry was performed with an Orbitrap Fusion Lumos mass spectrometer (ThermoFisher, Waltham, Mass.) with the ESI source set at a positive ion voltage of 3700 V. Prior to performing hydrogen-deuterium exchange experiments, peptide fragments of each HLA-peptide complex were analyzed by data-dependent LC/MS/MS and the data searched using PEAKS Studio (Bioinformatics Solutions Inc., Waterloo, ON, Canada) with a peptide precursor mass tolerance of 10 ppm and fragment ion mass tolerance of 0.1 Da. The sequences of the HLA, β2M, and the peptide were searched, and false detection rates identified using a decoy-database strategy. Peptides from the hydrogen-deuterium experiments were detected by LC/MS and analyzed by HDX Workbench (Omics Informatics, Honolulu, Hi.) with a retention time window size of 0.22 min and a 7.0 ppm error. In the deuterium (D) exchange samples the peptide was then identified in the elution time window and the difference in mass was determined. Mass increases by 1 for each D exchanged in the peptide backbone since D has a molecular weight of 1 more than H. The difference in mass and the intensity of the isotopic ions produced values of % D for each peptide. Differences in deuterium uptake were mapped to relevant protein crystallographic structures using Pymol (Schrödinger, Cambridge, Mass.). The decrease in D exchange between the Apo control and ABP was calculated and plotted by amino acid. Statistical analysis and graphical representations were performed using GraphPad Prism 7.0 (La Jolla, Calif.).

An example of the data from scFv G2-P1G07 plotted on a crystal structure PDB 5bs0 is shown in FIG. 21. The crystal structure can be found at URL https://www.rcsb.org/structure/5bs0 (Raman et al). Areas not covered with MS data are shown in black and those with the greatest decrease in D exchange (indicating a binding site for the ABP) is circled. For clarity, only the binding groove and helices are shown.

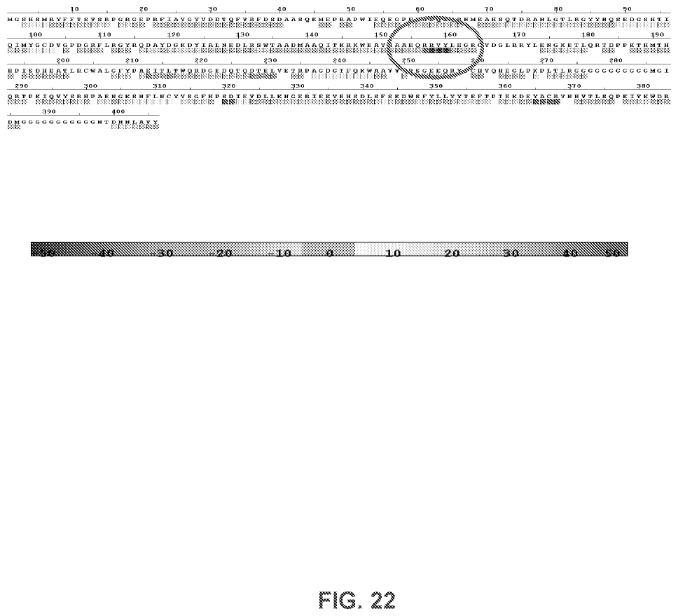

An exemplary heatmap for scFv clone G2-P1G07 visualized in its entirety using a consolidated perturbation view is shown in FIG. 22.

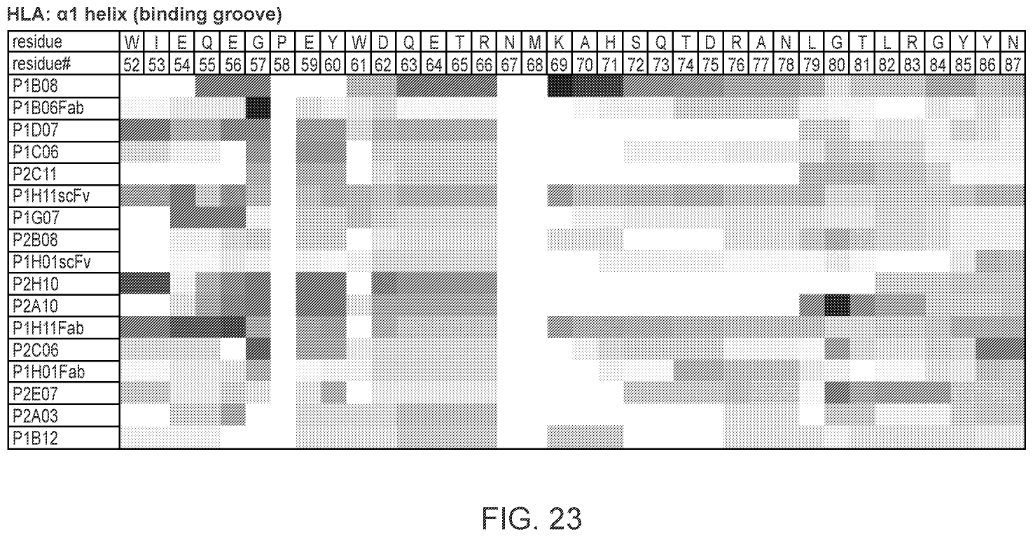

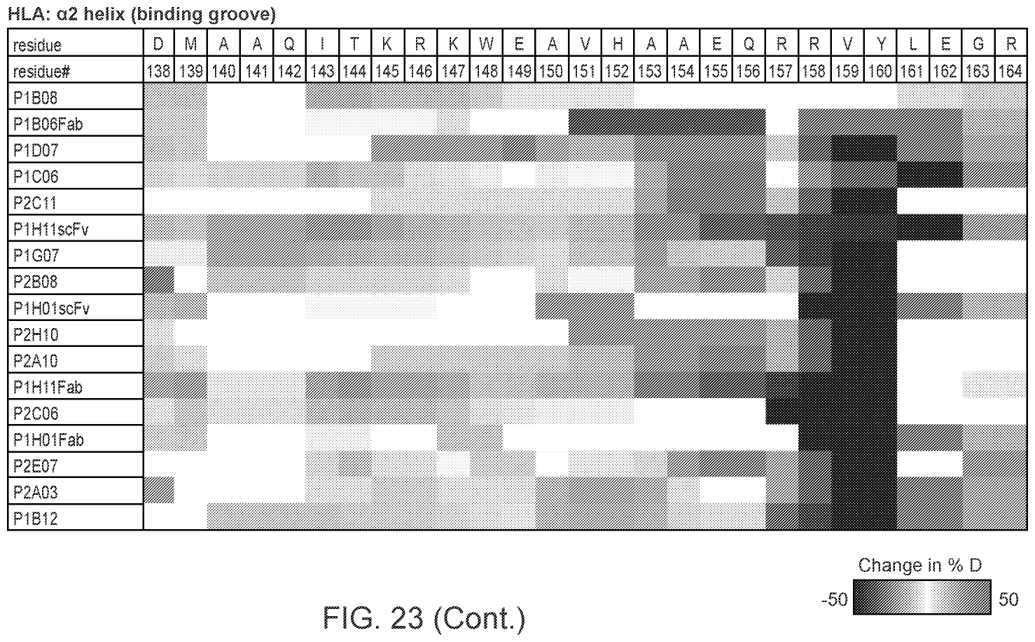

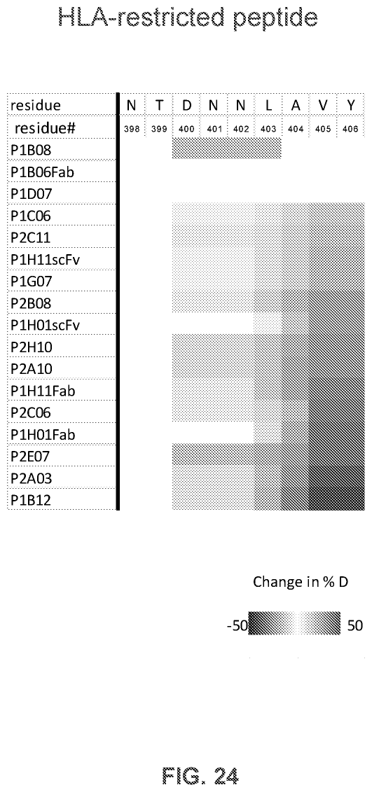

To better compare the data across the ABPs tested for a given HLA-PEPTIDE target, data for each ABP was exported, and a heat map was generated in Excel. Resulting heat maps are shown in FIG. 23 showing a heat map across the al helix (top) and across the α2 helix (bottom). FIG. 24 shows a heat map for all ABPs tested for A*0101_NTDNNLAVY, across restricted peptide residues 1-9. These results indicate that residues 6-9 of the restricted peptide, and HLA residues 157-160 are important contact points of the A*0101_NTDNNLAVY HLA-PEPTIDE target complex for binding to its specific ABP. All clone entries in the HDX heat maps are scFv formats unless otherwise noted.

### Example 12: Isolation of TCRs that Specifically Bind HLA-PEPTIDE Targets

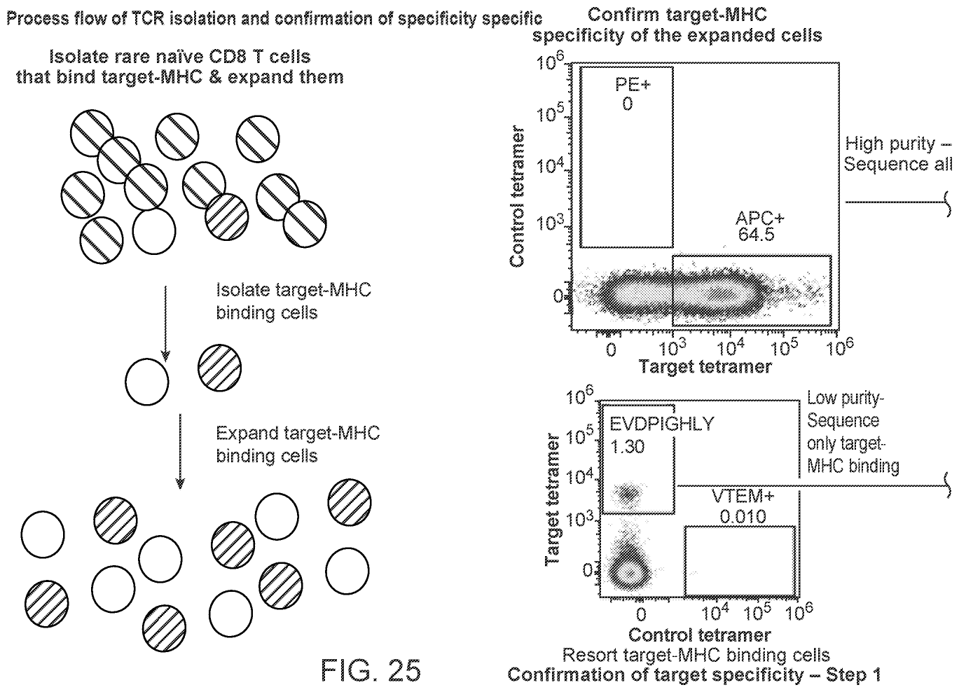

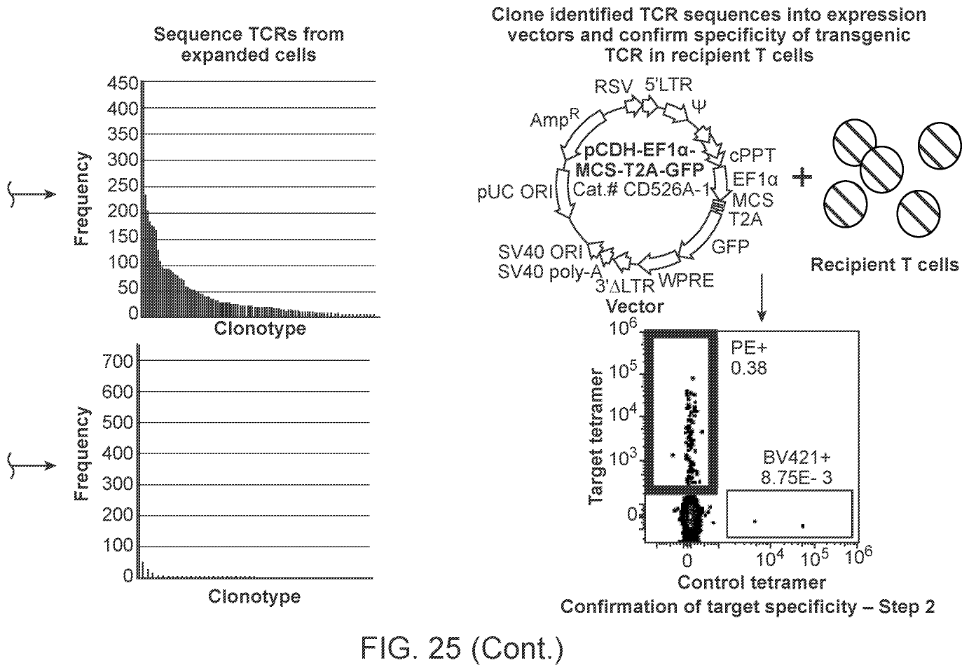

FIG. 25 depicts an experimental workflow by which TCRs which specifically bind HLA-PEPTIDE targets were isolated. Briefly, naïve CD8+ T cells that bind to the HLA-PEPTIDE target were isolated by flow cytometry and polyclonally expanded. Following expansion, specificity of cells for HLA-PEPTIDE target complex was tested by flow cytometry. If a large fraction (>75%) of an expanded population was specific for the HLA-PEPTIDE target, the population as a whole was sequenced as a whole to identify TCRs. Alternatively, cells that specifically bound the HLA-PEPTIDE target were resorted, and only cells isolated after resort were sequenced. TCR sequences were cloned into expression vectors and introduced into recipient T cells as recombinant TCRs. Expression of the evaluated TCR and binding of cognate HLA-PEPTIDE target complex by the TCR-recombinant T cells was assessed.

Identified HLA-PEPTIDE Targets were Readily Recognized by CD8+ T Cells

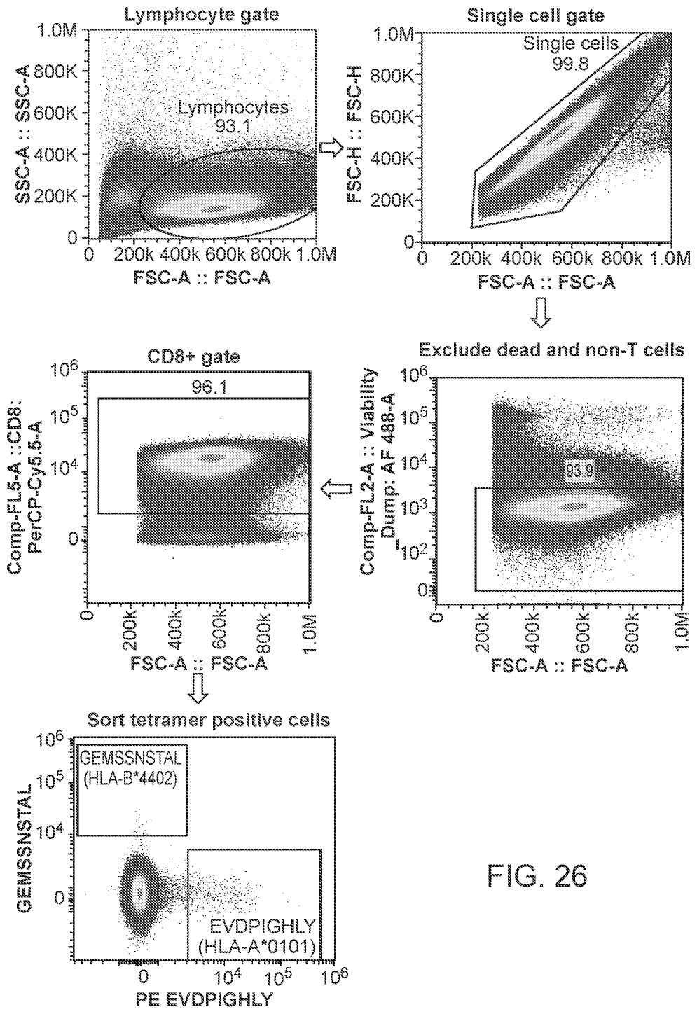

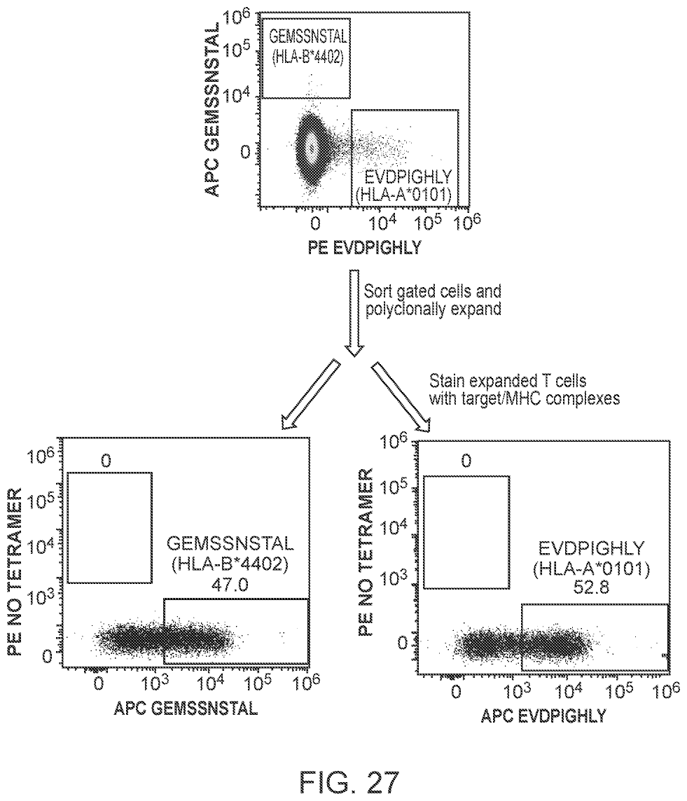

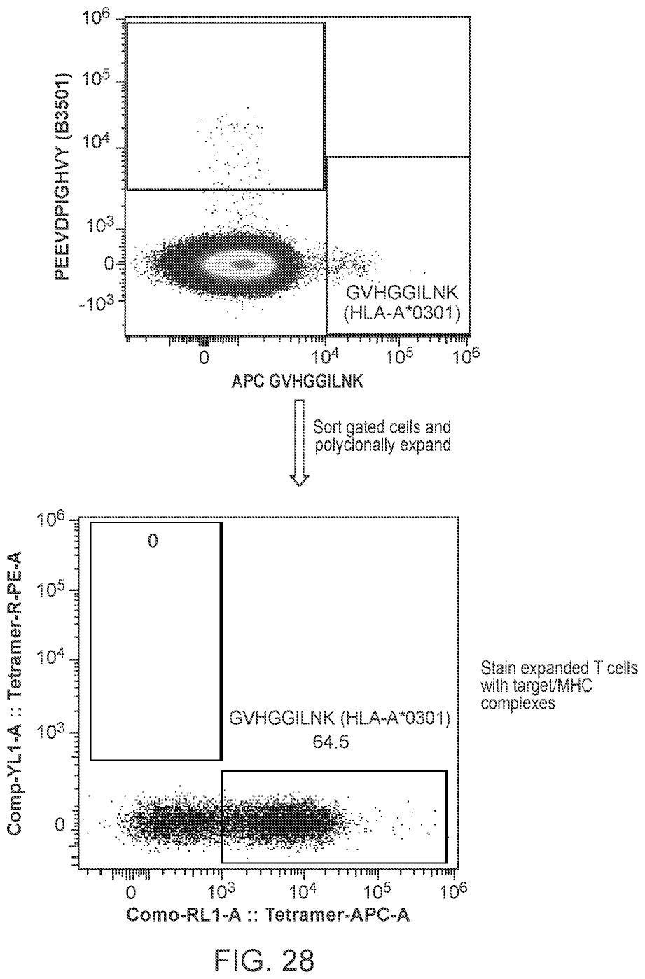

Peripheral Blood Mononuclear Cells (PBMCs) from healthy donors were magnetically enriched for naïve CD8+ T cells as follows. PBMCs were obtained by processing leukapheresis samples from healthy donors. Frozen PBMCs were thawed and incubated with cocktail of biotinylated CD45RO, CD14, CD15, CD16, CD19, CD25, CD34, CD36, CD57, CD123, anti-HLA-DR, CD235a (Glycophorin A), CD244, and CD4 antibodies and were subsequently magnetically labeled with anti-biotin microbeads for removal from PBMC population. Enriched naïve CD8 T cells were labelled with tetramers comprising of target peptide and appropriate HLA molecule, stained with live/dead and lineage markers and sorted by flow cytometry according to the gating procedure depicted in FIG. 26. Cells that bound the HLA-PEPTIDE tetramers were isolated. Following polyclonal expansion, specificity of expanded CD8+ T cells was reassessed by labeling with the HLA-PEPTIDE or no tetramer control. Flow cytometry results for exemplary HLA-PEPTIDE targets B*44:02 GEMSSNSTAL and A*01:01 EVDPIGHLY are shown in FIG. 27. Flow cytometry results for the HLA-PETPIDE target A*03:01_GVHGGILNK is shown in FIG. 28.

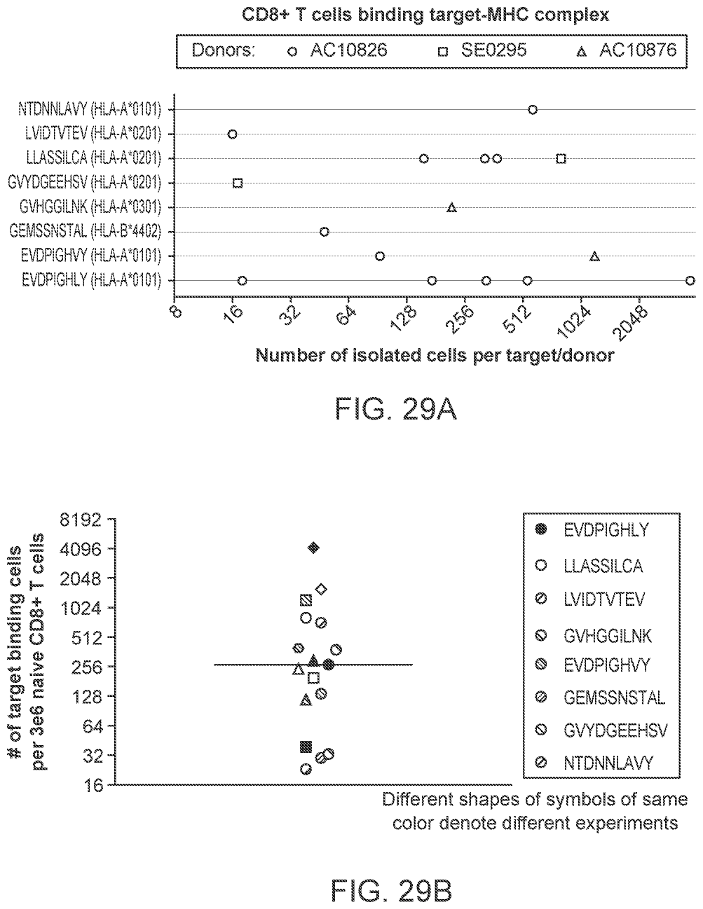

The number of isolated CD8+ T cells per HLA-PEPTIDE target per donor and distribution of isolated CD8+ T cells frequency per HLA-PEPTIDE target across all donors tested is shown in FIG. 29: 29A (number of isolated CD8+ T cells) and 29B (frequency). Total number of isolated naïve CD8+ T cells per target ranged from 23-4181 antigen specific cells, which is in line with precursor frequencies of T cells specific for known immunogenic viral antigens. These cells present the source of natural TCRs for sequencing and further characterization.

The number of isolated target-specific T cells per target summarized across all tested donors is shown in Table 8.

TABLE 8
number of isolated target-specific T cells
per target summarized across all donors

| | | Cumulative Number |
|---|---|---|
| | | of TCR Source |
| Target | Gene | Cells Per Target |
| EVDPIGHLY | | |
| (HLA-A*0101) | MAGEA3 | 5242 |
| EVDPIGHVY | | |
| (HLA-A*0101 | MAGEA6 | 1296 |
| GEMSSNSTAL | | |
| (HLA-B*4402) | CT 83 | 48 |
| GVHGGILNK | | |
| (HLA-A*0301) | PFN3 | 219 |
| GVYDGEEHSV | | |
| (HLA-A*0201) | MAGEB2 | 17 |
| LLASSILCA | | |
| (HLA-A*0201) | CT 83 | 1665 |
| LVIDTVTEV | | |
| (HLA-A*0201) | SPERT | 16 |
| NTDNNLAVY | | |
| (HLA-A*0101) | CT 83 | 575 |

These data demonstrate that identified HLA-PEPTIDE targets are biologically relevant, as natural CD8+ T cells exist in HLA matched human blood which bind/recognize target peptides in the context of predicted associated MHC molecule.

CD8+ T Cells Yielded a Diverse Repertoire of Unique TCRs which Bound the HLA-PEPTIDE Targets

Criteria for Sequencing of T-Cells

If a large fraction (>75%) of an expanded population was specific for the HLA-PEPTIDE target, the population as a whole was sequenced as a whole to identify TCRs. Then, selected TCR sequences from the population were cloned into expression vectors and transfected into recipient T-cells for confirmation of specificity. Alternatively, cells that specifically bound the HLA-PEPTIDE target were resorted, and only cells isolated after resort were sequenced.

Sequencing Protocol

T cells isolated and expanded as described in FIG. 26 were sequenced using 10× Genomics single cell resolution paired immune TCR profiling approach. Specifically, two-to-eight thousand live T cells were partitioned into single cell emulsions for subsequent single cell cDNA generation and full-length TCR profiling (5′ UTR through constant region ensuring alpha and beta pairing). One approach utilizes a molecularly barcoded template switching oligo at the 5′end of the transcript, a second approach utilizes a molecularly barcoded constant region oligo at the 3′ end, and a third approach couples an RNA polymerase promoter to either the 5′ or 3′ end of a TCR. All of these approaches enable the identification and deconvolution of alpha and beta TCR pairs at the single-cell level. The resulting barcoded cDNA transcripts underwent an optimized enzymatic and library construction workflow to reduce bias and ensure accurate representation of clonotypes within the pool of cells. Libraries were sequenced on Illumina's MiSeq or HiSeq4000 instruments (paired-end 150 cycles) for a target sequencing depth of about five to fifty thousand reads per cell.

Sequencing reads were processed through the 10× provided software Cell Ranger. Sequencing reads were tagged with a Chromium cellular barcodes and UMIs, which were used to assemble the V(D)J transcripts cell by cell. The assembled contigs for each cell were then annotated by mapping the assembled contigs to V(D)J reference sequences from Ensembl version 87 (http://www.ensembl.org/).

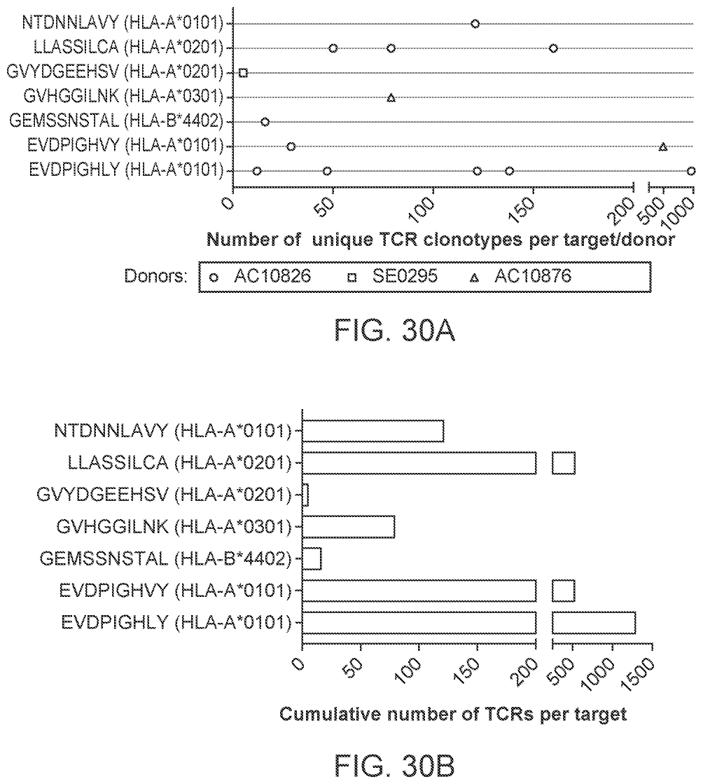

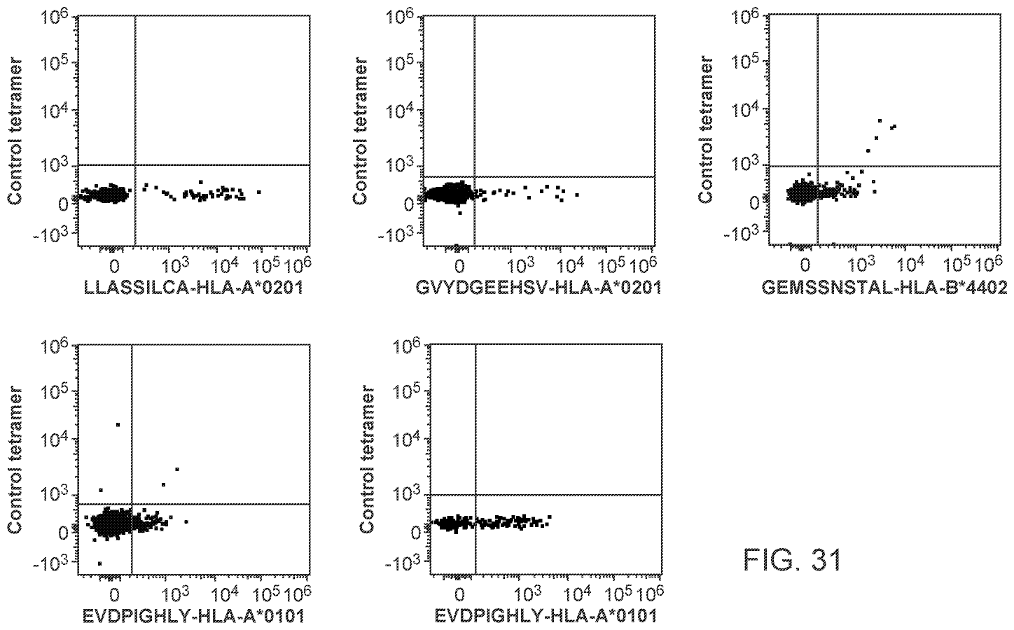

Clonotypes were defined as alpha, beta chain pairs of unique CDR3 amino acid sequences. Clonotypes were filtered for single alpha and single beta chain pairs present at frequency above 2 cells to yield the final list of clonotypes per target peptide in a specific donor. FIG. 30A depicts the number of unique TCR clonotypes per HLA-PEPTIDE target for each tested donor. FIG. 30B depicts the total number of unique clonotypes per HLA-PEPTIDE target, summed across all donors tested.

TCR Sequences of Unique Clonotypes from Resorted Cells

Annotated variable, diversity, joining, and constant regions of TCR clonotypes specific for A*0101_EVDPHIGHLY, from resorted cells, are shown below in Table 9.

TABLE 9
annotated TCR sequences of unique TCRs specific for
A*0101_EVDPHIGHLY, sequenced from resorted cells

| TCR ID # | PEPTIDE | HLA | TRW | TRAJ | TRAC | TRBV | TRBD | TRBJ | TRBC |
|---|---|---|---|---|---|---|---|---|---|
| TCR101 | EVDPIGHLY | A0101 | TRAV12-3 | TRAJ20 | TRAC | TRBV20-1 | TRBD2 | TRBJ1-2 | TRBC1 |
| TCR102 | EVDPIGHLY | A0101 | TRAV19 | TRAJ40 | TRAC | TRBV20-1 | TRBD1 | TRBJ2-7 | TRBC2 |
| TCR103 | EVDPIGHLY | A0101 | TRAV21 | TRAJ4 | TRAC | TRBV10-3 | None | TRBJ1-1 | TRBC1 |
| TCR104 | EVDPIGHLY | A0101 | TRAV12-3 | TRAJ20 | TRAC | TRBV20-1 | TRBD1 | TRBJ2-7 | TRBC2 |
| TCR105 | EVDPIGHLY | A0101 | TRAV1-1 | TRAJ4 | TRAC | TRBV9 | TRBD1 | TRBJ1-1 | TRBC1 |
| TCR106 | EVDPIGHLY | A0101 | TRAV12-1 | TRAJ17 | TRAC | TRBV6-1 | TRBD2 | TRBJ2-1 | TRBC2 |
| TCR107 | EVDPIGHLY | A0101 | TRAV4 | TRAJ47 | TRAC | TRBV20-1 | TRBD2 | TRBJ2-3 | TRBC2 |
| TCR108 | EVDPIGHLY | A0101 | TRAV21 | TRAJ6 | TRAC | TRBV5-4 | None | TRBJ2-1 | TRBC1 |
| TCR109 | EVDPIGHLY | A0101 | TRAV12-1 | TRAJ11 | TRAC | TRBV11-3 | TRBD1 | TRBJ1-1 | TRBC1 |
| TCR110 | EVDPIGHLY | A0101 | TRAV21 | TRAJ31 | TRAC | TRBV5-1 | TRBD1 | TRBJ1-1 | TRBC1 |
| TCR111 | EVDPIGHLY | A0101 | TRAV21 | TRAJ33 | TRAC | TRBV5-1 | TRBD1 | TRBJ2-3 | TRBC2 |
| TCR112 | EVDPIGHLY | A0101 | TRAV34 | TRAJ40 | TRAC | TRBV9 | TRBD2 | TRBJ2-7 | TRBC2 |
| TCR113 | EVDPIGHLY | A0101 | TRAV29DV5 | TRAJ29 | TRAC | TRBV7-9 | TRBD1 | TRBJ2-3 | TRBC2 |
| TCR114 | EVDPIGHLY | A0101 | TRAV19 | TRAJ40 | TRAC | TRBV20-1 | TRBD2 | TRBJ1-2 | TRBC1 |
| TCR115 | EVDPIGHLY | A0101 | TRAV4 | TRAJ47 | TRAC | TRBV20-1 | TRBD1 | TRBJ2-7 | TRBC2 |
| TCR116 | EVDPIGHLY | A0101 | TRAV21 | TRAJ54 | TRAC | TRBV5-1 | TRBD1 | TRBJ2-1 | TRBC2 |
| TCR117 | EVDPIGHLY | A0101 | TRAV21 | TRAJ42 | TRAC | TRBV7-9 | TRBD1 | TRBJ2-7 | TRBC2 |
| TCR118 | EVDPIGHLY | A0101 | TRAV21 | TRAJ4 | TRAC | TRBV20-1 | TRBD1 | TRBJ2-7 | TRBC2 |
| TCR119 | EVDPIGHLY | A0101 | TRAV21 | TRAJ40 | TRAC | TRBV29-1 | None | TRBJ2-2 | TRBC2 |
| TCR120 | EVDPIGHLY | A0101 | TRAV29DV5 | TRAJ49 | None | TRBV10-2 | TRBD1 | TRBJ2-7 | TRBC2 |
| TCR121 | EVDPIGHLY | A0101 | TRAV21 | TRAJ40 | TRAC | TRBV27 | TRBD2 | TRBJ2-2 | TRBC2 |
| TCR122 | EVDPIGHLY | A0101 | TRAV21 | TRAJ11 | TRAC | TRBV5-4 | None | TRBJ2-2 | TRBC1 |
| TCR123 | EVDPIGHLY | A0101 | TRAV12-3 | TRAJ20 | TRAC | TRBV20-1 | TRBD2 | TRBJ2-3 | TRBC2 |
| TCR124 | EVDPIGHLY | A0101 | TRAV26-2 | TRAJ49 | TRAC | TRBV19 | None | TRBJ1-5 | TRBC1 |
| TCR125 | EVDPIGHLY | A0101 | TRAV12-3 | TRAJ20 | TRAC | TRBV6-1 | TRBD2 | TRBJ2-1 | TRBC2 |
| TCR126 | EVDPIGHLY | A0101 | TRAV17 | TRAJ34 | TRAC | TRBV11-1 | TRBD1 | TRBJ1-2 | TRBC1 |
| TCR127 | EVDPIGHLY | A0101 | TRAV12-3 | TRAJ20 | TRAC | TRBV10-3 | None | TRBJ1-1 | TRBC1 |
| TCR128 | EVDPIGHLY | A0101 | TRAV21 | TRAJ26 | TRAC | TRBV5-6 | TRBD1 | TRBJ2-7 | TRBC2 |
| TCR129 | EVDPIGHLY | A0101 | TRAV29DV5 | TRAJ4 | TRAC | TRBV27 | TRBD1 | TRBJI-5 | TRBC1 |
| TCR130 | EVDPIGHLY | A0101 | TRAV4 | TRAJ47 | TRAC | TRBV20-1 | TRBD2 | TRBJ1-2 | TRBC1 |
| TCR131 | EVDPIGHLY | A0101 | TRAV13-1 | TRAJ49 | TRAC | TRBV27 | TRBD1 | TRBJ2-7 | TRBC2 |
| TCR132 | EVDPIGHLY | A0101 | TRAV12-1 | TRAJ10 | TRAC | TRBV25-1 | TRBD1 | TRBJ2-7 | TRBC2 |
| TCR133 | EVDPIGHLY | A0101 | TRAV29DV5 | TRAJ39 | TRAC | TRBV7-9 | None | TRBJ2-7 | TRBC2 |
| TCR134 | EVDPIGHLY | A0101 | TRAV21 | TRAJ47 | TRAC | TRBV9 | TRBD1 | TRBJ1-1 | TRBC1 |
| TCR135 | EVDPIGHLY | A0101 | TRAV39 | TRAJ41 | TRAC | TRBV13 | None | TRBJ1-4 | TRBC1 |
| TCR136 | EVDPIGHLY | A0101 | TRAV17 | TRAJ53 | TRAC | TRBV29-1 | TRBD1 | TRBJ2-1 | TRBC2 |
| TCR137 | EVDPIGHLY | A0101 | TRAV26-1 | TRAJ42 | TRAC | TRBVI9 | TRBD1 | TRBJ2-3 | TRBC2 |
| TCR138 | EVDPIGHLY | A0101 | TRAV8-6 | TRAJ50 | TRAC | TRBV9 | TRBD1 | TRBJ2-7 | TRBC2 |
| TCR139 | EVDPIGHLY | A0101 | TRAV19 | TRAJ10 | TRAC | TRBV7-9 | None | TRBJ2-7 | TRBC2 |
| TCR140 | EVDPIGHLY | A0101 | TRAV8-4 | TRAJ42 | TRAC | TRBV3-1 | TRBD2 | TRBJ2-1 | TRBC2 |
| TCR141 | EVDPIGHLY | A0101 | TRAV12-1 | TRAJ47 | TRAC | TRBV5-8 | TRBD1 | TRBJ1-1 | TRBC1 |
| TCR142 | EVDPIGHLY | A0101 | TRAV29DV5 | TRAJ42 | TRAC | TRBV10-3 | None | TRBJ2-7 | TRBC2 |
| TCR143 | EVDPIGHLY | A0101 | TRAV13-2 | TRAJ20 | TRAC | TRBV27 | TRBD2 | TRBJ1-1 | TRBC1 |
| TCR144 | EVDPIGHLY | A0101 | TRAV10 | TRAJ9 | TRAC | TRBV3-1 | TRBD1 | TRBJ1-3 | TRBC1 |
| TCR145 | EVDPIGHLY | A0101 | TRAV19 | TRAJ27 | TRAC | TRBV27 | TRBD1 | TRBJ2-7 | TRBC2 |
| TCR146 | EVDPIGHLY | A0101 | TRAV9-2 | TRAJ20 | TRAC | TRBV12-4 | TRBD1 | TRBJ2-1 | TRBC2 |
| TCR147 | EVDPIGHLY | A0101 | TRAV12-2 | TRAJ20 | TRAC | TRBV7-6 | TRBD2 | TRBJ2-1 | TRBC2 |
| TCR148 | EVDPIGHLY | A0101 | TRAV12-1 | TRAJ17 | TRAC | TRBV20-1 | TRBD2 | TRBJ1-2 | TRBC1 |
| TCR149 | EVDPIGHLY | A0101 | TRAV30 | TRAJ58 | TRAC | TRBV19 | None | TRBJ2-7 | TRBC2 |
| TCR150 | EVDPIGHLY | A0101 | TRAV8-1 | TRAJ43 | TRAC | TRBV7-8 | TRBD2 | TRBJ2-1 | TRBC2 |
| TCR151 | EVDPIGHLY | A0101 | TRAV13-1 | TRAJ9 | TRAC | TRBV9 | TRBD1 | TRBJ2-5 | TRBC2 |
| TCR152 | EVDPIGHLY | A0101 | TRAV12-1 | TRAJ29 | TRAC | TRBV6-1 | TRBD1 | TRBJ1-2 | TRBC1 |
| TCR153 | EVDPIGHLY | A0101 | TRAV19 | TRAJ40 | TRAC | TRBV20-1 | TRBD2 | TRBJ2-3 | TRBC2 |
| TCR154 | EVDPIGHLY | A0101 | TRAV21 | TRAJ43 | TRAC | TRBV7-3 | None | TRBJ2-2 | TRBC2 |
| TCR155 | EVDPIGHLY | A0101 | TRAV21 | TRAJ4 | TRAC | TRBV5-1 | TRBD1 | TRBJ2-1 | TRBC2 |
| TCR156 | EVDPIGHLY | A0101 | TRAV26-2 | TRAJ32 | TRAC | TRBV24-1 | TRBD1 | TRBJ2-2 | TRBC2 |
| TCR157 | EVDPIGHLY | A0101 | TRAV21 | TRAJ4 | TRAC | TRBV20-1 | TRBD2 | TRBJ1-2 | TRBC1 |
| TCR158 | EVDPIGHLY | A0101 | TRAV19 | TRAJ15 | TRAC | TRBV7-8 | TRBD1 | TRBJ2-7 | TRBC2 |
| TCR159 | EVDPIGHLY | A0101 | TRAV19 | TRAJ40 | TRAC | TRBV6-1 | TRBD2 | TRBJ2-1 | TRBC2 |
| TCR160 | EVDPIGHLY | A0101 | TRAV12-2 | TRAJ13 | TRAC | TRBV25-1 | None | TRBJ2-7 | TRBC2 |
| TCR161 | EVDPIGHLY | A0101 | TRAV29DV5 | TRAJ54 | TRAC | TRBV7-8 | None | TRBJ2-1 | TRBC2 |
| TCR162 | EVDPIGHLY | A0101 | TRAV19 | TRAJ53 | TRAC | TRBV20-1 | TRBD1 | TRBJ2-7 | TRBC2 |
| TCR163 | EVDPIGHLY | A0101 | TRAV23DV6 | TRAJ36 | TRAC | TRBV9 | TRBD2 | TRBJ1-2 | TRBC1 |
| TCR164 | EVDPIGHLY | A0101 | TRAV19 | TRAJ40 | TRAC | TRBV10-3 | None | TRBJ1-1 | TRBC1 |
| TCR165 | EVDPIGHLY | A0101 | TRAV8-6 | TRAJ32 | TRAC | TRBV19 | TRBD1 | TRBJ1-1 | TRBC1 |
| TCR166 | EVDPIGHLY | A0101 | TRAV1-1 | TRAJ13 | TRAC | TRBV14 | TRBD1 | TRBJ2-1 | TRBC2 |
| TCR167 | EVDPIGHLY | A0101 | TRAV21 | TRAJ6 | TRAC | TRBV20-1 | TRBD1 | TRBJ2-7 | TRBC2 |
| TCR168 | EVDPIGHLY | A0101 | TRAV2I | TRAJ44 | TRAC | TRBV9 | None | TRBJ2-7 | TRBC2 |
| TCR169 | EVDPIGHLY | A0101 | TRAV29DV5 | TRAJ3 | TRAC | TRBV3-1 | TRBD2 | TRBJ2-5 | TRBC2 |
| TCR170 | EVDPIGHLY | A0101 | TRAV17 | TRAJ39 | TRAC | TRBV7-2 | None | TRBJ1-2 | TRBC1 |
| TCR171 | EVDPIGHLY | A0101 | TRAV26-2 | TRAJ12 | TRAC | TRBV7-9 | TRBD1 | TRBJ1-2 | TRBC1 |
| TCR172 | EVDPIGHLY | A0101 | TRAV29DV5 | TRAJ22 | TRAC | TRBV11-3 | TRBD1 | TRBJ2-7 | TRBC2 |
| TCR173 | EVDPIGHLY | A0101 | TRAV21 | TRAJ20 | TRAC | TRBVI2-4 | TRBD2 | TRBJ2-3 | TRBC2 |
| TCR174 | EVDPIGHLY | A0101 | TRAV12-3 | TRAJ3 | TRAC | TRBV27 | TRBD1 | TRBJ2-7 | TRBC2 |
| TCR175 | EVDPIGHLY | A0101 | TRAV27 | TRAJ33 | TRAC | TRBV6-5 | TRBD2 | TRBJ2-2 | TRBC2 |
| TCR176 | EVDPIGHLY | A0101 | TRAV13-1 | TRAJ22 | TRAC | TRBV12-4 | TRBD1 | TRBJ2-3 | TRBC2 |
| TCR177 | EVDPIGHLY | A0101 | TRAV26-1 | TRAJ34 | TRAC | TRBV27 | None | TRBJ1-2 | TRBC1 |
| TCR178 | EVDPIGHLY | A0101 | TRAV10 | TRAJ4 | TRAC | TRBV7-9 | TRBD1 | TRBJ2-4 | TRBC2 |
| TCR179 | EVDPIGHLY | A0101 | TRAV21 | TRAJ6 | TRAC | TRBV20-1 | TRBD2 | TRBJ1-2 | TRBC1 |
| TCR180 | EVDPIGHLY | A0101 | TRAV12-3 | TRAJ20 | TRAC | TRBV9 | TRBD1 | TRBJ1-1 | TRBC1 |
| TCR181 | EVDPIGHLY | A0101 | TRAV21 | TRAJ26 | TRAC | TRBV10-3 | None | TRBJ1-1 | TRBC1 |
| TCR182 | EVDPIGHLY | A0101 | TRAV12-2 | TRAJ20 | TRAC | TRBVI8 | TRBD1 | TRBJ2-7 | TRBC2 |
| TCR183 | EVDPIGHLY | A0101 | TRAV9-2 | TRAJ23 | TRAC | TRBV11-3 | TRBD1 | TRBJ1-1 | TRBC1 |
| TCR184 | EVDPIGHLY | A0101 | TRAV21 | TRAJ6 | TRAC | TRBV6-1 | TRBD2 | TRBJ2-1 | TRBC2 |
| TCR185 | EVDPIGHLY | A0101 | TRAV12-3 | TRAJ20 | TRAC | TRBV7-8 | TRBD1 | TRBJ2-2 | TRBC2 |
| TCR186 | EVDPIGHLY | A0101 | TRAV9-2 | TRAJ23 | TRAC | TRBV10-3 | None | TRBJ1-1 | TRBC1 |
| TCR187 | EVDPIGHLY | A0101 | TRAV24 | TRAJ45 | TRAC | TRBV5-4 | TRBD1 | TRBJ1-4 | TRBC1 |
| TCR188 | EVDPIGHLY | A0101 | TRAV13-1 | TRAJ3 | TRAC | TRBV27 | TRBD2 | TRBJ1-1 | TRBC1 |
| TCR189 | EVDPIGHLY | A0101 | TRAV20 | TRAJ20 | TRAC | TRBV7-2 | TRBD1 | TRBJ2-7 | TRBC2 |
| TCR190 | EVDPIGHLY | A0101 | TRAV8-4 | TRAJ42 | TRAC | TRBV9 | TRBD1 | TRBJ2-1 | TRBC2 |
| TCR191 | EVDPIGHLY | A0101 | TRAV1-2 | TRAJ31 | TRAC | TRBV7-9 | TRBD1 | TRBJ1-5 | TRBC1 |
| TCR192 | EVDPIGHLY | A0101 | TRAV12-1 | TRAJ13 | TRAC | TRBV20-1 | TRBD1 | TRBJ2-7 | TRBC2 |
| TCR193 | EVDPIGHLY | A0101 | TRAV12-1 | TRAJ4 | TRAC | TRBV28 | TRBD2 | TRBJ2-7 | TRBC2 |
| TCR194 | EVDPIGHLY | A0101 | TRAV21 | TRAJ4 | TRAC | TRBV27 | TRBD2 | TRBJ2-2 | TRBC2 |
| TCR195 | EVDPIGHLY | A0101 | TRAV3 | TRAJ9 | TRAC | TRBV7-9 | TRBD1 | TRBJ2-7 | TRBC2 |
| TCR196 | EVDPIGHLY | A0101 | TRAV26-1 | TRAJ42 | TRAC | TRBVI9 | None | TRBJ2-2 | TRBC2 |
| TCR197 | EVDPIGHLY | A0101 | TRAV21 | TRAJ47 | TRAC | TRBVI9 | None | TRBJI -1 | TRBC1 |
| TCR198 | EVDPIGHLY | A0101 | TRAV26-1 | TRAJ34 | TRAC | TRBV20-1 | TRBD2 | TRBJ1-2 | TRBC1 |
| TCR199 | EVDPIGHLY | A0101 | TRAV21 | TRAJ31 | TRAC | TRBV20-1 | TRBD1 | TRBJ2-7 | TRBC2 |
| TCR200 | EVDPIGHLY | A0101 | TRAV12-1 | TRAJ11 | TRAC | TRBV20-1 | TRBD2 | TRBJ1-2 | TRBC1 |
| TCR201 | EVDPIGHLY | A0101 | TRAV17 | TRAJ34 | TRAC | TRBV6-1 | TRBD2 | TRBJ2-1 | TRBC2 |
| TCR202 | EVDPIGHLY | A0101 | TRAV13-2 | TRAJ47 | TRAC | TRBV19 | TRBD2 | TRBJ2-1 | TRBC2 |
| TCR203 | EVDPIGHLY | A0101 | TRAV29DV5 | TRAJ28 | TRAC | TRBV27 | TRBD2 | TRBJ2-4 | TRBC2 |
| TCR204 | EVDPIGHLY | A0101 | TRAV13-2 | TRAJ17 | TRAC | TRBV27 | TRBD2 | TRBJ1-5 | TRBC1 |
| TCR205 | EVDPIGHLY | A0101 | TRAV38-2DV8 | TRAJ57 | TRAC | TRBV5-4 | TRBD1 | TRBJ1-2 | TRBC1 |
| TCR206 | EVDPIGHLY | A0101 | TRAV17 | TRAJ32 | TRAC | TRBV7-8 | TRBD2 | TRBJ2-1 | TRBC2 |
| TCR207 | EVDPIGHLY | A0101 | TRAV21 | TRAJ39 | TRAC | TRBV20-1 | TRBD1 | TRBJ2-7 | TRBC2 |
| TCR208 | EVDPIGHLY | A0101 | TRAV12-3 | TRAJ20 | TRAC | TRBV7-9 | TRBD1 | TRBJ2-3 | TRBC2 |
| TCR209 | EVDPIGHLY | A0101 | TRAV1-1 | TRAJ4 | TRAC | TRBV20-1 | TRBD1 | TRBJ2-7 | TRBC2 |
| TCR210 | EVDPIGHLY | A0101 | TRAV12-1 | TRAJ9 | TRAC | TRBV2 | TRBD1 | TRBJ2-7 | TRBC2 |
| TCR211 | EVDPIGHLY | A0101 | TRAV19 | TRAJ32 | TRAC | TRBV9 | TRBD1 | TRBJ1-2 | TRBC1 |
| TCR212 | EVDPIGHLY | A0101 | TRAV8-3 | TRAJ6 | TRAC | TRBV9 | TRBD2 | TRBJ2-1 | TRBC2 |
| TCR213 | EVDPIGHLY | A0101 | TRAV19 | TRAJ40 | TRAC | TRBV7-9 | None | TRBJ2-7 | TRBC2 |
| TCR214 | EVDPIGHLY | A0101 | TRAV5 | TRAJ37 | TRAC | TRBV5-6 | TRBD2 | TRBJ1-1 | TRBC1 |
| TCR215 | EVDPIGHLY | A0101 | TRAV21 | TRAJ33 | TRAC | TRBV20-1 | TRBD2 | TRBJ1-2 | TRBC1 |
| TCR216 | EVDPIGHLY | A0101 | TRAV29DV5 | TRAJ3 | TRAC | TRBV20-1 | TRBD1 | TRBJ2-7 | TRBC2 |
| TCR217 | EVDPIGHLY | A0101 | TRAV1-1 | TRAJ4 | TRAC | TRBV20-1 | TRBD2 | TRBJ1-2 | TRBC1 |
| TCR218 | EVDPIGHLY | A0101 | TRAV 21 | TRAJ6 | TRAC | TRBV10-3 | None | TRBJ1-1 | TRBC1 |
| TCR219 | EVDPIGHLY | A0101 | TRAV19 | TRAJ23 | TRAC | TRBV9 | TRBD1 | TRBJ1-1 | TRBC1 |
| TCR220 | EVDPIGHLY | A0101 | TRAV12-2 | TRAJ20 | TRAC | TRBV11-2 | TRBD2 | TRBJ2-2 | TRBC2 |
| TCR221 | EVDPIGHLY | A0101 | TRAV1-2 | TRAJ15 | TRAC | TRBV24-1 | TRBD2 | TRBJ2-1 | TRBC2 |
| TCR222 | EVDPIGHLY | A0101 | TRAV21 | TRAJ9 | TRAC | TRBV5-4 | None | TRBJ1-6 | TRBC1 |
| TCR223 | EVDPIGHLY | A0101 | TRAV8-6 | TRAJ12 | TRAC | TRBV7-9 | TRBD1 | TRBJ2-2 | TRBC2 |
| TCR224 | EVDPIGHLY | A0101 | TRAV21 | TRAJ31 | TRAC | TRBV11-2 | TRBD2 | TRBJ1-2 | TRBC1 |
| TCR225 | EVDPIGHLY | A0101 | TRAV21 | TRAJ41 | TRAC | TRBV9 | TRBD1 | TRBJ1-1 | TRBC1 |
| TCR226 | EVDPIGHLY | A0101 | TRAV25 | TRAJ28 | TRAC | TRBV7-2 | TRBD2 | TRBJ2-6 | TRBC2 |
| TCR227 | EVDPIGHLY | A0101 | TRAV21 | TRAJ33 | TRAC | TRBV10-3 | TRBD1 | TRBJ1-3 | TRBC1 |
| TCR228 | EVDPIGHLY | A0101 | TRAV21 | TRAJ49 | TRAC | TRBV5-1 | TRBD1 | TRBJ2-5 | TRBC2 |
| TCR229 | EVDPIGHLY | A0101 | TRAV1-1 | TRAJ34 | TRAC | TRBV6-6 | None | TRBJ1-5 | TRBC1 |
| TCR230 | EVDPIGHLY | A0101 | TRAV24 | TRAJ6 | TRAC | TRBV7-2 | TRBD1 | TRBJ2-1 | TRBC2 |
| TCR231 | EVDPIGHLY | A0101 | TRAV1-1 | TRAJ15 | TRAC | TRBV6-6 | None | TRBJ1-5 | TRBC1 |
| TCR232 | EVDPIGHLY | A0101 | TRAV21 | TRAJ15 | TRAC | TRBV29-1 | None | TRBJ1-1 | TRBC1 |
| TCR233 | EVDPIGHLY | A0101 | TRAV21 | TRAJ43 | TRAC | TRBV12-4 | None | TRBJ1-5 | TRBC1 |
| TCR234 | EVDPIGHLY | A0101 | TRAV21 | TRAJ30 | TRAC | TRBV9 | TRBD1 | TRBJ1-4 | TRBC1 |
| TCR235 | EVDPIGHLY | A0101 | TRAV2I | TRAJ31 | TRAC | TRBV5-1 | TRBD1 | TRBJ2-7 | TRBC2 |
| TCR236 | EVDPIGHLY | A0101 | TRAV26-1 | TRAJ45 | TRAC | TRBV19 | TRBD2 | TRBJ2-1 | TRBC2 |
| TCR237 | EVDPIGHLY | A0101 | TRAV21 | TRAJ43 | TRAC | TRBV24-1 | TRBD2 | TRBJ2-1 | TRBC2 |
| TCR238 | EVDPIGHLY | A0101 | TRAV21 | TRAJ31 | TRAC | TRBV24-1 | TRBD2 | TRBJ2-1 | TRBC2 |
| TCR239 | EVDPIGHLY | A0101 | TRAV29DV5 | TRAJ28 | TRAC | TRBV4-1 | TRBD1 | TRBJ1-4 | TRBC1 |
| TCR240 | EVDPIGHLY | A0101 | TRAV26-2 | TRAJ44 | TRAC | TRBV27 | None | TRBJ2-1 | TRBC2 |
| TCR241 | EVDPIGHLY | A0101 | TRAV21 | TRAJ31 | TRAC | TRBV9 | TRBD1 | TRBJ1-5 | TRBC1 |
| TCR242 | EVDPIGHLY | A0101 | TRAV21 | TRAJ36 | TRAC | TRBV9 | TRBD1 | TRBJ1-2 | TRBC1 |
| TCR243 | EVDPIGHLY | A0101 | TRAV21 | TRAJ9 | TRAC | TRBV9 | TRBD1 | TRBJ1-1 | TRBC1 |
| TCR244 | EVDPIGHLY | A0101 | TRAV8-3 | TRAJ15 | TRAC | TRBV4-1 | None | TRBJ2-1 | TRBC2 |
| TCR245 | EVDPIGHLY | A0101 | TRAV21 | TRAJ43 | TRAC | TRBV24-1 | TRBD1 | TRBJ2-3 | TRBC2 |
| TCR246 | EVDPIGHLY | A0101 | TRAV29DV5 | TRAJ40 | TRAC | TRBV7-9 | TRBD1 | TRBJ1-6 | TRBC1 |
| TCR247 | EVDPIGHLY | A0101 | TRAV30 | TRAJ32 | TRAC | TRBV28 | TRBD1 | TRBJ1-1 | TRBC1 |
| TCR248 | EVDPIGHLY | A0101 | TRAV38-2DV8 | TRAJ26 | TRAC | TRBV7-9 | TRBD2 | TRBJ2-5 | TRBC2 |
| TCR249 | EVDPIGHLY | A0101 | TRAV12-1 | TRAJ6 | TRAC | TRBV20-1 | TRBD1 | TRBJ1-3 | TRBC1 |
| TCR250 | EVDPIGHLY | A0101 | TRAV21 | TRAJ47 | TRAC | TRBV5-1 | None | TRBJ1-1 | TRBC1 |
| TCR251 | EVDPIGHLY | A0101 | TRAV38-2DV8 | TRAJ45 | TRAC | TRBV29-1 | TRBD1 | TRBJ2-7 | TRBC2 |
| TCR252 | EVDPIGHLY | A0101 | TRAV21 | TRAJ15 | TRAC | TRBV7-2 | None | TRBJ1-1 | TRBC1 |
| TCR253 | EVDPIGHLY | A0101 | TRAV12-2 | TRAJ29 | TRAC | TRBV9 | TRBD1 | TRBJ1-1 | TRBC1 |
| TCR254 | EVDPIGHLY | A0101 | TRAV3 | TRAJ6 | TRAC | TRBV28 | TRBD1 | TRBJ2-7 | TRBC2 |
| TCR255 | EVDPIGHLY | A0101 | TRAV21 | TRAJ9 | TRAC | TRBV10-3 | TRBD1 | TRBJ1-3 | TRBC1 |
| TCR256 | EVDPIGHLY | A0101 | TRAV1-2 | TRAJ15 | TRAC | TRBV7-9 | TRBD1 | TRBJ2-2 | TRBC2 |
| T1R257 | EVDPIGHLY | A0101 | TRAV8-6 | TRAJ40 | TRAC | TRBV15 | None | TRBJ2-5 | TRBC2 |
| TCR258 | EVDPIGHLY | A0101 | TRAV38-2DV8 | TRAJ57 | TRAC | TRBV13 | TRBD1 | TRBJ1-4 | TRBC1 |
| TCR259 | EVDPIGHLY | A0101 | TRAV8-6 | TRAJ10 | TRAC | TRBV7-9 | None | TRBJ1-1 | TRBC1 |
| TCR260 | EVDPIGHLY | A0101 | TRAV21 | TRAJ20 | TRAC | TRBV5-4 | TRBD1 | TRBJ1-5 | TRBC1 |
| TCR261 | EVDPIGHLY | A0101 | TRAV13-1 | TRAJ28 | TRAC | TRBV7-8 | TRBD1 | TRBJ1-5 | TRBC1 |
| TCR262 | EVDPIGHLY | A0101 | TRAV21 | TRAJ9 | TRAC | TRBV24-1 | TRBD2 | TRBJ2-1 | TRBC2 |
| TCR263 | EVDPIGHLY | A0101 | TRAV1-2 | TRAJ15 | TRAC | TRBV2 | TRBD2 | TRBJ2-1 | TRBC2 |
| TCR264 | EVDPIGHLY | A0101 | TRAV35 | TRAJ26 | TRAC | TRBV27 | TRBD1 | TRBJ1-1 | TRBC1 |
| TCR265 | EVDPIGHLY | A0101 | TRAV38-2DV8 | TRAJ43 | TRAC | TRBV5-1 | TRBD2 | TRBJ2-5 | TRBC2 |
| TCR266 | EVDPIGHLY | A0101 | TRAV5 | TRAJ32 | TRAC | TRBV19 | TRBD2 | TRBJ2-7 | TRBC2 |
| TCR267 | EVDPIGHLY | A0101 | TRAV13-1 | TRAJ21 | TRAC | TRBV5-1 | TRBD2 | TRBJ2-7 | TRBC2 |
| TCR268 | EVDPIGHLY | A0101 | TRAV12-2 | TRAJ45 | TRAC | TRBVI2-4 | TRBD1 | TRBJ2-1 | TRBC2 |
| TCR269 | EVDPIGHLY | A0101 | TRAV21 | TRAJ31 | TRAC | TRBVI2-5 | None | TRBJ2-2 | TRBC2 |
| TCR270 | EVDPIGHLY | A0101 | TRAV24 | TRAJ52 | TRAC | TRBV27 | None | TRBJ2-1 | TRBC2 |
| TCR271 | EVDPIGHLY | A0101 | TRAV21 | TRAJ52 | TRAC | TRBV19 | TRBD1 | TRBJ1-1 | TRBC1 |
| TCR272 | EVDPIGHLY | A0101 | TRAV36DV7 | TRAJ44 | TRAC | TRBV7-9 | TRBD1 | TRBJ2-2 | TRBC2 |
| TCR273 | EVDPIGHLY | A0101 | TRAV3 | TRAJ29 | TRAC | TRBV11-2 | TRBD1 | TRBJ2-5 | TRBC2 |
| TCR274 | EVDPIGHLY | A0101 | TRAV1-1 | TRAJ15 | TRAC | TRBVI3 | TRBD1 | TRBJ1-2 | TRBC1 |
| TCR275 | EVDPIGHLY | A0101 | TRAV29DV5 | TRAJ52 | TRAC | TRBV11-3 | TRBD1 | TRBJ2-3 | TRBC2 |
| TCR276 | EVDPIGHLY | A0101 | TRAV12-1 | TRAJ6 | TRAC | TRBVI9 | TRBD1 | TRBJ1-1 | TRBC1 |
| TCR277 | EVDPIGHLY | A0101 | TRAV19 | TRAJ13 | TRAC | TRBV27 | TRBD2 | TRBJ2-7 | TRBC2 |
| TCR278 | EVDPIGHLY | A0101 | TRAV17 | TRAJ43 | TRAC | TRBV12-3 | None | TRBJ1-4 | TRBC1 |
| TCR279 | EVDPIGHLY | A0101 | TRAV12-3 | TRAJ20 | TRAC | TRBV12-4 | None | TRBJ2-1 | TRBC2 |
| TCR280 | EVDPIGHLY | A0101 | TRAV21 | TRAJ52 | TRAC | TRBV4-1 | TRBD2 | TRBJ2-7 | TRBC2 |
| TCR281 | EVDPIGHLY | A0101 | TRAV21 | TRAJ23 | TRAC | TRBV19 | TRBD1 | TRBJ2-7 | TRBC2 |
| TCR282 | EVDPIGHLY | A0101 | TRAV1-1 | TRAJ30 | TRAC | TRBV13 | TRBD1 | TRBJ1-2 | TRBC1 |
| TCR283 | EVDPIGHLY | A0101 | TRAV12-2 | TRAJ43 | TRAC | TRBV12-4 | TRBD2 | TRBJ2-7 | TRBC2 |
| TCR284 | EVDPIGHLY | A0101 | TRAV24 | TRAJ10 | TRAC | TRBV5-1 | TRBD1 | TRBJ1-2 | TRBC1 |
| TCR285 | EVDPIGHLY | A0101 | TRAV5 | TRAJ9 | TRAC | TRBV4-1 | TRBD1 | TRBJ1-1 | TRBC1 |
| TCR286 | EVDPIGHLY | A0101 | TRAV21 | TRAJ40 | TRAC | TRBV7-8 | None | TRBJ1-1 | TRBC1 |
| TCR287 | EVDPIGHLY | A0101 | TRAV13-1 | TRAJ45 | TRAC | TRBV9 | TRBD1 | TRBJ1-6 | TRBC1 |
| TCR288 | EVDPIGHLY | A0101 | TRAV12-1 | TRAJ26 | TRAC | TRBV4-1 | TRBD1 | TRBJ2-7 | TRBC2 |
| TCR289 | EVDPIGHLY | A0101 | TRAV26-2 | TRAJ45 | TRAC | TRBV19 | None | TRBJ1-2 | TRBC1 |
| TCR290 | EVDPIGHLY | A0101 | TRAV22 | TRAJ23 | TRAC | TRBV5-4 | TRBD1 | TRBJ1-1 | TRBC1 |
| TCR291 | EVDPIGHLY | A0101 | TRAV19 | TRAJ42 | TRAC | TRBV28 | None | TRBJ2-7 | TRBC2 |
| TCR292 | EVDPIGHLY | A0101 | TRAV17 | TRAJ52 | TRAC | TRBV7-8 | TRBD1 | TRBJ1-2 | TRBC1 |
| TCR293 | EVDPIGHLY | A0101 | TRAV12-1 | TRAJ39 | TRAC | TRBV3-1 | TRBD1 | TRBJ2-3 | TRBC2 |
| TCR294 | EVDPIGHLY | A0101 | TRAV21 | TRAJ9 | TRAC | TRBV5-1 | TRBD1 | TRBJ2-7 | TRBC2 |
| TCR295 | EVDPIGHLY | A0101 | TRAV1-1 | TRAJ5 | TRAC | TRBV24-1 | TRBD2 | TRBJ2-1 | TRBC2 |
| TCR296 | EVDPIGHLY | A0101 | TRAV23DV6 | TRAJ13 | TRAC | TRBV6-5 | TRBD1 | TRBJ2-1 | TRBC2 |
| TCR297 | EVDPIGHLY | A0101 | TRAV8-6 | TRAJ12 | TRAC | TRBV24-1 | TRBD2 | TRBJ2-1 | TRBC2 |
| TCR298 | EVDPIGHLY | A0101 | TRAV1-2 | TRAJ28 | TRAC | TRBV27 | None | TRBJ2-3 | TRBC2 |
| TCR299 | EVDPIGHLY | A0101 | TRAV29DV5 | TRAJ34 | TRAC | TRBV4-1 | TRBD2 | TRBJ2-3 | TRBC2 |
| TCR300 | EVDPIGHLY | A0101 | TRAV12-1 | TRAJ21 | TRAC | TRBV28 | TRBD1 | TRBJ1-5 | TRBC1 |
| TCR301 | EVDPIGHLY | A0101 | TRAV9-2 | TRAJ29 | TRAC | TRBV5-8 | TRBD2 | TRBJ1-1 | TRBC1 |
| TCR302 | EVDPIGHLY | A0101 | TRAV27 | TRAJ40 | TRAC | TRBV7-6 | None | TRBJ2-4 | TRBC2 |
| TCR303 | EVDPIGHLY | A0101 | TRAV21 | TRAJ31 | TRAC | TRBV7-8 | TRBD1 | TRBJ1-2 | TRBC1 |
| TCR304 | EVDPIGHLY | A0101 | TRAV21 | TRAJ30 | TRAC | TRBV9 | TRBD1 | TRBJ1-2 | TRBC1 |
| TCR305 | EVDPIGHLY | A0101 | TRAV19 | TRAJ30 | TRAC | TRBV20-1 | None | TRBJ2-1 | TRBC1 |
| TCR306 | EVDPIGHLY | A0101 | TRAV1-1 | TRAJ26 | TRAC | TRBV12-5 | TRBD2 | TRBJ2-1 | TRBC2 |
| TCR307 | EVDPIGHLY | A0101 | TRAV1-2 | TRAJ33 | TRAC | TRBV9 | TRBD2 | TRBJ2-3 | TRBC2 |
| TCR308 | EVDPIGHLY | A0101 | TRAV26-1 | TRAJ50 | TRAC | TRBV27 | TRBD1 | TRBJ2-3 | TRBC2 |
| TCR309 | EVDPIGHLY | A0101 | TRAV40 | TRAJ41 | TRAC | TRBV6-5 | TRBD2 | TRBJ1-2 | TRBC1 |
| TCR310 | EVDPIGHLY | A0101 | TRAV12-2 | TRAJ31 | TRAC | TRBV7-9 | TRBD1 | TRBJ1-5 | TRBC1 |
| TCR311 | EVDPIGHLY | A0101 | TRAV5 | TRAJ43 | TRAC | TRBV5-1 | TRBD1 | TRBJ2-3 | TRBC2 |
| TCR312 | EVDPIGHLY | A0101 | TRAV24 | TRAJ52 | TRAC | TRBV5-1 | None | TRBJ1-1 | TRBC1 |
| TCR313 | EVDPIGHLY | A0101 | TRAV1-2 | TRAJ11 | TRAC | TRBV7-6 | TRBD1 | TRBJ1-3 | TRBC1 |
| TCR314 | EVDPIGHLY | A0101 | TRAV21 | TRAJ33 | TRAC | TRBV5-1 | TRBD1 | TRBJ2-7 | TRBC2 |
| TCR315 | EVDPIGHLY | A0101 | TRAV21 | TRAJ39 | TRAC | TRBV10-3 | None | TRBJ2-1 | TRBC2 |
| TCR316 | EVDPIGHLY | A0101 | TRAV21 | TRAJ20 | TRAC | TRBV14 | TRBD1 | TRBJ1-2 | TRBC1 |
| TCR317 | EVDPIGHLY | A0101 | TRAV29DV5 | TRAJ48 | TRAC | TRBV7-9 | TRBD1 | TRBJ1-2 | TRBC1 |
| TCR318 | EVDPIGHLY | A0101 | TRAV13-1 | TRAJ22 | TRAC | TRBV291- | None | TRBJ1-1 | TRBC1 |
| TCR319 | EVDPIGHLY | A0101 | TRAV21 | TRAJ33 | TRAC | TRBV10-3 | None | TRBJ2-1 | TRBC2 |
| TCR320 | EVDPIGHLY | A0101 | TRAV39 | TRAJ49 | TRAC | TRBV24-1 | TRBD1 | TRBJ1-4 | TRBC1 |
| TCR321 | EVDPIGHLY | A0101 | TRAV13-1 | TRAJ23 | TRAC | TRBV27 | TRBD1 | TRBJ1-2 | TRBC1 |
| TCR322 | EVDPIGHLY | A0101 | TRAV21 | TRAJ9 | TRAC | TRBV9 | TRBD1 | TRBJ2-7 | TRBC2 |
| TCR323 | EVDPIGHLY | A0101 | TRAV21 | TRAJ33 | TRAC | TRBV9 | None | TRBJ1-1 | TRBC1 |
| TCR324 | EVDPIGHLY | A0101 | TRAV19 | TRAJ28 | TRAC | TRBV19 | TRBD1 | TRBJ1-4 | TRBC1 |
| TCR325 | EVDPIGHLY | A0101 | TRAV10 | TRAJ8 | TRAC | TRBV5-1 | TRBD1 | TRBJ2-7 | TRBC2 |
| TCR326 | EVDPIGHLY | A0101 | TRAV21 | TRAJ48 | TRAC | TRBV27 | TRBD2 | TRBJ2-2 | TRBC2 |
| TCR327 | EVDPIGHLY | A0101 | TRAV12-2 | TRAJ4 | TRAC | TRBV7-2 | TRBD2 | TRBJ2-1 | TRBC2 |
| TCR328 | EVDPIGHLY | A0101 | TRAV21 | TRAJ31 | TRAC | TRBV5-1 | None | TRBJ1-1 | TRBC1 |
| TCR329 | EVDPIGHLY | A0101 | TRAV21 | TRAJ33 | TRAC | TRBV9 | None | TRBJ1-1 | TRBC1 |
| TCR330 | EVDPIGHLY | A0101 | TRAV21 | TRAJ6 | TRAC | TRBV6-6 | TRBD1 | TRBJ1-5 | TRBC1 |
| TCR331 | EVDPIGHLY | A0101 | TRAV21 | TRAJ29 | TRAC | TRBV5-1 | TRBD2 | TRBJ2-5 | TRBC2 |
| TCR332 | EVDPIGHLY | A0101 | TRAV41 | TRAJ41 | TRAC | TRBV7-9 | TRBD1 | TRBJ1-2 | None |
| TCR333 | EVDPIGHLY | A0101 | TRAV21 | TRAJ33 | None | TRBV5-1 | None | TRBJ1-1 | TRBC1 |
| TCR334 | EVDPIGHLY | A0101 | TRAV17 | TRAJ39 | TRAC | TRBV27 | TRBD2 | TRBJ2-1 | TRBC2 |
| TCR335 | EVDPIGHLY | A0101 | TRAV13-2 | TRAJ13 | TRAC | TRBV9 | None | TRBJ1-3 | TRBC1 |
| TCR336 | EVDPIGHLY | A0101 | TRAV21 | TRAJ33 | TRAC | TRBV5-1 | TRBD2 | TRBJ2-5 | TRBC2 |
| TCR337 | EVDPIGHLY | A0101 | TRAV17 | TRAJ57 | TRAC | TRBV9 | TRBD2 | TRBJ1-1 | TRBC1 |
| TCR338 | EVDPIGHLY | A0101 | TRAV5 | TRAJ44 | TRAC | TRBV7-9 | TRBD1 | TRBJ1-1 | TRBC1 |
| TCR339 | EVDPIGHLY | A0101 | TRAV3 | TRAJ39 | TRAC | TRBV27 | TRBD1 | TRBJ1-5 | TRBC1 |
| TCR340 | EVDPIGHLY | A0101 | TRAV1-2 | TRAJ4 | TRAC | TRBV11-1 | TRBD2 | TRBJ2-1 | TRBC2 |
| TCR341 | EVDPIGHLY | A0101 | TRAV38-2DV8 | TRAJ40 | TRAC | TRBV7-8 | TRBD1 | TRBJ2-4 | TRBC2 |
| TCR342 | EVDPIGHLY | A0101 | TRAV8-3 | TRAJ41 | TRAC | TRBV7-9 | None | TRBJ1-1 | TRBC1 |
| TCR343 | EVDPIGHLY | A0101 | TRAV5 | TRAJ4 | TRAC | TRBV11-2 | None | TRBJ2-1 | TRBC2 |
| TCR344 | EVDPIGHLY | A0101 | TRAV24 | TRAJ49 | TRAC | TRBV6-5 | TRBD1 | TRBJ1-1 | TRBC1 |
| TCR345 | EVDPIGHLY | A0101 | TRAV4 | TRAJ45 | TRAC | TRBV24-1 | TRBD2 | TRBJ1-1 | TRBC1 |
| TCR346 | EVDPIGHLY | A0101 | TRAV29DV5 | TRAJ48 | TRAC | TRBV20-1 | TRBD2 | TRBJ2-7 | TRBC2 |
| TCR347 | EVDPIGHLY | A0101 | TRAV26-2 | TRAJ44 | TRAC | TRBV6-1 | None | TRBJ2-7 | TRBC2 |
| TCR348 | EVDPIGHLY | A0101 | TRAV21 | TRAJ27 | TRAC | TRBV7-9 | None | TRBJ1-6 | TRBC1 |
| TCR349 | EVDPIGHLY | A0101 | TRAV26-1 | TRAJ49 | TRAC | TRBV7-9 | None | TRBJ2-7 | TRBC2 |
| TCR350 | EVDPIGHLY | A0101 | TRAV12-1 | TRAJ5 | TRAC | TRBV7-8 | TRBD1 | TRBJ2-1 | TRBC2 |
| TCR351 | EVDPIGHLY | A0101 | TRAV21 | TRAJ33 | TRAC | TRBV9 | TRBD1 | TRBJ2-7 | TRBC2 |
| TCR352 | EVDPIGHLY | A0101 | TRAV21 | TRAJ20 | TRAC | TRBV27 | TRBD1 | TRBJ2-4 | TRBC2 |
| TCR353 | EVDPIGHLY | A0101 | TRAV39 | TRAJ42 | TRAC | TRBV9 | TRBD1 | TRBJ2-7 | TRBC2 |
| TCR354 | EVDPIGHLY | A0101 | TRAV1-2 | TRAJ39 | TRAC | TRBV27 | TRBD2 | TRBJ1-4 | TRBC1 |
| TCR355 | EVDPIGHLY | A0101 | TRAV1-1 | TRAJ34 | TRAC | TRBV9 | TRBD1 | TRBJ2-3 | TRBC2 |
| TCR356 | EVDPIGHLY | A0101 | TRAV25 | TRAJ34 | TRAC | TRBV29-1 | TRBD1 | TRBJ1-2 | TRBC1 |
| TCR357 | EVDPIGHLY | A0101 | TRAV39 | TRAJ39 | TRAC | TRBV30 | TRBD1 | TRBJ2-1 | TRBC2 |
| TCR358 | EVDPIGHLY | A0101 | TRAV21 | TRAJ6 | TRAC | TRBV20-1 | TRBD2 | TRBJ1-1 | TRBC1 |
| TCR359 | EVDPIGHLY | A0101 | TRAV8-6 | TRAJ30 | TRAC | TRBV9 | TRBD2 | TRBJ2-2 | TRBC2 |
| TCR360 | EVDPIGHLY | A0101 | TRAV21 | TRAJ18 | TRAC | TRBV27 | TRBD1 | TRBJ1-2 | TRBC1 |
| TCR361 | EVDPIGHLY | A0101 | TRAV12-3 | TRAJ23 | TRAC | TRBV11-3 | TRBD1 | TRBJ2-2 | TRBC2 |
| TCR362 | EVDPIGHLY | A0101 | TRAV12-1 | TRAJ47 | TRAC | TRBV5-6 | None | TRBJ1-2 | TRBC1 |
| TCR363 | EVDPIGHLY | A0101 | TRAV22 | TRAJ31 | TRAC | TRBV5-6 | None | TRBJ2-7 | TRBC2 |
| TCR364 | EVDPIGHLY | A0101 | TRAV21 | TRAJ33 | TRAC | TRBV14 | TRBD1 | TRBJ1-2 | TRBC1 |
| TCR365 | EVDPIGHLY | A0101 | TRAV1-2 | TRAJ31 | TRAC | TRBV2 | TRBD2 | TRBJ2-7 | TRBC2 |
| TCR366 | EVDPIGHLY | A0101 | TRAV1-2 | TRAJ5 | TRAC | TRBV20-1 | TRBD2 | TRBJ2-7 | TRBC2 |
| TCR367 | EVDPIGHLY | A0101 | TRAV21 | TRAJ33 | TRAC | TRBV5-1 | TRBD1 | TRBJ1-2 | TRBC1 |
| TCR368 | EVDPIGHLY | A0101 | TRAV16 | TRAJ28 | TRAC | TRBV7-9 | TRBD1 | TRBJ2-1 | TRBC2 |
| TCR369 | EVDPIGHLY | A0101 | TRAV13-1 | TRAJ12 | TRAC | TRBV20-1 | TRBD2 | TRBJ1-1 | TRBC1 |
| TCR370 | EVDPIGHLY | A0101 | TRAV17 | TRAJ52 | TRAC | TRBV29-1 | TRBD2 | TRBJ2-1 | TRBC2 |
| TCR371 | EVDPIGHLY | A0101 | TRAV36DV7 | TRAJ49 | TRAC | TRBV15 | TRBD2 | TRBJ2-3 | TRBC2 |
| TCR372 | EVDPIGHLY | A0101 | TRAV12-3 | TRAJ58 | TRAC | TRBV12-4 | TRBD2 | TRBJ2-1 | TRBC2 |
| TCR373 | EVDPIGHLY | A0101 | TRAV16 | TRAJ18 | TRAC | TRBV27 | TRBD1 | TRBJ2-7 | TRBC2 |
| TCR374 | EVDPIGHLY | A0101 | TRAV21 | TRAJ33 | TRAC | TRBV27 | TRBD2 | TRBJ2-2 | TRBC2 |
| TCR375 | EVDPIGHLY | A0101 | TRAV12-2 | TRAJ48 | TRAC | TRBV27 | None | TRBJ2-6 | TRBC2 |
| TCR376 | EVDPIGHLY | A0101 | TRAV21 | TRAJ33 | TRAC | TRBV2 | TRBD1 | TRBJ1-2 | TRBC1 |
| TCR377 | EVDPIGHLY | A0101 | TRAV29DV5 | TRAJ37 | TRAC | TRBV5-4 | TRBD2 | TRBJ2-7 | TRBC2 |
| TCR378 | EVDPIGHLY | A0101 | TRAV21 | TRAJ20 | TRAC | TRBV24-1 | TRBD1 | TRBJ1-4 | TRBC1 |
| TCR379 | EVDPIGHLY | A0101 | TRAV12-2 | TRAJ6 | TRAC | TRBV15 | TRBD1 | TRBJ2-2 | TRBC2 |
| TCR380 | EVDPIGHLY | A0101 | TRAV12-1 | TRAJ42 | TRAC | TRBV27 | TRBD1 | TRBJ1-5 | TRBC1 |
| TCR381 | EVDPIGHLY | A0101 | TRAV1-1 | TRAJ23 | TRAC | TRBV25-1 | TRBD1 | TRBJ2-7 | TRBC2 |
| TCR382 | EVDPIGHLY | A0101 | TRAV38-1 | TRAJ28 | TRAC | TRBV5-1 | TRBD1 | TRBJ2-1 | TRBC2 |
| TCR383 | EVDPIGHLY | A0101 | TRAV21 | TRAJ33 | TRAC | TRBV2 | TRBD1 | TRBJ1-1 | TRBC1 |
| TCR384 | EVDPIGHLY | A0101 | TRAV21 | TRAJ31 | TRAC | TRBV5-1 | TRBD1 | TRBJ2-7 | TRBC2 |
| TCR385 | EVDPIGHLY | A0101 | TRAV8-6 | TRAJ42 | TRAC | TRBV27 | None | TRBJ1-1 | TRBC1 |
| TCR386 | EVDPIGHLY | A0101 | TRAV40 | TRAJ32 | TRAC | TRBV7-6 | None | TRBJ2-2 | TRBC2 |
| TCR387 | EVDPIGHLY | A0101 | TRAV5 | TRAJ5 | TRAC | TRBV20-1 | TRBD1 | TRBJ2-5 | TRBC2 |
| TCR388 | EVDPIGHLY | A0101 | TRAV12-1 | TRAJ40 | TRAC | TRBV4-1 | None | TRBJ2-5 | TRBC2 |
| TCR389 | EVDPIGHLY | A0101 | TRAV13-2 | TRAJ53 | TRAC | TRBV5-1 | None | TRBJ1-1 | TRBC1 |
| TCR390 | EVDPIGHLY | A0101 | TRAV12-2 | TRAJ48 | TRAC | TRBV5-6 | TRBD1 | TRBJ2-2 | TRBC2 |
| TCR391 | EVDPIGHLY | A0101 | TRAV12-3 | TRAJ15 | TRAC | TRBV20-1 | None | TRBJ2-7 | TRBC2 |
| TCR392 | EVDPIGHLY | A0101 | TRAV12-3 | TRAJ23 | TRAC | TRBV13 | TRBD1 | TRBJ2-3 | TRBC2 |
| TCR393 | EVDPIGHLY | A0101 | TRAV13-2 | TRAJ9 | TRAC | TRBV7-3 | None | TRBJ1-6 | TRBC1 |
| TCR394 | EVDPIGHLY | A0101 | TRAV21 | TRAJ45 | TRAC | TRBV5-1 | None | TRBJ1-1 | TRBC1 |
| TCR395 | EVDPIGHLY | A0101 | TRAV25 | TRAJ31 | TRAC | TRBV29-1 | TRBD1 | TRBJ1-2 | TRBC1 |
| TCR396 | EVDPIGHLY | A0101 | TRAV34 | TRAJ37 | TRAC | TRBV28 | None | TRBJ1-1 | TRBC1 |
| TCR397 | EVDPIGHLY | A0101 | TRAV1-2 | TRAJ9 | TRAC | TRBV9 | TRBD1 | TRBJ2-6 | TRBC2 |
| TCR398 | EVDPIGHLY | A0101 | TRAV21 | TRAJ36 | TRAC | TRBV9 | TRBD1 | TRBJ2-7 | TRBC2 |
| TCR399 | EVDPIGHLY | A0101 | TRAV12-1 | TRAJ34 | TRAC | TRBV6-1 | None | TRBJ2-7 | TRBC2 |
| TCR400 | EVDPIGHLY | A0101 | TRAV12-1 | TRAJ26 | TRAC | TRBV11-3 | TRBD1 | TRBJ1-1 | TRBC1 |
| TCR401 | EVDPIGHLY | A0101 | TRAV17 | TRAJ36 | TRAC | TRBV5-4 | None | TRBJ2-1 | TRBC2 |
| TCR402 | EVDPIGHLY | A0101 | TRAV21 | TRAJ49 | TRAC | TRBV4-1 | TRBD1 | TRBJ1-1 | TRBC1 |
| TCR403 | EVDPIGHLY | A0101 | TRAV12-1 | TRAJ13 | TRAC | TRBV9 | TRBD2 | TRBJ2-7 | TRBC2 |
| TCR404 | EVDPIGHLY | A0101 | TRAV24 | TRAJ7 | TRAC | TRBV7-9 | TRBD1 | TRBJ2-1 | TRBC2 |
| TCR405 | EVDPIGHLY | A0101 | TRAV21 | TRAJ20 | TRAC | TRBV9 | TRBD2 | TRBJ1-1 | TRBC1 |
| TCR406 | EVDPIGHLY | A0101 | TRAV13-2 | TRAJ49 | TRAC | TRBV6-1 | TRBD1 | TRBJ2-5 | TRBC2 |
| TCR407 | EVDPIGHLY | A0101 | TRAV21 | TRAJ33 | TRAC | TRBV5-5 | TRBD1 | TRBJI-2 | TRBC1 |
| TCR408 | EVDPIGHLY | A0101 | TRAV12-1 | TRAJ39 | TRAC | TRBV4-2 | TRBD2 | TRBJ2-7 | TRBC2 |
| TCR409 | EVDPIGHLY | A0101 | TRAV26-2 | TRAJ30 | TRAC | TRBV9 | TRBD1 | TRBJ2-7 | TRBC2 |
| TCR410 | EVDPIGHLY | A0101 | TRAV20 | TRAJ45 | TRAC | TRBV5-4 | TRBD1 | TRBJ2-7 | TRBC2 |
| TCR411 | EVDPIGHLY | A0101 | TRAV21 | TRAJ31 | TRAC | TRBV7-8 | TRBD2 | TRBJ1-2 | TRBC1 |
| TCR412 | EVDPIGHLY | A0101 | TRAV38-2DV8 | TRAJ48 | TRAC | TRBV2 | TRBD1 | TRBJ1-5 | TRBC1 |
| TCR413 | EVDPIGHLY | A0101 | TRAV25 | TRAJ15 | TRAC | TRBV9 | TRBD1 | TRBJ2-1 | TRBC2 |
| TCR414 | EVDPIGHLY | A0101 | TRAV21 | TRAJ49 | TRAC | TRBV5-4 | None | TRBJ2-7 | TRBC2 |
| TCR415 | EVDPIGHLY | A0101 | TRAV21 | TRAJ12 | TRAC | TRBV27 | TRBD1 | TRBJ2-2 | TRBC2 |
| TCR416 | EVDPIGHLY | A0101 | TRAV38-2DV8 | TRAJ54 | TRAC | TRBV24-1 | None | TRBJ2-2 | TRBC2 |
| TCR417 | EVDPIGHLY | A0101 | TRAV17 | TRAJ52 | TRAC | TRBV27 | None | TRBJ2-1 | TRBC2 |
| TCR418 | EVDPIGHLY | A0101 | TRAV21 | TRAJ28 | TRAC | TRBV9 | TRBD2 | TRBJ2-7 | TRBC2 |
| TCR419 | EVDPIGHLY | A0101 | TRAV21 | TRAJ36 | TRAC | TRBV4-1 | TRBD1 | TRBJ1-1 | TRBC1 |
| TCR420 | EVDPIGHLY | A0101 | TRAV21 | TRAJ31 | TRAC | TRBV5-4 | None | TRBJ1-2 | TRBC1 |
| TCR421 | EVDPIGHLY | A0101 | TRAV21 | TRAJ33 | TRAC | TRBV5-1 | TRBD1 | TRBJ2-3 | TRBC2 |
| TCR422 | EVDPIGHLY | A0101 | TRAV12-1 | TRAJ43 | TRAC | TRBV6-5 | TRBD1 | TRBJ2-7 | TRBC2 |
| TCR423 | EVDPIGHLY | A0101 | TRAV21 | TRAJ41 | TRAC | TRBV9 | None | TRBJ2-2 | TRBC2 |
| TCR424 | EVDPIGHLY | A0101 | TRAV19 | TRAJ40 | TRAC | TRBV20-1 | None | TRBJ2-7 | TRBC2 |
| TCR425 | EVDPIGHLY | A0101 | TRAV12-2 | TRAJ52 | TRAC | TRBV6-1 | TRBD2 | TRBJ2-7 | TRBC1 |
| TCR426 | EVDPIGHLY | A0101 | TRAV26-1 | TRAJ57 | TRAC | TRBV2 | None | TRBJ2-7 | TRBC2 |
| TCR427 | EVDPIGHLY | A0101 | TRAV21 | TRAJ36 | TRAC | TRBV12-4 | TRBD1 | TRBJ1-6 | TRBC1 |
| TCR428 | EVDPIGHLY | A0101 | TRAV8-4 | TRAJ34 | TRAC | TRBV7-9 | None | TRBJ2-7 | TRBC2 |
| TCR429 | EVDPIGHLY | A0101 | TRAV19 | TRAJ32 | TRAC | TRBV7-9 | None | TRBJ1-2 | TRBC2 |
| TCR430 | EVDPIGHLY | A0101 | TRAV21 | TRAJ6 | TRAC | TRBV3-1 | TRBD2 | TRBJ1-4 | TRBC1 |
| TCR431 | EVDPIGHLY | A0101 | TRAV13-2 | TRAJ29 | TRAC | TRBV5-1 | None | TRBJ2-2 | TRBC2 |
| TCR432 | EVDPIGHLY | A0101 | TRAV14DV4 | TRAJ26 | TRAC | TRBV7-9 | TRBD1 | TRBJ2-5 | TRBC2 |
| TCR433 | EVDPIGHLY | A0101 | TRAV35 | TRAJ44 | TRAC | TRBV27 | TRBD1 | TRBJ2-1 | TRBC2 |
| TCR434 | EVDPIGHLY | A0101 | TRAV21 | TRAJ24 | TRAC | TRBV27 | TRBD1 | TRBJ1-6 | TRBC1 |
| TCR435 | EVDPIGHLY | A0101 | TRAV25 | TRAJ21 | TRAC | TRBV28 | TRBD1 | TRBJ2-7 | TRBC2 |
| TCR436 | EVDPIGHLY | A0101 | TRAV3 | TRAJ36 | TRAC | TRBV28 | None | TRBJ1-5 | TRBC1 |
| TCR437 | EVDPIGHLY | A0101 | TRAV26-2 | TRAJ52 | TRAC | TRBV5-6 | TRBD2 | TRBJ2-1 | TRBC2 |
| TCR438 | EVDPIGHLY | A0101 | TRAV8-6 | TRAJ40 | TRAC | TRBV9 | TRBD1 | TRBJ2-7 | TRBC2 |
| TCR439 | EVDPIGHLY | A0101 | TRAV21 | TRAJ42 | TRAC | TRBV28 | TRBD1 | TRBJ2-7 | TRBC2 |
| TCR440 | EVDPIGHLY | A0101 | TRAV12-1 | TRAJ32 | TRAC | TRBV20-1 | TRBD1 | TRBJ1-1 | TRBC1 |
| TCR441 | EVDPIGHLY | A0101 | TRAV24 | TRAJ24 | TRAC | TRBV28 | TRBD2 | TRBJ2-5 | TRBC2 |
| TCR442 | EVDPIGHLY | A0101 | TRAV21 | TRAJ36 | TRAC | TRBV9 | TRBD2 | TRBJ1-1 | TRBC1 |
| TCR443 | EVDPIGHLY | A0101 | TRAV12-1 | TRAJ26 | TRAC | TRBV2 | None | TRBJ1-6 | TRBC1 |
| TCR444 | EVDPIGHLY | A0101 | TRAV21 | TRAJ31 | TRAC | TRBV29-1 | TRBD1 | TRBJ1-1 | TRBC1 |
| TCR445 | EVDPIGHLY | A0101 | TRAV39 | TRAJ33 | TRAC | TRBV6-1 | None | TRBJ1-5 | TRBC1 |
| TCR446 | EVDPIGHLY | A0101 | TRAV3 | TRAJ38 | TRAC | TRBV27 | TRBD2 | TRBJ2-7 | TRBC2 |
| TCR447 | EVDPIGHLY | A0101 | TRAV10 | TRAJ33 | TRAC | TRBV30 | TRBD2 | TRBJ2-1 | TRBC2 |
| TCR448 | EVDPIGHLY | A0101 | TRAV21 | TRAJ20 | TRAC | TRBV2 | TRBD1 | TRBJ2-1 | TRBC2 |
| TCR449 | EVDPIGHLY | A0101 | TRAV13-1 | TRAJ20 | TRAC | TRBV5-1 | TRBD1 | TRBJ1-1 | TRBC1 |
| TCR450 | EVDPIGHLY | A0101 | TRAV27 | TRAJ45 | TRAC | TRBV27 | TRBD1 | TRBJ1-6 | TRBC1 |
| TCR451 | EVDPIGHLY | A0101 | TRAV21 | TRAJ18 | TRAC | TRBV9 | TRBD1 | TRBJ2-1 | TRBC2 |
| TCR452 | EVDPIGHLY | A0101 | TRAV26-2 | TRAJ28 | TRAC | TRBV27 | None | TRBJ1-5 | TRBC1 |
| TCR453 | EVDPIGHLY | A0101 | TRAV12-1 | TRAJ34 | TRAC | TRBV9 | TRBD2 | TRBJ2-7 | TRBC2 |
| TCR454 | EVDPIGHLY | A0101 | TRAV13-2 | TRAJ40 | TRAC | TRBV4-1 | None | TRBJ1-3 | TRBC1 |
| TCR455 | EVDPIGHLY | A0101 | TRAV12-1 | TRAJ34 | TRAC | TRBV4-2 | TRBD2 | TRBJ2-7 | TRBC2 |
| TCR456 | EVDPIGHLY | A0101 | TRAV13-2 | TRAJ46 | TRAC | TRBV7-9 | TRBD1 | TRBJ2-1 | TRBC2 |
| TCR457 | EVDPIGHLY | A0101 | TRAV21 | TRAJ36 | TRAC | TRBV9 | TRBD2 | TRBJ2-7 | TRBC2 |
| TCR458 | EVDPIGHLY | A0101 | TRAV1-2 | TRAJ20 | TRAC | TRBV11-3 | TRBD1 | TRBJ2-3 | TRBC2 |
| TCR459 | EVDPIGHLY | A0101 | TRAV3 | TRAJ6 | TRAC | TRBV12-4 | TRBD1 | TRBJ2-2 | TRBC2 |
| TCR460 | EVDPIGHLY | A0101 | TRAV25 | TRAJ32 | TRAC | TRBV19 | TRBD1 | TRBJ1-1 | TRBC1 |
| TCR461 | EVDPIGHLY | A0101 | TRAV21 | TRAJ33 | TRAC | TRBV9 | TRBD1 | TRBJ1-1 | TRBC1 |
| TCR462 | EVDPIGHLY | A0101 | TRAV19 | TRAJ53 | TRAC | TRBV7-7 | TRBD1 | TRBJ2-1 | TRBC2 |
| TCR463 | EVDPIGHLY | A0101 | TRAV12-1 | TRAJ20 | TRAC | TRBV10-3 | TRBD2 | TRBJ2-3 | TRBC2 |
| TCR464 | EVDPIGHLY | A0101 | TRAV12-1 | TRAJ34 | TRAC | TRBV6-5 | TRBD1 | TRBJ2-7 | TRBC2 |
| TCR465 | EVDPIGHLY | A0101 | TRAV26-2 | TRAJ43 | TRAC | TRBV25-1 | TRBD1 | TRBJ1-2 | TRBC1 |
| TCR466 | EVDPIGHLY | A0101 | TRAV8-6 | TRAJ20 | TRAC | TRBV7-9 | TRBD1 | TRBJ2-2 | TRBC2 |
| TCR467 | EVDPIGHLY | A0101 | TRAV3 | TRAJ18 | TRAC | TRBV20-1 | TRBD2 | TRBJ2-1 | TRBC2 |
| TCR468 | EVDPIGHLY | A0101 | TRAV21 | TRAJ40 | TRAC | TRBV11-3 | TRBD1 | TRBJ1-2 | TRBC1 |
| TCR469 | EVDPIGHLY | A0101 | TRAV2 | TRAJ10 | TRAC | TRBV6-5 | TRBD2 | TRBJ2-7 | TRBC2 |

V(D)J and CDR3 sequences of a and 3 chains of the TCR clonotypes specific for A*0101_EVDPHIGHLY are shown in Table 10.

Annotated variable, diversity, joining, and constant regions of TCR clonotypes that demonstrated confirmed specificity in recipient T-cells is shown in Table 11, below.

TABLE 11
annotated TCR sequences from unique TCRs with confirmed specificity in
recipient T cells.

| TCR | | | | | | | | | |
|---|---|---|---|---|---|---|---|---|---|
| Clonotype | | | | | | | | | |
| ID # | PEPTIDE | HLA | TRAV | TRAJ | TRAC | TRBV | TRBD | TRBJ | TRBC |
| TCR2 | EVDPIGHLY | A0101 | TRAV24 | TRAJ31 | TRAC | TRBV3-1 | TRBD1 | TRBJ2-1 | TRBC |
| | | | | | | | | | 2 |
| TCR4 | EVDPIGHLY | A0101 | TRAV3 | TRAJ6 | TRAC | TRBV19 | None | TRBJ2-1 | TRBC |
| | | | | | | | | | 1 |
| TCR53 | EVDPIGHLY | A0101 | TRAV21 | TRAJ26 | TRAC | TRBV27 | TRBD1 | TRBJ1-6 | TRBC |
| | | | | | | | | | 1 |
| TCR54 | EVDPIGHLY | A0101 | TRAV20 | TRAJ15 | TRAC | TRBV27 | None | TRBJ2-3 | TRBC |
| | | | | | | | | | 2 |
| TCR19 | LLASSILCA | A0201 | TRAV19 | TRAJ4 | TRAC | TRBV6-5 | TRBD2 | TRBJ2-7 | TRBC |
| | | | | | | | | | 1 |
| TCR21 | LLASSILCA | A0201 | TRAV5 | TRAJ13 | TRAC | TRBV7-9 | TRBD1 | TRBJ2-7 | TRBC |
| | | | | | | | | | 2 |
| TCR22 | LLASSILCA | A0201 | TRAV3 | TRAJ39 | TRAC | TRBV7-9 | None | TRBJ2-2 | TRBC |
| | | | | | | | | | 2 |
| TCR18 | LLASSILCA | A0201 | TRAV38- | TRAJ21 | TRAC | TRBV9 | TRBD1 | TRBJ2-1 | TRBC |
| | | | 2DV8 | | | | | | 2 |
| TCR23 | LLASSILCA | A0201 | TRAV4 | TRAJ9 | TRAC | TRBV27 | None | TRBJ1-5 | TRBC |
| | | | | | | | | | 1 |
| TCR26 | GVYDGEEHSV | A0201 | TRAV13-1 | TRAJ11 | TRAC | TRBV6-3 | None | TRBJ2-1 | TRBC |
| | | | | | | | | | 2 |
| TCR28 | GVYDGEEHSV | A0201 | TRAV14DV4 | TRAJ54 | TRAC | TRBV4-3 | TRBD1 | TRBJ2-4 | TRBC |
| | | | | | | | | | 2 |
| TCR29 | GEMSSNSTAL | B4402 | TRAV19 | TRAJ39 | TRAC | TRBV7-6 | TRBD1 | TRBJ1-1 | TRBC |
| | | | | | | | | | 1 |
| TCR30 | GEMSSNSTAL | B4402 | TRAV36DV7 | TRAJ34 | TRAC | TRBV7-6 | TRBD2 | TRBJ2-2 | TRBC |
| | | | | | | | | | 2 |
| TCR32 | GEMSSNSTAL | B4402 | TRAV24 | TRAJ15 | TRAC | TRBV7-6 | TRBD2 | TRBJ2-1 | TRBC |
| | | | | | | | | | 2 |
| TCR33 | GEMSSNSTAL | B4402 | TRAV8-4 | TRAJ12 | TRAC | TRBV12- | TRBD2 | TRBJ2-3 | TRBC |
| | | | | | | 4 | | | 2 |

V(D)J and CDR3 sequences of α and β chains of TCR clonotypes that demonstrated confirmed specificity in recipient T-cells is shown in Table 12.

A table of the annotated reference a variable (TRAV), α joining (TRAJ), α constant (TRAC), β variable (TRBV), β diversity (TRBD), β joining (TRBJ), and β constant (TRBC) sequences and their corresponding Ensembl transcript (ENST) reference number is shown in Table 13. For any of the TCRs disclosed, amino acid sequences that are at least 95%, at least 96%, at least 97%, and least 98%, at least 99%, or more than 99% identical to the the annotated reference sequences as disclosed in Tables 9 and 11 are encompassed in the invention.

TABLE 13
Annotated reference genes for alpha and beta TCR regions

| Gene | Ensemble Transcript ID |
|---|---|
| TRAC | ENST00000636588, ENST00000637010, ENST00000611116, |
| | ENST00000636320, ENST00000616778 |
| TRAJ1 | ENST00000390536 |
| TRAJ10 | ENST00000390527 |
| TRAJ11 | ENST00000390526 |
| TRAJ12 | ENST00000390525 |
| TRAJ13 | ENST00000390524 |
| TRAJ14 | ENST00000390523 |
| TRAJ15 | TENX_TRAJ15 (10X internal ref #) |
| TRAJ16 | ENST00000390521 |
| TRAJ17 | ENST00000390520 |
| TRAJ18 | ENST00000390519 |
| TRAJ19 | ENST00000390518 |
| TRAJ2 | ENST00000390535 |
| TRAJ20 | ENST00000390517 |
| TRAJ21 | ENST00000390516 |
| TRAJ22 | ENST00000390515 |
| TRAJ23 | ENST00000390514 |
| TRAJ24 | ENST00000390513 |
| TRAJ25 | ENST00000390512 |
| TRAJ26 | ENST00000390511 |
| TRAJ27 | ENST00000390510 |
| TRAJ28 | ENST00000390509 |
| TRAJ29 | ENST00000390508 |
| TRAJ3 | ENST00000390534 |
| TRAJ30 | ENST00000390507 |
| TRAJ31 | ENST00000390506 |
| TRAJ32 | ENST00000390505 |
| TRAJ33 | ENST00000390504 |
| TRAJ34 | ENST00000390503 |
| TRAJ35 | ENST00000390502 |
| TRAJ36 | ENST00000614481 |
| TRAJ37 | ENST00000612375 |
| TRAJ38 | ENST00000390499 |
| TRAJ39 | ENST00000390498 |
| TRAJ4 | ENST00000390533 |
| TRAJ40 | ENST00000390497 |
| TRAJ41 | ENST00000390496 |
| TRAJ42 | ENST00000390495 |
| TRAJ43 | ENST00000390494 |
| TRAJ44 | ENST00000390493 |
| TRAJ45 | ENST00000390492 |
| TRAJ46 | ENST00000390491 |
| TRAJ47 | ENST00000390490 |
| TRAJ48 | ENST00000390489 |
| TRAJ49 | ENST00000390488 |
| TRAJ5 | ENST00000390532 |
| TRAJ50 | ENST00000390487 |
| TRAJ52 | ENST00000390486 |
| TRAJ53 | ENST00000390485 |
| TRAJ54 | ENST00000390484 |
| TRAJ56 | ENST00000390483 |
| TRAJ57 | ENST00000390482 |
| TRAJ58 | ENST00000390481 |
| TRAJ59 | ENST00000390480 |
| TRAJ6 | ENST00000390531 |
| TRAJ61 | ENST00000390479 |
| TRAJ7 | ENST00000390530 |
| TRAJ8 | ENST00000390529 |
| TRAJ9 | ENST00000390528 |
| TRAV1-1 | ENST00000542354 |
| TRAV1-2 | ENST00000390423 |
| TRAV10 | ENST00000390432 |
| TRAV12-1 | ENST00000390433 |
| TRAV12-2 | ENST00000390437 |
| TRAV12-3 | ENST00000390442 |
| TRAV13-1 | ENST00000390436 |
| TRAV13-2 | ENST00000390439 |
| TRAV14DV4 | ENST00000390440 |
| TRAV16 | ENST00000390444 |
| TRAV17 | ENST00000390445 |
| TRAV18 | ENST00000390446 |
| TRAV19 | ENST00000390447 |
| TRAV2 | ENST00000390424 |
| TRAV20 | ENST00000390448 |
| TRAV21 | ENST00000390449 |
| TRAV22 | ENST00000390450 |
| TRAV23DV6 | ENST00000390451 |
| TRAV24 | ENST00000390453 |
| TRAV25 | ENST00000390454 |
| TRAV26-1 | ENST00000390455 |
| TRAV26-2 | ENST00000390460 |
| TRAV27 | ENST00000390457 |
| TRAV29DV5 | ENST00000390458 |
| TRAV3 | ENST00000390425 |
| TRAV30 | ENST00000557168 |
| TRAV34 | ENST00000390461 |
| TRAV35 | TENX_TRAV35 (10X internal ref #) |
| TRAV36DV7 | ENST00000390463 |
| TRAV38-1 | ENST00000390464 |
| TRAV38-2DV8 | ENST00000390465 |
| TRAV39 | ENST00000390466 |
| TRAV4 | ENST00000390426 |
| TRAV40 | ENST00000390467 |
| TRAV41 | ENST00000390468 |
| TRAV5 | ENST00000390427 |
| TRAV6 | ENST00000390428 |
| TRAV7 | ENST00000390429 |
| TRAV8-1 | ENST00000390430 |
| TRAV8-2 | ENST00000390434 |
| TRAV8-3 | ENST00000390435 |
| TRAV8-4 | ENST00000390438 |
| TRAV8-6 | ENST00000390443 |
| TRAV8-7 | ENST00000390456 |
| TRAV9-1 | ENST00000390431 |
| TRAV9-2 | ENST00000390441 |
| TRBC1 | ENST00000632136, ENST00000633705 |
| TRBC2 | ENST00000636844, ENST00000614992, ENST00000622053, |
| | ENST00000613720, ENST00000466254, ENST00000637077, |
| | ENST00000610416, ENST00000620987 |
| TRBD1 | ENST00000631435 |
| TRBD2 | TENX_TRBD2 (10X internal ref #) |
| TRBJ1-1 | ENST00000632951 |
| TRBJ1-2 | ENST00000631745 |
| TRBJ1-3 | ENST00000633936 |
| TRBJ1-4 | ENST00000632041 |
| TRBJ1-5 | ENST00000634000 |
| TRBJ1-6 | ENST00000633713, ENST00000632228 |
| TRBJ2-1 | ENST00000631600 |
| TRBJ2-2 | ENST00000633188 |
| TRBJ2-2P | ENST00000633209 |
| TRBJ2-3 | ENST00000631840 |
| TRBJ2-4 | ENST00000390416 |
| TRBJ2-5 | ENST00000634149 |
| TRBJ2-6 | ENST00000632996 |
| TRBJ2-7 | ENST00000390419, ENST00000633660 |
| TRBV10-1 | ENST00000390364 |
| TRBV10-2 | ENST00000426318, ENST00000633575 |
| TRBV10-3 | ENST00000611462, ENST00000631471 |
| TRBV11-1 | ENST00000390367 |
| TRBV11-2 | TENX_TRBV11_2 (10X internal ref #) |
| TRBV11-3 | ENST00000634111 |
| TRBV12-3 | ENST00000633292 |
| TRBV12-4 | ENST00000631824, ENST00000617347 |
| TRBV12-5 | ENST00000632829, ENST00000621184 |
| TRBV13 | ENST00000633796 |
| TRBV14 | ENST00000617639 |
| TRBV15 | ENST00000631835 |
| TRBV16 | ENST00000633244 |
| TRBV17 | ENST00000619103, ENST00000631663 |
| TRBV18 | ENST00000611520, ENST00000631559 |
| TRBV19 | ENST00000390393, ENST00000632638 |
| TRBV2 | ENST00000632828, ENST00000455382 |
| TRBV20-1 | ENST00000390394, ENST00000633466 |
| TRBV21-1 | TENXT_RBV21 (10X internal ref #) |
| TRBV23-1 | ENST00000390396 |
| TRBV24-1 | ENST00000633092, ENST00000390397 |
| TRBV25-1 | ENST00000390398, ENST00000610439 |
| TRBV27 | ENST00000633283 |
| TRBV28 | ENST00000390400 |
| TRBV29-1 | ENST00000422143 |
| TRBV3-1 | ENST00000390387 |
| TRBV30 | ENST00000631690, ENST00000417977 |
| TRBV4-1 | ENST00000632713, ENST00000390357 |
| TRBV4-2 | ENST00000390392 |
| TRBV4-3 | ENST00000631427 |
| TRBV5-1 | ENST00000633384 |
| TRBV5-3 | ENST00000390362, ENST00000634123 |
| TRBV5-4 | ENST00000633696, ENST00000454561 |
| TRBV5-5 | ENST00000632187, ENST00000390372 |
| TRBV5-6 | ENST00000390375 |
| TRBV5-7 | ENST00000633790 |
| TRBV5-8 | ENST00000631639 |
| TRBV6-1 | ENST00000631557 |
| TRBV6-2 | ENST00000632016 |
| TRBV6-3 | ENST00000632148 |
| TRBV6-4 | ENST00000390360, ENST00000633472 |
| TRBV6-5 | ENST00000633072 |
| TRBV6-6 | ENST00000633963, ENST00000390371 |
| TRBV6-7 | ENST00000631511, ENST00000390373 |
| TRBV6-8 | ENST00000632425, ENST00000390376 |
| TRBV6-9 | ENST00000634093 |
| TRBV7-1 | ENST00000632308 |
| TRBV7-2 | ENST00000634605 |
| TRBV7-3 | ENST00000390361, ENST00000631882 |
| TRBV7-4 | ENST00000633313, ENST00000390359 |
| TRBV7-6 | ENST00000390374, ENST00000633265 |
| TRBV7-7 | ENST00000631548 |
| TRBV7-8 | ENST00000632560 |
| TRBV7-9 | ENST00000612787, ENST00000632021 |
| TRBV9 | ENST00000633328, ENST00000390363 |

### Example 13: T Cell Line Transiently Transfected with Identified TCRs Specifically Bind to their Target HLA-PEPTIDE Complex, but not to Negative Control Peptide-HLAs

Jurkat TIB-152 T cell line cultures were co-transfected with a plasmid expressing human CD8 and a plasmid expressing TCR α and β chains with a GFP reporter gene using Nucleofector 4D electroporator. Plasmids used for transfection are described in FIGS. 4 and 5. 24-48 hours post transfection, Jurkat T cells were analyzed for expression of the TCR of interest. Cells were assessed for binding to HLA-PEPTIDE complexes and a control infectious-disease-based peptide tetramer using flow cytometry. Total population was gated on live single GFP-expressing cells before evaluating binding of HLA-PEPTIDE target tetramer. FIG. 31 shows examples of Jurkat cells expressing A*0201_LLASSILCA-, A*0201_GVYDGEEHSV-, B*4402_GEMSSNSTAL-, and A*0101_EVDPIGHLY-specific TCRs binding to their respective HLA-PEPTIDE targets but not to the control peptide tetramer.

### Example 14: TCRs Cloned into a Viral Vector are Stably Expressed in Primary Human CD8+ T Cells and Bind Cognate Peptide Target-MHC Complexes

Lentiviral vectors were generated for TCR specific for the HLA-PEPTIDE target HLA-A*0201_LLASSILCA. As a model vector system, we used commercially available 3^{rd} generation lentivirus base expression vector system from System Biosciences, Palo Alto, Calif. See FIG. 33.

Primary human CD8+ T cells were isolated and transduced with the recombinant TCR lentivirus at multiplicity of infection (MOI˜10). T cells were expanded using rapid expansion protocol for 1-2 weeks before assessment of TCR expression on CD8 T cells by tetramer staining.

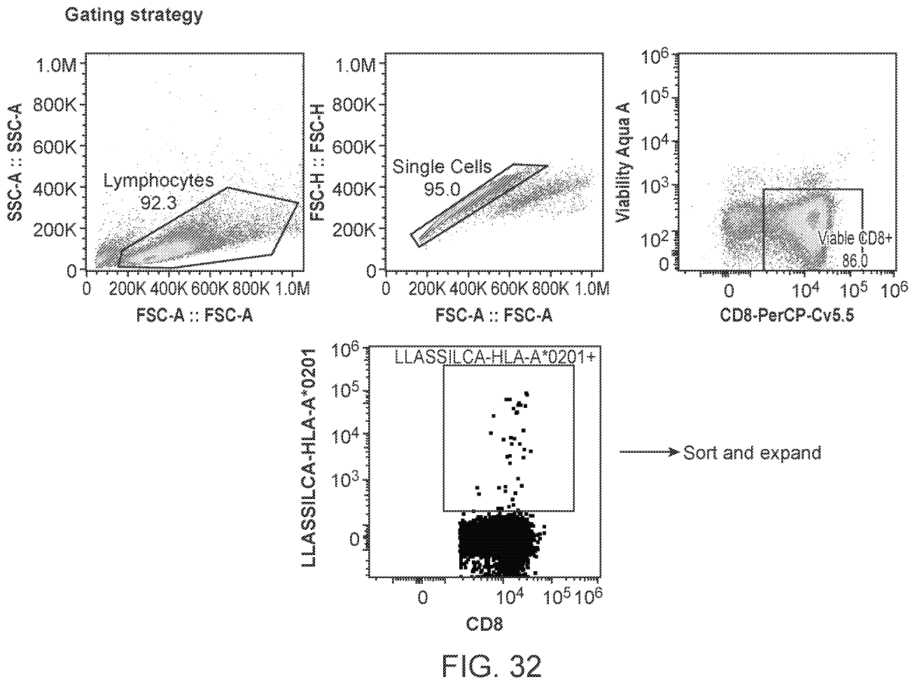

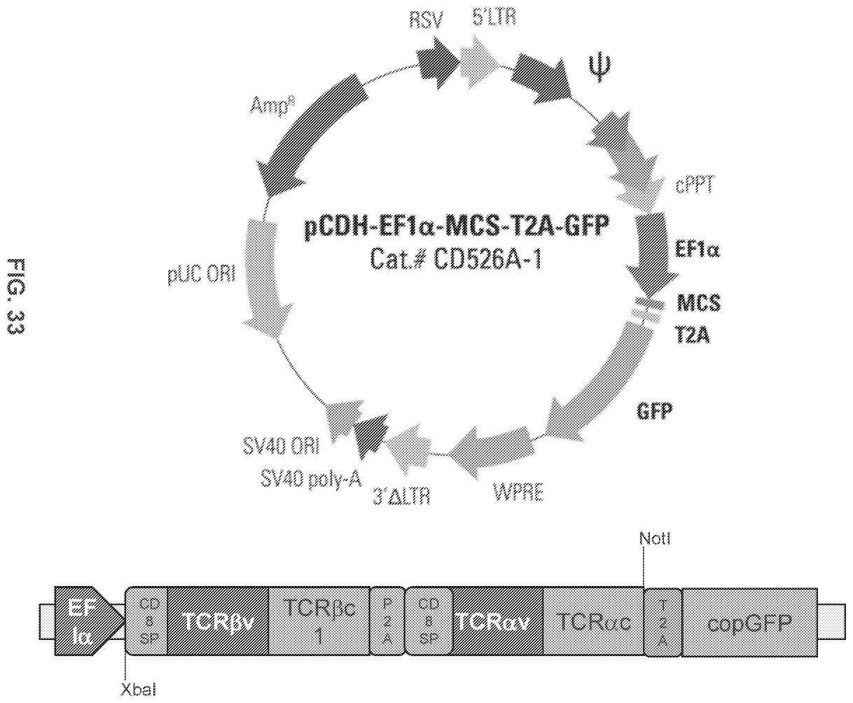

FIG. 32 depicts the gating strategy and flow data demonstrating that transduced human CD8+ cells bind to the HLA-PEPTIDE target.

### Example 15: Identification of MHC/Peptide Target-Reactive TCRs

T cells are isolated from blood, lymph nodes, or tumors of patients. Patients are HLA-matched to SAT, and are selected based on expression of target-harboring protein. T cells are then enriched for SAT-specific T cells, e.g., by sorting SAT-MHC tetramer binding cells or by sorting activated cells stimulated in an in vitro co-culture of T cells and SAT-pulsed antigen presenting cells.

SAT-relevant alpha-beta TCR dimers are identified by single cell sequencing of TCRs of SAT-specific T cells. Alternatively, bulk TCR sequencing of SAT-specific T cells is performed and alpha-beta pairs with a high probability of matching are determined using a TCR pairing method.

Alternatively or in addition, SAT-specific T cells can be obtained through in vitro priming of naïve T cells from healthy donors. T cells obtained from PBMCs, lymph nodes, or cord blood are repeatedly stimulated by SAT-pulsed antigen presenting cells to prime differentiation of antigen-experienced T cells. TCRs are then identified similarly as described above for SAT-specific T cells from patients.

### Example 16: Production of Engineered TCR T Cells

TCR alpha and beta chain sequences are cloned into appropriate constructs. TCR-autologous or heterologous bulk T cells are transduced with the constructs to produce engineered TCR T cells. These T cells are expanded in the presence of anti-CD3 antibodies and IL-2 cytokine for use in subsequent experiments. In certain instances, native TCR is deleted or the inserted TCR is modified to increase proper multimerization.

In Vitro Verification of TCR Specificity

First, T cells bearing engineered TCRs are screened for target recognition using antigen presenting cells expressing the appropriate MHC and pulsed with appropriate target(s).

TCRs identified in the first round of screening are then tested for recognition of natural target. Lead TCRs are nominated based on specific recognition of HLA-matched primary tumors and tumor cell lines expressing SAT-harboring protein.

To assure specificity, lead TCRs are de-selected based on off-target recognition. They are screened against a panel of HLA matched and mismatched cell lines, covering multiple tissues and organ types, and with HLA-matched and mismatched antigen presenting cells pulsed with a panel of infectious disease antigens. TCRs with specific and non-specific off-target recognition of self-antigens or common non-self-antigens are de-selected.

### Example 17: Identification of Monoclonal Antibodies (mAbs) that Target MHC Class I Molecules Presenting Tumor Antigens Using Rabbit B Cell Cloning Technologies

Potent and selective mAbs targeting human class I MHC molecules presenting tumor antigens of interest are identified. Soluble human pMHC molecules presenting human tumor antigens are utilized for multiple mouse or rabbit immunizations followed by screening of B cells derived from the immunized animals to identify B cells that express mAbs that bind to target class I MHC molecules. Sequences encoding the mAbs identified from the mouse or rabbit screens will be cloned from the isolated B cells. The recovered mAbs are then evaluated against a panel of irrelevant pMHCs to identify lead mAbs that bind selectively to the target pMHCs. Lead mAbs will be fully characterized to determine target binding affinity and selectivity. Lead mAbs that demonstrate potent and selective binding are humanized to generate full-length human IgG monoclonal antibody (mAb) constructs. In addition, the lead mAbs are incorporated into bi-specific mAb constructs and chimeric antigen receptor (CAR) constructs that can be used to generate CAR T-cells. Full-length bi-specifics or scFV-based bi-specifics can be constructed.

Demonstrate Targeting of Human Tumor Cells In Vitro

Immunohistochemistry techniques are utilized to demonstrate specific binding of lead antibodies to human tumor cells expressing target pMHC molecules. T-cell lines transfected with CAR-T constructs are incubated with human tumor cells to demonstrate killing of tumor cells in vitro. Alternatively, tumor cells expressing the target are incubated with bi-specific constructs (encoding the ABP and an effector domain) and PBMCs or T cells.

In Vivo Proof-of-Concept

Lead antibody or CAR-T constructs are evaluated in vivo to demonstrate directed tumor killing in humanized mouse tumor models. Lead antibody or CAR-T constructs are evaluated in xenograft tumor models engrafted with human PBMCs. Anti-tumor activity is measured and compared to control constructs to demonstrate target-dependent tumor killing.

Potent and selective ABPs that selectively target human class I MHC molecules presenting tumor antigens will be identified using phage display or B cell cloning technologies. The utility of the ABPs will be demonstrated by showing that the ABPs mediated tumor cell killing in vitro and in vivo when incorporated into antibody or CAR-T cell constructs.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### SEQUENCES

TABLE 3
VH and VL sequences for G2 scFv Selective
Binders, selective for HLA-PEPTIDE Target
HLA-A*01:01 NTDNNLAVY (SEQ ID NO: 23).

| Target group | Clone name | VH (SEQ ID NO) | VL (SEQ ID NO) |
|---|---|---|---|
| G2 | G2-P2E07 | 2781 | 2816 |
| G2 | G2-P2E03 | 2782 | 2817 |
| G2 | G2-P2A11 | 2783 | 2818 |
| G2 | G2-P2C06 | 2784 | 2819 |
| G2 | G2-P1G01 | 2785 | 2820 |
| G2 | G2-P1C02 | 2786 | 2821 |
| G2 | G2-P1H01 | 2787 | 2822 |
| G2 | G2-P1B12 | 2788 | 2823 |
| G2 | G2-P1B06 | 2789 | 2824 |
| G2 | G2-P2H10 | 2790 | 2825 |
| G2 | G2-P1H10 | 2791 | 2826 |
| G2 | G2-P2C11 | 2792 | 2827 |
| G2 | G2-P1C09 | 2793 | 2828 |
| G2 | G2-P1A10 | 2794 | 2829 |
| G2 | G2-P1B10 | 2795 | 2830 |
| G2 | G2-P1D07 | 2796 | 2831 |
| G2 | G2-P1E05 | 2797 | 2832 |
| G2 | G2-P1D03 | 2798 | 2833 |
| G2 | G2-P1G12 | 2799 | 2834 |
| G2 | G2-P2H11 | 2800 | 2835 |
| G2 | G2-P1C03 | 2801 | 2836 |
| G2 | G2-P1G07 | 2802 | 2837 |
| G2 | G2-P1F12 | 2803 | 2838 |
| G2 | G2-P1G03 | 2804 | 2839 |
| G2 | G2-P2B08 | 2805 | 2840 |
| G2 | G2-P2A10 | 2806 | 2841 |
| G2 | G2-P2D04 | 2807 | 2842 |
| G2 | G2-P1C06 | 2808 | 2843 |
| G2 | G2-P2A09 | 2809 | 2844 |
| G2 | G2-P1B08 | 2810 | 2845 |
| G2 | G2-P1E03 | 2811 | 2846 |
| G2 | G2-P2A03 | 2812 | 2847 |
| G2 | G2-P2F01 | 2813 | 2848 |
| G2 | G2-P1H11 | 2814 | 2849 |
| G2 | G2-P1D06 | 2815 | 2850 |

TABLE 4
CDR sequences for G2 selective binders, selective for HLA-PEPTIDE
Target HLA-A*01:01 NTDNNLAVY (SEQ ID NO: 23)
(determined according to Kabat numbering)

| | | CDR- | CDR- | CDR- | CDR- | CDR- | CDR- |
|---|---|---|---|---|---|---|---|
| Tar- | | H1 | H2 | H3 | L1 | L2 | L3 |
| get | Clone | (SEQ | (SEQ | (SEQ | (SEQ | (SEQ | (SEQ |
| group | name | ID NO) | ID NO) | ID NO) | ID NO) | ID NO) | ID NO) |
| G2 | G2-P2E07 | 2851 | 2880 | 2902 | 2934 | 2955 | 2971 |
| G2 | G2-P2E03 | 2852 | 2881 | 2903 | 2935 | 2956 | 2972 |
| G2 | G2-P2A11 | 2853 | 2882 | 2903 | 2936 | 2957 | 2973 |
| G2 | G2-P2C06 | 2854 | 2882 | 2904 | 2937 | 2958 | 2974 |
| G2 | G2-P1G01 | 2855 | 2883 | 2905 | 2937 | 2958 | 2975 |
| G2 | G2-P1C02 | 2855 | 2882 | 2906 | 2938 | 2958 | 2976 |
| G2 | G2-P1H01 | 2856 | 2882 | 2907 | 2939 | 2959 | 2976 |
| G2 | G2-P1B12 | 2857 | 2882 | 2908 | 2940 | 2960 | 2977 |
| G2 | G2-P1B06 | 2858 | 2884 | 2909 | 2935 | 2958 | 2972 |
| G2 | G2-P2H10 | 2859 | 2882 | 2910 | 2941 | 2961 | 2978 |
| G2 | G2-P1H10 | 2852 | 2885 | 2911 | 2942 | 2958 | 2976 |
| G2 | G2-P2C11 | 2860 | 2882 | 2912 | 2943 | 2962 | 2978 |
| G2 | G2-P1C09 | 2861 | 2886 | 2913 | 2944 | 2963 | 2979 |
| G2 | G2-P1A10 | 2862 | 2887 | 2914 | 2945 | 2958 | 2980 |
| G2 | G2-P1B10 | 2855 | 2888 | 2903 | 2941 | 2962 | 2981 |
| G2 | G2-P1D07 | 2855 | 2889 | 2915 | 2946 | 2958 | 2982 |
| G2 | G2-P1E05 | 2863 | 2883 | 2916 | 2947 | 2958 | 2973 |
| G2 | G2-P1D03 | 2856 | 2890 | 2917 | 2934 | 2962 | 2972 |
| G2 | G2-P1G12 | 2864 | 2891 | 2917 | 2946 | 2964 | 2972 |
| G2 | G2-P2H11 | 2865 | 2882 | 2918 | 2941 | 2962 | 2974 |
| G2 | G2-P1C03 | 2866 | 2882 | 2919 | 2948 | 2958 | 2983 |
| G2 | G2-P1G07 | 2867 | 2892 | 2920 | 2946 | 2962 | 2984 |
| G2 | G2-P1F12 | 2868 | 2893 | 2921 | 2949 | 2965 | 2972 |
| G2 | G2-P1G03 | 2869 | 2894 | 2922 | 2950 | 2966 | 2985 |
| G2 | G2-P2B08 | 2870 | 2882 | 2923 | 2943 | 2967 | 2986 |
| G2 | G2-P2A10 | 2871 | 2895 | 2924 | 2951 | 2968 | 2987 |
| G2 | G2-P2D04 | 2872 | 2882 | 2925 | 2952 | 2969 | 2973 |
| G2 | G2-P1C06 | 2873 | 2882 | 2926 | 2943 | 2958 | 2988 |
| G2 | G2-P2A09 | 2852 | 2882 | 2927 | 2935 | 2958 | 2989 |
| G2 | G2-P1B08 | 2874 | 2896 | 2928 | 2938 | 2958 | 2981 |
| G2 | G2-P1E03 | 2875 | 2897 | 2929 | 2953 | 2961 | 2990 |
| G2 | G2-P2A03 | 2876 | 2898 | 2930 | 2941 | 2962 | 2989 |
| G2 | G2-P2F01 | 2877 | 2899 | 2931 | 2946 | 2964 | 2991 |
| G2 | G2-P1H11 | 2878 | 2900 | 2932 | 2946 | 2958 | 2992 |
| G2 | G2-P1D06 | 2879 | 2901 | 2933 | 2954 | 2970 | 2993 |

TABLE 5
VH and VL sequences for scFv selective binders selective for
HLA-PEPTIDE Target HLA-A*02:01 LLASSILCA (SEQ ID NO: 2737).

| Target group | Clone name | VH (SEQ ID NO) | VL (SEQ ID NO) |
|---|---|---|---|
| G7 | G7R3-P1C6 | 2994 | 3002 |
| G7 | G7R3-P1G10 | 2995 | 3003 |
| G7 | 1-G7R3-P1B4 | 2996 | 3004 |
| G7 | 2-G7R4-P2C2 | 2997 | 3005 |
| G7 | 3-G7R4-P1A3 | 2998 | 3006 |
| G7 | 4-G7R4-B5-P2E9 | 2999 | 3007 |
| G7 | 5-G7R4-B10-P1F8 | 3000 | 3008 |
| G7 | B7 (G7R3-P3A9) | 3001 | 3009 |

TABLE 6
CDR sequences for G7 selective binders selective for HLA-PEPTIDE
Target HLA-A*02:01 LLASSILCA (SEQ ID NO: 2737)

| | | CDR- | CDR- | CDR- | CDR- | CDR- | CDR- |
|---|---|---|---|---|---|---|---|
| Tar- | | H1 | H2 | H3 | L1 | L2 | L3 |
| get | Clone | (SEQ | (SEQ | (SEQ | (SEQ | (SEQ | (SEQ |
| group | name | ID NO) | ID NO) | ID NO) | ID NO) | ID NO) | ID NO) |
| G7 | G7R3- | 3010 | 3017 | 3025 | 3033 | 2970 | 3043 |
| | P1C6 | | | | | | |
| G7 | G7R3- | 3011 | 3018 | 3026 | 3034 | 2958 | 3044 |
| | P1G10 | | | | | | |
| G7 | 1-G7R3- | 3012 | 3019 | 3027 | 3035 | 3039 | 3045 |
| | P1B4 | | | | | | |
| G7 | 2-G7R4- | 3013 | 3020 | 3028 | 3036 | 2962 | 3046 |
| | P2C2 | | | | | | |
| G7 | 3-G7R4- | 2879 | 3021 | 3029 | 2934 | 3040 | 3047 |
| | P1A3 | | | | | | |
| G7 | 4-G7R4- | 3014 | 3022 | 3030 | 3037 | 3041 | 3048 |
| | B5-P2E9 | | | | | | |
| G7 | 5-G7R4- | 3015 | 3023 | 3031 | 2946 | 3042 | 3049 |
| | B10-P1F8 | | | | | | |
| G7 | B7 | 3016 | 3024 | 3032 | 3038 | 3041 | 3050 |
| | (G7R3- | | | | | | |
| | P3 A9) | | | | | | |

CDR3 and V(D)J sequences of TCR clonotypes confirmed through resorting

| | | | ALPHA | | FULL LENGTH | FULL LENGTH |
|---|---|---|---|---|---|---|
| TCR ID | | | CDR3 (SEQ | BETA CDR3 | ALPHA VJ (SEQ | BETA V(D)J |
| # | PEPTIDE | HLA | ID NO) | (SEQ ID NO) | ID NO) | (SEQ ID NO) |
| TCR101 | EVDPIGHLY (SEQ | A0101 | 3052 | 3351 | 3656 | 3962 |
| | ID NO: 3051) | | | | | |
| TCR102 | EVDPIGHLY (SEQ | A0101 | 3053 | 3352 | 3657 | 3963 |
| | ID NO: 3051) | | | | | |
| TCR103 | EVDPIGHLY (SEQ | A0101 | 3054 | 3353 | 3658 | 3964 |
| | ID NO: 3051) | | | | | |
| TCR104 | EVDPIGHLY (SEQ | A0101 | 3052 | 3352 | 3659 | 3963 |
| | ID NO: 3051) | | | | | |
| TCR105 | EVDPIGHLY (SEQ | A0101 | 3055 | 3354 | 3660 | 3965 |
| | ID NO: 3051) | | | | | |
| TCR106 | EVDPIGHLY (SEQ | A0101 | 3056 | 3355 | 3661 | 3966 |
| | ID NO: 3051) | | | | | |
| TCR107 | EVDPIGHLY (SEQ | A0101 | 3057 | 3356 | 3662 | 3967 |
| | ID NO: 3051) | | | | | |
| TCR108 | EVDPIGHLY (SEQ | A0101 | 3058 | 3357 | 3663 | 3968 |
| | ID NO: 3051) | | | | | |
| TCR109 | EVDPIGHLY (SEQ | A0101 | 3059 | 3358 | 3664 | 3969 |
| | ID NO: 3051) | | | | | |
| TCR110 | EVDPIGHLY (SEQ | A0101 | 3060 | 3359 | 3665 | 3970 |
| | ID NO: 3051) | | | | | |
| TCR111 | EVDPIGHLY (SEQ | A0101 | 3061 | 3360 | 3666 | 3971 |
| | ID NO: 3051) | | | | | |
| TCR112 | EVDPIGHLY (SEQ | A0101 | 3062 | 3361 | 3667 | 3972 |
| | ID NO: 3051) | | | | | |
| TCR113 | EVDPIGHLY (SEQ | A0101 | 3063 | 3362 | 3668 | 3973 |
| | ID NO: 3051) | | | | | |
| TCR114 | EVDPIGHLY (SEQ | A0101 | 3053 | 3351 | 3657 | 3962 |
| | ID NO: 3051) | | | | | |
| TCR115 | EVDPIGHLY (SEQ | A0101 | 3057 | 3352 | 3662 | 3963 |
| | ID NO: 3051) | | | | | |
| TCR1I6 | EVDPIGHLY (SEQ | A0101 | 3064 | 3363 | 3669 | 3974 |
| | ID NO: 3051) | | | | | |
| TCR1I7 | EVDPIGHLY (SEQ | A0101 | 3065 | 3364 | 3670 | 3975 |
| | ID NO: 3051) | | | | | |
| TCR118 | EVDPIGHLY (SEQ | A0101 | 3054 | 3352 | 3658 | 3963 |
| | ID NO: 3051) | | | | | |
| TCR119 | EVDPIGHLY (SEQ | A0101 | 3066 | 3365 | 3671 | 3976 |
| | ID NO: 3051) | | | | | |
| TCR120 | EVDPIGHLY (SEQ | A0101 | 3067 | 3366 | 3672 | 3977 |
| | ID NO: 3051) | | | | | |
| TCR121 | EVDPIGHLY (SEQ | A0101 | 3068 | 3367 | 3673 | 3978 |
| | ID NO: 3051) | | | | | |
| TCR122 | EVDPIGHLY (SEQ | A0101 | 3069 | 3368 | 3674 | 3979 |
| | ID NO: 3051) | | | | | |
| TCR123 | EVDPIGHLY (SEQ | A0101 | 3052 | 3356 | 3659 | 3967 |
| | ID NO: 3051) | | | | | |
| TCR124 | EVDPIGHLY (SEQ | A0101 | 3070 | 3369 | 3675 | 3980 |
| | ID NO: 3051) | | | | | |
| TCR125 | EVDPIGHLY (SEQ | A0101 | 3052 | 3355 | 3659 | 3966 |
| | ID NO: 3051) | | | | | |
| TCR126 | EVDPIGHLY (SEQ | A0101 | 3071 | 3370 | 3676 | 3981 |
| | ID NO: 3051) | | | | | |
| TCR127 | EVDPIGHLY (SEQ | A0101 | 3052 | 3353 | 3659 | 3964 |
| | ID NO: 3051) | | | | | |
| TCR128 | EVDPIGHLY (SEQ | A0101 | 3072 | 3371 | 3677 | 3982 |
| | ID NO: 3051) | | | | | |
| TCR129 | EVDPIGHLY (SEQ | A0101 | 3073 | 3372 | 3678 | 3983 |
| | ID NO: 3051) | | | | | |
| TCR130 | EVDPIGHLY (SEQ | A0101 | 3057 | 3351 | 3662 | 3962 |
| | ID NO: 3051) | | | | | |
| TCR131 | EVDPIGHLY (SEQ | A0101 | 3074 | 3373 | 3679 | 3984 |
| | ID NO: 3051) | | | | | |
| TCR132 | EVDPIGHLY (SEQ | A0101 | 3075 | 3374 | 3680 | 3985 |
| | ID NO: 3051) | | | | | |
| TCR133 | EVDPIGHLY (SEQ | A0101 | 3076 | 3375 | 3681 | 3986 |
| | ID NO: 3051) | | | | | |
| TCR134 | EVDPIGHLY (SEQ | A0101 | 3077 | 3376 | 3682 | 3987 |
| | ID NO: 3051) | | | | | |
| TCR135 | EVDPIGHLY (SEQ | A0101 | 3078 | 3377 | 3683 | 3988 |
| | ID NO: 3051) | | | | | |
| TCR136 | EVDPIGHLY (SEQ | A0101 | 3079 | 3378 | 3684 | 3989 |
| | ID NO: 3051) | | | | | |
| TCR137 | EVDPIGHLY (SEQ | A0101 | 3080 | 3379 | 3685 | 3990 |
| | ID NO: 3051) | | | | | |
| TCR138 | EVDPIGHLY (SEQ | A0101 | 3081 | 3380 | 3686 | 3991 |
| | ID NO: 3051) | | | | | |
| TCR139 | EVDPIGHLY (SEQ | A0101 | 3082 | 3381 | 3687 | 3992 |
| | ID NO: 3051) | | | | | |
| TCR140 | EVDPIGHLY (SEQ | A0101 | 3083 | 3382 | 3688 | 3993 |
| | ID NO: 3051) | | | | | |
| TCR141 | EVDPIGHLY (SEQ | A0101 | 3084 | 3383 | 3689 | 3994 |
| | ID NO: 3051) | | | | | |
| TCR142 | EVDPIGHLY (SEQ | A0101 | 3085 | 3384 | 3690 | 3995 |
| | ID NO: 3051) | | | | | |
| TCR143 | EVDPIGHLY (SEQ | A0101 | 3086 | 3385 | 3691 | 3996 |
| | ID NO: 3051) | | | | | |
| TCR144 | EVDPIGHLY (SEQ | A0101 | 3087 | 3386 | 3692 | 3997 |
| | ID NO: 3051) | | | | | |
| TCR145 | EVDPIGHLY (SEQ | A0101 | 3088 | 3387 | 3693 | 3998 |
| | ID NO: 3051) | | | | | |
| TCR146 | EVDPIGHLY (SEQ | A0101 | 3089 | 3388 | 3694 | 3999 |
| | ID NO: 3051) | | | | | |
| TCR147 | EVDPIGHLY (SEQ | A0101 | 3052 | 3389 | 3695 | 4000 |
| | ID NO: 3051) | | | | | |
| TCR148 | EVDPIGHLY (SEQ | A0101 | 3056 | 3351 | 3661 | 3962 |
| | ID NO: 3051) | | | | | |
| TCR149 | EVDPIGHLY (SEQ | A0101 | 3090 | 3390 | 3696 | 4001 |
| | ID NO: 3051) | | | | | |
| TCR150 | EVDPIGHLY (SEQ | A0101 | 3091 | 3391 | 3697 | 4002 |
| | ID NO: 3051) | | | | | |
| TCR151 | EVDPIGHLY (SEQ | A0101 | 3092 | 3392 | 3698 | 4003 |
| | ID NO: 3051) | | | | | |
| TCR152 | EVDPIGHLY (SEQ | A0101 | 3093 | 3393 | 3699 | 4004 |
| | ID NO: 3051) | | | | | |
| TCR153 | EVDPIGHLY (SEQ | A0101 | 3053 | 3356 | 3700 | 3967 |
| | ID NO: 3051) | | | | | |
| TCR154 | EVDPIGHLY (SEQ | A0101 | 3094 | 3394 | 3701 | 4005 |
| | ID NO: 3051) | | | | | |
| TCR155 | EVDPIGHLY (SEQ | A0101 | 3054 | 3363 | 3658 | 3974 |
| | ID NO: 3051) | | | | | |
| TCR156 | EVDPIGHLY (SEQ | A0101 | 3095 | 3395 | 3702 | 4006 |
| | ID NO: 3051) | | | | | |
| TCR157 | EVDPIGHLY (SEQ | A0101 | 3054 | 3351 | 3658 | 3962 |
| | ID NO: 3051) | | | | | |
| TCR158 | EVDPIGHLY (SEQ | A0101 | 3096 | 3396 | 3703 | 4007 |
| | ID NO: 3051) | | | | | |
| TCR159 | EVDPIGHLY (SEQ | A0101 | 3053 | 3355 | 3657 | 3966 |
| | ID NO: 3051) | | | | | |
| TCR160 | EVDPIGHLY (SEQ | A0101 | 3097 | 3397 | 3704 | 4008 |
| | ID NO: 3051) | | | | | |
| TCR161 | EVDPIGHLY (SEQ | A0101 | 3098 | 3398 | 3705 | 4009 |
| | ID NO: 3051) | | | | | |
| TCR162 | EVDPIGHLY (SEQ | A0101 | 3099 | 3352 | 3706 | 3963 |
| | ID NO: 3051) | | | | | |
| TCR163 | EVDPIGHLY (SEQ | A0101 | 3100 | 3399 | 3707 | 4010 |
| | ID NO: 3051) | | | | | |
| TCR164 | EVDPIGHLY (SEQ | A0101 | 3053 | 3353 | 3657 | 3964 |
| | ID NO: 3051) | | | | | |
| TCR165 | EVDPIGHLY (SEQ | A0101 | 3101 | 3400 | 3708 | 4011 |
| | ID NO: 3051) | | | | | |
| TCR166 | EVDPIGHLY (SEQ | A0101 | 3102 | 3401 | 3709 | 4012 |
| | ID NO: 3051) | | | | | |
| TCR167 | EVDPIGHLY (SEQ | A0101 | 3058 | 3352 | 3663 | 3963 |
| | ID NO: 3051) | | | | | |
| TCR168 | EVDPIGHLY (SEQ | A0101 | 3103 | 3402 | 3710 | 4013 |
| | ID NO: 3051) | | | | | |
| TCR169 | EVDPIGHLY (SEQ | A0101 | 3104 | 3403 | 3711 | 4014 |
| | ID NO: 3051) | | | | | |
| TCR170 | EVDPIGHLY (SEQ | A0101 | 3105 | 3404 | 3712 | 4015 |
| | ID NO: 3051) | | | | | |
| TCR171 | EVDPIGHLY (SEQ | A0101 | 3106 | 3405 | 3713 | 4016 |
| | ID NO: 3051) | | | | | |
| TCR172 | EVDPIGHLY (SEQ | A0101 | 3107 | 3406 | 3714 | 4017 |
| | ID NO: 3051) | | | | | |
| TCR173 | EVDPIGHLY (SEQ | A0101 | 3108 | 3407 | 3715 | 4018 |
| | ID NO: 3051) | | | | | |
| TCR174 | EVDPIGHLY (SEQ | A0101 | 3109 | 3408 | 3716 | 4019 |
| | ID NO: 3051) | | | | | |
| TCR175 | EVDPIGHLY (SEQ | A0101 | 3110 | 3409 | 3717 | 4020 |
| | ID NO: 3051) | | | | | |
| TCR176 | EVDPIGHLY (SEQ | A0101 | 3111 | 3410 | 3718 | 4021 |
| TCR177 | EVDPIGHLY (SEQ | A0101 | 3112 | 3411 | 3719 | 4022 |
| | ID NO: 3051) | | | | | |
| TCR178 | EVDPIGHLY (SEQ | A0101 | 3113 | 3412 | 3720 | 4023 |
| | ID NO: 3051) | | | | | |
| TCR179 | EVDPIGHLY (SEQ | A0101 | 3058 | 3351 | 3663 | 3962 |
| | ID NO: 3051) | | | | | |
| TCR180 | EVDPIGHLY (SEQ | A0101 | 3052 | 3354 | 3659 | 3965 |
| | ID NO: 3051) | | | | | |
| TCR181 | EVDPIGHLY (SEQ | A0101 | 3072 | 3353 | 3677 | 4024 |
| | ID NO: 3051) | | | | | |
| TCR182 | EVDPIGHLY (SEQ | A0101 | 3052 | 3413 | 3721 | 4025 |
| | ID NO: 3051) | | | | | |
| TCR183 | EVDPIGHLY (SEQ | A0101 | 3114 | 3414 | 3722 | 4026 |
| | ID NO: 3051) | | | | | |
| TCR184 | EVDPIGHLY (SEQ | A0101 | 3058 | 3355 | 3663 | 3966 |
| | ID NO: 3051) | | | | | |
| TCR185 | EVDPIGHLY (SEQ | A0101 | 3052 | 3415 | 3659 | 4027 |
| | ID NO: 3051) | | | | | |
| TCR186 | EVDPIGHLY (SEQ | A0101 | 3114 | 3353 | 3722 | 3964 |
| | ID NO: 3051) | | | | | |
| TCR187 | EVDPIGHLY (SEQ | A0101 | 3115 | 3416 | 3723 | 4028 |
| | ID NO: 3051) | | | | | |
| TCR188 | EVDPIGHLY (SEQ | A0101 | 3116 | 3417 | 3724 | 4029 |
| | ID NO: 3051) | | | | | |
| TCR189 | EVDPIGHLY (SEQ | A0101 | 3117 | 3418 | 3725 | 4030 |
| | ID NO: 3051) | | | | | |
| TCR190 | EVDPIGHLY (SEQ | A0101 | 3118 | 3419 | 3726 | 4031 |
| | ID NO: 3051) | | | | | |
| TCR191 | EVDPIGHLY (SEQ | A0101 | 3119 | 3420 | 3727 | 4032 |
| | ID NO: 3051) | | | | | |
| TCR192 | EVDPIGHLY (SEQ | A0101 | 3120 | 3352 | 3728 | 4033 |
| | ID NO: 3051) | | | | | |
| TCR193 | EVDPIGHLY (SEQ | A0101 | 3121 | 3421 | 3729 | 4034 |
| | ID NO: 3051) | | | | | |
| TCR194 | EVDPIGHLY (SEQ | A0101 | 3054 | 3367 | 3658 | 3978 |
| | ID NO: 3051) | | | | | |
| TCR195 | EVDPIGHLY (SEQ | A0101 | 3122 | 3422 | 3730 | 4035 |
| | ID NO: 3051) | | | | | |
| TCR196 | EVDPIGHLY (SEQ | A0101 | 3123 | 3423 | 3731 | 4036 |
| | ID NO: 3051) | | | | | |
| TCR197 | EVDPIGHLY (SEQ | A0101 | 3124 | 3424 | 3732 | 4037 |
| | ID NO: 3051) | | | | | |
| TCR198 | EVDPIGHLY (SEQ | A0101 | 3112 | 3351 | 3719 | 3962 |
| | ID NO: 3051) | | | | | |
| TCR199 | EVDPIGHLY (SEQ | A0101 | 3060 | 3352 | 3665 | 3963 |
| | ID NO: 3051) | | | | | |
| TCR200 | EVDPIGHLY (SEQ | A0101 | 3059 | 3351 | 3664 | 3962 |
| | ID NO: 3051) | | | | | |
| TCR201 | EVDPIGHLY (SEQ | A0101 | 3071 | 3355 | 3676 | 3966 |
| | ID NO: 3051) | | | | | |
| TCR202 | EVDPIGHLY (SEQ | A0101 | 3125 | 3425 | 3733 | 4038 |
| | ID NO: 3051) | | | | | |
| TCR203 | EVDPIGHLY (SEQ | A0101 | 3126 | 3426 | *3734 | 4039 |
| | ID NO: 3051) | | | | | |
| TCR204 | EVDPIGHLY (SEQ | A0101 | 3127 | 3427 | 3735 | 4040 |
| | ID NO: 3051) | | | | | |
| TCR205 | EVDPIGHLY (SEQ | A0101 | 3128 | 3428 | 3736 | 4041 |
| | ID NO: 3051) | | | | | |
| TCR206 | EVDPIGHLY (SEQ | A0101 | 3129 | 3429 | 3737 | 4042 |
| | ID NO: 3051) | | | | | |
| TCR207 | EVDPIGHLY (SEQ | A0101 | 3130 | 3352 | 3738 | 3963 |
| | ID NO: 3051) | | | | | |
| TCR208 | EVDPIGHLY (SEQ | A0101 | 3052 | 3362 | 3659 | 3973 |
| | ID NO: 3051) | | | | | |
| TCR209 | EVDPIGHLY (SEQ | A0101 | 3055 | 3352 | 3660 | 3963 |
| | ID NO: 3051) | | | | | |
| TCR210 | EVDPIGHLY (SEQ | A0101 | 3131 | 3430 | 3739 | 4043 |
| | ID NO: 3051) | | | | | |
| TCR211 | EVDPIGHLY (SEQ | A0101 | 3132 | 3431 | 3740 | 4044 |
| | ID NO: 3051) | | | | | |
| TCR212 | EVDPIGHLY (SEQ | A0101 | 3133 | 3432 | 3741 | 4045 |
| | ID NO: 3051) | | | | | |
| TCR213 | EVDPIGHLY (SEQ | A0101 | 3053 | 3381 | 3657 | 3992 |
| | ID NO: 3051) | | | | | |
| TCR214 | EVDPIGHLY (SEQ | A0101 | 3134 | 3433 | 3742 | 4046 |
| | ID NO: 3051) | | | | | |
| TCR215 | EVDPIGHLY (SEQ | A0101 | 3061 | 3351 | 3666 | 3962 |
| | ID NO: 3051) | | | | | |
| TCR216 | EVDPIGHLY (SEQ | A0101 | 3104 | 3352 | 3711 | 3963 |
| | ID NO: 3051) | | | | | |
| TCR217 | EVDPIGHLY (SEQ | A0101 | 3055 | 3351 | 3660 | 3962 |
| | ID NO: 3051) | | | | | |
| TCR218 | EVDPIGHLY (SEQ | A0101 | 3058 | 3353 | 3663 | 3964 |
| | ID NO: 3051) | | | | | |
| TCR219 | EVDPIGHLY (SEQ | A0101 | 3135 | 3434 | 3743 | 4047 |
| | ID NO: 3051) | | | | | |
| TCR220 | EVDPIGHLY (SEQ | A0101 | 3052 | 3435 | 3744 | 4048 |
| | ID NO: 3051) | | | | | |
| TCR221 | EVDPIGHLY (SEQ | A0101 | 3136 | 3436 | 3745 | 4049 |
| | ID NO: 3051) | | | | | |
| TCR222 | EVDPIGHLY (SEQ | A0101 | 3137 | 3437 | 3746 | 4050 |
| | ID NO: 3051) | | | | | |
| TCR223 | EVDPIGHLY (SEQ | A0101 | 3138 | 3438 | 3747 | 4051 |
| | ID NO: 3051) | | | | | |
| TCR224 | EVDPIGHLY (SEQ | A0101 | 3139 | 3439 | 3748 | 4052 |
| | ID NO: 3051) | | | | | |
| TCR225 | EVDPIGHLY (SEQ | A0101 | 3140 | 3440 | 3749 | 4053 |
| | ID NO: 3051) | | | | | |
| TCR226 | EVDPIGHLY (SEQ | A0101 | 3141 | 3441 | 3750 | 4054 |
| | ID NO: 3051) | | | | | |
| TCR227 | EVDPIGHLY (SEQ | A0101 | 3142 | 3442 | 3751 | 4055 |
| | ID NO: 3051) | | | | | |
| TCR228 | EVDPIGHLY (SEQ | A0101 | 3143 | 3443 | 3752 | 4056 |
| | ID NO: 3051) | | | | | |
| TCR229 | EVDPIGHLY (SEQ | A0101 | 3144 | 3444 | 3753 | 4057 |
| | ID NO: 3051) | | | | | |
| TCR230 | EVDPIGHLY (SEQ | A0101 | 3145 | 3445 | 3754 | 4058 |
| | ID NO: 3051) | | | | | |
| TCR231 | EVDPIGHLY (SEQ | A0101 | 3136 | 3444 | 3755 | 4057 |
| | ID NO: 3051) | | | | | |
| TCR232 | EVDPIGHLY (SEQ | A0101 | 3146 | 3446 | 3756 | 4059 |
| | ID NO: 3051) | | | | | |
| TCR233 | EVDPIGHLY (SEQ | A0101 | 3147 | 3447 | 3757 | 4060 |
| | ID NO: 3051) | | | | | |
| TCR234 | EVDPIGHLY (SEQ | A0101 | 3148 | 3448 | 3758 | 4061 |
| | ID NO: 3051) | | | | | |
| TCR235 | EVDPIGHLY (SEQ | A0101 | 3149 | 3449 | 3759 | 4062 |
| | ID NO: 3051) | | | | | |
| TCR236 | EVDPIGHLY (SEQ | A0101 | 3150 | 3450 | 3760 | 4063 |
| | ID NO: 3051) | | | | | |
| TCR237 | EVDPIGHLY (SEQ | A0101 | 3151 | 3436 | 3761 | 4049 |
| | ID NO: 3051) | | | | | |
| TCR238 | EVDPIGHLY (SEQ | A0101 | 3139 | 3436 | 3748 | 4049 |
| | ID NO: 3051) | | | | | |
| TCR239 | EVDPIGHLY (SEQ | A0101 | 3152 | 3451 | 3762 | 4064 |
| | ID NO: 3051) | | | | | |
| TCR240 | EVDPIGHLY (SEQ | A0101 | 3153 | 3452 | 3763 | 4065 |
| | ID NO: 3051) | | | | | |
| TCR241 | EVDPIGHLY (SEQ | A0101 | 3154 | 3453 | 3764 | 4066 |
| | ID NO: 3051) | | | | | |
| TCR242 | EVDPIGHLY (SEQ | A0101 | 3155 | 3454 | 3765 | 4067 |
| | ID NO: 3051) | | | | | |
| TCR243 | EVDPIGHLY (SEQ | A0101 | 3137 | 3440 | 3746 | 4053 |
| TCR244 | EVDPIGHLY (SEQ | A0101 | 3156 | 3455 | 3766 | 4068 |
| | ID NO: 3051) | | | | | |
| TCR245 | EVDPIGHLY (SEQ | A0101 | 3151 | 3456 | 3761 | 4069 |
| | ID NO: 3051) | | | | | |
| TCR246 | EVDPIGHLY (SEQ | A0101 | 3157 | 3457 | 3767 | 4070 |
| | ID NO: 3051) | | | | | |
| TCR247 | EVDPIGHLY (SEQ | A0101 | 3158 | 3458 | 3768 | 4071 |
| | ID NO: 3051) | | | | | |
| TCR248 | EVDPIGHLY (SEQ | A0101 | 3159 | 3459 | 3769 | 4072 |
| | ID NO: 3051) | | | | | |
| TCR249 | EVDPIGHLY (SEQ | A0101 | 3160 | 3460 | 3770 | 4073 |
| | ID NO: 3051) | | | | | |
| TCR250 | EVDPIGHLY (SEQ | A0101 | 3077 | 3461 | 3771 | 4074 |
| | ID NO: 3051) | | | | | |
| TCR251 | EVDPIGHLY (SEQ | A0101 | 3161 | 3462 | 3772 | 4075 |
| | ID NO: 3051) | | | | | |
| TCR252 | EVDPIGHLY (SEQ | A0101 | 3162 | 3463 | 3773 | 4076 |
| | ID NO: 3051) | | | | | |
| TCR253 | EVDPIGHLY (SEQ | A0101 | 3163 | 3464 | 3774 | 4077 |
| | ID NO: 3051) | | | | | |
| TCR254 | EVDPIGHLY (SEQ | A0101 | 3164 | 3465 | 3775 | 4078 |
| | ID NO: 3051) | | | | | |
| TCR255 | EVDPIGHLY (SEQ | A0101 | 3137 | 3442 | 3746 | 4055 |
| | ID NO: 3051) | | | | | |
| TCR256 | EVDPIGHLY (SEQ | A0101 | 3136 | 3438 | 3745 | 4051 |
| | ID NO: 3051) | | | | | |
| TCR257 | EVDPIGHLY (SEQ | A0101 | 3165 | 3466 | 3776 | 4079 |
| | ID NO: 3051) | | | | | |
| TCR258 | EVDPIGHLY (SEQ | A0101 | 3166 | 3467 | 3777 | 4080 |
| | ID NO: 3051) | | | | | |
| TCR259 | EVDPIGHLY (SEQ | A0101 | 3167 | 3468 | 3778 | 4081 |
| | ID NO: 3051) | | | | | |
| TCR260 | EVDPIGHLY (SEQ | A0101 | 3168 | 3469 | 3779 | 4082 |
| | ID NO: 3051) | | | | | |
| TCR261 | EVDPIGHLY (SEQ | A0101 | 3169 | 3470 | 3780 | 4083 |
| | ID NO: 3051) | | | | | |
| TCR262 | EVDPIGHLY (SEQ | A0101 | 3137 | 3436 | 3746 | 4049 |
| | ID NO: 3051) | | | | | |
| TCR263 | EVDPIGHLY (SEQ | A0101 | 3170 | 3471 | 3781 | 4084 |
| | ID NO: 3051) | | | | | |
| TCR264 | EVDPIGHLY (SEQ | A0101 | 3171 | 3472 | 3782 | 4085 |
| | ID NO: 3051) | | | | | |
| TCR265 | EVDPIGHLY (SEQ | A0101 | 3172 | 3473 | 3783 | 4086 |
| | ID NO: 3051) | | | | | |
| TCR266 | EVDPIGHLY (SEQ | A0101 | 3173 | 3474 | 3784 | 4087 |
| | ID NO: 3051) | | | | | |
| TCR267 | EVDPIGHLY (SEQ | A0101 | 3174 | 3475 | 3785 | 4088 |
| | ID NO: 3051) | | | | | |
| TCR268 | EVDPIGHLY (SEQ | A0101 | 3175 | 3476 | 3786 | 4089 |
| | ID NO: 3051) | | | | | |
| TCR269 | EVDPIGHLY (SEQ | A0101 | 3176 | 3477 | 3787 | 4090 |
| | ID NO: 3051) | | | | | |
| TCR270 | EVDPIGHLY (SEQ | A0101 | 3177 | 3478 | 3788 | 4091 |
| | ID NO: 3051) | | | | | |
| TCR271 | EVDPIGHLY (SEQ | A0101 | 3178 | 3479 | 3789 | 4092 |
| | ID NO: 3051) | | | | | |
| TCR272 | EVDPIGHLY (SEQ | A0101 | 3179 | 3480 | 3790 | 4093 |
| | ID NO: 3051) | | | | | |
| TCR273 | EVDPIGHLY (SEQ | A0101 | 3180 | 3481 | 3791 | 4094 |
| | ID NO: 3051) | | | | | |
| TCR274 | EVDPIGHLY (SEQ | A0101 | 3136 | 3482 | 3755 | 4095 |
| | ID NO: 3051) | | | | | |
| TCR275 | EVDPIGHLY (SEQ | A0101 | 3181 | 3483 | 3792 | 4096 |
| | ID NO: 3051) | | | | | |
| TCR276 | EVDPIGHLY (SEQ | A0101 | 3182 | 3484 | 3793 | 4097 |
| | ID NO: 3051) | | | | | |
| TCR277 | EVDPIGHLY (SEQ | A0101 | 3183 | 3485 | 3794 | 4098 |
| | ID NO: 3051) | | | | | |
| TCR278 | EVDPIGHLY (SEQ | A0101 | 3184 | 3486 | 3795 | 4099 |
| | ID NO: 3051) | | | | | |
| TCR279 | EVDPIGHLY (SEQ | A0101 | 3185 | 3487 | 3796 | 4100 |
| | ID NO: 3051) | | | | | |
| TCR280 | EVDPIGHLY (SEQ | A0101 | 3186 | 3488 | 3797 | 4101 |
| | ID NO: 3051) | | | | | |
| TCR281 | EVDPIGHLY (SEQ | A0101 | 3187 | 3489 | 3798 | 4102 |
| | ID NO: 3051) | | | | | |
| TCR282 | EVDPIGHLY (SEQ | A0101 | 3188 | 3482 | 3799 | 4095 |
| | ID NO: 3051) | | | | | |
| TCR283 | EVDPIGHLY (SEQ | A0101 | 3189 | 3490 | 3800 | 4103 |
| | ID NO: 3051) | | | | | |
| TCR284 | EVDPIGHLY (SEQ | A0101 | 3190 | 3491 | 3801 | 4104 |
| | ID NO: 3051) | | | | | |
| TCR285 | EVDPIGHLY (SEQ | A0101 | 3191 | 3492 | 3802 | 4105 |
| | ID NO: 3051) | | | | | |
| TCR286 | EVDPIGHLY (SEQ | A0101 | 3192 | 3493 | 3803 | 4106 |
| | ID NO: 3051) | | | | | |
| TCR287 | EVDPIGHLY (SEQ | A0101 | 3193 | 3494 | 3804 | 4107 |
| | ID NO: 3051) | | | | | |
| TCR288 | EVDPIGHLY (SEQ | A0101 | 3194 | 3495 | 3805 | 4108 |
| | ID NO: 3051) | | | | | |
| TCR289 | EVDPIGHLY (SEQ | A0101 | 3195 | 3496 | 3806 | 4109 |
| | ID NO: 3051) | | | | | |
| TCR290 | EVDPIGHLY (SEQ | A0101 | 3196 | 3497 | 3807 | 4110 |
| | ID NO: 3051) | | | | | |
| TCR291 | EVDPIGHLY (SEQ | A0101 | 3197 | 3498 | 3808 | 4111 |
| | ID NO: 3051) | | | | | |
| TCR292 | EVDPIGHLY (SEQ | A0101 | 3198 | 3499 | 3809 | 4112 |
| | ID NO: 3051) | | | | | |
| TCR293 | EVDPIGHLY (SEQ | A0101 | 3199 | 3500 | 3810 | 4113 |
| | ID NO: 3051) | | | | | |
| TCR294 | EVDPIGHLY (SEQ | A0101 | 3137 | 3449 | 3746 | 4062 |
| | ID NO: 3051) | | | | | |
| TCR295 | EVDPIGHLY (SEQ | A0101 | 3200 | 3436 | 3811 | 4049 |
| | ID NO: 3051) | | | | | |
| TCR296 | EVDPIGHLY (SEQ | A0101 | 3201 | 3501 | 3812 | 4114 |
| | ID NO: 3051) | | | | | |
| TCR297 | EVDPIGHLY (SEQ | A0101 | 3138 | 3436 | 3747 | 4049 |
| | ID NO: 3051) | | | | | |
| TCR298 | EVDPIGHLY (SEQ | A0101 | 3202 | 3502 | 3813 | 4115 |
| | ID NO: 3051) | | | | | |
| TCR299 | EVDPIGHLY (SEQ | A0101 | 3203 | 3503 | 3814 | 4116 |
| | ID NO: 3051) | | | | | |
| TCR300 | EVDPIGHLY (SEQ | A0101 | 3204 | 3504 | 3815 | 4117 |
| | ID NO: 3051) | | | | | |
| TCR301 | EVDPIGHLY (SEQ | A0101 | 3205 | 3505 | 3816 | 4118 |
| | ID NO: 3051) | | | | | |
| TCR302 | EVDPIGHLY (SEQ | A0101 | 3206 | 3506 | 3817 | 4119 |
| | ID NO: 3051) | | | | | |
| TCR303 | EVDPIGHLY (SEQ | A0101 | 3207 | 3507 | 3818 | 4120 |
| | ID NO: 3051) | | | | | |
| TCR304 | EVDPIGHLY (SEQ | A0101 | 3148 | 3440 | 3758 | 4053 |
| | ID NO: 3051) | | | | | |
| TCR305 | EVDPIGHLY (SEQ | A0101 | 3208 | 3508 | 3819 | 4121 |
| | ID NO: 3051) | | | | | |
| TCR306 | EVDPIGHLY (SEQ | A0101 | 3209 | 3509 | 3820 | 4122 |
| | ID NO: 3051) | | | | | |
| TCR307 | EVDPIGHLY (SEQ | A0101 | 3210 | 3510 | 3821 | 4123 |
| | ID NO: 3051) | | | | | |
| TCR308 | EVDPIGHLY (SEQ | A0101 | 3211 | 3511 | 3822 | 4124 |
| | ID NO: 3051) | | | | | |
| TCR309 | EVDPIGHLY (SEQ | A0101 | 3212 | 3512 | 3823 | 4125 |
| | ID NO: 3051) | | | | | |
| TCR310 | EVDPIGHLY (SEQ | A0101 | 3213 | 3513 | 3824 | 4126 |
| TCR311 | EVDPIGHLY (SEQ | A0101 | 3214 | 3514 | 3825 | 4127 |
| | ID NO: 3051) | | | | | |
| TCR312 | EVDPIGHLY (SEQ | A0101 | 3215 | 3515 | 3826 | 4128 |
| | ID NO: 3051) | | | | | |
| TCR313 | EVDPIGHLY (SEQ | A0101 | 3216 | 3516 | 3827 | 4129 |
| | ID NO: 3051) | | | | | |
| TCR314 | EVDPIGHLY (SEQ | A0101 | 3217 | 3517 | 3828 | 4130 |
| | ID NO: 3051) | | | | | |
| TCR315 | EVDPIGHLY (SEQ | A0101 | 3218 | 3518 | 3829 | 4131 |
| | ID NO: 3051) | | | | | |
| TCR316 | EVDPIGHLY (SEQ | A0101 | 3219 | 3519 | 3830 | 4132 |
| | ID NO: 3051) | | | | | |
| TCR3I7 | EVDPIGHLY (SEQ | A0101 | 3220 | 3520 | 3831 | 4133 |
| | ID NO: 3051) | | | | | |
| TCR318 | EVDPIGHLY (SEQ | A0101 | 3221 | 3521 | 3832 | 4134 |
| | ID NO: 3051) | | | | | |
| TCR319 | EVDPIGHLY (SEQ | A0101 | 3217 | 3518 | 3828 | 4131 |
| | ID NO: 3051) | | | | | |
| TCR320 | EVDPIGHLY (SEQ | A0101 | 3222 | 3522 | 3833 | 4135 |
| | ID NO: 3051) | | | | | |
| TCR321 | EVDPIGHLY (SEQ | A0101 | 3223 | 3523 | 3834 | 4136 |
| | ID NO: 3051) | | | | | |
| TCR322 | EVDPIGHLY (SEQ | A0101 | 3224 | 3524 | 3835 | 4137 |
| | ID NO: 3051) | | | | | |
| TCR323 | EVDPIGHLY (SEQ | A0101 | 3225 | 3525 | 3836 | 4138 |
| | ID NO: 3051) | | | | | |
| TCR324 | EVDPIGHLY (SEQ | A0101 | 3226 | 3526 | 3837 | 4139 |
| | ID NO: 3051) | | | | | |
| TCR325 | EVDPIGHLY (SEQ | A0101 | 3227 | 3527 | 3838 | 4140 |
| | ID NO: 3051) | | | | | |
| TCR326 | EVDPIGHLY (SEQ | A0101 | 3228 | 3528 | 3839 | 4141 |
| | ID NO: 3051) | | | | | |
| TCR327 | EVDPIGHLY (SEQ | A0101 | 3229 | 3529 | 3840 | 4142 |
| | ID NO: 3051) | | | | | |
| TCR328 | EVDPIGHLY (SEQ | A0101 | 3230 | 3530 | 3841 | 4143 |
| | ID NO: 3051) | | | | | |
| TCR329 | EVDPIGHLY (SEQ | A0101 | 3217 | 3525 | 3828 | 4138 |
| | ID NO: 3051) | | | | | |
| TCR330 | EVDPIGHLY (SEQ | A0101 | 3231 | 3531 | 3842 | 4144 |
| | ID NO: 3051) | | | | | |
| TCR331 | EVDPIGHLY (SEQ | A0101 | 3232 | 3532 | 3843 | 4145 |
| | ID NO: 3051) | | | | | |
| TCR332 | EVDPIGHLY (SEQ | A0101 | 3233 | 3520 | 3844 | 4133 |
| | ID NO: 3051) | | | | | |
| TCR333 | EVDPIGHLY (SEQ | A0101 | 3217 | 3530 | 3828 | 4143 |
| | ID NO: 3051) | | | | | |
| TCR334 | EVDPIGHLY (SEQ | A0101 | 3234 | 3533 | 3845 | 4146 |
| | ID NO: 3051) | | | | | |
| TCR335 | EVDPIGHLY (SEQ | A0101 | 3235 | 3534 | 3846 | 4147 |
| | ID NO: 3051) | | | | | |
| TCR336 | EVDPIGHLY (SEQ | A0101 | 3217 | 3532 | 3828 | 4145 |
| | ID NO: 3051) | | | | | |
| TCR337 | EVDPIGHLY (SEQ | A0101 | 3236 | 3535 | 3847 | 4148 |
| | ID NO: 3051) | | | | | |
| TCR338 | EVDPIGHLY (SEQ | A0101 | 3237 | 3536 | 3848 | 4149 |
| | ID NO: 3051) | | | | | |
| TCR339 | EVDPIGHLY (SEQ | A0101 | 3238 | 3537 | 3849 | 4150 |
| | ID NO: 3051) | | | | | |
| TCR340 | EVDPIGHLY (SEQ | A0101 | 3239 | 3538 | 3850 | 4151 |
| | ID NO: 3051) | | | | | |
| TCR341 | EVDPIGHLY (SEQ | A0101 | 3240 | 3539 | 3851 | 4152 |
| | ID NO: 3051) | | | | | |
| TCR342 | EVDPIGHLY (SEQ | A0101 | 3241 | 3540 | 3852 | 4153 |
| | ID NO: 3051) | | | | | |
| TCR343 | EVDPIGHLY (SEQ | A0101 | 3242 | 3541 | 3853 | 4154 |
| | ID NO: 3051) | | | | | |
| TCR344 | EVDPIGHLY (SEQ | A0101 | 3243 | 3542 | 3854 | 4155 |
| | ID NO: 3051) | | | | | |
| TCR345 | EVDPIGHLY (SEQ | A0101 | 3244 | 3543 | 3855 | 4156 |
| | ID NO: 3051) | | | | | |
| TCR346 | EVDPIGHLY (SEQ | A0101 | 3245 | 3544 | 3831 | 4157 |
| | ID NO: 3051) | | | | | |
| TCR347 | EVDPIGHLY (SEQ | A0101 | 3246 | 3545 | 3856 | 4158 |
| | ID NO: 3051) | | | | | |
| TCR348 | EVDPIGHLY (SEQ | A0101 | 3247 | 3546 | 3857 | 4159 |
| | ID NO: 3051) | | | | | |
| TCR349 | EVDPIGHLY (SEQ | A0101 | 3248 | 3547 | 3858 | 4160 |
| | ID NO: 3051) | | | | | |
| TCR350 | EVDPIGHLY (SEQ | A0101 | 3249 | 3548 | 3859 | 4161 |
| | ID NO: 3051) | | | | | |
| TCR351 | EVDPIGHLY (SEQ | A0101 | 3217 | 3524 | 3828 | 4137 |
| | ID NO: 3051) | | | | | |
| TCR352 | EVDPIGHLY (SEQ | A0101 | 3250 | 3549 | 3860 | 4162 |
| | ID NO: 3051) | | | | | |
| TCR353 | EVDPIGHLY (SEQ | A0101 | 3251 | 3550 | 3861 | 4163 |
| | ID NO: 3051) | | | | | |
| TCR354 | EVDPIGHLY (SEQ | A0101 | 3252 | 3551 | 3862 | 4164 |
| | ID NO: 3051) | | | | | |
| TCR355 | EVDPIGHLY (SEQ | A0101 | 3253 | 3552 | 3863 | 4165 |
| | ID NO: 3051) | | | | | |
| TCR356 | EVDPIGHLY (SEQ | A0101 | 3254 | 3553 | 3864 | 4166 |
| | ID NO: 3051) | | | | | |
| TCR357 | EVDPIGHLY (SEQ | A0101 | 3255 | 3554 | 3865 | 4167 |
| | ID NO: 3051) | | | | | |
| TCR358 | EVDPIGHLY (SEQ | A0101 | 3256 | 3555 | 3866 | 4168 |
| | ID NO: 3051) | | | | | |
| TCR359 | EVDPIGHLY (SEQ | A0101 | 3257 | 3556 | 3867 | 4169 |
| | ID NO: 3051) | | | | | |
| TCR360 | EVDPIGHLY (SEQ | A0101 | 3258 | 3557 | 3868 | 4170 |
| | ID NO: 3051) | | | | | |
| TCR361 | EVDPIGHLY (SEQ | A0101 | 3259 | 3558 | 3869 | 4171 |
| | ID NO: 3051) | | | | | |
| TCR362 | EVDPIGHLY (SEQ | A0101 | 3260 | 3559 | 3870 | 4172 |
| | ID NO: 3051) | | | | | |
| TCR363 | EVDPIGHLY (SEQ | A0101 | 3261 | 3560 | 3871 | 4173 |
| | ID NO: 3051) | | | | | |
| TCR364 | EVDPIGHLY (SEQ | A0101 | 3217 | 3519 | 3828 | 4132 |
| | ID NO: 3051) | | | | | |
| TCR365 | EVDPIGHLY (SEQ | A0101 | 3262 | 3561 | 3872 | 4174 |
| | ID NO: 3051) | | | | | |
| TCR366 | EVDPIGHLY (SEQ | A0101 | 3263 | 3562 | 3873 | 4175 |
| | ID NO: 3051) | | | | | |
| TCR367 | EVDPIGHLY (SEQ | A0101 | 3217 | 3563 | 3828 | 4176 |
| | ID NO: 3051) | | | | | |
| TCR368 | EVDPIGHLY (SEQ | A0101 | 3264 | 3564 | 3874 | 4177 |
| | ID NO: 3051) | | | | | |
| TCR369 | EVDPIGHLY (SEQ | A0101 | 3265 | 3565 | 3875 | 4178 |
| | ID NO: 3051) | | | | | |
| TCR370 | EVDPIGHLY (SEQ | A0101 | 3266 | 3566 | 3876 | 4179 |
| | ID NO: 3051) | | | | | |
| TCR371 | EVDPIGHLY (SEQ | A0101 | 3267 | 3567 | 3877 | 4180 |
| | ID NO: 3051) | | | | | |
| TCR372 | EVDPIGHLY (SEQ | A0101 | 3268 | 3568 | 3878 | 4181 |
| | ID NO: 3051) | | | | | |
| TCR373 | EVDPIGHLY (SEQ | A0101 | 3269 | 3569 | 3879 | 4182 |
| | ID NO: 3051) | | | | | |
| TCR374 | EVDPIGHLY (SEQ | A0101 | 3217 | 3528 | 3828 | 4141 |
| | ID NO: 3051) | | | | | |
| TCR375 | EVDPIGHLY (SEQ | A0101 | 3270 | 3570 | 3880 | 4183 |
| | ID NO: 3051) | | | | | |
| TCR376 | EVDPIGHLY (SEQ | A0101 | 3217 | 3571 | 3828 | 4184 |
| | ID NO: 3051) | | | | | |
| TCR377 | EVDPIGHLY (SEQ | A0101 | 3271 | 3572 | 3881 | 4185 |
| TCR378 | EVDPIGHLY (SEQ | A0101 | 3219 | 3522 | 3830 | 4135 |
| | ID NO: 3051) | | | | | |
| TCR379 | EVDPIGHLY (SEQ | A0101 | 3272 | 3573 | 3882 | 4186 |
| | ID NO: 3051) | | | | | |
| TCR380 | EVDPIGHLY (SEQ | A0101 | 3273 | 3574 | 3883 | 4187 |
| | ID NO: 3051) | | | | | |
| TCR381 | EVDPIGHLY (SEQ | A0101 | 3274 | 3575 | 3884 | 4188 |
| | ID NO: 3051) | | | | | |
| TCR382 | EVDPIGHLY (SEQ | A0101 | 3275 | 3576 | 3885 | 4189 |
| | ID NO: 3051) | | | | | |
| TCR383 | EVDPIGHLY (SEQ | A0101 | 3217 | 3577 | 3828 | 4190 |
| | ID NO: 3051) | | | | | |
| TCR384 | EVDPIGHLY (SEQ | A0101 | 3230 | 3517 | 3841 | 4130 |
| | ID NO: 3051) | | | | | |
| TCR385 | EVDPIGHLY (SEQ | A0101 | 3276 | 3578 | 3886 | 4191 |
| | ID NO: 3051) | | | | | |
| TCR386 | EVDPIGHLY (SEQ | A0101 | 3277 | 3579 | 3887 | 4192 |
| | ID NO: 3051) | | | | | |
| TCR387 | EVDPIGHLY (SEQ | A0101 | 3278 | 3580 | 3888 | 4193 |
| | ID NO: 3051) | | | | | |
| TCR388 | EVDPIGHLY (SEQ | A0101 | 3279 | 3581 | 3889 | 4194 |
| v | | | | | | |
| TCR389 | EVDPIGHLY (SEQ | A0101 | 3280 | 3582 | 3890 | 4195 |
| | ID NO: 3051) | | | | | |
| TCR390 | EVDPIGHLY (SEQ | A0101 | 3281 | 3583 | 3891 | 4196 |
| | ID NO: 3051) | | | | | |
| TCR391 | EVDPIGHLY (SEQ | A0101 | 3282 | 3584 | 3892 | 4197 |
| | ID NO: 3051) | | | | | |
| TCR392 | EVDPIGHLY (SEQ | A0101 | 3283 | 3585 | 3893 | 4198 |
| | ID NO: 3051) | | | | | |
| TCR393 | EVDPIGHLY (SEQ | A0101 | 3284 | 3586 | 3894 | 4199 |
| | ID NO: 3051) | | | | | |
| TCR394 | EVDPIGHLY (SEQ | A0101 | 3285 | 3587 | 3895 | 4200 |
| | ID NO: 3051) | | | | | |
| TCR395 | EVDPIGHLY (SEQ | A0101 | 3286 | 3588 | 3896 | 4201 |
| | ID NO: 3051) | | | | | |
| TCR396 | EVDPIGHLY (SEQ | A0101 | 3287 | 3589 | 3897 | 4202 |
| | ID NO: 3051) | | | | | |
| TCR397 | EVDPIGHLY (SEQ | A0101 | 3288 | 3590 | 3898 | 4203 |
| | ID NO: 3051) | | | | | |
| TCR398 | EVDPIGHLY (SEQ | A0101 | 3289 | 3591 | 3899 | 4204 |
| | ID NO: 3051) | | | | | |
| TCR399 | EVDPIGHLY (SEQ | A0101 | 3290 | 3592 | 3900 | 4205 |
| | ID NO: 3051) | | | | | |
| TCR400 | EVDPIGHLY (SEQ | A0101 | 3291 | 3593 | 3901 | 4206 |
| | ID NO: 3051) | | | | | |
| TCR401 | EVDPIGHLY (SEQ | A0101 | 3292 | 3594 | 3902 | 4207 |
| | ID NO: 3051) | | | | | |
| TCR402 | EVDPIGHLY (SEQ | A0101 | 3293 | 3595 | 3903 | 4208 |
| | ID NO: 3051) | | | | | |
| TCR403 | EVDPIGHLY (SEQ | A0101 | 3294 | 3596 | 3904 | 4209 |
| | ID NO: 3051) | | | | | |
| TCR404 | EVDPIGHLY (SEQ | A0101 | 3295 | 3597 | 3905 | 4210 |
| | ID NO: 3051) | | | | | |
| TCR405 | EVDPIGHLY (SEQ | A0101 | 3219 | 3598 | 3830 | 4211 |
| | ID NO: 3051) | | | | | |
| TCR406 | EVDPIGHLY (SEQ | A0101 | 3296 | 3599 | 3906 | 4212 |
| | ID NO: 3051) | | | | | |
| TCR407 | EVDPIGHLY (SEQ | A0101 | 3217 | 3600 | 3828 | 4213 |
| | ID NO: 3051) | | | | | |
| TCR408 | EVDPIGHLY (SEQ | A0101 | 3297 | 3601 | 3907 | 4214 |
| | ID NO: 3051) | | | | | |
| TCR409 | EVDPIGHLY (SEQ | A0101 | 3298 | 3602 | 3908 | 4215 |
| | ID NO: 3051) | | | | | |
| TCR410 | EVDPIGHLY (SEQ | A0101 | 3299 | 3603 | 3909 | 4216 |
| | ID NO: 3051) | | | | | |
| TCR411 | EVDPIGHLY (SEQ | A0101 | 3300 | 3604 | 3910 | 4217 |
| | ID NO: 3051) | | | | | |
| TCR412 | EVDPIGHLY (SEQ | A0101 | 3301 | 3605 | 3911 | 4218 |
| | ID NO: 3051) | | | | | |
| TCR413 | EVDPIGHLY (SEQ | A0101 | 3302 | 3606 | 3912 | 4219 |
| | ID NO: 3051) | | | | | |
| TCR414 | EVDPIGHLY (SEQ | A0101 | 3303 | 3607 | 3913 | 4220 |
| | ID NO: 3051) | | | | | |
| TCR415 | EVDPIGHLY (SEQ | A0101 | 3304 | 3608 | 3914 | 4221 |
| | ID NO: 3051) | | | | | |
| TCR416 | EVDPIGHLY (SEQ | A0101 | 3305 | 3609 | 3915 | 4222 |
| | ID NO: 3051) | | | | | |
| TCR417 | EVDPIGHLY (SEQ | A0101 | 3306 | 3610 | 3916 | 4223 |
| | ID NO: 3051) | | | | | |
| TCR418 | EVDPIGHLY (SEQ | A0101 | 3307 | 3611 | 3917 | 4224 |
| | ID NO: 3051) | | | | | |
| TCR419 | EVDPIGHLY (SEQ | A0101 | 3289 | 3595 | 3899 | 4208 |
| | ID NO: 3051) | | | | | |
| TCR420 | EVDPIGHLY (SEQ | A0101 | 3308 | 3612 | 3918 | 4225 |
| | ID NO: 3051) | | | | | |
| TCR421 | EVDPIGHLY (SEQ | A0101 | 3309 | 3613 | 3919 | 4226 |
| | ID NO: 3051) | | | | | |
| TCR422 | EVDPIGHLY (SEQ | A0101 | 3310 | 3614 | 3920 | 4227 |
| | ID NO: 3051) | | | | | |
| TCR423 | EVDPIGHLY (SEQ | A0101 | 3311 | 3615 | 3921 | 4228 |
| | ID NO: 3051) | | | | | |
| TCR424 | EVDPIGHLY (SEQ | A0101 | 3312 | 3616 | 3922 | 4229 |
| | ID NO: 3051) | | | | | |
| TCR425 | EVDPIGHLY (SEQ | A0101 | 3313 | 3617 | 3923 | 4230 |
| | ID NO: 3051) | | | | | |
| TCR426 | EVDPIGHLY (SEQ | A0101 | 3314 | 3618 | 3924 | 4231 |
| | ID NO: 3051) | | | | | |
| TCR427 | EVDPIGHLY (SEQ | A0101 | 3289 | 3619 | 3899 | 4232 |
| | ID NO: 3051) | | | | | |
| TCR428 | EVDPIGHLY (SEQ | A0101 | 3315 | 3620 | 3925 | 4233 |
| | ID NO: 3051) | | | | | |
| TCR429 | EVDPIGHLY (SEQ | A0101 | 3316 | 3621 | 3926 | 4234 |
| v | | | | | | |
| TCR430 | EVDPIGHLY (SEQ | A0101 | 3317 | 3622 | 3927 | 4235 |
| | ID NO: 3051) | | | | | |
| TCR431 | EVDPIGHLY (SEQ | A0101 | 3318 | 3623 | 3928 | 4236 |
| | ID NO: 3051) | | | | | |
| TCR432 | EVDPIGHLY (SEQ | A0101 | 3319 | 3624 | 3929 | 4237 |
| | ID NO: 3051) | | | | | |
| TCR433 | EVDPIGHLY (SEQ | A0101 | 3320 | 3625 | 3930 | 4238 |
| | ID NO: 3051) | | | | | |
| TCR434 | EVDPIGHLY (SEQ | A0101 | 3321 | 3626 | 3931 | 4239 |
| | ID NO: 3051) | | | | | |
| TCR435 | EVDPIGHLY (SEQ | A0101 | 3322 | 3627 | 3932 | 4240 |
| | ID NO: 3051) | | | | | |
| TCR436 | EVDPIGHLY (SEQ | A0101 | 3323 | 3628 | 3933 | 4241 |
| | ID NO: 3051) | | | | | |
| TCR437 | EVDPIGHLY (SEQ | A0101 | 3324 | 3629 | 3934 | 4242 |
| | ID NO: 3051) | | | | | |
| TCR438 | EVDPIGHLY (SEQ | A0101 | 3325 | 3602 | 3935 | 4215 |
| | ID NO: 3051) | | | | | |
| TCR439 | EVDPIGHLY (SEQ | A0101 | 3326 | 3630 | 3936 | 4243 |
| | ID NO: 3051) | | | | | |
| TCR440 | EVDPIGHLY (SEQ | A0101 | 3327 | 3631 | 3937 | 4244 |
| | ID NO: 3051) | | | | | |
| TCR441 | EVDPIGHLY (SEQ | A0101 | 3328 | 3632 | 3938 | 4245 |
| | ID NO: 3051) | | | | | |
| TCR442 | EVDPIGHLY (SEQ | A0101 | 3289 | 3598 | 3899 | 4211 |
| | ID NO: 3051) | | | | | |
| TCR443 | EVDPIGHLY (SEQ | A0101 | 3329 | 3633 | 3939 | 4246 |
| | ID NO: 3051) | | | | | |
| TCR444 | EVDPIGHLY (SEQ | A0101 | 3330 | 3634 | 3940 | 4247 |
| TCR445 | EVDPIGHLY (SEQ | A0101 | 3331 | 3635 | 3941 | 4248 |
| | ID NO: 3051) | | | | | |
| TCR446 | EVDPIGHLY (SEQ | A0101 | 3332 | 3636 | 3942 | 4249 |
| | ID NO: 3051) | | | | | |
| TCR447 | EVDPIGHLY (SEQ | A0101 | 3333 | 3637 | 3943 | 4250 |
| | ID NO: 3051) | | | | | |
| TCR448 | EVDPIGHLY (SEQ | A0101 | 3334 | 3638 | 3944 | 4251 |
| | ID NO: 3051) | | | | | |
| TCR449 | EVDPIGHLY (SEQ | A0101 | 3335 | 3639 | 3945 | 4252 |
| | ID NO: 3051) | | | | | |
| TCR450 | EVDPIGHLY (SEQ | A0101 | 3336 | 3640 | 3946 | 4253 |
| | ID NO: 3051) | | | | | |
| TCR45I | EVDPIGHLY (SEQ | A0101 | 3337 | 3641 | 3947 | 4254 |
| | ID NO: 3051) | | | | | |
| TCR452 | EVDPIGHLY (SEQ | A0101 | 3338 | 3642 | 3948 | 4255 |
| | ID NO: 3051) | | | | | |
| TCR453 | EVDPIGHLY (SEQ | A0101 | 3290 | 3596 | 3900 | 4209 |
| | ID NO: 3051) | | | | | |
| TCR454 | EVDPIGHLY (SEQ | A0101 | 3339 | 3643 | 3949 | 4256 |
| | ID NO: 3051) | | | | | |
| TCR455 | EVDPIGHLY (SEQ | A0101 | 3290 | 3601 | 3900 | 4214 |
| | ID NO: 3051) | | | | | |
| TCR456 | EVDPIGHLY (SEQ | A0101 | 3340 | 3644 | 3950 | 4257 |
| | ID NO: 3051) | | | | | |
| TCR457 | EVDPIGHLY (SEQ | A0101 | 3289 | 3611 | 3899 | 4224 |
| | ID NO: 3051) | | | | | |
| TCR458 | EVDPIGHLY (SEQ | A0101 | 3341 | 3645 | 3951 | 4258 |
| | ID NO: 3051) | | | | | |
| TCR459 | EVDPIGHLY (SEQ | A0101 | 3342 | 3646 | 3952 | 4259 |
| | ID NO: 3051) | | | | | |
| TCR460 | EVDPIGHLY (SEQ | A0101 | 3343 | 3647 | 3953 | 4260 |
| | ID NO: 3051) | | | | | |
| TCR461 | EVDPIGHLY (SEQ | A0101 | 3142 | 3648 | 3751 | 4261 |
| | ID NO: 3051) | | | | | |
| TCR462 | EVDPIGHLY (SEQ | A0101 | 3344 | 3649 | 3954 | 4262 |
| | ID NO: 3051) | | | | | |
| TCR463 | EVDPIGHLY (SEQ | A0101 | 3345 | 3650 | 3955 | 4263 |
| | ID NO: 3051) | | | | | |
| TCR464 | EVDPIGHLY (SEQ | A0101 | 3290 | 3614 | 3956 | 4264 |
| | ID NO: 3051) | | | | | |
| TCR465 | EVDPIGHLY (SEQ | A0101 | 3346 | 3651 | 3957 | 4265 |
| | ID NO: 3051) | | | | | |
| TCR466 | EVDPIGHLY (SEQ | A0101 | 3347 | 3652 | 3958 | 4266 |
| | ID NO: 3051) | | | | | |
| TCR467 | EVDPIGHLY (SEQ | A0101 | 3348 | 3653 | 3959 | 4267 |
| | ID NO: 3051) | | | | | |
| TCR468 | EVDPIGHLY (SEQ | A0101 | 3349 | 3654 | 3960 | 4268 |
| | ID NO: 3051) | | | | | |
| TCR469 | EVDPIGHLY (SEQ | A0101 | 3350 | 3655 | 3961 | 4269 |
| | ID NO: 3051) | | | | | |

TABLE 12
CDR3 and V(D)J sequences of TCR clonotypes confirmed through
cloning

| TCR | | | | | FULL LENGTH | FULL LENGTH |
|---|---|---|---|---|---|---|
| Clonotype ID | | | ALPHA CDR3 | BETA CDR3 | ALPHA VJ (SEQ ID | BETA V(D)J (SEQ |
| # | PEPTIDE | HLA | (SEQ ID NO) | (SEQ ID NO) | NO) | ID NO) |
| TCR2 | EVDPIGHLY (SEQ | A0101 | 4273 | 4287 | 4302 | 4317 |
| | ID NO: 3051) | | | | | |
| TCR4 | EVDPIGHLY (SEQ | A0101 | 4274 | 4288 | 4303 | 4318 |
| | ID NO: 3051) | | | | | |
| TCR53 | EVDPIGHLY (SEQ | A0101 | 4275 | 4289 | 4304 | 4319 |
| | ID NO: 3051) | | | | | |
| TCR54 | EVDPIGHLY (SEQ | A0101 | 4276 | 4290 | 4305 | 4320 |
| | ID NO: 3051) | | | | | |
| TCR19 | LLASSILCA (SEQ | A0201 | 4277 | 4291 | 4306 | 4321 |
| | ID NO: 4270) | | | | | |
| TCR21 | LLASSILCA (SEQ | A0201 | 4278 | 4292 | 4307 | 4322 |
| | ID NO: 4270) | | | | | |
| TCR22 | LLASSILCA (SEQ | A0201 | 4279 | 4293 | 4308 | 4323 |
| | ID NO: 4270) | | | | | |
| TCR18 | LLASSILCA (SEQ | A0201 | 4280 | 4294 | 4309 | 4324 |
| | ID NO: 4270) | | | | | |
| TCR23 | LLASSILCA (SEQ | A0201 | 4281 | 4295 | 4310 | 4325 |
| | ID NO: 4270) | | | | | |
| TCR26 | GVYDGEEHSV | A0201 | 4282 | 4296 | 4311 | 4326 |
| | (SEQ ID NO: 4271) | | | | | |
| TCR28 | GVYDGEEHSV | A0201 | 4283 | 4297 | 4312 | 4327 |
| | (SEQ ID NO: 4271) | | | | | |
| TCR29 | GEMSSNSTAL | B4402 | 4284 | 4298 | 4313 | 4328 |
| | (SEQ ID NO: 4272) | | | | | |
| TCR30 | GEMSSNSTAL | B4402 | 4285 | 4299 | 4314 | 4329 |
| | (SEQ ID NO: 4272) | | | | | |
| TCR32 | GEMSSNSTAL | B4402 | 4286 | 4300 | 4315 | 4330 |
| | (SEQ ID NO: 4272) | | | | | |
| TCR33 | GEMSSNSTAL | B4402 | 3138 | 4301 | 4316 | 4331 |
| | (SEQ ID NO: 4272) | | | | | |

TABLE A
TABLE A

| Target | HLA allele/peptide complex | SEQ ID NO | Protein Name | Ensembl id | Description |
|---|---|---|---|---|---|
| 1 | HLA-A*01:01_EVDPIGHLY | 1 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 2 | HLA-A*29:02_FVQENYLEY | 2 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 3 | HLA-A*29:02_LVHFLLLKY | 3 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 4 | HLA-B*44:03_MEVDPIGHLY | 4 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 5 | HLA-B*35:01_FPVQATIDF | 5 | DSCR6 | ENSG00000183145 | Protein ripply3 (Down syndrome critical region |
| | | | | | protein 6) |
| 6 | HLA-A*26:01_EVDPIGHLY | 1 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 7 | HLA-A*26:01_EVDPIGHVY | 6 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 8 | HLA-A*29:02_FVQENYLEY | 2 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 9 | HLA-A29:02_LVHFLLLKY | 3 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 10 | HLA-B*44:03_MEVDPIGHVY | 7 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 11 | HLA-C*02:02_AEMLGSVVGNW | 8 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 12 | HLA-C*02:02_AEMLGSVVGNW | 8 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 13 | HLA-A*01:01_EVDPIGHVY | 6 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 14 | HLA-B*44:02_AEMLERVIKNY | 9 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 15 | HLA-B*44:03_AEMLERVIKNY | 9 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 16 | HLA-C*02:02_AEMLERVIKNY | 9 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 17 | HLA-B*44:03_AETSYVKVL | 10 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 18 | HLA-A*02:01_ALLEEEEGV | 11 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 19 | HLA-A*01:01_EVDPASNTY | 12 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 20 | HLA-A*02:07_KVDELAHFL | 13 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 21 | HLA-A*30:02_RQVPGSNPARY | 14 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 22 | HLA-B*35:01_SALPTTISF | 15 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 23 | HLA-B*46:01_SALPTTISF | 15 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 24 | HLA-A*02:07_KVDELAHFLL | 16 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 25 | HLA-A*02:01_ALFGLLVYL | 17 | CLD6 | ENSG00000184697 | Claudin-6 (Skullin) |
| 26 | HLA-A*11:01_STSAPAISR | 18 | CLD6 | ENSG00000184697 | Claudin-6 (Skullin) |
| 27 | HLA-C*02:02_KEVDPASNTY | 19 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 28 | HLA-B*44:02_AEMLGSVIRNF | 20 | MAGAC | ENSG00000213401 | Melanoma-associated antigen 12 (Cancer/testis |
| | | | | | antigen 1.12)(CT1.12)(MAGE-12 antigen) |
| | | | | | (MAGE12F antigen) |
| 29 | HLA-C*02:02_AEMLGSVIRNF | 20 | MAGAC | ENSG00000213401 | Melanoma-associated antigen 12 (Cancer/testis |
| | | | | | antigen 1.12)(CT1.12)(MAGE-12 antigen) |
| | | | | | (MAGE12F antigen) |
| 30 | HLA-A*03:01_GLLGDNQIMPK | 21 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 31 | HLA-B*44:02_AEMLGSVVGNW | 8 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 32 | HLA-B*27:02_PRALVETSY | 22 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 33 | HLA-A*01:01_NTDNNLAVY | 23 | KKLC1 | ENSG00000204019 | Kita-kyushu lung cancer antigen 1 (KK-LC-1) |
| | | | | | (Cancer/testis antigen 83) |
| 34 | HLA-B*44:03_AEMLGSVVGNW | 8 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 35 | HLA-B*44:02_AEMLGSVVGNW | 8 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 36 | HLA-B*44:02_AEMLESVIKNY | 24 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 37 | HLA-B*44:03_AEMLESVIKNY | 24 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 38 | HLA-C*02:02_AEMLESVIKNY | 24 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 39 | HLA-B*44:03_AETSYVKVL | 10 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 40 | HLA-B*18:01_EELSVMEVY | 25 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 41 | HLA-A*26:01_EVYDGREHSAY | 26 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 42 | HLA-A*33:01_EYVIKVSAR | 27 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 43 | HLA-A*02:01_KVLEYVIKV | 28 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 44 | HLA-A*02:07_KVLEYVIKV | 28 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 45 | HLA-A*29:02_LVGFLLLKY | 29 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 46 | HLA-B*35:01_SAFPTTINF | 30 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 47 | HLA-C*02:02_SAFPTTINF | 30 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 48 | HLA-A*29:02_LVHFLLLKY | 3 | MAGAC | ENSG00000213401 | Melanoma-associated antigen 12 (Cancer/testis |
| | | | | | antigen 1.12)(CT1.12)(MAGE-12 antigen) |
| | | | | | (MAGE12F antigen) |
| 49 | HLA-A*30:02_STLPTTINY | 31 | MAGAC | ENSG00000213401 | Melanoma-associated antigen 12 (Cancer/testis |
| | | | | | antigen 1.12)(CT1.12)(MAGE-12 antigen) |
| | | | | | (MAGE12F antigen) |
| 50 | HLA-B*18:01_MEVDPIGHVY | 7 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 51 | HLA-B*51:01_DAAHPGPSV | 32 | PPBN | ENSG00000163286 | Alkaline phosphatase, placental-like (EC 3.1.3.1) |
| | | | | | (ALP-1)(Alkaline phosphatase Nagao isozyme) |
| | | | | | (Germ cell alkaline phosphatase)(GCAP) |
| | | | | | (Placental alkaline phosphatase-like)(PLAP-like) |
| 52 | HLA-B*35:01_FPMGTPDPEY | 33 | PPBN | ENSG00000163286 | Alkaline phosphatase, placental-like (EC 3.1.3.1) |
| | | | | | (ALP-1)(Alkaline phosphatase Nagao isozyme) |
| | | | | | (Germ cell alkaline phosphatase)(GCAP) |
| | | | | | (Placental alkaline phosphatase-like)(PLAP-like) |
| 53 | HLA-A*30:02_RVQHASPAGAY | 34 | PPBN | ENSG00000163286 | Alkaline phosphatase, placental-like (EC 3.1.3.1) |
| | | | | | (ALP-1)(Alkaline phosphatase Nagao isozyme) |
| | | | | | (Germ cell alkaline phosphatase)(GCAP) |
| | | | | | (Placental alkaline phosphatase-like)(PLAP-like) |
| 54 | HLA-A*02:07_SLDPSVTHL | 35 | PPBN | ENSG00000163286 | Alkaline phosphatase, placental-like (EC 3.1.3.1) |
| | | | | | (ALP-1)(Alkaline phosphatase Nagao isozyme) |
| | | | | | (Germ cell alkaline phosphatase)(GCAP) |
| | | | | | (Placental alkaline phosphatase-like)(PLAP-like) |
| 55 | HLA-A*30:02_VQHASPAGAY | 36 | PPBN | ENSG00000163286 | Alkaline phosphatase, placental-like (EC 3.1.3.1) |
| | | | | | (ALP-1)(Alkaline phosphatase Nagao isozyme) |
| | | | | | (Germ cell alkaline phosphatase)(GCAP) |
| | | | | | (Placental alkaline phosphatase-like)(PLAP-like) |
| 56 | HLA-A*30:02_EVDPIGHVY | 6 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 57 | HLA-A*29:02_IFLDFNHFY | 37 | PLCX2 | ENSG00000240891 | PI-PLC X domain-containing protein 2 |
| 58 | HLA-A*29:02_LVQEKYLEY | 38 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 59 | HLA-A*30:02_RQVPDSDPARY | 39 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 60 | HLA-B*44:03_AEMLGSVVGNW | 8 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 61 | HLA-A*02:01_FLWGPRALIET | 40 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 62 | HLA-A*02:01_FLWGPRALVET | 41 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 63 | HLA-B*44:03_AEMLGSVIRNF | 20 | MAGAC | ENSG00000213401 | Melanoma-associated antigen 12 (Cancer/testis |
| | | | | | antigen 1.12)(CT1.12)(MAGE-12 antigen) |
| | | | | | (MAGE12F antigen) |
| 64 | HLA-A*29:02_LVQENYLEY | 42 | MAGAC | ENSG00000213401 | Melanoma-associated antigen 12 (Cancer/testis |
| | | | | | antigen 1.12)(CT1.12)(MAGE-12 antigen) |
| | | | | | (MAGE12F antigen) |
| 65 | HLA-A*26:01_EVLNAVGVY | 43 | MAGC2 | ENSG00000046774 | Melanoma-associated antigen C2 (Cancer/testis |
| | | | | | antigen 10)(CT10)(Hepatocellular carcinoma- |
| | | | | | associated antigen 587)(MAGE-C2 antigen) |
| | | | | | (MAGE-E1 antigen) |
| 66 | HLA-A*29:02_GAVSLLLRY | 44 | Q5T1G4 | ENSG00000204179 | Protein tyrosine phosphatase, non-receptor type |
| | | | | | 20A (Tyrosine-protein phosphatase non-receptor |
| | | | | | type 20)(Fragment) |
| 67 | HLA-A*02:07_LLDPVQRNL | 45 | ZN560 | ENSG00000198028 | Zinc finger protein 560 |
| 68 | HLA-C*04:01_TFDSVAVEF | 46 | ZN560 | ENSG00000198028 | Zinc finger protein 560 |
| 69 | HLA-A*01:01_YSDVMLENY | 47 | ZN560 | ENSG00000198028 | Zinc finger protein 560 |
| 70 | HLA-A*02:07_TLDEKVAEL | 48 | MAGC2 | ENSG00000046774 | Melanoma-associated antigen C2 (Cancer/testis |
| | | | | | antigen 10)(CT10)(Hepatocellular carcinoma- |
| | | | | | associated antigen 587)(MAGE-C2 antigen) |
| | | | | | (MAGE-E1 antigen) |
| 71 | HLA-B*51:01_TAFIGNSI | 49 | CLD6 | ENSG00000184697 | Claudin-6 (Skullin) |
| 72 | HLA-A*11:01_STLPTTINY | 31 | MAGAC | ENSG00000213401 | Melanoma-associated antigen 12 (Cancer/testis |
| | | | | | antigen 1.12)(CT1.12)(MAGE-12 antigen) |
| | | | | | (MAGE12F antigen) |
| 73 | HLA-C*04:01_AFDDIATYF | 50 | SSX1 | ENSG00000126752 | Protein SSX1 (Cancer/testis antigen 5.1)(CT5.1) |
| | | | | | (Synovial sarcoma, X breakpoint 1) |
| 74 | HLA-B*35:01_SPASDAYIVF | 51 | NACA2 | ENSG00000253506 | Nascent polypeptide-associated complex subunit |
| | | | | | alpha-2 (Alpha-NAC-like)(Horn s 2.01)(Nascent |
| | | | | | polypeptide-associated complex subunit alpha- |
| | | | | | like)(NAC-alpha-like) |
| 75 | HLA-A*30:02_VYKSPASDAY | 52 | NACA2 | ENSG00000253506 | Nascent polypeptide-associated complex subunit |
| | | | | | alpha-2 (Alpha-NAC-like)(Hom s 2.01)(Nascent |
| | | | | | polypeptide-associated complex subunit alpha- |
| | | | | | like)(NAC-alpha-like) |
| 76 | HLA-B*44:03_KEVDPASNTY | 19 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 77 | HLA-A*30:02_AMNSLSAMY | 53 | DMBX1 | ENSG00000197587 | Diencephalon/mesencephalon homeobox protein |
| | | | | | 1 (Orthodenticle homolog 3)(Paired-like |
| | | | | | homeobox protein DMBX1) |
| 78 | HLA-A*11:01_ASQEDILLK | 54 | SMC1B | ENSG00000077935 | Structural maintenance of chromosomes protein |
| | | | | | 1B (SMC protein 1B)(SMC-1-beta)(SMC-1B) |
| 79 | HLA-A*11:01_ATMTQQLEK | 55 | SMC1B | ENSG00000077935 | Structural maintenance of chromosomes protein |
| | | | | | 1B (SMC protein 1B)(SMC-1-beta)(SMC-1B) |
| 80 | HLA-B*44:03_EEIGVENIREF | 56 | SMC1B | ENSG00000077935 | Structural maintenance of chromosomes protein |
| | | | | | 1B (SMC protein 1B)(SMC-1-beta)(SMC-1B) |
| 81 | HLA-A*11:01_GTVESISVKK | 57 | SMC1B | ENSG00000077935 | Structural maintenance of chromosomes protein |
| | | | | | 1B (SMC protein 1B)(SMC-1-beta)(SMC-1B) |
| 82 | HLA-A*30:02_STSGELIGEY | 58 | SMC1B | ENSG00000077935 | Structural maintenance of chromosomes protein |
| | | | | | 1B (SMC protein 1B)(SMC-1-beta)(SMC-1B) |
| 83 | HLA-A*24:02_VYIAELEKI | 59 | SMC1B | ENSG00000077935 | Structural maintenance of chromosomes protein |
| | | | | | 1B (SMC protein 1B)(SMC-1-beta)(SMC-1B) |
| 84 | HLA-A*11:01_AGQDLSAYLLK | 60 | ACTL8 | ENSG00000117148 | Actin-like protein 8 (Cancer/testis antigen 57) |
| | | | | | (CT57) |
| 85 | HLA-A*26:01_SVVAHLSTY | 61 | ACTL8 | ENSG00000117148 | Actin-like protein 8 (Cancer/testis antigen 57) |
| | | | | | (CT57) |
| 86 | HLA-A*11:01_AAAGVSSTK | 62 | MAGB2 | ENSG00000099399 | Melanoma-associated antigen B2 (Cancer/testis |
| | | | | | antigen 3.2)(CT3.2)(DSS-AHC critical interval |
| | | | | | MAGE superfamily 6)(DAM6)(MAGE XP-2 |
| | | | | | antigen)(MAGE-B2 antigen) |
| 87 | HLA-A*30:02_KVNPNGHTY | 63 | MAGB2 | ENSG00000099399 | Melanoma-associated antigen B2 (Cancer/testis |
| | | | | | antigen 3.2)(CT3.2)(DSS-AHC critical interval |
| | | | | | MAGE superfamily 6)(DAM6)(MAGE XP-2 |
| | | | | | antigen)(MAGE-B2 antigen) |
| 88 | HLA-B*44:02_AEILESVIRNY | 64 | MAGAA | ENSG00000124260 | Melanoma-associated antigen 10 (Cancer/testis |
| | | | | | antigen 1.10)(CT1.10)(MAGE-10 antigen) |
| 89 | HLA-B*44:03_AEILESVIRNY | 64 | MAGAA | ENSG00000124260 | Melanoma-associated antigen 10 (Cancer/testis |
| | | | | | antigen 1.10)(CT1.10)(MAGE-10 antigen) |
| 90 | HLA-C*02:02_AEILESVIRNY | 64 | MAGAA | ENSG00000124260 | Melanoma-associated antigen 10 (Cancer/testis |
| | | | | | antigen 1.10)(CT1.10)(MAGE-10 antigen) |
| 91 | HLA-A*30:02_RQVPGSDPARY | 65 | MAGAA | ENSG00000124260 | Melanoma-associated antigen 10 (Cancer/testis |
| | | | | | antigen 1.10)(CT1.10)(MAGE-10 antigen) |
| 92 | HLA-C*01:02_SSPSVVASL | 66 | MAGAA | ENSG00000124260 | Melanoma-associated antigen 10 (Cancer/testis |
| | | | | | antigen 1.10)(CT1.10)(MAGE-10 antigen) |
| 93 | HLA-A*01:01_LLDPAQRNLY | 67 | ZN560 | ENSG00000198028 | Zinc finger protein 560 |
| 94 | HLA-A*26:01_EVDPASNTY | 12 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 95 | HLA-A*26:01_FVQENYLEY | 2 | MAGA6 | ENSG00000197172 | Melanoma-associated antigen 6 (Cancer/testis |
| | | | | | antigen 1.6)(CT 1.6)(MAGE-6 antigen) |
| | | | | | (MAGE3B antigen) |
| 96 | HLA-C*02:02_MEVDPIGHVY | 7 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 97 | HLA-C*16:01_SALPTTISF | 15 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 98 | HLA-A*01:01_LTDQPSAY | 68 | V9GYR9 | ENSG00000225362 | Cancer/testis antigen 62 (Fragment) |
| 99 | HLA-C*04:01_AFDVASFL | 69 | STRA8 | ENSG00000146857 | Stimulated by retinoic acid gene 8 protein |
| | | | | | homolog |
| 100 | HLA-A*30:02_SSLPTTMNY | 70 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 101 | HLA-B*44:03_EEIIPLNRIY | 71 | TDRD1 | ENSG00000095627 | Tudor domain-containing protein 1 (Cancer/testis |
| | | | | | antigen 41.1)(CT41.1) |
| 102 | HLA-A*01:01_TSDTDVEVLY | 72 | TDRD1 | ENSG00000095627 | Tudor domain-containing protein 1 (Cancer/testis |
| | | | | | antigen 41.1)(CT41.1) |
| 103 | HLA-A*29:02_TFQDILLEARY | 73 | DMBX1 | ENSG00000197587 | Diencephalon/mesencephalon homeobox protein |
| | | | | | 1 (Orthodenticle homolog 3)(Paired-like |
| | | | | | homeobox protein DMBX1) |
| 104 | HLA-A*29:02_YFVQENYLEY | 74 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 105 | HLA-A*29:02_WVQENYLEY | 75 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 106 | HLA-A*33:01_EVRDYVEER | 76 | GBG1 | ENSG00000127928 | Guanine nucleotide-binding protein G(T) subunit |
| | | | | | gamma-T1 (Transducin gamma chain) |
| 107 | HLA-C*16:01_SAFPTTINF | 30 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 108 | HLA-A*02:07_ALFGLLVYL | 17 | CLD6 | ENSG00000184697 | Claudin-6 (Skullin) |
| 109 | HLA-A*02:01_VLTSGIVFV | 77 | CLD6 | ENSG00000184697 | Claudin-6 (Skullin) |
| 110 | HLA-B*35:01_FVQENYLEY | 2 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 111 | HLA-B*51:01_NALSSLKI | 78 | CX7B2 | ENSG00000170516 | Cytochrome c oxidase subunit 7B2, |
| | | | | | mitochondrial (Cytochrome c oxidase |
| | | | | | polypeptide VIIb2) |
| 112 | HLA-B*35:01_NAVLASGTAF | 79 | CX7B2 | ENSG00000170516 | Cytochrome c oxidase subunit 7B2, |
| | | | | | mitochondrial (Cytochrome c oxidase |
| | | | | | polypeptide VIIb2) |
| 113 | HLA-B*27:02_PRALVETSY | 22 | MAGAC | ENSG00000213401 | Melanoma-associated antigen 12 (Cancer/testis |
| | | | | | antigen 1.12)(CT1.12)(MAGE-12 antigen) |
| | | | | | (MAGE12F antigen) |
| 114 | HLA-A*11:01_ASAPPQKQK | 80 | PABP3 | ENSG00000151846 | Polyadenylate-binding protein 3 (PABP-3) |
| | | | | | (Poly(A)-binding protein 3)(Testis-specific |
| | | | | | poly(A)-binding protein) |
| 115 | HLA-A*01:01_EIDNSELLY | 81 | PABP3 | ENSG00000151846 | Polyadenylate-binding protein 3 (PABP-3) |
| | | | | | (Poly(A)-binding protein 3)(Testis-specific |
| | | | | | poly(A)-binding protein) |
| 116 | HLA-A*02:07_KVDEAVAVL | 82 | PABP3 | ENSG00000151846 | Polyadenylate-binding protein 3 (PABP-3) |
| | | | | | (Poly(A)-binding protein 3)(Testis-specific |
| | | | | | poly(A)-binding protein) |
| 117 | HLA-A*33:01_NGKQIYVGR | 83 | PABP3 | ENSG00000151846 | Polyadenylate-binding protein 3 (PABP-3) |
| | | | | | (Poly(A)-binding protein 3)(Testis-specific |
| | | | | | poly(A)-binding protein) |
| 118 | HLA-B*51:01_SPAGPILSI | 84 | PABP3 | ENSG00000151846 | Polyadenylate-binding protein 3 (PABP-3) |
| | | | | | (Poly(A)-binding protein 3)(Testis-specific |
| | | | | | poly(A)-binding protein) |
| 119 | HLA-B*35:01_LPYASTLGY | 85 | SOX14 | ENSG00000168875 | Transcription factor SOX-14 (Protein SOX-28) |
| 120 | HLA-A*26:01_DVADKLVTF | 86 | TDRD1 | ENSG00000095627 | Tudor domain-containing protein 1 (Cancer/testis |
| | | | | | antigen 41.1)(CT41.1) |
| 121 | HLA-A*11:01_GTVESISVK | 87 | SMC1B | ENSG00000077935 | Structural maintenance of chromosomes protein |
| | | | | | 1B (SMC protein 1B)(SMC-1-beta)(SMC-1B) |
| 122 | HLA-B*18:01_IENQAVPAF | 88 | X6RD31 | ENSG00000234068 | P antigen family member 2 (Fragment) |
| 123 | HLA-A*03:01_AVLQKFLEH | 89 | MSLNL | ENSG00000162006 | Mesothelin-like protein (Pre-pro-megakaryocyte- |
| | | | | | potentiating-factor-like) |
| 124 | HLA-B*35:01_QPAAPGPAL | 90 | MSLNL | ENSG00000162006 | Mesothelin-like protein (Pre-pro-megakaiyocyte- |
| | | | | | potentiating-factor-like) |
| 125 | HLA-B*18:01_DEDQAMRAF | 91 | NALP7 | ENSG00000167634 | NACHT, LRR and PYD domains-containing |
| | | | | | protein 7 (Nucleotide-binding oligomerization |
| | | | | | domain protein 12)(PYRIN-containing APAE1- |
| | | | | | like protein 3) |
| 126 | HLA-B*35:01_NPIGDTGVKF | 92 | NALP7 | ENSG00000167634 | NACHT, LRR and PYD domains-containing |
| | | | | | protein 7 (Nucleotide-binding oligomerization |
| | | | | | domain protein 12)(PYRIN-containing APAE1- |
| | | | | | like protein 3) |
| 127 | HLA-C*05:01_NADLQSEF | 93 | V9GYR9 | ENSG00000225362 | Cancer/testis antigen 62 (Fragment) |
| 128 | HLA-B*44:03_SEVSFLEYY | 94 | PIWL1 | ENSG00000125207 | Piwi-like protein 1 (EC 3.1.26.-) |
| 129 | HLA-A*02:01_SLSNRLYYL | 95 | PIWL1 | ENSG00000125207 | Piwi-like protein 1 (EC 3.I.26.-) |
| 130 | HLA-B*44:02_AEMLESVIKNY | 24 | A0A075B7A9 | ENSG00000267978 | Melanoma-associated antigen 9 (Fragment) |
| 131 | HLA-B*44:03_AEMLESVIKNY | 24 | A0A075B7A9 | ENSG00000267978 | Melanoma-associated antigen 9 (Fragment) |
| 132 | HLA-C*02:02_AEMLESVIKNY | 24 | A0A075B7A9 | ENSG00000267978 | Melanoma-associated antigen 9 (Fragment) |
| 133 | HLA-A*02:01_GVYDGEEHSV | 96 | MAGB2 | ENSG00000099399 | Melanoma-associated antigen B2 (Cancer/testis |
| | | | | | antigen 3.2)(CT3.2)(DSS-AHC critical interval |
| | | | | | MAGE supmfamily 6)(DAM6)(MAGE XP-2 |
| | | | | | antigen)(MAGE-B2 antigen) |
| 134 | HLA-B*51:01_DANFIPTV | 97 | V9GZ46 | ENSG00000124092 | Transcriptional repressor CTCFL |
| 135 | HLA-A*30:02_SSLPTTMNY | 70 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 136 | HLA-B*44:03_EELGVMGVY | 98 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 137 | HLA-B*51:01_YPREGSEV | 99 | FOXI3 | ENSG00000214336 | Forkhead box protein I3 |
| 138 | HLA-B*27:02_GLLGDNQIMPK | 21 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 139 | HLA-A*11:01_ATDLHGVSR | 100 | LGSN | ENSG00000146166 | Lengsin (Glutamate-ammonia ligase domain- |
| | | | | | containing protein 1)(Lens glutamine synthase- |
| | | | | | like) |
| 140 | HLA-B*44:02_TEQLTITGKKW | 101 | LGSN | ENSG00000146166 | Lengsin (Glutamate-ammonia ligase domain- |
| | | | | | containing protein 1)(Lens glutamine synthase- |
| | | | | | like) |
| 141 | HLA-B*44:03_TEQLTITGKKW | 101 | LGSN | ENSG00000146166 | Lengsin (Glutamate-ammonia ligase domain- |
| | | | | | containing protein 1)(Lens glutamine synthase- |
| | | | | | like) |
| 142 | HLA-B*44:02_AEIEPVSAVW | 102 | ZFP42 | ENSG00000179059 | Zinc finger protein 42 homolog (Zfp-42) |
| | | | | | (Reduced expression protein 1)(REX-1)(hREX- |
| | | | | | 1)(Zinc finger protein 754) |
| 143 | HLA-B*44:03_AEIEPVSAVW | 102 | ZFP42 | ENSG00000179059 | Zinc finger protein 42 homolog (Zfp-42) |
| | | | | | (Reduced expression protein 1)(REX-1)(hREX- |
| | | | | | 1)(Zinc finger protein 754) |
| 144 | HLA-C*02:02_AEIEPVSAVW | 102 | ZFP42 | ENSG00000179059 | Zinc finger protein 42 homolog (Zfp-42) |
| | | | | | (Reduced expression protein 1)(REX-1)(hREX- |
| | | | | | 1)(Zinc finger protein 754) |
| 145 | HLA-B*44:02_SEQQLSQKVF | 103 | ZFP42 | ENSG00000179059 | Zinc finger protein 42 homolog (Zfp-42) |
| | | | | | (Reduced expression protein 1)(REX-1)(hREX- |
| | | | | | 1)(Zinc finger protein 754) |
| 146 | HLA-B*44:03_SEQQLSQKVF | 103 | ZFP42 | ENSG00000179059 | Zinc finger protein 42 homolog (Zfp-42) |
| | | | | | (Reduced expression protein 1)(REX-1)(hREX- |
| | | | | | 1)(Zinc finger protein 754) |
| 147 | HLA-C*02:02_SEQQLSQKVF | 103 | ZFP42 | ENSG00000179059 | Zinc finger protein 42 homolog (Zfp-42) |
| | | | | | (Reduced expression protein 1)(REX-1)(hREX- |
| | | | | | 1)(Zinc finger protein 754) |
| 148 | HLA-A*01:01_LLDPVQRNLY | 104 | ZN560 | ENSG00000198028 | Zinc finger protein 560 |
| 149 | HLA-A*29:02_LYSDINITY | 105 | NPFF2 | ENSG00000056291 | Neuropeptide FF receptor 2 (G-protein coupled |
| | | | | | receptor 74)(G-protein coupled receptor |
| | | | | | HLWAR77)(Neuropeptide G-protein coupled |
| | | | | | receptor) |
| 150 | HLA-C*05:01_YADLSPNEL | 106 | NPFF2 | ENSG00000056291 | Neuropeptide FF receptor 2 (G-protein coupled |
| | | | | | receptor 74)(G-protein coupled receptor |
| | | | | | HLWAR77)(Neuropeptide G-protein coupled |
| | | | | | receptor) |
| 151 | HLA-B*51:01_YPFKPKLTI | 107 | NPFF2 | ENSG00000056291 | Neuropeptide FF receptor 2 (G-protein coupled |
| | | | | | receptor 74)(G-protein coupled receptor |
| | | | | | HLWAR77)(Neuropeptide G-protein coupled |
| | | | | | receptor) |
| 152 | HLA-A*01:01_YSDINITYVNY | 108 | NPFF2 | ENSG00000056291 | Neuropeptide FF receptor 2 (G-protein coupled |
| | | | | | receptor 74)(G-protein coupled receptor |
| | | | | | HLWAR77)(Neuropeptide G-protein coupled |
| | | | | | receptor) |
| 153 | HLA-A*01:01_YSDINITY | 109 | NPFF2 | ENSG00000056291 | Neuropeptide FF receptor 2 (G-protein coupled |
| | | | | | receptor 74)(G-protein coupled receptor |
| | | | | | HLWAR77)(Neuropeptide G-protein coupled |
| | | | | | receptor) |
| 154 | HLA-A*31:01_QVKIWFQNR | 110 | NKX12 | ENSG00000229544 | NK1 transcription factor-related protein 2 |
| | | | | | (Homeobox protein SAX-1)(NKX-1.1) |
| 155 | HLA-B*51:01_TPFYAPRL | 111 | NKX12 | ENSG00000229544 | NK1 transcription factor-related protein 2 |
| | | | | | (Homeobox protein SAX-1)(NKX-1.1) |
| 156 | HLA-A*11:01_GTMKIHILQK | 112 | V9GZ46 | ENSG00000124092 | Transcriptional repressor CTCFL |
| 157 | HLA-A*29:02_SYFITSLSY | 113 | NPBW1 | ENSG00000183729 | Neuropeptides B/W receptor type 1 (G-protein |
| | | | | | coupled receptor 7) |
| 158 | HLA-B*35:01_SALDESNTY | 114 | ACTL8 | ENSG00000117148 | Actin-like protein 8 (Cancer/testis antigen 57) |
| | | | | | (CT57) |
| 159 | HLA-B*18:01_IESEPLFTY | 115 | MAGC1 | ENSG00000155495 | Melanoma-associated antigen C1 (Cancer/testis |
| | | | | | antigen 7.1)(CT7.1)(MAGE-C1 antigen) |
| 160 | HLA-B*18:01_DEALGGTAF | 116 | TERT | ENSG00000164362 | Telomerase reverse transcriptase (EC 2.7.7.49) |
| | | | | | (HEST2)(Telomerase catalytic subunit) |
| | | | | | (Telomerase-associated protein 2)(TP2) |
| 161 | HLA-A*26:01_EVQSDYSSY | 117 | TERT | ENSG00000164362 | Telomerase reverse transcriptase (EC 2.7.7.49) |
| | | | | | (HEST2)(Telomerase catalytic subunit) |
| | | | | | (Telomerase-associated protein 2)(TP2) |
| 162 | HLA-A*29:02_GLFDVFLRF | 118 | TERT | ENSG00000164362 | Telomerase reverse transcriptase (EC 2.7.7.49) |
| | | | | | (HEST2)(Telomerase catalytic subunit) |
| | | | | | (Telomerase-associated protein 2)(TP2) |
| 163 | HLA-C*01:02_VVIEQSSSL | 119 | TERT | ENSG00000164362 | Telomerase reverse transcriptase (EC 2.7.7.49) |
| | | | | | (HEST2)(Telomerase catalytic subunit) |
| | | | | | (Telomerase-associated protein 2)(TP2) |
| 164 | HLA-A*11:01_SALGVTITK | 120 | ROP1A | ENSG00000065371 | Ropporin-1A (Cancer/testis antigen 91)(CT91) |
| | | | | | (Rhophilin-associated protein 1A) |
| 165 | HLA-A*11:01_GTASLTLPPK | 121 | CA094 | ENSG00000142698 | Uncharacterized protein C1orf94 |
| 166 | HLA-B*44:03_VEVDGPELKF | 122 | CA094 | ENSG00000142698 | Uncharacterized protein C1orf94 |
| 167 | HLA-B*51:01_DAYKFAADV | 123 | BRDT | ENSG00000137948 | Bromodomain testis-specific protein |
| | | | | | (Cancer/testis antigen 9)(CT9)(RING3-like |
| | | | | | protein) |
| 168 | HLA-A*33:01_DVNNQLNSR | 124 | BRDT | ENSG00000137948 | Bromodomain testis-specific protein |
| | | | | | (Cancer/testis antigen 9)(CT9)(RING3-like |
| | | | | | protein) |
| 169 | HLA-B*35:01_IPIEPVESM | 125 | BRDT | ENSG00000137948 | Brodomain testis-specific protein |
| | | | | | (Cancer/testis antigen 9)(CT9)(RING3-like |
| | | | | | protein) |
| 170 | HLA-A*11:01_SSQTAAQVTK | 126 | BRDT | ENSG00000137948 | Brodomain testis-specific protein |
| | | | | | (Cancer/testis antigen 9)(CT9)(RING3-like |
| | | | | | protein) |
| 171 | HLA-C*01:02_QSPQGASAL | 127 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 172 | HLA-B*46:01_AVLASGTAF | 128 | CX7B2 | ENSG00000170516 | Cytochrome c oxidase subunit 7B2, |
| | | | | | mitochondrial (Cytochrome c oxidase |
| | | | | | polypeptide VIIb2) |
| 173 | HLA-A*26:01_EVVEGKEWGSF | 129 | NPSR1 | ENSG00000187258 | Neuropeptide S receptor (G-protein coupled |
| | | | | | receptor 154)(G-protein coupled receptor |
| | | | | | PGR14)(G-protein coupled receptor for asthma |
| | | | | | susceptibility) |
| 174 | HLA-A*01:01_NSAINPLIY | 130 | NPSR1 | ENSG00000187258 | Neuropeptide S receptor (G-protein coupled |
| | | | | | receptor 154)(G-protein coupled receptor |
| | | | | | PGR14)(G-protein coupled receptor for asthma |
| | | | | | susceptibility) |
| 175 | HLA-A*29:02_RYLQVVLLY | 131 | NPSR1 | ENSG00000187258 | Neuropeptide S receptor (G-protein coupled |
| | | | | | receptor 154)(G-protein coupled receptor |
| | | | | | PGR14)(G-protein coupled receptor for asthma |
| | | | | | susceptibility) |
| 176 | HLA-A*29:02_FQNPHGETLLY | 132 | NPFF2 | ENSG00000056291 | Neuropeptide FF receptor 2 (G-protein coupled |
| | | | | | receptor 74)(G-protein coupled receptor |
| | | | | | HLWAR77)(Neuropeptide G-protein coupled |
| | | | | | receptor) |
| 177 | HLA-A*11:01_SVMDLVGSILK | 133 | LDHC | ENSG00000166796 | L-lactate dehydrogenase C chain (LDH-C)(EC |
| | | | | | 1.1.1.27)(Cancer/testis antigen 32)(CT32)(LDH |
| | | | | | testis subunit)(LDH-X) |
| 178 | HLA-A*33:01_EVRDMSEHVTR | 134 | PAGE5 | ENSG00000158639 | P antigen family member 5 (PAGE-5) |
| | | | | | (Cancer/testis antigen 16.1)(CT16.1)(G antigen |
| | | | | | family E member 1)(Prostate-associated gene 5 |
| | | | | | protein) |
| 179 | HLA-A*30:02_AALASVGHLY | 135 | ONEC3 | ENSG00000205922 | One cut domain family member 3 (One cut |
| | | | | | homeobox 3)(Transcription factor ONECUT-3) |
| | | | | | (OC-3) |
| 180 | HLA-A*01:01_VTESESGSPEY | 136 | PPBN | ENSG00000163286 | Alkaline phosphatase, placental-like (EC 3.1.3.1) |
| | | | | | (ALP-1)(Alkaline phosphatase Nagao isozyme) |
| | | | | | (Germ cell alkaline phosphatase)(GCAP) |
| | | | | | (Placental alkaline phosphatase-like)(PLAP-like) |
| 181 | HLA-A*02:07_SIDWFMVTV | 137 | PLAC1 | ENSG00000170965 | Placenta-specific protein 1 |
| 182 | HLA-B*18:01_LEEEVVTF | 138 | TDRD1 | ENSG00000095627 | Tudor domain-containing protein 1 (Cancer/testis |
| | | | | | antigen 41.1)(CT41.1) |
| 183 | HLA-A*26:01_FVQENYLEY | 2 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 184 | HLA-A*29:02_LVQEKYLEY | 38 | MAGB2 | ENSG00000099399 | Melanoma-associated antigen B2 (Cancer/testis |
| | | | | | antigen 3.2)(CT3.2)(DSS-AHC critical interval |
| | | | | | MAGE supeifamily 6)(DAM6)(MAGE XP-2 |
| | | | | | antigen)(MAGE-B2 antigen) |
| 185 | HLA-B*35:01_NASGPDPAL | 139 | NPBW1 | ENSG00000183729 | Neuropeptides B/W receptor type 1 (G-protein |
| | | | | | coupled receptor 7) |
| 186 | HLA-C*01:02_QSPQGASSL | 140 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 187 | HLA-B*44:03_SESEMFPKF | 141 | BRDT | ENSG00000137948 | Brodomain testis-specific protein |
| | | | | | (Cancer/testis antigen 9)(CT9)(RING3-like |
| | | | | | protein) |
| 188 | HLA-A*29:02_NFQGIRFHY | 142 | FATE1 | ENSG00000147378 | Fetal and adult testis-expressed transcript protein |
| | | | | | (Cancer/testis antigen 43)(CT43)(Tumor antigen |
| | | | | | BJ-HCC-2) |
| 189 | HLA-B*51:01_DAAVTHSI | 143 | SAGE1 | ENSG00000181433 | Sarcoma antigen 1 (Cancer/testis antigen 14) |
| | | | | | (CT14) |
| 190 | HLA-A*24:02_LYKPDSNEF | 144 | SAGE1 | ENSG00000181433 | Sarcoma antigen 1 (Cancer/testis antigen 14) |
| | | | | | (CT14) |
| 191 | HLA-B*35:01_MAAAGIPSM | 145 | SAGE1 | ENSG00000181433 | Sarcoma antigen 1 (Cancer/testis antigen 14) |
| | | | | | (CT14) |
| 192 | HLA-B*35:01_MAAGGIPSM | 146 | SAGE1 | ENSG00000181433 | Sarcoma antigen 1 (Cancer/testis antigen 14) |
| | | | | | (CT14) |
| 193 | HLA-B*35:01_MAATPIPAM | 147 | SAGE1 | ENSG00000181433 | Sarcoma antigen 1 (Cancer/testis antigen 14) |
| | | | | | (CT14) |
| 194 | HLA-B*44:03_NEFAVGTKNY | 148 | SAGE1 | ENSG00000181433 | Sarcoma antigen 1 (Cancer/testis antigen 14) |
| | | | | | (CT14) |
| 195 | HLA-C*02:02_NEFAVGTKNY | 148 | SAGE1 | ENSG00000181433 | Sarcoma antigen 1 (Cancer/testis antigen 14) |
| | | | | | (CT14) |
| 196 | HLA-A*24:02_QYAAVTHNI | 149 | SAGE1 | ENSG00000181433 | Sarcoma antigen 1 (Cancer/testis antigen 14) |
| | | | | | (CT14) |
| 197 | HLA-A*03:01_SLFRAVITK | 150 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 198 | HLA-A*29:02_FFLPVSVVY | 151 | NMUR2 | ENSG00000132911 | Neuromectin-U receptor 2 (NMU-R2)(G-protein |
| | | | | | coupled receptor FM-4)(G-protein coupled |
| | | | | | receptor TGR-1) |
| 199 | HLA-A*24:02_IYGFFNENF | 152 | NPFF2 | ENSG00000056291 | Neuropeptide FF receptor 2 (G-protein coupled |
| | | | | | receptor 74)(G-protein coupled receptor |
| | | | | | HLWAR77)(Neuropeptide G-protein coupled |
| | | | | | receptor) |
| 200 | HLA-A*33:01_DYIHKNDNVQR | 153 | PIWL1 | ENSG00000125207 | Piwi-like protein 1 (EC 3.1.26.-) |
| 201 | HLA-C*02:02_SALPTTISF | 15 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 202 | HLA-B*51:01_IPALPLPTI | 154 | DPPA2 | ENSG00000163530 | Developmental pluripotency-associated protein 2 |
| | | | | | (Pluripotent embryonic stem cell-related gene 1 |
| | | | | | protein) |
| 203 | HLA-A*11:01_STSDVKLEK | 155 | DPPA2 | ENSG00000163530 | Developmental pluripotency-associated protein 2 |
| | | | | | (Pluripotent embryonic stem cell-related gene 1 |
| | | | | | protein) |
| 204 | HLA-B*44:03_AEARPVPHW | 156 | ACHA9 | ENSG00000174343 | Neuronal acetylcholine receptor subunit alpha-9 |
| | | | | | (Nicotinic acetylcholine receptor subunit alpha-9) |
| | | | | | (NACHR alpha-9) |
| 205 | HLA-B*35:01_MPAVKNVISY | 157 | ACHA9 | ENSG00000174343 | Neuronal acetylcholine receptor subunit alpha-9 |
| | | | | | (Nicotinic acetylcholine receptor subunit alpha-9) |
| | | | | | (NACHR alpha-9) |
| 206 | HLA-B*46:01_SAFPTTINF | 30 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 207 | HLA-B*35:01_SPFSGGPVSF | 158 | P5F1B | ENSG00000212993 | Putative POU domain, class 5, transcription |
| | | | | | factor 1B (Oct4-pg1)(Octamer-binding protein 3- |
| | | | | | like)(Octamer-binding transcription factor 3-like) |
| 208 | HLA-B*35:01_TAAPATLEL | 159 | MESP2 | ENSG00000188095 | Mesoderm posterior protein 2 (Class C basic |
| | | | | | helix-loop-helix protein 6)(bHLHc6) |
| 209 | HLA-B*46:01_TAAAPGSPF | 160 | NKX12 | ENSG00000229544 | NK1 transcription factor-related protein 2 |
| | | | | | (Homeobox protein SAX-1)(NKX-1.1) |
| 210 | HLA-B*46:01_IAKVTGVAF | 161 | J3KR52 | ENSG00000185055 | EF-hand calcium-binding domain-containing |
| | | | | | protein 10 |
| 211 | HLA-A*11:01_ALAETSYVK | 162 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 212 | HLA-A*30:02_AMIENFNAKY | 163 | NAA11 | ENSG00000156269 | N-alpha-acetyltransferase 11 (EC 2.3.1.255)(N- |
| | | | | | terminal acetyltransferase complex ARD1 subunit |
| | | | | | homolog B)(hARD2)(NatA catalytic subunit |
| | | | | | Naa11) |
| 213 | HLA-B*18:01_DEDGKIVGY | 164 | NAA11 | ENSG00000156269 | N-alpha-acetyltransferase 11 (EC 2.3.1.255)(N- |
| | | | | | terminal acetyltransferase complex ARD1 subunit |
| | | | | | homolog B)(hARD2)(NatA catalytic subunit |
| | | | | | Naa11) |
| 214 | HLA-A*26:01_DVPHGHITSL | 165 | NAA11 | ENSG00000156269 | N-alpha-acetyltransferase 11 (EC 2.3.1.255)(N- |
| | | | | | terminal acetyltransferase complex ARD1 subunit |
| | | | | | homolog B)(hARD2)(NatA catalytic subunit |
| | | | | | Naa11) |
| 215 | HLA-B*35:01_LPENYQMKY | 166 | NAA11 | ENSG00000156269 | N-alpha-acetyltransferase 11 (EC 2.3.1.255)(N- |
| | | | | | temiinal acetyltransferase complex ARD1 subunit |
| | | | | | homolog B)(hARD2)(NatA catalytic subunit |
| | | | | | Naa11) |
| 216 | HLA-A*02:07_ALLEEEEGV | 11 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 217 | HLA-B*51:01_DAVVIALV | 167 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 218 | HLA-A*26:01_EVVGVVYVY | 168 | 56A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 219 | HLA-B*35:01_EVVGVVYVY | 168 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 220 | HLA-B*51:01_FPYLVLTI | 169 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 221 | HLA-A*29:02_ILLFWKPLRY | 170 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 222 | HLA-B*18:01_LEVVGVVY | 171 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 223 | HLA-A*29:02_LLFWKPLRY | 172 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 224 | HLA-B*51:01_LPQTPLVI | 173 | NPBW1 | ENSG00000183729 | Neuropeptides B/W receptor type 1 (G-protein |
| | | | | | coupled receptor 7) |
| 225 | HLA-B*46:01_VAIDQYNTF | 174 | NPBW1 | ENSG00000183729 | Neuropeptides B/W receptor type 1 (G-protein |
| | | | | | coupled receptor 7) |
| 226 | HLA-A*01:01_ITDPTDPVDY | 175 | DCC | ENSG00000187323 | Netrin receptor DCC (Colorectal cancer |
| | | | | | suppressor)(Immunoglobulin superfamily DCC |
| | | | | | subclass member 1)(Tumor suppressor protein |
| | | | | | DCC) |
| 227 | HLA-A*02:07_LLPASSFSV | 176 | DCC | ENSG00000187323 | Netrin receptor DCC (Colorectal cancer |
| | | | | | suppressor)(Immunoglobulin superfamily DCC |
| | | | | | subclass member 1)(Tumor suppressor protein |
| | | | | | DCC) |
| 228 | HLA-A*02:01_SIWEGLVTV | 177 | DCC | ENSG00000187323 | Netrin receptor DCC (Colorectal cancer |
| | | | | | suppressor)(Immunoglobulin superfamily DCC |
| | | | | | subclass member 1)(Tumor suppressor protein |
| | | | | | DCC) |
| 229 | HLA-A*30:02_IVNPPPPEY | 178 | BRDT | ENSG00000137948 | Brodomain testis-specific protein |
| | | | | | (Cancer/testis antigen 9)(CT9)(RING3-like |
| | | | | | protein) |
| 230 | HLA-A*02:01_FLAPLSFYL | 179 | ACHA9 | ENSG00000174343 | Neuronal acetylcholine receptor subunit alpha-9 |
| | | | | | (Nicotinic acetylcholine receptor subunit alpha-9) |
| | | | | | (NACHR alpha-9) |
| 231 | HLA-A*29:02_VNTNVVLRY | 180 | ACHA9 | ENSG00000174343 | Neuronal acetylcholine receptor subunit alpha-9 |
| | | | | | (Nicotinic acetylcholine receptor subunit alpha-9) |
| | | | | | (NACHR alpha-9) |
| 232 | HLA-B*18:01_NEFAVGTKNY | 148 | SAGE1 | ENSG00000181433 | Sarcoma antigen 1 (Cancer/testis antigen 14) |
| | | | | | (CT14) |
| 233 | HLA-A*11:01_ASVEASKLK | 181 | V9GZ46 | ENSG00000124092 | Transcriptional repressor CTCFL |
| 234 | HLA-A*26:01_EVISVQMSM | 182 | UROL1 | ENSG00000177398 | Uromodulin-like 1 (Olfactorin) |
| 235 | HLA-B*35:01_TATLLIVRY | 183 | UROL1 | ENSG00000177398 | Uromodulin-like 1 (Olfactorin) |
| 236 | HLA-B*44:02_TEDPTGHFLW | 184 | UROL1 | ENSG00000177398 | Uromodulin-like 1 (Olfactorin) |
| 237 | HLA-B*44:03_TEDPTGHFLW | 184 | UROL1 | ENSG00000177398 | Uromodulin-like 1 (Olfactorin) |
| 238 | HLA-B*08:01_TIKTKYVL | 185 | UROL1 | ENSG00000177398 | Uromodulin-like 1 (Olfactorin) |
| 239 | HLA-B*18:01_YEVISVQM | 186 | UROL1 | ENSG00000177398 | Uromodulin-like 1 (Olfactorin) |
| 240 | HLA-A*02:07_ALDPPVDVFV | 187 | LN28A | ENSG00000131914 | Protein lin-28 homolog A (Lin-28A)(Zinc finger |
| | | | | | CCHC domain-containing protein 1) |
| 241 | HLA-B*35:01_HAGEDVAVF | 188 | PPBN | ENSG00000163286 | Alkaline phosphatase, placental-like (EC 3.1.3.1) |
| | | | | | (ALP-1)(Alkaline phosphatase Nagao isozyme) |
| | | | | | (Germ cell alkaline phosphatase)(GCAP) |
| | | | | | (Placental alkaline phosphatase-like)(PLAP-like) |
| 242 | HLA-C*02:02_AETSYVKVL | 10 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 243 | HLA-A*29:02_AFGDILHRY | 189 | TRI51 | ENSG00000124900 | Tripartite motif-containing protein 51 (SPRY |
| | | | | | domain-containing protein 5) |
| 244 | HLA-A*26:01_ENVPLIGKY | 190 | ACHA9 | ENSG00000174343 | Neuronal acetylcholine receptor subunit alpha-9 |
| | | | | | (Nicotinic acetylcholine receptor subunit alpha-9) |
| | | | | | (NACHR alpha-9) |
| 245 | HLA-A*11:01_SSIFGLAPGK | 191 | PPBN | ENSG00000163286 | Alkaline phosphatase, placental-like (EC 3.1.3.1) |
| | | | | | (ALP-1)(Alkaline phosphatase Nagao isozyme) |
| | | | | | (Germ cell alkaline phosphatase)(GCAP) |
| | | | | | (Placental alkaline phosphatase-like)(PLAP-like) |
| 246 | HLA-B*51:01_LPTDLFNSV | 192 | ROP1A | ENSG00000065371 | Ropporin-1A (Cancer/testis antigen 91)(CT91) |
| | | | | | (Rhophilin-associated protein 1A) |
| 247 | HLA-A*33:01_DTFSYPIER | 193 | ACTL8 | ENSG00000117148 | Actin-like protein 8 (Cancer/testis antigen 57) |
| | | | | | (CT57) |
| 248 | HLA-A*26:01_EVPSGVIPNL | 194 | MAGC2 | ENSG00000046774 | Melanoma-associated antigen C2 (Cancer/testis |
| | | | | | antigen 10)(CT10)(Hepatocellular carcinoma- |
| | | | | | associated antigen 587)(MAGE-C2 antigen) |
| | | | | | (MAGE-E1 antigen) |
| 249 | HLA-A*24:02_LYATVIHDI | 195 | SAGE1 | ENSG00000181433 | Sarcoma antigen 1 (Cancer/testis antigen 14) |
| | | | | | (CT14) |
| 250 | HLA-B*35:01_FVQENYLEY | 2 | MAGA3 | ENSG00000221867 | Melanoma-associated antigen 3 (Antigen MZ2- |
| | | | | | D)(Cancer/testis antigen 1.3)(CT1.3)(MAGE-3 |
| | | | | | antigen) |
| 251 | HLA-A*11:01_SSYNRGLISK | 196 | NPSR1 | ENSG00000187258 | Neuropeptide S receptor (G-protein coupled |
| | | | | | receptor 154)(G-protein coupled receptor |
| | | | | | PGR14)(G-protein coupled receptor for asthma |
| | | | | | susceptibility) |
| 252 | HLA-A*02:07_ALDESNTYQL | 197 | ACTL8 | ENSG00000117148 | Actin-like protein 8 (Cancer/testis antigen 57) |
| | | | | | (CT57) |
| 253 | HLA-B*44:02_AEQRDDILYF | 198 | CRLF2 | ENSG00000205755 | Cytokine receptor-like factor 2 (Cytokine |
| | | | | | receptor-like 2)(IL-XR)(Thymic stromal |
| | | | | | lymphopoietin protein receptor)(TSLP receptor) |
| 254 | HLA-B*44:03_AEQRDDILYF | 198 | CRLF2 | ENSG00000205755 | Cytokine receptor-like factor 2 (Cytokine |
| | | | | | receptor-like 2)(IL-XR)(Thymic stromal |
| | | | | | lymphopoietin protein receptor)(TSLP receptor) |
| 255 | HLA-A*02:01_FLWGPRALAET | 199 | MAGA4 | ENSG00000147381 | Melanoma-associated antigen 4 (Cancer/testis |
| | | | | | antigen 1.4)(CT1.4)(MAGE-4 antigen)(MAGE- |
| | | | | | 41 antigen)(MAGE-X2 antigen) |
| 256 | HLA-A*11:01_IVQEPTEEK | 200 | X6RD31 | ENSG00000234068 | P antigen family member 2 (Fragment) |
| 257 | HLA-B*44:03_EESVLVGYVDY | 201 | TDRD1 | ENSG00000095627 | Tudor domain-containing protein 1 (Cancer/testis |
| | | | | | antigen 41.1)(CT41.1) |
| 258 | HLA-B*08:01_EVKARTQEL | 202 | BRDT | ENSG00000137948 | Brodomain testis-specific protein |
| | | | | | (Cancer/testis antigen 9)(CT9)(RING3-like |
| | | | | | protein) |
| 259 | HLA-A*11:01_SSDSESEMFPK | 203 | BRDT | ENSG00000137948 | Brodomain testis-specific protein |
| | | | | | (Cancer/testis antigen 9)(CT9)(RING3-like |
| | | | | | protein) |
| 260 | HLA-B*35:01_DANFIPTVY | 204 | V9GZ46 | ENSG00000124092 | Transcriptional repressor CTCFL |
| 261 | HLA-B*44:03_QESDLRLFL | 205 | NALP7 | ENSG00000167634 | NACHT, LRR and PYD domains-containing |
| | | | | | protein 7 (Nucleotide-binding oligomerization |
| | | | | | domain protein 12)(PYRIN-containing APAF1- |
| | | | | | like protein 3) |
| 262 | HLA-A*01:01_YSEKISYVY | 206 | SSX1 | ENSG00000126752 | Protein SSX1 (Cancer/testis antigen 5.1)(CT5.1) |
| | | | | | (Synovial sarcoma, X breakpoint 1) |
| 263 | HLA-A*03:01_RVHPVSTMVK | 207 | LDHC | ENSG00000166796 | L-lactate dehydrogenase C chain (LDH-C)(EC |
| | | | | | 1.1.1.27)(Cancer/testis antigen 32)(CT32)(LDH |
| | | | | | testis subunit)(LDH-X) |
| 264 | HLA-B*44:03_AEDEDGKIVGY | 208 | NAA11 | ENSG00000156269 | N-alpha-acetyltransferase 11 (EC 2.3.1.255)(N- |
| | | | | | terminal acetyltransferase complex ARD1 subunit |
| | | | | | homolog B)(hARD2)(NatA catalytic subunit |
| | | | | | Naa11) |
| 265 | HLA-B*44:03_EEPLSVTAKY | 209 | VCX1 | ENSG00000182583 | Variable charge X-linked protein 1 (Variable |
| | | | | | charge protein on X with ten repeats)(VCX-10r) |
| | | | | | (Variably charged protein X-B1)(VCX-B1) |
| 266 | HLA-A*29:02_AFLEVVGVVY | 210 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 267 | HLA-A*29:02_ILTDINWRF | 211 | NPSR1 | ENSG00000187258 | Neuropeptide S receptor (G-protein coupled |
| | | | | | receptor 154)(G-protein coupled receptor |
| | | | | | PGR14)(G-protein coupled receptor for asthma |
| | | | | | susceptibility) |
| 268 | HLA-B*18:01_SEDPILTAF | 212 | TRPC5 | ENSG00000072315 | Short transient receptor potential channel 5 |
| | | | | | (TrpC5)(Transient receptor protein 5)(TRP-5) |
| | | | | | (hTRP-5)(hTRP5) |
| 269 | HLA-B*44:03_SEDPILTAF | 212 | TRPC5 | ENSG00000072315 | Short transient receptor potential channel 5 |
| | | | | | (TrpC5)(Transient receptor protein 5)(TRP-5) |
| | | | | | (hTRP-5)(hTRP5) |
| 270 | HLA-B*44:03_EETPFSRLI | 213 | INSL6 | ENSG00000120210 | Insulin-like peptide INSL6 (Insulin-like peptide |
| | | | | | 6)(Relaxin/insulin-like factor 1)[Cleaved into: |
| | | | | | Insulin-like peptide INSL6 B chain; Insulin-like |
| | | | | | peptide INSL6 A chain] |
| 271 | HLA-A*24:02_VYIHENAKF | 214 | INSL6 | ENSG00000120210 | Insulin-like peptide INSL6 (Insulin-like peptide |
| | | | | | 6)(Relaxin/insulin-like factor 1)[Cleaved into: |
| | | | | | Insulin-like peptide INSL6 B chain; Insulin-like |
| | | | | | peptide INSL6 A chain] |
| 272 | HLA-C*01:02_ITPQRQSAL | 215 | TDRD1 | ENSG00000095627 | Tudor domain-containing protein 1 (Cancer/testis |
| | | | | | antigen 41.1)(CT41.1) |
| 273 | HLA-B*51:01_EPYPVTKNI | 216 | CNTP5 | ENSG00000155052 | Contactin-associated protein-like 5 (Cell |
| | | | | | recognition molecule Caspr5) |
| 274 | HLA-B*35:01_IAAAFAVDY | 217 | X6RE50 | ENSG00000004809 | Solute carrier family 22 member 16 (Fragment) |
| 275 | HLA-B*35:01_MPLEVYEM | 218 | NMUR2 | ENSG00000132911 | Neuromeclin-U receptor 2 (NMU-R2)(G-protein |
| | | | | | coupled receptor FM-4)(G-protein coupled |
| | | | | | receptor TGR-1) |
| 276 | HLA-B*44:03_AEATQSMNAKY | 219 | TRIMM | ENSG00000179046 | Probable E3 ubiquitin-protein ligase TRIML2 |
| | | | | | (EC 2.3.2.27)(RING-type E3 ubiquitin |
| | | | | | transferase TRIML2)(SPRY domain-containing |
| | | | | | protein 6)(Tripartite motif family-like protein 2) |
| 277 | HLA-C*02:02_AEATQSMNAKY | 219 | TRIMM | ENSG00000179046 | Probable E3 ubiquitin-protein ligase TRIML2 |
| | | | | | (EC 2.3.2.27)(RING-type E3 ubiquitin |
| | | | | | transferase TRIML2)(SPRY domain-containing |
| | | | | | protein 6)(Tripartite motif family-like protein 2) |
| 278 | HLA-A*30:02_ATQSMNAKY | 220 | TRIMM | ENSG00000179046 | Probable E3 ubiquitin-protein ligase TRIML2 |
| | | | | | (EC 2.3.2.27)(RING-type E3 ubiquitin |
| | | | | | transferase TRIML2)(SPRY domain-containing |
| | | | | | protein 6)(Tripartite motif family-like protein 2) |
| 279 | HLA-A*02:07_KLDTVGVFL | 221 | TRIMM | ENSG00000179046 | Probable E3 ubiquitin-protein ligase TRIML2 |
| | | | | | (EC 2.3.2.27)(RING-type E3 ubiquitin |
| | | | | | transferase TRIML2)(SPRY domain-containing |
| | | | | | protein 6)(Tripartite motif family-like protein 2) |
| 280 | HLA-B*44:03_EEIENLYRF | 222 | MEIG1 | ENSG00000197889 | Meiosis expressed gene 1 protein homolog |
| 281 | HLA-A*29:02_VLLDEGAMLLY | 223 | NALP7 | ENSG00000167634 | NACHT, LRR and PYD domains-containing |
| | | | | | protein 7 (Nucleotide-binding oligomerization |
| | | | | | domain protein 12)(PYRIN-containing APAF1- |
| | | | | | like protein 3) |
| 282 | HLA-B*44:03_EEFQVLVKKI | 224 | S100G | ENSG00000169906 | Protein S100-G (Calbindin-D9k)(S100 calcium- |
| | | | | | binding protein G)(Vitamin D-dependent |
| | | | | | calcium-binding protein, intestinal)(CABP) |
| 283 | HLA-A*02:07_TLDDLFQEL | 225 | S100G | ENSG00000169906 | Protein S100-G (Calbindin-D9k)(S100 calcium- |
| | | | | | binding protein G)(Vitamin D-dependent |
| | | | | | calcium-binding protein, intestinal)(CABP) |
| 284 | HLA-C*04:01_TYDGMLSDV | 226 | MAGAA | ENSG00000124260 | Melanoma-associated antigen 10 (Cancer/testis |
| | | | | | antigen 1.10)(CT1.10)(MAGE-10 antigen) |
| 285 | HLA-B*18:01_TEFVGATM | 227 | TRPC5 | ENSG00000072315 | Short transient receptor potential channels |
| | | | | | (TrpC5)(Transient receptor protein 5)(TRP-5) |
| | | | | | (hTRP-5)(hTRP5) |
| 286 | HLA-C*01:02_QSPQGASSL | 140 | MAGA6 | ENSG00000197172 | Melanoma-associated antigen 6 (Cancer/testis |
| | | | | | antigen 1.6)(CT1.6)(MAGE-6 antigen) |
| | | | | | (MAGE3B antigen) |
| 287 | HLA-B*51:01_IPFTPPTV | 228 | A0A1BOGTJ6 | ENSG00000268655 | HCG1796489 |
| 288 | HLA-A*11:01_TVADPLPQVAK | 229 | A0A1BOGTJ6 | ENSG00000268655 | HCG1796489 |
| 289 | HLA-A*02:01_TVADPLPQV | 230 | A0A1BOGTJ6 | ENSG00000268655 | HCG1796489 |
| 290 | HLA-A*29:02_GYLVVGFVY | 231 | X6RE50 | ENSG00000004809 | Solute carrier family 22 member 16 (Fragment) |
| 291 | HLA-A*30:02_GQNLSIHSGQY | 232 | CRSPL | ENSG00000101074 | Peptidase inhibitor R3HDML (Cysteine-rich |
| | | | | | secretory protein R3HDML) |
| 292 | HLA-A*30:02_SVYPPAANMEY | 233 | CRSPL | ENSG00000101074 | Peptidase inhibitor R3HDML (Cysteine-rich |
| | | | | | secretory protein R3HDML) |
| 293 | HLA-A*11:01_SSSSPISNK | 234 | DMRT1 | ENSG00000137090 | Double sex-and mab-3-related transcription factor |
| | | | | | 1 (DM domain expressed in testis protein 1) |
| 294 | HLA-A*29:02_FLSSLFPFRY | 235 | MAJIN | ENSG00000168070 | Membrane-anchored junction protein |
| 295 | HLA-B*18:01_QELEVGKEAY | 236 | MAJIN | ENSG00000168070 | Membrane-anchored junction protein |
| 296 | HLA-B*44:03_QELEVGKEAY | 236 | MAJIN | ENSG00000168070 | Membrane-anchored junction protein |
| 297 | HLA-C*02:02_QELEVGKEAY | 236 | MAJIN | ENSG00000168070 | Membrane-anchored junction protein |
| 298 | HLA-B*44:02_SEQPPASLGF | 237 | MAJIN | ENSG00000168070 | Membrane-anchored junction protein |
| 299 | HLA-B*44:03_SEQPPASLGF | 237 | MAJIN | ENSG00000168070 | Membrane-anchored junction protein |
| 300 | HLA-C*02:02_SEQPPASLGF | 237 | MAJIN | ENSG00000168070 | Membrane-anchored junction protein |
| 301 | HLA-B*44:03_GELREISGNQY | 238 | PDCL2 | ENSG00000163440 | Phosducin-like protein 2 |
| 302 | HLA-A*26:01_EVKKEYASM | 239 | STRA8 | ENSG00000146857 | Stimulated by retinoic acid gene 8 protein |
| | | | | | homolog |
| 303 | HLA-A*33:01_DYFPVILKR | 240 | MAGC2 | ENSG00000046774 | Melanoma-associated antigen C2 (Cancer/testis |
| | | | | | antigen 10)(CT10)(Hepatocellular carcinoma- |
| | | | | | associated antigen 587)(MAGE-C2 antigen) |
| | | | | | (MAGE-E1 antigen) |
| 304 | HLA-B*44:03_KEGEPVEFIF | 241 | LN28B | ENSG00000187772 | Protein lin-28 homolog B (Lin-28B) |
| 305 | HLA-A*11:01_TVAVTQMNK | 242 | ACTL8 | ENSG00000117148 | Actin-like protein 8 (Cancer/testis antigen 57) |
| | | | | | (CT57) |
| 306 | HLA-A*02:07_VLDEVDAAL | 243 | SMC1B | ENSG00000077935 | Structural maintenance of chromosomes protein |
| | | | | | 1B (SMC protein 1B)(SMC-1-beta)(SMC-1B) |
| 307 | HLA-A*26:01_DVRIEVGLY | 244 | UROL1 | ENSG00000177398 | Uromodulin-like 1 (Olfactorin) |
| 308 | HLA-B*35:01_HPFKPDATY | 245 | PO4F1 | ENSG00000152192 | POU domain, class 4, transcription factor 1 |
| | | | | | (Brain-specific homeobox/POU domain protein |
| | | | | | 3A)(Brain-3A)(Brn-3A)(Homeobox/POU |
| | | | | | domain protein RDC-1)(Oct-T1) |
| 309 | HLA-A*11:01_AVLSAGPIITR | 246 | ZPLD1 | ENSG00000170044 | Zona pellucida-like domain-containing protein 1 |
| | | | | | (ZP domain-containing protein 1) |
| 310 | HLA-A*26:01_DTPDPPTIISY | 247 | ZPLD1 | ENSG00000170044 | Zona pellucida-like domain-containing protein 1 |
| | | | | | (ZP domain-containing protein 1) |
| 311 | HLA-B*35:01_FPAERDISVY | 248 | ZPLD1 | ENSG00000170044 | Zona pellucida-like domain-containing protein 1 |
| | | | | | (ZP domain-containing protein 1) |
| 312 | HLA-A*26:01_STIPGVSAY | 249 | ZPLD1 | ENSG00000170044 | Zona pellucida-like domain-containing protein 1 |
| | | | | | (ZP domain-containing protein 1) |
| 313 | HLA-B*46:01_STIPGVSAY | 249 | ZPLD1 | ENSG00000170044 | Zona pellucida-like domain-containing protein 1 |
| | | | | | (ZP domain-containing protein 1) |
| 314 | HLA-A*30:02_AGMTIATSY | 250 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provims ancestral Env |
| | | | | | polyprotein)[Cleaved into: Surface protein (SU); |
| | | | | | Transmembrane protein (TM)] |
| 315 | HLA-A*33:01_DSLAAVVLQNR | 251 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 316 | HLA-A*03:01_GTGIAGITK | 252 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 317 | HLA-A*11:01_GTGIAGITK | 252 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 318 | HLA-A*11:01_GTGTGIAGITK | 253 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 319 | HLA-B*18:01_IEAELHISY | 254 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 320 | HLA-B*44:03_IEAELHISY | 254 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 321 | HLA-B*51:01_LPLTGPLV | 255 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 322 | HLA-A*01:01_TVDSNQQTY | 256 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 323 | HLA-A*24:02_TYQTYTHNQF | 257 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 324 | HLA-B*51:01_DPFFKQQAV | 258 | ADAM2 | ENSG00000104755 | Disintegrin and metalloproteinase domain- |
| | | | | | containing protein 2 (ADAM 2)(Cancer/testis |
| | | | | | antigen 15)(CT15)(Fertilin subunit beta)(PH- |
| | | | | | 30)(PH30)(PH30-beta) |
| 325 | HLA-B*44:03_GEANELLHTF | 259 | ADAM2 | ENSG00000104755 | Disintegrin and metalloproteinase domain- |
| | | | | | containing protein 2 (ADAM 2)(Cancer/testis |
| | | | | | antigen 15)(CT15)(Fertilin subunit beta)(PH- |
| | | | | | 30)(PH30)(PH30-beta) |
| 326 | HLA-C*04:01_NFDSLPVQI | 260 | ADAM2 | ENSG00000104755 | Disintegrin and metalloproteinase domain- |
| | | | | | containing protein 2 (ADAM 2)(Cancer/testis |
| | | | | | antigen 15)(CT15)(Fertilin subunit beta)(PH- |
| | | | | | 30)(PH30)(PH30-beta) |
| 327 | HLA-A*11:01_ATAQPSQVRQK | 261 | FHL17 | ENSG00000132446 | Ferritin heavy polypeptide-like 17 (Cancer/testis |
| | | | | | antigen 38)(CT38) |
| 328 | HLA-A*11:01_SVLSEQFTK | 262 | V9GZ46 | ENSG00000124092 | Transcriptional repressor CTCFL |
| 329 | HLA-A*33:01_DIQEPYYGR | 263 | I22R2 | ENSG00000164485 | Interleukin-22 receptor subunit alpha-2 (IL-22 |
| | | | | | receptor subunit alpha-2)(IL-22R-alpha-2)(IL- |
| | | | | | 22RA2)(Cytokine receptor class-II member 10) |
| | | | | | (Cytokine receptor family 2 member 10)(CRF2- |
| | | | | | 10)(Cytokine receptor family type 2, soluble 1) |
| | | | | | (CRF2-S1)(Interleukin-22-binding protein)(IL- |
| | | | | | 22BP)(IL22BP)(ZcytoR16) |
| 330 | HLA-A*01:01_TSDIQEPYY | 264 | I22R2 | ENSG00000164485 | Interleukin-22 receptor subunit alpha-2 (IL-22 |
| | | | | | receptor subunit alpha-2)(IL-22R-alpha-2)(IL- |
| | | | | | 22RA2)(Cytokine receptor class-II member 10) |
| | | | | | (Cytokine receptor family 2 member 10)(CRF2- |
| | | | | | 10)(Cytokine receptor family type 2, soluble 1) |
| | | | | | (CRF2-S1)(Interleukin-22-binding protein)(IL- |
| | | | | | 22BP)(IL22BP)(ZcytoR16) |
| 331 | HLA-A*02:07_FLPDAFVTM | 265 | TDT | ENSG00000107447 | DNA nucleotidylexotransferase (EC 2.7.7.31) |
| | | | | | (Terminal addition enzyme)(Terminal |
| | | | | | deoxynucleandyltransferase)(Terminal |
| | | | | | transferase) |
| 332 | HLA-A*29:02_IFAHLGLDY | 266 | TDT | ENSG00000107447 | DNA nucleotidylexotransferase (EC 2.7.7.31) |
| | | | | | (Terminal addition enzyme)(Terminal |
| | | | | | deoxynucleandyltransferuse)(Terminal |
| | | | | | transferase) |
| 333 | HLA-A*02:01_KLFTSVFGV | 267 | TDT | ENSG00000107447 | DNA nucleotidylexotransferase (EC 2.7.7.31) |
| | | | | | (Terminal addition enzyme)(Terminal |
| | | | | | deoxynucleandyltransferuse)(Terminal |
| | | | | | transferase) |
| 334 | HLA-C*04:01_YYDLVESTF | 268 | TDT | ENSG00000107447 | DNA nucleotidylexotransferase (EC 2.7.7.31) |
| | | | | | (Terminal addition enzyme)(Terminal |
| | | | | | deoxynucleandyltransferuse)(Terminal |
| | | | | | transferase) |
| 335 | HLA-B*08:01_YPVTKNISL | 269 | CNTP5 | ENSG00000155052 | Contactin-associated protein-like 5 (Cell |
| | | | | | recognition molecule Caspr5) |
| 336 | HLA-B*18:01_SEVSFLEY | 270 | PIWL1 | ENSG00000125207 | Piwi-like protein 1 (EC 3.1.26.-) |
| 337 | HLA-A*11:01_ITWDAPAITK | 271 | ACHA9 | ENSG00000174343 | Neuronal acetylcholine receptor subunit alpha-9 |
| | | | | | (Nicotinic acetylcholine receptor subunit alpha-9) |
| | | | | | (NACHR alpha-9) |
| 338 | HLA-B*44:03_TEVVEGKEW | 272 | NPSR1 | ENSG00000187258 | Neuropeptide S receptor (G-protein coupled |
| | | | | | receptor 154)(G-protein coupled receptor |
| | | | | | PGR14)(G-protein coupled receptor for asthma |
| | | | | | susceptibility) |
| 339 | HLA-C*02:02_VAIDQYNTF | 174 | NPBW1 | ENSG00000183729 | Neuropeptides B/W receptor type 1 (G-protein |
| | | | | | coupled receptor 7) |
| 340 | HLA-B*35:01_DAWGGNTAY | 273 | CRBA4 | ENSG00000196431 | Beta-crystallin A4 (Beta-A4 crystallin) |
| 341 | HLA-B*08:01_YPAERLTSF | 274 | CRBA4 | ENSG00000196431 | Beta-clystallin A4 (Beta-A4 crystallin) |
| 342 | HLA-B*44:03_EEVSNLVNY | 275 | SUN3 | ENSG00000164744 | SUN domain-containing protein 3 (Sad1/unc-84 |
| | | | | | domain-containing protein 1) |
| 343 | HLA-B*18:01_IEAGTSESY | 276 | SUN3 | ENSG00000164744 | SUN domain-containing protein 3 (Sad1/unc-84 |
| | | | | | domain-containing protein 1) |
| 344 | HLA-B*44:03_IEAGTSESY | 276 | SUN3 | ENSG00000164744 | SUN domain-containing protein 3 (Sad1/unc-84 |
| | | | | | domain-containing protein 1) |
| 345 | HLA-A*29:02_IFSNWGHPKY | 277 | SUN3 | ENSG00000164744 | SUN domain-containing protein 3 (Sad1/unc-84 |
| | | | | | domain-containing protein 1) |
| 346 | HLA-A*11:01_VTMEHISEK | 278 | SUN3 | ENSG00000164744 | SUN domain-containing protein 3 (Sad1/unc-84 |
| | | | | | domain-containing protein 1) |
| 347 | HLA-A*02:01_YLSEALQEA | 279 | NALP7 | ENSG00000167634 | NACHT, LRR and PYD domains-containing |
| | | | | | protein 7 (Nucleotide-binding oligomerization |
| | | | | | domain protein 12)(PYRIN-containing APAF1- |
| | | | | | like protein 3) |
| 348 | HLA-A*26:01_EVVGELVAKF | 280 | TFDP3 | ENSG00000183434 | Transcription factor Dp family member 3 |
| | | | | | (Cancer/testis antigen 30)(CT30)(Hepatocellular |
| | | | | | carcinoma-associated antigen 661) |
| 349 | HLA-A*02:01_GMMDDYTYV | 281 | NKAI3 | ENSG00000185942 | Sodium/potassium-transporting ATPase subunit |
| | | | | | beta-1-interacting protein 3 (Na(+)/K(+)- |
| | | | | | transporting ATPase subunit beta-1-interacting |
| | | | | | protein 3)(Protein FAM77D) |
| 350 | HLA-B*44:03_AEMAVGLVVF | 282 | COX8C | ENSG00000187581 | Cytochrome c oxidase subunit 8C, mitochondrial |
| | | | | | (Cytochrome c oxidase polypeptide 8 isoform 3) |
| | | | | | (Cytochrome c oxidase polypeptide VIII isoform |
| | | | | | 3)(COX VIII-3)(Cytochrome c oxidase subunit |
| | | | | | 8-3)(COX8-3)(Cytochrome c oxidase subunit |
| | | | | | VIIIC) |
| 351 | HLA-C*02:02_AEMAVGLVVF | 282 | COX8C | ENSG00000187581 | Cytochrome c oxidase subunit 8C, mitochondrial |
| | | | | | (Cytochrome c oxidase polypeptide 8 isoform 3) |
| | | | | | (Cytochrome c oxidase polypeptide VIII isoform |
| | | | | | 3)(COX VIII-3)(Cytochrome c oxidase subunit |
| | | | | | 8-3)(COX8-3)(Cytochrome c oxidase subunit |
| | | | | | VIIIC) |
| 352 | HLA-B*44:03_SENDIPSVAF | 283 | DC4L2 | ENSG00000176566 | DDB1-and CUL4-associated factor 4-like protein |
| | | | | | 2 (WD repeat-containing protein 21C) |
| 353 | HLA-A*33:01_NNFPHSIAR | 284 | KKLC1 | ENSG00000204019 | Kita-kyushu lung cancer antigen 1 (KK-LC-1) |
| | | | | | (Cancer/testis antigen 83) |
| 354 | HLA-A*31:01_RGNEVISVMNR | 285 | PPBN | ENSG00000163286 | Alkaline phosphatase, placental-like (EC 3.1.3.1) |
| | | | | | (ALP-1)(Alkaline phosphatase Nagao isozyme) |
| | | | | | (Germ cell alkaline phosphatase)(GCAP) |
| | | | | | (Placental alkaline phosphatase-like)(PLAP-like) |
| 355 | HLA-A*11:01_KTYETNLEIKK | 286 | NALP7 | ENSG00000167634 | NACHT, LRR and PYD domains-containing |
| | | | | | protein 7 (Nucleotide-binding oligomerization |
| | | | | | domain protein 12)(PYRIN-containing APAF1- |
| | | | | | like protein 3) |
| 356 | HLA-A*11:01_AALDNTNIGK | 287 | SMC1B | ENSG00000077935 | Structural maintenance of chromosomes protein |
| | | | | | 1B (SMC protein 1B)(SMC-1-beta)(SMC-1B) |
| 357 | HLA-A*29:02_ALASVGHLY | 288 | ONEC3 | ENSG00000205922 | One cut domain family member 3 (One cut |
| | | | | | homeobox 3)(Transcription factor ONECUT-3) |
| | | | | | (OC-3) |
| 358 | HLA-A*26:01_EVSNKIVGY | 289 | PERL | ENSG00000167419 | Lactoperoxidase (LPO)(EC 1.11.1.7)(Salivary |
| | | | | | peroxidase)(SPO) |
| 359 | HLA-A*29:02_SFLDASFVY | 290 | PERL | ENSG00000167419 | Lactoperoxidase (LPO)(EC 1.11.1.7)(Salivary |
| | | | | | peroxidase)(SPO) |
| 360 | HLA-A*11:01_TVSQAKVQVNK | 291 | PERL | ENSG00000167419 | Lactoperoxidase (LPO)(EC 1.11.1.7)(Salivary |
| | | | | | peroxidase)(SPO) |
| 361 | HLA-A*26:01_ELKQDISSF | 292 | TRPC5 | ENSG00000072315 | Short transient receptor potential channel 5 |
| | | | | | (TipC5)(Transient receptor protein 5)(TRP-5) |
| | | | | | (hTRP-5)(hTRP5) |
| 362 | HLA-B*18:01_VENEFKAEY | 293 | TRPC5 | ENSG00000072315 | Short transient receptor potential channel 5 |
| | | | | | (TipC5)(Transient receptor protein 5)(TRP-5) |
| | | | | | (hTRP-5)(hTRP5) |
| 363 | HLA-A*29:02_SFSNVWHLY | 294 | VRTN | ENSG00000133980 | Vertnin |
| 364 | HLA-A*03:01_ATAQPSQVRQK | 261 | FHL17 | ENSG00000132446 | Ferritin heavy polypeptide-like 17 (Cancer/testis |
| | | | | | antigen 38)(CT38) |
| 365 | HLA-B*51:01_DAAINSHI | 295 | FHL17 | ENSG00000132446 | Ferritin heavy polypeptide-like 17 (Cancer/testis |
| | | | | | antigen 38)(CT38) |
| 366 | HLA-A*29:02_VALENFFRY | 296 | FHL17 | ENSG00000132446 | Ferritin heavy polypeptide-like 17 (Cancer/testis |
| | | | | | antigen 38)(CT38) |
| 367 | HLA-B*44:03_AEMLTNVISRY | 297 | MAGC1 | ENSG00000155495 | Melanoma-associated antigen C1 (Cancer/testis |
| | | | | | antigen 7.1)(CT7.1)(MAGE-C1 antigen) |
| 368 | HLA-B*44:03_DEDGKIVGY | 164 | NAA11 | ENSG00000156269 | N-alpha-acetyltransferase 11 (EC 2.3.1.255)(N- |
| | | | | | terminal acetyltransferase complex ARD1 subunit |
| | | | | | homolog B)(hARD2)(NatA catalytic subunit |
| | | | | | Naa11) |
| 369 | HLA-A*11:01_KTLGKIAEK | 298 | SG1D1 | ENSG00000168515 | Secretoglobin family 1D member 1 (Lipophilin- |
| | | | | | A) |
| 370 | HLA-A*24:02_YYDLVESTF | 268 | TDT | ENSG00000107447 | DNA nucleotidylexotransferase (EC 2.7.7.31) |
| | | | | | (Terminal addition enzyme)(Terminal |
| | | | | | deoxynucleotidyltransfemse)(Terminal |
| | | | | | transferase) |
| 371 | HLA-A*30:02_AQAFTSGKY | 299 | TRI51 | ENSG00000124900 | Tripartite motif-containing protein 51 (SPRY |
| | | | | | domain-containing protein 5) |
| 372 | HLA-B*18:01_DEEDMQAVETY | 300 | PD L2 | ENSG00000163440 | Phosducin-like protein 2 |
| 373 | HLA-A*30:02_STKSVSTSY | 301 | sHT1F | ENSG00000179097 | 5-hydroxytryptamine receptor 1F (5-HT-1F)(5- |
| | | | | | HT1F)(Serotonin receptor 1F) |
| 374 | HLA-A*02:01_LVIDTVTEV | 302 | SPERT | ENSG00000174015 | Spermatid-associated protein (Protein chibby |
| | | | | | homolog 2) |
| 375 | HLA-C*05:01_VIDTVTEV | 303 | SPERT | ENSG00000174015 | Spermatid-associated protein (Protein chibby |
| | | | | | homolog 2) |
| 376 | HLA-B*51:01_YPLNRFSSV | 304 | SPERT | ENSG00000174015 | Spermatid-associated protein (Protein chibby |
| | | | | | homolog 2) |
| 377 | HLA-B*35:01_YPAERLTSF | 274 | CRBA4 | ENSG00000196431 | Beta-crystallin A4 (Beta-A4 crystallin) |
| 378 | HLA-B*18:01_TEIVLENNY | 305 | R4GMQ3 | ENSG00000107831 | Fibroblast growth factor 8 |
| 379 | HLA-A*01:01_YTALQNAKY | 306 | R4GMQ3 | ENSG00000107831 | Fibroblast growth factor 8 |
| 380 | HLA-A*33:01_DSFQKVILR | 307 | ZN728 | ENSG00000269067 | Zinc finger protein 728 |
| 381 | HLA-B*44:03_SEPQIVPITF | 308 | CNTP5 | ENSG00000155052 | Contactin-associated protein-like 5 (Cell |
| | | | | | recognition molecule Caspr5) |
| 382 | HLA-B*44:02_EEIIPLNRIY | 71 | TDRD1 | ENSG00000095627 | Tudor domain-containing protein 1 (Cancer/testis |
| | | | | | antigen 41.1)(CT41.1) |
| 383 | HLA-A*24:02_IYTGVTVSF | 309 | LMIP | ENSG00000105370 | Lens fiber membrane intrinsic protein (MP18) |
| | | | | | (MP19)(MP20) |
| 384 | HLA-A*29:02_SFAHQGLWRY | 310 | LMIP | ENSG00000105370 | Lens fiber membrane intrinsic protein (MP18) |
| | | | | | (MP19)(MP20) |
| 385 | HLA-B*44:03_EEIPQEIQRL | 311 | LRIQ4 | ENSG00000188306 | Leucine-rich repeat and IQ domain-containing |
| | | | | | protein 4 (Leucine-rich repeat-containing protein |
| | | | | | 64) |
| 386 | HLA-A*01:01_YIENNHLEY | 312 | LRIQ4 | ENSG00000188306 | Leucine-rich repeat and IQ domain-containing |
| | | | | | protein 4 (Leucine-rich repeat-containing protein |
| | | | | | 64) |
| 387 | HLA-A*01:01_HSEELDPQKY | 313 | TRPC5 | ENSG00000072315 | Short transient receptor potential channel 5 |
| | | | | | (TipC5)(Transient receptor protein 5)(TRP-5) |
| | | | | | (hTRP-5)(hTRP5) |
| 388 | HLA-B*44:02_AEMLESVIKNY | 24 | MAGA8 | ENSG00000156009 | Melanoma-associated antigen 8 (Cancer/testis |
| | | | | | antigen 1.8)(CT1.8)(MAGE-8 antigen) |
| 389 | HLA-B*44:03_AEMLESVIKNY | 24 | MAGA8 | ENSG00000156009 | Melanoma-associated antigen 8 (Cancer/testis |
| | | | | | antigen 1.8)(CT1.8)(MAGE-8 antigen) |
| 390 | HLA-C*02:02_AEMLESVIKNY | 24 | MAGA8 | ENSG00000156009 | Melanoma-associated antigen 8 (Cancer/testis |
| | | | | | antigen 1.8)(CT1.8)(MAGE-8 antigen) |
| 391 | HLA-B*44:03_AETSYVKVL | 10 | MAGA8 | ENSG00000156009 | Melanoma-associated antigen 8 (Cancer/testis |
| | | | | | antigen 1.8)(CT1.8)(MAGE-8 antigen) |
| 392 | HLA-A*02:01_ALDEKVAEL | 314 | MAGA8 | ENSG00000156009 | Melanoma-associated antigen 8 (Cancer/testis |
| | | | | | antigen 1.8)(CT1.8)(MAGE-8 antigen) |
| 393 | HLA-A*02:07_ALDEKVAEL | 314 | MAGA8 | ENSG00000156009 | Melanoma-associated antigen 8 (Cancer/testis |
| | | | | | antigen 1.8)(CT1.8)(MAGE-8 antigen) |
| 394 | HLA-A*02:01_GLYDGREHSV | 315 | MAGA8 | ENSG00000156009 | Melanoma-associated antigen 8 (Cancer/testis |
| | | | | | antigen 1.8)(CT1.8)(MAGE-8 antigen) |
| 395 | HLA-B*44:03_SESSTILVVRY | 316 | SPNXB | ENSG00000227234 | Sperm protein associated with the nucleus on the |
| | | | | | X chromosome B1 (Cancer/testis antigen 11.2) |
| | | | | | (CT11.2)(Nuclear-associated protein SPAN-Xb) |
| | | | | | (SPANX-B)(SPANX family member B1) |
| | | | | | (SPANX family member Fl) |
| 396 | HLA-A*01:01_QTEFPTTYY | 317 | GFY | ENSG00000261949 | Golgi-associated olfactory signaling regulator |
| | | | | | (Protein Goofy) |
| 397 | HLA-B*18:01_TEFPTTYY | 318 | GFY | ENSG00000261949 | Golgi-associated olfactory signaling regulator |
| | | | | | (Protein Goofy) |
| 398 | HLA-A*01:01_TSDPQISTSLY | 319 | GFY | ENSG00000261949 | Golgi-associated olfactory signaling regulator |
| | | | | | (Protein Goofy) |
| 399 | HLA-A*02:07_VLDEEVSNL | 320 | SUN3 | ENSG00000164744 | SUN domain-containing protein 3 (Sad1/unc-84 |
| | | | | | domain-containing protein 1) |
| 400 | HLA-B*46:01_FLITQATAY | 321 | NBPF4 | ENSG00000196427 | Neuroblastoma breakpoint family member 4 |
| 401 | HLA-A*30:02_ALQGALGLY | 322 | PPAT | ENSG00000142513 | Testicular acid phosphatase (EC 3.1.3.2) |
| 402 | HLA-B*51:01_YPMDPHKEV | 323 | PPAT | ENSG00000142513 | Testicular acid phosphatase (EC 3.1.3.2) |
| 403 | HLA-B*51:01_LAFLVGQSI | 324 | PIWL1 | ENSG00000125207 | Piwi-like protein 1 (EC 3.1.26.-) |
| 404 | HLA-B*35:01_MPSEVSEVL | 325 | E9PRF5 | ENSG00000233436 | BTB/POZ domain-containing protein 18 |
| | | | | | (Fragment) |
| 405 | HLA-A*02:07_KLFTSVFGV | 267 | TDT | ENSG00000107447 | DNA nucleotidylexotransferase (EC 2.7.7.31) |
| | | | | | (Terminal addition enzyme)(Terminal |
| | | | | | deoxynucleotidyltransfemse)(Terminal |
| | | | | | transferase) |
| 406 | HLA-B*35:01_TAAAFTISY | 326 | S22AD | ENSG00000172940 | Solute carrier family 22 member 13 (Organic |
| | | | | | cation transporter-lace 3)(ORCTL-3) |
| 407 | HLA-A*29:02_FFLHPISFY | 327 | MBOA4 | ENSG00000177669 | Ghrelin O-acyltransferase (EC 2.3.1.-) |
| | | | | | (Membrane-bound O-acyltransferase domain- |
| | | | | | containing protein 4)(O-acyltransferase domain- |
| | | | | | containing protein 4) |
| 408 | HLA-A*11:01_ASQISSETLIK | 328 | DPPA3 | ENSG00000187569 | Developmental pluripotency-associated protein 3 |
| | | | | | (Stella-related protein) |
| 409 | HLA-A*11:01_ASALFQSNK | 329 | RNS10 | ENSG00000182545 | Inactive ribonuclease-like protein 10 |
| 410 | HLA-B*18:01_SEESVLVGY | 330 | TDRD1 | ENSG00000095627 | Tudor domain-containing protein 1 (Cancer/testis |
| | | | | | antigen 41.1)(CT41.1) |
| 411 | HLA-A*26:01_ETSYVKVLEY | 331 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 412 | HLA-B*18:01_DEGISSLF | 332 | KCNH5 | ENSG00000140015 | Potassium voltage-gated channel subfamily H |
| | | | | | member 5 (Ether-a-go-go potassium channel 2) |
| | | | | | (hEAG2)(Voltage-gated potassium channel |
| | | | | | subunit Kv10.2) |
| 413 | HLA-A*03:01_KVNSPIRMK | 333 | KCNH5 | ENSG00000140015 | Potassium voltage-gated channel subfamily H |
| | | | | | member 5 (Ether-a-go-go potassium channel 2) |
| | | | | | (hEAG2)(Voltage-gated potassium channel |
| | | | | | subunit Kv10.2) |
| 414 | HLA-B*35:01_LPYDIINAF | 334 | KCNH5 | ENSG00000140015 | Potassium voltage-gated channel subfamily H |
| | | | | | member 5 (Ether-a-go-go potassium channel 2) |
| | | | | | (hEAG2)(Voltage-gated potassium channel |
| | | | | | subunit Kv10.2) |
| 415 | HLA-C*05:01_NVDEGISSL | 335 | KCNH5 | ENSG00000140015 | Potassium voltage-gated channel subfamily H |
| | | | | | member 5 (Ether-a-go-go potassium channel 2) |
| | | | | | (hEAG2)(Voltage-gated potassium channel |
| | | | | | subunit Kv10.2) |
| 416 | HLA-A*11:01_SVLQQLTPMNK | 336 | KCNH5 | ENSG00000140015 | Potassium voltage-gated channel subfamily H |
| | | | | | member 5 (Ether-a-go-go potassium channel 2) |
| | | | | | (hEAG2)(Voltage-gated potassium channel |
| | | | | | subunit Kv10.2) |
| 417 | HLA-B*35:01_TPIQTSLAY | 337 | KCNH5 | ENSG00000140015 | Potassium voltage-gated channel subfamily H |
| | | | | | member 5 (Ether-a-go-go potassium channel 2) |
| | | | | | (hEAG2)(Voltage-gated potassium channel |
| | | | | | subunit Kv10.2) |
| 418 | HLA-B*35:01_EALTPHSSY | 338 | I22R2 | ENSG00000164485 | Interleukin-22 receptor subunit alpha-2 (IL-22 |
| | | | | | receptor subunit alpha-2)(IL-22R-alpha-2)(IL- |
| | | | | | 22RA2)(Cytokine receptor class-II member 10) |
| | | | | | (Cytokine receptor family 2 member 10)(CRF2- |
| | | | | | 10)(Cytokine receptor family type 2, soluble 1) |
| | | | | | (CRF2-S1)(Interleukin-22-binding protein)(IL- |
| | | | | | 22BP)(IL22BP)(ZcytoR16) |
| 419 | HLA-A*01:01_FTEIVLENNY | 339 | R4GMQ3 | ENSG00000107831 | Fibroblast growth factor 8 |
| 420 | HLA-B*44:03_AEVPIEPHW | 340 | GCNT7 | ENSG00000124091 | Beta-1,3-galactosyl-O-glycosyl-glycoprotein |
| | | | | | beta-1,6-N-acetylglucosaminyltransferase 7 (EC |
| | | | | | 2.4.1.-) |
| 421 | HLA-B*35:01_LPFTIISM | 341 | TDT | ENSG00000107447 | DNA nucleotidylexotransferase (EC 2.7.7.31) |
| | | | | | (Terminal addition enzyme)(Teiminal |
| | | | | | deoxynucleotidyltransferase)(Terminal |
| | | | | | transferase) |
| 422 | HLA-B*35:01_DPSQFNPTY | 342 | DPPA3 | ENSG00000187569 | Developmental pluripotency-associated protein 3 |
| | | | | | (Stella-related protein) |
| 423 | HLA-B*44:03_AEILKNEAY | 343 | A0A1BOGTN1 | ENSG00000224960 | Putative SMEK homolog 3 |
| 424 | HLA-C*02:02_AEILKNEAY | 343 | A0A1BOGTN1 | ENSG00000224960 | Putative SMEK homolog 3 |
| 425 | HLA-B*44:02_AENQGLVLKF | 344 | A0A1BOGTN1 | ENSG00000224960 | Putative SMEK homolog 3 |
| 426 | HLA-B*44:03_AENQGLVLKF | 344 | A0A1BOGTN1 | ENSG00000224960 | Putative SMEK homolog 3 |
| 427 | HLA-A*02:01_ALFETLIQL | 345 | A0A1BOGTN1 | ENSG00000224960 | Putative SMEK homolog 3 |
| 428 | HLA-A*26:01_ESIEYVQTF | 346 | A0A1BOGTN1 | ENSG00000224960 | Putative SMEK homolog 3 |
| 429 | HLA-A*26:01_EVIPITNSEL | 347 | A0A1BOGTN1 | ENSG00000224960 | Putative SMEK homolog 3 |
| 430 | HLA-B*51:01_LPALKIVMI | 348 | A0A1BOGTN1 | ENSG00000224960 | Putative SMEK homolog 3 |
| 431 | HLA-B*18:01_NEMSVISNM | 349 | A0A1BOGTN1 | ENSG00000224960 | Putative SMEK homolog 3 |
| 432 | HLA-A*01:01_NTEGLHHLY | 350 | A0A1BOGTN1 | ENSG00000224960 | Putative SMEK homolog 3 |
| 433 | HLA-A*24:02_NYIIKGNLF | 351 | A0A1BOGTN1 | ENSG00000224960 | Putative SMEK homolog 3 |
| 434 | HLA-A*02:01_QIADIVTSV | 352 | A0A1BOGTN1 | ENSG00000224960 | Putative SMEK homolog 3 |
| 435 | HLA-C*01:02_VIPITNSEL | 353 | A0A1B0GTN1 | ENSG00000224960 | Putative SMEK homolog 3 |
| 436 | HLA-B*18:01_SENDIPSVAF | 283 | DC4L2 | ENSG00000176566 | DDB1-and CUL4-associated factor 4-like protein |
| | | | | | 2 (WD repeat-containing protein 21C) |
| 437 | HLA-C*01:02_AAPAGPGEL | 354 | FOXI3 | ENSG00000214336 | Forkhead box protein I3 |
| 438 | HLA-C*02:02_AESSSKRSF | 355 | TRPC5 | ENSG00000072315 | Short transient receptor potential channel 5 |
| | | | | | (TipC5)(Transient receptor protein 5)(TRP-5) |
| | | | | | (hTRP-5)(hTRP5) |
| 439 | HLA-B*18:01_TEFVGATMF | 356 | TRPC5 | ENSG00000072315 | Short transient receptor potential channel 5 |
| | | | | | (TipC5)(Transient receptor protein 5)(TRP-5) |
| | | | | | (hTRP-5)(hTRP5) |
| 440 | HLA-A*29:02_VYVGDALLY | 357 | TRPC5 | ENSG00000072315 | Short transient receptor potential channel 5 |
| | | | | | (TrpC5)(Transient receptor protein 5)(TRP-5) |
| | | | | | (hTRP-5)(hTRP5) |
| 441 | HLA-B*51:01_HPLNGQPLI | 358 | LUZP4 | ENSG00000102021 | Leucine zipper protein 4 (Cancer/testis antigen |
| | | | | | 28)(CT-28)(CT28)(Tumor antigen HOM-TES- |
| | | | | | 85) |
| 442 | HLA-A*02:07_LLDGFMITL | 359 | PASD1 | ENSG00000166049 | Circadian clock protein PASD1 (Cancer/testis |
| | | | | | antigen 63)(CT63)(OX-TES-1)(PAS domain- |
| | | | | | containing protein 1) |
| 443 | HLA-A*24:02_VYQKIILKF | 360 | PASD1 | ENSG00000166049 | Circadian clock protein PASD1 (Cancer/testis |
| | | | | | antigen 63)(CT63)(OX-TES-1)(PAS domain- |
| | | | | | containing protein 1) |
| 444 | HLA-B*35:01_EPLSVTASY | 361 | VCX3 | ENSG00000169059 | Variable charge X-linked protein 3 (Variable |
| | | | | | charge protein on X with eight repeats)(VCX-8r) |
| | | | | | (Variably charged protein X-A)(VCX-A) |
| 445 | HLA-B*51:01_DAFVPFSI | 362 | CP2AD | ENSG00000197838 | Cytochrome P450 2A13 (EC 1.14.14.1) |
| | | | | | (CYPIIA13) |
| 446 | HLA-A*26:01_ETVSTTLRY | 363 | CP2AD | ENSG00000197838 | Cytochrome P450 2A13 (EC 1.14.14.1) |
| | | | | | (CYPIIA13) |
| 447 | HLA-A*01:01_GTETVSTTLRY | 364 | CP2AD | ENSG00000197838 | Cytochrome P450 2A13 (EC 1.14.14.1) |
| | | | | | (CYPIIA13) |
| 448 | HLA-A*02:01_FLWGPRALVET | 41 | MAGAC | ENSG00000213401 | Melanoma-associated antigen 12 (Cancer/testis |
| | | | | | antigen 1.12)(CT1.12)(MAGE-12 antigen) |
| | | | | | (MAGE12F antigen) |
| 449 | HLA-A*11:01_AVNPELAPVMK | 365 | SPT22 | ENSG00000141255 | Spermatogenesis-associated protein 22 (Testis |
| | | | | | development protein NYD-SP20) |
| 450 | HLA-C*16:01_SAVTPGPYY | 366 | SPT22 | ENSG00000141255 | Spermatogenesis-associated protein 22 (Testis |
| | | | | | development protein NYD-SP20) |
| 451 | HLA-A*31:01_RVQVWFQNR | 367 | ALX3 | ENSG00000156150 | Homeobox protein aristaless-like 3 (Proline-rich |
| | | | | | transcription factor ALX3) |
| 452 | HLA-A*02:07_KMAELVHFL | 368 | MAGAC | ENSG00000213401 | Melanoma-associated antigen 12 (Cancer/testis |
| | | | | | antigen 1.12)(CT1.12)(MAGE-12 antigen) |
| | | | | | (MAGE12F antigen) |
| 453 | HLA-A*29:02_IFINKEDSLLY | 369 | C295L | ENSG00000178404 | CEP295 N-terminal-like protein (KIAA1731 N- |
| | | | | | terminal like protein) |
| 454 | HLA-A*02:01_GLWEIENNPTV | 370 | HDGL1 | ENSG00000112273 | Hepatoma-derived growth factor-like protein 1 |
| | | | | | (PWWP domain-containing protein 1) |
| 455 | HLA-A*03:01_SAYGMPMYK | 371 | HDGL1 | ENSG00000112273 | Hepatoma-derived growth factor-like protein 1 |
| | | | | | (PWWP domain-containing protein 1) |
| 456 | HLA-A*11:01_SAYGMPMYK | 371 | HDGL1 | ENSG00000112273 | Hepatoma-derived growth factor-like protein 1 |
| | | | | | (PWWP domain-containing protein 1) |
| 457 | HLA-B*35:01_AAAAAAATY | 372 | NKX24 | ENSG00000125816 | Homeobox protein Nkx-2.4 (Homeobox protein |
| | | | | | NK-2 homolog D) |
| 458 | HLA-A*03:01_RVAVPVLVK | 373 | NKX24 | ENSG00000125816 | Homeobox protein Nkx-2.4 (Homeobox protein |
| | | | | | NK-2 homolog D) |
| 459 | HLA-A*30:02_SQFPHGAMGSY | 374 | NKX24 | ENSG00000125816 | Homeobox protein Nkx-2.4 (Homeobox protein |
| | | | | | NK-2 homolog D) |
| 460 | HLA-B*51:01_DAVAAMSV | 375 | RBM46 | ENSG00000151962 | Probable RNA-binding protein 46 (Cancer/testis |
| | | | | | antigen 68)(CT68)(RNA-binding motif protein |
| | | | | | 46) |
| 461 | HLA-A*29:02_NWAPPEYYLY | 376 | RBM46 | ENSG00000151962 | Probable RNA-binding protein 46 (Cancer/testis |
| | | | | | antigen 68)(CT68)(RNA-binding motif protein |
| | | | | | 46) |
| 462 | HLA-B*18:01_TEETIKAEF | 377 | RBM46 | ENSG00000151962 | Probable RNA-binding protein 46 (Cancer/testis |
| | | | | | antigen 68)(CT68)(RNA-binding motif protein |
| | | | | | 46) |
| 463 | HLA-A*29:02_WAPPEYYLY | 378 | RBM46 | ENSG00000151962 | Probable RNA-binding protein 46 (Cancer/testis |
| | | | | | antigen 68)(CT68)(RNA-binding motif protein |
| | | | | | 46) |
| 464 | HLA-C*02:02_KEVDPTGHSF | 379 | MAGAA | ENSG00000124260 | Melanoma-associated antigen 10 (Cancer/testis |
| | | | | | antigen 1.10)(CT1.10)(MAGE-10 antigen) |
| 465 | HLA-B*18:01_DEEQNLVAF | 380 | PRDM7 | ENSG00000126856 | Probable histone-lysine N-methyltransferase |
| | | | | | PRDM7 (EC 2.1.1.43)(PR domain zinc finger |
| | | | | | protein 7)(PR domain-containing protein 7) |
| 466 | HLA-B*44:03_EEAANSGYSW | 381 | PRDM7 | ENSG00000126856 | Probable histone-lysine N-methyltransferase |
| | | | | | PRDM7 (EC 2.1.1.43)(PR domain zinc finger |
| | | | | | protein 7)(PR domain-containing protein 7) |
| 467 | HLA-C*02:02_EEAANSGYSW | 381 | PRDM7 | ENSG00000126856 | Probable histone-lysine N-methyltransferase |
| | | | | | PRDM7 (EC 2.1.1.43)(PR domain zinc finger |
| | | | | | protein 7)(PR domain-containing protein 7) |
| 468 | HLA-B*44:03_EEQNLVAFQY | 382 | PRDM7 | ENSG00000126856 | Probable histone-lysine N-methyltransferase |
| | | | | | PRDM7 (EC 2.1.1.43)(PR domain zinc finger |
| | | | | | protein 7)(PR domain-containing protein 7) |
| 469 | HLA-A*11:01_STEDEEQLLQK | 383 | TDT | ENSG00000107447 | DNA nucleotidylexotransferase (EC 2.7.7.31) |
| | | | | | (Terminal addition enzyme)(Teiminal |
| | | | | | deoxynucleotidyltransfemse)(Terminal |
| | | | | | transferase) |
| 470 | HLA-C*16:01_ASIDREIAM | 384 | PRD13 | ENSG00000112238 | PR domain zinc finger protein 13 (EC 2.1.1.-) |
| | | | | | (PR domain-containing protein 13) |
| 471 | HLA-A*03:01_RLGPVPGTFK | 385 | PRD13 | ENSG00000112238 | PR domain zinc finger protein 13 (EC 2.1.1.-) |
| | | | | | (PR domain-containing protein 13) |
| 472 | HLA-A*11:01_SSSQTAAQVTK | 386 | BRDT | ENSG00000137948 | Brodomain testis-specific protein |
| | | | | | (Cancer/testis antigen 9)(CT9)(RING3-like |
| | | | | | protein) |
| 473 | HLA-B*44:02_AEMAVGLVVF | 282 | COX8C | ENSG00000187581 | Cytochrome c oxidase subunit 8C, mitochondrial |
| | | | | | (Cytochrome c oxidase polypeptide 8 isoform 3) |
| | | | | | (Cytochrome c oxidase polypeptide VIII isoform |
| | | | | | 3)(COX VIII-3)(Cytochrome c oxidase subunit |
| | | | | | 8-3)(COX8-3)(Cytochrome c oxidase subunit |
| | | | | | VIIIC) |
| 474 | HLA-A*02:07_FLDLQVNSL | 387 | TERT | ENSG00000164362 | Telomerase reverse transcriptase (EC 2.7.7.49) |
| | | | | | (HEST2)(Telomerase catalytic subunit) |
| | | | | | (Telomerase-associated protein 2)(TP2) |
| 475 | HLA-A*29:02_VLREIEDEWLY | 388 | DPPA3 | ENSG00000187569 | Developmental pluripotency-associated protein 3 |
| | | | | | (Stella-related protein) |
| 476 | HLA-B*18:01_DESITFHSI | 389 | 1A1L2 | ENSG00000205126 | Probable inactive 1-aminocyclopropane-1- |
| | | | | | carboxylate synthase-like protein 2 (ACC |
| | | | | | synthase-like protein 2) |
| 477 | HLA-B*18:01_EEVARFLTY | 390 | 1A1L2 | ENSG00000205126 | Probable inactive 1-aminocyclopropane-1- |
| | | | | | carboxylate synthase-like protein 2 (ACC |
| | | | | | synthase-like protein 2) |
| 478 | HLA-B*44:03_EEVARFLTY | 390 | 1A1L2 | ENSG00000205126 | Probable inactive 1-aminocyclopropane-1- |
| | | | | | carboxylate synthase-like protein 2 (ACC |
| | | | | | synthase-like protein 2) |
| 479 | HLA-A*26:01_EVASAVSAF | 391 | 1A1L2 | ENSG00000205126 | Probable inactive 1-aminocyclopropane-1- |
| | | | | | carboxylate synthase-like protein 2 (ACC |
| | | | | | synthase-like protein 2) |
| 480 | HLA-A*02:07_LLPGSIHFV | 392 | PTX4 | ENSG00000251692 | Pentraxin-4 |
| 481 | HLA-B*44:03_EEQLLQKVM | 393 | TDT | ENSG00000107447 | DNA nucleotidylexotransferase (EC 2.7.7.31) |
| | | | | | (Terminal addition enzyme)(Teiminal |
| | | | | | deoxynucleotidyltransfemse)(Terminal |
| | | | | | transferase) |
| 482 | HLA-A*02:07_KVLEFLAKV | 394 | MAGB2 | ENSG00000099399 | Melanoma-associated antigen B2 (Cancer/testis |
| | | | | | antigen 3.2)(CT3.2)(DSS-AHC critical interval |
| | | | | | MAGE supeifamily 6)(DAM6)(MAGE XP-2 |
| | | | | | antigen)(MAGE-B2 antigen) |
| 483 | HLA-A*02:07_SLDDIIIYKEL | 395 | LUZP4 | ENSG00000102021 | Leucine zipper protein 4 (Cancer/testis antigen |
| | | | | | 28)(CT-28)(CT28)(Tumor antigen HOM-TES- |
| | | | | | 85) |
| 484 | HLA-B*44:03_KEGEAVEFTF | 396 | LN28A | ENSG00000131914 | Protein lin-28 homolog A (Lin-28A)(Zinc finger |
| | | | | | CCHC domain-containing protein 1) |
| 485 | HLA-B*18:01_DEGAMLLY | 397 | NALP7 | ENSG00000167634 | NACHT, LRR and PYD domains-containing |
| | | | | | protein 7 (Nucleotide-binding oligomerization |
| | | | | | domain protein 12)(PYRIN-containing APAF1- |
| | | | | | like protein 3) |
| 486 | HLA-A*11:01_KTYETNLEIK | 398 | NALP7 | ENSG00000167634 | NACHT, LRR and PYD domains-containing |
| | | | | | protein 7 (Nucleotide-binding oligomerization |
| | | | | | domain protein 12)(PYRIN-containing APAF1- |
| | | | | | like protein 3) |
| 487 | HLA-A*01:01_LLDEGAMLLY | 399 | NALP7 | ENSG00000167634 | NACHT, LRR and PYD domains-containing |
| | | | | | protein 7 (Nucleotide-binding oligomerization |
| | | | | | domain protein 12)(PYRIN-containing APAF1- |
| | | | | | like protein 3) |
| 488 | HLA-B*35:01_TPLVIAISY | 400 | NPBW1 | ENSG00000183729 | Neuropeptides B/W receptor type 1(G-protein |
| | | | | | coupled receptor 7) |
| 489 | HLA-B*44:03_VEVGEVKSW | 401 | RFPLB | ENSG00000251258 | Ret finger protein-like 4B (RING finger protein |
| | | | | | 211) |
| 490 | HLA-C*02:02_VEVGEVKSW | 401 | RFPLB | ENSG00000251258 | Ret finger protein-like 4B (RING finger protein |
| | | | | | 211) |
| 491 | HLA-B*44:03_WEVEVGEVKSW | 402 | RFPLB | ENSG00000251258 | Ret finger protein-like 4B (RING finger protein |
| | | | | | 211) |
| 492 | HLA-A*26:01_TTAPGTVHSY | 403 | Q5JUY5 | ENSG00000117400 | Thrombopoietin receptor |
| 493 | HLA-C*16:01_ASSQVPRVM | 404 | PABP3 | ENSG00000151846 | Polyadenylate-binding protein 3 (PABP-3) |
| | | | | | (Poly(A)-binding protein 3)(Testis-specific |
| | | | | | poly(A)-binding protein) |
| 494 | HLA-A*01:01_WSDSSVTTY | 405 | CD051 | ENSG00000237136 | Uncharacterized protein C4orf51 |
| 495 | HLA-C*02:02_KAFDDIATY | 406 | SSX1 | ENSG00000126752 | Protein SSX1 (Cancer/testis antigen 5.1)(CT5.1) |
| | | | | | (Synovial sarcoma, X breakpoint 1) |
| 496 | HLA-A*29:02_HVSNLVFAY | 407 | CTSRD | ENSG00000174898 | Cation channel sperm-associated protein subunit |
| | | | | | delta (CatSper-delta)(CatSperdelta) |
| | | | | | (Transmembrane protein 146) |
| 497 | HLA-A29:02_ILGSVWLAY | 408 | CTSRD | ENSG00000174898 | Cation channel sperm-associated protein subunit |
| | | | | | delta (CatSper-delta)(CatSperdelta) |
| | | | | | (Transmembrane protein 146) |
| 498 | HLA-B*44:03_KEFGGPFFW | 409 | CTSRD | ENSG00000174898 | Cation channel sperm-associated protein subunit |
| | | | | | delta (CatSper-delta)(CatSperdelta) |
| | | | | | (Transmembrane protein 146) |
| 499 | HLA-B*35:01_LPFTIPTSM | 410 | CTSRD | ENSG00000174898 | Cation channel sperm-associated protein subunit |
| | | | | | delta (CatSper-delta)(CatSperdelta) |
| | | | | | (Transmembrane protein 146) |
| 500 | HLA-A*11:01_VVNQGKGMFK | 411 | CTSRD | ENSG00000174898 | Cation channel sperm-associated protein subunit |
| | | | | | delta (CatSper-delta)(CatSperdelta) |
| | | | | | (Transmembrane protein 146) |
| 501 | HLA-A*24:02_VYGAFPVQL | 412 | CTSRD | ENSG00000174898 | Cation channel sperm-associated protein subunit |
| | | | | | delta (CatSper-delta)(CatSperdelta) |
| | | | | | (Transmembrane protein 146) |
| 502 | HLA-A*01:01_YTSDGNTKY | 413 | CTSRD | ENSG00000174898 | Cation channel sperm-associated protein subunit |
| | | | | | delta (CatSper-delta)(CatSperdelta) |
| | | | | | (Transmembrane protein 146) |
| 503 | HLA-A*24:02_VYGSYLYKL | 414 | DPPA5 | ENSG00000203909 | Developmental pluripotency-associated 5 protein |
| | | | | | (hDPPA5)(Embtyonal stem cell-specific gene 1 |
| | | | | | protein)(ESG-1) |
| 504 | HLA-A*30:02_QISEVEPKY | 415 | NAA11 | ENSG00000156269 | N-alpha-acetyltransferase 11 (EC 2.3.1.255)(N- |
| | | | | | tenninal acetyltransferase complex ARD1 subunit |
| | | | | | homolog B)(hARD2)(NatA catalytic subunit |
| | | | | | Naa11) |
| 505 | HLA-A*29:02_HAPNLPYRY | 416 | I22R2 | ENSG00000164485 | Interleukin-22 receptor subunit alpha-2 (IL-22 |
| | | | | | receptor subunit alpha-2)(IL-22R-alpha-2)(IL- |
| | | | | | 22RA2)(Cytokine receptor class-II member 10) |
| | | | | | (Cytokine receptor family 2 member 10)(CRF2- |
| | | | | | 10)(Cytokine receptor family type 2, soluble 1) |
| | | | | | (CRF2-S1)(Interleukin-22-binding protein)(IL- |
| | | | | | 22BP)(IL22BP)(ZcytoR16) |
| 506 | HLA-B*35:01_LPFDGSPKITY | 417 | ERVV2 | ENSG00000268964 | Endogenous retrovirus group V member 2 Env |
| | | | | | polyprotein (HERV-V_19q13.41 provirus |
| | | | | | ancestral Env polyprotein 2) |
| 507 | HLA-B*51:01_LPFDGSPKI | 418 | ERVV2 | ENSG00000268964 | Endogenous retrovirus group V member 2 Env |
| | | | | | polyprotein (HERV-V_19q13.41 provirus |
| | | | | | ancestral Env polyprotein 2) |
| 508 | HLA-A*11:01_SVIGGPSTYK | 419 | ERVV2 | ENSG00000268964 | Endogenous retrovirus group V member 2 Env |
| | | | | | polyprotein (HERV-V_19q13.41 provirus |
| | | | | | ancestral Env polyprotein 2) |
| 509 | HLA-A*03:01_TIYNTTQPRQK | 420 | ERVV2 | ENSG00000268964 | Endogenous retrovirus group V member 2 Env |
| | | | | | polyprotein (HERV-V_19q13.41 provirus |
| | | | | | ancestral Env polyprotein 2) |
| 510 | HLA-B*35:01_EAFLSPEY | 421 | PPAT | ENSG00000142513 | Testicular acid phosphatase (EC 3.1.3.2) |
| 511 | HLA-C*16:01_AAMNIARAL | 422 | RNF17 | ENSG00000132972 | RING finger protein 17 (Tudor domain-containing protein 4) |
| 512 | HLA-A*11:01_ASYEIGYILK | 423 | RNF17 | ENSG00000132972 | RING finger protein 17 (Tudor domain-containing protein 4) |
| 513 | HLA-A*26:01_EVVGGAVRVQY | 424 | RNF17 | ENSG00000132972 | RING finger protein 17 (Tudor domain-containing protein 4) |
| 514 | HLA-B*46:01_LVKEGLASY | 425 | RNF17 | ENSG00000132972 | RING finger protein 17 (Tudor domain-containing protein 4) |
| 515 | HLA-A*02:01_ALYDGLTLV | 426 | VRTN | ENSG00000133980 | Vertnin |
| 516 | HLA-A*02:01_SLLKLIVEL | 427 | TRIMM | ENSG00000179046 | Probable E3 ubiquitin-protein ligase TRIML2 |
| | | | | | (EC 2.3.2.27)(RING-type E3 ubiquitin |
| | | | | | transferase TRIML2)(SPRY domain-containing |
| | | | | | protein 6)(Tripartite motif family-like protein 2) |
| 517 | HLA-B*35:01_IASNYNVSY | 428 | PTX4 | ENSG00000251692 | Pentraxin-4 |
| 518 | HLA-A*30:02_RQAPGSDPVRY | 429 | MAGA8 | ENSG00000156009 | Melanoma-associated antigen 8 (Cancer/testis |
| | | | | | antigen 1.8)(CT1.8)(MAGE-8 antigen) |
| 519 | HLA-B*44:02_AEMLTNVISRY | 297 | MAGC1 | ENSG00000155495 | Melanoma-associated antigen C1 (Cancer/testis |
| | | | | | antigen 7.1)(CT7.1)(MAGE-C1 antigen) |
| 520 | HLA-B*46:01_STKSVSTSY | 301 | 5HT1F | ENSG00000179097 | 5-hydroxyttyptamine receptor 1F (5-HT-1F)(5- |
| | | | | | HT1F)(Serotonin receptor 1F) |
| 521 | HLA-B*35:01_MAATGVSSM | 430 | SAGE1 | ENSG00000181433 | Sarcoma antigen 1 (Cancer/testis antigen 14) |
| | | | | | (CT14) |
| 522 | HLA-A*33:01_DTSPLLLGR | 431 | TIFAB | ENSG00000255833 | TRAF-interacting protein with FHA domain- |
| | | | | | containing protein B (TWA-like protein) |
| 523 | HLA-A*29:02_HVSPSPLIY | 432 | TIFAB | ENSG00000255833 | TRAF-interacting protein with FHA domain- |
| | | | | | containing protein B (TWA-like protein) |
| 524 | HLA-B*35:01_HVSPSPLIY | 432 | TIFAB | ENSG00000255833 | TRAF-interacting protein with FHA domain- |
| | | | | | containing protein B (TWA-like protein) |
| 525 | HLA-A*29:02_WVNGLTLRY | 433 | TIFAB | ENSG00000255833 | TRAF-interacting protein with FHA domain- |
| | | | | | containing protein B (TWA-like protein) |
| 526 | HLA-A*11:01_GTISFVQYK | 434 | J3KR52 | ENSG00000185055 | EF-hand calcium-binding domain-containing |
| | | | | | protein 10 |
| 527 | HLA-C*04:01_AFVTMTGGF | 435 | TDT | ENSG00000107447 | DNA nucleotidylexotransferase (EC 2.7.7.31) |
| | | | | | (Terminal addition enzyme)(Terminal |
| | | | | | deoxynucleotidyltransfemse)(Terminal |
| | | | | | transferase) |
| 528 | HLA-A*29:02_NLWEKKGLLLY | 436 | TDT | ENSG00000107447 | DNA nucleotidylexotransferase (EC 2.7.7.31) |
| | | | | | (Terminal addition enzyme)(Terminal |
| | | | | | deoxynucleotidyltransfemse)(Terminal |
| | | | | | transferase) |
| 529 | HLA-A*26:01_EVISQLTRV | 437 | SYCY1 | ENSG00000242950 | Syncytin-1 (Endogenous retrovirus group W |
| | | | | | member 1)(Env-W)(Envelope polyprotein |
| | | | | | gPr73)(Enverin)(HERV-7q Envelope protein) |
| | | | | | (HERV-W envelope protein)(HERV-W_7q21.2 |
| | | | | | provirus ancestral Env polyprotein)(Syneytin) |
| | | | | | [Cleaved into: Surface protein (SU)(gp50); |
| | | | | | Transmembrane protein (TM)(gp24)] |
| 530 | HLA-A*01:01_YTEQDLYSY | 438 | SYCY1 | ENSG00000242950 | Syneytin-1 (Endogenous retrovirus group W |
| | | | | | member 1)(Env-W)(Envelope polyprotein |
| | | | | | gPr73)(Enverin)(HERV-7q Envelope protein) |
| | | | | | (HERV-W envelope protein)(HERV-W_7q21.2 |
| | | | | | provirus ancestral Env polyprotein)(Syncytin) |
| | | | | | [Cleaved into: Surface protein (SU)(gp50); |
| | | | | | Transmembrane protein (TM)(gp24)] |
| 531 | HLA-A*24:02_NYFLDPVTI | 439 | TRI51 | ENSG00000124900 | Tripartite motif-containing protein 51 (SPRY |
| | | | | | domain-containing protein 5) |
| 532 | HLA-A*02:07_AVDEESPFL | 440 | HDGL1 | ENSG00000112273 | Hepatoma-derived growth factor-like protein 1 |
| | | | | | (PWWP domain-containing protein 1) |
| 533 | HLA-B*51:01_TPYHLSTVV | 441 | NPBW1 | ENSG00000183729 | Neuropeptides B/W receptor type 1(G-protein |
| | | | | | coupled receptor 7) |
| 534 | HLA-A*01:01_NLDHYTNAY | 442 | GLYL3 | ENSG00000203972 | Glycine N-acyltransferase-like protein 3 (EC |
| | | | | | 2.3.1.-) |
| 535 | HLA-B*51:01_DAFDVASFL | 443 | STRA8 | ENSG00000146857 | Stimulated by retinoic acid gene 8 protein |
| | | | | | homolog |
| 536 | HLA-A*29:02_RTFPITGLRY | 444 | DYTN | ENSG00000232125 | Dystrotelin |
| 537 | HLA-A*01:01_FTEEDLHFVLY | 445 | PRD14 | ENSG00000147596 | PR domain zinc finger protein 14 (EC 2.1.1.-) |
| | | | | | (PR domain-containing protein 14) |
| 538 | HLA-A*29:02_IFVSPKGVLAY | 446 | S7A13 | ENSG00000164893 | Solute carrier family 7 member 13 (Sodium- |
| | | | | | independent aspartate/glutamate transporter 1) |
| | | | | | (X-amino acid transporter 2) |
| 539 | HLA-B*44:03_QEGQLPLLF | 447 | S7A13 | ENSG00000164893 | Solute carrier family 7 member 13 (Sodium- |
| | | | | | independent aspartate/glutamate transporter 1) |
| | | | | | (X-amino acid transporter 2) |
| 540 | HLA-A*02:01_LLAQQPIYV | 448 | NALP7 | ENSG00000167634 | NACHT, LRR and PYD domains-containing |
| | | | | | protein 7 (Nucleotide-binding oligomerization |
| | | | | | domain protein 12)(PYRIN-containing APAF1- |
| | | | | | like protein 3) |
| 541 | HLA-B*51:01_LPFTIISM | 341 | TDT | ENSG00000107447 | DNA nucleotidylexotransferase (EC 2.7.7.31) |
| | | | | | (Terminal addition enzyme)(Terminal |
| | | | | | deoxynucleotidyltransfemse)(Terminal |
| | | | | | transferase) |
| 542 | HLA-A*11:01_AVIEHMQEK | 449 | AXDN1 | ENSG00000162779 | Axonemal dynein light chain domain-containing |
| | | | | | protein 1 |
| 543 | HLA-B44:03_EEIIKNIQKLY | 450 | AXDN1 | ENSG00000162779 | Axonemal dynein light chain domain-containing |
| | | | | | protein 1 |
| 544 | HLA-A*02:01_YLIDHPVSL | 451 | AXDN1 | ENSG00000162779 | Axonemal dynein light chain domain-containing |
| | | | | | protein 1 |
| 545 | HLA-A*02:07_YLIDHPVSL | 451 | AXDN1 | ENSG00000162779 | Axonemal dynein light chain domain-containing |
| | | | | | protein 1 |
| 546 | HLA-A*24:02_IYISNSIYF | 452 | CT55 | ENSG00000169551 | Cancer/testis antigen 55 (Tumor antigen BJ- |
| | | | | | HCC-20) |
| 547 | HLA-A*11:01_VVTGNVPLK | 453 | CT55 | ENSG00000169551 | Cancer/testis antigen 55 (Tumor antigen BJ- |
| | | | | | HCC-20) |
| 548 | HLA-A*11:01_ASMSLPPPK | 454 | U3KQD4 | ENSG00000105549 | Testicular haploid-expressed gene protein (Theg |
| | | | | | homolog (Mouse), isoform CRA a) |
| 549 | HLA-C 16:01_VASPRIISL | 455 | U3KQD4 | ENSG00000105549 | Testicular haploid-expressed gene protein (Theg |
| | | | | | homolog (Mouse), isoform CRA a) |
| 550 | HLA-A*29:02_MILDNHALY | 456 | TDT | ENSG00000107447 | DNA nucleotidylexotransferase (EC 2.7.7.31) |
| | | | | | (Terminal addition enzyme)(Terminal |
| | | | | | deoxynucleotidyltransfemse)(Terminal |
| | | | | | transferase) |
| 551 | HLA-A*02:07_TLDEKVDEL | 457 | MAGC1 | ENSG00000155495 | Melanoma-associated antigen C1 (Cancer/testis |
| | | | | | antigen 7.1)(CT7.1)(MAGE-C1 antigen) |
| 552 | HLA-B*35:01_LAFGGHIAF | 458 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 553 | HLA-A*30:02_ALASVGHLY | 288 | ONEC3 | ENSG00000205922 | One cut domain family member 3 (One cut |
| | | | | | homeobox 3)(Transcription factor ONECUT-3) |
| | | | | | (OC-3) |
| 554 | HLA-B*35:01_APLGAAAAY | 459 | NKX24 | ENSG00000125816 | Homeobox protein Nkx-2.4 (Homeobox protein |
| | | | | | NK-2 homolog D) |
| 555 | HLA-A*29:02_GLFTHTIFY | 460 | NDST4 | ENSG00000138653 | Bifunctional heparan sulfate N-deacetylase/N- |
| | | | | | sulfotransferase 4 (EC 2.8.2.8)(Glucosaminyl N- |
| | | | | | deacetylase/N-sulfotransferase 4)(NDST-4)(N- |
| | | | | | heparan sulfate sulfotransferase 4)(N-HSST 4) |
| | | | | | [Includes: Heparan sulfate N-deacetylase 4 (EC |
| | | | | | 3.-.-.-); Heparan sulfate N-sulfotransferase 4 (EC |
| | | | | | 2.8.2.-)] |
| 556 | HLA-B*51:01_VPKAKIITI | 461 | NDST4 | ENSG00000138653 | Bifunctional heparan sulfate N-deacetylase/N- |
| | | | | | sulfotransferase 4 (EC 2.8.2.8)(Glucosaminyl N- |
| | | | | | deacetylase(N-sulfotransferase 4)(NDST-4)(N- |
| | | | | | heparan sulfate sulfotransferase 4)(N-HSST 4) |
| | | | | | [Includes: Heparan sulfate N-deacetylase 4 (EC |
| | | | | | 3.-.-.-); Heparan sulfate N-sulfotransferase 4 (EC |
| | | | | | 2.8.2.-)] |
| 557 | HLA-A*01:01_HTEDKPYKY | 462 | ZN729 | ENSG00000196350 | Zinc finger protein 729 |
| 558 | HLA-A*01:01_HSDSERQYY | 463 | FGF16 | ENSG00000196468 | Fibroblast growth factor 16 (FGF-16) |
| 559 | HLA-A*02:07_SLDWDLHGF | 464 | FGF16 | ENSG00000196468 | Fibroblast growth factor 16 (FGF-16) |
| 560 | HLA-A*31:01_QVKIWFQNR | 110 | HXB1 | ENSG00000120094 | Homeobox protein Hox-B1 (Homeobox protein |
| | | | | | Hox-2I) |
| 561 | HLA-A*11:01_TSLDVPLIGK | 465 | ACHB4 | ENSG00000117971 | Neuronal acetylcholine receptor subunit beta-4 |
| 562 | HLA-B*44:03_EEDLHFVLY | 466 | PRD14 | ENSG00000147596 | PR domain zinc finger protein 14 (EC 2.1.1.-) |
| | | | | | (PR domain-containing protein 14) |
| 563 | HLA-B*46:01_SVVAHLSTY | 61 | ACTL8 | ENSG00000117148 | Actin-like protein 8 (Cancer/testis antigen 57) |
| | | | | | (CT57) |
| 564 | HLA-B*44:03_SEAQDKSKLW | 467 | BRDT | ENSG00000137948 | Brodomain testis-specific protein |
| | | | | | (Cancer/testis antigen 9)(CT9)(RING3-like |
| | | | | | protein) |
| 565 | HLA-A*29:02_AVYNGQWKY | 468 | ESPB1 | ENSG00000169393 | Epididymal sperm-binding protein 1 (Epididymal |
| | | | | | secretory protein 12)(hE12) |
| 566 | HLA-A*11:01_SVTSVFDEK | 469 | ESPB1 | ENSG00000169393 | Epididymal sperm-binding protein 1 (Epididymal |
| | | | | | secretory protein 12)(hE12) |
| 567 | HLA-A*02:01_ALMEVTVYL | 470 | NAL11 | ENSG00000179873 | NACHT, LRR and PYD domains-containing |
| | | | | | protein 11 (Nucleotide-binding oligomerization |
| | | | | | domain protein 17)(PAAD-and NACHT domain- |
| | | | | | containing protein 10)(PYRIN-containing |
| | | | | | APAF1-like protein 6) |
| 568 | HLA-B*44:03_EELANVLPISY | 471 | NAL11 | ENSG00000179873 | NACHT, LRR and PYD domains-containing |
| | | | | | protein 11 (Nucleotide-binding oligomerization |
| | | | | | domain protein 17)(PAAD-and NACHT domain- |
| | | | | | containing protein 10)(PYRIN-containing |
| | | | | | APAF1-like protein 6) |
| 569 | HLA-B*44:03_SEAGLTANQY | 472 | NAL11 | ENSG00000179873 | NACHT, LRR and PYD domains-containing |
| | | | | | protein 11 (Nucleotide-binding oligomerization |
| | | | | | domain protein 17)(PAAD-and NACHT domain- |
| | | | | | containing protein 10)(PYRIN-containing |
| | | | | | APAF1-like protein 6) |
| 570 | HLA-C*02:02_SEAGLTANQY | 472 | NAL11 | ENSG00000179873 | NACHT, LRR and PYD domains-containing |
| | | | | | protein 11 (Nucleotide-binding oligomerization |
| | | | | | domain protein 17)(PAAD-and NACHT domain- |
| | | | | | containing protein 10)(PYRIN-containing |
| | | | | | APAF1-like protein 6) |
| 571 | HLA-A*29:02_VFYILQLAY | 473 | NAL11 | ENSG00000179873 | NACHT, LRR and PYD domains-containing |
| | | | | | protein 11 (Nucleotide-binding oligomerization |
| | | | | | domain protein 17)(PAAD-and NACHT domain- |
| | | | | | containing protein 10)(PYRIN-containing |
| | | | | | APAF1-like protein 6) |
| 572 | HLA-A*11:01_STVDPMKLYEK | 474 | GCM1 | ENSG00000137270 | Chorion-specific transcription factor GCMa |
| | | | | | (hGCMa)(GCM motif protein 1)(Glia1 cells |
| | | | | | missing homolog 1) |
| 573 | HLA-B35:01_SAVTPGPYY | 366 | SPT22 | ENSG00000141255 | Spermatogenesis-associated protein 22 (Testis |
| | | | | | development protein NYD-SP20) |
| 574 | HLA-A02:07_YLDLRNTGL | 475 | LRC52 | ENSG00000162763 | Leucine-rich repeat-containing protein 52 (BK |
| | | | | | channel auxilialy gamma subunit LRRC52) |
| 575 | HLA-A*11:01_ATIDVTTVER | 476 | PCDC1 | ENSG00000248383 | Protocadherin alpha-C 1 (PCDH-alpha-C1) |
| 576 | HLA-B*51:01_DPLELHKI | 477 | PCDC1 | ENSG00000248383 | Protocadherin alpha-C 1 (PCDH-alpha-C1) |
| 577 | HLA-A*02:07_TVADPLPQV | 230 | A0A1B0GTI6 | ENSG00000268655 | HCG1796489 |
| 578 | HLA-B*35:01_SPEAGLAEY | 478 | FHL17 | ENSG00000132446 | Ferritin heavy polypeptide-like 17 (Cancer/testis |
| | | | | | antigen 38)(CT38) |
| 579 | HLA-B*51:01_IPMDGTAVI | 479 | CALI | ENSG00000185972 | Calicin |
| 580 | HLA-A*01:01_GSEVSFLEY | 480 | PIWL1 | ENSG00000125207 | Piwi-like protein 1 (EC 3.1.26.-) |
| 581 | HLA-B*44:02_KEFGGPFFW | 409 | CTSRD | ENSG00000174898 | Cation channel sperm-associated protein subunit |
| | | | | | delta (CatSper-delta)(CatSperdelta) |
| | | | | | (Transmembrane protein 146) |
| 582 | HLA-A*11:01_AAGALPLLK | 481 | GSX2 | ENSG00000180613 | GS homeobox 2 (Genetic-screened homeobox 2) |
| | | | | | (Homeobox protein GSH-2) |
| 583 | HLA-A*11:01_ATYLNLSEK | 482 | GSX2 | ENSG00000180613 | GS homeobox 2 (Genetic-screened homeobox 2) |
| | | | | | (Homeobox protein GSH-2) |
| 584 | HLA-B*51:01_MPPPLVMSV | 483 | GSX2 | ENSG00000180613 | GS homeobox 2 (Genetic-screened homeobox 2) |
| | | | | | (Homeobox protein GSH-2) |
| 585 | HLA-A*31:01_QVKIWFQNR | 110 | GSX2 | ENSG00000180613 | GS homeobox 2 (Genetic-screened homeobox 2) |
| | | | | | (Homeobox protein GSH-2) |
| 586 | HLA-B*51:01_VPPWNPQLI | 484 | ADIG | ENSG00000182035 | Adipogenin |
| 587 | HLA-C*04:01_MFDNGSFL | 485 | FOXE3 | ENSG00000186790 | Forkhead box protein E3 (Forkhead-related |
| | | | | | protein FKHL12)(Forkhead-related transcription |
| | | | | | factor 8)(FREAC-8) |
| 588 | HLA-A*11:01_KSGDLVFAK | 486 | HDGL1 | ENSG00000112273 | Hepatoma-derived growth factor-like protein 1 |
| | | | | | (PWWP domain-containing protein 1) |
| 589 | HLA-A*31:01_ATKSGLVVR | 487 | GCNT7 | ENSG00000124091 | Beta-1,3-galactosyl-O-glycosyl-glycoprotein |
| | | | | | beta-1,6-N-acetylgluco saminyltransferase 7 (EC |
| | | | | | 2.4.1.-) |
| 590 | HLA-B*51:01_LAPPIGNSI | 488 | F71F1 | ENSG00000135248 | Protein FAM71F1 (Protein FAM137A)(Testis |
| | | | | | development protein NYD-SP18) |
| 591 | HLA-B*44:03_EEGEDRDGHAW | 489 | NALP7 | ENSG00000167634 | NACHT, LRR and PYD domains-containing |
| | | | | | protein 7 (Nucleotide-binding oligomerization |
| | | | | | domain protein 12)(PYRIN-containing APAF1- |
| | | | | | like protein 3) |
| 592 | HLA-A*24:02_EYLKDPVTI | 490 | TRI60 | ENSG00000176979 | Tripartite motif-containing protein 60 (RING |
| | | | | | finger protein 129)(RING finger protein 33) |
| 593 | HLA-B*44:03_SEFEQIRLF | 491 | TRI60 | ENSG00000176979 | Tripartite motif-containing protein 60 (RING |
| | | | | | finger protein 129)(RING finger protein 33) |
| 594 | HLA-B*18:01SEVSFLEYY | 94 | PIWL1 | ENSG00000125207 | Piwi-like protein 1 (BC 3.1.26.-) |
| 595 | HLA-A*11:01_AGISSTITR | 492 | SAGE1 | ENSG00000181433 | Sarcoma antigen 1 (Cancer/testis antigen 14) |
| | | | | | (CT14) |
| 596 | HLA-B*18:01_EETRVLAF | 493 | FOXR2 | ENSG00000189299 | Forkhead box protein R2 (Forkhead box protein |
| | | | | | N6) |
| 597 | HLA-A*11:01_SSEQSPLQK | 494 | FOXR2 | ENSG00000189299 | Forkhead box protein R2 (Forkhead box protein |
| | | | | | N6) |
| 598 | HLA-A*02:01_ALYSGDLHAA | 495 | E5RK72 | ENSG00000215262 | Potassium channel subfamily U member 1 |
| 599 | HLA-B*18:01_EEFSLQKSY | 496 | E5RK72 | ENSG00000215262 | Potassium channel subfamily U member 1 |
| 600 | HLA-B*44:03_EEFSLQKSY | 496 | E5RK72 | ENSG00000215262 | Potassium channel subfamily U member 1 |
| 601 | HLA-C*02:02_EEFSLQKSY | 496 | E5RK72 | ENSG00000215262 | Potassium channel subfamily U member 1 |
| 602 | HLA-A*02:07_FLDSLLATL | 497 | E5RK72 | ENSG00000215262 | Potassium channel subfamily U member 1 |
| 603 | HLA-B*35:01_HAEDISNIM | 498 | E5RK72 | ENSG00000215262 | Potassium channel subfamily U member 1 |
| 604 | HLA-A*11:01_STVGFGDVVAK | 499 | E5RK72 | ENSG00000215262 | Potassium channel subfamily U member 1 |
| 605 | HLA-B*35:01_TAFSTGTVF | 500 | E5RK72 | ENSG00000215262 | Potassium channel subfamily U member 1 |
| 606 | HLA-A*02:07_TVDSVTAFL | 501 | E5RK72 | ENSG00000215262 | Potassium channel subfamily U member 1 |
| 607 | HLA-A*11:01_GVAALTPVQK | 502 | ANHX | ENSG00000227059 | Anomalous homeobox protein |
| 608 | HLA-A*02:01_HLLDNADVAL | 503 | ANHX | ENSG00000227059 | Anomalous homeobox protein |
| 609 | HLA-A*02:07_LLDNADVAL | 504 | ANHX | ENSG00000227059 | Anomalous homeobox protein |
| 610 | HLA-C*02:02_IEAELHISY | 254 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provims ancestral Env |
| | | | | | polyprotein)[Cleaved into: Surface protein (SU); |
| | | | | | Transmembrane protein (TM)] |
| 611 | HLA-B*18:01_TETPGTAY | 505 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 612 | HLA-C*01:02_TGPSGNAL | 506 | S22AD | ENSG00000172940 | Solute carrier family 22 member 13 (Organic |
| | | | | | cation transporter-like 3)(ORCTL-3) |
| 613 | HLA-A*11:01_ASFLTTVFK | 507 | NALP9 | ENSG00000185792 | NACHT, LRR and PYD domains-containing |
| | | | | | protein 9 (Nucleotide-binding oligomerization |
| | | | | | domain protein 6)(PYRIN and NACHT- |
| | | | | | containing protein 12) |
| 614 | HLA-A*24:02_IYIGNIEHL | 508 | NALP9 | ENSG00000185792 | NACHT, LRR and PYD domains-containing |
| | | | | | protein 9 (Nucleotide-binding oligomerization |
| | | | | | domain protein 6)(PYRIN and NACHT- |
| | | | | | containing protein 12) |
| 615 | HLA-B*51:01_DAFPISLI | 509 | CXA10 | ENSG00000135355 | Gap junction alpha-10 protein (Connexin-62) |
| | | | | | (Cx62) |
| 616 | HLA-A*30:02_KTLAEISDRY | 510 | X1WI33 | ENSG00000170788 | DPY30 domain-containing protein 1 (Fragment) |
| 617 | HLA-A*11:01_VTMEQLRQK | 511 | X1WI33 | ENSG00000170788 | DPY30 domain-containing protein 1 (Fragment) |
| 618 | HLA-B*08:01_HAYHKVTL | 512 | CF010 | ENSG00000204296 | Uncharacterized protein C6orf10 |
| 619 | HLA-A*01:01_QSEMYISRY | 513 | CF010 | ENSG00000204296 | Uncharacterized protein C6orf10 |
| 620 | HLA-A*01:01_SSEQSARLLDY | 514 | CF010 | ENSG00000204296 | Uncharacterized protein C6orf10 |
| 621 | HLA-A*11:01_ATAQPSQVR | 515 | FHL17 | ENSG00000132446 | Ferritin heavy polypeptide-like 17 (Cancer/testis |
| | | | | | antigen 38)(CT38) |
| 622 | HLA-A*26:01_DIISEQKVSEF | 516 | RNF17 | ENSG00000132972 | RING finger protein 17 (Tudor domain- |
| | | | | | containing protein 4) |
| 623 | HLA-A*02:01_ALLGILISV | 517 | IZUM2 | ENSG00000161652 | Izumo sperm-egg fusion protein 2 |
| 624 | HLA-A*24:02_LYTKAHETF | 518 | MSLNL | ENSG00000162006 | Mesothelin-like protein (Pre-pro-megakroyocyte- |
| | | | | | potentiating-factor-like) |
| 625 | HLA-A*24:02_SYLLGWTTF | 519 | ESPB1 | ENSG00000169393 | Epididymal sperm-binding protein 1 (Epididymal |
| | | | | | secretory protein 12)(hE12) |
| 626 | HLA-C*02:02_AEIEPVSAV | 520 | ZFP42 | ENSG00000179059 | Zinc finger protein 42 homolog (Zfp-42) |
| | | | | | (Reduced expression protein 1)(REX-1)(hREX- |
| | | | | | 1)(Zinc finger protein 754) |
| 627 | HLA-A*11:01_VIIDHGSGFLK | 521 | ACTL8 | ENSG00000117148 | Actin-like protein 8 (Cancer/testis antigen 57) |
| | | | | | (CT57) |
| 628 | HLA-B*51:01_MPYTEAVI | 522 | CP2AD | ENSG00000197838 | Cytochrome P450 2A13 (EC 1.14.14.1) |
| | | | | | (CYPIIA13) |
| 629 | HLA-B*35:01_LPVPLDSAF | 523 | ASCL4 | ENSG00000187855 | Achaete-scute homolog 4 (ASH-4)(hASH4) |
| | | | | | (Achaete-scute-like protein 4)(Class A basic |
| | | | | | helix-loop-helix protein 44)(bHLHa44) |
| 630 | HLA-B*44:03_QELLERQAW | 524 | ASCL4 | ENSG00000187855 | Achaete-scute homolog 4 (ASH-4)(hASH4) |
| | | | | | (Achaete-scute-like protein 4)(Class A basic |
| | | | | | helix-loop-helix protein 44)(bHLHa44) |
| 631 | HLA-B*51:01_DAYLSYTKV | 525 | IRPL2 | ENSG00000189108 | X-linked interleukin-1 receptor accessory protein- |
| | | | | | like 2 (IL-1 receptor accessory protein-like 2) |
| | | | | | (IL-1-RAPL-2)(IL-IRAPL-2)(IL 1RAPL-2) |
| | | | | | (IL1RAPL-2-related protein)(Interleukin-1 |
| | | | | | receptor 9)(IL-1R-9)(11,-1R9)(Three |
| | | | | | immunoglobulin domain-containing IL-1 |
| | | | | | receptor-related 1)(TIGIRR-1) |
| 632 | HLA-A*11:01_STNLKMVSK | 526 | IRPL2 | ENSG00000189108 | X-linked interleukin-1 receptor accessory protein- |
| | | | | | like 2 (IL-1 receptor accessory protein-like 2) |
| | | | | | (IL-1-RAPL-2)(IL-IRAPL-2)(IL 1RAPL-2) |
| | | | | | (IL1RAPL-2-related protein)(Interleukin-1 |
| | | | | | receptor 9)(IL-1R-9)(11,-1R9)(Three |
| | | | | | immunoglobulin domain-containing IL-1 |
| | | | | | receptor-related 1)(TIGIRR-1) |
| 633 | HLA-A*02:07_ALDPPVDVF | 527 | LN28A | ENSG00000131914 | Protein lin-28 homolog A (Lin-28A)(Zinc finger |
| | | | | | CCHC domain-containing protein 1) |
| 634 | HLA-B*35:01_YPLSPTISL | 528 | RBM46 | ENSG00000151962 | Probable RNA-binding protein 46 (Cancer/testis |
| | | | | | antigen 68)(CT68)(RNA-binding motif protein |
| | | | | | 46) |
| 635 | HLA-A*03:01_HILTHANTNK | 529 | ZFP42 | ENSG00000179059 | Zinc finger protein 42 homolog (Zfp-42) |
| | | | | | (Reduced expression protein 1)(REX-1)(hREX- |
| | | | | | 1)(Zinc finger protein 754) |
| 636 | HLA-A*26:01_SVTTYTGSY | 530 | CD051 | ENSG00000237136 | Uncharacterized protein C4orf51 |
| 637 | HLA-A*29:02_AFLIIVFSY | 531 | RXFP2 | ENSG00000133105 | Relaxin receptor 2 (G-protein coupled receptor |
| | | | | | 106)(G-protein coupled receptor affecting |
| | | | | | testicular descent)(Leucine-rich repeat- |
| | | | | | containing G-protein coupled receptor 8)(Relaxin |
| | | | | | family peptide receptor 2) |
| 638 | HLA-A*29:02_FFVGIFDIKY | 532 | RXFP2 | ENSG00000133105 | Relaxin receptor 2 (G-protein coupled receptor |
| | | | | | 106)(G-protein coupled receptor affecting |
| | | | | | testicular descent)(Leucine-rich repeat- |
| | | | | | containing (G-protein coupled receptor 8)(Relaxin |
| | | | | | family peptide receptor 2) |
| 639 | HLA-A*29:02_FVGIFDIKY | 533 | RXFP2 | ENSG00000133105 | Relaxin receptor 2 (G-protein coupled receptor |
| | | | | | 106)(G-protein coupled receptor affecting |
| | | | | | testicular descent)(Leucine-rich repeat- |
| | | | | | containing (G-protein coupled receptor 8)(Relaxin |
| | | | | | family peptide receptor 2) |
| 640 | HLA-A*02:07_ILDDNPITRI | 534 | RXFP2 | ENSG00000133105 | Relaxin receptor 2 (G-protein coupled receptor |
| | | | | | 106)(G-protein coupled receptor affecting |
| | | | | | testicular descent)(Leucine-rich repeat- |
| | | | | | containing G-protein coupled receptor 8)(Relaxin |
| | | | | | family peptide receptor 2) |
| 641 | HLA-A*24:02_LYTLTTNFF | 535 | RXFP2 | ENSG00000133105 | Relaxin receptor 2 (G-protein coupled receptor |
| | | | | | 106)(G-protein coupled receptor affecting |
| | | | | | testicular descent)(Leucine-rich repeat- |
| | | | | | containing G-protein coupled receptor 8)(Relaxin |
| | | | | | family peptide receptor 2) |
| 642 | HLA-B*35:01_MPLTDGISSF | 536 | RXFP2 | ENSG00000133105 | Relaxin receptor 2 (G-protein coupled receptor |
| | | | | | 106)(G-protein coupled receptor affecting |
| | | | | | testicular descent)(Leucine-rich repeat- |
| | | | | | containing G-protein coupled receptor 8)(Relaxin |
| | | | | | family peptide receptor 2) |
| 643 | HLA-B*44:03_TEDIGSKGY | 537 | RXFP2 | ENSG00000133105 | Relaxin receptor 2 (G-protein coupled receptor |
| | | | | | 106)(G-protein coupled receptor affecting |
| | | | | | testicular descent)(Leucine-rich repeat- |
| | | | | | containing G-protein coupled receptor 8)(Relaxin |
| | | | | | family peptide receptor 2) |
| 644 | HLA-A*29:02_GYWGVRLKY | 538 | KCNV2 | ENSG00000168263 | Potassium voltage-gated channel subfamily V |
| | | | | | member 2 (Voltage-gated potassium channel |
| | | | | | subunit Kv8.2) |
| 645 | HLA-B*18:01_LEEKMIAAY | 539 | RNF17 | ENSG00000132972 | RING finger protein 17 (Tudor domain- |
| | | | | | containing protein 4) |
| 646 | HLA-A*02:07_SLDEALQRV | 540 | RNF17 | ENSG00000132972 | RING finger protein 17 (Tudor domain- |
| | | | | | containing protein 4) |
| 647 | HLA-A*29:02_AVWPLELAY | 541 | UBP41 | ENSG00000161133 | Putative ubiquitin carboxyl-terminal hydrolase 41 |
| | | | | | (EC 3.4.19.12)(Deubiquitinating enzyme 41) |
| | | | | | (Ubiquitin thioesterase 41)(Ubiquitin-specific- |
| | | | | | processing protease 41) |
| 648 | HLA-B*46:01_LAFGGHIAF | 458 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 649 | HLA-A*01:01_DTELGSSEY | 542 | PERL | ENSG00000167419 | Lactoperoxidase (LPO)(EC 1.11.1.7)(Salivary |
| | | | | | peroxidase)(SPO) |
| 650 | HLA-A*02:07_GLPDIMKPIKL | 543 | GP139 | ENSG00000180269 | Probable G-protein coupled receptor 139 (G(q)- |
| | | | | | coupled orphan receptor GPRg1)(G-protein- |
| | | | | | coupled receptor PGR3) |
| 651 | HLA-B*51:01_LPANILTVI | 544 | GP139 | ENSG00000180269 | Probable G-protein coupled receptor 139 (G(q)- |
| | | | | | coupled orphan receptor GPRg1)(G-protein- |
| | | | | | coupled receptor PGR3) |
| 652 | HLA-A*02:01_SLAETPASA | 545 | GALP | ENSG00000197487 | Galanin-like peptide |
| 653 | HLA-A*30:02_SIQQLVPEY | 546 | C144L | ENSG00000205212 | Putative coiled-coil domain-containing protein |
| | | | | | 144 N-terminal-like |
| 654 | HLA-B*18:01_DELEKQIVY | 547 | E9PBZ7 | ENSG00000242715 | Coiled-coil domain-containing protein 169 |
| 655 | HLA-B*51:01_FPLPLAREV | 548 | PERL | ENSG00000167419 | Lactoperoxidase (LPO)(EC 1.11.1.7)(Salivary |
| | | | | | peroxidase)(SPO) |
| 656 | HLA-A*11:01_SSMALPSPHK | 549 | FOXR1 | ENSG00000176302 | Forkhead box protein R1 (Forkhead box protein |
| | | | | | N5) |
| 657 | HLA-A*26:01_DVAEAIAFF | 550 | A0A1B0GVHE | ENSG00000226792 | Long intergenic non-protein coding RNA 371 |
| 658 | HLA-A*26:01_DTAVLITRY | 551 | ATS20 | ENSG00000173157 | A disintegrin and metalloproteinase with |
| | | | | | thrombospondin motifs 20 (ADAM-TS 20) |
| | | | | | (ADAM-TS20)(ADAMTS-20)(EC 3.4.24.-) |
| 659 | HLA-C*04:01_NFDGATTL | 552 | ATS20 | ENSG00000173157 | A disintegrin and metalloproteinase with |
| | | | | | thrombospondin motifs 20 (ADAM-TS 20) |
| | | | | | (ADAM-TS20)(ADAMTS-20)(EC 3.4.24.-) |
| 660 | HLA-A*03:01_STNLPLTQK | 553 | ATS20 | ENSG00000173157 | A disintegrin and metalloproteinase with |
| | | | | | thrombospondin motifs 20 (ADAM-TS 20) |
| | | | | | (ADAM-TS20)(ADAMTS-20)(EC 3.4.24.-) |
| 661 | HLA-A*11:01_STNLPLTQK | 553 | ATS20 | ENSG00000173157 | A disintegrin and metalloproteinase with |
| | | | | | thrombospondin motifs 20 (ADAM-TS 20) |
| | | | | | (ADAM-TS20)(ADAMTS-20)(EC 3.4.24.-) |
| 662 | HLA-A*24:02_NYFIDPVTI | 554 | TRI48 | ENSG00000150244 | Tripartite motif-containing protein 48 (RING |
| | | | | | finger protein 101) |
| 663 | HLA-B*51:01_DPITFSFI | 555 | UROL1 | ENSG00000177398 | Uromodulin-like 1 (Olfactorin) |
| 664 | HLA-A*26:01_EVISVQVQDV | 556 | UROL1 | ENSG00000177398 | Uromodulin-like 1 (Olfactorin) |
| 665 | HLA-A*02:01_SLFESLEYL | 557 | ZFP42 | ENSG00000179059 | Zinc finger protein 42 homolog (Zfp-42) |
| | | | | | (Reduced expression protein 1)(REX-1)(hREX- |
| | | | | | 1)(Zinc finger protein 754) |
| 666 | HLA-B*46:01_FLITQATAY | 321 | NBPF6 | ENSG00000186086 | Neuroblastoma breakpoint family member 6 |
| 667 | HLA-A*03:01_RTFPITGLRY | 444 | DYTN | ENSG00000232125 | Dystrotelin |
| 668 | HLA-C*02:02_AELQASLSKY | 558 | TDRD1 | ENSG00000095627 | Tudor domain-containing protein 1 (Cancer/testis |
| | | | | | antigen 41.1)(CT41.1) |
| 669 | HLA-A*29:02_IFSDQETFY | 559 | PRD14 | ENSG00000147596 | PR domain zinc finger protein 14 (EC 2.1.1.-) |
| | | | | | (PR domain-containing protein 14) |
| 670 | HLA-A*03:01_AVYNSPQFKK | 560 | FBX39 | ENSG00000177294 | F-box only protein 39 |
| 671 | HLA-A*11:01_AVYNSPQFKK | 560 | FBX39 | ENSG00000177294 | F-box only protein 39 |
| 672 | HLA-A*11:01_AVYNSPQFK | 561 | FBX39 | ENSG00000177294 | F-box only protein 39 |
| 673 | HLA-B*51:01_SAYGNATSV | 562 | ZPLD1 | ENSG00000170044 | Zona pellucida-like domain-containing protein 1 |
| | | | | | (ZP domain-containing protein 1) |
| 674 | HLA-A*26:01_FTVDSNQQTY | 563 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 675 | HLA-A*30:02_AVAPHHSGVY | 564 | NDST4 | ENSG00000138653 | Bifunctional heparan sulfate N-deacetylase/N- |
| | | | | | sulfotransferase 4 (EC 2.8.2.8)(Glucosaminyl N- |
| | | | | | deacetylase/N-sulfotransferase 4)(NDST-4)(N- |
| | | | | | heparan sulfate sulfotransferase 4)(N-HSST 4) |
| | | | | | [Includes: Heparan sulfate N-deacetylase 4 (EC |
| | | | | | 3.-.-.-); Heparan sulfate N-sulfotransferase 4 (EC |
| | | | | | 2.8.2.-)] |
| 676 | HLA-A*11:01_SVSIIGHIK | 565 | NDST4 | ENSG00000138653 | Bifunctional heparan sulfate N-deacetylase/N- |
| | | | | | sulfotransferase 4 (EC 2.8.2.8)(Glucosaminyl N- |
| | | | | | deacetylase/N-sulfotransferase 4)(NDST-4)(N- |
| | | | | | heparan sulfate sulfotransferase 4)(N-HSST 4) |
| | | | | | [Includes: Heparan sulfate N-deacetylase 4 (EC |
| | | | | | 3.-.-.-); Heparan sulfate N-sulfotransferase 4 (EC |
| | | | | | 2.8.2.-)] |
| 677 | HLA-A*31:01_QSFPPKVNR | 566 | NALP9 | ENSG00000185792 | NACHT, LRR and PYD domains-containing |
| | | | | | protein 9 (Nucleotide-binding oligomerization |
| | | | | | domain protein 6)(PYRIN and NACHT- |
| | | | | | containing protein 12) |
| 678 | HLA-A*02:01_FIHEDLNTV | 567 | RNS10 | ENSG00000182545 | Inactive ribonuclease-like protein 10 |
| 679 | HLA-A*29:02_NFFEEVFIY | 568 | NALP9 | ENSG00000185792 | NACHT, LRR and PYD domains-containing |
| | | | | | protein 9 (Nucleotide-binding oligomerization |
| | | | | | domain protein 6)(PYRIN and NACHT- |
| | | | | | containing protein 12) |
| 680 | HLA-B*44:02_EEMQIQKSQW | 569 | NTM2G | ENSG00000188152 | NUT family member 2G |
| 681 | HLA-B*44:03_EEMQIQKSQW | 569 | NTM2G | ENSG00000188152 | NUT family member 2G |
| 682 | HLA-A*01:01_TSDPGLLSY | 570 | NTM2G | ENSG00000188152 | NUT family member 2G |
| 683 | HLA-B*18:01_EESVLVGY | 571 | TDRD1 | ENSG00000095627 | Tudor domain-containing protein 1 (Cancer/testis |
| | | | | | antigen 41.1)(CT41.1) |
| 684 | HLA-B*51:01_DAHNTHVGI | 572 | WNT9B | ENSG00000158955 | Protein Wnt-9b (Protein Wnt-14b)(Protein Wnt- |
| | | | | | 15) |
| 685 | HLA-A*29:02_GFKETAFLY | 573 | WNT9B | ENSG00000158955 | Protein Wnt-9b (Protein Wnt-14b)(Protein Wnt- |
| | | | | | 15) |
| 686 | HLA-A*24:02_VYMEDSPSF | 574 | WNT9B | ENSG00000158955 | Protein Wnt-9b (Protein Wnt-14b)(Protein Wnt- |
| | | | | | 15) |
| 687 | HLA-A*11:01_AVAAKMEVK | 575 | SG1D1 | ENSG00000168515 | Secretoglobin family 1D member 1 (Lipophilin- |
| | | | | | A) |
| 688 | HLA-A*29:02_AFLKMIYSY | 576 | GPC6A | ENSG00000173612 | G-protein coupled receptor family C group 6 |
| | | | | | member A (hGPRC6A)(G-protein coupled |
| | | | | | receptor GPCR33)(hGPCR33) |
| 689 | HLA-B*18:01_NEAKFITF | 577 | GPC6A | ENSG00000173612 | G-protein coupled receptor family C group 6 |
| | | | | | member A (hGPRC6A)(G-protein coupled |
| | | | | | receptor GPCR33)(hGPCR33) |
| 690 | HLA-B*51:01_LPKLPKPYI | 578 | PSG1 | ENSG00000231924 | Pregnancy-specific beta-1-glycoprotein 1 (PS- |
| | | | | | beta-G-1)(PSBG-1)(Pregnancy-specific |
| | | | | | glycoprotein 1)(CD66 antigen-like family |
| | | | | | member F)(Fetal liver non-specific cross-reactive |
| | | | | | antigen 1/2)(FL-NCA-1/2)(PSG95)(Pregnancy- |
| | | | | | specific beta-1 glycoprotein C/D)(PS-beta-C/D) |
| | | | | | (CD antigen CD661) |
| 691 | HLA-B*51:01_LPTTAQVTI | 579 | PSG1 | ENSG00000231924 | Pregnancy-specific beta-1-glycoprotein 1 (PS- |
| | | | | | beta-G-1)(PSBG-1)(Pregnancy-specific |
| | | | | | glycoprotein 1)(CD66 antigen-like family |
| | | | | | member F)(Fetal liver non-specific cross-reactive |
| | | | | | antigen 1/2)(FL-NCA-1/2)(PSG95)(Pregnancy- |
| | | | | | specific beta-1 glycoprotein C/D)(PS-beta-C/D) |
| | | | | | (CD antigen CD66f) |
| 692 | HLA-B*51:01_LPYYSTSII | 580 | ADAM7 | ENSG00000069206 | Disintegrin and metalloproteinase domain- |
| | | | | | containing protein 7 (ADAM 7)(Sperm |
| | | | | | maturation-related glycoprotein GP-83) |
| 693 | HLA-B*51:01_LPYYSTSI | 581 | ADAM7 | ENSG00000069206 | Disintegrin and metalloproteinase domain- |
| | | | | | containing protein 7 (ADAM 7)(Sperm |
| | | | | | maturation-related glycoprotein GP-83) |
| 694 | HLA-A*11:01_VTLAKPVNK | 582 | RBM46 | ENSG00000151962 | Probable RNA-binding protein 46 (Cancer/testis |
| | | | | | antigen 68)(CT68)(RNA-binding motif protein |
| | | | | | 46) |
| 695 | HLA-A*11:01_STEPGISNIK | 583 | CT55 | ENSG00000169551 | Cancer/testis antigen 55 (Tumor antigen BJ- |
| | | | | | HCC-20) |
| 696 | HLA-A*03:01_VVTGNVPLK | 453 | CT55 | ENSG00000169551 | Cancer/testis antigen 55 (Tumor antigen BJ- |
| | | | | | HCC-20) |
| 697 | HLA-A*30:02_DLNDVTHVY | 584 | DDX53 | ENSG00000184735 | Probable ATP-dependent RNA helicase DDX53 |
| | | | | | (EC 3.6.4.13)(Cancer-associated gene protein) |
| | | | | | (Cancer/testis antigen 26)(CT26)(DEAD box |
| | | | | | protein 53)(DEAD box protein CAGE) |
| 698 | HLA-A*31:01_RVQVWFQNR | 367 | ESX1 | ENSG00000123576 | Homeobox protein ESX1 (Extraembryonic, |
| | | | | | spermatogenesis, homeobox 1)[Cleaved into: |
| | | | | | Homeobox protein ESX1-N; Homeobox protein |
| | | | | | ESX1-C] |
| 699 | HLA-A*03:01_KLFIPQITTK | 585 | PSG8 | ENSG00000124467 | Pregnancy-specific beta-1-glycoprotein 8 (PS- |
| | | | | | beta-G-8)(PSBG-8)(Pregnancy-specific |
| | | | | | glycoprotein 8) |
| 700 | HLA-B*51:01_LPKLPKPYI | 578 | PSG8 | ENSG00000124467 | Pregnancy-specific beta-1-glycoprotein 8 (PS- |
| | | | | | beta-G-8)(PSBG-8)(Pregnancy-specific |
| | | | | | glycoprotein 8) |
| 701 | HLA-B*51:01_YPKLPKPYI | 586 | PSG8 | ENSG00000124467 | Pregnancy-specific beta-1-glycoprotein 8 (PS- |
| | | | | | beta-G-8)(PSBG-8)(Pregnancy-specific |
| | | | | | glycoprotein 8) |
| 702 | HLA-B*35:01_LPLVTVVY | 587 | S7A13 | ENSG00000164893 | Solute carrier family 7 member 13 (Sodium- |
| | | | | | independent aspartate/glutamate transporter 1) |
| | | | | | (X-amino acid transporter 2) |
| 703 | HLA-A*24:02_NYGVLHVTF | 588 | NAL11 | ENSG00000179873 | NACHT, LRR and PYD domains-containing |
| | | | | | protein 11 (Nucleotide-binding oligomerization |
| | | | | | domain protein 17)(PAAD-and NACHT domain- |
| | | | | | containing protein 10)(PYRIN-containing |
| | | | | | APAF1-like protein 6) |
| 704 | HLA-B*18:01_DETEIRSFF | 589 | DAZL | ENSG00000092345 | Deleted in azoospermia-like (DAZ homolog) |
| | | | | | (DAZ-like autosomal)(Deleted in azoospermia- |
| | | | | | like 1)(SPGY-like-autosomal) |
| 705 | HLA-B*18:01_DETEIRSF | 590 | DAZL | ENSG00000092345 | Deleted in azoospermia-like (DAZ homolog) |
| | | | | | (DAZ-like autosomal)(Deleted in azoospermia- |
| | | | | | like 1)(SPGY-like-autosomal) |
| 706 | HLA-B*35:01_SPVQVITGY | 591 | DAZL | ENSG00000092345 | Deleted in azoospermia-like (DAZ homolog) |
| | | | | | (DAZ-like autosomal)(Deleted in azoospermia- |
| | | | | | like 1)(SPGY-like-autosomal) |
| 707 | HLA-B*51:01_DAYIPGGPLTV | 592 | OVOL3 | ENSG00000105261 | Putative transcription factor ovo-like protein 3 |
| 708 | HLA-A*30:02_KVHGQPASYAY | 593 | OVOL3 | ENSG00000105261 | Putative transcription factor ovo-like protein 3 |
| 709 | HLA-A*30:02_KVHGQPASY | 594 | OVOL3 | ENSG00000105261 | Putative transcription factor ovo-like protein 3 |
| 710 | HLA-B*35:01_SPAPSLESY | 595 | MSGN1 | ENSG00000151379 | Mesogenin-1 (Paraxial mesoderm-specific |
| | | | | | mesogeninl)(pMesogeninl)(pMsgn1) |
| 711 | HLA-A*02:01_TLADALHTL | 596 | MSGN1 | ENSG00000151379 | Mesogenin-1 (Paraxial mesoderm-specific |
| | | | | | mesogeninl)(pMesogeninl)(pMsgn1) |
| 712 | HLA-B*51:01_LPAVQAPVI | 597 | TEKT5 | ENSG00000153060 | Tektin-5 |
| 713 | HLA-B*08:01_YSAARAVSL | 598 | NPBW1 | ENSG00000183729 | Neuropeptides B/W receptor type 1 (G-protein |
| | | | | | coupled receptor 7) |
| 714 | HLA-A*24:02_VYFGHDSELF | 599 | NALP9 | ENSG00000185792 | NACHT, LRR and PYD domains-containing |
| | | | | | protein 9 (Nucleotide-binding oligomerization |
| | | | | | domain protein 6)(PYRIN and NACHT- |
| | | | | | containing protein 12) |
| 715 | HLA-B*51:01_IGYVTPDI | 600 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 716 | HLA-B*51:01_LPLQFLVV | 601 | WFD13 | ENSG00000168634 | WAP four-disulfide core domain protein 13 |
| 717 | HLA-B*51:01_LPPTSSISI | 602 | AR13A | ENSG00000174225 | ADP-ribosylation factor-like protein 13A |
| 718 | HLA-A*24:02_PYPDVTFTL | 603 | ACHA9 | ENSG00000174343 | Neuronal acetylcholine receptor subunit alpha-9 |
| | | | | | (Nicotinic acetylcholine receptor subunit alpha-9) |
| | | | | | (NACHR alpha-9) |
| 719 | HLA-B*44:03_GEYPDYQQW | 604 | CRGB | ENSG00000182187 | Gamma-crystallin B (Gamma-B-crystallin) |
| | | | | | (Gamma-crystallin 1-2) |
| 720 | HLA-C*02:02_GEYPDYQQW | 604 | CRGB | ENSG00000182187 | Gamma-crystallin B (Gamma-B-crystallin) |
| | | | | | (Gamma-criystallin 1-2) |
| 721 | HLA-B*35:01_MPVPGQHSM | 605 | AMELX | ENSG00000125363 | Amelogenin, X isoform |
| 722 | HLA-C*16:01_AAVQRAAEL | 606 | TF2LX | ENSG00000153779 | Homeobox protein TGIF2LX (TGF-beta-induced |
| | | | | | transcription factor 2-like protein)(TUB- |
| | | | | | induced factor 2-like protein, X-linked)(TGIF- |
| | | | | | like on the X) |
| 723 | HLA-A*29:02_AIFQGYFAY | 607 | S7A13 | ENSG00000164893 | Solute carrier family 7 member 13 (Sodium- |
| | | | | | independent aspartate/glutamate transporter 1) |
| | | | | | (X-amino acid transporter 2) |
| 724 | HLA-A*11:01_AQFSSSAIQK | 608 | SOX14 | ENSG00000168875 | Transcription factor SOX-14 (Protein SOX-28) |
| 725 | HLA-A*29:02_AFAPTFLVY | 609 | S22AO | ENSG00000197658 | Solute carrier family 22 member 24 |
| 726 | HLA-B*51:01_DAVRIKTSI | 610 | S22AO | ENSG00000197658 | Solute carrier family 22 member 24 |
| 727 | HLA-B*51:01_FPILAVPVI | 611 | S22AO | ENSG00000197658 | Solute carrier family 22 member 24 |
| 728 | HLA-C*16:01_SASVHHNEL | 612 | S22AO | ENSG00000197658 | Solute carrier family 22 member 24 |
| 729 | HLA-A*11:01_SVSGLVLSH | 613 | S22AO | ENSG00000197658 | Solute carrier family 22 member 24 |
| 730 | HLA-B*35:01_HPAVTPDAY | 614 | AP2D | ENSG00000008197 | Transcription factor AP-2-delta (AP2-delta) |
| | | | | | (Activating enhancer-binding protein 2-delta) |
| | | | | | (Transcription factor AP-2-beta-like 1) |
| 731 | HLA-B*35:01_SVANSTVAY | 615 | AP2D | ENSG00000008197 | Transcription factor AP-2-delta (AP2-delta) |
| | | | | | (Activating enhancer-binding protein 2-delta) |
| | | | | | (Transcription factor AP-2-beta-like 1) |
| 732 | HLA-A*01:01_YSSSSPLTY | 616 | AP2D | ENSG00000008197 | Transcription factor AP-2-delta (AP2-delta) |
| | | | | | (Activating enhancer-binding protein 2-delta) |
| | | | | | (Transcription factor AP-2-beta-like 1) |
| 733 | HLA-B*51:01_DAPPAILTF | 617 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 734 | HLA-B*18:01_EEIAFSTY | 618 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 735 | HLA-A*26:01_EINPHMSSY | 619 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 736 | HLA-A*11:01_SSYVPIFEK | 620 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 737 | HLA-A*11:01_STIDEYVHR | 621 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 738 | HLA-A*11:01_STMGGFGVGK | 622 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 739 | HLA-A*02:07_TIDEYVHRI | 623 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 740 | HLA-A*30:02_TLNNNIAKAGY | 624 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 741 | HLA-A*11:01_AISITPVHK | 625 | CT55 | ENSG00000169551 | Cancer/testis antigen 55 (Tumor antigen BJ- |
| | | | | | HCC-20) |
| 742 | HLA-B*44:03_AESPLEVPQSF | 626 | MAGBG | ENSG00000189023 | Melanoma-associated antigen B16 (MAGE-B16 |
| | | | | | antigen) |
| 743 | HLA-A*02:07_ALDQKVAFL | 627 | MAGBG | ENSG00000189023 | Melanoma-associated antigen B16 (MAGE-B16 |
| | | | | | antigen) |
| 744 | HLA-A*26:01_EVLNLTGVY | 628 | MAGBG | ENSG00000189023 | Melanoma-associated antigen B16 (MAGE-B16 |
| | | | | | antigen) |
| 745 | HLA-A*29:02_LFIKLGLTY | 629 | MAGBG | ENSG00000189023 | Melanoma-associated antigen B16 (MAGE-B16 |
| | | | | | antigen) |
| 746 | HLA-A*29:02_SLAEQILAKY | 630 | FOXR2 | ENSG00000189299 | Forkhead box protein R2 (Forkhead box protein |
| | | | | | N6) |
| 747 | HLA-A*11:01_SSSSEQSPLQK | 631 | FOXR2 | ENSG00000189299 | Forkhead box protein R2 (Forkhead box protein |
| | | | | | N6) |
| 748 | HLA-B*51:01_LPNTSIHGI | 632 | NMUR2 | ENSG00000132911 | Neuromedin-U receptor 2 (NMU-R2)(G-protein |
| | | | | | coupled receptor FM-4)(G-protein coupled |
| | | | | | receptor TGR-1) |
| 749 | HLA-B*51:01_YPNSPVQVI | 633 | DAZL | ENSG00000092345 | Deleted in azoospermia-like (DAZ homolog) |
| | | | | | (DAZ-like autosomal)(Deleted in azoospermia- |
| | | | | | like 1)(SPGY-like-autosomal) |
| 750 | HLA-B*35:01_HAAGFGPEL | 634 | MBOA4 | ENSG00000177669 | Ghrelin O-acyltransferase (EC 2.3.1.-) |
| | | | | | (Membrane-bound O-acyltransferase domain- |
| | | | | | containing protein 4)(O-acyltransferase domain- |
| | | | | | containing protein 4) |
| 751 | HLA-A*29:02_GVLILLVRY | 635 | ADAM7 | ENSG00000069206 | Disintegrin and metalloproteinase domain- |
| | | | | | containing protein 7 (ADAM 7)(Sperm |
| | | | | | maturation-related glycoprotein GP-83) |
| 752 | HLA-A*02:01_GLYGINEDIFL | 636 | LDH6A | ENSG00000166800 | L-lactate dehydrogenase A-like 6A (EC 1.1.1.27) |
| 753 | HLA-B*46:01_LMIPNITQY | 637 | LDH6A | ENSG00000166800 | L-lactate dehydrogenase A-like 6A (EC 1.1.1.27) |
| 754 | HLA-A*11:01_SVADLTESILK | 638 | LDH6A | ENSG00000166800 | L-lactate dehydrogenase A-like 6A (EC 1.1.1.27) |
| 755 | HLA-A*02:01_TLWEIQKELKL | 639 | LDH6A | ENSG00000166800 | L-lactate dehydrogenase A-like 6A (EC 1.1.1.27) |
| 756 | HLA-B*35:01_MPHEVTHSM | 640 | V9GZ31 | ENSG00000177414 | Ubiquitin-conjugating enzyme E2 U (Fragment) |
| 757 | HLA-A*03:01_ATFQTTLPTLK | 641 | GDPD4 | ENSG00000178795 | Glycerophosphodiester phosphodiesterase |
| | | | | | domain-containing protein 4 (EC 3.1.-.-) |
| | | | | | (Glycerophosphodiester phosphodiesterase 6) |
| | | | | | (UgPQ) |
| 758 | HLA-A*11:01_ATFQTTLPTLK | 641 | GDPD4 | ENSG00000178795 | Glycerophosphodiester phosphodiesterase |
| | | | | | domain-containing protein 4 (EC 3.1.-.-) |
| | | | | | (Glycerophosphodiester phosphodiesterase 6) |
| | | | | | (UgPQ) |
| 759 | HLA-B*18:01_LETDIHLSY | 642 | GDPD4 | ENSG00000178795 | Glycerophosphodiester phosphodiesterase |
| | | | | | domain-containing protein 4 (EC 3.1.-.-) |
| | | | | | (Glycerophosphodiester phosphodiesterase 6) |
| | | | | | (UgPQ) |
| 760 | HLA-A*02:07_VLDQNRSTL | 643 | PERL | ENSG00000167419 | Lactoperoxidase (LPO)(EC 1.11.1.7)(Salivary |
| | | | | | peroxidase)(SPO) |
| 761 | HLA-B*44:03_QEGSSGMELSW | 644 | TEX19 | ENSG00000182459 | Testis-expressed protein 19 |
| 762 | HLA-C*16:01_IEAELHISY | 254 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 763 | HLA-B*51:01_VPLSTVNRV | 645 | TIFAB | ENSG00000255833 | TRAF-interacting protein with FHA domain- |
| | | | | | containing protein B (TWA-like protein) |
| 764 | HLA-B*35:01_AAAAAGLAY | 646 | BHE23 | ENSG00000125533 | Class E basic helix-loop-helix protein 23 |
| | | | | | (bHLHe23)(Class B basic helix-loop-helix |
| | | | | | protein 4)(bHLHb4) |
| 765 | HLA-B*51:01_IPYAHSPSV | 647 | BHE23 | ENSG0000012553 3 | Class E basic helix-loop-helix protein 23 |
| | | | | | (bHLHe23)(Class B basic helix-loop-helix |
| | | | | | protein 4)(bHLHb4) |
| 766 | HLA-B*18:01_EEFNVLEM | 648 | FATE1 | ENSG00000147378 | Fetal and adult testis-expressed transcript protein |
| | | | | | (Cancer/testis antigen 43)(CT43)(Tumor antigen |
| | | | | | BJ-HCC-2) |
| 767 | HLA-B*51:01_LPPGGIPGI | 649 | ZFP42 | ENSG00000179059 | Zinc finger protein 42 homolog (Zfp-42) |
| | | | | | (Reduced expression protein 1)(REX-1)(hREX- |
| | | | | | 1)(Zinc finger protein 754) |
| 768 | HLA-C*02:02_KEADPTGHSY | 650 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 769 | HLA-A*01:01_HTEDKPYKY | 462 | ZNF99 | ENSG00000213973 | Zinc finger protein 99 |
| 770 | HLA-A*02:01_ILYDLKIAL | 651 | ZNF99 | ENSG00000213973 | Zinc finger protein 99 |
| 771 | HLA-A*03:01_KVFNNSSTLMK | 652 | ZNF99 | ENSG00000213973 | Zinc finger protein 99 |
| 772 | HLA-C*02:02_AEQQPQPQF | 653 | MORC1 | ENSG00000114487 | MORC family CW-type zinc finger protein 1 |
| | | | | | (Cancer/testis antigen 33)(CT33) |
| 773 | HLA-B51:01_IPLGTMSTI | 654 | MORC1 | ENSG00000114487 | MORC family CW-type zinc finger protein 1 |
| | | | | | (Cancer/testis antigen 33)(CT33) |
| 774 | HLA-B*18:01_QEFLNVQEY | 655 | MORC1 | ENSG00000114487 | MORC family CW-type zinc finger protein 1 |
| | | | | | (Cancer/testis antigen 33)(CT33) |
| 775 | HLA-B*44:03_QEFLNVQEY | 655 | MORC1 | ENSG00000114487 | MORC family CW-type zinc finger protein 1 |
| | | | | | (Cancer/testis antigen 33)(CT33) |
| 776 | HLA-B*18:01_TEAELMQQF | 656 | MORC1 | ENSG00000114487 | MORC family CW-type zinc finger protein 1 |
| | | | | | (Cancer/testis antigen 33)(CT33) |
| 777 | HLA-B*44:03_TEAELMQQF | 656 | MORC1 | ENSG00000114487 | MORC family CW-type zinc finger protein 1 |
| | | | | | (Cancer/testis antigen 33)(CT33) |
| 778 | HLA-C*05:01_VTDDPQKF | 657 | MORC1 | ENSG00000114487 | MORC family CW-type zinc finger protein 1 |
| | | | | | (Cancer/testis antigen 33)(CT33) |
| 779 | HLA-A*29:02_SLPDKVFIKY | 658 | RXFP2 | ENSG00000133105 | Relaxin receptor 2 (G-protein coupled receptor |
| | | | | | 106)(G-protein coupled receptor affecting |
| | | | | | testicular descent)(Leucine-rich repeat- |
| | | | | | containing G-protein coupled receptor 8)(Relaxin |
| | | | | | family peptide receptor 2) |
| 780 | HLA-A*26:01_EVAERGTAY | 659 | MMP20 | ENSG00000137674 | Matrix metalloproteinase-20 (MMP-20)(EC |
| | | | | | 3.4.24.-)(Enamel metalloproteinase) |
| | | | | | (Enamelysin) |
| 781 | HLA-A*29:02_FFSGPKTYKY | 660 | MMP20 | ENSG00000137674 | Matrix metalloproteinase-20 (MMP-20)(EC |
| | | | | | 3.4.24.-)(Enamel metalloproteinase) |
| | | | | | (Enamelysin) |
| 782 | HLA-B*44:03_GEADIMISF | 661 | MMP20 | ENSG00000137674 | Matrix metalloproteinase-20 (MMP-20)(EC |
| | | | | | 3.4.24.-)(Enamel metalloproteinase) |
| | | | | | (Enamelysin) |
| 783 | HLA-C*02:02_GEADIMISF | 661 | MMP20 | ENSG00000137674 | Matrix metalloproteinase-20 (MMP-20)(EC |
| | | | | | 3.4.24.-)(Enamel metalloproteinase) |
| | | | | | (Enamelysin) |
| 784 | HLA-B*35:01_LPASGLAVF | 662 | MMP20 | ENSG00000137674 | Matrix metalloproteinase-20 (MMP-20)(EC |
| | | | | | 3.4.24.-)(Enamel metalloproteinase) |
| | | | | | (Enamelysin) |
| 785 | HLA-A*11:01_SSFDAVTMLGK | 663 | MMP20 | ENSG00000137674 | Matrix metalloproteinase-20 (MMP-20)(EC |
| | | | | | 3.4.24.-)(Enamel metalloproteinase) |
| | | | | | (Enamelysin) |
| 786 | HLA-A*01:01_STDPSALMY | 664 | MMP20 | ENSG00000137674 | Matrix metalloproteinase-20 (MMP-20)(EC |
| | | | | | 3.4.24.-)(Enamel metalloproteinase) |
| | | | | | (Enamelysin) |
| 787 | HLA-A*29:02_YFFSGPKTYKY | 665 | MMP20 | ENSG00000137674 | Matrix metalloproteinase-20 (MMP-20)(EC |
| | | | | | 3.4.24.-)(Enamel metalloproteinase) |
| | | | | | (Enamelysin) |
| 788 | HLA-A*24:02_IYSGNSYYF | 666 | CNTP5 | ENSG00000155052 | Contactin-associated protein-like 5 (Cell |
| | | | | | recognition molecule Caspr5) |
| 789 | HLA-A*33:01_DMQAVETYR | 667 | PDCL2 | ENSG00000163440 | Phosducin-like protein 2 |
| 790 | HLA-A*31:01_ASWARIAAR | 668 | DPPA2 | ENSG00000163530 | Developmental pluripotency-associated protein 2 |
| | | | | | (Pluripotent embryonic stem cell-related gene 1 |
| | | | | | protein) |
| 791 | HLA-B*44:03_KEDNPSGHTY | 669 | MAGB1 | ENSG00000214107 | Melanoma-associated antigen B1 (Cancer/testis |
| | | | | | antigen 3.1)(CT3.1)(DSS-AHC critical interval |
| | | | | | MAGE superfamily 10)(DAM10)(MAGE-Bl |
| | | | | | antigen)(MAGE-XP antigen) |
| 792 | HLA-A*03:01_TVAVTQMNK | 242 | ACTL8 | ENSG00000117148 | Actin-like protein 8 (Cancer/testis antigen 57) |
| | | | | | (CT57) |
| 793 | HLA-A*31:01_AVRGSDTLWYR | 670 | RNF17 | ENSG00000132972 | RING finger protein 17 (Tudor domain-containing protein 4) |
| 794 | HLA-B*08:01_YQKEKNVSI | 671 | I22R2 | ENSG00000164485 | Interleukin-22 receptor subunit alpha-2 (IL-22 |
| | | | | | receptor subunit alpha-2)(IL-22R-alpha-2)(IL- |
| | | | | | 22RA2)(Cytokine receptor class-II member 10) |
| | | | | | (Cytokine receptor family 2 member 10)(CRF2- |
| | | | | | 10)(Cytokine receptor family type 2, soluble 1) |
| | | | | | (CRF2-S1)(Interleukin-22-binding protein)(IL- |
| | | | | | 22BP)(IL22BP)(ZcytoR16) |
| 795 | HLA-C*04:01_FFDSIIAEL | 672 | A0A1B0GVHE | ENSG00000226792 | Long intergenic non-protein coding RNA 371 |
| 796 | HLA-B*27:02_VDQIALPNLK | 673 | DYTN | ENSG00000232125 | Dystrotelin |
| 797 | HLA-A*33:01_DTFRSISTR | 674 | WNT8B | ENSG00000075290 | Protein Wnt-gb |
| 798 | HLA-B*27:02_GRGAIADTF | 675 | WNT8B | ENSG00000075290 | Protein Wnt-8b |
| 799 | HLA-B*44:03_AESEGTKAVL | 676 | H2BWT | ENSG00000123569 | Histone H2B type W-T (H2B histone family |
| | | | | | member W testis-specific) |
| 800 | HLA-C*02:02_AESEGTKAVL | 676 | H2BWT | ENSG00000123569 | Histone H2B type W-T (H2B histone family |
| | | | | | member W testis-specific) |
| 801 | HLA-A*03:01_SLYAIQQQRK | 677 | H2BWT | ENSG00000123569 | Histone H2B type W-T (H2B histone family |
| | | | | | member W testis-specific) |
| 802 | HLA-B*18:01_DEAGMLSYF | 678 | APOL5 | ENSG00000128313 | Apolipoprotein L5 (Apolipoprotein L-V)(ApoL- |
| | | | | | V) |
| 803 | HLA-B*18:01_DEAGMLSY | 679 | APOL5 | ENSG00000128313 | Apolipoprotein L5 (Apolipoprotein L-V)(ApoL- |
| | | | | | V) |
| 804 | HLA-B*44:03_EEEKLFLSY | 680 | APOL5 | ENSG00000128313 | Apolipoprotein L5 (Apolipoprotein L-V)(ApoL- |
| | | | | | V) |
| 805 | HLA-B*08:01_ELLTKTSL | 681 | APOL5 | ENSG00000128313 | Apolipoprotein L5 (Apolipoprotein L-V)(ApoL- |
| | | | | | V) |
| 806 | HLA-A*01:01_HSDEAGMLSY | 682 | APOL5 | ENSG00000128313 | Apolipoprotein L5 (Apolipoprotein L-V)(ApoL- |
| | | | | | V) |
| 807 | HLA-A*31:01_IVTNVLENR | 683 | APOL5 | ENSG00000128313 | Apolipoprotein L5 (Apolipoprotein L-V)(ApoL- |
| | | | | | V) |
| 808 | HLA-A*33:01_IVTNVLENR | 683 | APOL5 | ENSG00000128313 | Apolipoprotein L5 (Apolipoprotein L-V)(ApoL- |
| | | | | | V) |
| 809 | HLA-A*30:02_QGIKDLHAY | 684 | APOL5 | ENSG00000128313 | Apolipoprotein L5 (Apolipoprotein L-V)(ApoL- |
| | | | | | V) |
| 810 | HLA-B*44:03_SEEEKLFLSY | 685 | APOL5 | ENSG00000128313 | Apolipoprotein L5 (Apolipoprotein L-V)(ApoL- |
| | | | | | V) |
| 811 | HLA-B*18:01_SEIEAAGF | 686 | APOL5 | ENSG00000128313 | Apolipoprotein L5 (Apolipoprotein L-V)(ApoL- |
| | | | | | V) |
| 812 | HLA-A*11:01_ASVLIFANK | 687 | K7EM39 | ENSG00000141748 | Putative ADP-ribosylation factor-like protein 5C |
| | | | | | (Fragment) |
| 813 | HLA-B*44:03_EEQSLQKLY | 688 | SPT21 | ENSG00000187144 | Spermatogenesis-associated protein 21 |
| 814 | HLA-A*01:01_QSSERTLSY | 689 | SPT21 | ENSG00000187144 | Spermatogenesis-associated protein 21 |
| 815 | HLA-A*11:01_ITQDLVQEK | 690 | MAGB1 | ENSG00000214107 | Melanoma-associated antigen B1 (Cancer/testis |
| | | | | | antigen 3.1)(CT3.1)(DSS-AHC critical interval |
| | | | | | MAGE superfamily 10)(DAM10)(MAGE-Bi |
| | | | | | antigen)(MAGE-XP antigen) |
| 816 | HLA-A*29:02_FVADSFPFY | 691 | FOXI3 | ENSG00000214336 | Forkhead box protein I3 |
| 817 | HLA-B*18:01_DEGEHLVF | 692 | ADAM7 | ENSG00000069206 | Disintegrin and metalloproteinase domain- |
| | | | | | containing protein 7 (ADAM 7)(Sperm |
| | | | | | maturation-related glycoprotein GP-83) |
| 818 | HLA-A*26:01_EVATAVNTR | 693 | RNF17 | ENSG00000132972 | RING finger protein 17 (Tudor domain-containing protein 4) |
| 819 | HLA-A*02:07_ILPLRFVEL | 694 | F71F1 | ENSG00000135248 | Protein FAM71F1 (Protein FAM137A)(Testis |
| | | | | | development protein NYD-SP18) |
| 820 | HLA-A*02:01_TVTEKIYYL | 695 | F71F1 | ENSG00000135248 | Protein FAM71F1 (Protein FAM137A)(Testis |
| | | | | | development protein NYD-SP18) |
| 821 | HLA-A*26:01_EVTNHNIRLF | 696 | TRI43 | ENSG00000144015 | Tripartite motif-containing protein 43 |
| 822 | HLA-A*24:02_EYQEIFQQL | 697 | TRI43 | ENSG00000144015 | Tripartite motif-containing protein 43 |
| 823 | HLA-A*29:02_LTFPVRPFFY | 698 | TRI43 | ENSG00000144015 | Tripartite motif-containing protein 43 |
| 824 | HLA-B*35:01_MPQPVNPEL | 699 | TRI43 | ENSG00000144015 | Tripartite motif-containing protein 43 |
| 825 | HLA-A*24:02_NYLVDPVTI | 700 | TRI43 | ENSG00000144015 | Tripartite motif-containing protein 43 |
| 826 | HLA-A*11:01_SVSFLNVTK | 701 | TRI43 | ENSG00000144015 | Tripartite motif-containing protein 43 |
| 827 | HLA-B*44:03_GEYPDYQQW | 604 | CRGC | ENSG00000163254 | Gamma-crystallin C (Gamma-C-crystallin) |
| | | | | | (Gamma-crystallin 2-1)(Gamma-crystallin 3) |
| 828 | HLA-C*02:02_GEYPDYQQW | 604 | CRGC | ENSG00000163254 | Gamma-crystallin C (Gamma-C-crystallin) |
| | | | | | (Gamma-crystallin 2-1)(Gamma-crystallin 3) |
| 829 | HLA-B*44:03_EEITQGNTL | 702 | CC049 | ENSG00000163632 | Putative uncharacterized protein C3orf49 |
| 830 | HLA-A*31:01_ITQGNTLLRAR | 703 | CC049 | ENSG00000163632 | Putative uncharacterized protein C3orf49 |
| 831 | HLA-B*35:01_LPEPFKIAY | 704 | CC049 | ENSG00000163632 | Putative uncharacterized protein C3orf49 |
| 832 | HLA-A*02:07_QVDDLIETV | 705 | CC049 | ENSG00000163632 | Putative uncharacterized protein C3orf49 |
| 833 | HLA-A*11:01_VTSLPSGLQK | 706 | CC049 | ENSG00000163632 | Putative uncharacterized protein C3orf49 |
| 834 | HLA-A*29:02_YLPEPFKIAY | 707 | CC049 | ENSG00000163632 | Putative uncharacterized protein C3orf49 |
| 835 | HLA-A*29:02_EVVGVVYVY | 168 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 836 | HLA-B*35:01_TVQGVVTSF | 708 | CT55 | ENSG00000169551 | Cancer/testis antigen 55 (Tumor antigen BJ- |
| | | | | | HCC-20) |
| 837 | HLA-A*11:01_ASLQDILSH | 709 | S22AD | ENSG00000172940 | Solute carrier family 22 member 13 (Organic |
| | | | | | cation transporter-like 3)(ORCTL-3) |
| 838 | HLA-A*01:01_YTESRSFNY | 710 | EP3B | ENSG00000181552 | Epididymal secretoiy protein E3-beta (Human |
| | | | | | epididymis-specific protein 3-beta)(HE3-beta) |
| 839 | HLA-A*02:07_LLDDIMAEV | 711 | TSPY1 | ENSG00000258992 | Testis-specific Y-encoded protein 1 (Cancer/testis |
| | | | | | antigen 78)(CT78) |
| 840 | HLA-A*02:01_SLDEALQRV | 540 | RNF17 | ENSG00000132972 | RING finger protein 17 (Tudor domain- |
| | | | | | containing protein 4) |
| 841 | HLA-B*51:01_DAVEALYKV | 712 | CBPA5 | ENSG00000158525 | Carboxypeptidase A5 (EC 3.4.17.-) |
| 842 | HLA-A*01:01_YLESHGLAY | 713 | CBPA5 | ENSG00000158525 | Carboxypeptidase A5 (EC 3.4.17.-) |
| 843 | HLA-A*02:07_LLDDIMAEV | 711 | C9JPU3 | ENSG00000168757 | Testis-specific Y-encoded protein 2 |
| 844 | HLA-A*30:02_AVQTSYTSY | 714 | UROL1 | ENSG00000177398 | Uromodulin-like 1 (Olfactorin) |
| 845 | HLA-B*44:03_NEVVVSFKW | 715 | LY6L | ENSG00000261667 | Lymphocyte antigen 6L (Lymphocyte antigen 6 |
| | | | | | complex locus protein L) |
| 846 | HLA-A*26:01_DAPPAILTF | 617 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 847 | HLA-A*30:02_NVNGQTISLY | 716 | LMTD1 | ENSG00000152936 | Lamin tail domain-containing protein 1 |
| | | | | | (Intermediate filament tail domain-containing |
| | | | | | protein 1) |
| 848 | HLA-A*02:01_SLDASPFSV | 717 | LMTD1 | ENSG00000152936 | Lamin tail domain-containing protein 1 |
| | | | | | (Intermediate filament tail domain-containing |
| | | | | | protein 1) |
| 849 | HLA-A*11:01_STATITKEK | 718 | LMTD1 | ENSG00000152936 | Lamin tail domain-containing protein 1 |
| | | | | | (Intermediate filament tail domain-containing |
| | | | | | protein 1) |
| 850 | HLA-A*11:01_STTGQLTSK | 719 | LMTD1 | ENSG00000152936 | Lamin tail domain-containing protein 1 |
| | | | | | (Intermediate filament tail domain-containing |
| | | | | | protein 1) |
| 851 | HLA-B*51:01_IPLTIISI | 720 | NPSR1 | ENSG00000187258 | Neuropeptide S receptor (G-protein coupled |
| | | | | | receptor 154)(G-protein coupled receptor |
| | | | | | PGR14)(G-protein coupled receptor for asthma |
| | | | | | susceptibility) |
| 852 | HLA-B*51:01_LPALEPVI | 721 | PRS48 | ENSG00000189099 | Serine protease 48 (EC 3.4.21.-)(Epidermis- |
| | | | | | specific serine protease-like protein) |
| 853 | HLA-B*44:03_SEGTKVPAW | 722 | CLC6A | ENSG00000205846 | C-type lectin domain family 6 member A (C-type |
| | | | | | lectin superfamily member 10)(Dendritic cell- |
| | | | | | associated C-type lectin 2)(DC-associated C-type |
| | | | | | lectin 2)(Dectin-2) |
| 854 | HLA-A*30:02_YQGSIVHEY | 723 | ADAM7 | ENSG00000069206 | Disintegrin and metalloproteinase domain- |
| | | | | | containing protein 7 (ADAM 7)(Sperm |
| | | | | | maturation-related glycoprotein GP-83) |
| 855 | HLA-B*08:01_HMAHKVNSL | 724 | MROH9 | ENSG00000117501 | Maestro heat-like repeat-containing protein |
| | | | | | family member 9 |
| 856 | HLA-A*02:07_IVDAIYRQL | 725 | MROH9 | ENSG00000117501 | Maestro heat-like repeat-containing protein |
| | | | | | family member 9 |
| 857 | HLA-A*30:02_KVNSLLDAY | 726 | MROH9 | ENSG00000117501 | Maestro heat-like repeat-containing protein |
| | | | | | family member 9 |
| 858 | HLA-B*08:01_NPKTKSSL | 727 | MROH9 | ENSG00000117501 | Maestro heat-like repeat-containing protein |
| | | | | | family member 9 |
| 859 | HLA-B*18:01_SESLAAVF | 728 | NMUR2 | ENSG00000132911 | Neuromedin-U receptor 2 (NMU-R2)(G-protein |
| | | | | | coupled receptor FM-4)(G-protein coupled |
| | | | | | receptor TGR-1) |
| 860 | HLA-B*51:01_IPYLQTVSV | 729 | OX2R | ENSG00000137252 | Orexin receptor type 2 (Ox-2-R)(Ox2-R)(Ox2R) |
| | | | | | (Hypocretin receptor type 2) |
| 861 | HLA-A*02:01_SLADVLVTI | 730 | OX2R | ENSG00000137252 | Orexin receptor type 2 (Ox-2-R)(Ox2-R)(Ox2R) |
| | | | | | (Hypocretin receptor type 2) |
| 862 | HLA-A*26:01_EVITTVYGY | 731 | ZAN | ENSG00000146839 | Zonadhesin |
| 863 | HLA-A*02:01_FLQEVITTV | 732 | ZAN | ENSG00000146839 | Zonadhesin |
| 864 | HLA-A*30:02_GQSPGAALHIY | 733 | ZAN | ENSG00000146839 | Zonadhesin |
| 865 | HLA-B*18:01_LEIEIPTTY | 734 | ZAN | ENSG00000146839 | Zonadhesin |
| 866 | HLA-A*30:02_SGHGVSSRY | 735 | ZAN | ENSG00000146839 | Zonadhesin |
| 867 | HLA-B*18:01_VEVTVPSSY | 736 | ZAN | ENSG00000146839 | Zonadhesin |
| 868 | HLA-A*02:01_GLMAVNQEV | 737 | PERL | ENSG00000167419 | Lactoperoxidase (LPO)(EC 1.11.1.7)(Salivary |
| | | | | | peroxidase)(SPO) |
| 869 | HLA-B*44:03_EEPLSVTASY | 738 | VCX3 | ENSG00000169059 | Variable charge X-linked protein 3 (Variable |
| | | | | | charge protein on X with eight repeats)(VCX-8r) |
| | | | | | (Variably charged protein X-A)(VCX-A) |
| 870 | HLA-B*18:01_EESPFLVAV | 739 | HDGL1 | ENSG00000112273 | Hepatoma-derived growth factor-like protein 1 |
| | | | | | (PWWP domain-containing protein 1) |
| 871 | HLA-B*18:01_DEMGVVGY | 740 | OTOR | ENSG00000125879 | Otoraplin (Fibrocyte-derived protein)(Melanoma |
| | | | | | inhibitory activity-like protein) |
| 872 | HLA-B*44:03_HEAFGGINW | 741 | APOL5 | ENSG00000128313 | Apolipoprotein L5 (Apolipoprotein L-V)(ApoL- |
| | | | | | V) |
| 873 | HLA-A*29:02_FFLSMVNNY | 742 | RXFP2 | ENSG00000133105 | Relaxin receptor 2 (G-protein coupled receptor |
| | | | | | 106)(G-protein coupled receptor affecting |
| | | | | | testicular descent)(Leucine-rich repeat- |
| | | | | | containing G-protein coupled receptor 8)(Relaxin |
| | | | | | family peptide receptor 2) |
| 874 | HLA-A*29:02_IFSQHTFKY | 743 | IFNK | ENSG00000147896 | Interferon kappa (IFN-kappa) |
| 875 | HLA-C*02:02_QEINTKSAF | 744 | GPC6A | ENSG00000173612 | G-protein coupled receptor family C group 6 |
| | | | | | member A (hGPRC6A)(G-protein coupled |
| | | | | | receptor GPCR33)(hGPCR33) |
| 876 | HLA-B*44:03_SESSTILVVRY | 316 | SPNXC | ENSG00000198573 | Sperm protein associated with the nucleus on the |
| | | | | | X chromosome C (Cancer/testis antigen 11.3) |
| | | | | | (CT11.3)(Cancer/testis-associated protein |
| | | | | | CTp11)(Nuclear-associated protein SPAN-Xe) |
| | | | | | (SPANX-C)(SPANX family member C) |
| 877 | HLA-A*24:02_SYLGISAVSEF | 745 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 878 | HLA-A*11:01_ATQRDLIATQK | 746 | LUZP4 | ENSG00000102021 | Leucine zipper protein 4 (Cancer/testis antigen |
| | | | | | 28)(CT-28)(CT28)(Tumor antigen HOM-TES- |
| | | | | | 85) |
| 879 | HLA-B*46:01_AVKKGSTAY | 747 | PSA7L | ENSG00000154611 | Proteasome subunit alpha type-7-like (EC |
| | | | | | 3.4.25.1) |
| 880 | HLA-A*33:01_DARVVINR | 748 | PSA7L | ENSG00000154611 | Proteasome subunit alpha type-7-like (EC |
| | | | | | 3.4.25.1) |
| 881 | HLA-B*51:01_TAYHVSLIV | 749 | PSA7L | ENSG00000154611 | Proteasome subunit alpha type-7-like (EC |
| | | | | | 3.4.25.1) |
| 882 | HLA-B*18:01_VEDPVTVEY | 750 | PSA7L | ENSG00000154611 | Proteasome subunit alpha type-7-like (EC |
| | | | | | 3.4.25.1) |
| 883 | HLA-B*46:01_VVIEQSSSL | 119 | TERT | ENSG00000164362 | Telomerase reverse transcriptase (EC 2.7.7.49) |
| | | | | | (HEST2)(Telomerase catalytic subunit) |
| | | | | | (Telomerase-associated protein 2)(TP2) |
| 884 | HLA-B*44:02_AEARPVPHW | 156 | ACHA9 | ENSG00000174343 | Neuronal acetylcholine receptor subunit alpha-9 |
| | | | | | (Nicotinic acetylcholine receptor subunit alpha-9) |
| | | | | | (NACHR alpha-9) |
| 885 | HLA-C*05:01_NADLQSEFL | 751 | V9GYR9 | ENSG00000225362 | Cancer/testis antigen 62 (Fragment) |
| 886 | HLA-B*35:01_HAEDGTILF | 752 | PIWL1 | ENSG00000125207 | Piwi-like protein 1 (EC 3.1.26.-) |
| 887 | HLA-A*30:02_KTGPSRSSY | 753 | RNF17 | ENSG00000132972 | RING finger protein 17 (Tudor domain- |
| | | | | | containing protein 4) |
| 888 | HLA-A*03:01_AVYNSPQFK | 561 | FBX39 | ENSG00000177294 | F-box only protein 39 |
| 889 | HLA-A*11:01_ASAVQLPEK | 754 | CB091 | ENSG00000205086 | Uncharacterized protein C2orf91 |
| 890 | HLA-A*11:01_ATPSADFLLK | 755 | E9PHT4 | ENSG00000206199 | Protein ANKUB 1 |
| 891 | HLA-A*11:01_AVASAFKEK | 756 | E9PHT4 | ENSG00000206199 | Protein ANKUB 1 |
| 892 | HLA-A*29:02_GYSHPSFFY | 757 | E9PHT4 | ENSG00000206199 | Protein ANKUB 1 |
| 893 | HLA-A*26:01_ETTDIKGLF | 758 | CD051 | ENSG00000237136 | Uncharacterized protein C4orf51 |
| 894 | HLA-A*30:02_AQLGGLSDGY | 759 | HXB1 | ENSG00000120094 | Homeobox protein Hox-B1 (Homeobox protein |
| | | | | | Hox-2I) |
| 895 | HLA-B*44:03_TELEKEFHF | 760 | HXB1 | ENSG00000120094 | Homeobox protein Hox-B1 (Homeobox protein |
| | | | | | Hox-2I) |
| 896 | HLA-B*44:03_EEDVWVIIITLY | 761 | PDCL2 | ENSG00000163440 | Phosducin-like protein 2 |
| 897 | HLA-A*03:01_ALQLVPGSPK | 762 | WED13 | ENSG00000168634 | WAP four-disulfide core domain protein 13 |
| 898 | HLA-B*35:01_MAGASIPAM | 763 | SAGE1 | ENSG00000181433 | Sarcoma antigen 1 (Cancer/testis antigen 14) |
| | | | | | (CT14) |
| 899 | HLA-B*18:01_FEAGTSVTY | 764 | CNTP5 | ENSG00000155052 | Contactin-associated protein-like 5 (Cell |
| | | | | | recognition molecule Caspr5) |
| 900 | HLA-A*30:02_KAFDDIATY | 406 | SSX1 | ENSG00000126752 | Protein SSX1 (Cancer/testis antigen 5.1)(CT5.1) |
| | | | | | (Synovial sarcoma, X breakpoint 1) |
| 901 | HLA-A*02:01_SQDSFIPGV | 765 | CER1 | ENSG00000147869 | Cerbems (Cerbems-related protein)(DAN |
| | | | | | domain family member 4) |
| 902 | HLA-B*51:01_MPYAHGPSV | 766 | OLIG3 | ENSG00000177468 | Oligodendrocyte transcription factor 3 (01ig03) |
| | | | | | (Class B basic helix-loop-helix protein 7) |
| | | | | | (bHLHb7)(Class E basic helix-loop-helix protein |
| | | | | | 20)(bHLHe20) |
| 903 | HLA-B*44:03_SEQDLQQLRL | 767 | OLIG3 | ENSG00000177468 | Oligodendrocyte transcription factor 3 (01ig03) |
| | | | | | (Class B basic helix-loop-helix protein 7) |
| | | | | | (bHLHb7)(Class E basic helix-loop-helix protein |
| | | | | | 20)(bHLHe20) |
| 904 | HLA-C*01:02_SSPLSAASL | 768 | OLIG3 | ENSG00000177468 | Oligodendrocyte transcription factor 3 (01ig03) |
| | | | | | (Class B basic helix-loop-helix protein 7) |
| | | | | | (bHLHb7)(Class E basic helix-loop-helix protein |
| | | | | | 20)(bHLHe20) |
| 905 | HLA-A*11:01_STQGDMMQK | 769 | OLIG3 | ENSG00000177468 | Oligodendrocyte transcription factor 3 (01ig03) |
| | | | | | (Class B basic helix-loop-helix protein 7) |
| | | | | | (bHLHb7)(Class E basic helix-loop-helix protein |
| | | | | | 20)(bHLHe20) |
| 906 | HLA-A*11:01_TTADVALLK | 770 | PRS48 | ENSG00000189099 | Serine protease 48 (EC 3.4.21.-)(Epidermis- |
| | | | | | specific serine protease-like protein) |
| 907 | HLA-B*35:01_NAFKFSSTF | 771 | ZN729 | ENSG00000196350 | Zinc finger protein 729 |
| 908 | HLA-B*44:03_AETDNLDHY | 772 | GLYL3 | ENSG00000203972 | Glycine N-acyltransferase-like protein 3 (EC |
| | | | | | 2.3.1.-) |
| 909 | HLA-A*30:02_TTHIANHSY | 773 | Q5JUY5 | ENSG00000117400 | Thrombopoietin receptor |
| 910 | HLA-A*11:01_ATVEEDFQPFR | 774 | PRD14 | ENSG00000147596 | PR domain zinc finger protein 14 (EC 2.1.1.-) |
| | | | | | (PR domain-containing protein 14) |
| 911 | HLA-B*44:03_AEFPNSFVTL | 775 | ADA18 | ENSG00000168619 | Disintegrin and metalloproteinase domain- |
| | | | | | containing protein 18 (ADAM 18) |
| | | | | | (Transmembrane metalloproteinase-like, |
| | | | | | disintegrin-like, and cysteine-rich protein III) |
| | | | | | (tMDC III) |
| 912 | HLA-A*29:02_FLPQNFLVY | 776 | ADA18 | ENSG00000168619 | Disintegrin and metalloproteinase domain- |
| | | | | | containing protein 18 (ADAM 18) |
| | | | | | (Transmembrane metalloproteinase-like, |
| | | | | | disintegrin-like, and cysteine-rich protein III) |
| | | | | | (tMDC III) |
| 913 | HLA-B*18:01_DEQQIINSF | 777 | PP2D1 | ENSG00000183977 | Protein phosphatase 2C-like domain-containing |
| | | | | | protein 1 |
| 914 | HLA-A*24:02_IYNPENVETF | 778 | PP2D1 | ENSG00000183977 | Protein phosphatase 2C-like domain-containing |
| | | | | | protein 1 |
| 915 | HLA-A*01:01_LSDSNYSKY | 779 | PP2D1 | ENSG00000183977 | Protein phosphatase 2C-like domain-containing |
| | | | | | protein 1 |
| 916 | HLA-A*11:01_STSEPNLTK | 780 | PP2D1 | ENSG00000183977 | Protein phosphatase 2C-like domain-containing |
| | | | | | protein 1 |
| 917 | HLA-A*30:02_ITQDLVQEKY | 781 | MAGB1 | ENSG00000214107 | Melanoma-associated antigen B1 (Cancer/testis |
| | | | | | antigen 3.1)(CT3.1)(DSS-AHC critical interval |
| | | | | | MAGE superfamily 10)(DAM10)(MAGE-Bl |
| | | | | | antigen)(MAGE-XP antigen) |
| 918 | HLA-A*11:01_AVMTKPKVK | 782 | PRDM7 | ENSG00000126856 | Probable histone-lysine N-methyltransferase |
| | | | | | PRDM7 (EC 2.1.1.43)(PR domain zinc finger |
| | | | | | protein 7)(PR domain-containing protein 7) |
| 919 | HLA-A*31:01_IVKVPILNR | 783 | TEX37 | ENSG00000172073 | Testis-expressed sequence 37 protein (Testis- |
| | | | | | specific conserved protein of 21 kDa) |
| 920 | HLA-B*35:01_LAVAVPVVY | 784 | NPBW1 | ENSG00000183729 | Neuropeptides B/W receptor type 1 (G-protein |
| | | | | | coupled receptor 7) |
| 921 | HLA-A*11:01_SVIDVQLGK | 785 | IRPL2 | ENSG00000189108 | X-linked interleukin-1 receptor accessory protein- |
| | | | | | like 2 (IL-1 receptor accessory protein-like 2) |
| | | | | | (IL-1-RAPL-2)(IL-1RAPL-2)(IL1RAPL-2) |
| | | | | | (IL1RAPL-2-related protein)(Interleukin-1 |
| | | | | | receptor 9)(IL-1R-9)(IL-1R9)(Three |
| | | | | | immunoglobulin domain-containing IL-1 |
| | | | | | receptor-related 1)(TIGIRR-1) |
| 922 | HLA-B*51:01_SAFGYLHSI | 786 | 1A1L2 | ENSG00000205126 | Probable inactive 1-aminocyclopropane-1- |
| | | | | | carboxylate synthase-like protein 2 (ACC |
| | | | | | synthase-like protein 2) |
| 923 | HLA-A*11:01_ASGPPAKAK | 787 | VCX3B | ENSG00000205642 | Variable charge X-linked protein 3B (Variably |
| | | | | | charged protein X-C)(VCX-C) |
| 924 | HLA-B*44:03_EEPLSVTAKY | 209 | VCX3B | ENSG00000205642 | Variable charge X-linked protein 3B (Variably |
| | | | | | charged protein X-C)(VCX-C) |
| 925 | HLA-A*24:02_AYIPKLLQLF | 788 | A0A1B0GTN1 | ENSG00000224960 | Putative SMEK homolog 3 |
| 926 | HLA-A*11:01_GTIPGPIAQR | 789 | SHP1L | ENSG00000157060 | Testicular spindle-associated protein SHCBP1L |
| | | | | | (SHC SH2 domain-binding protein 1-like protein) |
| 927 | HLA-A*03:01_KTNPSVFFVK | 790 | SHP1L | ENSG00000157060 | Testicular spindle-associated protein SHCBP1L |
| | | | | | (SHC SH2 domain-binding protein 1-like protein) |
| 928 | HLA-A*11:01_STLGGVNMK | 791 | SHP1L | ENSG00000157060 | Testicular spindle-associated protein SHCBP1L |
| | | | | | (SHC SH2 domain-binding protein 1-like protein) |
| 929 | HLA-B*51:01_VPADSFRTI | 792 | SHP1L | ENSG00000157060 | Testicular spindle-associated protein SHCBP1L |
| | | | | | (SHC SH2 domain-binding protein 1-like protein) |
| 930 | HLA-B*44:03_EEMNILAKL | 793 | TRI60 | ENSG00000176979 | Tripartite motif-containing protein 60 (RING |
| | | | | | finger protein 129)(RING finger protein 33) |
| 931 | HLA-B*44:02_QELLERQAW | 524 | ASCL4 | ENSG00000187855 | Achaete-scute homolog 4 (ASH-4)(hASH4) |
| | | | | | (Achaete-scute-like protein 4)(Class A basic |
| | | | | | helix-loop-helix protein 44)(bHLHa44) |
| 932 | HLA-A*11:01_SVQEIYNFTR | 794 | FOXR2 | ENSG00000189299 | Forkhead box protein R2 (Forkhead box protein |
| | | | | | N6) |
| 933 | HLA-A*03:01_VLNQPGILK | 795 | A0A1B0GUY1 | ENSG00000248109 | Uncharacterized protein |
| 934 | HLA-A*11:01_SIAPNIFLK | 796 | F71F1 | ENSG00000135248 | Protein FAM71F1 (Protein FAM137A)(Testis |
| | | | | | development protein NYD-SP18) |
| 935 | HLA-C*05:01_IADVDVQEV | 797 | UROL1 | ENSG00000177398 | Uromodulin-like 1 (Olfactorin) |
| 936 | HLA-A*11:01_ASGPPAPAR | 798 | NGN1 | ENSG00000181965 | Neurogenin-1 (NGN-1)(Class A basic helix-loop- |
| | | | | | helix protein 6)(bHLHa6)(Neurogenic basic- |
| | | | | | helix-loop-helix protein)(Neurogenic |
| | | | | | differentiation factor 3)(NeuroD3) |
| 937 | HLA-B*51:01_LPLYVKEI | 799 | SGCZ | ENSG00000185053 | Zeta-sarcoglycan (Zeta-SG)(ZSG1) |
| 938 | HLA-A*29:02_TFPITGLRY | 800 | DYTN | ENSG00000232125 | Dystrotelin |
| 939 | HLA-B*44:03_SEAGVYVLRF | 801 | S14L3 | ENSG00000100012 | SEC14-like protein 3 (Tocopherol-associated |
| | | | | | protein 2) |
| 940 | HLA-B*35:01_YPVTLNVLY | 802 | PSG8 | ENSG00000124467 | Pregnancy-specific beta-1-glycoprotein 8 (PS- |
| | | | | | beta-G-8)(PSBG-8)(Pregnancy-specific |
| | | | | | glycoprotein 8) |
| 941 | HLA-B*18:01_SEKISYVY | 803 | SSX1 | ENSG00000126752 | Protein SSX1 (Cancer/testis antigen 5.1)(CT5.1) |
| | | | | | (Synovial sarcoma, X breakpoint 1) |
| 942 | HLA-A*26:01_EVITSAPGAM | 804 | DPPA2 | ENSG00000163530 | Developmental pluripotency-associated protein 2 |
| | | | | | (Pluripotent embryonic stem cell-related gene 1 |
| | | | | | protein) |
| 943 | HLA-C*02:02_SENDIPSVAF | 283 | DC4L2 | ENSG00000176566 | DDB1-and CUL4-associated factor 4-like protein |
| | | | | | 2 (WD repeat-containing protein 21C) |
| 944 | HLA-C*02:02_EEMQIQKSQW | 569 | NTM2G | ENSG00000188152 | NUT family member 2G |
| 945 | HLA-A*26:01_QVINGEMQFY | 805 | NTM1B | ENSG00000203740 | Alpha N-terminal protein methyltransferase 1B |
| | | | | | (EC 2.1.1.299)(Methyltransferase-like protein |
| | | | | | 11B)(X-Pro-Lys N-terminal protein |
| | | | | | methyltransferase 1B)(NTM1B) |
| 946 | HLA-A*02:07_YLLEKIPLV | 806 | NTM1B | ENSG00000203740 | Alpha N-terminal protein methyltransferase 1B |
| | | | | | (EC 2.1.1.299)(Methyltransferase-like protein |
| | | | | | 11B)(X-Pro-Lys N-terminal protein |
| | | | | | methyltransferase 1B)(NTM1B) |
| 947 | HLA-A*11:01_ASVQGELAQLK | 807 | PTX4 | ENSG00000251692 | Pentraxin-4 |
| 948 | HLA-A*26:01_EVDPAGHSY | 808 | MAGA8 | ENSG00000156009 | Melanoma-associated antigen 8 (Cancer/testis |
| | | | | | antigen 1.8)(CT1.8)(MAGE-8 antigen) |
| 949 | HLA-A*01:01_NSDNVGYASY | 809 | V9GYJ5 | ENSG00000188611 | Neutral ceramidase (Fragment) |
| 950 | HLA-A*26:01_QVADINLMGY | 810 | V9GYJ5 | ENSG00000188611 | Neutral ceramidase (Fragment) |
| 951 | HLA-B*46:01_AAAAAGLAY | 646 | BHE23 | ENSG00000125533 | Class E basic helix-loop-helix protein 23 |
| | | | | | (bHLHe23)(Class B basic helix-loop-helix |
| | | | | | protein 4)(bHLHb4) |
| 952 | HLA-A*11:01_KTADIISEQK | 811 | RNF17 | ENSG00000132972 | RING finger protein 17 (Tudor domain-containing protein 4) |
| 953 | HLA-A*11:01_QTLNNNIAK | 812 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 954 | HLA-C*02:02_GEMPSERQY | 813 | AXDN1 | ENSG00000162779 | Axonemal dynein light chain domain-containing |
| | | | | | protein 1 |
| 955 | HLA-A*03:01_ITWDAPAITK | 271 | ACHA9 | ENSG00000174343 | Neuronal acetylcholine receptor subunit alpha-9 |
| | | | | | (Nicotinic acetylcholine receptor subunit alpha-9) |
| | | | | | (NACHR alpha-9) |
| 956 | HLA-B*51:01_LPQGSMSSI | 814 | CF010 | ENSG00000204296 | Uncharacterized protein C6orf10 |
| 957 | HLA-B*44:03_SEQSARLLDY | 815 | CF010 | ENSG00000204296 | Uncharacterized protein C6orf10 |
| 958 | HLA-A*11:01_ASFTSFNPK | 816 | M4A18 | ENSG00000214782 | Membrane-spanning 4-domains subfamily A |
| | | | | | member 18 |
| 959 | HLA-C*01:02_QYPVGTASL | 817 | M4A18 | ENSG00000214782 | Membrane-spanning 4-domains subfamily A |
| | | | | | member 18 |
| 960 | HLA-C*02:02_QEFLNVQEY | 655 | MORC1 | ENSG00000114487 | MORC family CW-type zinc finger protein 1 |
| | | | | | (Cancer/testis antigen 33)(CT33) |
| 961 | HLA-C*16:01_IEAIRAEY | 818 | TRI51 | ENSG00000124900 | Tripartite motif-containing protein 51 (SPRY |
| | | | | | domain-containing protein 5) |
| 962 | HLA-C*01:02_SSPEGTREL | 819 | CA094 | ENSG00000142698 | Uncharacterized protein C1orf94 |
| 963 | HLA-A*11:01_AVSSAALTH | 820 | WNT9B | ENSG00000158955 | Protein Wnt-9b (Protein Wnt-14b)(Protein Wnt- |
| | | | | | 15) |
| 964 | HLA-A*11:01_GTLAMILTK | 821 | OVCH1 | ENSG00000187950 | Ovochymase-1 (EC 3.4.21.-) |
| 965 | HLA-A*03:01_SVYDNVRSVGK | 822 | OVCH1 | ENSG00000187950 | Ovochymase-1 (EC 3.4.21.-) |
| 966 | HLA-A*11:01_SVYDNVRSVGK | 822 | OVCH1 | ENSG00000187950 | Ovochymase-1 (EC 3.4.21.-) |
| 967 | HLA-A*29:02_YMSPDIALLY | 823 | OVCH1 | ENSG00000187950 | Ovochymase-1 (EC 3.4.21.-) |
| 968 | HLA-B*35:01_DAWSGSNAY | 824 | CRBA1 | ENSG00000108255 | Beta-crystallin A3 [Cleaved into: Beta-crystallin |
| | | | | | A3, isoform A1, Delta4 form; Beta-crystallin A3, |
| | | | | | isoform A1, Delta7 form; Beta-crystallin A3, |
| | | | | | isoform A1, Delta8 form] |
| 969 | HLA-B*44:03_YEVLTPLKW | 825 | AMELX | ENSG00000125363 | Amelogenin, X isoform |
| 970 | HLA-B*35:01_NASNDTYLY | 826 | CSTL1 | ENSG00000125823 | Cystatin-like 1 (RCET11) |
| 971 | HLA-B*44:03_AEAITAPLF | 827 | RHXF2 | ENSG00000131721 | Rhox homeobox family member 2 (Paired-like |
| | | | | | homeobox protein PEPP-2)(Testis homeobox |
| | | | | | gene 1) |
| 972 | HLA-C*02:02_AEAITAPLF | 827 | RHXF2 | ENSG00000131721 | Rhox homeobox family member 2 (Paired-like |
| | | | | | homeobox protein PEPP-2)(Testis homeobox |
| | | | | | gene 1) |
| 973 | HLA-A*31:01_AVQIWFENR | 828 | RHXF2 | ENSG00000131721 | Rhox homeobox family member 2 (Paired-like |
| | | | | | homeobox protein PEPP-2)(Testis homeobox |
| | | | | | gene 1) |
| 974 | HLA-A*02:07_IVPSFTFPNV | 829 | RHXF2 | ENSG00000131721 | Rhox homeobox family member 2 (Paired-like |
| | | | | | homeobox protein PEPP-2)(Testis homeobox |
| | | | | | gene 1) |
| 975 | HLA-A*01:01_QSEKEPGQQY | 830 | RHXF2 | ENSG00000131721 | Rhox homeobox family member 2 (Paired-like |
| | | | | | homeobox protein PEPP-2)(Testis homeobox |
| | | | | | gene 1) |
| 976 | HLA-B*44:03_SEKEPGQQY | 831 | RHXF2 | ENSG00000131721 | Rhox homeobox family member 2 (Paired-like |
| | | | | | homeobox protein PEPP-2)(Testis homeobox |
| | | | | | gene 1) |
| 977 | HLA-C*02:02_SEKEPGQQY | 831 | RHXF2 | ENSG00000131721 | Rhox homeobox family member 2 (Paired-like |
| | | | | | homeobox protein PEPP-2)(Testis homeobox |
| | | | | | gene 1) |
| 978 | HLA-B*35:01_LPVLENVSY | 832 | NYAP2 | ENSG00000144460 | Neuronal tyro sine-phosphorylated |
| | | | | | phosphoinositide-3-kinase adapter 2 |
| 979 | HLA-B*35:01_MVNAAVNTY | 833 | NYAP2 | ENSG00000144460 | Neuronal tyrosine-phosphorylated |
| | | | | | phosphoinositide-3-kinase adapter 2 |
| 980 | HLA-A*02:01_FLIEQIDVL | 834 | SUN3 | ENSG00000164744 | SUN domain-containing protein 3 (Sad1/unc-84 |
| | | | | | domain-containing protein 1) |
| 981 | HLA-A*24:02_SYLPGLLYKF | 835 | ZPLD1 | ENSG00000170044 | Zona pellucida-like domain-containing protein 1 |
| | | | | | (ZP domain-containing protein 1) |
| 982 | HLA-A*24:02_LYASWMYQL | 836 | TEX19 | ENSG00000182459 | Testis-expressed protein 19 |
| 983 | HLA-A*11:01_AAGIIVIAK | 837 | KCNH7 | ENSG00000184611 | Potassium voltage-gated channel subfamily H |
| | | | | | member 7 (Ether-a-go-go-related gene potassium |
| | | | | | channel 3)(ERG-3)(Eag-related protein 3) |
| | | | | | (Ether-a-go-go-related protein 3)(hERG-3) |
| | | | | | (Voltage-gated potassium channel subunit |
| | | | | | Kv11.3) |
| 984 | HLA-A*11:01_HVSDPGLPGK | 838 | KCNH7 | ENSG00000184611 | Potassium voltage-gated channel subfamily H |
| | | | | | member 7 (Ether-a-go-go-related gene potassium |
| | | | | | channel 3)(ERG-3)(Eag-related protein 3) |
| | | | | | (Ether-a-go-go-related protein 3)(hERG-3) |
| | | | | | (Voltage-gated potassium channel subunit |
| | | | | | Kv11.3) |
| 985 | HLA-A*29:02_IFGEMVHLY | 839 | KCNH7 | ENSG00000184611 | Potassium voltage-gated channel subfamily H |
| | | | | | member 7 (Ether-a-go-go-related gene potassium |
| | | | | | channel 3)(ERG-3)(Eag-related protein 3) |
| | | | | | (Ether-a-go-go-related protein 3)(hERG-3) |
| | | | | | (Voltage-gated potassium channel subunit |
| | | | | | Kv11.3) |
| 986 | HLA-A*11:01_IVDSSPGIGK | 840 | KCNH7 | ENSG00000184611 | Potassium voltage-gated channel subfamily H |
| | | | | | member 7 (Ether-a-go-go-related gene potassium |
| | | | | | channel 3)(ERG-3)(Eag-related protein 3) |
| | | | | | (Ether-a-go-go-related protein 3)(hERG-3) |
| | | | | | (Voltage-gated potassium channel subunit |
| | | | | | Kv11.3) |
| 987 | HLA-A*01:01_TSDSNLNKY | 841 | KCNH7 | ENSG00000184611 | Potassium voltage-gated channel subfamily H |
| | | | | | member 7 (Ether-a-go-go-related gene potassium |
| | | | | | channel 3)(ERG-3)(Eag-related protein 3) |
| | | | | | (Ether-a-go-go-related protein 3)(hERG-3) |
| | | | | | (Voltage-gated potassium channel subunit |
| | | | | | Kv11.3) |
| 988 | HLA-A*11:01_VSDPGLPGK | 842 | KCNH7 | ENSG00000184611 | Potassium voltage-gated channel subfamily H |
| | | | | | member 7 (Ether-a-go-go-related gene potassium |
| | | | | | channel 3)(ERG-3)(Eag-related protein 3) |
| | | | | | (Ether-a-go-go-related protein 3)(hERG-3) |
| | | | | | (Voltage-gated potassium channel subunit |
| | | | | | Kv11.3) |
| 989 | HLA-B*44:03_AEAELTGGSEW | 843 | C9J420 | ENSG00000186038 | 5-hydroxytryptamine receptor 3E (Fragment) |
| 990 | HLA-B*51:01_LPTSGTPLI | 844 | C9J420 | ENSG00000186038 | 5-hydroxytryptamine receptor 3E (Fragment) |
| 991 | HLA-B*35:01_VPTQVNISF | 845 | C9J420 | ENSG00000186038 | 5-hydroxyhyptamine receptor 3E (Fragment) |
| 992 | HLA-A*03:01_SVSGLVLSH | 613 | S22AO | ENSG00000197658 | Solute carrier family 22 member 24 |
| 993 | HLA-C*02:02_TESSVKDPVAW | 846 | MAGB1 | ENSG00000214107 | Melanoma-associated antigen B1 (Cancer/testis |
| | | | | | antigen 3.1)(CT3.1)(DSS-AHC critical interval |
| | | | | | MAGE superfamily 10)(DAM10)(MAGE-Bl |
| | | | | | antigen)(MAGE-XP antigen) |
| 994 | HLA-A*03:01_RSYSPAPGKQK | 847 | NOBOX | ENSG00000106410 | Homeobox protein NOBOX |
| 995 | HLA-B*35:01_EAAPESLDVVY | 848 | R113B | ENSG00000139797 | RING finger protein 113B (Zinc finger protein |
| | | | | | 183-like 1) |
| 996 | HLA-A*29:02_LALSIGTPYRY | 849 | KCNH5 | ENSG00000140015 | Potassium voltage-gated channel subfamily H |
| | | | | | member 5 (Ether-a-go-go potassium channel 2) |
| | | | | | (hEAG2)(Voltage-gated potassium channel |
| | | | | | subunit Kv10.2) |
| 997 | HLA-B*51:01_MPLQVPPQI | 850 | KCNH5 | ENSG00000140015 | Potassium voltage-gated channel subfamily H |
| | | | | | member 5 (Ether-a-go-go potassium channel 2) |
| | | | | | (hEAG2)(Voltage-gated potassium channel |
| | | | | | subunit Kv10.2) |
| 998 | HLA-A*31:01_QVKIWFQNR | 110 | GBX1 | ENSG00000164900 | Homeobox protein GBX-1 (Gastrulation and |
| | | | | | brain-specific homeobox protein 1) |
| 999 | HLA-B*51:01_MAYEKRVLI | 851 | SG1D1 | ENSG00000168515 | Secretoglobin family 1D member 1 (Lipophilin- |
| | | | | | A) |
| 1000 | HLA-A*24:02_VYTVWTALW | 852 | NKAI3 | ENSG00000185942 | Sodium/potassium-transporting ATPase subunit |
| | | | | | beta-1-interacting protein 3 (NaH/K(+)- |
| | | | | | transporting ATPase subunit beta-1-interacting |
| | | | | | protein 3)(Protein FAM77D) |
| 1001 | HLA-B*44:03_KEADPTGHSY | 650 | MAGA1 | ENSG00000198681 | Melanoma-associated antigen 1 (Antigen MZ2-E) |
| | | | | | (Cancer/testis antigen 1.1)(CT1.1)(MAGE-1 |
| | | | | | antigen) |
| 1002 | HLA-A*11:01_GSFGGVLQK | 853 | SKOR2 | ENSG00000215474 | SKI family transcriptional corepressor 2 |
| | | | | | (Functional Smad-suppressing element on |
| | | | | | chromosome 18)(Fussel-18)(LBX1 corepressor |
| | | | | | 1-like protein)(Ladybird homeobox corepressor |
| | | | | | 1-like protein) |
| 1003 | HLA-B*51:01_IPYAASLI | 854 | SKOR2 | ENSG00000215474 | SKI family transcriptional corepressor 2 |
| | | | | | (Functional Smad-suppressing element on |
| | | | | | chromosome 18)(Fussel-18)(LBX1 corepressor |
| | | | | | 1-like protein)(Ladybird homeobox corepressor |
| | | | | | 1-like protein) |
| 1004 | HLA-A*02:07_FLYEVFAQL | 855 | A0A1B0GTN1 | ENSG00000224960 | Putative SMEK homolog 3 |
| 1005 | HLA-C*16:01_NASTRNVVF | 856 | PTX4 | ENSG00000251692 | Pentraxin-4 |
| 1006 | HLA-A*11:01_QTQLIPVQK | 857 | ZP4 | ENSG00000116996 | Zona pellucida sperm-binding protein 4 (Zona |
| | | | | | pellucida glycoprotein 4)(Zp-4)(Zona pellucida |
| | | | | | protein B)[Cleaved into: Processed zona |
| | | | | | pellucida sperm-binding protein 41 |
| 1007 | HLA-B*44:03_AEDLAKAQRW | 858 | Q5T1N2 | ENSG00000162641 | Protein AKNAD1 |
| 1008 | HLA-A*11:01_KSYQGQSPQK | 859 | Q5T1N2 | ENSG00000162641 | Protein AKNAD1 |
| 1009 | HLA-B*51:01_LPYDGDLSQI | 860 | Q5T1N2 | ENSG00000162641 | Protein AKNAD1 |
| 1010 | HLA-A*11:01_SSSSYIFQK | 861 | Q5T1N2 | ENSG00000162641 | Protein AKNAD1 |
| 1011 | HLA-B*44:03_MEDESNKLW | 862 | ESPB1 | ENSG00000169393 | Epididymal sperm-binding protein 1 (Epididymal |
| | | | | | secretoiy protein 12)(hE12) |
| 1012 | HLA-A*29:02_IVGENSLEY | 863 | ZFP42 | ENSG00000179059 | Zinc finger protein 42 homolog (Zfp-42) |
| | | | | | (Reduced expression protein 1)(REX-1)(hREX- |
| | | | | | 1)(Zinc finger protein 754) |
| 1013 | HLA-B*51:01_LPKLPIPYI | 864 | PSG9 | ENSG00000183668 | Pregnancy-specific beta-1-glycoprotein 9 (PS- |
| | | | | | beta-G-9)(PSBG-9)(Pregnancy-specific |
| | | | | | glycoprotein 9)(PS34)(Pregnancy-specific beta- |
| | | | | | 1 glycoprotein B)(PS-beta-B)(Pregnancy- |
| | | | | | specific beta-1-glycoprotein 11)(PS-beta-G-11) |
| | | | | | (PSBG-11)(Pregnancy-specific glycoprotein 11) |
| | | | | | (Pregnancy-specific glycoprotein 7)(PSG7) |
| 1014 | HLA-B*44:03_QEWTDYRLTW | 865 | ACHB4 | ENSG00000117971 | Neuronal acetylcholine receptor subunit beta-4 |
| 1015 | HLA-B*46:01_AAAAAAATY | 372 | NKX24 | ENSG00000125816 | Homeobox protein Nkx-2.4 (Homeobox protein |
| | | | | | NK-2 homolog D) |
| 1016 | HLA-B*51:01_DANLANTII | 866 | SPI2A | ENSG00000147059 | Spindlin-2A (Protein DXF34)(Spindlin-like |
| | | | | | protein 2A)(SPIN-2)(SPIN-2A) |
| 1017 | HLA-B*51:01_DANLANTI | 867 | SPI2A | ENSG00000147059 | Spindlin-2A (Protein DXF34)(Spindlin-like |
| | | | | | protein 2A)(SPIN-2)(SPIN-2A) |
| 1018 | HLA-B*44:03_KEGDEPITQW | 868 | SPI2A | ENSG00000147059 | Spindlin-2A (Protein DXF34)(Spindlin-like |
| | | | | | protein 2A)(SPIN-2)(SPIN-2A) |
| 1019 | HLA-B*51:01_DPMLTAAAI | 869 | SL9C1 | ENSG00000172139 | Sodium/hydrogen exchanger 10 (Na(+)/H(+) |
| | | | | | exchanger 10)(NHE-10)(Solute carrier family 9 |
| | | | | | member 10)(Solute carrier family 9 member Cl) |
| | | | | | (Sperm-specific Na(+)/H(+) exchanger)(sNHE) |
| 1020 | HLA-B*18:01_EEFEHVGY | 870 | SL9C1 | ENSG00000172139 | Sodium/hydrogen exchanger 10 (Na(+)/H(+) |
| | | | | | exchanger 10)(NHE-10)(Solute carrier family 9 |
| | | | | | member 10)(Solute carrier family 9 member Cl) |
| | | | | | (Sperm-specific Na(+)/H(+) exchanger)(sNHE) |
| 1021 | HLA-B*51:01_FPIPVPVI | 871 | SL9C1 | ENSG00000172139 | Sodium/hydrogen exchanger 10 (Na(+)/H(+) |
| | | | | | exchanger 10)(NHE-10)(Solute carrier family 9 |
| | | | | | member 10)(Solute carrier family 9 member Cl) |
| | | | | | (Sperm-specific Na(+)/H(+) exchanger)(sNHE) |
| 1022 | HLA-B*18:01_NETEVIVF | 872 | SL9C1 | ENSG00000172139 | Sodium/hydrogen exchanger 10 (Na(+)/H(+) |
| | | | | | exchanger 10)(NHE-10)(Solute carrier family 9 |
| | | | | | member 10)(Solute carrier family 9 member Cl) |
| | | | | | (Sperm-specific Na(+)/H(+) exchanger)(sNHE) |
| 1023 | HLA-B*44:03_SESQKTVTF | 873 | SL9C1 | ENSG00000172139 | Sodium/hydrogen exchanger 10 (Na(+)/H(+) |
| | | | | | exchanger 10)(NHE-10)(Solute carrier family 9 |
| | | | | | member 10)(Solute carrier family 9 member Cl) |
| | | | | | (Sperm-specific Na(+)/H(+) exchanger)(sNHE) |
| 1024 | HLA-C*02:02_SESQKTVTF | 873 | SL9C1 | ENSG00000172139 | Sodium/hydrogen exchanger 10 (Na(+)/H(+) |
| | | | | | exchanger 10)(NHE-10)(Solute carrier family 9 |
| | | | | | member 10)(Solute carrier family 9 member Cl) |
| | | | | | (Sperm-specific Na(+)/H(+) exchanger)(sNHE) |
| 1025 | HLA-B*35:01_EPLSVTAKY | 874 | VCX1 | ENSG00000182583 | Variable charge X-linked protein 1 (Variable |
| | | | | | charge protein on X with ten repeats)(VCX-10r) |
| | | | | | (Variably charged protein X-B1)(VCX-B1) |
| 1026 | HLA-C*02:02_TEVVEGKEW | 272 | NPSR1 | ENSG00000187258 | Neuropeptide S receptor (G-protein coupled |
| | | | | | receptor 154)(G-protein coupled receptor |
| | | | | | PGR14)(G-protein coupled receptor for asthma |
| | | | | | susceptibility) |
| 1027 | HLA-A*24:02_VYLPKIPSW | 875 | E5RK72 | ENSG00000215262 | Potassium channel subfamily U member 1 |
| 1028 | HLA-B*51:01_MPLLPSTV | 876 | CRSPL | ENSG00000101074 | Peptidase inhibitor R3HDML (Cysteine-rich |
| | | | | | secretory protein R3HDML) |
| 1029 | HLA-A*29:02_NFPQFPETLSY | 877 | MROH9 | ENSG00000117501 | Maestro heat-like repeat-containing protein |
| | | | | | family member 9 |
| 1030 | HLA-B*18:01_NESSLVEQM | 878 | NDST4 | ENSG00000138653 | Bifunctional heparan sulfate N-deacetylase/N- |
| | | | | | sulfotransferase 4 (EC 2.8.2.8)(Glucosaminyl N- |
| | | | | | deacetylase/N-sulfotransferase 4)(NDST-4)(N- |
| | | | | | heparan sulfate sulfotransferase 4)(N-HSST 4) |
| | | | | | [Includes: Heparan sulfate N-deacetylase 4 (EC |
| | | | | | 3.-.-.-); Heparan sulfate N-sulfotransferase 4 (EC |
| | | | | | 2.8.2.-)] |
| 1031 | HLA-A*11:01_KTITHIVAK | 879 | SHP1L | ENSG00000157060 | Testicular spindle-associated protein SHCBP1L |
| | | | | | (SHC SH2 domain-binding protein 1-like protein) |
| 1032 | HLA-A*02:07_LVDEILEEL | 880 | SHP1L | ENSG00000157060 | Testicular spindle-associated protein SHCBP1L |
| | | | | | (SHC SH2 domain-binding protein 1-like protein) |
| 1033 | HLA-A*11:01_GTQDPGLLVPK | 881 | SP7 | ENSG00000170374 | Transcription factor Sp7 (Zinc finger protein |
| | | | | | osterix) |
| 1034 | HLA-B*44:03_EEIVLGQRL | 882 | XKR3 | ENSG00000172967 | XK-related protein 3 (X Kell blood group-related |
| | | | | | 3)(XTES) |
| 1035 | HLA-A*26:01_EVISRVVTL | 883 | XKR3 | ENSG00000172967 | XK-related protein 3 (X Kell blood group-related |
| | | | | | 3)(XTES) |
| 1036 | HLA-A*11:01_NTYASTLYK | 884 | FGF16 | ENSG00000196468 | Fibroblast growth factor 16 (FGF-16) |
| 1037 | HLA-A*26:01_EVISVVLKY | 885 | TRPC7 | ENSG00000069018 | Short transient receptor potential channel 7 |
| | | | | | (TipC7)(Transient receptor protein 7)(TRP-7) |
| | | | | | (hTRP7) |
| 1038 | HLA-A*29:02_EVISVVLKY | 885 | TRPC7 | ENSG00000069018 | Short transient receptor potential channel 7 |
| | | | | | (TipC7)(Transient receptor protein 7)(TRP-7) |
| | | | | | (hTRP7) |
| 1039 | HLA-B*18:01_IETEFKNDY | 886 | TRPC7 | ENSG00000069018 | Short transient receptor potential channel 7 |
| | | | | | (TrpC7)(Transient receptor protein 7)(TRP-7) |
| | | | | | (hTRP7) |
| 1040 | HLA-B*44:02_SEKEPGQQY | 831 | RHXF2 | ENSG00000131721 | Rhox homeobox family member 2 (Paired-like |
| | | | | | homeobox protein PEPP-2)(Testis homeobox |
| | | | | | gene 1) |
| 1041 | HLA-A*29:02_IVISAYFLY | 887 | NDST4 | ENSG00000138653 | Bifunctional heparan sulfate N-deacetylase/N- |
| | | | | | sulfotransferase 4 (EC 2.8.2.8)(Glucosaminyl N- |
| | | | | | deacetylase/N-sulfotransferase 4)(NDST-4)(N- |
| | | | | | heparan sulfate sulfotransferase 4)(N-HSST 4) |
| | | | | | [Includes: Heparan sulfate N-deacetylase 4 (EC |
| | | | | | 3.-.-.-); Heparan sulfate N-sulfotransferase 4 (EC |
| | | | | | 2.8.2.-)] |
| 1042 | HLA-A*02:07_VMDEVQKFL | 888 | NDST4 | ENSG00000138653 | Bifunctional heparan sulfate N-deacetylase/N- |
| | | | | | sulfotransferase 4 (EC 2.8.2.8)(Glucosaminyl N- |
| | | | | | deacetylase/N-sulfotransferase 4)(NDST-4)(N- |
| | | | | | heparan sulfate sulfotransferase 4)(N-HSST 4) |
| | | | | | [Includes: Heparan sulfate N-deacetylase 4 (EC |
| | | | | | 3.-.-.-); Heparan sulfate N-sulfotransferase 4 (EC |
| | | | | | 2.8.2.-)] |
| 1043 | HLA-A*29:02_AFPHHPMGMLY | 889 | X6R6V8 | ENSG00000143552 | Nuclear pore membrane glycoprotein 210-like |
| 1044 | HLA-A*11:01_ASMSVTPVYK | 890 | X6R6V8 | ENSG00000143552 | Nuclear pore membrane glycoprotein 210-like |
| 1045 | HLA-A*30:02_ASMSVTPVY | 891 | X6R6V8 | ENSG00000143552 | Nuclear pore membrane glycoprotein 210-like |
| 1046 | HLA-B*18:01_DEVQILVF | 892 | X6R6V8 | ENSG00000143552 | Nuclear pore membrane glycoprotein 210-like |
| 1047 | HLA-A*33:01_DVINSIEIVSR | 893 | X6R6V8 | ENSG00000143552 | Nuclear pore membrane glycoprotein 210-like |
| 1048 | HLA-A*26:01_ETFSLVEGSGY | 894 | X6R6V8 | ENSG00000143552 | Nuclear pore membrane glycoprotein 210-like |
| 1049 | HLA-A*33:01_EYSENYILR | 895 | X6R6V8 | ENSG00000143552 | Nuclear pore membrane glycoprotein 210-like |
| 1050 | HLA-A*24:02_FYNSIGEKF | 896 | X6R6V8 | ENSG00000143552 | Nuclear pore membrane glycoprotein 210-like |
| 1051 | HLA-B*35:01_HPASMSVTPVY | 897 | X6R6V8 | ENSG00000143552 | Nuclear pore membrane glycoprotein 210-like |
| 1052 | HLA-B*18:01_IEIPIAMY | 898 | X6R6V8 | ENSG00000143552 | Nuclear pore membrane glycoprotein 210-like |
| 1053 | HLA-A*02:01_ILDDKTAMV | 899 | X6R6V8 | ENSG00000143552 | Nuclear pore membrane glycoprotein 210-like |
| 1054 | HLA-A*02:07_ILDDKTAMV | 899 | X6R6V8 | ENSG00000143552 | Nuclear pore membrane glycoprotein 210-like |
| 1055 | HLA-A*01:01_ISDNLRITY | 900 | X6R6V8 | ENSG00000143552 | Nuclear pore membrane glycoprotein 210-like |
| 1056 | HLA-A*11:01_IVTGVGVAR | 901 | X6R6V8 | ENSG00000143552 | Nuclear pore membrane glycoprotein 210-like |
| 1057 | HLA-A*24:02_IYNHPDVKETF | 902 | X6R6V8 | ENSG00000143552 | Nuclear pore membrane glycoprotein 210-like |
| 1058 | HLA-A*24:02_KYLESSATF | 903 | X6R6V8 | ENSG00000143552 | Nuclear pore membrane glycoprotein 210-like |
| 1059 | HLA-B*51:01_LPFHADVEI | 904 | X6R6V8 | ENSG00000143552 | Nuclear pore membrane glycoprotein 210-like |
| 1060 | HLA-A*02:01_LQTDIVTGV | 905 | X6R6V8 | ENSG00000143552 | Nuclear pore membrane glycoprotein 210-like |
| 1061 | HLA-B*35:01_NASLTSIIY | 906 | X6R6V8 | ENSG00000143552 | Nuclear pore membrane glycoprotein 210-like |
| 1062 | HLA-A*02:01_QLASAIVTL | 907 | X6R6V8 | ENSG00000143552 | Nuclear pore membrane glycoprotein 210-like |
| 1063 | HLA-A*11:01_QTNLVFVHK | 908 | X6R6V8 | ENSG00000143552 | Nuclear pore membrane glycoprotein 210-like |
| 1064 | HLA-A*11:01_QTTLVAIAK | 909 | X6R6V8 | ENSG00000143552 | Nuclear pore membrane glycoprotein 210-like |
| 1065 | HLA-B*44:03_SEAVVVRAM | 910 | X6R6V8 | ENSG00000143552 | Nuclear pore membrane glycoprotein 210-like |
| 1066 | HLA-B*18:01_SEQGVVTITY | 911 | X6R6V8 | ENSG00000143552 | Nuclear pore membrane glycoprotein 210-like |
| 1067 | HLA-B*44:02_SEQGVVTITY | 911 | X6R6V8 | ENSG00000143552 | Nuclear pore membrane glycoprotein 210-like |
| 1068 | HLA-B*44:03_SEQGVVTITY | 911 | X6R6V8 | ENSG00000143552 | Nuclear pore membrane glycoprotein 210-like |
| 1069 | HLA-C*02:02_SEQGVVTITY | 911 | X6R6V8 | ENSG00000143552 | Nuclear pore membrane glycoprotein 210-like |
| 1070 | HLA-A*02:01_SLGHTLVTV | 912 | X6R6V8 | ENSG00000143552 | Nuclear pore membrane glycoprotein 210-like |
| 1071 | HLA-A*26:01_STASIFLAY | 913 | X6R6V8 | ENSG00000143552 | Nuclear pore membrane glycoprotein 210-like |
| 1072 | HLA-A*29:02_STASIFLAY | 913 | X6R6V8 | ENSG00000143552 | Nuclear pore membrane glycoprotein 210-like |
| 1073 | HLA-B*35:01_TPMEQQDEY | 914 | X6R6V8 | ENSG00000143552 | Nuclear pore membrane glycoprotein 210-like |
| 1074 | HLA-A*29:02_VFEKLQLFY | 915 | X6R6V8 | ENSG00000143552 | Nuclear pore membrane glycoprotein 210-like |
| 1075 | HLA-A*24:02_VYVITVDVF | 916 | X6R6V8 | ENSG00000143552 | Nuclear pore membrane glycoprotein 210-like |
| 1076 | HLA-A*02:07_YVDDSPLEL | 917 | X6R6V8 | ENSG00000143552 | Nuclear pore membrane glycoprotein 210-like |
| 1077 | HLA-C*05:01_YVDDSPLEL | 917 | X6R6V8 | ENSG00000143552 | Nuclear pore membrane glycoprotein 210-like |
| 1078 | HLA-A*01:01_ETDALDIDY | 918 | CNTP5 | ENSG00000155052 | Contactin-associated protein-like 5 (Cell |
| | | | | | recognition molecule Caspr5) |
| 1079 | HLA-A*11:01_GTQSTHESLK | 919 | I22R2 | ENSG00000164485 | Interleukin-22 receptor subunit alpha-2 (IL-22 |
| | | | | | receptor subunit alpha-2)(IL-22R-alpha-2)(IL- |
| | | | | | 22RA2)(Cytokine receptor class-II member 10) |
| | | | | | (Cytokine receptor family 2 member 10)(CRF2- |
| | | | | | 10)(Cytokine receptor family type 2, soluble 1) |
| | | | | | (CRF2-S1)(Interleukin-22-binding protein)(IL- |
| | | | | | 22BP)(IL22BP)(ZcytoR16) |
| 1080 | HLA-A*02:01_QLLDGFMITL | 920 | PASD1 | ENSG00000166049 | Circadian clock protein PASD1 (Cancer/testis |
| | | | | | antigen 63)(CT63)(OX-TES-1)(PAS domain- |
| | | | | | containing protein 1) |
| 1081 | HLA-A*01:01_KTELETALY | 921 | GG6L2 | ENSG00000174450 | Golgin subfamily A member 6-like protein 2 |
| 1082 | HLA-B*51:01_LPPSLQSSL | 922 | GG6L2 | ENSG00000174450 | Golgin subfamily A member 6-like protein 2 |
| 1083 | HLA-C*02:02_AESPLEVPQSF | 626 | MAGBG | ENSG00000189023 | Melanoma-associated antigen B16 (MAGE-B16 |
| | | | | | antigen) |
| 1084 | HLA-B*46:01_AGMTIATSY | 250 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD 6p24.1 provirus ancestral Env |
| | | | | | polyprotein)[Cleaved into: Surface protein (SU); |
| | | | | | Transmembrane protein (TM)] |
| 1085 | HLA-A*24:02_VYGDPHYVTF | 923 | ZAN | ENSG00000146839 | Zonadhesin |
| 1086 | HLA-A*29:02_LFWKPLRY | 924 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 1087 | HLA-B*08:01_QVLVKKISQ | 925 | S100G | ENSG00000169906 | Protein S100-G (Calbindin-D9k)(S100 calcium- |
| | | | | | binding protein G)(Vitamin D-dependent |
| | | | | | calcium-binding protein, intestinal)(CABP) |
| 1088 | HLA-B*27:02_GRTVAVAEY | 926 | KR204 | ENSG00000206105 | Putative keratin-associated protein 20-4 |
| 1089 | HLA-B*51:01_DAPSKGPSI | 927 | CL071 | ENSG00000214700 | Uncharacterized protein C12orf71 |
| 1090 | HLA-A*02:07_YVDNVSARV | 928 | GFRA4 | ENSG00000125861 | GDNF family receptor alpha-4 (GDNF receptor |
| | | | | | alpha-4)(GDNER-alpha-4)(GER-alpha-4) |
| | | | | | (Persephin receptor) |
| 1091 | HLA-A*01:01_VLDDGSIDY | 929 | LYZL2 | ENSG00000151033 | Lysozyme-like protein 2 (Lysozyme-2)(EC |
| | | | | | 3.2.1.17) |
| 1092 | HLA-A*30:02_AVFEAGTSVTY | 930 | CNTP5 | ENSG00000155052 | Contactin-associated protein-like 5 (Cell |
| | | | | | recognition molecule Caspr5) |
| 1093 | HLA-A*02:01_FLIPYVIAL | 931 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 1094 | HLA-A*29:02_FVSPKGVLAY | 932 | S7A13 | ENSG00000164893 | Solute carrier family 7 member 13 (Sodium- |
| | | | | | independent aspartate/glutamate transporter 1) |
| | | | | | (X-amino acid transporter 2) |
| 1095 | HLA-A*30:02_GQALLLAEY | 933 | S7A13 | ENSG00000164893 | Solute carrier family 7 member 13 (Sodium- |
| | | | | | independent aspartate/glutamate transporter 1) |
| | | | | | (X-amino acid transporter 2) |
| 1096 | HLA-B*35:01_DPYATITY | 934 | SAGE1 | ENSG00000181433 | Sarcoma antigen 1 (Cancer/testis antigen 14) |
| | | | | | (CT14) |
| 1097 | HLA-A*29:02_FFSDFGLLWY | 935 | NALP9 | ENSG00000185792 | NACHT, LRR and PYD domains-containing |
| | | | | | protein 9 (Nucleotide-binding oligomerization |
| | | | | | domain protein 6)(PYRIN and NACHT- |
| | | | | | containing protein 12) |
| 1098 | HLA-B*51:01_FPLATQLNV | 936 | PRR27 | ENSG00000187533 | Proline-rich protein 27 |
| 1099 | HLA-A*01:01_YTDTGLPSY | 937 | PRR27 | ENSG00000187533 | Proline-rich protein 27 |
| 1100 | HLA-A*24:02_IYPTVDREHF | 938 | RFA4 | ENSG00000204086 | Replication protein A 30 kDa subunit (RP-A p30) |
| | | | | | (Replication factor A protein 4)(RF-A protein 4) |
| 1101 | HLA-A*29:02_LLNPVIATY | 939 | CB061 | ENSG00000239605 | Uncharacterized protein C2orf61 |
| 1102 | HLA-A*30:02_LLNPVIATY | 939 | CB061 | ENSG00000239605 | Uncharacterized protein C2orf61 |
| 1103 | HLA-B*46:01_LLNPVIATY | 939 | CB061 | ENSG00000239605 | Uncharacterized protein C2orf61 |
| 1104 | HLA-A*01:01_LTDTPIPGTY | 940 | CB061 | ENSG00000239605 | Uncharacterized protein C2orf61 |
| 1105 | HLA-A*26:01_EVIGPDGIITV | 941 | ROP1A | ENSG00000065371 | Ropporin-1A (Cancer/testis antigen 91)(CT9 I) |
| | | | | | (Rhophilin-associated protein 1A) |
| 1106 | HLA-B*18:01_NESPQTNEF | 942 | TPTE2 | ENSG00000132958 | Phosphatidylinositol 3,4,5-trisphosphate 3- |
| | | | | | phosphatase TPTE2 (EC 3.1.3.67)(Lipid |
| | | | | | phosphatase TPIP)(TPTE and PTEN |
| | | | | | homologous inositol lipid phosphatase) |
| 1107 | HLA-A*29:02_YFAQVKHLY | 943 | TPTE2 | ENSG00000132958 | Phosphatidylinositol 3,4,5-trisphosphate 3- |
| | | | | | phosphatase TPTE2 (EC 3.1.3.67)(Lipid |
| | | | | | phosphatase TPIP)(TPTE and PTEN |
| | | | | | homologous inositol lipid phosphatase) |
| 1108 | HLA-A*33:01_EANSMNTLR | 944 | LGSN | ENSG00000146166 | Lengsin (Glutamate-ammonia ligase domain- |
| | | | | | containing protein 1)(Lens glutamine synthase- |
| | | | | | like) |
| 1109 | HLA-A*01:01_GSDDHQYIY | 945 | DMP1 | ENSG00000152592 | Dentin matrix acidic phosphoprotein 1 (DMP-1) |
| | | | | | (Dentin matrix protein 1) |
| 1110 | HLA-C*02:02_AEVNVKGLF | 946 | LMTD1 | ENSG00000152936 | Lamin tail domain-containing protein 1 |
| | | | | | (Intermediate filament tail domain-containing |
| | | | | | protein 1) |
| 1111 | HLA-A*26:01_ETSDIQEPYY | 947 | I22R2 | ENSG00000164485 | Interleukin-22 receptor subunit alpha-2 (IL-22 |
| | | | | | receptor subunit alpha-2)(IL-22R-alpha-2)(IL- |
| | | | | | 22RA2)(Cytokine receptor class-II member 10) |
| | | | | | (Cytokine receptor family 2 member 10)(CRF2- |
| | | | | | 10)(Cytokine receptor family type 2, soluble 1) |
| | | | | | (CRF2-S1)(Interleukin-22-binding protein)(IL- |
| | | | | | 22BP)(IL22BP)(ZcytoR16) |
| 1112 | HLA-A*02:07_LVIDTVTEV | 302 | SPERT | ENSG00000174015 | Spermatid-associated protein (Protein chibby |
| | | | | | homolog 2) |
| 1113 | HLA-A*11:01_ASQKAIIFK | 948 | MAGB6 | ENSG00000176746 | Melanoma-associated antigen B6 (Cancer/testis |
| | | | | | antigen 3.4)(CT3.4)(MAGE-B6 antigen) |
| 1114 | HLA-A*01:01_ITEDLVQDKY | 949 | MAGB6 | ENSG00000176746 | Melanoma-associated antigen B6 (Cancer/testis |
| | | | | | antigen 3.4)(CT3.4)(MAGE-B6 antigen) |
| 1115 | HLA-C*02:02_KEMDSSGESY | 950 | MAGB6 | ENSG00000176746 | Melanoma-associated antigen B6 (Cancer/testis |
| | | | | | antigen 3.4)(CT3.4)(MAGE-B6 antigen) |
| 1116 | HLA-A*26:01_ELYEGTLGKY | 951 | SG1C1 | ENSG00000188076 | Secretoglobin family 1C member 1 |
| | | | | | (Secretoglobin RYD5) |
| 1117 | HLA-B*08:01_QPMHKAEL | 952 | SG1C1 | ENSG00000188076 | Secretoglobin family 1C member 1 |
| | | | | | (Secretoglobin RYD5) |
| 1118 | HLA-B*44:02_EEIENLYRF | 222 | MEIG1 | ENSG00000197889 | Meiosis expressed gene 1 protein homolog |
| 1119 | HLA-B*51:01_IPILQKPLI | 953 | NANGN | ENSG00000205857 | NANOG neighbor homeobox (Homeobox protein |
| | | | | | C14) |
| 1120 | HLA-B*44:02_SEDEQNGKQKW | 954 | NANGN | ENSG00000205857 | NANOG neighbor homeobox (Homeobox protein |
| | | | | | C14) |
| 1121 | HLA-B*44:03_SEDEQNGKQKW | 954 | NANGN | ENSG00000205857 | NANOG neighbor homeobox (Homeobox protein |
| | | | | | C14) |
| 1122 | HLA-C*02:02_SEDEQNGKQKW | 954 | NANGN | ENSG00000205857 | NANOG neighbor homeobox (Homeobox protein |
| | | | | | C14) |
| 1123 | HLA-A*02:01_WLTPVIPAL | 955 | NANGN | ENSG00000205857 | NANOG neighbor homeobox (Homeobox protein |
| | | | | | C14) |
| 1124 | HLA-B*27:02_TLQSILAIVK | 956 | GNAT3 | ENSG00000214415 | Guanine nucleotide-binding protein G(t) subunit |
| | | | | | alpha-3 (Gustducin alpha-3 chain) |
| 1125 | HLA-C*05:01_IADIVTSVF | 957 | A0A1B0GTN1 | ENSG00000224960 | Putative SMEK homolog 3 |
| 1126 | HLA-B*44:03_NEAYIPKLL | 958 | A0A1B0GTN1 | ENSG00000224960 | Putative SMEK homolog 3 |
| 1127 | HLA-C*04:01_TYDEQFQGM | 959 | A0A1B0GTN1 | ENSG00000224960 | Putative SMEK homolog 3 |
| 1128 | HLA-B*18:01_DEDSIFAHY | 960 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 1129 | HLA-A*11:01_ITQGNTLLR | 961 | CC049 | ENSG00000163632 | Putative uncharacterized protein C3orf49 |
| 1130 | HLA-B*44:03_WEVDVEKATRW | 962 | TRIMM | ENSG00000179046 | Probable E3 ubiquitin-protein ligase TRIML2 |
| | | | | | (EC 2.3.2.27)(RING-type E3 ubiquitin |
| | | | | | transferase TRIML2)(SPRY domain-containing |
| | | | | | protein 6)(Tripartite motif family-like protein 2) |
| 1131 | HLA-A*02:07_ALPNKLEEL | 963 | UTS2B | ENSG00000188958 | Urotensin-2B (Urotensin II-related peptide) |
| | | | | | (Urotensin IIB)(U-IIB)(UIIB)(Urotensin-2 |
| | | | | | domain-containing protein) |
| 1132 | HLA-B*44:03_NEIFPDKKY | 964 | UTS2B | ENSG00000188958 | Urotensin-2B (Urotensin II-related peptide) |
| | | | | | (Urotensin IIB)(U-IIB)(UIIB)(Urotensin-2 |
| | | | | | domain-containing protein) |
| 1133 | HLA-B*51:01_NPYFQNKVI | 965 | TSPY1 | ENSG00000258992 | Testis-specific Y-encoded protein 1 (Cancer/testis |
| | | | | | antigen 78)(CT78) |
| 1134 | HLA-A*30:02_TTAPGTVHSY | 403 | Q5JUY5 | ENSG00000117400 | Thrombopoietin receptor |
| 1135 | HLA-B*51:01_LPALLASLI | 966 | PERL | ENSG00000167419 | Lactoperoxidase (LPO)(EC 1.11.1.7)(Salivary |
| | | | | | peroxidase)(SPO) |
| 1136 | HLA-B*44:02_SEFEQIRLF | 491 | TRI60 | ENSG00000176979 | Tripartite motif-containing protein 60 (RING |
| | | | | | finger protein 129)(RING finger protein 33) |
| 1137 | HLA-A*02:01_LLSQDILQV | 967 | ADIG | ENSG00000182035 | Adipogenin |
| 1138 | HLA-A*30:02_GQHGSGSSYSY | 968 | HORN | ENSG00000197915 | Hornerin |
| 1139 | HLA-B*44:02_AEDFKIQNW | 969 | CP096 | ENSG00000205832 | Uncharacterized protein C16orf96 |
| 1140 | HLA-A*26:01_EIYEILSPSY | 970 | CP096 | ENSG00000205832 | Uncharacterized protein C16orf96 |
| 1141 | HLA-A*26:01_EVASLQNKF | 971 | CP096 | ENSG00000205832 | Uncharacterized protein C16orf96 |
| 1142 | HLA-A*11:01_GTTVDILQK | 972 | CP096 | ENSG00000205832 | Uncharacterized protein C16orf96 |
| 1143 | HLA-B*35:01_IAAAAAAAY | 973 | CP096 | ENSG00000205832 | Uncharacterized protein C16orf96 |
| 1144 | HLA-A*02:07_LLPPLIPSL | 974 | CP096 | ENSG00000205832 | Uncharacterized protein C16orf96 |
| 1145 | HLA-B*35:01_YPYGDPHVIDY | 975 | CP096 | ENSG00000205832 | Uncharacterized protein C16orf196 |
| 1146 | HLA-B*51:01_YPYGDPHVI | 976 | CP096 | ENSG00000205832 | Uncharacterized protein C16orf96 |
| 1147 | HLA-A*29:02_YFAQVKHLY | 943 | TPTE | ENSG00000274391 | Putative tyrosine-protein phosphatase TPTE (EC |
| | | | | | 3.1.3.48)(Cancer/testis antigen 44)(CT44) |
| | | | | | (Transmembrane phosphatase with tensin |
| | | | | | homology)(Tumor antigen BJ-HCC-5) |
| 1148 | HLA-A*11:01_RSYSPAPGK | 977 | NOBOX | ENSG00000106410 | Homeobox protein NOBOX |
| 1149 | HLA-A*30:02_AQKNTFVSY | 978 | CCD62 | ENSG00000130783 | Coiled-coil domain-containing protein 62 |
| | | | | | (Protein TSP-NY)(Protein aaa) |
| 1150 | HLA-A*02:01_TLSNTLVEL | 979 | CCD62 | ENSG00000130783 | Coiled-coil domain-containing protein 62 |
| | | | | | (Protein TSP-NY)(Protein aaa) |
| 1151 | HLA-A*03:01_RVWNMTATRPK | 980 | FATE1 | ENSG00000147378 | Fetal and adult testis-expressed transcript protein |
| | | | | | (Cancer/testis antigen 43)(CT43)(Tumor antigen |
| | | | | | BJ-HCC-2) |
| 1152 | HLA-B*35:01_LPNTVTDAL | 981 | TERT | ENSG00000164362 | Telomerase reverse transcriptase (EC 2.7.7.49) |
| | | | | | (HEST2)(Telomerase catalytic subunit) |
| | | | | | (Telomerase-associated protein 2)(TP2) |
| 1153 | HLA-A*29:02_YLLGWTTFLLY | 982 | ESPB1 | ENSG00000169393 | Epididymal sperm-binding protein 1 (Epididymal |
| | | | | | secretory protein 12)(hE12) |
| 1154 | HLA-A*03:01_RIFEKYAAK | 983 | S100G | ENSG00000169906 | Protein S100-G (Calbindin-D9k)(S100 calcium- |
| | | | | | binding protein G)(Vitamin D-dependent |
| | | | | | calcium-binding protein, intestinal)(CABP) |
| 1155 | HLA-B*35:01_FPTEVTYTL | 984 | HSF5 | ENSG00000176160 | Heat shock factor protein 5 (HSF 5)(Heat shock |
| | | | | | transcription factor 5)(HSTF 5) |
| 1156 | HLA-B*35:01_NPSPSSVVF | 985 | HSF5 | ENSG00000176160 | Heat shock factor proteins (HSF 5)(Heat shock |
| | | | | | transcription factor 5)(HSTF 5) |
| 1157 | HLA-A*02:07_KVLEFVAKV | 986 | MAGBG | ENSG00000189023 | Melanoma-associated antigen B16 (MAGE-B16 |
| | | | | | antigen) |
| 1158 | HLA-A*11:01_SSSEQSPLQK | 987 | FOXR2 | ENSG00000189299 | Forkhead box protein R2 (Forkhead box protein |
| | | | | | N6) |
| 1159 | HLA-B*44:02_EEFSLQKSY | 496 | E5RK72 | ENSG00000215262 | Potassium channel subfamily U member 1 |
| 1160 | HLA-A*03:01_SVIGGPSTYK | 419 | ERVV2 | ENSG00000268964 | Endogenous retrovims group V member 2 Env |
| | | | | | polyprotein (HERV-V_19q13.41 provirus |
| | | | | | ancestral Env polyprotein 2) |
| 1161 | HLA-A*26:01_SVIGGPSTY | 988 | ERVV2 | ENSG00000268964 | Endogenous retrovirus group V member 2 Env |
| | | | | | polyprotein (HERV-V_19q13.41 provirus |
| | | | | | ancestral Env polyprotein 2) |
| 1162 | HLA-A*26:01_EIKGTVTEF | 989 | TDRD1 | ENSG00000095627 | Tudor domain-containing protein 1 (Cancer/testis |
| | | | | | antigen 41.1)(CT41.1) |
| 1163 | HLA-A*02:07_LLDHVLIEM | 990 | TDRD1 | ENSG00000095627 | Tudor domain-containing protein 1 (Cancer/testis |
| | | | | | antigen 41.1)(CT41.1) |
| 1164 | HLA-B*44:03_AEQQPQPQF | 653 | MORC1 | ENSG00000114487 | MORC family CW-type zinc finger protein 1 |
| | | | | | (Cancer/testis antigen 33)(CT33) |
| 1165 | HLA-A*30:02_STLPFQSAKY | 991 | OPRD | ENSG00000116329 | Delta-type opioid receptor (D-OR-1)(DOR-1) |
| 1166 | HLA-A*26:01_EVNPTTHSY | 992 | MAGB4 | ENSG00000120289 | Melanoma-associated antigen B4 (MAGE-B4 |
| | | | | | antigen) |
| 1167 | HLA-A*30:02_EVNPTTHSY | 992 | MAGB4 | ENSG00000120289 | Melanoma-associated antigen B4 (MAGE-B4 |
| | | | | | antigen) |
| 1168 | HLA-A*30:02_AAAAAAAAATY | 993 | NKX24 | ENSG00000125816 | Homeobox protein Nkx-2.4 (Homeobox protein |
| | | | | | NK-2 homolog D) |
| 1169 | HLA-A*02:01_FLITGIVQV | 994 | GP119 | ENSG00000147262 | Glucose-dependent insulinotropic receptor (G- |
| | | | | | protein coupled receptor 119) |
| 1170 | HLA-A*02:07_ALDVEFYTL | 995 | SL9C2 | ENSG00000162753 | Sodium/hydrogen exchanger 11 (Na(+)/H(+) |
| | | | | | exchanger 11)(NHE-11)(Solute carrier family 9 |
| | | | | | member 11)(Solute carrier family 9 member C2) |
| 1171 | HLA-C*04:01_IYDVSTYM | 996 | SL9C2 | ENSG00000162753 | Sodium/hydrogen exchanger 11 (Na(-9/H(+) |
| | | | | | exchanger 11)(NHE-11)(Solute carrier family 9 |
| | | | | | member 11)(Solute carrier family 9 member C2) |
| 1172 | HLA-B*51:01_LAYHVQNEI | 997 | TERB2 | ENSG00000167014 | Telomere repeats-binding bouquet formation |
| | | | | | protein 2 |
| 1173 | HLA-A*26:01_FTSSQVQRY | 998 | SL9C1 | ENSG00000172139 | Sodium/hydrogen exchanger 10 (Na(+)/H(+) |
| | | | | | exchanger 10)(NHE-10)(Solute carrier family 9 |
| | | | | | member 10)(Solute carrier family 9 member CO |
| | | | | | (Sperm-specific Na(+)/H(+) exchanger)(sNHE) |
| 1174 | HLA-A*26:01_EVFPQSHHF | 999 | ATS20 | ENSG00000173157 | A disintegrin and metalloproteinase with |
| | | | | | thrombospondin motifs 20 (ADAM-TS 20) |
| | | | | | (ADAM-TS20)(ADAMTS-20)(EC 3.4.24.-) |
| 1175 | HLA-B*35:01_SAWETITIY | 1000 | MC5R | ENSG00000176136 | Melanocortin receptor 5 (MCS-R)(MC-2) |
| 1176 | HLA-A*26:01_EIYGGHHSAF | 1001 | OLIG3 | ENSG00000177468 | Oligodendrocyte transcription factor 3 (01ig03) |
| | | | | | (Class B basic helix-loop-helix protein 7) |
| | | | | | (bHLHb7)(Class E basic helix-loop-helix protein |
| | | | | | 20)(bHLHe20) |
| 1177 | HLA-B*35:01_YPAPLESLDY | 1002 | PRA10 | ENSG00000187545 | PRAME family member 10 |
| 1178 | HLA-C*02:02_AEVGGVFASL | 1003 | FGF16 | ENSG00000196468 | Fibroblast growth factor 16 (FGF-16) |
| 1179 | HLA-B*44:03_AEAITAPLF | 827 | RHF2B | ENSG00000203989 | Rhox homeobox family member 2B |
| 1180 | HLA-C*02:02_AEAITAPLF | 827 | RHF2B | ENSG00000203989 | Rhox homeobox family member 2B |
| 1181 | HLA-A*31:01_AVQIWFENR | 828 | RHF2B | ENSG00000203989 | Rhox homeobox family member 2B |
| 1182 | HLA-A*02:07_IVPSFTFPNV | 829 | RHF2B | ENSG00000203989 | Rhox homeobox family member 2B |
| 1183 | HLA-A*01:01_QSEKEPGQQY | 830 | RHF2B | ENSG00000203989 | Rhox homeobox family member 2B |
| 1184 | HLA-B*44:03_SEKEPGQQY | 831 | RHF2B | ENSG00000203989 | Rhox homeobox family member 2B |
| 1185 | HLA-C*02:02_SEKEPGQQY | 831 | RHF2B | ENSG00000203989 | Rhox homeobox family member 2B |
| 1186 | HLA-A*03:01_GVLNQPGILK | 1004 | A0A1B0GUY1 | ENSG00000248109 | Uncharacterized protein |
| 1187 | HLA-B*35:01_LPAALSSEQM | 1005 | NMUR2 | ENSG00000132911 | Neuromedin-U receptor 2 (NMU-R2)(G-protein |
| | | | | | coupled receptor FM-4)(G-protein coupled |
| | | | | | receptor TGR-1) |
| 1188 | HLA-B*44:03_EEAQLAIRI | 1006 | RBM46 | ENSG00000151962 | Probable RNA-binding protein 46 (Cancer/testis |
| | | | | | antigen 68)(CT68)(RNA-binding motif protein |
| | | | | | 46) |
| 1189 | HLA-B*18:01_DEADFSEHTTY | 1007 | Q5T1N2 | ENSG00000162641 | Protein AKNAD1 |
| 1190 | HLA-A*03:01_KSYQGQSPQK | 859 | Q5T1N2 | ENSG00000162641 | Protein AKNAD 1 |
| 1191 | HLA-B*18:01_SEKIVYVY | 1008 | SSX3 | ENSG00000165584 | Protein SSX3 (Cancer/testis antigen 5.3)(CT5.3) |
| 1192 | HLA-A*26:01_ETPTSRQLSEY | 1009 | PERL | ENSG00000167419 | Lactoperoxidase (LPO)(EC 1.11.1.7)(Salivary |
| | | | | | peroxidase)(SPO) |
| 1193 | HLA-B*51:01_NPYFQNKVI | 965 | C9JPU3 | ENSG00000168757 | Testis-specific Y-encoded protein 2 |
| 1194 | HLA-B*44:03_QEINTKSAF | 744 | GPC6A | ENSG00000173612 | G-protein coupled receptor family C group 6 |
| | | | | | member A (hGPRC6A)(G-protein coupled |
| | | | | | receptor GPCR33)(hGPCR33) |
| 1195 | HLA-A*11:01_ASEDNLTSLLK | 1010 | KCNH7 | ENSG00000184611 | Potassium voltage-gated channel subfamily H |
| | | | | | member 7 (Ether-a-go-go-related gene potassium |
| | | | | | channel 3)(ERG-3)(Eag-related protein 3) |
| | | | | | (Ether-a-go-go-related protein 3)(hERG-3) |
| | | | | | (Voltage-gated potassium channel subunit |
| | | | | | Kv11.3) |
| 1196 | HLA-A*02:01_FLYHDSTDIGL | 1011 | ADAM7 | ENSG00000069206 | Disintegrin and metalloproteinase domain- |
| | | | | | containing protein 7 (ADAM 7)(Sperm |
| | | | | | maturation-related glycoprotein GP-83) |
| 1197 | HLA-B*51:01_MPMSEVSQV | 1012 | U3KQD4 | ENSG00000105549 | Testicular haploid-expressed gene protein (Theg |
| | | | | | homolog (Mouse), isoform CRA_a) |
| 1198 | HLA-A*02:01_TLTITVPAV | 1013 | U3KQD4 | ENSG00000105549 | Testicular haploid-expressed gene protein (Theg |
| | | | | | homolog (Mouse), isoform CRA_a) |
| 1199 | HLA-A*30:02_ISNPLLIGRY | 1014 | TTLL2 | ENSG00000120440 | Probable tubulin polyglutamylase TTLL2 (EC 6.- |
| | | | | | .-.-)(Testis-specific protein NYD-TSPG) |
| | | | | | (Tubulin--tyro sine ligase-like protein 2) |
| 1200 | HLA-A*24:02_KYISNPLLI | 1015 | TTLL2 | ENSG00000120440 | Probable tubulin polyglutamylase TTLL2 (EC 6.- |
| | | | | | .-.-)(Testis-specific protein NYD-TSPG) |
| | | | | | (Tubulin--tyro sine ligase-like protein 2) |
| 1201 | HLA-A*24:02_VYQEGLVRF | 1016 | TTLL2 | ENSG00000120440 | Probable tubulin polyglutamylase TTLL2 (EC 6.- |
| | | | | | .-.-)(Testis-specific protein NYD-TSPG) |
| | | | | | (Tubulin--tyro sine ligase-like protein 2) |
| 1202 | HLA-C*16:01_AAAAAGLAY | 646 | BHE23 | ENSG00000125533 | Class E basic helix-loop-helix protein 23 |
| | | | | | (bHLHe23)(Class B basic helix-loop-helix |
| | | | | | protein 4)(bHLHb4) |
| 1203 | HLA-C*16:01_AAAAAAATY | 372 | NKX24 | ENSG00000125816 | Homeobox protein Nkx-2.4 (Homeobox protein |
| | | | | | NK-2 homolog D) |
| 1204 | HLA-A*29:02_GVLGANLLY | 1017 | NKX24 | ENSG00000125816 | Homeobox protein Nkx-2.4 (Homeobox protein |
| | | | | | NK-2 homolog D) |
| 1205 | HLA-C*02:02_IEAGTSESY | 276 | SUN3 | ENSG00000164744 | SUN domain-containing protein 3 (Sad1/unc-84 |
| | | | | | domain-containing protein 1) |
| 1206 | HLA-A*11:01_TTYTGSYRK | 1018 | CD051 | ENSG00000237136 | Uncharacterized protein C4orf51 |
| 1207 | HLA-A*02:07_LLDGQWHHI | 1019 | PTX4 | ENSG00000251692 | Pentraxin-4 |
| 1208 | HLA-B*35:01_LPNENFQSLY | 1020 | RNF17 | ENSG00000132972 | RING finger protein 17 (Tudor domain- |
| | | | | | containing protein 4) |
| 1209 | HLA-B*44:02_EEDVWVIIHLY | 761 | PDCL2 | ENSG00000163440 | Phosducin-like protein 2 |
| 1210 | HLA-B*35:01_TATSTGQLY | 1021 | CP2AD | ENSG00000197838 | Cytochrome P450 2A13 (EC 1.14.14.1) |
| | | | | | (CYPIIA13) |
| 1211 | HLA-B*44:02_SEKEPGQQY | 831 | RHF2B | ENSG00000203989 | Rhox homeobox family member 2B |
| 1212 | HLA-B*51:01_MPAAAALI | 1022 | DYTN | ENSG00000232125 | Dystrotelin |
| 1213 | HLA-A*24:02_KYLYVTSSF | 1023 | MORC1 | ENSG00000114487 | MORC family CW-type zinc finger protein 1 |
| | | | | | (Cancer/testis antigen 33)(CT33) |
| 1214 | HLA-A*03:01_GTMKIHILQK | 112 | V9GZ46 | ENSG00000124092 | Transcriptional repressor CTCFL |
| 1215 | HLA-A*03:01_SSYVPIFEK | 620 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 1216 | HLA-B*35:01_MPFAISTSL | 1024 | S7A13 | ENSG00000164893 | Solute carrier family 7 member 13 (Sodium- |
| | | | | | independent aspartate/glutamate transporter 1) |
| | | | | | (X-amino acid transporter 2) |
| 1217 | HLA-B*44:03_EEASVYSQW | 1025 | WDR87 | ENSG00000171804 | WD repeat-containing protein 87 (Testis |
| | | | | | development protein NYD-SP11) |
| 1218 | HLA-C*02:02_EEASVYSQW | 1025 | WDR87 | ENSG00000171804 | WD repeat-containing protein 87 (Testis |
| | | | | | development protein NYD-SP11) |
| 1219 | HLA-A*02:01_RLWPEGTPIYL | 1026 | WDR87 | ENSG00000171804 | WD repeat-containing protein 87 (Testis |
| | | | | | development protein NYD-SP11) |
| 1220 | HLA-A*02:07_VLDGVKVQL | 1027 | WDR87 | ENSG00000171804 | WD repeat-containing protein 87 (Testis |
| | | | | | development protein NYD-SP11) |
| 1221 | HLA-A*11:01_VTQEVIRFIK | 1028 | WDR87 | ENSG00000171804 | WD repeat-containing protein 87 (Testis |
| | | | | | development protein NYD-SP11) |
| 1222 | HLA-A*02:01_FLSDNTIEV | 1029 | GPC6A | ENSG00000173612 | G-protein coupled receptor family C group 6 |
| | | | | | member A (hGPRC6A)(G-protein coupled |
| | | | | | receptor GPCR33)(hGPCR33) |
| 1223 | HLA-A*24:02_NYNEAKFITF | 1030 | GPC6A | ENSG00000173612 | G-protein coupled receptor family C group 6 |
| | | | | | member A (hGPRC6A)(G-protein coupled |
| | | | | | receptor GPCR33)(hGPCR33) |
| 1224 | HLA-B*18:01_VEWEVHGM | 1031 | ACHA9 | ENSG00000174343 | Neuronal acetylcholine receptor subunit alpha-9 |
| | | | | | (Nicotinic acetylcholine receptor subunit alpha-9) |
| | | | | | (NACHR alpha-9) |
| 1225 | HLA-B*44:02_KEGEPVEFIF | 241 | LN28B | ENSG00000187772 | Protein lin-28 homolog B (Lin-28B) |
| 1226 | HLA-B*44:02_AESPLEVPQSF | 626 | MAGBG | ENSG00000189023 | Melanoma-associated antigen B16 (MAGE-B16 |
| | | | | | antigen) |
| 1227 | HLA-A*31:01_TTLGIDYVNPR | 1032 | GNAT3 | ENSG00000214415 | Guanine nucleotide-binding protein G(t) subunit |
| | | | | | alpha-3 (Gustducin alpha-3 chain) |
| 1228 | HLA-A*03:01_HVSPSPLIY | 432 | TIFAB | ENSG00000255833 | TRAP-interacting protein with FHA domain- |
| | | | | | containing protein B (TWA-like protein) |
| 1229 | HLA-A*24:02_NYTDFSGSSF | 1033 | TSN16 | ENSG00000130167 | Tetraspanin-16 (Tspan-16)(Tetraspanin TM4-B) |
| | | | | | (Transmembrane 4 supetfamily member 16) |
| 1230 | HLA-B*18:01_YEAFLSPEY | 1034 | PPAT | ENSG00000142513 | Testicular acid phosphatase (EC 3.1.3.2) |
| 1231 | HLA-A*02:01_ALAAVDIAV | 1035 | PO4F1 | ENSG00000152192 | POU domain, class 4, transcription factor 1 |
| | | | | | (Brain-specific homeobox/POU domain protein |
| | | | | | 3A)(Brain-3A)(Brn-3A)(Homeobox/POU |
| | | | | | domain protein RDC-1)(Oct-T1) |
| 1232 | HLA-B*18:01_DENLIYVI | 1036 | SL9C1 | ENSG00000172139 | Sodium/hydrogen exchanger 10 (Na(+)/H(+) |
| | | | | | exchanger 10)(NHE-10)(Solute carrier family 9 |
| | | | | | member 10)(Solute carrier family 9 member Cl) |
| | | | | | (Sperm-specific Na(+)/H(+) exchanger)(sNHE) |
| 1233 | HLA-A*31:01_QVKIWFQNR | 110 | NKX26 | ENSG00000180053 | Homeobox protein Nkx-2.6 (Homeobox protein |
| | | | | | NK-2 homolog F) |
| 1234 | HLA-B*44:02_GEYPDYQQW | 604 | CRGB | ENSG00000182187 | Gamma-crystallin B (Gamma-B-crystallin) |
| | | | | | (Gamma-crystallin 1-2) |
| 1235 | HLA-A*11:01_AVHNEDKLMAK | 1037 | D7UEQ8 | ENSG00000183206 | POTE ankyrin domain family member C |
| 1236 | HLA-A*31:01_AVKKPFDLR | 1038 | D7UEQ8 | ENSG00000183206 | POTE ankyrin domain family member C |
| 1237 | HLA-B*18:01_DEYGNTTLHY | 1039 | D7UEQ8 | ENSG00000183206 | POTE ankyrin domain family member C |
| 1238 | HLA-A*11:01_ISQDEILTNK | 1040 | D7UEQ8 | ENSG00000183206 | POTE ankyrin domain family member C |
| 1239 | HLA-B*18:01_LEEEVTTY | 1041 | RTP2 | ENSG00000198471 | Receptor-transporting protein 2 (3CxxC-type zinc |
| | | | | | finger protein 2) |
| 1240 | HLA-A*02:07_LVDNLITSL | 1042 | RTP2 | ENSG00000198471 | Receptor-transporting protein 2 (3CxxC-type zinc |
| | | | | | finger protein 2) |
| 1241 | HLA-C*16:01_AAVSDPRVY | 1043 | A0A0U1RRI6 | ENSG00000224109 | Centromere protein V-like 3 |
| 1242 | HLA-A*11:01_ATLENLLSH | 1044 | PRAM4 | ENSG00000243073 | PRAME family member 4 |
| 1243 | HLA-A*30:02_TVNKLNHAY | 1045 | A0A1B0GW35 | ENSG00000250821 | HCG2040572 |
| 1244 | HLA-A*01:01_QSEQSSVRY | 1046 | SACA1 | ENSG00000118434 | Sperm acrosome membrane-associated protein 1 |
| | | | | | (Sperm acrosomal membrane-associated protein |
| | | | | | 32) |
| 1245 | HLA-A*29:02_ILSPIEETY | 1047 | NKX24 | ENSG00000125816 | Homeobox protein Nkx-2.4 (Homeobox protein |
| | | | | | NK-2 homolog D) |
| 1246 | HLA-A*02:01_GLLSDLVYL | 1048 | LRC52 | ENSG00000162763 | Leucine-rich repeat-containing protein 52 (BK |
| | | | | | channel auxiliary gamma subunit LRRC52) |
| 1247 | HLA-A*11:01_GTNGFQLLR | 1049 | A0A0B41218 | ENSG00000164871, | Sperm-associated antigen 11A (Sperm-associated |
| | | | | ENSG00000178287 | antigen 11B)(Fragment) |
| 1248 | HLA-A*11:01_GTNGFQLLR | 1049 | SG11B | ENSG00000164871 | Sperm-associated antigen 11B (Human |
| | | | | | epididymis-specific protein 2)(He2)(Protein |
| | | | | | EP2)(Sperm antigen HE2) |
| 1249 | HLA-A*02:07_LLPPRTPPYQV | 1050 | SG11B | ENSG00000164871 | Sperm-associated antigen 11B (Human |
| | | | | | epididymis-specific protein 2)(He2)(Protein |
| | | | | | EP2)(Sperm antigen HE2) |
| 1250 | HLA-B*51:01_VPPGIRNTI | 1051 | SG11B | ENSG00000164871 | Sperm-associated antigen 11B (Human |
| | | | | | epididymis-specific protein 2)(He2)(Protein |
| | | | | | EP2)(Sperm antigen HE2) |
| 1251 | HLA-B*35:01_VPSYPGNTY | 1052 | PRR27 | ENSG00000187533 | Proline-rich protein 27 |
| 1252 | HLA-A*30:02_RLLPPVSGGY | 1053 | CP26C | ENSG00000187553 | Cytochrome P450 26C1 (EC 1.14.-.-) |
| 1253 | HLA-B*51:01_YPISPKVI | 1054 | M4A18 | ENSG00000214782 | Membrane-spanning 4-domains subfamily A |
| | | | | | member 18 |
| 1254 | HLA-A*11:01_GVLNQPGILK | 1004 | A0A1B0GUY1 | ENSG00000248109 | Uncharacterized protein |
| 1255 | HLA-C*02:02_AAAAAAATY | 372 | NKX24 | ENSG00000125816 | Homeobox protein Nkx-2.4 (Homeobox protein |
| | | | | | NK-2 homolog D) |
| 1256 | HLA-B*51:01_DAWLFGALV | 1055 | GALR3 | ENSG00000128310 | Galanin receptor type 3 (GAL3-R)(GALR-3) |
| 1257 | HLA-A*26:01_DVATFAAGY | 1056 | GALR3 | ENSG00000128310 | Galanin receptor type 3 (GAL3-R)(GALR-3) |
| 1258 | HLA-A*02:01_QLWGHTIQV | 1057 | RBM46 | ENSG00000151962 | Probable RNA-binding protein 46 (Cancer/testis |
| | | | | | antigen 68)(CT68)(RNA-binding motif protein |
| | | | | | 46) |
| 1259 | HLA-A*02:01_SLSPVSATL | 1058 | RBM46 | ENSG00000151962 | Probable RNA-binding protein 46 (Cancer/testis |
| | | | | | antigen 68)(CT68)(RNA-binding motif protein |
| | | | | | 46) |
| 1260 | HLA-B*08:01_DLVQRNVSI | 1059 | LDH6A | ENSG00000166800 | L-lactate dehydrogenase A-like 6A (EC 1.1.1.27) |
| 1261 | HLA-A*11:01_GSIDDGNFQK | 1060 | ADA18 | ENSG00000168619 | Disintegrin and metalloproteinase domain- |
| | | | | | containing protein 18 (ADAM 18) |
| | | | | | (Transmembrane metalloproteinase-like, |
| | | | | | disintegrin-like, and cysteine-rich protein III) |
| | | | | | (tMDC III) |
| 1262 | HLA-A*11:01_KAMAHLIQK | 1061 | GPC6A | ENSG00000173612 | G-protein coupled receptor family C group 6 |
| | | | | | member A (hGPRC6A)(G-protein coupled |
| | | | | | receptor GPCR33)(hGPCR33) |
| 1263 | HLA-A*03:01_RLSAESKDLLK | 1062 | OLIG3 | ENSG00000177468 | Oligodendrocyte transcription factor 3 (01ig03) |
| | | | | | (Class B basic helix-loop-helix protein 7) |
| | | | | | (bHLHb7)(Class E basic helix-loop-helix protein |
| | | | | | 20)(bHLHe20) |
| 1264 | HLA-B*35:01_QPLLHVTAY | 1063 | CH086 | ENSG00000196166 | Uncharacterized protein C8orf86 |
| 1265 | HLA-A*02:07_ALDNIVTQF | 1064 | CD022 | ENSG00000197826 | Uncharacterized protein C4orf22 |
| 1266 | HLA-A*02:01_FLDSQITTV | 1065 | CD022 | ENSG00000197826 | Uncharacterized protein C4orf22 |
| 1267 | HLA-A*02:07_FLDSQITTV | 1065 | CD022 | ENSG00000197826 | Uncharacterized protein C4orf22 |
| 1268 | HLA-A*30:02_SSSGLSSSY | 1066 | HORN | ENSG00000197915 | Hornerin |
| 1269 | HLA-B*51:01_LPPLIPSL | 1067 | CP096 | ENSG00000205832 | Uncharacterized protein Cl6orf96 |
| 1270 | HLA-B*51:01_DALKQSLVV | 1068 | PCDG8 | ENSG00000253767 | Protocadherin gamma-A8 (PCDH-gamma-A8) |
| 1271 | HLA-B*44:03_EEVQAFLQTY | 1069 | PCDG8 | ENSG00000253767 | Protocadherin gamma-A8 (PCDH-gamma-A8) |
| 1272 | HLA-A*30:02_GTMGLSARY | 1070 | PCDG8 | ENSG00000253767 | Protocadherin gamma-A8 (PCDH-gamma-A8) |
| 1273 | HLA-A*02:01_YLVTKVVAV | 1071 | PCDG8 | ENSG00000253767 | Protocadherin gamma-A8 (PCDH-gamma-A8) |
| 1274 | HLA-B*44:03_DETEIRSFF | 589 | DAZL | ENSG00000092345 | Deleted in azoospermia-like (DAZ homolog) |
| | | | | | (DAZ-like autosomal)(Deleted in azoospermia- |
| | | | | | like 1)(SPGY-like-autosomal) |
| 1275 | HLA-B*51:01_IPPSFVKMV | 1072 | PP13 | ENSG00000105198 | Galactoside-binding soluble lectin 13 (Galectin- |
| | | | | | 13)(Ga1-13)(Placental tissue protein 13)(PP13) |
| | | | | | (Placental protein 13) |
| 1276 | HLA-A*03:01_RLGPLTTSH | 1073 | APOL5 | ENSG00000128313 | Apolipoprotein L5 (Apolipoprotein L-V)(ApoL- |
| | | | | | V) |
| 1277 | HLA-A*01:01_VTDTLVEVLLY | 1074 | RNF17 | ENSG00000132972 | RING finger protein 17 (Tudor domain- |
| | | | | | containing protein 4) |
| 1278 | HLA-A*24:02_EYAGNFQGI | 1075 | FATE1 | ENSG00000147378 | Fetal and adult testis-expressed transcript protein |
| | | | | | (Cancer/testis antigen 43)(CT43)(Tumor antigen |
| | | | | | BJ-HCC-2) |
| 1279 | HLA-B*51:01_DSYVFVNTL | 1076 | MAGC1 | ENSG00000155495 | Melanoma-associated antigen C1 (Cancer/testis |
| | | | | | antigen 7.1)(CT7.1)(MAGE-C1 antigen) |
| 1280 | HLA-A*24:02_LYLPEPFKI | 1077 | CC049 | ENSG00000163632 | Putative uncharacterized protein C3orf49 |
| 1281 | HLA-A*02:07_YLPEPFKIA | 1078 | CC049 | ENSG00000163632 | Putative uncharacterized protein C3orf49 |
| 1282 | HLA-B*46:01_MVILGVTSF | 1079 | ZPLD1 | ENSG00000170044 | Zona pellucida-like domain-containing protein 1 |
| | | | | | (ZP domain-containing protein 1) |
| 1283 | HLA-B*08:01_MPAVKNVI | 1080 | ACHA9 | ENSG00000174343 | Neuronal acetylcholine receptor subunit alpha-9 |
| | | | | | (Nicotinic acetylcholine receptor subunit alpha-9) |
| | | | | | (NACHR alpha-9) |
| 1284 | HLA-A*29:02_LLSLVGFVY | 1081 | NKAI3 | ENSG00000185942 | Sodium/potassium-transporting ATPase subunit |
| | | | | | beta-1-interacting protein 3 (Na(+)/K(+)- |
| | | | | | transporting ATPase subunit beta-1-interacting |
| | | | | | protein 3)(Protein FAM77D) |
| 1285 | HLA-C*02:02_AELTGGSEW | 1082 | C9J420 | ENSG00000186038 | 5-hydroxytryptamine receptor 3E (Fragment) |
| 1286 | HLA-A*29:02_IFIIITDLSLY | 1083 | M4A18 | ENSG00000214782 | Membrane-spanning 4-domains subfamily A |
| | | | | | member 18 |
| 1287 | HLA-C*02:02_SAINPVLYY | 1084 | M4A18 | ENSG00000214782 | Membrane-spanning 4-domains subfamily A |
| | | | | | member 18 |
| 1288 | HLA-A*30:02_RVAELINASY | 1085 | DC8L1 | ENSG00000226372 | DDB1-and CUL4-associated factor 8-like protein |
| | | | | | 1 (WD repeat-containing protein 42B) |
| 1289 | HLA-B*35:01_TAASSDIEM | 1086 | DC8L1 | ENSG00000226372 | DDB1-and CUL4-associated factor 8-like protein |
| | | | | | 1 (WD repeat-containing protein 42B) |
| 1290 | HLA-B*18:01_NEYFSTKY | 1087 | A14EL | ENSG00000268223 | ARL14 effector protein-like |
| 1291 | HLA-A*33:01_DTNIIANR | 1088 | ADAM7 | ENSG00000069206 | Disintegrin and metalloproteinase domain- |
| | | | | | containing protein 7 (ADAM 7)(Sperm |
| | | | | | maturation-related glycoprotein GP-83) |
| 1292 | HLA-A*11:01_QVASQEDILLK | 1089 | SMC1B | ENSG00000077935 | Structural maintenance of chromosomes protein |
| | | | | | 1B (SMC protein 1B)(SMC-1-beta)(SMC-1B) |
| 1293 | HLA-B*18:01_IEAIRAEY | 818 | TRI51 | ENSG00000124900 | Tripartite motif-containing protein 51 (SPRY |
| | | | | | domain-containing protein 5) |
| 1294 | HLA-A*01:01_VSDSTYYSSFY | 1090 | DMRT1 | ENSG00000137090 | Double sex-and mab-3-related transcription factor |
| | | | | | 1 (DM domain expressed in testis protein 1) |
| 1295 | HLA-A*02:01_FLGPATAHL | 1091 | X6R6V8 | ENSG00000143552 | Nuclear pore membrane glycoprotein 210-like |
| 1296 | HLA-A*24:02_IYSVRVVNF | 1092 | X6R6V8 | ENSG00000143552 | Nuclear pore membrane glycoprotein 210-like |
| 1297 | HLA-A*31:01_RVLPWADRTAR | 1093 | LGSN | ENSG00000146166 | Lengsin (Glutamate-ammonia ligase domain- |
| | | | | | containing protein 1)(Lens glutamine synthase- |
| | | | | | like) |
| 1298 | HLA-B*35:01_NPLGDIASL | 1094 | CG072 | ENSG00000164500 | Uncharacterized protein C7oif72 |
| 1299 | HLA-B*51:01_FPYVVLVI | 1095 | SC6A5 | ENSG00000165970 | Sodium-and chloride-dependent glycine |
| | | | | | transporter 2 (GlyT-2)(GlyT2)(Solute carrier |
| | | | | | family 6 member 5) |
| 1300 | HLA-A*02:07_GLPIFFLEV | 1096 | SC6A5 | ENSG00000165970 | Sodium-and chloride-dependent glycine |
| | | | | | transporter 2 (GlyT-2)(GlyT2)(Solute carrier |
| | | | | | family 6 member 5) |
| 1301 | HLA-B*51:01_TAYPNVTMV | 1097 | SC6A5 | ENSG00000165970 | Sodium-and chloride-dependent glycine |
| | | | | | transporter 2 (GlyT-2)(GlyT2)(Solute carrier |
| | | | | | family 6 member 5) |
| 1302 | HLA-B*51:01_WAFVTPTI | 1098 | SC6A5 | ENSG00000165970 | Sodium-and chloride-dependent glycine |
| | | | | | transporter 2 (GlyT-2)(GlyT2)(Solute carrier |
| | | | | | family 6 member 5) |
| 1303 | HLA-A*11:01_QTLEELNTVLK | 1099 | PERL | ENSG00000167419 | Lactoperoxidase (LPO)(EC 1.11.1.7)(Salivary |
| | | | | | peroxidase)(SPO) |
| 1304 | HLA-A*01:01_FTSSQVQRY | 998 | SL9C1 | ENSG00000172139 | Sodium/hydrogen exchanger 10 (Na(+)/H(+) |
| | | | | | exchanger 10)(NHE-10)(Solute carrier family 9 |
| | | | | | member 10)(Solute carrier family 9 member Cl) |
| | | | | | (Sperm-specific Na(+)/H(+) exchanger)(sNHE) |
| 1305 | HLA-C*02:02_AEAELTGGSEW | 843 | C9J420 | ENSG00000186038 | 5-hydroxytryptamine receptor 3E (Fragment) |
| 1306 | HLA-A*29:02_RNLPPPLYY | 1100 | PRR27 | ENSG00000187533 | Proline-rich protein 27 |
| 1307 | HLA-A*02:01_KVLEFVAKV | 986 | MAGBG | ENSG00000189023 | Melanoma-associated antigen B16 (MAGE-B16 |
| | | | | | antigen) |
| 1308 | HLA-B*35:01_FPTGAILTL | 1101 | PTX4 | ENSG00000251692 | Pentraxin-4 |
| 1309 | HLA-B*44:03_NEQESLLSRY | 1102 | TULP2 | ENSG00000104804 | Tubby-related protein 2 (Cancer/testis antigen 65) |
| | | | | | (CT65)(Tubby-like protein 2) |
| 1310 | HLA-B*51:01_DALLAQKV | 1103 | PRA12 | ENSG00000116726 | PRAME family member 12 |
| 1311 | HLA-B*44:03_SESDLKHLSW | 1104 | PRA12 | ENSG00000116726 | PRAME family member 12 |
| 1312 | HLA-A*11:01_ASQVPSHSPK | 1105 | ZSC10 | ENSG00000130182 | Zinc finger and SCAN domain-containing protein |
| | | | | | 10 (Zinc finger protein 206) |
| 1313 | HLA-B*27:02_LRNQLDQQF | 1106 | F71F1 | ENSG00000135248 | Protein FAM71F1 (Protein FAM137A)(Testis |
| | | | | | development protein NYD-SP18) |
| 1314 | HLA-A*02:01_ALADFMLSL | 1107 | GPR32 | ENSG00000142511 | Probable G-protein coupled receptor 32 |
| 1315 | HLA-A*29:02_YITFVFLSY | 1108 | GPR32 | ENSG00000142511 | Probable G-protein coupled receptor 32 |
| 1316 | HLA-A*11:01_STSTVPLAH | 1109 | PO4F1 | ENSG00000152192 | POU domain, class 4, transcription factor 1 |
| | | | | | (Brain-specific homeobox/POU domain protein |
| | | | | | 3A)(Brain-3A)(Brn-3A)(Homeobox/POU |
| | | | | | domain protein RDC-1)(Oct-T1) |
| 1317 | HLA-B*44:02_GEYPDYQQW | 604 | CRGC | ENSG00000163254 | Gamma-crystallin C (Gamma-C-crystallin) |
| | | | | | (Gamma-crystallin 2-1)(Gamma-crystallin 3) |
| 1318 | HLA-A*31:01_AVAVWVHVR | 1110 | CG033 | ENSG00000170279 | Uncharacterized protein C7orf33 |
| 1319 | HLA-A*29:02_SYLDLLTLSY | 1111 | CG033 | ENSG00000170279 | Uncharacterized protein C7orf33 |
| 1320 | HLA-A*01:01_YLDLLTLSY | 1112 | CG033 | ENSG00000170279 | Uncharacterized protein C7orf33 |
| 1321 | HLA-A*24:02_NYAPPVVKF | 1113 | V9GZ31 | ENSG00000177414 | Ubiquitin-conjugating enzyme E2 U (Fragment) |
| 1322 | HLA-A*02:07_GLPWRFEEL | 1114 | TEX19 | ENSG00000182459 | Testis-expressed protein 19 |
| 1323 | HLA-A*26:01_EVIAGLERF | 1115 | H9KVA5 | ENSG00000187959 | Putative cleavage and polyadenylation-specificity |
| | | | | | factor subunit 4-like protein |
| 1324 | HLA-B*35:01_FAFEKDVEM | 1116 | H9KVA5 | ENSG00000187959 | Putative cleavage and polyadenylation-specificity |
| | | | | | factor subunit 4-like protein |
| 1325 | HLA-A*26:01_EVASAVSAFGY | 1117 | 1A1L2 | ENSG00000205126 | Probable inactive 1-amino cyclopropane-1- |
| | | | | | carboxylate synthase-like protein 2 (ACC |
| | | | | | synthase-like protein 2) |
| 1326 | HLA-A*29:02_IFSAINPVLY | 1118 | M4A18 | ENSG00000214782 | Membrane-spanning 4-domains subfamily A |
| | | | | | member 18 |
| 1327 | HLA-A*29:02_SAINPVLYY | 1084 | M4A18 | ENSG00000214782 | Membrane-spanning 4-domains subfamily A |
| | | | | | member 18 |
| 1328 | HLA-A*26:01_EVYDEDPFAY | 1119 | E5RK72 | ENSG00000215262 | Potassium channel subfamily U member 1 |
| 1329 | HLA-B*18:01_TENIVAVM | 1120 | RN148 | ENSG00000235631 | RING finger protein 148 |
| 1330 | HLA-B*18:01_NENSLVSF | 1121 | ERVV2 | ENSG00000268964 | Endogenous retrovirus group V member 2 Env |
| | | | | | polyprotein (HERV-V_19q13.41 provirus |
| | | | | | ancestral Env polyprotein 2) |
| 1331 | HLA-A*02:07_VQDDTLHNV | 1122 | TRPC7 | ENSG00000069018 | Short transient receptor potential channel 7 |
| | | | | | (TrpC7)(Transient receptor protein 7)(TRP-7) |
| | | | | | (hTRP7) |
| 1332 | HLA-B*46:01_YSHVQGISY | 1123 | ADAM7 | ENSG00000069206 | Disintegrin and metalloproteinase domain- |
| | | | | | containing protein 7 (ADAM 7)(Sperm |
| | | | | | maturation-related glycoprotein GP-83) |
| 1333 | HLA-A*26:01_ESAGVMSVY | 1124 | CCG5 | ENSG00000075429 | Voltage-dependent calcium channel gamma-5 |
| | | | | | subunit (Neuronal voltage-gated calcium channel |
| | | | | | gamma-5 subunit)(Transmembrane AMPAR |
| | | | | | regulatory protein gamma-5)(TARP gamma-5) |
| 1334 | HLA-B*35:01_EPYLEGISY | 1125 | SMC1B | ENSG00000077935 | Structural maintenance of chromosomes protein |
| | | | | | 1B (SMC protein 1B)(SMC-1-beta)(SMC-1B) |
| 1335 | HLA-C*16:01_SASGPGLAF | 1126 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 1336 | HLA-A*02:07_ILDVIGVKV | 1127 | ADA29 | ENSG00000168594 | Disintegrin and metalloproteinase domain- |
| | | | | | containing protein 29 (ADAM 29)(Cancer/testis |
| | | | | | antigen 73)(CT73) |
| 1337 | HLA-A*11:01_STLTVDIANK | 1128 | CTSRD | ENSG00000174898 | Cation channel sperm-associated protein subunit |
| | | | | | delta (CatSper-delta)(CatSperdelta) |
| | | | | | (Transmembrane protein 146) |
| 1338 | HLA-C*16:01_VVTGHQQSF | 1129 | DC4L2 | ENSG00000176566 | DDB1-and CUL4-associated factor 4-like protein |
| | | | | | 2 (WD repeat-containing protein 21C) |
| 1339 | HLA-B*44:03_QEVVGELVAKF | 1130 | TFDP3 | ENSG00000183434 | Transcription factor Dp family member 3 |
| | | | | | (Cancer/testis antigen 30)(CT30)(Hepatocellular |
| | | | | | carcinoma-associated antigen 661) |
| 1340 | HLA-B*35:01_LPGPQQQAF | 1131 | CP2AD | ENSG00000197838 | Cytochrome P450 2A13 (EC 1.14.14.1) |
| | | | | | (CYPIIA13) |
| 1341 | HLA-A*02:01_YLLEKIPLV | 806 | NTM1B | ENSG00000203740 | Alpha N-terminal protein methyltransferase 1B |
| | | | | | (EC 2.1.1.299)(Methyltransferase-like protein |
| | | | | | 11B)(X-Pro-Lys N-terminal protein |
| | | | | | methyltransferase 1B)(NTM1B) |
| 1342 | HLA-B*44:02_AENESVIIRL | 1132 | TDR15 | ENSG00000218819 | Tudor domain-containing protein 15 |
| 1343 | HLA-B*44:03_AENESVIIRL | 1132 | TDR15 | ENSG00000218819 | Tudor domain-containing protein 15 |
| 1344 | HLA-C*02:02_AENESVIIRL | 1132 | TDR15 | ENSG00000218819 | Tudor domain-containing protein 15 |
| 1345 | HLA-A*11:01_AILLQVIAK | 1133 | TDR15 | ENSG00000218819 | Tudor domain-containing protein 15 |
| 1346 | HLA-A*02:01_ALLGQVVYA | 1134 | TDR15 | ENSG00000218819 | Tudor domain-containing protein 15 |
| 1347 | HLA-A*26:01_AVITEINGY | 1135 | TDR15 | ENSG00000218819 | Tudor domain-containing protein 15 |
| 1348 | HLA-B*51:01_DARIFQLSI | 1136 | TDR15 | ENSG00000218819 | Tudor domain-containing protein 15 |
| 1349 | HLA-B*51:01_DPYEIGQTA | 1137 | TDR15 | ENSG00000218819 | Tudor domain-containing protein 15 |
| 1350 | HLA-A*26:01_DVISLMLQAGY | 1138 | TDR15 | ENSG00000218819 | Tudor domain-containing protein 15 |
| 1351 | HLA-B*44:03_EEVYISHIY | 1139 | TDR15 | ENSG00000218819 | Tudor domain-containing protein 15 |
| 1352 | HLA-A*01:01_EVDIVEVDY | 1140 | TDR15 | ENSG00000218819 | Tudor domain-containing protein 15 |
| 1353 | HLA-B*18:01_FEVEFIDY | 1141 | TDR15 | ENSG00000218819 | Tudor domain-containing protein 15 |
| 1354 | HLA-A*11:01_GSMNSNQQLFK | 1142 | TDR15 | ENSG00000218819 | Tudor domain-containing protein 15 |
| 1355 | HLA-B*35:01_IPTDSSSEF | 1143 | TDR15 | ENSG00000218819 | Tudor domain-containing protein 15 |
| 1356 | HLA-B*44:03_KEVDIVFVDY | 1144 | TDR15 | ENSG00000218819 | Tudor domain-containing protein 15 |
| 1357 | HLA-A*11:01_KTVDYFTSK | 1145 | TDR15 | ENSG00000218819 | Tudor domain-containing protein 15 |
| 1358 | HLA-A*24:02_KYVDDKVLVF | 1146 | TDR15 | ENSG00000218819 | Tudor domain-containing protein 15 |
| 1359 | HLA-B*51:01_LPKSLAVNI | 1147 | TDR15 | ENSG00000218819 | Tudor domain-containing protein 15 |
| 1360 | HLA-A*01:01_PTDSSSEFQVY | 1148 | TDR15 | ENSG00000218819 | Tudor domain-containing protein 15 |
| 1361 | HLA-B*35:01_QALLGQVVY | 1149 | TDR15 | ENSG00000218819 | Tudor domain-containing protein 15 |
| 1362 | HLA-A*11:01_QTQESTVNSK | 1150 | TDR15 | ENSG00000218819 | Tudor domain-containing protein 15 |
| 1363 | HLA-A*24:02_QYITLSETF | 1151 | TDR15 | ENSG00000218819 | Tudor domain-containing protein 15 |
| 1364 | HLA-A*02:01_RLAEIVYNI | 1152 | TDR15 | ENSG00000218819 | Tudor domain-containing protein 15 |
| 1365 | HLA-A*31:01_SAKEFLMNR | 1153 | TDR15 | ENSG00000218819 | Tudor domain-containing protein 15 |
| 1366 | HLA-B*44:03_SEFKNPFTL | 1154 | TDR15 | ENSG00000218819 | Tudor domain-containing protein 15 |
| 1367 | HLA-C*02:02_SEFKNPFTL | 1154 | TDR15 | ENSG00000218819 | Tudor domain-containing protein 15 |
| 1368 | HLA-C*02:02_SETSVSDVNSF | 1155 | TDR15 | ENSG00000218819 | Tudor domain-containing protein 15 |
| 1369 | HLA-A*03:01_SLNKKGILK | 1156 | TDR15 | ENSG00000218819 | Tudor domain-containing protein 15 |
| 1370 | HLA-A*11:01_SVNLQNFPK | 1157 | TDR15 | ENSG00000218819 | Tudor domain-containing protein 15 |
| 1371 | HLA-A*30:02_VINKPSPVTY | 1158 | TDR15 | ENSG00000218819 | Tudor domain-containing protein 15 |
| 1372 | HLA-A*02:07_VLDKLQPSL | 1159 | TDR15 | ENSG00000218819 | Tudor domain-containing protein 15 |
| 1373 | HLA-A*29:02_YGFSFYIRY | 1160 | TDR15 | ENSG00000218819 | Tudor domain-containing protein 15 |
| 1374 | HLA-B*08:01_YINEKIKVL | 1161 | TDR15 | ENSG00000218819 | Tudor domain-containing protein 15 |
| 1375 | HLA-A*02:07_YVDDKVLVFL | 1162 | TDR15 | ENSG00000218819 | Tudor domain-containing protein 15 |
| 1376 | HLA-A*02:07_YVDDKVLVF | 1163 | TDR15 | ENSG00000218819 | Tudor domain-containing protein 15 |
| 1377 | HLA-A*31:01_ATLQPRGITR | 1164 | V9GYR9 | ENSG00000225362 | Cancer/testis antigen 62 (Fragment) |
| 1378 | HLA-A*02:01_YLHEQVKTI | 1165 | FHL17 | ENSG00000132446 | Ferritin heavy polypeptide-like 17 (Cancer/testis |
| | | | | | antigen 38)(CT38) |
| 1379 | HLA-B*18:01_QEHLVIAEM | 1166 | FATE1 | ENSG00000147378 | Fetal and adult testis-expressed transcript protein |
| | | | | | (Cancer/testis antigen 43)(CT43)(Tumor antigen |
| | | | | | BJ-HCC-2) |
| 1380 | HLA-A*31:01_NGKQIYVGR | 83 | PABP3 | ENSG00000151846 | Polyadenylate-binding protein 3 (PABP-3) |
| | | | | | (Poly(A)-binding protein 3)(Testis-specific |
| | | | | | poly(A)-binding protein) |
| 1381 | HLA-B*35:01_AAASLFEVY | 1167 | OTOP1 | ENSG00000163982 | Otopetrin-1 |
| 1382 | HLA-A*30:02_ATIAVVVVY | 1168 | OTOP1 | ENSG00000163982 | Otopetrin-1 |
| 1383 | HLA-B*35:01_HAAASLFEVY | 1169 | OTOP1 | ENSG00000163982 | Otopetrin-1 |
| 1384 | HLA-B*51:01_LPYSILAI | 1170 | OTOP1 | ENSG00000163982 | Otopetrin-1 |
| 1385 | HLA-A*30:02_ASLDSITHRY | 1171 | CD045 | ENSG00000164123 | Uncharacterized protein C4orf45 |
| 1386 | HLA-A*30:02_ASNRSLPAKY | 1172 | CD045 | ENSG00000164123 | Uncharacterized protein C4orf45 |
| 1387 | HLA-B*35:01_TPDPPTIISY | 1173 | ZPLD1 | ENSG00000170044 | Zona pellucida-like domain-containing protein 1 |
| | | | | | (ZP domain-containing protein 1) |
| 1388 | HLA-A*03:01_ALYPALPKSGK | 1174 | GDPD4 | ENSG00000178795 | Glycerophosphodiester phosphodiesterase |
| | | | | | domain-containing protein 4 (EC 3.1.-.-) |
| | | | | | (Glycerophosphodiester phosphodiesterase 6) |
| | | | | | (UgPQ) |
| 1389 | HLA-B*44:03_LETDIHLSY | 642 | GDPD4 | ENSG00000178795 | Glycerophosphodiester phosphodiesterase |
| | | | | | domain-containing protein 4 (EC 3.1.-.-) |
| | | | | | (Glycerophosphodiester phosphodiesterase 6) |
| | | | | | (UgPQ) |
| 1390 | HLA-B*18:01_SEAGLTANQY | 472 | NAL11 | ENSG00000179873 | NACHT, LRR and PYD domains-containing |
| | | | | | protein 11 (Nucleotide-binding oligomerization |
| | | | | | domain protein 17)(PAAD-and NACHT domain- |
| | | | | | containing protein 10)(PYRIN-containing |
| | | | | | APAF1-like protein 6) |
| 1391 | HLA-A*33:01_DVFSPSHKTTR | 1175 | CL040 | ENSG00000180116 | Uncharacterized protein C12orf40 |
| 1392 | HLA-A*33:01_DYYPSSSER | 1176 | CL040 | ENSG00000180116 | Uncharacterized protein C12orf40 |
| 1393 | HLA-A*02:01_ILMEEGGIYSL | 1177 | CL040 | ENSG00000180116 | Uncharacterized protein C12orf40 |
| 1394 | HLA-A*01:01_STDEIRQSDY | 1178 | CL040 | ENSG00000180116 | Uncharacterized protein C12orf40 |
| 1395 | HLA-B*08:01_TLFERLNSL | 1179 | CL040 | ENSG00000180116 | Uncharacterized protein C12orf40 |
| 1396 | HLA-A*24:02_NYSPVTGKF | 1180 | OTOL1 | ENSG00000182447 | Otolin-1 |
| 1397 | HLA-A*02:07_TLDPADFFL | 1181 | OTOL1 | ENSG00000182447 | Otolin-1 |
| 1398 | HLA-B*44:03_AEIAINVHL | 1182 | CNGA2 | ENSG00000183862 | Cyclic nucleotide-gated olfactory channel (Cyclic |
| | | | | | nucleotide-gated cation channel 2)(Cyclic |
| | | | | | nucleotide-gated channel alpha-2)(CNG channel |
| | | | | | alpha-2)(CNG-2)(CNG2) |
| 1399 | HLA-C*02:02_AEIAINVHL | 1182 | CNGA2 | ENSG00000183862 | Cyclic nucleotide-gated olfactoty channel (Cyclic |
| | | | | | nucleotide-gated cation channel 2)(Cyclic |
| | | | | | nucleotide-gated channel alpha-2)(CNG channel |
| | | | | | alpha-2)(CNG-2)(CNG2) |
| 1400 | HLA-C*02:02_AEYTGAQQKL | 1183 | CNGA2 | ENSG00000183862 | Cyclic nucleotide-gated olfactoty channel (Cyclic |
| | | | | | nucleotide-gated cation channel 2)(Cyclic |
| | | | | | nucleotide-gated channel alpha-2)(CNG channel |
| | | | | | alpha-2)(CNG-2)(CNG2) |
| 1401 | HLA-A*03:01_AINVHLSTLK | 1184 | CNGA2 | ENSG00000183862 | Cyclic nucleotide-gated olfactoty channel (Cyclic |
| | | | | | nucleotide-gated cation channel 2)(Cyclic |
| | | | | | nucleotide-gated channel alpha-2)(CNG channel |
| | | | | | alpha-2)(CNG-2)(CNG2) |
| 1402 | HLA-B*18:01_DENEVATSM | 1185 | CNGA2 | ENSG00000183862 | Cyclic nucleotide-gated olfactoly channel (Cyclic |
| | | | | | nucleotide-gated cation channel 2)(Cyclic |
| | | | | | nucleotide-gated channel alpha-2)(CNG channel |
| | | | | | alpha-2)(CNG-2)(CNG2) |
| 1403 | HLA-A*01:01_FSDLQKGYY | 1186 | CNGA2 | ENSG00000183862 | Cyclic nucleotide-gated olfactoly channel (Cyclic |
| | | | | | nucleotide-gated cation channel 2)(Cyclic |
| | | | | | nucleotide-gated channel alpha-2)(CNG channel |
| | | | | | alpha-2)(CNG-2)(CNG2) |
| 1404 | HLA-B*44:03_MEVDVQEKL | 1187 | CNGA2 | ENSG00000183862 | Cyclic nucleotide-gated olfactoly channel (Cyclic |
| | | | | | nucleotide-gated cation channel 2)(Cyclic |
| | | | | | nucleotide-gated channel alpha-2)(CNG channel |
| | | | | | alpha-2)(CNG-2)(CNG2) |
| 1405 | HLA-A*01:01_VADDGVTQY | 1188 | CNGA2 | ENSG00000183862 | Cyclic nucleotide-gated olfactoty channel (Cyclic |
| | | | | | nucleotide-gated cation channel 2)(Cyclic |
| | | | | | nucleotide-gated channel alpha-2)(CNG channel |
| | | | | | alpha-2)(CNG-2)(CNG2) |
| 1406 | HLA-A*01:01_VLDPAGDWYY | 1189 | CNGA2 | ENSG00000183862 | Cyclic nucleotide-gated olfactoty channel (Cyclic |
| | | | | | nucleotide-gated cation channel 2)(Cyclic |
| | | | | | nucleotide-gated channel alpha-2)(CNG channel |
| | | | | | alpha-2)(CNG-2)(CNG2) |
| 1407 | HLA-B*35:01_YPNITDPEY | 1190 | CNGA2 | ENSG00000183862 | Cyclic nucleotide-gated olfactoty channel (Cyclic |
| | | | | | nucleotide-gated cation channel 2)(Cyclic |
| | | | | | nucleotide-gated channel alpha-2)(CNG channel |
| | | | | | alpha-2)(CNG-2)(CNG2) |
| 1408 | HLA-A*29:02_AFLTSTLLF | 1191 | PLET1 | ENSG00000188771 | Placenta-expressed transcript 1 protein |
| 1409 | HLA-B*18:01_DEYYTITL | 1192 | PLET1 | ENSG00000188771 | Placenta-expressed transcript 1 protein |
| 1410 | HLA-B*18:01_TEVEIQAF | 1193 | PLET1 | ENSG00000188771 | Placenta-expressed transcript 1 protein |
| 1411 | HLA-B*18:01_YENVAKVGF | 1194 | ZN560 | ENSG00000198028 | Zinc finger protein 560 |
| 1412 | HLA-C*02:02_AENQGLVLKF | 344 | A0A1B0GTN1 | ENSG00000224960 | Putative SMEK homolog 3 |
| 1413 | HLA-A*02:07_LLDDIMAEV | 711 | F2Z2I4 | ENSG00000228927, | Testis-specific Y-encoded protein 10 (Testis- |
| | | | | ENSG00000236424 | specific Y-encoded protein 3) |
| 1414 | HLA-A*02:07_LLDDIMAEV | 711 | TSPY3 | ENSG00000228927 | Testis-specific Y-encoded protein 3 |
| 1415 | HLA-A*01:01_ATEVSTWFY | 1195 | AT5L2 | ENSG00000249222 | ATP synthase subunit g 2, mitochondrial (ATPase |
| | | | | | subunit g 2) |
| 1416 | HLA-A*02:01_NLVEKTPAL | 1196 | AT5L2 | ENSG00000249222 | ATP synthase subunit g 2, mitochondrial (ATPase |
| | | | | | subunit g 2) |
| 1417 | HLA-B*35:01_TPALVNAAVTY | 1197 | AT5L2 | ENSG00000249222 | ATP synthase subunit g 2, mitochondrial (ATPase |
| | | | | | subunit g 2) |
| 1418 | HLA-A*02:07_ALDGISQVL | 1198 | PTTG2 | ENSG00000250254 | Securin-2 (Pituitary tumor-transforming gene 2 |
| | | | | | protein) |
| 1419 | HLA-B*35:01_DAYPEIEKF | 1199 | PTTG2 | ENSG00000250254 | Securin-2 (Pituitary tumor-transforming gene 2 |
| | | | | | protein) |
| 1420 | HLA-B*51:01_DAYPEIEKF | 1199 | PTTG2 | ENSG00000250254 | Securin-2 (Pituitary tumor-transforming gene 2 |
| | | | | | protein) |
| 1421 | HLA-B*44:03_EEGELEKLF | 1200 | PTTG2 | ENSG00000250254 | Securin-2 (Pituitary tumor-transforming gene 2 |
| | | | | | protein) |
| 1422 | HLA-A*03:01_KTYDAPSALPK | 1201 | PTTG2 | ENSG00000250254 | Securin-2 (Pituitary tumor-transforming gene 2 |
| | | | | | protein) |
| 1423 | HLA-A*11:01_KTYDAPSALPK | 1201 | PTTG2 | ENSG00000250254 | Securin-2 (Pituitary tumor-transforming gene 2 |
| | | | | | protein) |
| 1424 | HLA-A*29:02_SVYVGDALLY | 1202 | TRPC5 | ENSG00000072315 | Short transient receptor potential channel 5 |
| | | | | | (TipC5)(Transient receptor protein 5)(TRP-5) |
| | | | | | (hTRP-5)(hTRP5) |
| 1425 | HLA-A*02:07_EMFPKFTEV | 1203 | BRDT | ENSG00000137948 | Brodomain testis-specific protein |
| | | | | | (Cancer/testis antigen 9)(CT9)(RING3-like |
| | | | | | protein) |
| 1426 | HLA-A*11:01_AVVDGIQYK | 1204 | ADAD1 | ENSG00000164113 | Adenosine deaminase domain-containing protein |
| | | | | | 1 (Testis nuclear RNA-binding protein) |
| 1427 | HLA-A*33:01_DTHAVVTAR | 1205 | ADAD1 | ENSG00000164113 | Adenosine deaminase domain-containing protein |
| | | | | | 1 (Testis nuclear RNA-binding protein) |
| 1428 | HLA-A*26:01_EVVAIGTGEY | 1206 | ADAD1 | ENSG00000164113 | Adenosine deaminase domain-containing protein |
| | | | | | 1 (Testis nuclear RNA-binding protein) |
| 1429 | HLA-A*11:01_ISNPVLPPK | 1207 | ADAD1 | ENSG00000164113 | Adenosine deaminase domain-containing protein |
| | | | | | 1 (Testis nuclear RNA-binding protein) |
| 1430 | HLA-A*11:01_SISNPVLPPK | 1208 | ADAD1 | ENSG00000164113 | Adenosine deaminase domain-containing protein |
| | | | | | 1 (Testis nuclear RNA-binding protein) |
| 1431 | HLA-A*31:01_SLAAFIIER | 1209 | ADAD1 | ENSG00000164113 | Adenosine deaminase domain-containing protein |
| | | | | | 1 (Testis nuclear RNA-binding protein) |
| 1432 | HLA-B*35:01_HAGPNVYKF | 1210 | MAJIN | ENSG00000168070 | Membrane-anchored junction protein |
| 1433 | HLA-B*44:02_AESDVTRFLF | 1211 | HYPM | ENSG00000187516 | Huntingtin-interacting protein M (Huntinglin |
| | | | | | yeast partner M) |
| 1434 | HLA-B*44:03_AESDVTRFLF | 1211 | HYPM | ENSG00000187516 | Huntingtin-interacting protein M (Huntinglin |
| | | | | | yeast partner M) |
| 1435 | HLA-B*44:02_AESDVTRFL | 1212 | HYPM | ENSG00000187516 | Huntingtin-interacting protein M (Huntinglin |
| | | | | | yeast partner M) |
| 1436 | HLA-B*44:03_AESDVTRFL | 1212 | HYPM | ENSG00000187516 | Huntingtin-interacting protein M (Huntinglin |
| | | | | | yeast partner M) |
| 1437 | HLA-A*31:01_QVKTWFQNR | 1213 | BSH | ENSG00000188909 | Brain-specific homeobox protein homolog |
| 1438 | HLA-B*51:01_YPLMPTIL | 1214 | BSH | ENSG00000188909 | Brain-specific homeobox protein homolog |
| 1439 | HLA-B*18:01_EEETLKTLY | 1215 | Q5VXJ5 | ENSG00000198765 | Synaptonemal complex protein 1 (Fragment) |
| 1440 | HLA-B*44:03_EEETLKTLY | 1215 | Q5VXJ5 | ENSG00000198765 | Synaptonemal complex protein 1 (Fragment) |
| 1441 | HLA-C*05:01_IADEAVKL | 1216 | Q5VXJ5 | ENSG00000198765 | Synaptonemal complex protein 1 (Fragment) |
| 1442 | HLA-A*30:02_KQKFGEITDTY | 1217 | Q5VXJ5 | ENSG00000198765 | Synaptonemal complex protein 1 (Fragment) |
| 1443 | HLA-A*29:02_KVLGEKETLLY | 1218 | Q5VXJ5 | ENSG00000198765 | Synaptonemal complex protein 1 (Fragment) |
| 1444 | HLA-B*44:02_SEEETLKTLY | 1219 | Q5VXJ5 | ENSG00000198765 | Synaptonemal complex protein 1 (Fragment) |
| 1445 | HLA-B*44:03_SEEETLKTLY | 1219 | Q5VXJ5 | ENSG00000198765 | Synaptonemal complex protein 1 (Fragment) |
| 1446 | HLA-C*02:02_SEEETLKTLY | 1219 | Q5VXJ5 | ENSG00000198765 | Synaptonemal complex protein 1 (Fragment) |
| 1447 | HLA-B*35:01_TAITTSEQY | 1220 | Q5VXJ5 | ENSG00000198765 | Synaptonemal complex protein 1 (Fragment) |
| 1448 | HLA-A*11:01_TSVDHGISK | 1221 | Q5VXJ5 | ENSG00000198765 | Synaptonemal complex protein 1 (Fragment) |
| 1449 | HLA-A*11:01_TTSEQYYSK | 1222 | Q5VXJ5 | ENSG00000198765 | Synaptonemal complex protein 1 (Fragment) |
| 1450 | HLA-A*01:01_VSEEETLKTLY | 1223 | Q5VXJ5 | ENSG00000198765 | Synaptonemal complex protein 1 (Fragment) |
| 1451 | HLA-B*51:01_DAPKEINI | 1224 | RGS21 | ENSG00000253148 | Regulator of G-protein signaling 21 (RGS21) |
| 1452 | HLA-A*02:01_GLDAFRIFL | 1225 | RGS21 | ENSG00000253148 | Regulator of G-protein signaling 21 (RGS21) |
| 1453 | HLA-A*02:07_GLDAFRIFL | 1225 | RGS21 | ENSG00000253148 | Regulator of G-protein signaling 21 (RGS21) |
| 1454 | HLA-B*51:01_LPIEGQEI | 1226 | BUD | ENSG00000259571 | BH3-like motif-containing cell death inducer |
| | | | | | (Breast cancer cell protein 2) |
| 1455 | HLA-A*24:02_LYIGATGQF | 1227 | TCF24 | ENSG00000261787 | Transcription factor 24 (TCF-24) |
| 1456 | HLA-C*05:01_FTDEGDQLF | 1228 | SE1L2 | ENSG00000101251 | Protein sel-1 homolog 2 (Suppressor of lin-12- |
| | | | | | like protein 2)(Sel-1L2) |
| 1457 | HLA-A*29:02_GLHGLGLLY | 1229 | SE1L2 | ENSG00000101251 | Protein sel-1 homolog 2 (Suppressor of lin-12- |
| | | | | | like protein 2)(Sel-1L2) |
| 1458 | HLA-A*24:02_LYIKSLPTF | 1230 | SE1L2 | ENSG00000101251 | Protein sel-1 homolog 2 (Suppressor of lin-12- |
| | | | | | like protein 2)(Sel-1L2) |
| 1459 | HLA-B*35:01_NALGFLSSY | 1231 | SE1L2 | ENSG00000101251 | Protein sel-1 homolog 2 (Suppressor of lin-12- |
| | | | | | like protein 2)(Sel-1L2) |
| 1460 | HLA-B*44:03_TEIVLENNY | 305 | R4GMQ3 | ENSG00000107831 | Fibroblast growth factor 8 |
| 1461 | HLA-B*18:01_TEDIGPQF | 1232 | NMUR2 | ENSG00000132911 | Neuromedin-U receptor 2 (NMU-R2)(G-protein |
| | | | | | coupled receptor FM-4)(G-protein coupled |
| | | | | | receptor TGR-1) |
| 1462 | HLA-A*02:07_FLQEVITTV | 732 | ZAN | ENSG00000146839 | Zonadhesin |
| 1463 | HLA-A*01:01_HGDAHLQEY | 1233 | FATE1 | ENSG00000147378 | Fetal and adult testis-expressed transcript protein |
| | | | | | (Cancer/testis antigen 43)(CT43)(Tumor antigen |
| | | | | | BJ-HCC-2) |
| 1464 | HLA-A*11:01_STKPDMIQK | 1234 | IFNK | ENSG00000147896 | Interferon kappa (IFN-kappa) |
| 1465 | HLA-B*35:01_QPLQPSSPVAY | 1235 | PASD1 | ENSG00000166049 | Circadian clock protein PASD1 (Cancer/testis |
| | | | | | antigen 63)(CT63)(OX-TES-1)(PAS domain- |
| | | | | | containing protein 1) |
| 1466 | HLA-A*02:01_SLGPVVQV | 1236 | PASD1 | ENSG00000166049 | Circadian clock protein PASD1 (Cancer/testis |
| | | | | | antigen 63)(CT63)(OX-TES-1)(PAS domain- |
| | | | | | containing protein 1) |
| 1467 | HLA-A*30:02_AVSISTVGY | 1237 | KCNV2 | ENSG00000168263 | Potassium voltage-gated channel subfamily V |
| | | | | | member 2 (Voltage-gated potassium channel |
| | | | | | subunit Kv8.2) |
| 1468 | HLA-B*44:03_EEEQLQQARW | 1238 | CC185 | ENSG00000178395 | Coiled-coil domain-containing protein 185 |
| 1469 | HLA-B*44:02_EEQLQQARW | 1239 | CC185 | ENSG00000178395 | Coiled-coil domain-containing protein 185 |
| 1470 | HLA-B*44:03_EEQLQQARW | 1239 | CC185 | ENSG00000178395 | Coiled-coil domain-containing protein 185 |
| 1471 | HLA-B*35:01_FPVQATIDFY | 1240 | DSCR6 | ENSG00000183145 | Protein ripply3 (Down syndrome critical region |
| | | | | | protein 6) |
| 1472 | HLA-C*02:02_KEGEPVEFTF | 241 | LN28B | ENSG00000187772 | Protein lin-28 homolog B (Lin-28B) |
| 1473 | HLA-B*18:01_LEQLVLMY | 1241 | CS067 | ENSG00000188032 | UPF0575 protein C19orf67 |
| 1474 | HLA-A*30:02_RVAELINASY | 1085 | DC8L2 | ENSG00000189186 | DDB1-and CUL4-associated factor 8-like protein |
| | | | | | 2 (WD repeat-containing protein 42C) |
| 1475 | HLA-A*26:01_HVAGEQMAEY | 1242 | BTNL2 | ENSG00000204290 | Butyrophilin-like protein 2 (BTL-II) |
| 1476 | HLA-B*18:01_TEMQMEEY | 1243 | BTNL2 | ENSG00000204290 | Butyrophilin-like protein 2 (BTL-II) |
| 1477 | HLA-A*01:01_VTEMQMEEY | 1244 | BTNL2 | ENSG00000204290 | Butyrophilin-like protein 2 (BTL-II) |
| 1478 | HLA-B*18:01_MEFKAVIY | 1245 | GNAT3 | ENSG00000214415 | Guanine nucleotide-binding protein G(t) subunit |
| | | | | | alpha-3 (Gustducin alpha-3 chain) |
| 1479 | HLA-A*24:02_YYLNDLDRI | 1246 | GNAT3 | ENSG00000214415 | Guanine nucleotide-binding protein G(t) subunit |
| | | | | | alpha-3 (Gustducin alpha-3 chain) |
| 1480 | HLA-A*24:02_RYILENHDF | 1247 | RFPLB | ENSG00000251258 | Ret finger protein-like 4B (RING finger protein |
| | | | | | 211) |
| 1481 | HLA-A*30:02_TLQEGITGVY | 1248 | S35G6 | ENSG00000259224 | Solute carrier family 35 member G6 (Acyl- |
| | | | | | malonyl-condensing enzyme 1-like protein 3) |
| | | | | | (Transmembrane protein 21B) |
| 1482 | HLA-A*02:01_TLQEGITGV | 1249 | S35G6 | ENSG00000259224 | Solute carrier family 35 member G6 (Acyl- |
| | | | | | malonyl-condensing enzyme 1-like protein 3) |
| | | | | | (Transmembrane protein 21B) |
| 1483 | HLA-A*33:01_EYGNIPVVR | 1250 | TRPC7 | ENSG00000069018 | Short transient receptor potential channel 7 |
| | | | | | (TIpC7)(Transient receptor protein 7)(TRP-7) |
| | | | | | (hTRP7) |
| 1484 | HLA-A*03:01_GIADPNQSAK | 1251 | ADAM7 | ENSG00000069206 | Disintegrin and metalloproteinase domain- |
| | | | | | containing protein 7 (ADAM 7)(Sperm |
| | | | | | maturation-related glycoprotein GP-83) |
| 1485 | HLA-A*11:01_ATLELNETQVK | 1252 | HXB1 | ENSG00000120094 | Homeobox protein Hox-Bl (Homeobox protein |
| | | | | | Hox-2I) |
| 1486 | HLA-B*46:01_SINKSGASY | 1253 | TTLL2 | ENSG00000120440 | Probable tubulin polyglutamylase TTLL2 (EC 6.- |
| | | | | | .-.-)(Testis-specific protein NYD-TSPG) |
| | | | | | (Tubulin--tyro ine ligase-like protein 2) |
| 1487 | HLA-B*18:01_EEEKLFLSY | 680 | APOL5 | ENSG00000128313 | Apolipoprotein L5 (Apolipoprotein L-V)(ApoL- |
| | | | | | V) |
| 1488 | HLA-B*35:01_FPIVGDVAL | 1254 | TSN16 | ENSG00000130167 | Tetraspanin-16 (Tspan-16)(Tetraspanin TM4-B) |
| | | | | | (Transmembrane 4 superfamily member 16) |
| 1489 | HLA-A*02:07_MLDDIPEDNTL | 1255 | SPT22 | ENSG00000141255 | Spermatogenesis-associated protein 22 (Testis |
| | | | | | development protein NYD-SP20) |
| 1490 | HLA-C*16:01_AAFVSSRVL | 1256 | X6R6V8 | ENSG00000143552 | Nuclear pore membrane glycoprotein 210-like |
| 1491 | HLA-A*11:01_ATGSANMTK | 1257 | SPI2A | ENSG00000147059 | Spindlin-2A (Protein DXF34)(Spindlin-like |
| | | | | | protein 2A)(SPIN-2)(SPIN-2A) |
| 1492 | HLA-A*30:02_GVQTFTSGKY | 1258 | TRI49 | ENSG00000168930 | Tripartite motif-containing protein 49 (RING |
| | | | | | finger protein 18)(Testis-specific RING-finger |
| | | | | | protein) |
| 1493 | HLA-B*35:01_MPQPLNPEL | 1259 | TRI49 | ENSG00000168930 | Tripartite motif-containing protein 49 (RING |
| | | | | | finger protein 18)(Testis-specific RING-finger |
| | | | | | protein) |
| 1494 | HLA-A*24:02_NYFIDPVTI | 554 | TRI49 | ENSG00000168930 | Tripartite motif-containing protein 49 (RING |
| | | | | | finger protein 18)(Testis-specific RING-finger |
| | | | | | protein) |
| 1495 | HLA-A*11:01_SSQPSPSDPK | 1260 | VCX3 | ENSG00000169059 | Variable charge X-linked protein 3 (Variable |
| | | | | | charge protein on X with eight repeats)(VCX-8r) |
| | | | | | (Variably charged protein X-A)(VCX-A) |
| 1496 | HLA-B*44:03_AEYTGAQQKL | 1183 | CNGA2 | ENSG00000183862 | Cyclic nucleotide-gated olfactory channel (Cyclic |
| | | | | | nucleotide-gated cation channel 2)(Cyclic |
| | | | | | nucleotide-gated channel alpha-2)(CNG channel |
| | | | | | alpha-2)(CNG-2)(CNG2) |
| 1497 | HLA-A*29:02_ILLDPVQRNLY | 1261 | ZN560 | ENSG00000198028 | Zinc finger protein 560 |
| 1498 | HLA-A*11:01_AGESGKSTIVK | 1262 | GNAT3 | ENSG00000214415 | Guanine nucleotide-binding protein G(t) subunit |
| | | | | | alpha-3 (Gustducin alpha-3 chain) |
| 1499 | HLA-A*26:01_EIIIYGPAY | 1263 | PSG1 | ENSG00000231924 | Pregnancy-specific beta-1-glycoprotein 1 (PS- |
| | | | | | beta-G-1)(PSBG-1)(Pregnancy-specific |
| | | | | | glycoprotein 1)(CD66 antigen-like family |
| | | | | | member F)(Fetal liver non-specific cross-reactive |
| | | | | | antigen 1/2)(FL-NCA-1/2)(PSG95)(Pregnancy- |
| | | | | | specific beta-1 glycoprotein CID)(PS-beta-C/D) |
| | | | | | (CD antigen CD66f) |
| 1500 | HLA-A*24:02_EYLTQAAFF | 1264 | BHMG1 | ENSG00000237452 | Basic helix-loop-helix and HMG box domain- |
| | | | | | containing protein 1 |
| 1501 | HLA-A*02:07_SIDQIYKKL | 1265 | SMC1B | ENSG00000077935 | Structural maintenance of chromosomes protein |
| | | | | | 1B (SMC protein 1B)(SMC-1-beta)(SMC-1B) |
| 1502 | HLA-A*11:01_IVVDKSDLIPK | 1266 | TDRD1 | ENSG00000095627 | Tudor domain-containing protein 1 (Cancer/testis |
| | | | | | antigen 41.1)(CT41.1) |
| 1503 | HLA-A*24:02_NYPETLKFMLI | 1267 | S14L3 | ENSG00000100012 | SEC14-like protein 3 (Tocopherol-associated |
| | | | | | protein 2) |
| 1504 | HLA-A*30:02_STLKFIGQY | 1268 | MORC1 | ENSG00000114487 | MORC family CW-type zinc finger protein 1 |
| | | | | | (Cancer/testis antigen 33)(CT33) |
| 1505 | HLA-B*51:01_DAPIIKEI | 1269 | H3BVE8 | ENSG00000121446 | Regulator of G-protein-signaling protein-like |
| 1506 | HLA-B*51:01_EGNPLLLTV | 1270 | H3BVE8 | ENSG00000121446 | Regulator of G-protein-signaling protein-like |
| 1507 | HLA-A*02:01_FLEGNPLLLTV | 1271 | H3BVE8 | ENSG00000121446 | Regulator of G-protein-signaling protein-like |
| 1508 | HLA-A*29:02_GFWSDTILY | 1272 | H3BVE8 | ENSG00000121446 | Regulator of G-protein-signaling protein-like |
| 1509 | HLA-C*02:02_QEMQNSKENF | 1273 | H3BVE8 | ENSG00000121446 | Regulator of G-protein-signaling protein-like |
| 1510 | HLA-A*24:02_SYLTGSAGEEL | 1274 | H3BVE8 | ENSG00000121446 | Regulator of G-protein-signaling protein-like |
| 1511 | HLA-A*24:02_VFADFFNTF | 1275 | H3BVE8 | ENSG00000121446 | Regulator of G-protein-signaling protein-like |
| 1512 | HLA-C*02:02_SEQMSRTNY | 1276 | NMUR2 | ENSG00000132911 | Neuromedin-U receptor 2 (NMU-R2)(G-protein |
| | | | | | coupled receptor FM-4)(G-protein coupled |
| | | | | | receptor TGR-1) |
| 1513 | HLA-B*44:03_MEFSGENRGY | 1277 | RBM46 | ENSG00000151962 | Probable RNA-binding protein 46 (Cancer/testis |
| | | | | | antigen 68)(CT68)(RNA-binding motif protein |
| | | | | | 46) |
| 1514 | HLA-B*08:01_HPQLRKVTL | 1278 | S22AD | ENSG00000172940 | Solute carrier family 22 member 13 (Organic |
| | | | | | cation transporter-like 3)(ORCTL-3) |
| 1515 | HLA-B*44:03_AEAPLSQRW | 1279 | CI131 | ENSG00000174038 | Uncharacterized protein C9orf131 |
| 1516 | HLA-B*18:01_VEAPVSTF | 1280 | CI131 | ENSG00000174038 | Uncharacterized protein C9orf131 |
| 1517 | HLA-A*01:01_VSEPIADQSNY | 1281 | CI131 | ENSG00000174038 | Uncharacterized protein C9orf131 |
| 1518 | HLA-A*02:07_YLDSFADGL | 1282 | TEX36 | ENSG00000175018 | Testis-expressed protein 36 |
| 1519 | HLA-A*02:01_LLFDKEPINV | 1283 | HORM2 | ENSG00000176635 | HORMA domain-containing protein 2 |
| 1520 | HLA-A*29:02_YFSHHILAVY | 1284 | FBX39 | ENSG00000177294 | F-box only protein 39 |
| 1521 | HLA-B*18:01_SEYQLNDSAAY | 1285 | GNAT3 | ENSG00000214415 | Guanine nucleotide-binding protein G(t) subunit |
| | | | | | alpha-3 (Gustducin alpha-3 chain) |
| 1522 | HLA-B*18:01_DDVIISSGY | 1286 | ACSM4 | ENSG00000215009 | Acyl-coenzyme A synthetase ACSM4, |
| | | | | | mitochondrial (EC 6.2.1.2)(Acyl-CoA synthetase |
| | | | | | medium-chain family member 4) |
| 1523 | HLA-A*03:01_AILLQVIAK | 1133 | TDR15 | ENSG00000218819 | Tudor domain-containing protein 15 |
| 1524 | HLA-B*46:01_LVASGLATY | 1287 | TDR15 | ENSG00000218819 | Tudor domain-containing protein 15 |
| 1525 | HLA-B*44:03_SENIDVISL | 1288 | TDR15 | ENSG00000218819 | Tudor domain-containing protein 15 |
| 1526 | HLA-A*29:02_TFPSLFSLY | 1289 | TDR15 | ENSG00000218819 | Tudor domain-containing protein 15 |
| 1527 | HLA-B*18:01_EEITLRENF | 1290 | RD21L | ENSG00000244588 | Double-strand-break repair protein rad21-like |
| | | | | | protein 1 |
| 1528 | HLA-B*44:03_EEITLRENF | 1290 | RD21L | ENSG00000244588 | Double-strand-break repair protein rad21-like |
| | | | | | protein 1 |
| 1529 | HLA-B*44:02_EENIETERW | 1291 | RD21L | ENSG00000244588 | Double-strand-break repair protein rad21-like |
| | | | | | protein 1 |
| 1530 | HLA-B*44:03_EENIETERW | 1291 | RD21L | ENSG00000244588 | Double-strand-break repair protein rad21-like |
| | | | | | protein 1 |
| 1531 | HLA-B*35:01_NAIDVSEHF | 1292 | RD21L | ENSG00000244588 | Double-strand-break repair protein rad21-like |
| | | | | | protein 1 |
| 1532 | HLA-B*35:01_NAITLPEEF | 1293 | RD21L | ENSG00000244588 | Double-strand-break repair protein rad21-like |
| | | | | | protein 1 |
| 1533 | HLA-A*24:02_PYADIIATM | 1294 | RD21L | ENSG00000244588 | Double-strand-break repair protein rad21-like |
| | | | | | protein 1 |
| 1534 | HLA-B*18:01_DEKLTVTSL | 1295 | ESX1 | ENSG00000123576 | Homeobox protein ESX1 (Extraembryonic, |
| | | | | | spermatogenesis, homeobox 1)[Cleaved into: |
| | | | | | Homeobox protein ESX1-N; Homeobox protein |
| | | | | | ESX1-C] |
| 1535 | HLA-B*44:02_EEAANSGYSW | 381 | PRDM7 | ENSG00000126856 | Probable histone-lysine N-methyltransferase |
| | | | | | PRDM7 (EC 2.1.1.43)(PR domain zinc finger |
| | | | | | protein 7)(PR domain-containing protein 7) |
| 1536 | HLA-B*35:01_LPDKVFIKY | 1296 | RXFP2 | ENSG00000133105 | Relaxin receptor 2 (G-protein coupled receptor |
| | | | | | 106)(G-protein coupled receptor affecting |
| | | | | | testicular descent)(Leucine-rich repeat- |
| | | | | | containing G-protein coupled receptor 8)(Relaxin |
| | | | | | family peptide receptor 2) |
| 1537 | HLA-A*02:01_AVADTLIGV | 1297 | GP119 | ENSG00000147262 | Glucose-dependent insulinotmpic receptor (G- |
| | | | | | protein coupled receptor 119) |
| 1538 | HLA-A*11:01_ASLDSITHR | 1298 | CD045 | ENSG00000164123 | Uncharacterized protein C4orf45 |
| 1539 | HLA-A*29:02_YLPGLLYKF | 1299 | ZPLD1 | ENSG00000170044 | Zona pellucida-like domain-containing protein 1 |
| | | | | | (ZP domain-containing protein 1) |
| 1540 | HLA-A*30:02_TSLDMTHPY | 1300 | SP7 | ENSG00000170374 | Transcription factor Sp7 (Zinc finger protein |
| | | | | | osterix) |
| 1541 | HLA-A*02:07_ALMEVTVYL | 470 | NAL11 | ENSG00000179873 | NACHT, LRR and PYD domains-containing |
| | | | | | protein 11 (Nucleotide-binding oligomerization |
| | | | | | domain protein 17)(PAAD-and NACHT domain- |
| | | | | | containing protein 10)(PYRIN-containing |
| | | | | | APAF1-like protein 6) |
| 1542 | HLA-B*35:01_MPQPLNPEL | 1259 | TR49B | ENSG00000182053 | Putative tripartite motif-containing protein 49B |
| | | | | | (RING finger protein 18B) |
| 1543 | HLA-B*18:01_DEHTGIHTM | 1301 | FAM9A | ENSG00000183304 | Protein FAM9A |
| 1544 | HLA-A*02:07_MLDALLVHI | 1302 | TMM89 | ENSG00000183396 | Transmembrane protein 89 |
| 1545 | HLA-C*05:01_VADDGVTQY | 1188 | CNGA2 | ENSG00000183862 | Cyclic nucleotide-gated olfactory channel (Cyclic |
| | | | | | nucleotide-gated cation chamiel 2)(Cyclic |
| | | | | | nucleotide-gated channel alpha-2)(CNG channel |
| | | | | | alpha-2)(CNG-2)(CNG2) |
| 1546 | HLA-A*11:01_ASSAPTAEK | 1303 | HOYFA1 | ENSG00000185958 | Protein FAM186A (Fragment) |
| 1547 | HLA-B*18:01_EETQILRDTF | 1304 | HOYFA1 | ENSG00000185958 | Protein FAM186A (Fragment) |
| 1548 | HLA-B*44:03_EETQILRDTF | 1304 | HOYFA1 | ENSG00000185958 | Protein FAM186A (Fragment) |
| 1549 | HLA-A*03:01_KVAQVPFTTK | 1305 | HOYFA1 | ENSG00000185958 | Protein FAM186A (Fragment) |
| 1550 | HLA-A*11:01_STSSYPIAEK | 1306 | HOYFA1 | ENSG00000185958 | Protein FAM186A (Fragment) |
| 1551 | HLA-B*46:01_VLKDVQRSY | 1307 | HOYFA1 | ENSG00000185958 | Protein FAM186A (Fragment) |
| 1552 | HLA-A*01:01_VSEAKPSQY | 1308 | HOYFA1 | ENSG00000185958 | Protein FAM186A (Fragment) |
| 1553 | HLA-A*02:07_LLPSWVPEV | 1309 | SPT21 | ENSG00000187144 | Spermatogenesis-associated protein 21 |
| 1554 | HLA-C*02:02_AESDVTRFL | 1212 | HYPM | ENSG00000187516 | Huntingtin-interacting protein M (Huntingtin |
| | | | | | yeast partner M) |
| 1555 | HLA-A*11:01_STVFDPVFK | 1310 | RFA4 | ENSG00000204086 | Replication protein A 30 kDa subunit (RP-A p30) |
| | | | | | (Replication factor A protein 4)(RF-A protein 4) |
| 1556 | HLA-A*29:02_IFSAINPVLYY | 1311 | M4A18 | ENSG00000214782 | Membrane-spanning 4-domains subfamily A |
| | | | | | member 18 |
| 1557 | HLA-A*11:01_VTTYPISPK | 1312 | M4A18 | ENSG00000214782 | Membrane-spanning 4-domains subfamily A |
| | | | | | member 18 |
| 1558 | HLA-A*29:02_LFLFGVTKY | 1313 | PSG3 | ENSG00000221826 | Pregnancy-specific beta-1-glycoprotein 3 (PS- |
| | | | | | beta-G-3)(PSBG-3)(Pregnancy-specific |
| | | | | | glycoprotein 3)(Carcinoembryonic antigen SG5) |
| 1559 | HLA-B*51:01_LPKLPKPYI | 578 | PSG3 | ENSG00000221826 | Pregnancy-specific beta-1-glycoprotein 3 (PS- |
| | | | | | beta-G-3)(PSBG-3)(Pregnancy-specific |
| | | | | | glycoprotein 3)(Carcinoembryonic antigen SG5) |
| 1560 | HLA-B*51:01_LPTTAQVTI | 579 | PSG3 | ENSG00000221826 | Pregnancy-specific beta-1-glycoprotein 3 (PS- |
| | | | | | beta-G-3)(PSBG-3)(Pregnancy-specific |
| | | | | | glycoprotein 3)(Carcinoembryonic antigen SG5) |
| 1561 | HLA-C*01:02_QVPGGSQEL | 1314 | ANHX | ENSG00000227059 | Anomalous homeobox protein |
| 1562 | HLA-A*26:01_SVANSTVAY | 615 | AP2D | ENSG00000008197 | Transcription factor AP-2-delta (AP2-delta) |
| | | | | | (Activating enhancer-binding protein 2-delta) |
| | | | | | (Transcription factor AP-2-beta-like 1) |
| 1563 | HLA-A*24:02_AYAERLGVTF | 1315 | RB40L | ENSG00000102128 | Ras-related protein Rab-40A-like (Ras-like |
| | | | | | GTPase) |
| 1564 | HLA-A*30:02_GAQGVILVY | 1316 | RB40L | ENSG00000102128 | Ras-related protein Rab-40A-like (Ras-like |
| | | | | | GTPase) |
| 1565 | HLA-B*51:01_HAPGVPKI | 1317 | RB40L | ENSG00000102128 | Ras-related protein Rab-40A-like (Ras-like |
| | | | | | GTPase) |
| 1566 | HLA-C*01:02_NIIESFTEL | 1318 | RB40L | ENSG00000102128 | Ras-related protein Rab-40A-like (Ras-like |
| | | | | | GTPase) |
| 1567 | HLA-B*35:01_QAYAERLGVTF | 1319 | RB40L | ENSG00000102128 | Ras-related protein Rab-40A-like (Ras-like |
| | | | | | GTPase) |
| 1568 | HLA-B*35:01_APANIQVSF | 1320 | FNDC7 | ENSG00000143107 | Fibronectin type III domain-containing protein 7 |
| 1569 | HLA-B*51:01_DAFSMINV | 1321 | FNDC7 | ENSG00000143107 | Fibronectin type III domain-containing protein 7 |
| 1570 | HLA-A*26:01_DTKYNITVY | 1322 | FNDC7 | ENSG00000143107 | Fibronectin type III domain-containing protein 7 |
| 1571 | HLA-A*30:02_GQSPLGDIFNY | 1323 | FNDC7 | ENSG00000143107 | Fibronectin type III domain-containing protein 7 |
| 1572 | HLA-A*11:01_TVMVSPVAK | 1324 | FNDC7 | ENSG00000143107 | Fibronectin type III domain-containing protein 7 |
| 1573 | HLA-C*01:02_VSPVAKTGL | 1325 | FNDC7 | ENSG00000143107 | Fibronectin type III domain-containing protein 7 |
| 1574 | HLA-B*51:01_SAAASVLTV | 1326 | GP119 | ENSG00000147262 | Glucose-dependent insulinotmpic receptor (G- |
| | | | | | protein coupled receptor 119) |
| 1575 | HLA-A*29:02_AFIIKTIGQLY | 1327 | BOLL | ENSG00000152430 | Protein boule-like |
| 1576 | HLA-B*51:01_DAMTAFESI | 1328 | J3KNE0 | ENSG00000153165 | RanBP2-like and GRIP domain-containing |
| | | | | | protein 3 |
| 1577 | HLA-B*44:03_EESSINYTF | 1329 | J3KNE0 | ENSG00000153165 | RanBP2-like and GRIP domain-containing |
| | | | | | protein 3 |
| 1578 | HLA-C*04:01_HFDESTTGSNF | 1330 | J3KNE0 | ENSG00000153165 | RanBP2-like and GRIP domain-containing |
| | | | | | protein 3 |
| 1579 | HLA-A*03:01_KLFPGSPAIYK | 1331 | J3KNE0 | ENSG00000153165 | RanBP2-like and GRIP domain-containing |
| | | | | | protein 3 |
| 1580 | HLA-B*18:01_NEQVVFSH | 1332 | J3KNE0 | ENSG00000153165 | RanBP2-like and GRIP domain-containing |
| | | | | | protein 3 |
| 1581 | HLA-A*11:01_SQYGKMANK | 1333 | J3KNE0 | ENSG00000153165 | RanBP2-like and GRIP domain-containing |
| | | | | | protein 3 |
| 1582 | HLA-A*11:01_SVQGSAPSPRK | 1334 | J3KNE0 | ENSG00000153165 | RanBP2-like and GRIP domain-containing |
| | | | | | protein 3 |
| 1583 | HLA-B*51:01_TAFESIKSV | 1335 | J3KNE0 | ENSG00000153165 | RanBP2-like and GRIP domain-containing |
| | | | | | protein 3 |
| 1584 | HLA-A*11:01_VVVETFANK | 1336 | J3KNE0 | ENSG00000153165 | RanBP2-like and GRIP domain-containing |
| | | | | | protein 3 |
| 1585 | HLA-B*44:03_SEIDQGKGY | 1337 | S4R404 | ENSG00000163424 | Uncharacterized protein C3orf30 |
| 1586 | HLA-C*02:02_SEIDQGKGY | 1337 | S4R404 | ENSG00000163424 | Uncharacterized protein C3orf30 |
| 1587 | HLA-A*26:01_EVVVALILQY | 1338 | S35G3 | ENSG00000164729 | Solute carrier family 35 member G3 (Acyl- |
| | | | | | malonyl-condensing enzyme 1)(Transmembrane |
| | | | | | protein 21A) |
| 1588 | HLA-B*51:01_TAYLWIRQI | 1339 | ACHA9 | ENSG00000174343 | Neuronal acetylcholine receptor subunit alpha-9 |
| | | | | | (Nicotinic acetylcholine receptor subunit alpha-9) |
| | | | | | (NACHR alpha-9) |
| 1589 | HLA-A*01:01_SSEDLHVFY | 1340 | CTSRD | ENSG00000174898 | Cation channel sperm-associated protein subunit |
| | | | | | delta (CatSper-delta)(CatSperdelta) |
| | | | | | (Transmembrane protein 146) |
| 1590 | HLA-B*46:01_VAHLELATY | 1341 | FMR1N | ENSG00000176988 | Fragile X mental retardation 1 neighbor protein |
| | | | | | (Cancer/testis antigen 37)(CT37)(Sarcoma |
| | | | | | antigen NY-SAR-35) |
| 1591 | HLA-A*11:01_ATGWGLVSK | 1342 | PRS38 | ENSG00000185888 | Serine protease 38 (EC 3.4.21.-)(Marapsin-2) |
| 1592 | HLA-A*30:02_GSSSGLSSSY | 1343 | HORN | ENSG00000197915 | Hornerin |
| 1593 | HLA-A*29:02_NAGPLNVLY | 1344 | S4R3Z8 | ENSG00000203963 | Uncharacterized protein C1orf141 (Fragment) |
| 1594 | HLA-B*35:01_NAGPLNVLY | 1344 | S4R3Z8 | ENSG00000203963 | Uncharacterized protein C1orf141 (Fragment) |
| 1595 | HLA-A*30:02_ATSPPTPGHY | 1345 | A0A0U1RQF7 | ENSG00000263201 | HCG1775037 |
| 1596 | HLA-B*44:03_KEVDPTGHSF | 379 | MAGAA | ENSG00000124260 | Melanoma-associated antigen 10 (Cancer/testis |
| | | | | | antigen 1.10)(CT1.10)(MAGE-10 antigen) |
| 1597 | HLA-B*18:01_DEMGVVGYF | 1346 | OTOR | ENSG00000125879 | Otoraplin (Fibrocyte-derived protein)(Melanoma |
| | | | | | inhibitory activity-like protein) |
| 1598 | HLA-C*01:02_AAPLAAGAL | 1347 | OCSTP | ENSG00000149635 | Osteoclast stimulatory transmembrane protein |
| | | | | | (OC-STAMP) |
| 1599 | HLA-C*02:02_AEQLVKTGW | 1348 | OCSTP | ENSG00000149635 | Osteoclast stimulatory transmembrane protein |
| | | | | | (OC-STAMP) |
| 1600 | HLA-B*44:03_SEGEGKELW | 1349 | OCSTP | ENSG00000149635 | Osteoclast stimulatory transmembrane protein |
| | | | | | (OC-STAMP) |
| 1601 | HLA-A*30:02_RLYQTDPSGTY | 1350 | PSA7L | ENSG00000154611 | Proteasome subunit alpha type-7-like (EC |
| | | | | | 3.4.25.1) |
| 1602 | HLA-A*29:02_WVQENYLEY | 75 | MAGA8 | ENSG00000156009 | Melanoma-associated antigen 8 (Cancer/testis |
| | | | | | antigen 1.8)(CT1.8)(MAGE-8 antigen) |
| 1603 | HLA-A*33:01_ELQGPWHTR | 1351 | SUN3 | ENSG00000164744 | SUN domain-containing protein 3 (Sad1/unc-84 |
| | | | | | domain-containing protein 1) |
| 1604 | HLA-A*03:01_GIFPKIMPK | 1352 | SSX3 | ENSG00000165584 | Protein SSX3 (Cancer/testis antigen 5.3)(CT5.3) |
| 1605 | HLA-A*29:02_SVFDEKQQWKF | 1353 | ESPB1 | ENSG00000169393 | Epididymal sperm-binding protein 1 (Epididymal |
| | | | | | secretory protein 12)(hE12) |
| 1606 | HLA-A*31:01_ASGPPAPAR | 798 | NGN1 | ENSG00000181965 | Neurogenin-1 (NGN-1)(Class A basic helix-loop- |
| | | | | | helix protein 6)(bHLHa6)(Neurogenic basic- |
| | | | | | helix-loop-helix protein)(Neurogenic |
| | | | | | differentiation factor 3)(NeuroD3) |
| 1607 | HLA-C*02:02_QEGSSGMELSW | 644 | TEX19 | ENSG00000182459 | Testis-expressed protein 19 |
| 1608 | HLA-A*01:01_VSDPAKIAIHY | 1354 | KCNH7 | ENSG00000184611 | Potassium voltage-gated channel subfamily H |
| | | | | | member 7 (Ether-a-go-go-related gene potassium |
| | | | | | channel 3)(ERG-3)(Eag-related protein 3) |
| | | | | | (Ether-a-go-go-related protein 3)(hERG-3) |
| | | | | | (Voltage-gated potassium channel subunit |
| | | | | | Kv11.3) |
| 1609 | HLA-A*02:01_SLVNLQPEL | 1355 | FOXE3 | ENSG00000186790 | Forkhead box protein E3 (Forkhead-related |
| | | | | | protein FKHL12)(Forkhead-related transcription |
| | | | | | factor 8)(FREAC-8) |
| 1610 | HLA-B*35:01_SPLEVPQSF | 1356 | MAGBG | ENSG00000189023 | Melanoma-associated antigen B16 (MAGE-B16 |
| | | | | | antigen) |
| 1611 | HLA-A*11:01_ATLENLLSH | 1044 | PRAM9 | ENSG00000204501 | PRAME family member 9/15 |
| 1612 | HLA-A*02:07_TLDEYLTYL | 1357 | PRAM9 | ENSG00000204501 | PRAME family member 9/15 |
| 1613 | HLA-B*51:01_DGYYRGIVV | 1358 | MO2R2 | ENSG00000206531 | Cell surface glycoprotein CD200 receptor 2 |
| | | | | | (CD200 cell surface glycoprotein receptor-like 2) |
| | | | | | (CD200 receptor-like 2)(HuCD200R2)(CD200 |
| | | | | | cell surface glycoprotein receptor-like a) |
| | | | | | (CD200RLa)(Cell surface glycoprotein CD200 |
| | | | | | receptor 1-like)(Cell surface glycoprotein 0X2 |
| | | | | | receptor 2) |
| 1614 | HLA-B*51:01_IAPDNVHVI | 1359 | M4A18 | ENSG00000214782 | Membrane-spanning 4-domains subfamily A |
| | | | | | member 18 |
| 1615 | HLA-B*08:01_DVVAKTSL | 1360 | E5RK72 | ENSG00000215262 | Potassium channel subfamily U member 1 |
| 1616 | HLA-B*44:03_GEIIIYGPAY | 1361 | PSG1 | ENSG00000231924 | Pregnancy-specific beta-1-glycoprotein 1 (PS- |
| | | | | | beta-G-1)(PSBG-1)(Pregnancy-specific |
| | | | | | glycoprotein 1)(CD66 antigen-like family |
| | | | | | member F)(Fetal liver non-specific cross-reactive |
| | | | | | antigen 1/2)(FL-NCA-1/2)(PSG95)(Pregnancy- |
| | | | | | specific beta-1 glycoprotein C/D)(PS-beta-C/D) |
| | | | | | (CD antigen CD66f) |
| 1617 | HLA-B*35:01_HPTLGPSAF | 1362 | TIFAB | ENSG00000255833 | TRAF-interacting protein with FHA domain- |
| | | | | | containing protein B (TWA-like protein) |
| 1618 | HLA-A*33:01_DYRDKSPQNR | 1363 | ERVV2 | ENSG00000268964 | Endogenous retrovirus group V member 2 Env |
| | | | | | polyprotein (HERV-V_19q13.41 provirus |
| | | | | | ancestral Env polyprotein 2) |
| 1619 | HLA-A*02:01_SLLNGEVAV | 1364 | TSN16 | ENSG00000130167 | Tetraspanin-16 (Tspan-16)(Tetraspanin TM4-B) |
| | | | | | (Transmembrane 4 superfamily member 16) |
| 1620 | HLA-B*18:01_AEVLAQSF | 1365 | PRD12 | ENSG00000130711 | PR domain zinc finger protein 12 (EC 2.1.1.-) |
| | | | | | (PR domain-containing protein 12) |
| 1621 | HLA-A*26:01_EVFNEDGTVRY | 1366 | PRD12 | ENSG00000130711 | PR domain zinc finger protein 12 (EC 2.1.1.-) |
| | | | | | (PR domain-containing protein 12) |
| 1622 | HLA-A*26:01_EVITSDILHSF | 1367 | PRD12 | ENSG00000130711 | PR domain zinc finger protein 12 (EC 2.1.1.-) |
| | | | | | (PR domain-containing protein 12) |
| 1623 | HLA-A*26:01_EVVQIGTSIF | 1368 | PRD12 | ENSG00000130711 | PR domain zinc finger protein 12 (EC 2.1.1.-) |
| | | | | | (PR domain-containing protein 12) |
| 1624 | HLA-A*11:01_SVLPAEALVLK | 1369 | PRD12 | ENSG00000130711 | PR domain zinc finger protein 12 (EC 2.1.1.-) |
| | | | | | (PR domain-containing protein 12) |
| 1625 | HLA-A*01:01_TSDILHSFLY | 1370 | PRD12 | ENSG00000130711 | PR domain zinc finger protein 12 (EC 2.1.1.-) |
| | | | | | (PR domain-containing protein 12) |
| 1626 | HLA-A*02:07_TLADALHTL | 596 | MSGN1 | ENSG00000151379 | Mesogenin-1 (Paraxial mesoderm-specific |
| | | | | | mesogenin1)(pMesogenin1)(pMsgn1) |
| 1627 | HLA-A*11:01_RTMVFVETK | 1371 | DDX4 | ENSG00000152670 | Probable ATP-dependent RNA helicase DDX4 |
| | | | | | (EC 3.6.4.13)(DEAD box protein 4)(Vasa |
| | | | | | homolog) |
| 1628 | HLA-B*44:03_REVLPLATF | 1372 | TERT | ENSG00000164362 | Telomerase reverse transcriptase (EC 2.7.7.49) |
| | | | | | (HEST2)(Telomerase catalytic subunit) |
| | | | | | (Telomerase-associated protein 2)(TP2) |
| 1629 | HLA-A*26:01_ETTGKVIYF | 1373 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 1630 | HLA-A*03:01_LLFWKPLRY | 172 | S6A18 | ENSG00000164363 | Sodium-dependent neutral amino acid transporter |
| | | | | | B(0)AT3 (Sodium-and chloride-dependent |
| | | | | | transporter XTRP2)(Solute carrier family 6 |
| | | | | | member 18)(System B(0) neutral amino acid |
| | | | | | transporter AT3) |
| 1631 | HLA-A*26:01_DSVPLIAQY | 1374 | CRFM7 | ENSG00000166664 | CHRNA7-FAM7A fusion protein (CHRNA7- |
| | | | | | DR1)(D-10) |
| 1632 | HLA-B*44:03_AEAALQTLL | 1375 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1633 | HLA-B*44:03_AEDQGFQFSY | 1376 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1634 | HLA-B*18:01_DEFVVLAL | 1377 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1635 | HLA-A*26:01_EAISQIASF | 1378 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1636 | HLA-A*26:01_EVISMGTSV | 1379 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1637 | HLA-A*02:07_HLWDPNPKIGV | 1380 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1638 | HLA-A*02:07_HMDTVVVNL | 1381 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1639 | HLA-A*02:07_ILDDAIVQRL | 1382 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1640 | HLA-A*11:01_IVMGDLSTK | 1383 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1641 | HLA-A*01:01_LTDRDVSFY | 1384 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1642 | HLA-B*18:01_DEIFNTEAM | 1385 | SL9C1 | ENSG00000172139 | Sodium/hydrogen exchanger 10 (Na(+)/H(+) |
| | | | | | exchanger 10)(NHE-10)(Solute carrier family 9 |
| | | | | | member 10)(Solute carrier family 9 member Cl) |
| | | | | | (Sperm-specific Na(+)/H(+) exchanger)(sNHE) |
| 1643 | HLA-B*44:03_SEVEEPLTVW | 1386 | VCX2 | ENSG00000177504 | Variable charge X-linked protein 2 (Variable |
| | | | | | charge protein on X with two repeats)(VCX-2r) |
| | | | | | (Variably charged protein X-B)(VCX-B) |
| 1644 | HLA-A*02:01_LLWERIELYL | 1387 | GDPD4 | ENSG00000178795 | Glycerophosphodiester phosphodiesterase |
| | | | | | domain-containing protein 4 (EC 3.1.-.-) |
| | | | | | (Glycerophosphodiester phosphodiesterase 6) |
| | | | | | (UgpQ) |
| 1645 | HLA-A*11:01_SSQPSPSDPK | 1260 | VCX1 | ENSG00000182583 | Variable charge X-linked protein 1 (Variable |
| | | | | | charge protein on X with ten repeats)(VCX-10r) |
| | | | | | (Variably charged protein X-B1)(VCX-B1) |
| 1646 | HLA-A*33:01_EMYAIYQQR | 1388 | SAMD7 | ENSG00000187033 | Sterile alpha motif domain-containing protein 7 |
| | | | | | (SAM domain-containing protein 7) |
| 1647 | HLA-A*03:01_SSYNRGLISK | 196 | NPSR1 | ENSG00000187258 | Neuropeptide S receptor (G-protein coupled |
| | | | | | receptor 154)(G-protein coupled receptor |
| | | | | | PGR14)(G-protein coupled receptor for asthma |
| | | | | | susceptibility) |
| 1648 | HLA-A*02:01_GLLEISQQL | 1389 | CS067 | ENSG00000188032 | UPF0575 protein Cq9orf67 |
| 1649 | HLA-A*02:01_VLITAVVEV | 1390 | RN133 | ENSG00000188050 | E3 ubiquitin-protein ligase RNF133 (EC |
| | | | | | 2.3.2.27)(RING finger protein 133)(RING-type |
| | | | | | E3 ubiquitin transferase RNF133) |
| 1650 | HLA-A*02:07_FMDFLQTLL | 1391 | SG1C1 | ENSG00000188076 | Secretoglobin family 1C member 1 |
| | | | | | (Secretoglobin RYD5) |
| 1651 | HLA-B*44:03_SESSTILVVRY | 316 | SPNXD | ENSG00000196406 | Sperm protein associated with the nucleus on the |
| | | | | | X chromosome D (Cancer/testis antigen 11.4) |
| | | | | | (CT11.4)(Nuclear-associated protein SPAN-Xd) |
| | | | | | (SPANX-D)(SPANX family member D) |
| 1652 | HLA-A*26:01_EAPGLGGTY | 1392 | ONEC3 | ENSG00000205922 | One cut domain family member 3 (One cut |
| | | | | | homeobox 3)(Transcription factor ONECUT-3) |
| | | | | | (OC-3) |
| 1653 | HLA-B*35:01_MPVPGQQSM | 1393 | AMELY | ENSG00000099721 | Amelogenin, Y isofonn |
| 1654 | HLA-A*02:07_FLEGNPLLLTV | 1271 | H3BVE8 | ENSG00000121446 | Regulator of G-protein-signaling protein-like |
| 1655 | HLA-C*02:02_AESEGTKAV | 1394 | H2BWT | ENSG00000123569 | Histone H2B type W-T (H2B histone family |
| | | | | | member W testis-specific) |
| 1656 | HLA-C*16:01_AESEGTKAV | 1394 | H2BWT | ENSG00000123569 | Histone H2B type W-T (H2B histone family |
| | | | | | member W testis-specific) |
| 1657 | HLA-A*11:01_RVVPPASNMLK | 1395 | CRIS1 | ENSG00000124812 | Cysteine-rich secretory protein 1 (CRISP-1) |
| | | | | | (AEG-like protein)(ARP)(Acidic epididymal |
| | | | | | glycoprotein homolog) |
| 1658 | HLA-A*01:01_TTDDDITTDHY | 1396 | CRIS1 | ENSG00000124812 | Cysteine-rich secretory protein 1 (CRISP-1) |
| | | | | | AEG-like protein)(ARP)(Acidic epididymal |
| | | | | | glycoprotein homolog) |
| 1659 | HLA-C*01:02_VVPPASNML | 1397 | CRIS1 | ENSG00000124812 | Cysteine-rich secretory protein 1 (CRISP-1) |
| | | | | | (AEG-like protein)(ARP)(Acidic epididymal |
| | | | | | glycoprotein homolog) |
| 1660 | HLA-B*51:01_YPVSWSSVI | 1398 | CRIS1 | ENSG00000124812 | Cysteine-rich secretory protein 1 (CRISP-1) |
| | | | | | (AEG-like protein)(ARP)(Acidic epididymal |
| | | | | | glycoprotein homolog) |
| 1661 | HLA-A*03:01_STASIFLAY | 913 | X6R6V8 | ENSG00000143552 | Nuclear pore membrane glycoprotein 210-like |
| 1662 | HLA-B*44:03_AEQLVKTGW | 1348 | OCSTP | ENSG00000149635 | Osteoclast stimulatcuy transmembrane protein |
| | | | | | (OC-STAMP) |
| 1663 | HLA-A*11:01_ATQNAVKLIDK | 1399 | CCD83 | ENSG00000150676 | Coiled-coil domain-containing protein 83 |
| 1664 | HLA-A*01:01_DTDMKYLLY | 1400 | CCD83 | ENSG00000150676 | Coiled-coil domain-containing protein 83 |
| 1665 | HLA-A*33:01_EYKNVGSER | 1401 | CCD83 | ENSG00000150676 | Coiled-coil domain-containing protein 83 |
| 1666 | HLA-A*01:01_GTEFGDTDMKY | 1402 | CCD83 | ENSG00000150676 | Coiled-coil domain-containing protein 83 |
| 1667 | HLA-B*44:03_TEFGDTDMKY | 1403 | CCD83 | ENSG00000150676 | Coiled-coil domain-containing protein 83 |
| 1668 | HLA-C*02:02_KEVDPAGHSY | 1404 | MAGA8 | ENSG00000156009 | Melanoma-associated antigen 8 (Cancer/tests |
| | | | | | antigen 1.8)(CT1.8)(MAGE-8 antigen) |
| 1669 | HLA-A*29:02_IVFSEEFEY | 1405 | SL9C2 | ENSG00000162753 | Sodium/hydrogen exchanger 11 (Na(+)/H(+) |
| | | | | | exchanger 11)(NHE-11)(Solute carrier family 9 |
| | | | | | member 11)(Solute carrier family 9 member C2) |
| 1670 | HLA-B*08:01_DVKMKAVM | 1406 | PLS2 | ENSG00000163746 | Phospholipid scramblase 2 (PL scramblase 2) |
| | | | | | (Ca(2+)-dependent phospholipid scramblase 2) |
| 1671 | HLA-A*29:02_YLYPFNIEY | 1407 | OTOP1 | ENSG00000163982 | Otopetrin-1 |
| 1672 | HLA-A*11:01_SSISNPVLPPK | 1408 | ADAD1 | ENSG00000164113 | Adenosine deaminase domain-containing protein |
| | | | | | 1 (Testis nuclear RNA-binding protein) |
| 1673 | HLA-B*35:01_FPFTYKGSVY | 1409 | ESPB1 | ENSG00000169393 | Epididymal sperm-binding protein 1 (Epididymal |
| | | | | | secretory protein 12)(hE12) |
| 1674 | HLA-A*30:02_STIPGVSAY | 249 | ZPLD1 | ENSG00000170044 | Zona pellucida-like domain-containing protein 1 |
| | | | | | (ZP domain-containing protein 1) |
| 1675 | HLA-A*02:01_HLWDPNPKIGV | 1380 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1676 | HLA-B*18:01_DENGQSASY | 1410 | ATS20 | ENSG00000173157 | A disintegrin and metalloproteinase with |
| | | | | | thrombospondin motifs 20 (ADAM-TS 20) |
| | | | | | (ADAM-TS20)(ADAMTS-20)(EC 3.4.24.-) |
| 1677 | HLA-B*44:03_AEVELIDQTL | 1411 | TMPS7 | ENSG00000176040 | Transmembrane protease serine 7 (EC 3.4.21.-) |
| | | | | | (Matriptase-3) |
| 1678 | HLA-B*35:01_LPIRSSILY | 1412 | TMPS7 | ENSG00000176040 | Transmembrane protease serine 7 (EC 3.4.21.-) |
| | | | | | (Matriptase-3) |
| 1679 | HLA-A*11:01_STYGIITSR | 1413 | TMPS7 | ENSG00000176040 | Transmembrane protease serine 7 (EC 3.4.21.-) |
| | | | | | (Matriptase-3) |
| 1680 | HLA-A*31:01_STYGIITSR | 1413 | TMPS7 | ENSG00000176040 | Transmembrane protease serine 7 (EC 3.4.21.-) |
| | | | | | (Matriptase-3) |
| 1681 | HLA-A*11:01_VVADVSNNK | 1414 | TMPS7 | ENSG00000176040 | Transmembrane protease serine 7 (EC 3.4.21.-) |
| | | | | | (Matriptase-3) |
| 1682 | HLA-A*11:01_VVADVSSNNK | 1415 | TMPS7 | ENSG00000176040 | Transmembrane protease serine 7 (EC 3.4.21.-) |
| | | | | | (Matriptase-3) |
| 1683 | HLA-B*18:01_LELATYEL | 1416 | FMR1N | ENSG00000176988 | Fragile X mental retardation 1 neighbor protein |
| | | | | | (Cancer/testis antigen 37)(CT37)(Sarcoma |
| | | | | | antigen NY-SAR-35) |
| 1684 | HLA-A*24:02_NYFIDPVTI | 554 | TR49B | ENSG00000182053 | Putative tripartite motif-containing protein 49B |
| | | | | | (RING finger protein 18B) |
| 1685 | HLA-A*29:02_SIFTGFLLY | 1417 | OTOL1 | ENSG00000182447 | Otolin-1 |
| 1686 | HLA-B*18:01_DESLIYSF | 1418 | TRIML | ENSG00000184108 | Probable E3 ubiquitin-protein ligase TRIML1 |
| | | | | | (EC 2.3.2.27)(RING finger protein 209)(RING- |
| | | | | | type E3 ubiquitin transferase TRIML1)(Tripartite |
| | | | | | motif family-like protein 1) |
| 1687 | HLA-B*44:03_SEDLKSVKY | 1419 | TRIML | ENSG00000184108 | Probable E3 ubiquitin-protein ligase TRIML1 |
| | | | | | (EC 2.3.2.27)(RING finger protein 209)(RING- |
| | | | | | type E3 ubiquitin transferase TRIML1)(Tripartite |
| | | | | | motif family-like protein 1) |
| 1688 | HLA-A*02:07_TLDPATANAYL | 1420 | TRIML | ENSG00000184108 | Probable E3 ubiquitin-protein ligase TRIML1 |
| | | | | | (EC 2.3.2.27)(RING finger protein 209)(RING- |
| | | | | | type E3 ubiquitin transferase TRIML1)(Tripartite |
| | | | | | motif family-like protein 1) |
| 1689 | HLA-A*30:02_VLQSEDEQGSY | 1421 | TRIML | ENSG00000184108 | Probable E3 ubiquitin-protein ligase TRIML1 |
| | | | | | (EC 2.3.2.27)(RING finger protein 209)(RING- |
| | | | | | type E3 ubiquitin transferase TRIML1)(Tripartite |
| | | | | | motif family-like protein 1) |
| 1690 | HLA-A*03:01_RLYSGTARY | 1422 | KCNH7 | ENSG00000184611 | Potassium voltage-gated channel subfamily H |
| | | | | | member 7 (Ether-a-go-go-related gene potassium |
| | | | | | channel 3)(ERG-3)(Eag-related protein 3) |
| | | | | | (Ether-a-go-go-related protein 3)(hERG-3) |
| | | | | | (Voltage-gated potassium channel subunit |
| | | | | | Kv11.3) |
| 1691 | HLA-A*11:01_VTSGEYSLFQK | 1423 | OVCH1 | ENSG00000187950 | Ovochymase-1 (EC 3.4.21.-) |
| 1692 | HLA-A*03:01_NTYASTLYK | 884 | FGF16 | ENSG00000196468 | Fibroblast growth factor 16 (FGF-16) |
| 1693 | HLA-A*03:01_GVHGGILNK | 1424 | PROF3 | ENSG00000196570 | Profilin-3 (Profilin III) |
| 1694 | HLA-A*11:01_GVHGGILNK | 1424 | PROF3 | ENSG00000196570 | Profilin-3 (Profilin III) |
| 1695 | HLA-A*30:02_GGLLGPSHSY | 1425 | DMBX1 | ENSG00000197587 | Diencephalon/mesencephalon homeobox protein |
| | | | | | 1 (Orthodenticle homolog 3)(Paired-like |
| | | | | | homeobox protein DMBX1) |
| 1696 | HLA-A*29:02_TVIDVFYQY | 1426 | HORN | ENSG00000197915 | Homerin |
| 1697 | HLA-A*26:01_EYFPKSVSEY | 1427 | TDR15 | ENSG00000218819 | Tudor domain-containing protein 15 |
| 1698 | HLA-B*35:01_LVASGLATY | 1287 | TDR15 | ENSG00000218819 | Tudor domain-containing protein 15 |
| 1699 | HLA-B*44:03_SETSVSDVNSF | 1155 | TDR15 | ENSG00000218819 | Tudor domain-containing protein 15 |
| 1700 | HLA-A*11:01_ASSQSTPVK | 1428 | MORC1 | ENSG00000114487 | MORC family CW-type zinc finger protein 1 |
| | | | | | (Cancer/testis antigen 33)(CT33) |
| 1701 | HLA-A*11:01_GTMSTISPSK | 1429 | MORC1 | ENSG00000114487 | MORC family CW-type zinc finger protein 1 |
| | | | | | (Cancer/testis antigen 33)(CT33) |
| 1702 | HLA-B*51:01_TPLKWYQSI | 1430 | AMELX | ENSG00000125363 | Amelogenin, X isoform |
| 1703 | HLA-A*02:07_SLPSPGELYAV | 1431 | RNF17 | ENSG00000132972 | RING finger protein 17 (Tudor domain- |
| | | | | | containing protein 4) |
| 1704 | HLA-B*44:02_TEDIGSKGY | 537 | RXFP2 | ENSG00000133105 | Relaxin receptor 2 (G-protein coupled receptor |
| | | | | | 106)(G-protein coupled receptor affecting |
| | | | | | testicular descent)(Leucine-rich repeat- |
| | | | | | containing G-protein coupled receptor 8)(Relaxin |
| | | | | | family peptide receptor 2) |
| 1705 | HLA-C*02:02_TEDIGSKGY | 537 | RXFP2 | ENSG00000133105 | Relaxin receptor 2 (G-protein coupled receptor |
| | | | | | 106)(G-protein coupled receptor affecting |
| | | | | | testicular descent)(Leucine-rich repeat- |
| | | | | | containing G-protein coupled receptor 8)(Relaxin |
| | | | | | family peptide receptor 2) |
| 1706 | HLA-A*11:01_GTEQLTITGK | 1432 | LGSN | ENSG00000146166 | Lengsin (Glutamate-ammonia ligase domain- |
| | | | | | containing protein 1)(Lens glutamine synthase- |
| | | | | | like) |
| 1707 | HLA-A*24:02_LYMQIINFF | 1433 | STRA8 | ENSG00000146857 | Stimulated by retinoic acid gene 8 protein |
| | | | | | homolog |
| 1708 | HLA-A*11:01_SGISQVFQR | 1434 | TRI48 | ENSG00000150244 | Tripartite motif-containing protein 48 (RING |
| | | | | | finger protein 101) |
| 1709 | HLA-B*51:01_DSLPRLTSV | 1435 | CNTP5 | ENSG00000155052 | Contactin-associated protein-like 5 (Cell |
| | | | | | recognition molecule Caspr5) |
| 1710 | HLA-B*44:03_KEVDPAGHSY | 1404 | MAGA8 | ENSG00000156009 | Melanoma-associated antigen 8 (Cancer/testis |
| | | | | | antigen 1.8)(CT1.8)(MAGE-8 antigen) |
| 1711 | HLA-C*02:02_AAASLFEVY | 1167 | OTOP1 | ENSG00000163982 | Otopetrin-1 |
| 1712 | HLA-B*46:01_HAKDIIQSF | 1436 | OTOP1 | ENSG00000163982 | Otopetrin-1 |
| 1713 | HLA-B*18:01_DEEQNLVAF | 380 | PRDM9 | ENSG00000164256 | Histone-lysine N-methyltransferase PRDM9 (EC |
| | | | | | 2.1.1.43)(PR domain zinc finger protein 9)(PR |
| | | | | | domain-containing protein 9) |
| 1714 | HLA-B*44:03_EEAANNGYSW | 1437 | PRDM9 | ENSG00000164256 | Histone-lysine N-methyltransferase PRDM9 (EC |
| | | | | | 2.1.1.43)(PR domain zinc finger protein 9)(PR |
| | | | | | domain-containing protein 9) |
| 1715 | HLA-C*02:02_EEAANNGYSW | 1437 | PRDM9 | ENSG00000164256 | Histone-lysine N-methyltransferase PRDM9 (EC |
| | | | | | 2.1.1.43)(PR domain zinc finger protein 9)(PR |
| | | | | | domain-containing protein 9) |
| 1716 | HLA-B*44:03_EEQNLVAFQY | 382 | PRDM9 | ENSG00000164256 | Histone-lysine N-methyltransferase PRDM9 (EC |
| | | | | | 2.1.1.43)(PR domain zinc finger protein 9)(PR |
| | | | | | domain-containing protein 9) |
| 1717 | HLA-C*16:01_AFSDRTNAL | 1438 | FSHR | ENSG00000170820 | Follicle-stimulating hormone receptor (FSH-R) |
| | | | | | (Follitropin receptor) |
| 1718 | HLA-B*35:01_DAAGFFTVF | 1439 | FSHR | ENSG00000170820 | Follicle-stimulating hormone receptor (FSH-R) |
| | | | | | (Follitropin receptor) |
| 1719 | HLA-B*51:01_DAAGFFTV | 1440 | FSHR | ENSG00000170820 | Follicle-stimulating hormone receptor (FSH-R) |
| | | | | | (Follitropin receptor) |
| 1720 | HLA-B*35:01_FPIFGISSY | 1441 | FSHR | ENSG00000170820 | Follicle-stimulating hormone receptor (FSH-R) |
| | | | | | (Follitropin receptor) |
| 1721 | HLA-C*02:02_AEAALQTLL | 1375 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1722 | HLA-B*35:01_EAISQIASF | 1378 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1723 | HLA-A*11:01_SVADLTESILK | 638 | LDH6B | ENSG00000171989 | L-lactate dehydrogenase A-like 6B (EC 1.1.1.27) |
| 1724 | HLA-A*02:01_TLWEIQNKLKL | 1442 | LDH6B | ENSG00000171989 | L-lactate dehydrogenase A-like 6B (EC 1.1.1.27) |
| 1725 | HLA-A*03:01_VVNQGKGMFK | 411 | CTSRD | ENSG00000174898 | Cation channel sperm-associated protein subunit |
| | | | | | delta (CatSper-delta)(CatSperdelta) |
| | | | | | (Transmembrane protein 146) |
| 1726 | HLA-A*11:01_ASLTTDGSLK | 1443 | TSYL6 | ENSG00000178021 | Testis-specific Y-encoded-like protein 6 (TSPY- |
| | | | | | like protein 6) |
| 1727 | HLA-B*51:01_LAIAGMNTI | 1444 | OTOL1 | ENSG00000182447 | Otolin-1 |
| 1728 | HLA-A*29:02_LYLFGVTKY | 1445 | PSG9 | ENSG00000183668 | Pregnancy-specific beta-1-glycoprotein 9 (PS- |
| | | | | | beta-G-9)(PSBG-9)(Pregnancy-specific |
| | | | | | glycoprotein 9)(PS34)(Pregnancy-specific beta- |
| | | | | | 1 glycoprotein B)(PS-beta-B)(Pregnancy- |
| | | | | | specific beta-1-glycoprotein 11)(PS-beta-G-11) |
| | | | | | (PSBG-11)(Pregnancy-specific glycoprotein 11) |
| | | | | | (Pregnancy-specific glycoprotein 7)(PSG7) |
| 1729 | HLA-B*35:01_HPIGGDVAL | 1446 | PRS38 | ENSG00000185888 | Serine protease 38 (EC 3.4.21.-)(Marapsin-2) |
| 1730 | HLA-B*51:01_FAYIAGHSI | 1447 | GTR7 | ENSG00000197241 | Solute carrier family 2, facilitated glucose |
| | | | | | transporter member 7 (Glucose transporter type |
| | | | | | 7)(GLUT-7) |
| 1731 | HLA-A*02:07_MVDGAVHWL | 1448 | GTR7 | ENSG00000197241 | Solute carrier family 2, facilitated glucose |
| | | | | | transporter member 7 (Glucose transporter type |
| | | | | | 7)(GLUT-7) |
| 1732 | HLA-A*29:02_IVFGDRFDY | 1449 | CP2AD | ENSG00000197838 | Cytochrome P450 2A13 (EC 1.14.14.1) |
| | | | | | (CYPIIA13) |
| 1733 | HLA-B*44:03_EEIFLAKIEKF | 1450 | X6R7K4 | ENSG00000203910 | Chromosome 1 open reading frame 146 |
| | | | | | (Uncharacterized protein C1orf146) |
| 1734 | HLA-A*11:01_STEEIFLAK | 1451 | X6R7K4 | ENSG00000203910 | Chromosome 1 open reading frame 146 |
| | | | | | (Uncharacterized protein C1orf146) |
| 1735 | HLA-A*24:02_IYSNTLQSI | 1452 | GNAT3 | ENSG00000214415 | Guanine nucleotide-binding protein G(t) subunit |
| | | | | | alpha-3 (Gustducin alpha-3 chain) |
| 1736 | HLA-B*18:01_DEKGTIYDY | 1453 | GPX5 | ENSG00000224586 | Epididymal secretory glutathione peroxidase (EC |
| | | | | | 1.11.1.9)(Epididymis-specific glutathione |
| | | | | | peroxidase-like protein)(EGLP)(Glutathione |
| | | | | | peroxidase 5)(GPx-5)(GSHPx-5) |
| 1737 | HLA-B*18:01_NEYVSFKQY | 1454 | GPX5 | ENSG00000224586 | Epididymal secretcay glutathione peroxidase (EC |
| | | | | | 1.11.1.9)(Epididymis-specific glutathione |
| | | | | | peroxidase-like protein)(EGLP)(Glutathione |
| | | | | | peroxidase 5)(GPx-5)(GSHPx-5) |
| 1738 | HLA-B*44:03_NEYVSFKQY | 1454 | GPX5 | ENSG00000224586 | Epididymal secretoty glutathione peroxidase (EC |
| | | | | | 1.11.1.9)(Epididymis-specific glutathione |
| | | | | | peroxidase-like protein)(EGLP)(Glutathione |
| | | | | | peroxidase 5)(GPx-5)(GSHPx-5) |
| 1739 | HLA-A*11:01_GSGLHQVSK | 1455 | E9PBZ7 | ENSG00000242715 | Coiled-coil domain-containing protein 169 |
| 1740 | HLA-B*44:02_IEAELHISY | 254 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD_6p24.1 provirus ancestral Env |
| | | | | | polyprotein)[Cleaved into: Surface protein (SU); |
| | | | | | Transmembrane protein (TM)] |
| 1741 | HLA-A*26:01_DTTHPISYY | 1456 | GGTL2 | ENSG00000100121 | Gamma-glutamyltransferase light chain 2 |
| | | | | | (Gamma-glutamyltransferase-like protein 4) |
| 1742 | HLA-B*51:01_YAHLTNSSI | 1457 | TTLL2 | ENSG00000120440 | Probable tubulin polyglutamylase TTLL2 (EC 6.- |
| | | | | | .-.-)(Testis-specific protein NYD-TSPG) |
| | | | | | (Tubulin--tyro sine ligase-like protein 2) |
| 1743 | HLA-A*11:01_AQNARIFSK | 1458 | CRIS1 | ENSG00000124812 | Cysteine-rich secretoty protein 1 (CRISP-1) |
| | | | | | (AEG-like protein)(ARP)(Acidic epididymal |
| | | | | | glycoprotein homolog) |
| 1744 | HLA-A*02:01_KLVTDLPNV | 1459 | CRIS1 | ENSG00000124812 | Cysteine-rich secretoty protein 1 (CRISP-1) |
| | | | | | (AEG-like protein)(ARP)(Acidic epididymal |
| | | | | | glycoprotein homolog) |
| 1745 | HLA-C*16:01_SEKEPGQQY | 831 | RHXF2 | ENSG00000131721 | Rhox homeobox family member 2 (Paired-like |
| | | | | | homeobox protein PEPP-2)(Testis homeobox |
| | | | | | gene 1) |
| 1746 | HLA-A*29:02_HVVSGVFFY | 1460 | NMUR2 | ENSG00000132911 | Neuromedin-U receptor 2 (NMU-R2)(G-protein |
| | | | | | coupled receptor FM-4)(G-protein coupled |
| | | | | | receptor TGR-1) |
| 1747 | HLA-A*02:07_YVPIFVVGV | 1461 | NMUR2 | ENSG00000132911 | Neuromeclin-U receptor 2 (NMU-R2)(G-protein |
| | | | | | coupled receptor FM-4)(G-protein coupled |
| | | | | | receptor TGR-1) |
| 1748 | HLA-A*02:07_TVTEKIYYL | 695 | F71F1 | ENSG00000135248 | Protein FAM71F1 (Protein FAM137A)(Testis |
| | | | | | development protein NYD-SP18) |
| 1749 | HLA-B*51:01_SAPEIPTI | 1462 | FNDC7 | ENSG00000143107 | Fibronectin type III domain-containing protein 7 |
| 1750 | HLA-B*18:01_DEVQIEVV | 1463 | X6R6V8 | ENSG00000143552 | Nuclear pore membrane glycoprotein 210-like |
| 1751 | HLA-B*35:01_QPIYQQPAY | 1464 | BOLL | ENSG00000152430 | Protein boule-like |
| 1752 | HLA-A*29:02_IFTSATYLY | 1465 | PANX3 | ENSG00000154143 | Pannexin-3 |
| 1753 | HLA-A*30:02_RSHSLVATY | 1466 | PANX3 | ENSG00000154143 | Pannexin-3 |
| 1754 | HLA-A*29:02_YFEFPLLERY | 1467 | PANX3 | ENSG00000154143 | Pannexin-3 |
| 1755 | HLA-A*11:01_GTNIVVLGVEK | 1468 | PSA7L | ENSG00000154611 | Proteasome subunit alpha type-7-like (EC |
| | | | | | 3.4.25.1) |
| 1756 | HLA-A*30:02_AVKSVGEPKY | 1469 | MEIOB | ENSG00000162039 | Meiosis-specific with OB domain-containing |
| | | | | | protein (EC 3.1.-.-) |
| 1757 | HLA-B*18:01_DETESSFAM | 1470 | MEIOB | ENSG00000162039 | Meiosis-specific with OB domain-containing |
| | | | | | protein (EC 3.1.-.-) |
| 1758 | HLA-C*02:02_FASDVRINF | 1471 | MEIOB | ENSG00000162039 | Meiosis-specific with OB domain-containing |
| | | | | | protein (EC 3.1.-.-) |
| 1759 | HLA-C*16:01_FASDVRINF | 1471 | MEIOB | ENSG00000162039 | Meiosis-specific with OB domain-containing |
| | | | | | protein (EC 3.1.-.-) |
| 1760 | HLA-A*03:01_KVIGIVIGK | 1472 | MEIOB | ENSG00000162039 | Meiosis-specific with OB domain-containing |
| | | | | | protein (EC 3.1.-.-) |
| 1761 | HLA-A*11:01_KVIGIVIGK | 1472 | MEIOB | ENSG00000162039 | Meiosis-specific with OB domain-containing |
| | | | | | protein (EC 3.1.-.-) |
| 1762 | HLA-C*04:01_LYDETESSF | 1473 | MEIOB | ENSG00000162039 | Meiosis-specific with OB domain-containing |
| | | | | | protein (EC 3.1.-.-) |
| 1763 | HLA-A*02:07_SLDFKSVFL | 1474 | MEIOB | ENSG00000162039 | Meiosis-specific with OB domain-containing |
| | | | | | protein (EC 3.1.-.-) |
| 1764 | HLA-B*51:01_YAYISTLNI | 1475 | MEIOB | ENSG00000162039 | Meiosis-specific with OB domain-containing |
| | | | | | protein (EC 3.1.-.-) |
| 1765 | HLA-B*44:03_DEYGQELGIKW | 1476 | PRDM9 | ENSG00000164256 | Histone-lysine N-methyltransferase PRDM9 (EC |
| | | | | | 2.1.1.43)(PR domain zinc finger protein 9)(PR |
| | | | | | domain-containing protein 9) |
| 1766 | HLA-B*44:03_SEAVSVLHHW | 1477 | SPT19 | ENSG00000166118 | Spermatogenesis-associated protein 19, |
| | | | | | mitochondrial (Spermatogenic cell-specific gene |
| | | | | | 1 protein)(Spergen-1) |
| 1767 | HLA-A*11:01_STLDLANTLQK | 1478 | MB3L1 | ENSG00000170948 | Methyl-CpG-binding domain protein 3-like 1 |
| | | | | | (MBD 3-like protein 1) |
| 1768 | HLA-B*44:03_QEAAENYRKLF | 1479 | TRIMM | ENSG00000179046 | Probable E3 ubiquitin-protein ligase TRIML2 |
| | | | | | (EC 2.3.2.27)(RING-type E3 ubiquitin |
| | | | | | transferase TRIML2)(SPRY domain-containing |
| | | | | | protein 6)(Tripartite motif family-like protein 2) |
| 1769 | HLA-A*24:02_EYLFDTHTL | 1480 | FTMT | ENSG00000181867 | Ferritin, mitochondrial (EC 1.16.3.1) |
| 1770 | HLA-A*24:02_VYLSMAYYF | 1481 | FTMT | ENSG00000181867 | Ferritin, mitochondrial (EC 1.16.3.1) |
| 1771 | HLA-A*03:01_QTLGIPLTPK | 1482 | H0YFA1 | ENSG00000185958 | Protein FAM186A (Fragment) |
| 1772 | HLA-A*29:02_LFSPITQQLRY | 1483 | CS067 | ENSG00000188032 | UPF0575 protein C19orf67 |
| 1773 | HLA-A*02:01_ALAPLLMTL | 1484 | S22AO | ENSG00000197658 | Solute carrier family 22 member 24 |
| 1774 | HLA-A*30:02_AAHPIGLVY | 1485 | DPRX | ENSG00000204595 | Divergent paired-related homeobox |
| 1775 | HLA-A*30:02_SVTTYTGSY | 530 | CD051 | ENSG00000237136 | Uncharacterized protein C4orf51 |
| 1776 | HLA-B*44:03_AENGLLEKI | 1486 | ASIC5 | ENSG00000256394 | Acid-sensing ion channel 5 (ASIC5)(Amiloride- |
| | | | | | sensitive cation channel 5)(Human intestine |
| | | | | | Na(+) channel)(HINaC) |
| 1777 | HLA-B*44:03_EEIEYPATISY | 1487 | ASIC5 | ENSG00000256394 | Acid-sensing ion channel 5 (ASIC5)(Amiloride- |
| | | | | | sensitive cation channel 5)(Human intestine |
| | | | | | Na(+) channel)(HINaC) |
| 1778 | HLA-B*18:01_IEYPATISY | 1488 | ASIC5 | ENSG00000256394 | Acid-sensing ion channel 5 (ASIC5)(Amiloride- |
| | | | | | sensitive cation channel 5)(Human intestine |
| | | | | | Na(+) channel)(HINaC) |
| 1779 | HLA-B*18:01_MEFPAVTF | 1489 | ASIC5 | ENSG00000256394 | Acid-sensing ion channel 5 (ASIC5)(Amiloride- |
| | | | | | sensitive cation channel 5)(Human intestine |
| | | | | | Na(+) channel)(HINaC) |
| 1780 | HLA-A*11:01_ASQLHTLIK | 1490 | DUXA | ENSG00000258873 | Double homeobox protein A |
| 1781 | HLA-B*44:02_EEQLKILINTF | 1491 | DUXA | ENSG00000258873 | Double homeobox protein A |
| 1782 | HLA-A*31:01_RIQIWFQNR | 1492 | DUXA | ENSG00000258873 | Double homeobox protein A |
| 1783 | HLA-A*31:01_RVQIWFQNR | 1493 | DUXA | ENSG00000258873 | Double homeobox protein A |
| 1784 | HLA-A*11:01_GIADPNQSAK | 1251 | ADAM7 | ENSG00000069206 | Disintegrin and metalloproteinase domain- |
| | | | | | containing protein 7 (ADAM 7)(Sperm |
| | | | | | maturation-related glycoprotein GP-83) |
| 1785 | HLA-A*29:02_GLNQLYFYY | 1494 | TRPC5 | ENSG00000072315 | Short transient receptor potential channels |
| | | | | | (TIpC5)(Transient receptor protein 5)(TRP-5) |
| | | | | | (hTRP-5)(hTRP5) |
| 1786 | HLA-B*35:01_HAISSAGVMY | 1495 | WNT8B | ENSG00000075290 | Protein Wnt-8b |
| 1787 | HLA-A*26:01_EVAQSNSAF | 1496 | SEIL2 | ENSG00000101251 | Protein sel-1 homolog 2 (Suppressor of lin-12- |
| | | | | | like protein 2)(Sel-1L2) |
| 1788 | HLA-A*02:07_ATDSFHTEL | 1497 | DKKL1 | ENSG00000104901 | Dickkopf-like protein 1 (Cancer/testis antigen 34) |
| | | | | | (CT34)(Protein soggy-1)(SGY-1) |
| 1789 | HLA-A*24:02_IYAPPNNRF | 1498 | GCNT7 | ENSG00000124091 | Beta-1,3-galactosyl-O-glycosyl-glycoprotein |
| | | | | | beta-1,6-N-acetylglucosaminyltransferase 7 (EC |
| | | | | | 2.4.1.-) |
| 1790 | HLA-B*51:01_VPAQQPVI | 1499 | AMELX | ENSG00000125363 | Amelogenin, X isoform |
| 1791 | HLA-B*18:01_DESTILHL | 1500 | CCD83 | ENSG00000150676 | Coiled-coil domain-containing protein 83 |
| 1792 | HLA-B*44:03_MEGPFFRDY | 1501 | IZUM2 | ENSG00000161652 | Izumo sperm-egg fusion protein 2 |
| 1793 | HLA-B*35:01_DPAVFQLVY | 1502 | KCNV2 | ENSG00000168263 | Potassium voltage-gated channel subfamily V |
| | | | | | member 2 (Voltage-gated potassium channel |
| | | | | | subunit Kv8.2) |
| 1794 | HLA-A*11:01_GSYSNNSTEK | 1503 | MRO2B | ENSG00000171495 | Maestro heat-like repeat-containing protein |
| | | | | | family member 2B (HEAT repeat-containing |
| | | | | | protein 7B2)(Sperm PKA-interacting factor) |
| | | | | | (SPIF) |
| 1795 | HLA-A*11:01_ATAQKLKKK | 1504 | WDR87 | ENSG00000171804 | WD repeat-containing protein 87 (Testis |
| | | | | | development protein NYD-SP11) |
| 1796 | HLA-A*29:02_GTATLLIVRY | 1505 | UROL1 | ENSG00000177398 | Uromodulin-like 1 (Olfactorin) |
| 1797 | HLA-A*11:01_ATAGARSKVK | 1506 | MAGBA | ENSG00000177689 | Melanoma-associated antigen B10 (MAGE-B10 |
| | | | | | antigen) |
| 1798 | HLA-A*11:01_GTNGFQLLR | 1049 | SG11A | ENSG00000178287 | Sperm-associated antigen 11A (Human |
| | | | | | epididymis-specific protein 2)(He2)(Protein |
| | | | | | EP2)(Sperm antigen HE2) |
| 1799 | HLA-A*02:07_LLPPRTPPYQV | 1050 | SG11A | ENSG00000178287 | Sperm-associated antigen 11A (Human |
| | | | | | epididymis-specific protein 2)(He2)(Protein |
| | | | | | EP2)(Sperm antigen HE2) |
| 1800 | HLA-B*51:01_VPLGIRNTI | 1507 | SG11A | ENSG00000178287 | Sperm-associated antigen 11A (Human |
| | | | | | epididymis-specific protein 2)(He2)(Protein |
| | | | | | EP2)(Sperm antigen HE2) |
| 1801 | HLA-B*46:01_ILRPPVEAY | 1508 | FA71C | ENSG00000180219 | Protein FAM71C |
| 1802 | HLA-A*02:01_VLSAVTPEL | 1509 | SAGE1 | ENSG00000181433 | Sarcoma antigen 1 (Cancer/testis antigen 14) |
| | | | | | (CT14) |
| 1803 | HLA-A*02:07_VVPPWNPQL | 1510 | ADIG | ENSG00000182035 | Adipogenin |
| 1804 | HLA-B*44:03_DEYGNTTLHY | 1039 | D7UEQ8 | ENSG00000183206 | POTE ankyrin domain family member C |
| 1805 | HLA-A*11:01_SQDEILINK | 1511 | D7UEQ8 | ENSG00000183206 | POTE ankyrin domain family member C |
| 1806 | HLA-A*01:01_FSDFGLLWY | 1512 | NALP9 | ENSG00000185792 | NACHT, LRR and PYD domains-containing |
| | | | | | protein 9 (Nucleotide-binding oligomerization |
| | | | | | domain protein 6)(PYRIN and NACHT- |
| | | | | | containing protein 12) |
| 1807 | HLA-B*46:01_NAITGSAF | 1513 | DCC | ENSG00000187323 | Netrin receptor DCC (Colorectal cancer |
| | | | | | suppressor)(Immunoglobulin superfamily DCC |
| | | | | | subclass member 1)(Tumor suppressor protein |
| | | | | | DCC) |
| 1808 | HLA-B*46:01_FAFEKDVEM | 1116 | H9KVA5 | ENSG00000187959 | Putative cleavage and polyadenylation-specificity |
| | | | | | factor subunit 4-like protein |
| 1809 | HLA-B*44:03_SEYPIIFVY | 1514 | SPXN3 | ENSG00000189252 | Sperm protein associated with the nucleus on the |
| | | | | | X chromosome N3 (Nuclear-associated protein |
| | | | | | SPAN-Xn3)(SPANX-N3)(SPANX family |
| | | | | | member N3) |
| 1810 | HLA-A*01:01_GSDFGHSSSY | 1515 | HORN | ENSG00000197915 | Hornerin |
| 1811 | HLA-B*44:02_EEETLKTLY | 1215 | Q5VXJ5 | ENSG00000198765 | Synaptonemal complex protein 1 (Fragment) |
| 1812 | HLA-A*26:01_ETPSETPTY | 1516 | F8W8N9 | ENSG00000204930 | Protein FAM221B (Fragment) |
| 1813 | HLA-A*01:01_ISETPSETPTY | 1517 | F8W8N9 | ENSG00000204930 | Protein FAM221B (Fragment) |
| 1814 | HLA-B*44:03_SETPSETPTY | 1518 | F8W8N9 | ENSG00000204930 | Protein FAM221B (Fragment) |
| 1815 | HLA-B*44:03_EEVARFLTYY | 1519 | 1A1L2 | ENSG00000205126 | Probable inactive 1-aminocyclopropane-1- |
| | | | | | carboxylate synthase-like protein 2 (ACC |
| | | | | | synthase-like protein 2) |
| 1816 | HLA-A*29:02_STLPTTINY | 31 | MAGAC | ENSG00000213401 | Melanoma-associated antigen 12 (Cancer/testis |
| | | | | | antigen 1.12)(CT1.12)(MAGE-12 antigen) |
| | | | | | (MAGE12F antigen) |
| 1817 | HLA-B*44:03_SEAPSLPVVF | 1520 | GRCR1 | ENSG00000215203 | Glutaredoxin domain-containing cysteine-rich |
| | | | | | protein 1 |
| 1818 | HLA-A*30:02_GTGQVSSTY | 1521 | A0A1B0GTN1 | ENSG00000224960 | Putative SMEK homolog 3 |
| 1819 | HLA-A*29:02_HFLFLFLLY | 1522 | PATE3 | ENSG00000236027 | Prostate and testis expressed protein 3 |
| | | | | | (Acrosomal vesicle protein HEL-127)(PATE-like |
| | | | | | protein DJ)(PATE-DJ) |
| 1820 | HLA-B*18:01_MEYLTQAAF | 1523 | BHMG1 | ENSG00000237452 | Basic helix-loop-helix and HMG box domain- |
| | | | | | containing protein 1 |
| 1821 | HLA-B*27:02_GTGTGIAGITK | 253 | SYCY2 | ENSG00000244476 | Syncytin-2 (Endogenous retrovirus group FRD |
| | | | | | member 1)(Envelope polyprotein)(HERV-FRD) |
| | | | | | (HERV-FRD_6p24.1 provirus ancestral Env |
| | | | | | polyprotein)[Cleaved into: Surface protein (SU); |
| | | | | | Transmembrane protein (TM)] |
| 1822 | HLA-B*44:02_VEVGEVKSW | 401 | RFPLB | ENSG00000251258 | Ret finger protein-like 4B (RING finger protein |
| | | | | | 211) |
| 1823 | HLA-A*01:01_WTDGSSYDY | 1524 | CL19A | ENSG00000261210 | C-type lectin domain family 19 member A |
| 1824 | HLA-B*44:03_EEFLDGEHW | 1525 | CALR3 | ENSG00000269058 | Calreticulin-3 (Calreticulin-2)(Calsperin) |
| 1825 | HLA-B*44:03_SEFENIGAIGL | 1526 | CALR3 | ENSG00000269058 | Calreticulin-3 (Calreticulin-2)(Calsperin) |
| 1826 | HLA-A*02:01_GLSEVISVV | 1527 | TRPC7 | ENSG00000069018 | Short transient receptor potential channel 7 |
| | | | | | (TipC7)(Transient receptor protein 7)(TRP-7) |
| | | | | | (hTRP7) |
| 1827 | HLA-B*44:03_IETEFKNDY | 886 | TRPC7 | ENSG00000069018 | Short transient receptor potential channel 7 |
| | | | | | (TipC7)(Transient receptor protein 7)(TRP-7) |
| | | | | | (hTRP7) |
| 1828 | HLA-A*24:02_IYANISGHL | 1528 | ADAM2 | ENSG00000104755 | Disintegrin and metalloproteinase domain- |
| | | | | | containing protein 2 (ADAM 2)(Cancer/testis |
| | | | | | antigen 15)(CT15)(Fertilin subunit beta)(PH- |
| | | | | | 30)(PH30)(PH30-beta) |
| 1829 | HLA-A*1101_AVYENELVATR | 1529 | ZP4 | ENSG00000116996 | Zona pellucida sperm-binding protein 4 (Zona |
| | | | | | pellucida glycoprotein 4)(Zp-4)(Zona pellucida |
| | | | | | protein B)[Cleaved into: Processed zona |
| | | | | | pellucida sperm-binding protein 41 |
| 1830 | HLA-B*44:03_NEIVATIKF | 1530 | SACA1 | ENSG00000118434 | Sperm acrosome membrane-associated protein 1 |
| | | | | | (Sperm acrosomal membrane-associated protein |
| | | | | | 32) |
| 1831 | HLA-A*30:02_AQNARIFSKY | 1531 | CRIS1 | ENSG00000124812 | Cysteine-rich secretory protein 1 (CRISP-1) |
| | | | | | (AEG-like protein)(ARP)(Acidic epididymal |
| | | | | | glycoprotein homotog) |
| 1832 | HLA-B*44:03_EEIVNIHNAL | 1532 | CRIS1 | ENSG00000124812 | Cysteine-rich secretory protein 1 (CRISP-1) |
| | | | | | (AEG-like protein)(ARP)(Acidic epididymal |
| | | | | | glycoprotein homolog) |
| 1833 | HLA-A*03:01_RVVPPASNMLK | 1395 | CRIS1 | ENSG00000124812 | Cysteine-rich secretory protein 1 (CRISP-1) |
| | | | | | (AEG-like protein)(ARP)(Acidic epididymal |
| | | | | | glycoprotein homolog) |
| 1834 | HLA-A*03:01_AINLVTKGINK | 1533 | BAFL | ENSG00000125888 | Barrier-to-autointegration factor-like protein |
| | | | | | (BAF-L)(Barrier-to-autointegration factor 2) |
| 1835 | HLA-A*11:01_AINLVTKGINK | 1533 | BAFL | ENSG00000125888 | Barrier-to-autointegration factor-like protein |
| | | | | | (BAF-L)(Barrier-to-autointegration factor 2) |
| 1836 | HLA-A*02:07_WVDGISHEL | 1534 | BAFL | ENSG00000125888 | Barrier-to-autointegration factor-like protein |
| | | | | | (BAF-L)(Barrier-to-autointegration factor 2) |
| 1837 | HLA-A*02:07_KLDQTTMNV | 1535 | MMP20 | ENSG00000137674 | Matrix metalloproteinase-20 (MMP-20)(EC |
| | | | | | 3.4.24.-)(Enamel metalloproteinase) |
| | | | | | (Enamelysin) |
| 1838 | HLA-A*29:02_YFFSGPKTY | 1536 | MMP20 | ENSG00000137674 | Matrix metalloproteinase-20 (MMP-20)(EC |
| | | | | | 3.4.24.-)(Enamel metalloproteinase) |
| | | | | | (Enamelysin) |
| 1839 | HLA-A*02:01_TLIETTAEA | 1537 | NDST4 | ENSG00000138653 | Bifunctional heparan sulfate N-deacetylase/N- |
| | | | | | sulfotransferase 4 (EC 2.8.2.8)(Glucosaminyl N- |
| | | | | | deacetylase/N-sulfotransferase 4)(NDST-4)(N- |
| | | | | | heparan sulfate sulfotransferase 4)(N-HSST 4) |
| | | | | | [Includes: Heparan sulfate N-deacetylase 4 (EC |
| | | | | | 3.-.-.-); Heparan sulfate N-sulfotransferase 4 (EC |
| | | | | | 2.8.2.-)] |
| 1840 | HLA-A*02:01_FLWRGNVVL | 1538 | TRI43 | ENSG00000144015 | Tripartite motif-containing protein 43 |
| 1841 | HLA-B*18:01_QEVITTVY | 1539 | ZAN | ENSG00000146839 | Zonadhesin |
| 1842 | HLA-B*44:02_AEQLVKTGW | 1348 | OCSTP | ENSG00000149635 | Osteoclast stimulatory transmembrane protein |
| | | | | | (OC-STAMP) |
| 1843 | HLA-A*11:01_AVDIVSQSK | 1540 | PO4F2 | ENSG00000151615 | POU domain, class 4, transcription factor 2 |
| | | | | | (Brain-specific homeobox/POU domain protein |
| | | | | | 3B)(Brain-3B)(Brn-3B) |
| 1844 | HLA-B*18:01_EESSINYTF | 1329 | J3KNE0 | ENSG00000153165 | RanBP2-like and GRIP domain-containing |
| | | | | | protein 3 |
| 1845 | HLA-A*02:07_KLPVPLESV | 1541 | J3KNE0 | ENSG00000153165 | RanBP2-like and GRIP domain-containing |
| | | | | | protein 3 |
| 1846 | HLA-B*35:01_TAFGDLEVF | 1542 | ASZ1 | ENSG00000154438 | Ankyrin repeat, SAM and basic leucine zipper |
| | | | | | domain-containing protein 1 (Ankyrin-like |
| | | | | | protein 1)(Germ cell-specific ankyrin, SAM and |
| | | | | | basic leucine zipper domain-containing protein) |
| 1847 | HLA-A*01:01_KTELETALYY | 1543 | GG6L2 | ENSG00000174450 | Golgin subfamily A member 6-like protein 2 |
| 1848 | HLA-B*18:01_TELETALYY | 1544 | GG6L2 | ENSG00000174450 | Golgin subfamily A member 6-like protein 2 |
| 1849 | HLA-A*26:01_ESIPESSLY | 1545 | UROL1 | ENSG00000177398 | Uromodulin-like 1 (Olfactorin) |
| 1850 | HLA-A*02:01_ALAESVAQL | 1546 | A3LT2 | ENSG00000184389 | Alpha-1,3-galactosyltransferase 2 (EC 2.4.1.87) |
| | | | | | (Isoglobotriaosylceramide synthase)(iGb3 |
| | | | | | synthase)(iGb3S) |
| 1851 | HLA-B*51:01_LAYLVGQSI | 1547 | PIWL3 | ENSG00000184571 | Piwi-like protein 3 |
| 1852 | HLA-A*29:02_IVLPVWLNY | 1548 | VHLL | ENSG00000189030 | Von Hippel-Lindau-like protein (VHL-like |
| | | | | | protein)(VLP) |
| 1853 | HLA-A*29:02_AAHPIGLVY | 1485 | DPRX | ENSG00000204595 | Divergent paired-related homeobox |
| 1854 | HLA-B*35:01_EPLSVTAKY | 874 | VCX3B | ENSG00000205642 | Variable charge X-linked protein 3B (Variably |
| | | | | | charged protein X-C)(VCX-C) |
| 1855 | HLA-A*11:01_ATMGKLASK | 1549 | LEUTX | ENSG00000213921 | Leucine-twenty homeobox |
| 1856 | HLA-A*29:02_IITDLSLYY | 1550 | M4A18 | ENSG00000214782 | Membrane-spanning 4-domains subfamily A |
| | | | | | member 18 |
| 1857 | HLA-A*31:01_QSQPIGYQR | 1551 | M4A18 | ENSG00000214782 | Membrane-spanning 4-domains subfamily A |
| | | | | | member 18 |
| 1858 | HLA-A*30:02_KNIALNGEY | 1552 | GRCR1 | ENSG00000215203 | Glutaredoxin domain-containing cysteine-rich |
| | | | | | protein 1 |
| 1859 | HLA-A*29:02_LFLFGVTKY | 1313 | PSG11 | ENSG00000243130 | Pregnancy-specific beta-1-glycoprotein 11 (PS- |
| | | | | | beta-G-11)(PSBG-11)(Pregnancy-specific |
| | | | | | glycoprotein 11)(Pregnancy-specific beta-1- |
| | | | | | glycoprotein 13)(PS-beta-G-13)(PSBG-13) |
| | | | | | (Pregnancy-specific glycoprotein 13) |
| 1860 | HLA-B*51:01_YPKLPMPYI | 1553 | PSG11 | ENSG00000243130 | Pregnancy-specific beta-1-glycoprotein 11 (PS- |
| | | | | | beta-G-11)(PSBG-11)(Pregnancy-specific |
| | | | | | glycoprotein 11)(Pregnancy-specific beta-1- |
| | | | | | glycoprotein 13)(PS-beta-G-13)(PSBG-13) |
| | | | | | (Pregnancy-specific glycoprotein 13) |
| 1861 | HLA-A*24:02_VYIPGSNATL | 1554 | PCDG8 | ENSG00000253767 | Protocadherin gamma-A8 (PCDH-gamma-A8) |
| 1862 | HLA-A*03:01_KVYAENGLLEK | 1555 | ASIC5 | ENSG00000256394 | Acid-sensing ion channel 5 (ASIC5)(Amiloride- |
| | | | | | sensitive cation channel 5)(Human intestine |
| | | | | | Na(+) channel)(HINaC) |
| 1863 | HLA-B*44:03_AEFIESGQY | 1556 | TAF7L | ENSG00000102387 | Transcription initiation factor TFIID subunit 7- |
| | | | | | like (Cancer/testis antigen 40)(CT40)(RNA |
| | | | | | polymerase II TBP-associated factor subunit Q) |
| | | | | | (TATA box-binding protein-associated factor 50 |
| | | | | | kDa)(Transcription initiation factor TFIID 50 |
| | | | | | kDa subunit) |
| 1864 | HLA-A*11:01_ASTDPNIVRK | 1557 | TAF7L | ENSG00000102387 | Transcription initiation factor TFIID subunit 7- |
| | | | | | like (Cancer/testis antigen 40)(CT40)(RNA |
| | | | | | polymerase II TBP-associated factor subunit Q) |
| | | | | | (TATA box-binding protein-associated factor 50 |
| | | | | | kDa)(Transcription initiation factor TFIID 50 |
| | | | | | kDa subunit) |
| 1865 | HLA-A*11:01_STDPNIVRKK | 1558 | TAF7L | ENSG00000102387 | Transcription initiation factor TFIID subunit 7- |
| | | | | | like (Cancer/testis antigen 40)(CT40)(RNA |
| | | | | | polymerase II TBP-associated factor subunit Q) |
| | | | | | (TATA box-binding protein-associated factor 50 |
| | | | | | kDa)(Transcription initiation factor TFIID 50 |
| | | | | | kDa subunit) |
| 1866 | HLA-A*11:01_STDPNIVRK | 1559 | TAF7L | ENSG00000102387 | Transcription initiation factor TFIID subunit 7- |
| | | | | | like (Cancer/testis antigen 40)(CT40)(RNA |
| | | | | | polymerase II TBP-associated factor subunit Q) |
| | | | | | (TATA box-binding protein-associated factor 50 |
| | | | | | kDa)(Transcription initiation factor TFIID 50 |
| | | | | | kDa subunit) |
| 1867 | HLA-A*03:01_AVSPPASNMLK | 1560 | CRIS2 | ENSG00000124490 | Cysteine-rich secretory protein 2 (CRISP-2) |
| | | | | | (Cancer/testis antigen 36)(CT36)(Testis-specific |
| | | | | | protein TPX-1) |
| 1868 | HLA-A*11:01_AVSPPASNMLK | 1560 | CRIS2 | ENSG00000124490 | Cysteine-rich secretory protein 2 (CRISP-2) |
| | | | | | (Cancer/testis antigen 36)(CT36)(Testis-specific |
| | | | | | protein TPX-1) |
| 1869 | HLA-B*18:01_DEILDFVY | 1561 | CRIS2 | ENSG00000124490 | Cysteine-rich secretory protein 2 (CRISP-2) |
| | | | | | (Cancer/testis antigen 36)(CT36)(Testis-specific |
| | | | | | protein TPX-1) |
| 1870 | HLA-B*51:01_DPTSWSSAI | 1562 | CRIS2 | ENSG00000124490 | Cysteine-rich secretory protein 2 (CRISP-2) |
| | | | | | (Cancer/testis antigen 36)(CT36)(Testis-specific |
| | | | | | protein TPX-1) |
| 1871 | HLA-B*35:01_LPAEGKDPAF | 1563 | CRIS2 | ENSG00000124490 | Cysteine-rich secretory protein 2 (CRISP-2) |
| | | | | | (Cancer/testis antigen 36)(CT36)(Testis-specific |
| | | | | | protein TPX-1) |
| 1872 | HLA-B*51:01_LPVLFLVTV | 1564 | CRIS2 | ENSG00000124490 | Cysteine-rich secretory protein 2 (CRISP-2) |
| | | | | | (Cancer/testis antigen 36)(CT36)(Testis-specific |
| | | | | | protein TPX-1) |
| 1873 | HLA-C*01:02_VSPPASNML | 1565 | CRIS2 | ENSG00000124490 | Cysteine-rich secretory protein 2 (CRISP-2) |
| | | | | | (Cancer/testis antigen 36)(CT36)(Testis-specific |
| | | | | | protein TPX-1) |
| 1874 | HLA-C*02:02_AEFIESGQY | 1556 | TAF7L | ENSG00000102387 | Transcription initiation factor TFIID subunit 7- |
| | | | | | like (Cancer/testis antigen 40)(CT40)(RNA |
| | | | | | polymerase II TBP-associated factor subunit Q) |
| | | | | | (TATA box-binding protein-associated factor 50 |
| | | | | | kDa)(Transcription initiation factor TFIID 50 |
| | | | | | kDa subunit) |
| 1875 | HLA-A*03:01_STDPNIVRKK | 1558 | TAF7L | ENSG00000102387 | Transcription initiation factor TFIID subunit 7- |
| | | | | | like (Cancer/testis antigen 40)(CT40)(RNA |
| | | | | | polymerase II TBP-associated factor subunit Q) |
| | | | | | (TATA box-binding protein-associated factor 50 |
| | | | | | kDa)(Transcription initiation factor TFIID 50 |
| | | | | | kDa subunit) |
| 1876 | HLA-B*44:03_REVTTNAQRW | 1566 | CRIS2 | ENSG00000124490 | Cysteine-rich secretory protein 2 (CRISP-2) |
| | | | | | (Cancer/testis antigen 36)(CT36)(Testis-specific |
| | | | | | protein TPX-1) |
| Target | Gene Name | Gene ID | Peptide | SEQ ID NO | HLA |
| 1877 | AFP | ENSG00000081051 | AADIIIGHL | 1567 | HLA-A*02:07 |
| 1878 | AFP | ENSG00000081051 | AADIIIGHL | 1567 | HLA-A*68:02 |
| 1879 | AFP | ENSG00000081051 | AADIIIGHL | 1567 | HLA-B*38:01 |
| 1880 | AFP | ENSG00000081051 | AADIIIGHL | 1567 | HLA-B*40:01 |
| 1881 | AFP | ENSG00000081051 | AADIIIGHL | 1567 | HLA-C*02:02 |
| 1882 | AFP | ENSG00000081051 | AADIIIGHL | 1567 | HLA-C*05:01 |
| 1883 | AFP | ENSG00000081051 | AATVTKELR | 1568 | HLA-A*68:01 |
| 1884 | AFP | ENSG00000081051 | AATVTKELR | 1568 | HLA-C*07:06 |
| 1885 | AFP | ENSG00000081051 | AATVTKEL | 1569 | HLA-A*32:01 |
| 1886 | AFP | ENSG00000081051 | AATVTKEL | 1569 | HLA-B*08:01 |
| 1887 | AFP | ENSG00000081051 | AATVTKEL | 1569 | HLA-B*46:01 |
| 1888 | AFP | ENSG00000081051 | AATVTKEL | 1569 | HLA-B*58:01 |
| 1889 | AFP | ENSG00000081051 | AATVTKEL | 1569 | HLA-C*01:02 |
| 1890 | AFP | ENSG00000081051 | AATVTKEL | 1569 | HLA-C*03:03 |
| 1891 | AFP | ENSG00000081051 | AATVTKEL | 1569 | HLA-C*03:04 |
| 1892 | AFP | ENSG00000081051 | AATVTKEL | 1569 | HLA-C*05:01 |
| 1893 | AFP | ENSG00000081051 | AATVTKEL | 1569 | HLA-C*12:03 |
| 1894 | AFP | ENSG00000081051 | AATVTKEL | 1569 | HLA-C*14:02 |
| 1895 | AFP | ENSG00000081051 | AATVTKEL | 1569 | HLA-C*16:01 |
| 1896 | AFP | ENSG00000081051 | AATVTKEL | 1569 | HLA-C*16:02 |
| 1897 | AFP | ENSG00000081051 | AATVTKEL | 1569 | HLA-C*16:04 |
| 1898 | AFP | ENSG00000081051 | ADFSGLLEK | 1570 | HLA-A*03:01 |
| 1899 | AFP | ENSG00000081051 | ADFSGLLEK | 1570 | HLA-A*03:02 |
| 1900 | AFP | ENSG00000081051 | ADFSGLLEK | 1570 | HLA-A*11:01 |
| 1901 | AFP | ENSG00000081051 | ADFSGLLEK | 1570 | HLA-B*27:02 |
| 1902 | AFP | ENSG00000081051 | ADFSGLLEK | 1570 | HLA-B*27:05 |
| 1903 | AFP | ENSG00000081051 | ADIIIGHL | 1571 | HLA-A*30:01 |
| 1904 | AFP | ENSG00000081051 | ADIIIGHL | 1571 | HLA-B*37:01 |
| 1905 | AFP | ENSG00000081051 | ADIIIGHL | 1571 | HLA-B*40:02 |
| 1906 | AFP | ENSG00000081051 | ADIIIGHL | 1571 | HLA-B*44:02 |
| 1907 | AFP | ENSG00000081051 | ADLATIFF | 1572 | HLA-B*37:01 |
| 1908 | AFP | ENSG00000081051 | AEEGQKLI | 1573 | HLA-B*44:02 |
| 1909 | AFP | ENSG00000081051 | AEEGQKLI | 1573 | HLA-B*44:03 |
| 1910 | AFP | ENSG00000081051 | AEEGQKLI | 1573 | HLA-B*49:01 |
| 1911 | AFP | ENSG00000081051 | AEISLADLATI | 1574 | HLA-A*30:01 |
| 1912 | AFP | ENSG00000081051 | AEISLADLATI | 1574 | HLA-B*40:01 |
| 1913 | AFP | ENSG00000081051 | AEISLADLATI | 1574 | HLA-B*44:02 |
| 1914 | AFP | ENSG00000081051 | AEISLADLATI | 1574 | HLA-B*44:03 |
| 1915 | AFP | ENSG00000081051 | AEISLADLATI | 1574 | HLA-B*49:01 |
| 1916 | AFP | ENSG00000081051 | AEISLADLA | 1575 | HLA-A*30:01 |
| 1917 | AFP | ENSG00000081051 | AEISLADLA | 1575 | HLA-B*40:01 |
| 1918 | AFP | ENSG00000081051 | AEISLADLA | 1575 | HLA-B*40:02 |
| 1919 | AFP | ENSG00000081051 | AEISLADLA | 1575 | HLA-B*49:01 |
| 1920 | AFP | ENSG00000081051 | AEISLADL | 1576 | HLA-A*30:01 |
| 1921 | AFP | ENSG00000081051 | AEISLADL | 1576 | HLA-B*37:01 |
| 1922 | AFP | ENSG00000081051 | AEISLADL | 1576 | HLA-B*40:01 |
| 1923 | AFP | ENSG00000081051 | AEISLADL | 1576 | HLA-B*44:03 |
| 1924 | AFP | ENSG00000081051 | AEISLADL | 1576 | HLA-B*49:01 |
| 1925 | AFP | ENSG00000081051 | AENAVECF | 1577 | HLA-A*30:01 |
| 1926 | AFP | ENSG00000081051 | AENAVECF | 1577 | HLA-B*18:01 |
| 1927 | AFP | ENSG00000081051 | AENAVECF | 1577 | HLA-B*27:02 |
| 1928 | AFP | ENSG00000081051 | AENAVECF | 1577 | HLA-B*37:01 |
| 1929 | AFP | ENSG00000081051 | AENAVECF | 1577 | HLA-B*44:02 |
| 1930 | AFP | ENSG00000081051 | AENAVECF | 1577 | HLA-B*44:03 |
| 1931 | AFP | ENSG00000081051 | AENAVECF | 1577 | HLA-C*16:04 |
| 1932 | AFP | ENSG00000081051 | AENDEKPEGL | 1578 | HLA-A*30:01 |
| 1933 | AFP | ENSG00000081051 | AENDEKPEGL | 1578 | HLA-B*27:02 |
| 1934 | AFP | ENSG00000081051 | AENDEKPEGL | 1578 | HLA-B*40:01 |
| 1935 | AFP | ENSG00000081051 | AENDEKPEGL | 1578 | HLA-B*44:02 |
| 1936 | AFP | ENSG00000081051 | AENDEKPEGL | 1578 | HLA-B*44:03 |
| 1937 | AFP | ENSG00000081051 | AENDEKPEGL | 1578 | HLA-C*16:04 |
| 1938 | AFP | ENSG00000081051 | AFSDDKFIF | 1579 | HLA-A*23:01 |
| 1939 | AFP | ENSG00000081051 | AFSDDKFIF | 1579 | HLA-A*29:02 |
| 1940 | AFP | ENSG00000081051 | ALQTMKQEF | 1580 | HLA-A*32:01 |
| 1941 | AFP | ENSG00000081051 | ALQTMKQEF | 1580 | HLA-B*15:01 |
| 1942 | AFP | ENSG00000081051 | ALQTMKQEF | 1580 | HLA-B*37:01 |
| 1943 | AFP | ENSG00000081051 | ALQTMKQEF | 1580 | HLA-C*14:02 |
| 1944 | AFP | ENSG00000081051 | ALQTMKQEF | 1580 | HLA-C*16:01 |
| 1945 | AFP | ENSG00000081051 | APQLTSSELMA | 1581 | HLA-B*56:01 |
| 1946 | AFP | ENSG00000081051 | APQLTSSELM | 1582 | HLA-B*07:02 |
| 1947 | AFP | ENSG00000081051 | APQLTSSEL | 1583 | HLA-B*07:02 |
| 1948 | AFP | ENSG00000081051 | APQLTSSEL | 1583 | HLA-B*35:01 |
| 1949 | AFP | ENSG00000081051 | APQLTSSEL | 1583 | HLA-B*35:03 |
| 1950 | AFP | ENSG00000081051 | APQLTSSEL | 1583 | HLA-B*55:01 |
| 1951 | AFP | ENSG00000081051 | APQLTSSEL | 1583 | HLA-B*56:01 |
| 1952 | AFP | ENSG00000081051 | APQLTSSEL | 1583 | HLA-C*01:02 |
| 1953 | AFP | ENSG00000081051 | APQLTSSEL | 1583 | HLA-C*07:02 |
| 1954 | AFP | ENSG00000081051 | APQLTSSEL | 1583 | HLA-C*14:02 |
| 1955 | AFP | ENSG00000081051 | APTILLWAA | 1584 | HLA-B*54:01 |
| 1956 | AFP | ENSG00000081051 | APTILLWAA | 1584 | HLA-B*55:01 |
| 1957 | AFP | ENSG00000081051 | APTILLWAA | 1584 | HLA-B*56:01 |
| 1958 | AFP | ENSG00000081051 | AQFVQEATYK | 1585 | HLA-A*03:01 |
| 1959 | AFP | ENSG00000081051 | AQFVQEATYK | 1585 | HLA-A*03:02 |
| 1960 | AFP | ENSG00000081051 | AQFVQEATYK | 1585 | HLA-A*11:01 |
| 1961 | AFP | ENSG00000081051 | AQFVQEATYK | 1585 | HLA-B*I3:02 |
| 1962 | AFP | ENSG00000081051 | AQFVQEATYK | 1585 | HLA-B*27:02 |
| 1963 | AFP | ENSG00000081051 | AQFVQEATYK | 1585 | HLA-B*27:05 |
| 1964 | AFP | ENSG00000081051 | AQFVQEATY | 1586 | HLA-A*25:01 |
| 1965 | AFP | ENSG00000081051 | AQFVQEATY | 1586 | HLA-A*26:01 |
| 1966 | AFP | ENSG00000081051 | AQFVQEATY | 1586 | HLA-A*29:02 |
| 1967 | AFP | ENSG00000081051 | AQFVQEATY | 1586 | HLA-A*30:02 |
| 1968 | AFP | ENSG00000081051 | AQFVQEATY | 1586 | HLA-A*32:01 |
| 1969 | AFP | ENSG00000081051 | AQFVQEATY | 1586 | HLA-B*13:02 |
| 1970 | AFP | ENSG00000081051 | AQFVQEATY | 1586 | HLA-B*15:01 |
| 1971 | AFP | ENSG00000081051 | AQFVQEATY | 1586 | HLA-B*15:03 |
| 1972 | AFP | ENSG00000081051 | AQFVQEATY | 1586 | HLA-B*18:01 |
| 1973 | AFP | ENSG00000081051 | AQFVQEATY | 1586 | HLA-B*27:02 |
| 1974 | AFP | ENSG00000081051 | AQFVQEATY | 1586 | HLA-B*27:05 |
| 1975 | AFP | ENSG00000081051 | AQFVQEATY | 1586 | HLA-B*35:01 |
| 1976 | AFP | ENSG00000081051 | AQFVQEATY | 1586 | HLA-B*37:01 |
| 1977 | AFP | ENSG00000081051 | AQFVQEATY | 1586 | HLA-B*39:01 |
| 1978 | AFP | ENSG00000081051 | AQFVQEATY | 1586 | HLA-B*44:02 |
| 1979 | AFP | ENSG00000081051 | AQFVQEATY | 1586 | HLA-B*44:03 |
| 1980 | AFP | ENSG00000081051 | AQFVQEATY | 1586 | HLA-B*46:01 |
| 1981 | AFP | ENSG00000081051 | AQFVQEATY | 1586 | HLA-B*58:01 |
| 1982 | AFP | ENSG00000081051 | AQFVQEATY | 1586 | HLA-C*02:02 |
| 1983 | AFP | ENSG00000081051 | AQFVQEATY | 1586 | HLA-C*07:04 |
| 1984 | AFP | ENSG00000081051 | AQFVQEATY | 1586 | HLA-C*12:03 |
| 1985 | AFP | ENSG00000081051 | AQFVQEATY | 1586 | HLA-C*14:02 |
| 1986 | AFP | ENSG00000081051 | AQFVQEATY | 1586 | HLA-C*16:01 |
| 1987 | AFP | ENSG00000081051 | AQFVQEATY | 1586 | HLA-C*16:02 |
| 1988 | AFP | ENSG00000081051 | AQFVQEATY | 1586 | HLA-C*16:04 |
| 1989 | AFP | ENSG00000081051 | AQFVQEAT | 1587 | HLA-B*13:02 |
| 1990 | AFP | ENSG00000081051 | AQFVQEAT | 1587 | HLA-B*15:01 |
| 1991 | AFP | ENSG00000081051 | AQGVALQTM | 1588 | HLA-B*13:02 |
| 1992 | AFP | ENSG00000081051 | AQGVALQTM | 1588 | HLA-B*15:01 |
| 1993 | AFP | ENSG00000081051 | AQGVALQTM | 1588 | HLA-B*15:03 |
| 1994 | AFP | ENSG00000081051 | AQGVALQTM | 1588 | HLA-B*27:05 |
| 1995 | AFP | ENSG00000081051 | AQGVALQTM | 1588 | HLA-B*37:01 |
| 1996 | AFP | ENSG00000081051 | AQGVALQTM | 1588 | HLA-C*01:02 |
| 1997 | AFP | ENSG00000081051 | AQGVALQTM | 1588 | HLA-C*07:04 |
| 1998 | AFP | ENSG00000081051 | AQGVALQTM | 1588 | HLA-C*14:02 |
| 1999 | AFP | ENSG00000081051 | AQGVALQT | 1589 | HLA-B*13:02 |
| 2000 | AFP | ENSG00000081051 | ASFVHEYSR | 1590 | HLA-A*11:01 |
| 2001 | AFP | ENSG00000081051 | ASFVHEYSR | 1590 | HLA-A*31:01 |
| 2002 | AFP | ENSG00000081051 | ASFVHEYSR | 1590 | HLA-B*57:01 |
| 2003 | AFP | ENSG00000081051 | ASFVHEYSR | 1590 | HLA-C*07:06 |
| 2004 | AFP | ENSG00000081051 | ATIFFAQFV | 1591 | HLA-A*68:02 |
| 2005 | AFP | ENSG00000081051 | ATYKEVSKMVK | 1592 | HLA-A*03:01 |
| 2006 | AFP | ENSG00000081051 | ATYKEVSKMVK | 1592 | HLA-A*03:02 |
| 2007 | AFP | ENSG00000081051 | ATYKEVSKMVK | 1592 | HLA-A*11:01 |
| 2008 | AFP | ENSG00000081051 | ATYKEVSKMVK | 1592 | HLA-A*31:01 |
| 2009 | AFP | ENSG00000081051 | ATYKEVSKM | 1593 | HLA-A*03:01 |
| 2010 | AFP | ENSG00000081051 | ATYKEVSKM | 1593 | HLA-A*03:02 |
| 2011 | AFP | ENSG00000081051 | ATYKEVSKM | 1593 | HLA-A*11:01 |
| 2012 | AFP | ENSG00000081051 | ATYKEVSKM | 1593 | HLA-A*25:01 |
| 2013 | AFP | ENSG00000081051 | ATYKEVSKM | 1593 | HLA-A*26:01 |
| 2014 | AFP | ENSG00000081051 | ATYKEVSKM | 1593 | HLA-A*31:01 |
| 2015 | AFP | ENSG00000081051 | ATYKEVSKM | 1593 | HLA-A*32:01 |
| 2016 | AFP | ENSG00000081051 | ATYKEVSKM | 1593 | HLA-B*15:01 |
| 2017 | AFP | ENSG00000081051 | ATYKEVSKM | 1593 | HLA-B*40:02 |
| 2018 | AFP | ENSG00000081051 | ATYKEVSKM | 1593 | HLA-B*58:01 |
| 2019 | AFP | ENSG00000081051 | ATYKEVSKM | 1593 | HLA-C*02:02 |
| 2020 | AFP | ENSG00000081051 | ATYKEVSKM | 1593 | HLA-C*12:03 |
| 2021 | AFP | ENSG00000081051 | ATYKEVSKM | 1593 | HLA-C*16:02 |
| 2022 | AFP | ENSG00000081051 | ATYKEVSK | 1594 | HLA-A*11:01 |
| 2023 | AFP | ENSG00000081051 | AVIADFSGL | 1595 | HLA-A*02:07 |
| 2024 | AFP | ENSG00000081051 | AVIADFSGL | 1595 | HLA-A*25:01 |
| 2025 | AFP | ENSG00000081051 | AVIADFSGL | 1595 | HLA-A*26:01 |
| 2026 | AFP | ENSG00000081051 | AVIADFSGL | 1595 | HLA-B*15:01 |
| 2027 | AFP | ENSG00000081051 | AVIADFSGL | 1595 | HLA-B*40:01 |
| 2028 | AFP | ENSG00000081051 | AVIADFSGL | 1595 | HLA-B*46:01 |
| 2029 | AFP | ENSG00000081051 | AVIADFSGL | 1595 | HLA-B*58:01 |
| 2030 | AFP | ENSG00000081051 | AVIADFSGL | 1595 | HLA-C*01:02 |
| 2031 | AFP | ENSG00000081051 | AVIADFSGL | 1595 | HLA-C*03:03 |
| 2032 | AFP | ENSG00000081051 | AVIADFSGL | 1595 | HLA-C*03:04 |
| 2033 | AFP | ENSG00000081051 | AVIADFSGL | 1595 | HLA-C*16:04 |
| 2034 | AFP | ENSG00000081051 | AVMKNFGTR | 1596 | HLA-A*03:01 |
| 2035 | AFP | ENSG00000081051 | AVMKNFGTR | 1596 | HLA-A*03:02 |
| 2036 | AFP | ENSG00000081051 | AVMKNFGTR | 1596 | HLA-A*11:01 |
| 2037 | AFP | ENSG00000081051 | AVMKNFGTR | 1596 | HLA-A*31:01 |
| 2038 | AFP | ENSG00000081051 | AVMKNFGTR | 1596 | HLA-A*33:03 |
| 2039 | AFP | ENSG00000081051 | AVMKNFGTR | 1596 | HLA-A*68:01 |
| 2040 | AFP | ENSG00000081051 | AVMKNFGTR | 1596 | HLA-C*07:06 |
| 2041 | AFP | ENSG00000081051 | AVSVILRVAK | 1597 | HLA-A*03:01 |
| 2042 | AFP | ENSG00000081051 | AVSVILRVAK | 1597 | HLA-A*03:02 |
| 2043 | AFP | ENSG00000081051 | AVSVILRVAK | 1597 | HLA-A*11:01 |
| 2044 | AFP | ENSG00000081051 | AVSVILRVA | 1598 | HLA-A*32:01 |
| 2045 | AFP | ENSG00000081051 | AVSVILRVA | 1598 | HLA-B*56:01 |
| 2046 | AFP | ENSG00000081051 | AVSVILRV | 1599 | HLA-A*02:03 |
| 2047 | AFP | ENSG00000081051 | AVSVILRV | 1599 | HLA-B*13:02 |
| 2048 | AFP | ENSG00000081051 | AVSVILRV | 1599 | HLA-B*37:01 |
| 2049 | AFP | ENSG00000081051 | AYEEDRETFM | 1600 | HLA-C*04:01 |
| 2050 | AFP | ENSG00000081051 | AYEEDRETF | 1601 | HLA-A*23:01 |
| 2051 | AFP | ENSG00000081051 | AYEEDRETF | 1601 | HLA-A*24:02 |
| 2052 | AFP | ENSG00000081051 | AYEEDRETF | 1601 | HLA-B*38:01 |
| 2053 | AFP | ENSG00000081051 | AYEEDRETF | 1601 | HLA-B*55:01 |
| 2054 | AFP | ENSG00000081051 | AYEEDRETF | 1601 | HLA-C*04:01 |
| 2055 | AFP | ENSG00000081051 | AYEEDRETF | 1601 | HLA-C*14:02 |
| 2056 | AFP | ENSG00000081051 | AYTKKAPQL | 1602 | HLA-A*23:01 |
| 2057 | AFP | ENSG00000081051 | AYTKKAPQL | 1602 | HLA-A*24:02 |
| 2058 | AFP | ENSG00000081051 | AYTKKAPQL | 1602 | HLA-C*14:02 |
| 2059 | AFP | ENSG00000081051 | AYTKKAPQL | 1602 | HLA-C*16:01 |
| 2060 | AFP | ENSG00000081051 | CFQTKAATV | 1603 | HLA-C*14:02 |
| 2061 | AFP | ENSG00000081051 | CLQDGEKIMSY | 1604 | HLA-A*01:01 |
| 2062 | AFP | ENSG00000081051 | CLQDGEKIMSY | 1604 | HLA-A*03:01 |
| 2063 | AFP | ENSG00000081051 | CLQDGEKIMSY | 1604 | HLA-A*03:02 |
| 2064 | AFP | ENSG00000081051 | CLQDGEKIMSY | 1604 | HLA-A*25:01 |
| 2065 | AFP | ENSG00000081051 | CLQDGEKIMSY | 1604 | HLA-A*26:01 |
| 2066 | AFP | ENSG00000081051 | CLQDGEKIMSY | 1604 | HLA-A*30:02 |
| 2067 | AFP | ENSG00000081051 | CLQDGEKIMSY | 1604 | HLA-A*32:01 |
| 2068 | AFP | ENSG00000081051 | CLQDGEKIMSY | 1604 | HLA-B*15:01 |
| 2069 | AFP | ENSG00000081051 | CQAQGVALQTM | 1605 | HLA-C*07:04 |
| 2070 | AFP | ENSG00000081051 | CQAQGVAL | 1606 | HLA-B*15:01 |
| 2071 | AFP | ENSG00000081051 | CQAQGVAL | 1606 | HLA-B*27:05 |
| 2072 | AFP | ENSG00000081051 | CQAQGVAL | 1606 | HLA-B*39:01 |
| 2073 | AFP | ENSG00000081051 | CQAQGVAL | 1606 | HLA-C*03:04 |
| 2074 | AFP | ENSG00000081051 | CQDKGEEEL | 1607 | HLA-B*38:01 |
| 2075 | AFP | ENSG00000081051 | CQDKGEEEL | 1607 | HLA-B*39:01 |
| 2076 | AFP | ENSG00000081051 | CSQQDTLSNK | 1608 | HLA-A*03:02 |
| 2077 | AFP | ENSG00000081051 | CSQQDTLSNK | 1608 | HLA-A*11:01 |
| 2078 | AFP | ENSG00000081051 | CSQQDTLSNK | 1608 | HLA-B*27:02 |
| 2079 | AFP | ENSG00000081051 | DALTAMKP | 1609 | HLA-A*33:01 |
| 2080 | AFP | ENSG00000081051 | DALTMEK | 1610 | HLA-A*33:01 |
| 2081 | AFP | ENSG00000081051 | DALTALEK | 1610 | HLA-B*51:01 |
| 2082 | AFP | ENSG00000081051 | DEKPEGLSP | 1611 | HLA-B*18:01 |
| 2083 | AFP | ENSG00000081051 | DEKPEGLSP | 1611 | HLA-B*40:02 |
| 2084 | AFP | ENSG00000081051 | DETYVPPAF | 1612 | HLA-A*23:01 |
| 2085 | AFP | ENSG00000081051 | DETYVPPAF | 1612 | HLA-A*25:01 |
| 2086 | AFP | ENSG00000081051 | DETYVPPAF | 1612 | HLA-A*30:01 |
| 2087 | AFP | ENSG00000081051 | DETYVPPAF | 1612 | HLA-B*18:01 |
| 2088 | AFP | ENSG00000081051 | DETYVPPAF | 1612 | HLA-B*27:02 |
| 2089 | AFP | ENSG00000081051 | DETYVPPAF | 1612 | HLA-B*35:01 |
| 2090 | AFP | ENSG00000081051 | DETYVPPAF | 1612 | HLA-B*40:01 |
| 2091 | AFP | ENSG00000081051 | DETYVPPAF | 1612 | HLA-B*44:02 |
| 2092 | AFP | ENSG00000081051 | DETYVPPAF | 1612 | HLA-B*44:03 |
| 2093 | AFP | ENSG00000081051 | DETYVPPAF | 1612 | HLA-C*16:04 |
| 2094 | AFP | ENSG00000081051 | DETYVPPA | 1613 | HLA-B*18:01 |
| 2095 | AFP | ENSG00000081051 | DFNQFSSGEK | 1614 | HLA-A*33:01 |
| 2096 | AFP | ENSG00000081051 | DGEKIMSYI | 1615 | HLA-B*51:01 |
| 2097 | AFP | ENSG00000081051 | DGEKIMSY | 1616 | HLA-B*18:01 |
| 2098 | AFP | ENSG00000081051 | DGEKIMSY | 1616 | HLA-C*07:01 |
| 2099 | AFP | ENSG00000081051 | DLATIFFAQFV | 1617 | HLA-A*68:02 |
| 2100 | AFP | ENSG00000081051 | DLATIFFAQF | 1618 | HLA-A*25:01 |
| 2101 | AFP | ENSG00000081051 | DLATIFFAQF | 1618 | HLA-A*26:01 |
| 2102 | AFP | ENSG00000081051 | DSYQCTAEI | 1619 | HLA-B*51:01 |
| 2103 | AFP | ENSG00000081051 | DTLSNKITE | 1620 | HLA-A*33:01 |
| 2104 | AFP | ENSG00000081051 | EATYKEVSK | 1621 | HLA-A*33:01 |
| 2105 | AFP | ENSG00000081051 | EATYKEVSK | 1621 | HLA-A*33:03 |
| 2106 | AFP | ENSG00000081051 | EATYKEVSK | 1621 | HLA-A*68:01 |
| 2107 | AFP | ENSG00000081051 | EATYKEVSK | 1621 | HLA-C*07:06 |
| 2108 | AFP | ENSG00000081051 | EAVIADFSGL | 1622 | HLA-A*25:01 |
| 2109 | AFP | ENSG00000081051 | EAVIADFSGL | 1622 | HLA-A*26:01 |
| 2110 | AFP | ENSG00000081051 | EAYEEDRETFM | 1623 | HLA-A*26:01 |
| 2111 | AFP | ENSG00000081051 | EAYEEDRETF | 1624 | HLA-A*25:01 |
| 2112 | AFP | ENSG00000081051 | EAYEEDRETF | 1624 | HLA-A*26:01 |
| 2113 | AFP | ENSG00000081051 | EAYEEDRETF | 1624 | HLA-A*33:03 |
| 2114 | AFP | ENSG00000081051 | EAYEEDRETF | 1624 | HLA-B*15:01 |
| 2115 | AFP | ENSG00000081051 | EAYEEDRETF | 1624 | HLA-B*15:03 |
| 2116 | AFP | ENSG00000081051 | EAYEEDRETF | 1624 | HLA-B*27:02 |
| 2117 | AFP | ENSG00000081051 | EAYEEDRETF | 1624 | HLA-B*35:01 |
| 2118 | AFP | ENSG00000081051 | EAYEEDRETF | 1624 | HLA-B*35:03 |
| 2119 | AFP | ENSG00000081051 | EAYEEDRETF | 1624 | HLA-B*38:01 |
| 2120 | AFP | ENSG00000081051 | EAYEEDRETF | 1624 | HLA-B*46:01 |
| 2121 | AFP | ENSG00000081051 | EAYEEDRETF | 1624 | HLA-B*51:01 |
| 2122 | AFP | ENSG00000081051 | EAYEEDRETF | 1624 | HLA-B*55:01 |
| 2123 | AFP | ENSG00000081051 | EAYEEDRETF | 1624 | HLA-B*57:01 |
| 2124 | AFP | ENSG00000081051 | EAYEEDRETF | 1624 | HLA-B*58:01 |
| 2125 | AFP | ENSG00000081051 | EAYEEDRETF | 1624 | HLA-C*02:02 |
| 2126 | AFP | ENSG00000081051 | EAYEEDRETF | 1624 | HLA-C*03:03 |
| 2127 | AFP | ENSG00000081051 | EAYEEDRETF | 1624 | HLA-C*05:01 |
| 2128 | AFP | ENSG00000081051 | EAYEEDRETF | 1624 | HLA-C*07:06 |
| 2129 | AFP | ENSG00000081051 | EAYEEDRETF | 1624 | HLA-C*16:04 |
| 2130 | AFP | ENSG00000081051 | EEDRETFMNKF | 1625 | HLA-B*44:02 |
| 2131 | AFP | ENSG00000081051 | EEDRETFMNKF | 1625 | HLA-B*44:03 |
| 2132 | AFP | ENSG00000081051 | EEGQKLISK | 1626 | HLA-B*44:02 |
| 2133 | AFP | ENSG00000081051 | EEGQKLISK | 1626 | HLA-B*44:03 |
| 2134 | AFP | ENSG00000081051 | EEQLEAVIADF | 1627 | HLA-B*44:02 |
| 2135 | AFP | ENSG00000081051 | EEQLEAVIADF | 1627 | HLA-B*44:03 |
| 2136 | AFP | ENSG00000081051 | EGAADIIIGHL | 1628 | HLA-A*68:02 |
| 2137 | AFP | ENSG00000081051 | EGAADIIIGH | 1629 | HLA-A*68:01 |
| 2138 | AFP | ENSG00000081051 | EGAADIIIGH | 1629 | HLA-C*07:06 |
| 2139 | AFP | ENSG00000081051 | EGLSPNLNR | 1630 | HLA-A*33:01 |
| 2140 | AFP | ENSG00000081051 | EGLSPNLNR | 1630 | HLA-A*33:03 |
| 2141 | AFP | ENSG00000081051 | EGLSPNLNR | 1630 | HLA-A*68:01 |
| 2142 | AFP | ENSG00000081051 | EGLSPNLNR | 1630 | HLA-C*07:06 |
| 2143 | AFP | ENSG00000081051 | EGQKLISKTR | 1631 | HLA-A*33:03 |
| 2144 | AFP | ENSG00000081051 | EIQKLVLDV | 1632 | HLA-A*68:02 |
| 2145 | AFP | ENSG00000081051 | ELMAITRKMAA | 1633 | HLA-B*08:01 |
| 2146 | AFP | ENSG00000081051 | ELMAITRKM | 1634 | HLA-A*25:01 |
| 2147 | AFP | ENSG00000081051 | ELMAITRKM | 1634 | HLA-A*26:01 |
| 2148 | AFP | ENSG00000081051 | ELMAITRKM | 1634 | HLA-A*33:03 |
| 2149 | AFP | ENSG00000081051 | ELMAITRKM | 1634 | HLA-B*08:01 |
| 2150 | AFP | ENSG00000081051 | ELMAITRKM | 1634 | HLA-B*44:02 |
| 2151 | AFP | ENSG00000081051 | ELMAITRKM | 1634 | HLA-B*44:03 |
| 2152 | AFP | ENSG00000081051 | ELRESSLLNQH | 1635 | HLA-A*33:01 |
| 2153 | AFP | ENSG00000081051 | EMTPVNPGV | 1636 | HLA-A*02:01 |
| 2154 | AFP | ENSG00000081051 | EMTPVNPGV | 1636 | HLA-A*02:03 |
| 2155 | AFP | ENSG00000081051 | EMTPVNPGV | 1636 | HLA-A*02:07 |
| 2156 | AFP | ENSG00000081051 | EMTPVNPGV | 1636 | HLA-A*26:01 |
| 2157 | AFP | ENSG00000081051 | EMTPVNPGV | 1636 | HLA-A*30:01 |
| 2158 | AFP | ENSG00000081051 | EMTPVNPGV | 1636 | HLA-A*68:02 |
| 2159 | AFP | ENSG00000081051 | EMTPVNPGV | 1636 | HLA-B*13:02 |
| 2160 | AFP | ENSG00000081051 | EMTPVNPGV | 1636 | HLA-B*27:05 |
| 2161 | AFP | ENSG00000081051 | EMTPVNPGV | 1636 | HLA-B*55:01 |
| 2162 | AFP | ENSG00000081051 | EMTPVNPGV | 1636 | HLA-C*04:01 |
| 2163 | AFP | ENSG00000081051 | EMTPVNPGV | 1636 | HLA-C*06:02 |
| 2164 | AFP | ENSG00000081051 | EMTPVNPGV | 1636 | HLA-C*16:02 |
| 2165 | AFP | ENSG00000081051 | ENDEKPEGL | 1637 | HLA-C*05:01 |
| 2166 | AFP | ENSG00000081051 | EPVTSCEAY | 1638 | HLA-A*26:01 |
| 2167 | AFP | ENSG00000081051 | EPVTSCEAY | 1638 | HLA-B*35:01 |
| 2168 | AFP | ENSG00000081051 | EPVTSCEAY | 1638 | HLA-B*55:01 |
| 2169 | AFP | ENSG00000081051 | EQLEAVIADF | 1639 | HLA-A*25:01 |
| 2170 | AFP | ENSG00000081051 | EQLEAVIADF | 1639 | HLA-A*26:01 |
| 2171 | AFP | ENSG00000081051 | ESIFLIFLL | 1640 | HLA-A*68:02 |
| 2172 | AFP | ENSG00000081051 | ESQALAKR | 1641 | HLA-A*33:01 |
| 2173 | AFP | ENSG00000081051 | ESQALAKR | 1641 | HLA-A*33:03 |
| 2174 | AFP | ENSG00000081051 | ETFMNKFIY | 1642 | HLA-A*01:01 |
| 2175 | AFP | ENSG00000081051 | ETFMNKFIY | 1642 | HLA-A*26:01 |
| 2176 | AFP | ENSG00000081051 | ETYVPPAF | 1643 | HLA-A*25:01 |
| 2177 | AFP | ENSG00000081051 | ETYVPPAF | 1643 | HLA-A*26:01 |
| 2178 | AFP | ENSG00000081051 | ETYVPPAF | 1643 | HLA-B*18:01 |
| 2179 | AFP | ENSG00000081051 | ETYVPPAF | 1643 | HLA-B*57:01 |
| 2180 | AFP | ENSG00000081051 | ETYVPPAF | 1643 | HLA-C*05:01 |
| 2181 | AFP | ENSG00000081051 | EVSKMVKDAL | 1644 | HLA-A*68:01 |
| 2182 | AFP | ENSG00000081051 | EVSKMVKDAL | 1644 | HLA-A*68:02 |
| 2183 | AFP | ENSG00000081051 | EVSKMVKDAL | 1644 | HLA-B*07:02 |
| 2184 | AFP | ENSG00000081051 | EVSKMVKDAL | 1644 | HLA-C*07:01 |
| 2185 | AFP | ENSG00000081051 | EYGIASILDSY | 1645 | HLA-A*29:02 |
| 2186 | AFP | ENSG00000081051 | EYGIASILDSY | 1645 | HLA-A*30:02 |
| 2187 | AFP | ENSG00000081051 | EYGIASIL | 1646 | HLA-C*14:02 |
| 2188 | AFP | ENSG00000081051 | EYSRRHPQL | 1647 | HLA-A*24:02 |
| 2189 | AFP | ENSG00000081051 | EYYLQNAFL | 1648 | HLA-A*23:01 |
| 2190 | AFP | ENSG00000081051 | EYYLQNAFL | 1648 | HLA-A*24:02 |
| 2191 | AFP | ENSG00000081051 | EYYLQNAF | 1649 | HLA-C*14:02 |
| 2192 | AFP | ENSG00000081051 | FAEEGQKLISK | 1650 | HLA-A*01:01 |
| 2193 | AFP | ENSG00000081051 | FAEEGQKLISK | 1650 | HLA-B*27:02 |
| 2194 | AFP | ENSG00000081051 | FAEEGQKLI | 1651 | HLA-B*38:01 |
| 2195 | AFP | ENSG00000081051 | FAEEGQKLI | 1651 | HLA-B*49:01 |
| 2196 | AFP | ENSG00000081051 | FAEEGQKLI | 1651 | HLA-B*51:01 |
| 2197 | AFP | ENSG00000081051 | FAEEGQKLI | 1651 | HLA-C*03:03 |
| 2198 | AFP | ENSG00000081051 | FAEEGQKLI | 1651 | HLA-C*03:04 |
| 2199 | AFP | ENSG00000081051 | FAEEGQKLI | 1651 | HLA-C*05:01 |
| 2200 | AFP | ENSG00000081051 | FAEEGQKLI | 1651 | HLA-C*16:02 |
| 2201 | AFP | ENSG00000081051 | FAEEGQKL | 1652 | HLA-B*35:03 |
| 2202 | AFP | ENSG00000081051 | FAEEGQKL | 1652 | HLA-B*39:01 |
| 2203 | AFP | ENSG00000081051 | FAEEGQKL | 1652 | HLA-C*01:02 |
| 2204 | AFP | ENSG00000081051 | FAEEGQKL | 1652 | HLA-C*03:03 |
| 2205 | AFP | ENSG00000081051 | FAEEGQKL | 1652 | HLA-C*03:04 |
| 2206 | AFP | ENSG00000081051 | FAEEGQKL | 1652 | HLA-C*05:01 |
| 2207 | AFP | ENSG00000081051 | FAQFVQEATYK | 1653 | HLA-B*27:02 |
| 2208 | AFP | ENSG00000081051 | FAQFVQEATY | 1654 | HLA-A*01:01 |
| 2209 | AFP | ENSG00000081051 | FAQFVQEATY | 1654 | HLA-A*29:02 |
| 2210 | AFP | ENSG00000081051 | FAQFVQEATY | 1654 | HLA-A*30:02 |
| 2211 | AFP | ENSG00000081051 | FAQFVQEATY | 1654 | HLA-B*27:05 |
| 2212 | AFP | ENSG00000081051 | FAQFVQEATY | 1654 | HLA-B*35:01 |
| 2213 | AFP | ENSG00000081051 | FAQFVQEATY | 1654 | HLA-B*39:01 |
| 2214 | AFP | ENSG00000081051 | FAQFVQEATY | 1654 | HLA-B*46:01 |
| 2215 | AFP | ENSG00000081051 | FAQFVQEATY | 1654 | HLA-B*55:01 |
| 2216 | AFP | ENSG00000081051 | FAQFVQEATY | 1654 | HLA-C*02:02 |
| 2217 | AFP | ENSG00000081051 | FAQFVQEATY | 1654 | HLA-C*03:03 |
| 2218 | AFP | ENSG00000081051 | FAQFVQEATY | 1654 | HLA-C*04:01 |
| 2219 | AFP | ENSG00000081051 | FAQFVQEATY | 1654 | HLA-C*07:01 |
| 2220 | AFP | ENSG00000081051 | FAQFVQEATY | 1654 | HLA-C*12:03 |
| 2221 | AFP | ENSG00000081051 | FAQFVQEATY | 1654 | HLA-C*14:02 |
| 2222 | AFP | ENSG00000081051 | FAQFVQEATY | 1654 | HLA-C*16:02 |
| 2223 | AFP | ENSG00000081051 | FAQFVQEATY | 1654 | HLA-C*16:04 |
| 2224 | AFP | ENSG00000081051 | FAQFVQEA | 1655 | HLA-B*54:01 |
| 2225 | AFP | ENSG00000081051 | FFAQFVQEATY | 1656 | HLA-A*29:02 |
| 2226 | AFP | ENSG00000081051 | FFAQFVQEA | 1657 | HLA-C*14:02 |
| 2227 | AFP | ENSG00000081051 | FIFHKDLCQA | 1658 | HLA-A*02:03 |
| 2228 | AFP | ENSG00000081051 | FLAHKKPTPA | 1659 | HLA-A*02:03 |
| 2229 | AFP | ENSG00000081051 | FLASFVHEY | 1660 | HLA-A*01:01 |
| 2230 | AFP | ENSG00000081051 | FLASFVHEY | 1660 | HLA-A*02:01 |
| 2231 | AFP | ENSG00000081051 | FLASFVHEY | 1660 | HLA-A*02:03 |
| 2232 | AFP | ENSG00000081051 | FLASFVHEY | 1660 | HLA-A*02:04 |
| 2233 | AFP | ENSG00000081051 | FLASFVHEY | 1660 | HLA-A*02:07 |
| 2234 | AFP | ENSG00000081051 | FLASFVHEY | 1660 | HLA-A*03:01 |
| 2235 | AFP | ENSG00000081051 | FLASFVHEY | 1660 | HLA-A*25:01 |
| 2236 | AFP | ENSG00000081051 | FLASFVHEY | 1660 | HLA-A*26:01 |
| 2237 | AFP | ENSG00000081051 | FLASFVHEY | 1660 | HLA-A*29:02 |
| 2238 | AFP | ENSG00000081051 | FLASFVHEY | 1660 | HLA-A*30:02 |
| 2239 | AFP | ENSG00000081051 | FLASFVHEY | 1660 | HLA-A*32:01 |
| 2240 | AFP | ENSG00000081051 | FLASFVHEY | 1660 | HLA-A*33:01 |
| 2241 | AFP | ENSG00000081051 | FLASFVHEY | 1660 | HLA-A*68:02 |
| 2242 | AFP | ENSG00000081051 | FLASFVHEY | 1660 | HLA-B*15:01 |
| 2243 | AFP | ENSG00000081051 | FLASFVHEY | 1660 | HLA-B*15:03 |
| 2244 | AFP | ENSG00000081051 | FLASFVHEY | 1660 | HLA-B*18:01 |
| 2245 | AFP | ENSG00000081051 | FLASFVHEY | 1660 | HLA-B*35:01 |
| 2246 | AFP | ENSG00000081051 | FLASFVHEY | 1660 | HLA-B*44:02 |
| 2247 | AFP | ENSG00000081051 | FLASFVHEY | 1660 | HLA-B*44:03 |
| 2248 | AFP | ENSG00000081051 | FLASFVHEY | 1660 | HLA-B*46:01 |
| 2249 | AFP | ENSG00000081051 | FLASFVHEY | 1660 | HLA-B*57:01 |
| 2250 | AFP | ENSG00000081051 | FLASFVHEY | 1660 | HLA-C*02:02 |
| 2251 | AFP | ENSG00000081051 | FLASFVHEY | 1660 | HLA-C*07:04 |
| 2252 | AFP | ENSG00000081051 | FLASFVHEY | 1660 | HLA-C*12:03 |
| 2253 | AFP | ENSG00000081051 | FLASFVHEY | 1660 | HLA-C*16:01 |
| 2254 | AFP | ENSG00000081051 | FLASFVHEY | 1660 | HLA-C*16:02 |
| 2255 | AFP | ENSG00000081051 | FLASFVHEY | 1660 | HLA-C*16:04 |
| 2256 | AFP | ENSG00000081051 | FLGDRDFNQF | 1661 | HLA-A*24:02 |
| 2257 | AFP | ENSG00000081051 | FLLNFTESRTL | 1662 | HLA-A*02:01 |
| 2258 | AFP | ENSG00000081051 | FLLNFTESRTL | 1662 | HLA-A*02:03 |
| 2259 | AFP | ENSG00000081051 | FLLNFTESRTL | 1662 | HLA-A*02:04 |
| 2260 | AFP | ENSG00000081051 | FLLNFTESR | 1663 | HLA-A*33:01 |
| 2261 | AFP | ENSG00000081051 | FLLNFTESR | 1663 | HLA-A*33:03 |
| 2262 | AFP | ENSG00000081051 | FLVAYTKKA | 1664 | HLA-A*02:03 |
| 2263 | AFP | ENSG00000081051 | FLYAPTILLW | 1665 | HLA-A*29:02 |
| 2264 | AFP | ENSG00000081051 | FLYAPTILL | 1666 | HLA-A*02:01 |
| 2265 | AFP | ENSG00000081051 | FLYAPTILL | 1666 | HLA-A*02:03 |
| 2266 | AFP | ENSG00000081051 | FLYAPTILL | 1666 | HLA-A*02:04 |
| 2267 | AFP | ENSG00000081051 | FLYAPTILL | 1666 | HLA-A*02:07 |
| 2268 | AFP | ENSG00000081051 | FMNKFIYEI | 1667 | HLA-A*02:01 |
| 2269 | AFP | ENSG00000081051 | FMNKFIYEI | 1667 | HLA-A*02:03 |
| 2270 | AFP | ENSG00000081051 | FMNKFIYEI | 1667 | HLA-A*02:04 |
| 2271 | AFP | ENSG00000081051 | FMNKFIYEI | 1667 | HLA-A*02:07 |
| 2272 | AFP | ENSG00000081051 | FQAITVTKL | 1668 | HLA-A*02:01 |
| 2273 | AFP | ENSG00000081051 | FQAITVTKL | 1668 | HLA-A*02:03 |
| 2274 | AFP | ENSG00000081051 | FQAITVTKL | 1668 | HLA-A*02:04 |
| 2275 | AFP | ENSG00000081051 | FQAITVTKL | 1668 | HLA-A*24:02 |
| 2276 | AFP | ENSG00000081051 | FQAITVTKL | 1668 | HLA-A*30:01 |
| 2277 | AFP | ENSG00000081051 | FQAITVTKL | 1668 | HLA-A*32:01 |
| 2278 | AFP | ENSG00000081051 | FQAITVTKL | 1668 | HLA-A*68:02 |
| 2279 | AFP | ENSG00000081051 | FQAITVTKL | 1668 | HLA-B*13:02 |
| 2280 | AFP | ENSG00000081051 | FQAITVTKL | 1668 | HLA-B*15:01 |
| 2281 | AFP | ENSG00000081051 | FQAITVTKL | 1668 | HLA-B*I5:03 |
| 2282 | AFP | ENSG00000081051 | FQAITVTKL | 1668 | HLA-B*27:05 |
| 2283 | AFP | ENSG00000081051 | FQAITVTKL | 1668 | HLA-B*38:01 |
| 2284 | AFP | ENSG00000081051 | FQAITVTKL | 1668 | HLA-B*39:01 |
| 2285 | AFP | ENSG00000081051 | FQAITVTKL | 1668 | HLA-B*40:01 |
| 2286 | AFP | ENSG00000081051 | FQAITVTKL | 1668 | HLA-B*40:02 |
| 2287 | AFP | ENSG00000081051 | FQAITVTKL | 1668 | HLA-B*58:01 |
| 2288 | AFP | ENSG00000081051 | FQAITVTKL | 1668 | HLA-C*02:02 |
| 2289 | AFP | ENSG00000081051 | FQAITVTKL | 1668 | HLA-C*06:02 |
| 2290 | AFP | ENSG00000081051 | FQAITVTKL | 1668 | HLA-C*07:04 |
| 2291 | AFP | ENSG00000081051 | FQAITVTKL | 1668 | HLA-C*12:03 |
| 2292 | AFP | ENSG00000081051 | FQKLGEYY | 1669 | HLA-A*30:02 |
| 2293 | AFP | ENSG00000081051 | FQKLGEYY | 1669 | HLA-B*15:01 |
| 2294 | AFP | ENSG00000081051 | FQKLGEYY | 1669 | HLA-B*15:03 |
| 2295 | AFP | ENSG00000081051 | FQKLGEYY | 1669 | HLA-B*46:01 |
| 2296 | AFP | ENSG00000081051 | FQKLGEYY | 1669 | HLA-C*02:02 |
| 2297 | AFP | ENSG00000081051 | FQKLGEYY | 1669 | HLA-C*07:04 |
| 2298 | AFP | ENSG00000081051 | FQTENPLEC | 1670 | HLA-A*02:01 |
| 2299 | AFP | ENSG00000081051 | FQTENPLEC | 1670 | HLA-A*30:01 |
| 2300 | AFP | ENSG00000081051 | FQTENPLEC | 1670 | HLA-B*13:02 |
| 2301 | AFP | ENSG00000081051 | FQTENPLEC | 1670 | HLA-B*39:01 |
| 2302 | AFP | ENSG00000081051 | FQTENPLEC | 1670 | HLA-C*02:02 |
| 2303 | AFP | ENSG00000081051 | FQTENPLEC | 1670 | HLA-C*03:03 |
| 2304 | AFP | ENSG00000081051 | FQTENPLEC | 1670 | HLA-C*03:04 |
| 2305 | AFP | ENSG00000081051 | FQTENPLEC | 1670 | HLA-C*12:03 |
| 2306 | AFP | ENSG00000081051 | FQTENPLEC | 1670 | HLA-C*16:02 |
| 2307 | AFP | ENSG00000081051 | FQTKAATVTK | 1671 | HLA-B*27:05 |
| 2308 | AFP | ENSG00000081051 | FQTKAATVT | 1672 | HLA-B*13:02 |
| 2309 | AFP | ENSG00000081051 | FQTKAATV | 1673 | HLA-B*13:02 |
| 2310 | AFP | ENSG00000081051 | FQVPEPVTS | 1674 | HLA-B*27:05 |
| 2311 | AFP | ENSG00000081051 | FSDDKFIFH | 1675 | HLA-A*01:01 |
| 2312 | AFP | ENSG00000081051 | FSSGEKNIF | 1676 | HLA-C*16:01 |
| 2313 | AFP | ENSG00000081051 | FSSLVVDETY | 1677 | HLA-A*01:01 |
| 2314 | AFP | ENSG00000081051 | FTEIQKLVL | 1678 | HLA-A*01:01 |
| 2315 | AFP | ENSG00000081051 | FTEIQKLVL | 1678 | HLA-C*03:03 |
| 2316 | AFP | ENSG00000081051 | FTEIQKLVL | 1678 | HLA-C*03:04 |
| 2317 | AFP | ENSG00000081051 | FTESRTLHR | 1679 | HLA-A*01:01 |
| 2318 | AFP | ENSG00000081051 | FTKVNFTEI | 1680 | HLA-A*02:03 |
| 2319 | AFP | ENSG00000081051 | FTKVNFTEI | 1680 | HLA-A*24:02 |
| 2320 | AFP | ENSG00000081051 | FTKVNFTEI | 1680 | HLA-A*25:01 |
| 2321 | AFP | ENSG00000081051 | FTKVNFTEI | 1680 | HLA-A*68:02 |
| 2322 | AFP | ENSG00000081051 | FTKVNFTEI | 1680 | HLA-B*40:02 |
| 2323 | AFP | ENSG00000081051 | FTKVNFTEI | 1680 | HLA-B*46:01 |
| 2324 | AFP | ENSG00000081051 | FTKVNFTEI | 1680 | HLA-C*02:02 |
| 2325 | AFP | ENSG00000081051 | FTKVNFTEI | 1680 | HLA-C*03:04 |
| 2326 | AFP | ENSG00000081051 | FVQEATYKEV | 1681 | HLA-A*02:03 |
| 2327 | AFP | ENSG00000081051 | FVQEATYKEV | 1681 | HLA-A*02:07 |
| 2328 | AFP | ENSG00000081051 | FVQEATYKEV | 1681 | HLA-A*25:01 |
| 2329 | AFP | ENSG00000081051 | FVQEATYKEV | 1681 | HLA-A*26:01 |
| 2330 | AFP | ENSG00000081051 | FVQEATYKEV | 1681 | HLA-A*68:02 |
| 2331 | AFP | ENSG00000081051 | FVQEATYKEV | 1681 | HLA-C*02:02 |
| 2332 | AFP | ENSG00000081051 | FVQEATYKEV | 1681 | HLA-C*03:04 |
| 2333 | AFP | ENSG00000081051 | FVQEATYK | 1682 | HLA-A*03:02 |
| 2334 | AFP | ENSG00000081051 | FVQEATYK | 1682 | HLA-A*11:01 |
| 2335 | AFP | ENSG00000081051 | FVQEATYK | 1682 | HLA-B*27:02 |
| 2336 | AFP | ENSG00000081051 | FVQEATYK | 1682 | HLA-C*04:01 |
| 2337 | AFP | ENSG00000081051 | GAADIIIGHL | 1683 | HLA-A*02:03 |
| 2338 | AFP | ENSG00000081051 | GAADIIIGHL | 1683 | HLA-A*02:04 |
| 2339 | AFP | ENSG00000081051 | GAADIIIGHL | 1683 | HLA-A*25:01 |
| 2340 | AFP | ENSG00000081051 | GAADIIIGHL | 1683 | HLA-A*26:01 |
| 2341 | AFP | ENSG00000081051 | GAADIIIGHL | 1683 | HLA-A*30:01 |
| 2342 | AFP | ENSG00000081051 | GAADIIIGHL | 1683 | HLA-A*68:02 |
| 2343 | AFP | ENSG00000081051 | GAADIIIGH | 1684 | HLA-A*03:01 |
| 2344 | AFP | ENSG00000081051 | GAADIIIGH | 1684 | HLA-A*03:02 |
| 2345 | AFP | ENSG00000081051 | GAADIIIGH | 1684 | HLA-A*II:01 |
| 2346 | AFP | ENSG00000081051 | GAADIIIGH | 1684 | HLA-A*26:01 |
| 2347 | AFP | ENSG00000081051 | GAADIIIGH | 1684 | HLA-A*68:01 |
| 2348 | AFP | ENSG00000081051 | GAADIIIGH | 1684 | HLA-B*27:05 |
| 2349 | AFP | ENSG00000081051 | GAADIIIGH | 1684 | HLA-C*02:02 |
| 2350 | AFP | ENSG00000081051 | GAADIIIGH | 1684 | HLA-C*07:06 |
| 2351 | AFP | ENSG00000081051 | GAADIIIGH | 1684 | HLA-C*12:03 |
| 2352 | AFP | ENSG00000081051 | GAADIIIGH | 1684 | HLA-C*16:04 |
| 2353 | AFP | ENSG00000081051 | GDRDFNQF | 1685 | HLA-B*18:01 |
| 2354 | AFP | ENSG00000081051 | GDRDFNQF | 1685 | HLA-B*37:01 |
| 2355 | AFP | ENSG00000081051 | GEEELQKYI | 1686 | HLA-B*40:01 |
| 2356 | AFP | ENSG00000081051 | GEEELQKYI | 1686 | HLA-B*44:02 |
| 2357 | AFP | ENSG00000081051 | GEEELQKYI | 1686 | HLA-B*44:03 |
| 2358 | AFP | ENSG00000081051 | GEEELQKYI | 1686 | HLA-B*49:01 |
| 2359 | AFP | ENSG00000081051 | GEEELQKY | 1687 | HLA-B*18:01 |
| 2360 | AFP | ENSG00000081051 | GEEELQKY | 1687 | HLA-B*44:02 |
| 2361 | AFP | ENSG00000081051 | GEGAADIII | 1688 | HLA-B*40:01 |
| 2362 | AFP | ENSG00000081051 | GEGAADIII | 1688 | HLA-B*49:01 |
| 2363 | AFP | ENSG00000081051 | GEKIMSYI | 1689 | HLA-B*49:01 |
| 2364 | AFP | ENSG00000081051 | GEKNIFLASF | 1690 | HLA-B*44:02 |
| 2365 | AFP | ENSG00000081051 | GEYYLQNAFL | 1691 | HLA-B*40:01 |
| 2366 | AFP | ENSG00000081051 | GEYYLQNAFL | 1691 | HLA-B*49:01 |
| 2367 | AFP | ENSG00000081051 | GEYYLQNAF | 1692 | HLA-A*30:01 |
| 2368 | AFP | ENSG00000081051 | GEYYLQNAF | 1692 | HLA-B*13:02 |
| 2369 | AFP | ENSG00000081051 | GEYYLQNAF | 1692 | HLA-B*15:01 |
| 2370 | AFP | ENSG00000081051 | GEYYLQNAF | 1692 | HLA-B*15:03 |
| 2371 | AFP | ENSG00000081051 | GEYYLQNAF | 1692 | HLA-B*18:01 |
| 2372 | AFP | ENSG00000081051 | GEYYLQNAF | 1692 | HLA-B*27:02 |
| 2373 | AFP | ENSG00000081051 | GEYYLQNAF | 1692 | HLA-B*37:01 |
| 2374 | AFP | ENSG00000081051 | GEYYLQNAF | 1692 | HLA-B*40:01 |
| 2375 | AFP | ENSG00000081051 | GEYYLQNAF | 1692 | HLA-B*44:02 |
| 2376 | AFP | ENSG00000081051 | GEYYLQNAF | 1692 | HLA-B*44:03 |
| 2377 | AFP | ENSG00000081051 | GEYYLQNAF | 1692 | HLA-B*49:01 |
| 2378 | AFP | ENSG00000081051 | GEYYLQNAF | 1692 | HLA-C*16:04 |
| 2379 | AFP | ENSG00000081051 | GEYYLQNA | 1693 | HLA-B*49:01 |
| 2380 | AFP | ENSG00000081051 | GIASILDSY | 1694 | HLA-A*01:01 |
| 2381 | AFP | ENSG00000081051 | GIASILDSY | 1694 | HLA-A*25:01 |
| 2382 | AFP | ENSG00000081051 | GIASILDSY | 1694 | HLA-A*26:01 |
| 2383 | AFP | ENSG00000081051 | GIASILDSY | 1694 | HLA-A*29:02 |
| 2384 | AFP | ENSG00000081051 | GIASILDSY | 1694 | HLA-A*30:02 |
| 2385 | AFP | ENSG00000081051 | GIASILDSY | 1694 | HLA-A*32:01 |
| 2386 | AFP | ENSG00000081051 | GIASILDSY | 1694 | HLA-B*15:01 |
| 2387 | AFP | ENSG00000081051 | GIASILDSY | 1694 | HLA-B*15:03 |
| 2388 | AFP | ENSG00000081051 | GIASILDSY | 1694 | HLA-B*27:05 |
| 2389 | AFP | ENSG00000081051 | GIASILDSY | 1694 | HLA-B*35:01 |
| 2390 | AFP | ENSG00000081051 | GIASILDSY | 1694 | HLA-B*44:03 |
| 2391 | AFP | ENSG00000081051 | GIASILDSY | 1694 | HLA-B*46:01 |
| 2392 | AFP | ENSG00000081051 | GIASILDSY | 1694 | HLA-B*55:01 |
| 2393 | AFP | ENSG00000081051 | GIASILDSY | 1694 | HLA-B*58:01 |
| 2394 | AFP | ENSG00000081051 | GIASILDSY | 1694 | HLA-C*02:02 |
| 2395 | AFP | ENSG00000081051 | GIASILDSY | 1694 | HLA-C*03:03 |
| 2396 | AFP | ENSG00000081051 | GIASILDSY | 1694 | HLA-C*07:04 |
| 2397 | AFP | ENSG00000081051 | GIASILDSY | 1694 | HLA-C*16:04 |
| 2398 | AFP | ENSG00000081051 | GLFQKLGEYYL | 1695 | HLA-A*02:01 |
| 2399 | AFP | ENSG00000081051 | GLFQKLGEYYL | 1695 | HLA-A*02:04 |
| 2400 | AFP | ENSG00000081051 | GLFQKLGEYY | 1696 | HLA-A*29:02 |
| 2401 | AFP | ENSG00000081051 | GLFQKLGEYY | 1696 | HLA-A*30:02 |
| 2402 | AFP | ENSG00000081051 | GLFQKLGEY | 1697 | HLA-A*03:01 |
| 2403 | AFP | ENSG00000081051 | GLFQKLGEY | 1697 | HLA-A*29:02 |
| 2404 | AFP | ENSG00000081051 | GLFQKLGEY | 1697 | HLA-A*30:02 |
| 2405 | AFP | ENSG00000081051 | GLFQKLGEY | 1697 | HLA-B*15:01 |
| 2406 | AFP | ENSG00000081051 | GLFQKLGEY | 1697 | HLA-B*15:03 |
| 2407 | AFP | ENSG00000081051 | GLFQKLGEY | 1697 | HLA-B*46:01 |
| 2408 | AFP | ENSG00000081051 | GLSPNLNRFL | 1698 | HLA-A*02:03 |
| 2409 | AFP | ENSG00000081051 | GLSPNLNRFL | 1698 | HLA-A*02:04 |
| 2410 | AFP | ENSG00000081051 | GLSPNLNRF | 1699 | HLA-B*15:01 |
| 2411 | AFP | ENSG00000081051 | GQKLISKTR | 1700 | HLA-A*31:01 |
| 2412 | AFP | ENSG00000081051 | GTRTFQAITV | 1701 | HLA-A*02:03 |
| 2413 | AFP | ENSG00000081051 | GTRTFQAITV | 1701 | HLA-C*06:02 |
| 2414 | AFP | ENSG00000081051 | HEKEILEKY | 1702 | HLA-A*29:02 |
| 2415 | AFP | ENSG00000081051 | HEKEILEKY | 1702 | HLA-A*30:01 |
| 2416 | AFP | ENSG00000081051 | HEKEILEKY | 1702 | HLA-A*30:02 |
| 2417 | AFP | ENSG00000081051 | HEKEILEKY | 1702 | HLA-B*15:03 |
| 2418 | AFP | ENSG00000081051 | HEKEILEKY | 1702 | HLA-B*18:01 |
| 2419 | AFP | ENSG00000081051 | HEKEILEKY | 1702 | HLA-B*44:02 |
| 2420 | AFP | ENSG00000081051 | HEKEILEKY | 1702 | HLA-B*44:03 |
| 2421 | AFP | ENSG00000081051 | HEKEILEKY | 1702 | HLA-C*02:02 |
| 2422 | AFP | ENSG00000081051 | HEKEILEKY | 1702 | HLA-C*16:04 |
| 2423 | AFP | ENSG00000081051 | HEMTPVNPGV | 1703 | HLA-A*30:01 |
| 2424 | AFP | ENSG00000081051 | HEMTPVNPGV | 1703 | HLA-B*40:01 |
| 2425 | AFP | ENSG00000081051 | HEMTPVNPGV | 1703 | HLA-B*49:01 |
| 2426 | AFP | ENSG00000081051 | HKKPTPASI | 1704 | HLA-B*15:03 |
| 2427 | AFP | ENSG00000081051 | HPFLYAPTI | 1705 | HLA-B*35:01 |
| 2428 | AFP | ENSG00000081051 | HPFLYAPTI | 1705 | HLA-B*35:03 |
| 2429 | AFP | ENSG00000081051 | HPFLYAPTI | 1705 | HLA-B*51:01 |
| 2430 | AFP | ENSG00000081051 | HPFLYAPTI | 1705 | HLA-B*54:01 |
| 2431 | AFP | ENSG00000081051 | HPFLYAPTI | 1705 | HLA-B*56:01 |
| 2432 | AFP | ENSG00000081051 | HPQLAVSVIL | 1706 | HLA-B*35:03 |
| 2433 | AFP | ENSG00000081051 | HPQLAVSVI | 1707 | HLA-B*07:02 |
| 2434 | AFP | ENSG00000081051 | HPQLAVSVI | 1707 | HLA-B*35:01 |
| 2435 | AFP | ENSG00000081051 | HPQLAVSVI | 1707 | HLA-B*35:03 |
| 2436 | AFP | ENSG00000081051 | HPQLAVSVI | 1707 | HLA-B*51:01 |
| 2437 | AFP | ENSG00000081051 | HPQLAVSVI | 1707 | HLA-B*54:01 |
| 2438 | AFP | ENSG00000081051 | HPQLAVSVI | 1707 | HLA-B*55:01 |
| 2439 | AFP | ENSG00000081051 | HPQLAVSVI | 1707 | HLA-B*56:01 |
| 2440 | AFP | ENSG00000081051 | HPQLAVSVI | 1707 | HLA-C*07:02 |
| 2441 | AFP | ENSG00000081051 | HPQLAVSV | 1708 | HLA-B*07:02 |
| 2442 | AFP | ENSG00000081051 | HPQLAVSV | 1708 | HLA-B*08:01 |
| 2443 | AFP | ENSG00000081051 | HPQLAVSV | 1708 | HLA-B*51:01 |
| 2444 | AFP | ENSG00000081051 | HPQLAVSV | 1708 | HLA-B*54:01 |
| 2445 | AFP | ENSG00000081051 | HPQLAVSV | 1708 | HLA-B*56:01 |
| 2446 | AFP | ENSG00000081051 | IADFSGLLEK | 1709 | HLA-A*01:01 |
| 2447 | AFP | ENSG00000081051 | IADFSGLLEK | 1709 | HLA-A*11:01 |
| 2448 | AFP | ENSG00000081051 | IADFSGLLEK | 1709 | HLA-B*27:02 |
| 2449 | AFP | ENSG00000081051 | IADFSGLL | 1710 | HLA-C*05:01 |
| 2450 | AFP | ENSG00000081051 | IASILDSY | 1711 | HLA-A*30:02 |
| 2451 | AFP | ENSG00000081051 | IASILDSY | 1711 | HLA-B*15:01 |
| 2452 | AFP | ENSG00000081051 | IASILDSY | 1711 | HLA-B*15:03 |
| 2453 | AFP | ENSG00000081051 | IASILDSY | 1711 | HLA-B*35:01 |
| 2454 | AFP | ENSG00000081051 | IASILDSY | 1711 | HLA-B*39:01 |
| 2455 | AFP | ENSG00000081051 | IASILDSY | 1711 | HLA-B*46:01 |
| 2456 | AFP | ENSG00000081051 | IASILDSY | 1711 | HLA-C*07:01 |
| 2457 | AFP | ENSG00000081051 | IASILDSY | 1711 | HLA-C*16:01 |
| 2458 | AFP | ENSG00000081051 | IASILDSY | 1711 | HLA-C*I6:02 |
| 2459 | AFP | ENSG00000081051 | ICSQQDTL | 1712 | HLA-B*39:01 |
| 2460 | AFP | ENSG00000081051 | IEKPTGDEQ | 1713 | HLA-B*40:02 |
| 2461 | AFP | ENSG00000081051 | IFLASFVHEY | 1714 | HLA-A*29:02 |
| 2462 | AFP | ENSG00000081051 | IFLASFVHEY | 1714 | HLA-A*30:02 |
| 2463 | AFP | ENSG00000081051 | IFLIFLLNF | 1715 | HLA-A*23:01 |
| 2464 | AFP | ENSG00000081051 | IFLIFLLNF | 1715 | HLA-A*24:02 |
| 2465 | AFP | ENSG00000081051 | IFLIFLLNF | 1715 | HLA-A*29:02 |
| 2466 | AFP | ENSG00000081051 | ILDSYQCTA | 1716 | HLA-A*02:01 |
| 2467 | AFP | ENSG00000081051 | ILDSYQCTA | 1716 | HLA-A*02:07 |
| 2468 | AFP | ENSG00000081051 | IPLFQVPEP | 1717 | HLA-B*54:01 |
| 2469 | AFP | ENSG00000081051 | IQESQALAKR | 1718 | HLA-C*07:06 |
| 2470 | AFP | ENSG00000081051 | IQESQALAK | 1719 | HLA-A*03:01 |
| 2471 | AFP | ENSG00000081051 | IQESQALAK | 1719 | HLA-A*03:02 |
| 2472 | AFP | ENSG00000081051 | IQESQALAK | 1719 | HLA-A*11:01 |
| 2473 | AFP | ENSG00000081051 | IQESQALAK | 1719 | HLA-B*27:05 |
| 2474 | AFP | ENSG00000081051 | IQKLVLDV | 1720 | HLA-B*13:02 |
| 2475 | AFP | ENSG00000081051 | ISKTRAAL | 1721 | HLA-B*08:01 |
| 2476 | AFP | ENSG00000081051 | ISKTRAAL | 1721 | HLA-C*16:01 |
| 2477 | AFP | ENSG00000081051 | ISLADLATIF | 1722 | HLA-B*57:01 |
| 2478 | AFP | ENSG00000081051 | ISLADLATI | 1723 | HLA-A*23:01 |
| 2479 | AFP | ENSG00000081051 | ISLADLATI | 1723 | HLA-B*58:01 |
| 2480 | AFP | ENSG00000081051 | ITVTKLSQKF | 1724 | HLA-B*57:01 |
| 2481 | AFP | ENSG00000081051 | ITVTKLSQKF | 1724 | HLA-B*58:01 |
| 2482 | AFP | ENSG00000081051 | ITVTKLSQK | 1725 | HLA-A*03:01 |
| 2483 | AFP | ENSG00000081051 | ITVTKLSQK | 1725 | HLA-A*03:02 |
| 2484 | AFP | ENSG00000081051 | ITVTKLSQK | 1725 | HLA-A*11:01 |
| 2485 | AFP | ENSG00000081051 | ITVTKLSQK | 1725 | HLA-A*68:01 |
| 2486 | AFP | ENSG00000081051 | ITVTKLSQK | 1725 | HLA-C*07:06 |
| 2487 | AFP | ENSG00000081051 | KAATVTKEL | 1726 | HLA-A*23:01 |
| 2488 | AFP | ENSG00000081051 | KAATVTKEL | 1726 | HLA-A*24:02 |
| 2489 | AFP | ENSG00000081051 | KAATVTKEL | 1726 | HLA-A*32:01 |
| 2490 | AFP | ENSG00000081051 | KAATVTKEL | 1726 | HLA-B*07:02 |
| 2491 | AFP | ENSG00000081051 | KAATVTKEL | 1726 | HLA-B*15:01 |
| 2492 | AFP | ENSG00000081051 | KAATVTKEL | 1726 | HLA-B*15:03 |
| 2493 | AFP | ENSG00000081051 | KAATVTKEL | 1726 | HLA-B*40:01 |
| 2494 | AFP | ENSG00000081051 | KAATVTKEL | 1726 | HLA-B*40:02 |
| 2495 | AFP | ENSG00000081051 | KAATVTKEL | 1726 | HLA-B*46:01 |
| 2496 | AFP | ENSG00000081051 | KAATVTKEL | 1726 | HLA-B*57:01 |
| 2497 | AFP | ENSG00000081051 | KAATVTKEL | 1726 | HLA-B*58:01 |
| 2498 | AFP | ENSG00000081051 | KAATVTKEL | 1726 | HLA-C*01:02 |
| 2499 | AFP | ENSG00000081051 | KAATVTKEL | 1726 | HLA-C*02:02 |
| 2500 | AFP | ENSG00000081051 | KAATVTKEL | 1726 | HLA-C*03:03 |
| 2501 | AFP | ENSG00000081051 | KAATVTKEL | 1726 | HLA-C*03:04 |
| 2502 | AFP | ENSG00000081051 | KAATVTKEL | 1726 | HLA-C*06:02 |
| 2503 | AFP | ENSG00000081051 | KAATVTKEL | 1726 | HLA-C*07:02 |
| 2504 | AFP | ENSG00000081051 | KAATVTKEL | 1726 | HLA-C*12:03 |
| 2505 | AFP | ENSG00000081051 | KAATVTKEL | 1726 | HLA-C*14:02 |
| 2506 | AFP | ENSG00000081051 | KAATVTKEL | 1726 | HLA-C*16:01 |
| 2507 | AFP | ENSG00000081051 | KAATVTKEL | 1726 | HLA-C*16:02 |
| 2508 | AFP | ENSG00000081051 | KAATVTKEL | 1726 | HLA-C*16:04 |
| 2509 | AFP | ENSG00000081051 | KAENAVECF | 1727 | HLA-B*58:01 |
| 2510 | AFP | ENSG00000081051 | KAENAVECF | 1727 | HLA-C*05:01 |
| 2511 | AFP | ENSG00000081051 | KAENAVECF | 1727 | HLA-C*07:04 |
| 2512 | AFP | ENSG00000081051 | KAPQLTSSEL | 1728 | HLA-B*07:02 |
| 2513 | AFP | ENSG00000081051 | KAPQLTSSEL | 1728 | HLA-B*58:01 |
| 2514 | AFP | ENSG00000081051 | KAPQLTSSEL | 1728 | HLA-C*01:02 |
| 2515 | AFP | ENSG00000081051 | KDALTAIEK | 1729 | HLA-A*03:02 |
| 2516 | AFP | ENSG00000081051 | KDLCQAQGV | 1730 | HLA-B*37:01 |
| 2517 | AFP | ENSG00000081051 | KELRESSLL | 1731 | HLA-B*37:01 |
| 2518 | AFP | ENSG00000081051 | KELRESSLL | 1731 | HLA-B*40:01 |
| 2519 | AFP | ENSG00000081051 | KELRESSLL | 1731 | HLA-B*40:02 |
| 2520 | AFP | ENSG00000081051 | KELRESSL | 1732 | HLA-B*37:01 |
| 2521 | AFP | ENSG00000081051 | KFIYEIARR | 1733 | HLA-A*31:01 |
| 2522 | AFP | ENSG00000081051 | KGEEELQKY | 1734 | HLA-A*01:01 |
| 2523 | AFP | ENSG00000081051 | KGEEELQKY | 1734 | HLA-A*30:02 |
| 2524 | AFP | ENSG00000081051 | KGYQELLEK | 1735 | HLA-A*03:01 |
| 2525 | AFP | ENSG00000081051 | KGYQELLEK | 1735 | HLA-A*03:02 |
| 2526 | AFP | ENSG00000081051 | KGYQELLEK | 1735 | HLA-A*11:01 |
| 2527 | AFP | ENSG00000081051 | KKAPQLTSSEL | 1736 | HLA-B*15:03 |
| 2528 | AFP | ENSG00000081051 | KLSQKFTKV | 1737 | HLA-A*02:03 |
| 2529 | AFP | ENSG00000081051 | KLVLDVAHV | 1738 | HLA-A*02:01 |
| 2530 | AFP | ENSG00000081051 | KLVLDVAHV | 1738 | HLA-A*02:03 |
| 2531 | AFP | ENSG00000081051 | KLVLDVAHV | 1738 | HLA-A*02:04 |
| 2532 | AFP | ENSG00000081051 | KLVLDVAHV | 1738 | HLA-A*02:07 |
| 2533 | AFP | ENSG00000081051 | KMAATAATC | 1739 | HLA-A*02:01 |
| 2534 | AFP | ENSG00000081051 | KMAATAATC | 1739 | HLA-B*55:01 |
| 2535 | AFP | ENSG00000081051 | KMVKDALTAI | 1740 | HLA-A*02:03 |
| 2536 | AFP | ENSG00000081051 | KPEGLSPNL | 1741 | HLA-B*07:02 |
| 2537 | AFP | ENSG00000081051 | KPEGLSPNL | 1741 | HLA-B*35:03 |
| 2538 | AFP | ENSG00000081051 | KPEGLSPNL | 1741 | HLA-C*07:02 |
| 2539 | AFP | ENSG00000081051 | KPQITEEQLEA | 1742 | HLA-B*56:01 |
| 2540 | AFP | ENSG00000081051 | KPQITEEQL | 1743 | HLA-B*07:02 |
| 2541 | AFP | ENSG00000081051 | KPQITEEQL | 1743 | HLA-B*35:03 |
| 2542 | AFP | ENSG00000081051 | KPQITEEQL | 1743 | HLA-C*07:02 |
| 2543 | AFP | ENSG00000081051 | KPTPASIPLF | 1744 | HLA-A*23:01 |
| 2544 | AFP | ENSG00000081051 | KPTPASIPLF | 1744 | HLA-A*24:02 |
| 2545 | AFP | ENSG00000081051 | KPTPASIPL | 1745 | HLA-B*07:02 |
| 2546 | AFP | ENSG00000081051 | KPTPASIPL | 1745 | HLA-B*35:03 |
| 2547 | AFP | ENSG00000081051 | KPTPASIPL | 1745 | HLA-C*07:02 |
| 2548 | AFP | ENSG00000081051 | KTRAALGV | 1746 | HLA-C*06:02 |
| 2549 | AFP | ENSG00000081051 | KVNFTEIQKL | 1747 | HLA-A*03:01 |
| 2550 | AFP | ENSG00000081051 | KVNFTEIQK | 1748 | HLA-A*03:01 |
| 2551 | AFP | ENSG00000081051 | KVNFTEIQK | 1748 | HLA-A*03:02 |
| 2552 | AFP | ENSG00000081051 | KVNFTEIQK | 1748 | HLA-A*11:01 |
| 2553 | AFP | ENSG00000081051 | KWVESIFLIF | 1749 | HLA-A*24:02 |
| 2554 | AFP | ENSG00000081051 | KWVESIFLI | 1750 | HLA-A*24:02 |
| 2555 | AFP | ENSG00000081051 | KYIQESQALAK | 1751 | HLA-A*03:01 |
| 2556 | AFP | ENSG00000081051 | KYIQESQALAK | 1751 | HLA-A*03:02 |
| 2557 | AFP | ENSG00000081051 | KYIQESQALAK | 1751 | HLA-A*11:01 |
| 2558 | AFP | ENSG00000081051 | KYIQESQALAK | 1751 | HLA-A*31:01 |
| 2559 | AFP | ENSG00000081051 | KYIQESQALAK | 1751 | HLA-A*33:03 |
| 2560 | AFP | ENSG00000081051 | KYIQESQAL | 1752 | HLA-A*23:01 |
| 2561 | AFP | ENSG00000081051 | KYIQESQAL | 1752 | HLA-A*24:02 |
| 2562 | AFP | ENSG00000081051 | KYIQESQAL | 1752 | HLA-A*30:01 |
| 2563 | AFP | ENSG00000081051 | KYIQESQAL | 1752 | HLA-A*31:01 |
| 2564 | AFP | ENSG00000081051 | KYIQESQAL | 1752 | HLA-A*32:01 |
| 2565 | AFP | ENSG00000081051 | KYIQESQAL | 1752 | HLA-B*15:01 |
| 2566 | AFP | ENSG00000081051 | KYIQESQAL | 1752 | HLA-B*15:03 |
| 2567 | AFP | ENSG00000081051 | KYIQESQAL | 1752 | HLA-B*27:05 |
| 2568 | AFP | ENSG00000081051 | KYIQESQAL | 1752 | HLA-B*35:03 |
| 2569 | AFP | ENSG00000081051 | KYIQESQAL | 1752 | HLA-B*38:01 |
| 2570 | AFP | ENSG00000081051 | KYIQESQAL | 1752 | HLA-B*40:01 |
| 2571 | AFP | ENSG00000081051 | KYIQESQAL | 1752 | HLA-B*40:02 |
| 2572 | AFP | ENSG00000081051 | KYIQESQAL | 1752 | HLA-B*46:01 |
| 2573 | AFP | ENSG00000081051 | KYIQESQAL | 1752 | HLA-B*58:01 |
| 2574 | AFP | ENSG00000081051 | KYIQESQAL | 1752 | HLA-C*01:02 |
| 2575 | AFP | ENSG00000081051 | KYIQESQAL | 1752 | HLA-C*03:03 |
| 2576 | AFP | ENSG00000081051 | KYIQESQAL | 1752 | HLA-C*03:04 |
| 2577 | AFP | ENSG00000081051 | KYIQESQAL | 1752 | HLA-C*04:01 |
| 2578 | AFP | ENSG00000081051 | KYIQESQAL | 1752 | HLA-C*06:02 |
| 2579 | AFP | ENSG00000081051 | KYIQESQAL | 1752 | HLA-C*07:02 |
| 2580 | AFP | ENSG00000081051 | KYIQESQAL | 1752 | HLA-C*14:02 |
| 2581 | AFP | ENSG00000081051 | KYIQESQAL | 1752 | HLA-C*16:01 |
| 2582 | AFP | ENSG00000081051 | KYIQESQAL | 1752 | HLA-C*16:02 |
| 2583 | AFP | ENSG00000081051 | KYIQESQAL | 1752 | HLA-C*16:04 |
| 2584 | AFP | ENSG00000081051 | LADLATIFF | 1753 | HLA-A*01:01 |
| 2585 | AFP | ENSG00000081051 | LADLATIFF | 1753 | HLA-A*02:07 |
| 2586 | AFP | ENSG00000081051 | LADLATIFF | 1753 | HLA-B*35:01 |
| 2587 | AFP | ENSG00000081051 | LADLATIFF | 1753 | HLA-B*38:01 |
| 2588 | AFP | ENSG00000081051 | LADLATIFF | 1753 | HLA-C*02:02 |
| 2589 | AFP | ENSG00000081051 | LADLATIFF | 1753 | HLA-C*04:01 |
| 2590 | AFP | ENSG00000081051 | LADLATIFF | 1753 | HLA-C*05:01 |
| 2591 | AFP | ENSG00000081051 | LADLATIF | 1754 | HLA-B*35:01 |
| 2592 | AFP | ENSG00000081051 | LADLATIF | 1754 | HLA-C*05:01 |
| 2593 | AFP | ENSG00000081051 | LAHKKPTPA | 1755 | HLA-B*54:01 |
| 2594 | AFP | ENSG00000081051 | LASFVHEY | 1756 | HLA-A*01:01 |
| 2595 | AFP | ENSG00000081051 | LASFVHEY | 1756 | HLA-A*29:02 |
| 2596 | AFP | ENSG00000081051 | LASFVHEY | 1756 | HLA-A*30:02 |
| 2597 | AFP | ENSG00000081051 | LASFVHEY | 1756 | HLA-A*32:01 |
| 2598 | AFP | ENSG00000081051 | LASFVHEY | 1756 | HLA-B*15:01 |
| 2599 | AFP | ENSG00000081051 | LASFVHEY | 1756 | HLA-B*15:03 |
| 2600 | AFP | ENSG00000081051 | LASFVHEY | 1756 | HLA-B*18:01 |
| 2601 | AFP | ENSG00000081051 | LASFVHEY | 1756 | HLA-B*35:01 |
| 2602 | AFP | ENSG00000081051 | LASFVHEY | 1756 | HLA-B*39:01 |
| 2603 | AFP | ENSG00000081051 | LASFVHEY | 1756 | HLA-B*46:01 |
| 2604 | AFP | ENSG00000081051 | LASFVHEY | 1756 | HLA-B*58:01 |
| 2605 | AFP | ENSG00000081051 | LASFVHEY | 1756 | HLA-C*01:02 |
| 2606 | AFP | ENSG00000081051 | LASFVHEY | 1756 | HLA-C*02:02 |
| 2607 | AFP | ENSG00000081051 | LASFVHEY | 1756 | HLA-C*03:03 |
| 2608 | AFP | ENSG00000081051 | LASFVHEY | 1756 | HLA-C*07:01 |
| 2609 | AFP | ENSG00000081051 | LASFVHEY | 1756 | HLA-C*07:04 |
| 2610 | AFP | ENSG00000081051 | LASFVHEY | 1756 | HLA-C*12:03 |
| 2611 | AFP | ENSG00000081051 | LASFVHEY | 1756 | HLA-C*16:01 |
| 2612 | AFP | ENSG00000081051 | LASFVHEY | 1756 | HLA-C*16:02 |
| 2613 | AFP | ENSG00000081051 | LASFVHEY | 1756 | HLA-C*16:04 |
| 2614 | AFP | ENSG00000081051 | LATIFFAQF | 1757 | HLA-B*57:01 |
| 2615 | AFP | ENSG00000081051 | LATIGGAQF | 1757 | HLA-C*02:02 |
| 2616 | AFP | ENSG00000081051 | LAVSVILRVA | 1758 | HLA-B*54:01 |
| 2617 | AFP | ENSG00000081051 | LAVSVILRV | 1759 | HLA-A*02:04 |
| 2618 | AFP | ENSG00000081051 | LAVSVILRV | 1759 | HLA-A*02:07 |
| 2619 | AFP | ENSG00000081051 | LAVSVILRV | 1759 | HLA-A*68:02 |
| 2620 | AFP | ENSG00000081051 | LAVSVILRV | 1759 | HLA-B*13:02 |
| 2621 | AFP | ENSG00000081051 | LAVSVILRV | 1759 | HLA-B*51:01 |
| 2622 | AFP | ENSG00000081051 | LAVSVILRV | 1759 | HLA-B*54:01 |
| 2623 | AFP | ENSG00000081051 | LAVSVILRV | 1759 | HLA-C*02:02 |
| 2624 | AFP | ENSG00000081051 | LAVSVILRV | 1759 | HLA-C*12:03 |
| 2625 | AFP | ENSG00000081051 | LCQAQGVAL | 1760 | HLA-C*01:02 |
| 2626 | AFP | ENSG00000081051 | LCQAQGVAL | 1760 | HLA-C*03:03 |
| 2627 | AFP | ENSG00000081051 | LCQAQGVAL | 1760 | HLA-C*03:04 |
| 2628 | AFP | ENSG00000081051 | LCQAQGVAL | 1760 | HLA-C*14:02 |
| 2629 | AFP | ENSG00000081051 | LEAVIADF | 1761 | HLA-B*18:01 |
| 2630 | AFP | ENSG00000081051 | LEAVIADF | 1761 | HLA-B*37:01 |
| 2631 | AFP | ENSG00000081051 | LENQLPAFL | 1762 | HLA-B*18:01 |
| 2632 | AFP | ENSG00000081051 | LENQLPAFL | 1762 | HLA-B*40:01 |
| 2633 | AFP | ENSG00000081051 | LENQLPAFL | 1762 | HLA-B*44:02 |
| 2634 | AFP | ENSG00000081051 | LENQLPAFL | 1762 | HLA-B*44:03 |
| 2635 | AFP | ENSG00000081051 | LENQLPAFL | 1762 | HLA-B*49:01 |
| 2636 | AFP | ENSG00000081051 | LENQLPAF | 1763 | HLA-B*18:01 |
| 2637 | AFP | ENSG00000081051 | LENQLPAF | 1763 | HLA-B*37:01 |
| 2638 | AFP | ENSG00000081051 | LFQKLGEYY | 1764 | HLA-A*29:02 |
| 2639 | AFP | ENSG00000081051 | LFQKLGEYY | 1764 | HLA-A*30:02 |
| 2640 | AFP | ENSG00000081051 | LFQKLGEY | 1765 | HLA-A*30:02 |
| 2641 | AFP | ENSG00000081051 | LFQKLGEY | 1765 | HLA-C*14:02 |
| 2642 | AFP | ENSG00000081051 | LGDRDFNQF | 1766 | HLA-A*01:01 |
| 2643 | AFP | ENSG00000081051 | LGDRDFNQF | 1766 | HLA-B*38:01 |
| 2644 | AFP | ENSG00000081051 | LGDRDFNQF | 1766 | HLA-C*05:01 |
| 2645 | AFP | ENSG00000081051 | LLNQHACAV | 1767 | HLA-A*02:01 |
| 2646 | AFP | ENSG00000081051 | LLNQHACAV | 1767 | HLA-A*02:03 |
| 2647 | AFP | ENSG00000081051 | LLNQHACAV | 1767 | HLA-B*55:01 |
| 2648 | AFP | ENSG00000081051 | LNFTESRTL | 1768 | HLA-C*03:03 |
| 2649 | AFP | ENSG00000081051 | LNFTESRTL | 1768 | HLA-C*03:04 |
| 2650 | AFP | ENSG00000081051 | LNFTESRTL | 1768 | HLA-C*I6:01 |
| 2651 | AFP | ENSG00000081051 | LPAFLEEL | 1769 | HLA-B*35:01 |
| 2652 | AFP | ENSG00000081051 | LPAFLEEL | 1769 | HLA-B*35:03 |
| 2653 | AFP | ENSG00000081051 | LPAFLEEL | 1769 | HLA-B*51:01 |
| 2654 | AFP | ENSG00000081051 | LPAFLEEL | 1769 | HLA-B*56:01 |
| 2655 | AFP | ENSG00000081051 | LQDGEKIMSY | 1770 | HLA-A*01:01 |
| 2656 | AFP | ENSG00000081051 | LQDGEKIMSY | 1770 | HLA-A*30:02 |
| 2657 | AFP | ENSG00000081051 | LQDGEKIMSY | 1770 | HLA-B*15:01 |
| 2658 | AFP | ENSG00000081051 | LQDGEKIMSY | 1770 | HLA-C*07:04 |
| 2659 | AFP | ENSG00000081051 | LQDGEKIM | 1771 | HLA-C*05:01 |
| 2660 | AFP | ENSG00000081051 | LQNAFLVAY | 1772 | HLA-A*03:01 |
| 2661 | AFP | ENSG00000081051 | LQNAFLVAY | 1772 | HLA-A*26:01 |
| 2662 | AFP | ENSG00000081051 | LQNAFLVAY | 1772 | HLA-A*29:02 |
| 2663 | AFP | ENSG00000081051 | LQNAFLVAY | 1772 | HLA-A*30:02 |
| 2664 | AFP | ENSG00000081051 | LQNAFLVAY | 1772 | HLA-A*31:01 |
| 2665 | AFP | ENSG00000081051 | LQNAFLVAY | 1772 | HLA-A*32:01 |
| 2666 | AFP | ENSG00000081051 | LQNAFLVAY | 1772 | HLA-B*15:01 |
| 2667 | AFP | ENSG00000081051 | LQNAFLVAY | 1772 | HLA-B*15:03 |
| 2668 | AFP | ENSG00000081051 | LQNAFLVAY | 1772 | HLA-B*18:01 |
| 2669 | AFP | ENSG00000081051 | LQNAFLVAY | 1772 | HLA-B*27:02 |
| 2670 | AFP | ENSG00000081051 | LQNAFLVAY | 1772 | HLA-B*27:05 |
| 2671 | AFP | ENSG00000081051 | LQNAFLVAY | 1772 | HLA-B*35:01 |
| 2672 | AFP | ENSG00000081051 | LQNAFLVAY | 1772 | HLA-B*39:01 |
| 2673 | AFP | ENSG00000081051 | LQNAFLVAY | 1772 | HLA-B*44:02 |
| 2674 | AFP | ENSG00000081051 | LQNAFLVAY | 1772 | HLA-B*44:03 |
| 2675 | AFP | ENSG00000081051 | LQNAFLVAY | 1772 | HLA-B*46:01 |
| 2676 | AFP | ENSG00000081051 | LQNAFLVAY | 1772 | HLA-C*01:02 |
| 2677 | AFP | ENSG00000081051 | LQNAFLVAY | 1772 | HLA-C*02:02 |
| 2678 | AFP | ENSG00000081051 | LQNAFLVAY | 1772 | HLA-C*03:03 |
| 2679 | AFP | ENSG00000081051 | LQNAFLVAY | 1772 | HLA-C*07:01 |
| 2680 | AFP | ENSG00000081051 | LQNAFLVAY | 1772 | HLA-C*07:04 |
| 2681 | AFP | ENSG00000081051 | LQNAFLVAY | 1772 | HLA-C*12:03 |
| 2682 | AFP | ENSG00000081051 | LQNAFLVAY | 1772 | HLA-C*14:02 |
| 2683 | AFP | ENSG00000081051 | LQNAFLVAY | 1772 | HLA-C*16:01 |
| 2684 | AFP | ENSG00000081051 | LQNAFLVAY | 1772 | HLA-C*16:02 |
| 2685 | AFP | ENSG00000081051 | LQNAFLVAY | 1772 | HLA-C*16:04 |
| 2686 | AFP | ENSG00000081051 | LQTIVEKQEF | 1773 | HLA-B*15:01 |
| 2687 | AFP | ENSG00000081051 | LSQKFTKVNF | 1774 | HLA-B*57:01 |
| 2688 | AFP | ENSG00000081051 | LSQKFTKVNF | 1774 | HLA-B*58:01 |
| 2689 | AFP | ENSG00000081051 | LTSSELMAITR | 1775 | HLA-A*33:03 |
| 2690 | AFP | ENSG00000081051 | LTSSELMAITR | 1775 | HLA-A*68:01 |
| 2691 | AFP | ENSG00000081051 | LTSSELMAITR | 1775 | HLA-B*27:02 |
| 2692 | AFP | ENSG00000081051 | LTSSELMAITR | 1775 | HLA-C*07:06 |
| 2693 | AFP | ENSG00000081051 | LVKQKPQI | 1776 | HLA-B*08:01 |
| 2694 | AFP | ENSG00000081051 | LYAPTILLW | 1777 | HLA-A*23:01 |
| 2695 | AFP | ENSG00000081051 | LYAPTILLW | 1777 | HLA-A*24:02 |
| 2696 | AFP | ENSG00000081051 | LYAPTILLW | 1777 | HLA-A*29:02 |
| 2697 | AFP | ENSG00000081051 | LYAPTILLW | 1777 | HLA-B*57:01 |
| 2698 | AFP | ENSG00000081051 | LYAPTILL | 1778 | HLA-A*23:01 |
| 2699 | AFP | ENSG00000081051 | LYAPTILL | 1778 | HLA-A*24:02 |
| 2700 | AFP | ENSG00000081051 | MAATAATCC | 1779 | HLA-B*51:01 |
| 2701 | AFP | ENSG00000081051 | MAATAATCC | 1779 | HLA-B*54:01 |
| 2702 | AFP | ENSG00000081051 | MAATAATCC | 1779 | HLA-C*03:03 |
| 2703 | AFP | ENSG00000081051 | MAATAATCC | 1779 | HLA-C*03:04 |
| 2704 | AFP | ENSG00000081051 | MAATAATC | 1780 | HLA-B*51:01 |
| 2705 | AFP | ENSG00000081051 | MAATAATC | 1780 | HLA-C*03:04 |
| 2706 | AFP | ENSG00000081051 | MAITRKMAA | 1781 | HLA-B*08:01 |
| 2707 | AFP | ENSG00000081051 | MAITRKMAA | 1781 | HLA-B*54:01 |
| 2708 | AFP | ENSG00000081051 | MAITRKMAA | 1781 | HLA-C*16:01 |
| 2709 | AFP | ENSG00000081051 | MVKDALTAIEK | 1782 | HLA-A*03:01 |
| 2710 | AFP | ENSG00000081051 | MVKDALTAIEK | 1782 | HLA-A*03:02 |
| 2711 | AFP | ENSG00000081051 | MVKDALTAIEK | 1782 | HLA-A*11:01 |
| 2712 | AFP | ENSG00000081051 | MVKDALTAIEK | 1782 | HLA-A*31:01 |
| 2713 | AFP | ENSG00000081051 | MVKDALTAIEK | 1782 | HLA-A*33:01 |
| 2714 | AFP | ENSG00000081051 | MVKDALTAIEK | 1782 | HLA-A*33:03 |
| 2715 | AFP | ENSG00000081051 | MVKDALTAIEK | 1782 | HLA-A*68:01 |
| 2716 | AFP | ENSG00000081051 | MVKDALTAI | 1783 | HLA-A*02:03 |
| 2717 | AFP | ENSG00000081051 | MVKDALTAI | 1783 | HLA-A*23:01 |
| 2718 | AFP | ENSG00000081051 | MVKDALTAI | 1783 | HLA-A*25:01 |
| 2719 | AFP | ENSG00000081051 | MVKDALTAI | 1783 | HLA-A*26:01 |
| 2720 | AFP | ENSG00000081051 | MVKDALTAI | 1783 | HLA-A*32:01 |
| 2721 | AFP | ENSG00000081051 | MVKDALTAI | 1783 | HLA-A*33:03 |
| 2722 | AFP | ENSG00000081051 | MVKDALTAI | 1783 | HLA-A*68:02 |
| 2723 | AFP | ENSG00000081051 | MVKDALTAI | 1783 | HLA-B*08:01 |
| 2724 | AFP | ENSG00000081051 | MVKDALTAI | 1783 | HLA-B*40:02 |
| 2725 | AFP | ENSG00000081051 | MVKDALTAI | 1783 | HLA-B*46:01 |
| 2726 | AFP | ENSG00000081051 | MVKDALTAI | 1783 | HLA-B*51:01 |
| 2727 | AFP | ENSG00000081051 | MVKDALTAI | 1783 | HLA-B*54:01 |
| 2728 | AFP | ENSG00000081051 | MVKDALTAI | 1783 | HLA-C*01:02 |
| 2729 | AFP | ENSG00000081051 | MVKDALTAI | 1783 | HLA-C*02:02 |
| 2730 | AFP | ENSG00000081051 | MVKDALTAI | 1783 | HLA-C*03:03 |
| 2731 | AFP | ENSG00000081051 | MVKDALTAI | 1783 | HLA-C*03:04 |
| 2732 | AFP | ENSG00000081051 | MVKDALTAI | 1783 | HLA-C*07:04 |
| 2733 | AFP | ENSG00000081051 | MVKDALTAI | 1783 | HLA-C*14:02 |
| 2734 | AFP | ENSG00000081051 | NAFLVAYTK | 1784 | HLA-A*11:01 |
| 2735 | AFP | ENSG00000081051 | NAFLVAYTK | 1784 | HLA-A*68:01 |
| 2736 | AFP | ENSG00000081051 | NAFLVAYTK | 1784 | HLA-B*35:01 |
| 2737 | AFP | ENSG00000081051 | NAFLVAYTK | 1784 | HLA-C*07:06 |
| 2738 | AFP | ENSG00000081051 | NAVECFQTK | 1785 | HLA-A*33:01 |
| 2739 | AFP | ENSG00000081051 | NAVECFQTK | 1785 | HLA-A*68:01 |
| 2740 | AFP | ENSG00000081051 | NAVECFQTK | 1785 | HLA-C*07:06 |
| 2741 | AFP | ENSG00000081051 | NEYGIASIL | 1786 | HLA-A*30:01 |
| 2742 | AFP | ENSG00000081051 | NEYGIASIL | 1786 | HLA-B*18:01 |
| 2743 | AFP | ENSG00000081051 | NEYGIASIL | 1786 | HLA-B*37:01 |
| 2744 | AFP | ENSG00000081051 | NEYGIASIL | 1786 | HLA-B*39:01 |
| 2745 | AFP | ENSG00000081051 | NEYGIASIL | 1786 | HLA-B*40:01 |
| 2746 | AFP | ENSG00000081051 | NEYGIASIL | 1786 | HLA-B*40:02 |
| 2747 | AFP | ENSG00000081051 | NEYGIASIL | 1786 | HLA-B*44:02 |
| 2748 | AFP | ENSG00000081051 | NEYGIASIL | 1786 | HLA-B*44:03 |
| 2749 | AFP | ENSG00000081051 | NEYGIASIL | 1786 | HLA-B*49:01 |
| 2750 | AFP | ENSG00000081051 | NEYGIASIL | 1786 | HLA-C*02:02 |
| 2751 | AFP | ENSG00000081051 | NEYGIASIL | 1786 | HLA-C*12:03 |
| 2752 | AFP | ENSG00000081051 | NEYGIASIL | 1786 | HLA-C*14:02 |
| 2753 | AFP | ENSG00000081051 | NEYGIASIL | 1786 | HLA-C*16:04 |
| 2754 | AFP | ENSG00000081051 | NEYGIASI | 1787 | HLA-A*30:01 |
| 2755 | AFP | ENSG00000081051 | NEYGIASI | 1787 | HLA-B*18:01 |
| 2756 | AFP | ENSG00000081051 | NEYGIASI | 1787 | HLA-B*37:01 |
| 2757 | AFP | ENSG00000081051 | NEYGIASI | 1787 | HLA-B*49:01 |
| 2758 | AFP | ENSG00000081051 | NFTEIQKL | 1788 | HLA-C*14:02 |
| 2759 | AFP | ENSG00000081051 | NFTESRTL | 1789 | HLA-C*01:02 |
| 2760 | AFP | ENSG00000081051 | NFTESRTL | 1789 | HLA-C*14:02 |
| 2761 | AFP | ENSG00000081051 | NFTESRTL | 1789 | HLA-C*16:01 |
| 2762 | AFP | ENSG00000081051 | NIFLASFVHEY | 1790 | HLA-A*29:02 |
| 2763 | AFP | ENSG00000081051 | NQFSSGEKNIF | 1791 | HLA-B*38:01 |
| 2764 | AFP | ENSG00000081051 | NQFSSGEKNI | 1792 | HLA-B*13:02 |
| 2765 | AFP | ENSG00000081051 | PFLYAPTI | 1793 | HLA-A*23:01 |
| 2766 | AFP | ENSG00000081051 | PTPASIPLF | 1794 | HLA-A*24:02 |
| 2767 | AFP | ENSG00000081051 | PTPASIPLF | 1794 | HLA-A*26:01 |
| 2768 | AFP | ENSG00000081051 | QAITVTKLSQK | 1795 | HLA-A*03:02 |
| 2769 | AFP | ENSG00000081051 | QAITVTKLSQK | 1795 | HLA-A*11:01 |
| 2770 | AFP | ENSG00000081051 | QAITVTKLSQK | 1795 | HLA-A*31:01 |
| 2771 | AFP | ENSG00000081051 | QAITVTKLSQK | 1795 | HLA-A*68:01 |
| 2772 | AFP | ENSG00000081051 | QAITVTKLSQK | 1795 | HLA-C*07:06 |
| 2773 | AFP | ENSG00000081051 | QAITVTKL | 1796 | HLA-A*23:01 |
| 2774 | AFP | ENSG00000081051 | QAITVTKL | 1796 | HLA-A*24:02 |
| 2775 | AFP | ENSG00000081051 | QAITVTKL | 1796 | HLA-A*30:01 |
| 2776 | AFP | ENSG00000081051 | QAITVTKL | 1796 | HLA-A*32:01 |
| 2777 | AFP | ENSG00000081051 | QAITVTKL | 1796 | HLA-B*08:01 |
| 2778 | AFP | ENSG00000081051 | QAITVTKL | 1796 | HLA-B*15:01 |
| 2779 | AFP | ENSG00000081051 | QAITVTKL | 1796 | HLA-B*15:03 |
| 2780 | AFP | ENSG00000081051 | QAITVTKL | 1796 | HLA-B*18:01 |
| 2781 | AFP | ENSG00000081051 | QAITVTKL | 1796 | HLA-B*35:03 |
| 2782 | AFP | ENSG00000081051 | QAITVTKL | 1796 | HLA-B*40:01 |
| 2783 | AFP | ENSG00000081051 | QAITVTKL | 1796 | HLA-B*40:02 |
| 2784 | AFP | ENSG00000081051 | QAITVTKL | 1796 | HLA-B*46:01 |
| 2785 | AFP | ENSG00000081051 | QAITVTKL | 1796 | HLA-B*51:01 |
| 2786 | AFP | ENSG00000081051 | QAITVTKL | 1796 | HLA-B*58:01 |
| 2787 | AFP | ENSG00000081051 | QAITVTKL | 1796 | HLA-C*01:02 |
| 2788 | AFP | ENSG00000081051 | QAITVTKL | 1796 | HLA-C*02:02 |
| 2789 | AFP | ENSG00000081051 | QAITVTKL | 1796 | HLA-C*03:03 |
| 2790 | AFP | ENSG00000081051 | QAITVTKL | 1796 | HLA-C*03:04 |
| 2791 | AFP | ENSG00000081051 | QAITVTKL | 1796 | HLA-C*05:01 |
| 2792 | AFP | ENSG00000081051 | QAITVTKL | 1796 | HLA-C*06:02 |
| 2793 | AFP | ENSG00000081051 | QAITVTKL | 1796 | HLA-C*07:01 |
| 2794 | AFP | ENSG00000081051 | QAITVTKL | 1796 | HLA-C*12:03 |
| 2795 | AFP | ENSG00000081051 | QAITVTKL | 1796 | HLA-C*16:01 |
| 2796 | AFP | ENSG00000081051 | QAITVTKL | 1796 | HLA-C*16:02 |
| 2797 | AFP | ENSG00000081051 | QAQGVALQTMK | 1797 | HLA-A*03:02 |
| 2798 | AFP | ENSG00000081051 | QAQGVALQTMK | 1797 | HLA-A*11:01 |
| 2799 | AFP | ENSG00000081051 | QAQGVALQTMK | 1797 | HLA-A*68:01 |
| 2800 | AFP | ENSG00000081051 | QAQGVALQTMK | 1797 | HLA-B*27:02 |
| 2801 | AFP | ENSG00000081051 | QAQGVALQTM | 1798 | HLA-B*35:01 |
| 2802 | AFP | ENSG00000081051 | QAQGVALQTM | 1798 | HLA-B*35:03 |
| 2803 | AFP | ENSG00000081051 | QAQGVALQTM | 1798 | HLA-B*58:01 |
| 2804 | AFP | ENSG00000081051 | QAQGVALQTM | 1798 | HLA-C*03:04 |
| 2805 | AFP | ENSG00000081051 | QAQGVALQTM | 1798 | HLA-C*04:01 |
| 2806 | AFP | ENSG00000081051 | QAQGVALQTM | 1798 | HLA-C*07:06 |
| 2807 | AFP | ENSG00000081051 | QAQGVALQTM | 1798 | HLA-C*16:02 |
| 2808 | AFP | ENSG00000081051 | QDGEKIMSY | 1799 | HLA-B*18:01 |
| 2809 | AFP | ENSG00000081051 | QDGEKIMSY | 1799 | HLA-B*37:01 |
| 2810 | AFP | ENSG00000081051 | QDGEKIMSY | 1799 | HLA-B*44:02 |
| 2811 | AFP | ENSG00000081051 | QDGEKIMSY | 1799 | HLA-B*44:03 |
| 2812 | AFP | ENSG00000081051 | QDGEKIMSY | 1799 | HLA-C*12:03 |
| 2813 | AFP | ENSG00000081051 | QDTLSNKI | 1800 | HLA-B*13:02 |
| 2814 | AFP | ENSG00000081051 | QDTLSNKI | 1800 | HLA-B*37:01 |
| 2815 | AFP | ENSG00000081051 | QDTLSNKI | 1800 | HLA-B*49:01 |
| 2816 | AFP | ENSG00000081051 | QDTLSNKI | 1800 | HLA-C*16:02 |
| 2817 | AFP | ENSG00000081051 | QEATYKEV | 1801 | HLA-B*49:01 |
| 2818 | AFP | ENSG00000081051 | QEFLINLV | 1802 | HLA-A*30:01 |
| 2819 | AFP | ENSG00000081051 | QEFLINLV | 1802 | HLA-B*18:01 |
| 2820 | AFP | ENSG00000081051 | QEFLINLV | 1802 | HLA-B*37:01 |
| 2821 | AFP | ENSG00000081051 | QEFLINLV | 1802 | HLA-B*49:01 |
| 2822 | AFP | ENSG00000081051 | QESQALAKR | 1803 | HLA-B*44:02 |
| 2823 | AFP | ENSG00000081051 | QESQALAKR | 1803 | HLA-B*44:03 |
| 2824 | AFP | ENSG00000081051 | QESQALAKR | 1803 | HLA-C*16:04 |
| 2825 | AFP | ENSG00000081051 | QFVQEATY | 1804 | HLA-C*14:02 |
| 2826 | AFP | ENSG00000081051 | QGVALQTMK | 1805 | HLA-B*27:02 |
| 2827 | AFP | ENSG00000081051 | QGVALQTM | 1806 | HLA-B*51:01 |
| 2828 | AFP | ENSG00000081051 | QHACAVMKNF | 1807 | HLA-B*38:01 |
| 2829 | AFP | ENSG00000081051 | QKFTKVNF | 1808 | HLA-B*15:03 |
| 2830 | AFP | ENSG00000081051 | QKYIQESQAL | 1809 | HLA-B*15:03 |
| 2831 | AFP | ENSG00000081051 | QLAVSVILRV | 1810 | HLA-A*02:03 |
| 2832 | AFP | ENSG00000081051 | QLAVSVILR | 1811 | HLA-A*68:01 |
| 2833 | AFP | ENSG00000081051 | QLAVSVILR | 1811 | HLA-C*07:06 |
| 2834 | AFP | ENSG00000081051 | QLPAFLEEL | 1812 | HLA-A*02:01 |
| 2835 | AFP | ENSG00000081051 | QLPAFLEEL | 1812 | HLA-A*02:03 |
| 2836 | AFP | ENSG00000081051 | QLPAFLEEL | 1812 | HLA-A*02:04 |
| 2837 | AFP | ENSG00000081051 | QLPAFLEEL | 1812 | HLA-A*02:07 |
| 2838 | AFP | ENSG00000081051 | QLPAFLEEL | 1812 | HLA-A*24:02 |
| 2839 | AFP | ENSG00000081051 | QLPAFLEEL | 1812 | HLA-C*01:02 |
| 2840 | AFP | ENSG00000081051 | QLSEDKLLAC | 1813 | HLA-A*02:01 |
| 2841 | AFP | ENSG00000081051 | QLSEDKLLA | 1814 | HLA-A*02:01 |
| 2842 | AFP | ENSG00000081051 | QLSEDKLLA | 1814 | HLA-B*13:02 |
| 2843 | AFP | ENSG00000081051 | QNAFLVAYTK | 1815 | HLA-B*27:02 |
| 2844 | AFP | ENSG00000081051 | QQDTLSNKI | 1816 | HLA-B*13:02 |
| 2845 | AFP | ENSG00000081051 | QQDTLSNKI | 1816 | HLA-B*38:01 |
| 2846 | AFP | ENSG00000081051 | QQDTLSNKI | 1816 | HLA-B*39:01 |
| 2847 | AFP | ENSG00000081051 | QQDTLSNKI | 1816 | HLA-C*05:01 |
| 2848 | AFP | ENSG00000081051 | QQDTLSNKI | 1816 | HLA-C*06:02 |
| 2849 | AFP | ENSG00000081051 | QTKAATVTK | 1817 | HLA-A*03:01 |
| 2850 | AFP | ENSG00000081051 | QTKAATVTK | 1817 | HLA-A*03:02 |
| 2851 | AFP | ENSG00000081051 | QTKAATVTK | 1817 | HLA-A*11:01 |
| 2852 | AFP | ENSG00000081051 | QTKAATVTK | 1817 | HLA-A*31:01 |
| 2853 | AFP | ENSG00000081051 | QTKAATVTK | 1817 | HLA-A*33:01 |
| 2854 | AFP | ENSG00000081051 | QTKAATVTK | 1817 | HLA-A*33:03 |
| 2855 | AFP | ENSG00000081051 | QTKAATVTK | 1817 | HLA-A*68:01 |
| 2856 | AFP | ENSG00000081051 | QTKAATVTK | 1817 | HLA-C*07:06 |
| 2857 | AFP | ENSG00000081051 | QTMKQEFLINL | 1818 | HLA-A*31:01 |
| 2858 | AFP | ENSG00000081051 | QVPEPVTSC | 1819 | HLA-A*02:07 |
| 2859 | AFP | ENSG00000081051 | QVPEPVTSC | 1819 | HLA-A*25:01 |
| 2860 | AFP | ENSG00000081051 | QVPEPVTSC | 1819 | HLA-C*01:02 |
| 2861 | AFP | ENSG00000081051 | RETFMNKF | 1820 | HLA-B*37:01 |
| 2862 | AFP | ENSG00000081051 | RFLGDRDFNQF | 1821 | HLA-A*24:02 |
| 2863 | AFP | ENSG00000081051 | RTFQAITVTKL | 1822 | HLA-A*03:01 |
| 2864 | AFP | ENSG00000081051 | RTFQAITVTKL | 1822 | HLA-B*57:01 |
| 2865 | AFP | ENSG00000081051 | RTFQAITVTK | 1823 | HLA-A*03:01 |
| 2866 | AFP | ENSG00000081051 | RTFQAITVTK | 1823 | HLA-A*03:02 |
| 2867 | AFP | ENSG00000081051 | RTFQAITVTK | 1823 | HLA-A*11:01 |
| 2868 | AFP | ENSG00000081051 | RTFQAITVTK | 1823 | HLA-A*31:01 |
| 2869 | AFP | ENSG00000081051 | RTFQAITVTK | 1823 | HLA-B*27:02 |
| 2870 | AFP | ENSG00000081051 | RTFQAITVTK | 1823 | HLA-B*57:01 |
| 2871 | AFP | ENSG00000081051 | SEEGRHNCF | 1824 | HLA-B*37:01 |
| 2872 | AFP | ENSG00000081051 | SELMAITRKM | 1825 | HLA-B*44:02 |
| 2873 | AFP | ENSG00000081051 | SELMAITRKM | 1825 | HLA-B*44:03 |
| 2874 | AFP | ENSG00000081051 | SELMAITR | 1826 | HLA-B*18:01 |
| 2875 | AFP | ENSG00000081051 | SIFLIFLL | 1827 | HLA-A*02:04 |
| 2876 | AFP | ENSG00000081051 | SKMVKDAL | 1828 | HLA-B*08:01 |
| 2877 | AFP | ENSG00000081051 | SLADLATIFFA | 1829 | HLA-A*02:01 |
| 2878 | AFP | ENSG00000081051 | SLADLATIFFA | 1829 | HLA-A*02:04 |
| 2879 | AFP | ENSG00000081051 | SLADLATIFF | 1830 | HLA-A*02:01 |
| 2880 | AFP | ENSG00000081051 | SLADLATIFF | 1830 | HLA-A*02:04 |
| 2881 | AFP | ENSG00000081051 | SLADLATIF | 1831 | HLA-A*23:01 |
| 2882 | AFP | ENSG00000081051 | SLADLATIF | 1831 | HLA-A*24:02 |
| 2883 | AFP | ENSG00000081051 | SLADLATIF | 1831 | HLA-A*25:01 |
| 2884 | AFP | ENSG00000081051 | SLADLATIF | 1831 | HLA-A*26:01 |
| 2885 | AFP | ENSG00000081051 | SLADLATIF | 1831 | HLA-A*29:02 |
| 2886 | AFP | ENSG00000081051 | SLADLATIF | 1831 | HLA-A*32:01 |
| 2887 | AFP | ENSG00000081051 | SLADLATIF | 1831 | HLA-B*15:01 |
| 2888 | AFP | ENSG00000081051 | SLADLATIF | 1831 | HLA-B*15:03 |
| 2889 | AFP | ENSG00000081051 | SLADLATIF | 1831 | HLA-B*35:01 |
| 2890 | AFP | ENSG00000081051 | SLADLATIF | 1831 | HLA-B*44:02 |
| 2891 | AFP | ENSG00000081051 | SLADLATIF | 1831 | HLA-B*44:03 |
| 2892 | AFP | ENSG00000081051 | SLADLATIF | 1831 | HLA-B*46:01 |
| 2893 | AFP | ENSG00000081051 | SLADLATIF | 1831 | HLA-C*02:02 |
| 2894 | AFP | ENSG00000081051 | SLADLATIF | 1831 | HLA-C*07:04 |
| 2895 | AFP | ENSG00000081051 | SLADLATIF | 1831 | HLA-C*12:03 |
| 2896 | AFP | ENSG00000081051 | SLADLATI | 1832 | HLA-A*02:01 |
| 2897 | AFP | ENSG00000081051 | SLLNQHACAV | 1833 | HLA-A*02:01 |
| 2898 | AFP | ENSG00000081051 | SLLNQHACAV | 1833 | HLA-A*02:03 |
| 2899 | AFP | ENSG00000081051 | SLVVDETYV | 1834 | HLA-A*02:01 |
| 2900 | AFP | ENSG00000081051 | SLVVDETY | 1835 | HLA-B*15:01 |
| 2901 | AFP | ENSG00000081051 | SLVVDETY | 1835 | HLA-B*15:03 |
| 2902 | AFP | ENSG00000081051 | SPNLNRFL | 1836 | HLA-B*07:02 |
| 2903 | AFP | ENSG00000081051 | SQKFTKVNF | 1837 | HLA-A*32:01 |
| 2904 | AFP | ENSG00000081051 | SQKFTKVNF | 1837 | HLA-B*15:01 |
| 2905 | AFP | ENSG00000081051 | SQKFTKVNF | 1837 | HLA-B*15:03 |
| 2906 | AFP | ENSG00000081051 | SQKFTKVNF | 1837 | HLA-C*07:04 |
| 2907 | AFP | ENSG00000081051 | SQQDTLSNKI | 1838 | HLA-B*13:02 |
| 2908 | AFP | ENSG00000081051 | SQQDTLSNKI | 1838 | HLA-B*38:01 |
| 2909 | AFP | ENSG00000081051 | SQQDTLSNK | 1839 | HLA-A*03:02 |
| 2910 | AFP | ENSG00000081051 | SQQDTLSNK | 1839 | HLA-A*11:01 |
| 2911 | AFP | ENSG00000081051 | SSELMAITRK | 1840 | HLA-A*11:01 |
| 2912 | AFP | ENSG00000081051 | SSELMAITR | 1841 | HLA-A*11:01 |
| 2913 | AFP | ENSG00000081051 | SSELMAITR | 1841 | HLA-A*68:01 |
| 2914 | AFP | ENSG00000081051 | SSELMAITR | 1841 | HLA-C*07:06 |
| 2915 | AFP | ENSG00000081051 | SSGEKNIF | 1842 | HLA-C*16:01 |
| 2916 | AFP | ENSG00000081051 | SSLVVDETY | 1843 | HLA-A*01:01 |
| 2917 | AFP | ENSG00000081051 | SSLVVDETY | 1843 | HLA-A*30:02 |
| 2918 | AFP | ENSG00000081051 | SSLVVDETY | 1843 | HLA-B*15:01 |
| 2919 | AFP | ENSG00000081051 | SSLVVDETY | 1843 | HLA-B*15:03 |
| 2920 | AFP | ENSG00000081051 | SSLVVDETY | 1843 | HLA-B*35:01 |
| 2921 | AFP | ENSG00000081051 | SSLVVDETY | 1843 | HLA-B*46:01 |
| 2922 | AFP | ENSG00000081051 | SSLVVDETY | 1843 | HLA-B*57:01 |
| 2923 | AFP | ENSG00000081051 | SSLVVDETY | 1843 | HLA-B*58:01 |
| 2924 | AFP | ENSG00000081051 | SSLVVDETY | 1843 | HLA-C*01:02 |
| 2925 | AFP | ENSG00000081051 | SSLVVDETY | 1843 | HLA-C*16:04 |
| 2926 | AFP | ENSG00000081051 | SYANRRPCF | 1844 | HLA-A*24:02 |
| 2927 | AFP | ENSG00000081051 | SYICSQQDTL | 1845 | HLA-A*23:01 |
| 2928 | AFP | ENSG00000081051 | SYICSQQDTL | 1845 | HLA-A*24:02 |
| 2929 | AFP | ENSG00000081051 | SYICSQQDTL | 1845 | HLA-C*14:02 |
| 2930 | AFP | ENSG00000081051 | SYQCTAEISL | 1846 | HLA-C*14:02 |
| 2931 | AFP | ENSG00000081051 | SYQCTAEI | 1847 | HLA-C*14:02 |
| 2932 | AFP | ENSG00000081051 | TAEISLADL | 1848 | HLA-C*05:01 |
| 2933 | AFP | ENSG00000081051 | TECCKLTTL | 1849 | HLA-B*40:02 |
| 2934 | AFP | ENSG00000081051 | TEEQLEAVI | 1850 | HLA-B*40:01 |
| 2935 | AFP | ENSG00000081051 | TEEQLEAVI | 1850 | HLA-B*49:01 |
| 2936 | AFP | ENSG00000081051 | TEIQKLVLDV | 1851 | HLA-B*49:01 |
| 2937 | AFP | ENSG00000081051 | TEIQKLVL | 1852 | HLA-A*30:01 |
| 2938 | AFP | ENSG00000081051 | TEIQKLVL | 1852 | HLA-B*08:01 |
| 2939 | AFP | ENSG00000081051 | TEIQKLVL | 1852 | HLA-B*18:01 |
| 2940 | AFP | ENSG00000081051 | TEIQKLVL | 1852 | HLA-B*37:01 |
| 2941 | AFP | ENSG00000081051 | TEIQKLVL | 1852 | HLA-B*40:01 |
| 2942 | AFP | ENSG00000081051 | TEIQKLVL | 1852 | HLA-B*40:02 |
| 2943 | AFP | ENSG00000081051 | TEIQKLVL | 1852 | HLA-B*49:01 |
| 2944 | AFP | ENSG00000081051 | TENPLECQDK | 1853 | HLA-B*27:02 |
| 2945 | AFP | ENSG00000081051 | TFQAITVTKL | 1854 | HLA-A*23:01 |
| 2946 | AFP | ENSG00000081051 | TFQAITVTKL | 1854 | HLA-A*33:03 |
| 2947 | AFP | ENSG00000081051 | TFQAITVTKL | 1854 | HLA-C*14:02 |
| 2948 | AFP | ENSG00000081051 | TFQAITVTK | 1855 | HLA-A*03:02 |
| 2949 | AFP | ENSG00000081051 | TFQAITVTK | 1855 | HLA-A*23:01 |
| 2950 | AFP | ENSG00000081051 | TFQAITVTK | 1855 | HLA-A*33:01 |
| 2951 | AFP | ENSG00000081051 | TFQAITVTK | 1855 | HLA-A*33:03 |
| 2952 | AFP | ENSG00000081051 | TFQAITVTK | 1855 | HLA-C*14:02 |
| 2953 | AFP | ENSG00000081051 | TGDEQSSGCL | 1856 | HLA-C*05:01 |
| 2954 | AFP | ENSG00000081051 | TILLWAARY | 1857 | HLA-A*29:02 |
| 2955 | AFP | ENSG00000081051 | TLSNKITEC | 1858 | HLA-A*02:01 |
| 2956 | AFP | ENSG00000081051 | TLSNKITEC | 1858 | HLA-A*02:03 |
| 2957 | AFP | ENSG00000081051 | TLSNKITEC | 1858 | HLA-A*02:04 |
| 2958 | AFP | ENSG00000081051 | TLSNKITEC | 1858 | HLA-B*55:01 |
| 2959 | AFP | ENSG00000081051 | TMKQEFLINL | 1859 | HLA-A*02:03 |
| 2960 | AFP | ENSG00000081051 | TPASIPLFQV | 1860 | HLA-B*56:01 |
| 2961 | AFP | ENSG00000081051 | TPVNPGVGQC | 1861 | HLA-B*56:01 |
| 2962 | AFP | ENSG00000081051 | TPVNPGVGQ | 1862 | HLA-B*35:01 |
| 2963 | AFP | ENSG00000081051 | TPVNPGVGQ | 1862 | HLA-B*56:01 |
| 2964 | AFP | ENSG00000081051 | TRTFQAITVTK | 1863 | HLA-B*27:05 |
| 2965 | AFP | ENSG00000081051 | TRTFQAITV | 1864 | HLA-B*27:05 |
| 2966 | AFP | ENSG00000081051 | TRTFQAITV | 1864 | HLA-C*06:02 |
| 2967 | AFP | ENSG00000081051 | TSSELMAITRK | 1865 | HLA-A*11:01 |
| 2968 | AFP | ENSG00000081051 | TSSELMAITR | 1866 | HLA-A*11:01 |
| 2969 | AFP | ENSG00000081051 | TSSELMAITR | 1866 | HLA-A*31:01 |
| 2970 | AFP | ENSG00000081051 | TSSELMAITR | 1866 | HLA-A*33:01 |
| 2971 | AFP | ENSG00000081051 | TSSELMAITR | 1866 | HLA-A*33:03 |
| 2972 | AFP | ENSG00000081051 | TSSELMAITR | 1866 | HLA-A*68:01 |
| 2973 | AFP | ENSG00000081051 | TSSELMAITR | 1866 | HLA-A*68:02 |
| 2974 | AFP | ENSG00000081051 | TSSELMAITR | 1866 | HLA-B*27:02 |
| 2975 | AFP | ENSG00000081051 | TSSELMAITR | 1866 | HLA-B*57:01 |
| 2976 | AFP | ENSG00000081051 | TSSELMAITR | 1866 | HLA-C*07:06 |
| 2977 | AFP | ENSG00000081051 | TVTKLSQKF | 1867 | HLA-A*25:01 |
| 2978 | AFP | ENSG00000081051 | TVTKLSQKF | 1867 | HLA-A*26:01 |
| 2979 | AFP | ENSG00000081051 | TVTKLSQKF | 1867 | HLA-A*32:01 |
| 2980 | AFP | ENSG00000081051 | TVTKLSQKF | 1867 | HLA-B*35:01 |
| 2981 | AFP | ENSG00000081051 | TVTKLSQKF | 1867 | HLA-B*44:03 |
| 2982 | AFP | ENSG00000081051 | TVTKLSQKF | 1867 | HLA-B*58:01 |
| 2983 | AFP | ENSG00000081051 | TVTKLSQK | 1868 | HLA-A*03:02 |
| 2984 | AFP | ENSG00000081051 | TYKEVSKMVK | 1869 | HLA-A*31:01 |
| 2985 | AFP | ENSG00000081051 | TYKEVSKMVK | 1869 | HLA-A*33:01 |
| 2986 | AFP | ENSG00000081051 | TYKEVSKMVK | 1869 | HLA-A*33:03 |
| 2987 | AFP | ENSG00000081051 | TYKEVSKMVK | 1869 | HLA-C*04:01 |
| 2988 | AFP | ENSG00000081051 | TYKEVSKMVK | 1869 | HLA-C*06:02 |
| 2989 | AFP | ENSG00000081051 | TYKEVSKMV | 1870 | HLA-A*24:02 |
| 2990 | AFP | ENSG00000081051 | TYKEVSKMV | 1870 | HLA-C*06:02 |
| 2991 | AFP | ENSG00000081051 | TYKEVSKMV | 1870 | HLA-C*16:02 |
| 2992 | AFP | ENSG00000081051 | TYKEVSKM | 1871 | HLA-B*08:01 |
| 2993 | AFP | ENSG00000081051 | TYKEVSKM | 1871 | HLA-C*14:02 |
| 2994 | AFP | ENSG00000081051 | VAKGYQELL | 1872 | HLA-A*23:01 |
| 2995 | AFP | ENSG00000081051 | VAKGYQELL | 1872 | HLA-B*46:01 |
| 2996 | AFP | ENSG00000081051 | VAKGYQELL | 1872 | HLA-C*02:02 |
| 2997 | AFP | ENSG00000081051 | VAKGYQELL | 1872 | HLA-C*07:04 |
| 2998 | AFP | ENSG00000081051 | VAKGYQELL | 1872 | HLA-C*12:03 |
| 2999 | AFP | ENSG00000081051 | VAKGYQELL | 1872 | HLA-C*16:02 |
| 3000 | AFP | ENSG00000081051 | VAKGYQEL | 1873 | HLA-B*08:01 |
| 3001 | AFP | ENSG00000081051 | VAKGYQEL | 1873 | HLA-B*46:01 |
| 3002 | AFP | ENSG00000081051 | VAKGYQEL | 1873 | HLA-C*03:04 |
| 3003 | AFP | ENSG00000081051 | VAKGYQEL | 1873 | HLA-C*16:01 |
| 3004 | AFP | ENSG00000081051 | VECFQTKAATV | 1874 | HLA-B*49:01 |
| 3005 | AFP | ENSG00000081051 | VESIFLIF | 1875 | HLA-B*18:01 |
| 3006 | AFP | ENSG00000081051 | VESIFLIF | 1875 | HLA-B*37:01 |
| 3007 | AFP | ENSG00000081051 | VGQCCTSSY | 1876 | HLA-B*46:01 |
| 3008 | AFP | ENSG00000081051 | VGQCCTSSY | 1876 | HLA-C*14:02 |
| 3009 | AFP | ENSG00000081051 | VIADFSGLLEK | 1877 | HLA-A*03:01 |
| 3010 | AFP | ENSG00000081051 | VIADFSGLLEK | 1877 | HLA-A*03:02 |
| 3011 | AFP | ENSG00000081051 | VIADFSGLLEK | 1877 | HLA-A*11:01 |
| 3012 | AFP | ENSG00000081051 | VIADFSGLLEK | 1877 | HLA-A*31:01 |
| 3013 | AFP | ENSG00000081051 | VIADFSGLLEK | 1877 | HLA-A*33:01 |
| 3014 | AFP | ENSG00000081051 | VIADFSGLLEK | 1877 | HLA-B*27:02 |
| 3015 | AFP | ENSG00000081051 | VIADFSGLL | 1878 | HLA-A*02:03 |
| 3016 | AFP | ENSG00000081051 | VIADFSGLL | 1878 | HLA-A*03:01 |
| 3017 | AFP | ENSG00000081051 | VIADFSGLL | 1878 | HLA-A*23:01 |
| 3018 | AFP | ENSG00000081051 | VIADFSGLL | 1878 | HLA-A*24:02 |
| 3019 | AFP | ENSG00000081051 | VIADFSGLL | 1878 | HLA-A*26:01 |
| 3020 | AFP | ENSG00000081051 | VIADFSGLL | 1878 | HLA-A*68:02 |
| 3021 | AFP | ENSG00000081051 | VIADFSGLL | 1878 | HLA-B*13:02 |
| 3022 | AFP | ENSG00000081051 | VIADFSGLL | 1878 | HLA-B*27:05 |
| 3023 | AFP | ENSG00000081051 | VIADFSGLL | 1878 | HLA-C*03:03 |
| 3024 | AFP | ENSG00000081051 | VIADFSGLL | 1878 | HLA-C*03:04 |
| 3025 | AFP | ENSG00000081051 | VIADFSGLL | 1878 | HLA-C*07:04 |
| 3026 | AFP | ENSG00000081051 | VIADFSGL | 1879 | HLA-C*01:02 |
| 3027 | AFP | ENSG00000081051 | VIADFSGL | 1879 | HLA-C*05:01 |
| 3028 | AFP | ENSG00000081051 | VILRVAKGY | 1880 | HLA-A*29:02 |
| 3029 | AFP | ENSG00000081051 | VLDVAHVHEH | 1881 | HLA-A*01:01 |
| 3030 | AFP | ENSG00000081051 | VLDVAHVH | 1882 | HLA-C*04:01 |
| 3031 | AFP | ENSG00000081051 | VMKNFGTRTF | 1883 | HLA-A*24:02 |
| 3032 | AFP | ENSG00000081051 | VMKNFGTRTF | 1883 | HLA-A*32:01 |
| 3033 | AFP | ENSG00000081051 | VMKNFGTRTF | 1883 | HLA-B*46:01 |
| 3034 | AFP | ENSG00000081051 | VMKNFGTRTF | 1883 | HLA-B*57:01 |
| 3035 | AFP | ENSG00000081051 | VMKNFGTR | 1884 | HLA-A*31:01 |
| 3036 | AFP | ENSG00000081051 | VMKNFGTR | 1884 | HLA-A*33:01 |
| 3037 | AFP | ENSG00000081051 | VNFTEIQKL | 1885 | HLA-A*02:04 |
| 3038 | AFP | ENSG00000081051 | VNFTEIQKL | 1885 | HLA-A*23:01 |
| 3039 | AFP | ENSG00000081051 | VNFTEIQKL | 1885 | HLA-C*12:03 |
| 3040 | AFP | ENSG00000081051 | VPEPVTSCEAY | 1886 | HLA-A*01:01 |
| 3041 | AFP | ENSG00000081051 | VPEPVTSCEAY | 1886 | HLA-B*35:01 |
| 3042 | AFP | ENSG00000081051 | VPEPVTSCEAY | 1886 | HLA-B*55:01 |
| 3043 | AFP | ENSG00000081051 | VPEPVTSCEA | 1887 | HLA-B*56:01 |
| 3044 | AFP | ENSG00000081051 | VPEPVTSC | 1888 | HLA-B*56:01 |
| 3045 | AFP | ENSG00000081051 | VQEATYKEVSK | 1889 | HLA-A*03:02 |
| 3046 | AFP | ENSG00000081051 | VQEATYKEV | 1890 | HLA-B*13:02 |
| 3047 | AFP | ENSG00000081051 | VQEATYKEV | 1890 | HLA-B*40:02 |
| 3048 | AFP | ENSG00000081051 | VQEATYKEV | 1890 | HLA-C*05:01 |
| 3049 | AFP | ENSG00000081051 | VQEATYKEV | 1890 | HLA-C*06:02 |
| 3050 | AFP | ENSG00000081051 | VQEATYKEV | 1890 | HLA-C*16:02 |
| 3051 | AFP | ENSG00000081051 | VSKMVKDAL | 1891 | HLA-C*01:02 |
| 3052 | AFP | ENSG00000081051 | VTKELRESSL | 1892 | HLA-B*08:01 |
| 3053 | AFP | ENSG00000081051 | VTKELRESSL | 1892 | HLA-C*01:02 |
| 3054 | AFP | ENSG00000081051 | VTKLSQKF | 1893 | HLA-A*23:01 |
| 3055 | AFP | ENSG00000081051 | VTKLSQKF | 1893 | HLA-B*57:01 |
| 3056 | AFP | ENSG00000081051 | VVDETYVPPAF | 1894 | HLA-A*02:07 |
| 3057 | AFP | ENSG00000081051 | VVDETYVPPAF | 1894 | HLA-B*27:02 |
| 3058 | AFP | ENSG00000081051 | VVDETYVPPAF | 1894 | HLA-B*38:01 |
| 3059 | AFP | ENSG00000081051 | VVDETYVPPAF | 1894 | HLA-C*05:01 |
| 3060 | AFP | ENSG00000081051 | VVDETYVPP | 1895 | HLA-C*05:01 |
| 3061 | AFP | ENSG00000081051 | YAPTILLW | 1896 | HLA-B*51:01 |
| 3062 | AFP | ENSG00000081051 | YEEDRETFM | 1897 | HLA-A*30:01 |
| 3063 | AFP | ENSG00000081051 | YEEDRETFM | 1897 | HLA-B*37:01 |
| 3064 | AFP | ENSG00000081051 | YEEDRETFM | 1897 | HLA-B*40:01 |
| 3065 | AFP | ENSG00000081051 | YEEDRETFM | 1897 | HLA-B*40:02 |
| 3066 | AFP | ENSG00000081051 | YEEDRETFM | 1897 | HLA-B*44:02 |
| 3067 | AFP | ENSG00000081051 | YEEDRETFM | 1897 | HLA-B*49:01 |
| 3068 | AFP | ENSG00000081051 | YEEDRETFM | 1897 | HLA-C*05:01 |
| 3069 | AFP | ENSG00000081051 | YEEDRETFM | 1897 | HLA-C*16:04 |
| 3070 | AFP | ENSG00000081051 | YEEDRETF | 1898 | HLA-B*18:01 |
| 3071 | AFP | ENSG00000081051 | YEEDRETF | 1898 | HLA-B*37:01 |
| 3072 | AFP | ENSG00000081051 | YGIASILDSY | 1899 | HLA-A*01:01 |
| 3073 | AFP | ENSG00000081051 | YGIASILDSY | 1899 | HLA-A*26:01 |
| 3074 | AFP | ENSG00000081051 | YGIASILDSY | 1899 | HLA-A*29:02 |
| 3075 | AFP | ENSG00000081051 | YGIASILDSY | 1899 | HLA-A*30:02 |
| 3076 | AFP | ENSG00000081051 | YGIASILDSY | 1899 | HLA-B*15:01 |
| 3077 | AFP | ENSG00000081051 | YGIASILDSY | 1899 | HLA-B*27:02 |
| 3078 | AFP | ENSG00000081051 | YGIASILDSY | 1899 | HLA-B*35:01 |
| 3079 | AFP | ENSG00000081051 | YGIASILDSY | 1899 | HLA-B*46:01 |
| 3080 | AFP | ENSG00000081051 | YGIASILDSY | 1899 | HLA-C*02:02 |
| 3081 | AFP | ENSG00000081051 | YGIASILDSY | 1899 | HLA-C*16:04 |
| 3082 | AFP | ENSG00000081051 | YICSQQDTL | 1900 | HLA-B*35:03 |
| 3083 | AFP | ENSG00000081051 | YICSQQDTL | 1900 | HLA-B*38:01 |
| 3084 | AFP | ENSG00000081051 | YICSQQDTL | 1900 | HLA-B*39:01 |
| 3085 | AFP | ENSG00000081051 | YICSQQDTL | 1900 | HLA-B*40:01 |
| 3086 | AFP | ENSG00000081051 | YICSQQDTL | 1900 | HLA-C*02:02 |
| 3087 | AFP | ENSG00000081051 | YICSQQDTL | 1900 | HLA-C*03:03 |
| 3088 | AFP | ENSG00000081051 | YICSQQDTL | 1900 | HLA-C*03:04 |
| 3089 | AFP | ENSG00000081051 | YICSQQDTL | 1900 | HLA-C*07:04 |
| 3090 | AFP | ENSG00000081051 | YIQESQALAKR | 1901 | HLA-A*03:02 |
| 3091 | AFP | ENSG00000081051 | YIQESQALAKR | 1901 | HLA-A*11:01 |
| 3092 | AFP | ENSG00000081051 | YIQESQALAKR | 1901 | HLA-A*31:01 |
| 3093 | AFP | ENSG00000081051 | YIQESQALAKR | 1901 | HLA-A*33:01 |
| 3094 | AFP | ENSG00000081051 | YIQESQALAKR | 1901 | HLA-A*33:03 |
| 3095 | AFP | ENSG00000081051 | YIQESQALAKR | 1901 | HLA-A*68:01 |
| 3096 | AFP | ENSG00000081051 | YIQESQALAKR | 1901 | HLA-B*27:02 |
| 3097 | AFP | ENSG00000081051 | YIQESQALAKR | 1901 | HLA-C*07:06 |
| 3098 | AFP | ENSG00000081051 | YIQESQALAK | 1902 | HLA-A*01:01 |
| 3099 | AFP | ENSG00000081051 | YIQESQALAK | 1902 | HLA-A*03:01 |
| 3100 | AFP | ENSG00000081051 | YIQESQALAK | 1902 | HLA-A*03:02 |
| 3101 | AFP | ENSG00000081051 | YIQESQALAK | 1902 | HLA-A*11:01 |
| 3102 | AFP | ENSG00000081051 | YIQESQALAK | 1902 | HLA-A*68:01 |
| 3103 | AFP | ENSG00000081051 | YIQESQALAK | 1902 | HLA-B*27:02 |
| 3104 | AFP | ENSG00000081051 | YIQESQALAK | 1902 | HLA-C*07:06 |
| 3105 | AFP | ENSG00000081051 | YIQESQALA | 1903 | HLA-A*02:01 |
| 3106 | AFP | ENSG00000081051 | YIQESQAL | 1904 | HLA-A*30:01 |
| 3107 | AFP | ENSG00000081051 | YIQESQAL | 1904 | HLA-B*08:01 |
| 3108 | AFP | ENSG00000081051 | YIQESQAL | 1904 | HLA-B*15:01 |
| 3109 | AFP | ENSG00000081051 | YIQESQAL | 1904 | HLA-B*27:05 |
| 3110 | AFP | ENSG00000081051 | YIQESQAL | 1904 | HLA-B*35:03 |
| 3111 | AFP | ENSG00000081051 | YIQESQAL | 1904 | HLA-B*37:01 |
| 3112 | AFP | ENSG00000081051 | YIQESQAL | 1904 | HLA-B*39:01 |
| 3113 | AFP | ENSG00000081051 | YIQESQAL | 1904 | HLA-B*40:01 |
| 3114 | AFP | ENSG00000081051 | YIQESQAL | 1904 | HLA-B*40:02 |
| 3115 | AFP | ENSG00000081051 | YIQESQAL | 1904 | HLA-B*46:01 |
| 3116 | AFP | ENSG00000081051 | YIQESQAL | 1904 | HLA-C*01:02 |
| 3117 | AFP | ENSG00000081051 | YIQESQAL | 1904 | HLA-C*03:03 |
| 3118 | AFP | ENSG00000081051 | YIQESQAL | 1904 | HLA-C*03:04 |
| 3119 | AFP | ENSG00000081051 | YIQESQAL | 1904 | HLA-C*05:01 |
| 3120 | AFP | ENSG00000081051 | YIQESQAL | 1904 | HLA-C*07:04 |
| 3121 | AFP | ENSG00000081051 | YIQESQAL | 1904 | HLA-C*14:02 |
| 3122 | AFP | ENSG00000081051 | YIQESQAL | 1904 | HLA-C*16:01 |
| 3123 | AFP | ENSG00000081051 | YIQESQAL | 1904 | HLA-C*16:02 |
| 3124 | AFP | ENSG00000081051 | YLQNAFLVAY | 1905 | HLA-A*01:01 |
| 3125 | AFP | ENSG00000081051 | YLQNAFLVAY | 1905 | HLA-A*29:02 |
| 3126 | AFP | ENSG00000081051 | YLQNAFLVAY | 1905 | HLA-A*30:02 |
| 3127 | AFP | ENSG00000081051 | YLQNAFLVAY | 1905 | HLA-B*15:01 |
| 3128 | AFP | ENSG00000081051 | YLQNAFLVAY | 1905 | HLA-B*46:01 |
| 3129 | AFP | ENSG00000081051 | YLQNAFLVA | 1906 | HLA-A*02:01 |
| 3130 | AFP | ENSG00000081051 | YLQNAFLVA | 1906 | HLA-A*02:04 |
| 3131 | AFP | ENSG00000081051 | YLQNAFLVA | 1906 | HLA-B*54:01 |
| 3132 | AFP | ENSG00000081051 | YLQNAFLV | 1907 | HLA-A*02:04 |
| 3133 | AFP | ENSG00000081051 | YLQNAFLV | 1907 | HLA-B*13:02 |
| 3134 | AFP | ENSG00000081051 | YQCTAEISL | 1908 | HLA-B*13:02 |
| 3135 | AFP | ENSG00000081051 | YQCTAEISL | 1908 | HLA-B*27:05 |
| 3136 | AFP | ENSG00000081051 | YQCTAEISL | 1908 | HLA-B*38:01 |
| 3137 | AFP | ENSG00000081051 | YQCTAEISL | 1908 | HLA-B*39:01 |
| 3138 | AFP | ENSG00000081051 | YQCTAEISL | 1908 | HLA-B*40:01 |
| 3139 | AFP | ENSG00000081051 | YQCTAEISL | 1908 | HLA-C*03:03 |
| 3140 | AFP | ENSG00000081051 | YQCTAEISL | 1908 | HLA-C*07:04 |
| 3141 | AFP | ENSG00000081051 | YTKKAPQL | 1909 | HLA-B*07:02 |
| 3142 | AFP | ENSG00000081051 | YTKKAPQL | 1909 | HLA-B*08:01 |
| 3143 | AFP | ENSG00000081051 | YTKKAPQL | 1909 | HLA-B*40:02 |
| 3144 | AFP | ENSG00000081051 | YTKKAPQL | 1909 | HLA-B*51:01 |
| 3145 | AFP | ENSG00000081051 | YTKKAPQL | 1909 | HLA-B*58:01 |
| 3146 | AFP | ENSG00000081051 | YTKKAPQL | 1909 | HLA-C*03:03 |
| 3147 | AFP | ENSG00000081051 | YTKKAPQL | 1909 | HLA-C*03:04 |
| 3148 | AFP | ENSG00000081051 | YTKKAPQL | 1909 | HLA-C*06:02 |
| 3149 | AFP | ENSG00000081051 | YTKKAPQL | 1909 | HLA-C*07:01 |
| 3150 | AFP | ENSG00000081051 | YTKKAPQL | 1909 | HLA-C*12:03 |
| 3151 | AFP | ENSG00000081051 | YTKKAPQL | 1909 | HLA-C*16:01 |
| 3152 | AFP | ENSG00000081051 | YTKKAPQL | 1909 | HLA-C*16:02 |
| 3153 | AFP | ENSG00000081051 | YYLQNAFLVAY | 1910 | HLA-A*29:02 |
| 3154 | AFP | ENSG00000081051 | YYLQNAFLV | 1911 | HLA-A*23:01 |
| 3155 | AFP | ENSG00000081051 | YYLQNAFLV | 1911 | HLA-A*24:02 |
| 3156 | AFP | ENSG00000081051 | YYLQNAFLV | 1911 | HLA-A*29:02 |
| 3157 | AFP | ENSG00000081051 | YYLQNAFL | 1912 | HLA-A*23:01 |
| 3158 | MART1 | ENSG00000120215 | AAGIGILTV | 1913 | HLA-A*02:03 |
| 3159 | MART1 | ENSG00000120215 | AAGIGILTV | 1913 | HLA-B*13:02 |
| 3160 | MART1 | ENSG00000120215 | AAGIGILTV | 1913 | HLA-B*49:01 |
| 3161 | MART1 | ENSG00000120215 | AAGIGILTV | 1913 | HLA-B*51:01 |
| 3162 | MART1 | ENSG00000120215 | AAGIGILTV | 1913 | HLA-C*I2:03 |
| 3163 | MART1 | ENSG00000120215 | AEEAAGIGIL | 1914 | HLA-A*30:01 |
| 3164 | MART1 | ENSG00000120215 | AEEAAGIGIL | 1914 | HLA-B*40:01 |
| 3165 | MART1 | ENSG00000120215 | AEEAAGIGIL | 1914 | HLA-B*40:02 |
| 3166 | MART1 | ENSG00000120215 | AEEAAGIGIL | 1914 | HLA-B*44:02 |
| 3167 | MART1 | ENSG00000120215 | AEEAAGIGIL | 1914 | HLA-B*49:01 |
| 3168 | MART1 | ENSG00000120215 | AEEAAGIGI | 1915 | HLA-A*30:01 |
| 3169 | MART1 | ENSG00000120215 | AEEAAGIGI | 1915 | HLA-B*40:01 |
| 3170 | MART1 | ENSG00000120215 | AEEAAGIGI | 1915 | HLA-B*44:02 |
| 3171 | MART1 | ENSG00000120215 | AEEAAGIGI | 1915 | HLA-B*44:03 |
| 3172 | MART1 | ENSG00000120215 | AEEAAGIGI | 1915 | HLA-B*49:01 |
| 3173 | MART1 | ENSG00000120215 | AEQSPPPYSP | 1916 | HLA-B*27:05 |
| 3174 | MART1 | ENSG00000120215 | AEQSPPPYSP | 1916 | HLA-B*40:02 |
| 3175 | MART1 | ENSG00000120215 | AEQSPPPYSP | 1916 | HLA-B*44:02 |
| 3176 | MART1 | ENSG00000120215 | AEQSPPPY | 1917 | HLA-A*30:02 |
| 3177 | MART1 | ENSG00000120215 | AEQSPPPY | 1917 | HLA-B*I 8:01 |
| 3178 | MART1 | ENSG00000120215 | AEQSPPPY | 1917 | HLA-B*37:01 |
| 3179 | MART1 | ENSG00000120215 | AEQSPPPY | 1917 | HLA-B*44:03 |
| 3180 | MART1 | ENSG00000120215 | AGIGILTVI | 1918 | HLA-A*23:01 |
| 3181 | MART1 | ENSG00000120215 | AGIGILTVI | 1918 | HLA-B*13:02 |
| 3182 | MART1 | ENSG00000120215 | AGIGILTVI | 1918 | HLA-B*49:01 |
| 3183 | MART1 | ENSG00000120215 | AGIGILTVI | 1918 | HLA-C*02:02 |
| 3184 | MART1 | ENSG00000120215 | ALMDKSLHVG | 1919 | HLA-A*02:01 |
| 3185 | MART1 | ENSG00000120215 | ALMDKSLHV | 1920 | HLA-A*02:01 |
| 3186 | MART1 | ENSG00000120215 | ALMDKSLHV | 1920 | HLA-A*02:03 |
| 3187 | MART1 | ENSG00000120215 | ALMDKSLHV | 1920 | HLA-A*02:04 |
| 3188 | MART1 | ENSG00000120215 | ALMDKSLHV | 1920 | HLA-A*02:07 |
| 3189 | MART1 | ENSG00000120215 | ALMDKSLHV | 1920 | HLA-B*08:01 |
| 3190 | MART1 | ENSG00000120215 | ALMDKSLHV | 1920 | HLA-B*13:02 |
| 3191 | MART1 | ENSG00000120215 | ALMDKSLHV | 1920 | HLA-B*55:01 |
| 3192 | MART1 | ENSG00000120215 | APPAYEKLSA | 1921 | HLA-B*54:01 |
| 3193 | MART1 | ENSG00000120215 | APPAYEKLSA | 1921 | HLA-B*56:01 |
| 3194 | MART1 | ENSG00000120215 | CPQEGFDHR | 1922 | HLA-A*33:03 |
| 3195 | MART1 | ENSG00000120215 | DAHFIYGYPK | 1923 | HLA-A*33:01 |
| 3196 | MART1 | ENSG00000120215 | DAHFIYGY | 1924 | HLA-B*18:01 |
| 3197 | MART1 | ENSG00000120215 | DAHFIYGY | 1924 | HLA-B*35:01 |
| 3198 | MART1 | ENSG00000120215 | DAHFIYGY | 1924 | HLA-B*51:01 |
| 3199 | MART1 | ENSG00000120215 | DHRDSKVSL | 1925 | HLA-B*08:01 |
| 3200 | MART1 | ENSG00000120215 | DHRDSKVSL | 1925 | HLA-B*38:01 |
| 3201 | MART1 | ENSG00000120215 | DHRDSKVSL | 1925 | HLA-B*39:01 |
| 3202 | MART1 | ENSG00000120215 | DSKVSLQEK | 1926 | HLA-A*33:01 |
| 3203 | MART1 | ENSG00000120215 | DSKVSLQEK | 1926 | HLA-A*33:03 |
| 3204 | MART1 | ENSG00000120215 | DSKVSLQEK | 1926 | HLA-A*68:01 |
| 3205 | MART1 | ENSG00000120215 | DSKVSLQEK | 1926 | HLA-C*07:06 |
| 3206 | MART1 | ENSG00000120215 | EAAGIGILTVI | 1927 | HLA-A*25:01 |
| 3207 | MART1 | ENSG00000120215 | EAAGIGILTVI | 1927 | HLA-A*26:01 |
| 3208 | MART1 | ENSG00000120215 | EAAGIGILTVI | 1927 | HLA-A*68:01 |
| 3209 | MART1 | ENSG00000120215 | EAAGIGILTVI | 1927 | HLA-A*68:02 |
| 3210 | MART1 | ENSG00000120215 | EAAGIGILTV | 1928 | HLA-A*25:01 |
| 3211 | MART1 | ENSG00000120215 | EAAGIGILTV | 1928 | HLA-A*26:01 |
| 3212 | MART1 | ENSG00000120215 | EAAGIGILTV | 1928 | HLA-A*68:01 |
| 3213 | MART1 | ENSG00000120215 | EAAGIGILTV | 1928 | HLA-A*68:02 |
| 3214 | MART1 | ENSG00000120215 | EAAGIGILTV | 1928 | HLA-B*51:01 |
| 3215 | MART1 | ENSG00000120215 | EAAGIGILTV | 1928 | HLA-B*54:01 |
| 3216 | MART1 | ENSG00000120215 | EAAGIGILTV | 1928 | HLA-C*07:06 |
| 3217 | MART1 | ENSG00000120215 | EAAGIGILT | 1929 | HLA-A*33:03 |
| 3218 | MART1 | ENSG00000120215 | EAAGIGILT | 1929 | HLA-A*68:01 |
| 3219 | MART1 | ENSG00000120215 | EAAGIGILT | 1929 | HLA-A*68:02 |
| 3220 | MART1 | ENSG00000120215 | EAAGIGILT | 1929 | HLA-C*07:06 |
| 3221 | MART1 | ENSG00000120215 | EDAHFIYGY | 1930 | HLA-A*26:01 |
| 3222 | MART1 | ENSG00000120215 | EEAAGIGILTV | 1931 | HLA-A*30:01 |
| 3223 | MART1 | ENSG00000120215 | EEAAGIGILTV | 1931 | HLA-A*68:02 |
| 3224 | MART1 | ENSG00000120215 | EEAAGIGILTV | 1931 | HLA-B*44:02 |
| 3225 | MART1 | ENSG00000120215 | EEAAGIGILTV | 1931 | HLA-B*44:03 |
| 3226 | MART1 | ENSG00000120215 | EEAAGIGILTV | 1931 | HLA-B*49:01 |
| 3227 | MART1 | ENSG00000120215 | EEAAGIGIL | 1932 | HLA-A*30:01 |
| 3228 | MART1 | ENSG00000120215 | EEAAGIGIL | 1932 | HLA-B*38:01 |
| 3229 | MART1 | ENSG00000120215 | EEAAGIGIL | 1932 | HLA-B*40:01 |
| 3230 | MART1 | ENSG00000120215 | EEAAGIGIL | 1932 | HLA-B*44:02 |
| 3231 | MART1 | ENSG00000120215 | EEAAGIGIL | 1932 | HLA-B*44:03 |
| 3232 | MART1 | ENSG00000120215 | EEAAGIGI | 1933 | HLA-B*44:02 |
| 3233 | MART1 | ENSG00000120215 | EEAAGIGI | 1933 | HLA-B*44:03 |
| 3234 | MART1 | ENSG00000120215 | EEAAGIGI | 1933 | HLA-B*49:01 |
| 3235 | MART1 | ENSG00000120215 | EPVVPNAPPAY | 1934 | HLA-A*01:01 |
| 3236 | MART1 | ENSG00000120215 | EPVVPNAPPAY | 1934 | HLA-A*26:01 |
| 3237 | MART1 | ENSG00000120215 | EPVVPNAPPAY | 1934 | HLA-A*30:02 |
| 3238 | MART1 | ENSG00000120215 | EPVVPNAPPAY | 1934 | HLA-B*35:01 |
| 3239 | MART1 | ENSG00000120215 | EPVVPNAPPA | 1935 | HLA-B*54:01 |
| 3240 | MART1 | ENSG00000120215 | EQSPPPYSP | 1936 | HLA-B*13:02 |
| 3241 | MART1 | ENSG00000120215 | EQSPPPYSP | 1936 | HLA-B*27:05 |
| 3242 | MART1 | ENSG00000120215 | EQSPPPYSP | 1936 | HLA-B*38:01 |
| 3243 | MART1 | ENSG00000120215 | EQSPPPYSP | 1936 | HLA-B*39:01 |
| 3244 | MART1 | ENSG00000120215 | EQSPPPYSP | 1936 | HLA-B*40:02 |
| 3245 | MART1 | ENSG00000120215 | EQSPPPYSP | 1936 | HLA-C*06:02 |
| 3246 | MART1 | ENSG00000120215 | GILTVILGV | 1937 | HLA-A*02:01 |
| 3247 | MART1 | ENSG00000120215 | GILTVILGV | 1937 | HLA-A*02:03 |
| 3248 | MART1 | ENSG00000120215 | GILTVILGV | 1937 | HLA-A*02:04 |
| 3249 | MART1 | ENSG00000120215 | GILTVILGV | 1937 | HLA-A*02:07 |
| 3250 | MART1 | ENSG00000120215 | GILTVILGV | 1937 | HLA-B*13:02 |
| 3251 | MART1 | ENSG00000120215 | GTQCALTRR | 1938 | HLA-A*31:01 |
| 3252 | MART1 | ENSG00000120215 | HSYTTAEEA | 1939 | HLA-B*54:01 |
| 3253 | MART1 | ENSG00000120215 | HVGTQCALTR | 1940 | HLA-A*68:01 |
| 3254 | MART1 | ENSG00000120215 | HVGTQCALTR | 1940 | HLA-C*07:06 |
| 3255 | MART1 | ENSG00000120215 | HVGTQCAL | 1941 | HLA-B*08:01 |
| 3256 | MART1 | ENSG00000120215 | HVGTQCAL | 1941 | HLA-C*01:02 |
| 3257 | MART1 | ENSG00000120215 | HVGTQCAL | 1941 | HLA-C*07:04 |
| 3258 | MART1 | ENSG00000120215 | ILTVILGV | 1942 | HLA-B*13:02 |
| 3259 | MART1 | ENSG00000120215 | KLSAEQSPPPY | 1943 | HLA-A*03:02 |
| 3260 | MART1 | ENSG00000120215 | KLSAEQSPPPY | 1943 | HLA-A*30:02 |
| 3261 | MART1 | ENSG00000120215 | KSLHVGTQC | 1944 | HLA-B*58:01 |
| 3262 | MART1 | ENSG00000120215 | LHVGTQCAL | 1945 | HLA-B*38:01 |
| 3263 | MART1 | ENSG00000120215 | LHVGTQCAL | 1945 | HLA-B*39:01 |
| 3264 | MART1 | ENSG00000120215 | LSAEQSPPPY | 1946 | HLA-A*01:01 |
| 3265 | MART1 | ENSG00000120215 | LSAEQSPPPY | 1946 | HLA-A*26:01 |
| 3266 | MART1 | ENSG00000120215 | LSAEQSPPPY | 1946 | HLA-A*30:02 |
| 3267 | MART1 | ENSG00000120215 | MPREDAHFIY | 1947 | HLA-B*35:01 |
| 3268 | MART1 | ENSG00000120215 | MPREDAHFI | 1948 | HLA-B*51:01 |
| 3269 | MART1 | ENSG00000120215 | NAPPAYEKL | 1949 | HLA-A*02:07 |
| 3270 | MART1 | ENSG00000120215 | NAPPAYEKL | 1949 | HLA-B*35:03 |
| 3271 | MART1 | ENSG00000120215 | NAPPAYEKL | 1949 | HLA-B*51:01 |
| 3272 | MART1 | ENSG00000120215 | NAPPAYEKL | 1949 | HLA-C*01:02 |
| 3273 | MART1 | ENSG00000120215 | NAPPAYEKL | 1949 | HLA-C*05:01 |
| 3274 | MART1 | ENSG00000120215 | PVVPNAPPAY | 1950 | HLA-A*26:01 |
| 3275 | MART1 | ENSG00000120215 | PVVPNAPPAY | 1950 | HLA-A*30:02 |
| 3276 | MART1 | ENSG00000120215 | QEKNCEPVV | 1951 | HLA-B*40:02 |
| 3277 | MART1 | ENSG00000120215 | QEKNCEPVV | 1951 | HLA-B*49:01 |
| 3278 | MART1 | ENSG00000120215 | REDAHFIYGY | 1952 | HLA-B*44:02 |
| 3279 | MART1 | ENSG00000120215 | SAEQSPPPY | 1953 | HLA-A*01:01 |
| 3280 | MART1 | ENSG00000120215 | SAEQSPPPY | 1953 | HLA-A*30:02 |
| 3281 | MART1 | ENSG00000120215 | SAEQSPPPY | 1953 | HLA-A*32:01 |
| 3282 | MART1 | ENSG00000120215 | SAEQSPPPY | 1953 | HLA-B*15:03 |
| 3283 | MART1 | ENSG00000120215 | SAEQSPPPY | 1953 | HLA-B*35:01 |
| 3284 | MART1 | ENSG00000120215 | SAEQSPPPY | 1953 | HLA-B*35:03 |
| 3285 | MART1 | ENSG00000120215 | SAEQSPPPY | 1953 | HLA-B*39:01 |
| 3286 | MART1 | ENSG00000120215 | SAEQSPPPY | 1953 | HLA-B*46:01 |
| 3287 | MART1 | ENSG00000120215 | SAEQSPPPY | 1953 | HLA-B*55:01 |
| 3288 | MART1 | ENSG00000120215 | SAEQSPPPY | 1953 | HLA-B*58:01 |
| 3289 | MART1 | ENSG00000120215 | SAEQSPPPY | 1953 | HLA-C*01:02 |
| 3290 | MART1 | ENSG00000120215 | SAEQSPPPY | 1953 | HLA-C*02:02 |
| 3291 | MART1 | ENSG00000120215 | SAEQSPPPY | 1953 | HLA-C*03:03 |
| 3292 | MART1 | ENSG00000120215 | SAEQSPPPY | 1953 | HLA-C*03:04 |
| 3293 | MART1 | ENSG00000120215 | SAEQSPPPY | 1953 | HLA-C*04:01 |
| 3294 | MART1 | ENSG00000120215 | SAEQSPPPY | 1953 | HLA-C*05:01 |
| 3295 | MART1 | ENSG00000120215 | SAEQSPPPY | 1953 | HLA-C*07:04 |
| 3296 | MART1 | ENSG00000120215 | SAEQSPPPY | 1953 | HLA-C*07:06 |
| 3297 | MART1 | ENSG00000120215 | SAEQSPPPY | 1953 | HLA-C*12:03 |
| 3298 | MART1 | ENSG00000120215 | SAEQSPPPY | 1953 | HLA-C*16:02 |
| 3299 | MART1 | ENSG00000120215 | SYTTAEEAA | 1954 | HLA-C*14:02 |
| 3300 | MART1 | ENSG00000120215 | SYTTAEEA | 1955 | HLA-C*14:02 |
| 3301 | MART1 | ENSG00000120215 | TAEEAAGIGIL | 1956 | HLA-B*35:03 |
| 3302 | MART1 | ENSG00000120215 | TAEEAAGIGIL | 1956 | HLA-C*05:01 |
| 3303 | MART1 | ENSG00000120215 | TAEEAAGIGI | 1957 | HLA-C*05:01 |
| 3304 | MART1 | ENSG00000120215 | TAEEAAGI | 1958 | HLA-C*05:01 |
| 3305 | MART1 | ENSG00000120215 | TTAEEAAGIGI | 1959 | HLA-A*26:01 |
| 3306 | MART1 | ENSG00000120215 | TTAEEAAGIGI | 1959 | HLA-A*68:01 |
| 3307 | MART1 | ENSG00000120215 | TTAEEAAGIGI | 1959 | HLA-A*68:02 |
| 3308 | MART1 | ENSG00000120215 | VILGVLLLI | 1960 | HLA-A*02:04 |
| 3309 | MART1 | ENSG00000120215 | VILGVLLLI | 1960 | HLA-A*23:01 |
| 3310 | MART1 | ENSG00000120215 | VPNAPPAYEKL | 1961 | HLA-B*07:02 |
| 3311 | MART1 | ENSG00000120215 | VPNAPPAYEKL | 1961 | HLA-B*35:03 |
| 3312 | MART1 | ENSG00000120215 | VPNAPPAYEK | 1962 | HLA-C*07:06 |
| 3313 | MART1 | ENSG00000120215 | VPNAPPAY | 1963 | HLA-B*35:01 |
| 3314 | MART1 | ENSG00000120215 | VVPNAPPAYEK | 1964 | HLA-A*03:01 |
| 3315 | MART1 | ENSG00000120215 | VVPNAPPAYEK | 1964 | HLA-A*03:02 |
| 3316 | MART1 | ENSG00000120215 | VVPNAPPAYEK | 1964 | HLA-A*11:01 |
| 3317 | MART1 | ENSG00000120215 | VVPNAPPAY | 1965 | HLA-A*25:01 |
| 3318 | MART1 | ENSG00000120215 | VVPNAPPAY | 1965 | HLA-A*26:01 |
| 3319 | MART1 | ENSG00000120215 | VVPNAPPAY | 1965 | HLA-A*29:02 |
| 3320 | MART1 | ENSG00000120215 | VVPNAPPAY | 1965 | HLA-A*30:02 |
| 3321 | MART1 | ENSG00000120215 | VVPNAPPAY | 1965 | HLA-A*32:01 |
| 3322 | MART1 | ENSG00000120215 | VVPNAPPAY | 1965 | HLA-B*15:01 |
| 3323 | MART1 | ENSG00000120215 | VVPNAPPAY | 1965 | HLA-B*46:01 |
| 3324 | MART1 | ENSG00000120215 | VVPNAPPAY | 1965 | HLA-C*01:02 |
| 3325 | MART1 | ENSG00000120215 | VVPNAPPAY | 1965 | HLA-C*07:04 |
| 3326 | MART1 | ENSG00000120215 | YRALMDKSL | 1966 | HLA-B*27:05 |
| 3327 | MART1 | ENSG00000120215 | YRALMDKSL | 1966 | HLA-C*06:02 |
| 3328 | MART1 | ENSG00000120215 | YRALMDKSL | 1966 | HLA-C*07:02 |
| 3329 | MAGEA10 | ENSG00000124260 | ACSSPSVVASL | 1967 | HLA-A*30:01 |
| 3330 | MAGEA10 | ENSG00000124260 | AEILESVIRNY | 64 | HLA-A*30:01 |
| 3331 | MAGEA10 | ENSG00000124260 | AEILESVIRNY | 64 | HLA-A*30:02 |
| 3332 | MAGEA10 | ENSG00000124260 | AEILESVIRNY | 64 | HLA-B*27:02 |
| 3333 | MAGEA10 | ENSG00000124260 | AEILESVIRNY | 64 | HLA-B*44:02 |
| 3334 | MAGEA10 | ENSG00000124260 | AEILESVIRNY | 64 | HLA-B*44:03 |
| 3335 | MAGEA10 | ENSG00000124260 | AEILESVIRNY | 64 | HLA-B*57:01 |
| 3336 | MAGEA10 | ENSG00000124260 | AEILESVIRNY | 64 | HLA-C*16:04 |
| 3337 | MAGEA10 | ENSG00000124260 | AEILESVIRN | 1968 | HLA-B*44:02 |
| 3338 | MAGEA10 | ENSG00000124260 | AEILESVI | 1969 | HLA-A*30:01 |
| 3339 | MAGEA10 | ENSG00000124260 | AEILESVI | 1969 | HLA-B*40:01 |
| 3340 | MAGEA10 | ENSG00000124260 | AEILESVI | 1969 | HLA-B*44:02 |
| 3341 | MAGEA10 | ENSG00000124260 | AEILESVI | 1969 | HLA-B*44:03 |
| 3342 | MAGEA10 | ENSG00000124260 | AEILESVI | 1969 | HLA-B*49:01 |
| 3343 | MAGEA10 | ENSG00000124260 | AEIRKMSLL | 1970 | HLA-B*37:01 |
| 3344 | MAGEA10 | ENSG00000124260 | AEIRKMSLL | 1970 | HLA-B*40:01 |
| 3345 | MAGEA10 | ENSG00000124260 | AEIRKMSLL | 1970 | HLA-B*40:02 |
| 3346 | MAGEA10 | ENSG00000124260 | AEIRKMSLL | 1970 | HLA-B*44:02 |
| 3347 | MAGEA10 | ENSG00000124260 | AEIRKMSLL | 1970 | HLA-B*44:03 |
| 3348 | MAGEA10 | ENSG00000124260 | AEIRKMSLL | 1970 | HLA-B*49:01 |
| 3349 | MAGEA10 | ENSG00000124260 | AEIRKMSL | 1971 | HLA-B*37:01 |
| 3350 | MAGEA10 | ENSG00000124260 | AEIRKMSL | 1971 | HLA-B*40:02 |
| 3351 | MAGEA10 | ENSG00000124260 | AKVNGSDPRSF | 1972 | HLA-B*15:03 |
| 3352 | MAGEA10 | ENSG00000124260 | ALNMMGLY | 1973 | HLA-A*30:02 |
| 3353 | MAGEA10 | ENSG00000124260 | AMASASSSA | 1974 | HLA-A*02:03 |
| 3354 | MAGEA10 | ENSG00000124260 | AMASASSSA | 1974 | HLA-A*32:01 |
| 3355 | MAGEA10 | ENSG00000124260 | AMASASSSA | 1974 | HLA-B*55:01 |
| 3356 | MAGEA10 | ENSG00000124260 | AMASASSSA | 1974 | HLA-C*01:02 |
| 3357 | MAGEA10 | ENSG00000124260 | AMASASSSA | 1974 | HLA-C*14:02 |
| 3358 | MAGEA10 | ENSG00000124260 | APLAVEEDA | 1975 | HLA-B*56:01 |
| 3359 | MAGEA10 | ENSG00000124260 | AQAPLAVEE | 1976 | HLA-B*27:05 |
| 3360 | MAGEA10 | ENSG00000124260 | AQIACSSPSV | 1977 | HLA-B*13:02 |
| 3361 | MAGEA10 | ENSG00000124260 | ASASSSATGSF | 1978 | HLA-A*25:01 |
| 3362 | MAGEA10 | ENSG00000124260 | ASASSSATGSF | 1978 | HLA-A*26:01 |
| 3363 | MAGEA10 | ENSG00000124260 | ASASSSATGSF | 1978 | HLA-A*30:02 |
| 3364 | MAGEA10 | ENSG00000124260 | ASASSSATGSF | 1978 | HLA-A*32:01 |
| 3365 | MAGEA10 | ENSG00000124260 | ASASSSATGSF | 1978 | HLA-B*15:01 |
| 3366 | MAGEA10 | ENSG00000124260 | ASASSSATGSF | 1978 | HLA-B*46:01 |
| 3367 | MAGEA10 | ENSG00000124260 | ASASSSATGSF | 1978 | HLA-B*58:01 |
| 3368 | MAGEA10 | ENSG00000124260 | ASASSSATGSF | 1978 | HLA-C*16:04 |
| 3369 | MAGEA10 | ENSG00000124260 | ASSSATGSFSY | 1979 | HLA-A*01:01 |
| 3370 | MAGEA10 | ENSG00000124260 | ASSSATGSFSY | 1979 | HLA-A*03:02 |
| 3371 | MAGEA10 | ENSG00000124260 | ASSSATGSFSY | 1979 | HLA-A*11:01 |
| 3372 | MAGEA10 | ENSG00000124260 | ASSSATGSFSY | 1979 | HLA-A*26:01 |
| 3373 | MAGEA10 | ENSG00000124260 | ASSSATGSFSY | 1979 | HLA-A*29:02 |
| 3374 | MAGEA10 | ENSG00000124260 | ASSSATGSFSY | 1979 | HLA-A*30:02 |
| 3375 | MAGEA10 | ENSG00000124260 | ASSSATGSFSY | 1979 | HLA-A*32:01 |
| 3376 | MAGEA10 | ENSG00000124260 | ASSSATGSFSY | 1979 | HLA-B*58:01 |
| 3377 | MAGEA10 | ENSG00000124260 | ASSSATGSFSY | 1979 | HLA-C*16:02 |
| 3378 | MAGEA10 | ENSG00000124260 | ASSSATGSFSY | 1979 | HLA-C*16:04 |
| 3379 | MAGEA10 | ENSG00000124260 | ASSSATGSF | 1980 | HLA-A*25:01 |
| 3380 | MAGEA10 | ENSG00000124260 | ASSSATGSF | 1980 | HLA-A*26:01 |
| 3381 | MAGEA10 | ENSG00000124260 | ASSSATGSF | 1980 | HLA-A*30:02 |
| 3382 | MAGEA10 | ENSG00000124260 | ASSSATGSF | 1980 | HLA-A*32:01 |
| 3383 | MAGEA10 | ENSG00000124260 | ASSSATGSF | 1980 | HLA-B*15:01 |
| 3384 | MAGEA10 | ENSG00000124260 | ASSSATGSF | 1980 | HLA-B*15:03 |
| 3385 | MAGEA10 | ENSG00000124260 | ASSSATGSF | 1980 | HLA-B*37:01 |
| 3386 | MAGEA10 | ENSG00000124260 | ASSSATGSF | 1980 | HLA-B*46:01 |
| 3387 | MAGEA10 | ENSG00000124260 | ASSSATGSF | 1980 | HLA-B*58:01 |
| 3388 | MAGEA10 | ENSG00000124260 | ASSSATGSF | 1980 | HLA-C*01:02 |
| 3389 | MAGEA10 | ENSG00000124260 | ASSSATGSF | 1980 | HLA-C*02:02 |
| 3390 | MAGEA10 | ENSG00000124260 | ASSSATGSF | 1980 | HLA-C*03:03 |
| 3391 | MAGEA10 | ENSG00000124260 | ASSSATGSF | 1980 | HLA-C*03:04 |
| 3392 | MAGEA10 | ENSG00000124260 | ASSSATGSF | 1980 | HLA-C*05:01 |
| 3393 | MAGEA10 | ENSG00000124260 | ASSSATGSF | 1980 | HLA-C*14:02 |
| 3394 | MAGEA10 | ENSG00000124260 | ASSSATGSF | 1980 | HLA-C*16:01 |
| 3395 | MAGEA10 | ENSG00000124260 | ASSSATGSF | 1980 | HLA-C*16:02 |
| 3396 | MAGEA10 | ENSG00000124260 | ASSSATGSF | 1980 | HLA-C*16:04 |
| 3397 | MAGEA10 | ENSG00000124260 | ASSSTSTSSSF | 1981 | HLA-A*30:02 |
| 3398 | MAGEA10 | ENSG00000124260 | ASSSTSTSSSF | 1981 | HLA-A*32:01 |
| 3399 | MAGEA10 | ENSG00000124260 | ASSSTSTSSSF | 1981 | HLA-B*58:01 |
| 3400 | MAGEA10 | ENSG00000124260 | ATTDDTTAM | 1982 | HLA-A*25:01 |
| 3401 | MAGEA10 | ENSG00000124260 | ATTDDTTAM | 1982 | HLA-A*26:01 |
| 3402 | MAGEA10 | ENSG00000124260 | ATTDDTTAM | 1982 | HLA-A*32:01 |
| 3403 | MAGEA10 | ENSG00000124260 | ATTDDTTAM | 1982 | HLA-B*15:01 |
| 3404 | MAGEA10 | ENSG00000124260 | ATTDDTTAM | 1982 | HLA-B*39:01 |
| 3405 | MAGEA10 | ENSG00000124260 | ATTDDTTAM | 1982 | HLA-B*46:01 |
| 3406 | MAGEA10 | ENSG00000124260 | ATTDDTTAM | 1982 | HLA-B*58:01 |
| 3407 | MAGEA10 | ENSG00000124260 | ATTDDTTAM | 1982 | HLA-C*01:02 |
| 3408 | MAGEA10 | ENSG00000124260 | ATTDDTTAM | 1982 | HLA-C*02:02 |
| 3409 | MAGEA10 | ENSG00000124260 | ATTDDTTAM | 1982 | HLA-C*03:03 |
| 3410 | MAGEA10 | ENSG00000124260 | ATTDDTTAM | 1982 | HLA-C*03:04 |
| 3411 | MAGEA10 | ENSG00000124260 | ATTDDTTAM | 1982 | HLA-C*04:01 |
| 3412 | MAGEA10 | ENSG00000124260 | ATTDDTTAM | 1982 | HLA-C*05:01 |
| 3413 | MAGEA10 | ENSG00000124260 | ATTDDTTAM | 1982 | HLA-C*07:06 |
| 3414 | MAGEA10 | ENSG00000124260 | ATTDDTTAM | 1982 | HLA-C*12:03 |
| 3415 | MAGEA10 | ENSG00000124260 | ATTDDTTAM | 1982 | HLA-C*14:02 |
| 3416 | MAGEA10 | ENSG00000124260 | ATTDDTTAM | 1982 | HLA-C*16:01 |
| 3417 | MAGEA10 | ENSG00000124260 | ATTDDTTAM | 1982 | HLA-C*16:02 |
| 3418 | MAGEA10 | ENSG00000124260 | ATTDDTTAM | 1982 | HLA-C*I6:04 |
| 3419 | MAGEA10 | ENSG00000124260 | DDETPNPPQSA | 1983 | HLA-B*39:01 |
| 3420 | MAGEA10 | ENSG00000124260 | DEKVTDLVQF | 1984 | HLA-B*18:01 |
| 3421 | MAGEA10 | ENSG00000124260 | DEKVTDLVQF | 1984 | HLA-B*44:02 |
| 3422 | MAGEA10 | ENSG00000124260 | DEKVTDLV | 1985 | HLA-B*18:01 |
| 3423 | MAGEA10 | ENSG00000124260 | DEKVTDLV | 1985 | HLA-B*49:01 |
| 3424 | MAGEA10 | ENSG00000124260 | DETPNPPQSA | 1986 | HLA-B*18:01 |
| 3425 | MAGEA10 | ENSG00000124260 | DETPNPPQS | 1987 | HLA-B*18:01 |
| 3426 | MAGEA10 | ENSG00000124260 | DETPNPPQ | 1988 | HLA-B*18:01 |
| 3427 | MAGEA10 | ENSG00000124260 | DGMEHLIY | 1989 | HLA-B*08:01 |
| 3428 | MAGEA10 | ENSG00000124260 | DGMEHLIY | 1989 | HLA-B*18:01 |
| 3429 | MAGEA10 | ENSG00000124260 | DGMEHLIY | 1989 | HLA-B*35:01 |
| 3430 | MAGEA10 | ENSG00000124260 | DGMEHLIY | 1989 | HLA-C*07:01 |
| 3431 | MAGEA10 | ENSG00000124260 | DGMEHLIY | 1989 | HLA-C*12:03 |
| 3432 | MAGEA10 | ENSG00000124260 | DGMLSDVQSM | 1990 | HLA-B*51:01 |
| 3433 | MAGEA10 | ENSG00000124260 | DPTGHSFVL | 1991 | HLA-B*08:01 |
| 3434 | MAGEA10 | ENSG00000124260 | DPTGHSFVL | 1991 | HLA-B*35:01 |
| 3435 | MAGEA10 | ENSG00000124260 | DPTGHSFVL | 1991 | HLA-B*35:03 |
| 3436 | MAGEA10 | ENSG00000124260 | DPTGHSFV | 1992 | HLA-B*51:01 |
| 3437 | MAGEA10 | ENSG00000124260 | DVKEVDPTGH | 1993 | HLA-A*26:01 |
| 3438 | MAGEA10 | ENSG00000124260 | DVKEVDPTGH | 1993 | HLA-A*33:01 |
| 3439 | MAGEA10 | ENSG00000124260 | DVKEVDPTGH | 1993 | HLA-A*68:01 |
| 3440 | MAGEA10 | ENSG00000124260 | EALNMMGLY | 1994 | HLA-A*01:01 |
| 3441 | MAGEA10 | ENSG00000124260 | EALNMMGLY | 1994 | HLA-A*25:01 |
| 3442 | MAGEA10 | ENSG00000124260 | EALNMMGLY | 1994 | HLA-A*26:01 |
| 3443 | MAGEA10 | ENSG00000124260 | EALNMMGLY | 1994 | HLA-A*29:02 |
| 3444 | MAGEA10 | ENSG00000124260 | EALNMMGLY | 1994 | HLA-B*35:01 |
| 3445 | MAGEA10 | ENSG00000124260 | EALNMMGL | 1995 | HLA-B*51:01 |
| 3446 | MAGEA10 | ENSG00000124260 | EASECMLLV | 1996 | HLA-A*68:02 |
| 3447 | MAGEA10 | ENSG00000124260 | EASECMLLV | 1996 | HLA-B*51:01 |
| 3448 | MAGEA10 | ENSG00000124260 | EDHFPLLF | 1997 | HLA-C*07:01 |
| 3449 | MAGEA10 | ENSG00000124260 | EESPSTLQVL | 1998 | HLA-A*25:01 |
| 3450 | MAGEA10 | ENSG00000124260 | EESPSTLQVL | 1998 | HLA-A*30:01 |
| 3451 | MAGEA10 | ENSG00000124260 | EESPSTLQVL | 1998 | HLA-B*27:02 |
| 3452 | MAGEA10 | ENSG00000124260 | EESPSTLQVL | 1998 | HLA-B*38:01 |
| 3453 | MAGEA10 | ENSG00000124260 | EESPSTLQVL | 1998 | HLA-B*39:01 |
| 3454 | MAGEA10 | ENSG00000124260 | EESPSTLQVL | 1998 | HLA-B*40:01 |
| 3455 | MAGEA10 | ENSG00000124260 | EESPSTLQVL | 1998 | HLA-B*40:02 |
| 3456 | MAGEA10 | ENSG00000124260 | EESPSTLQVL | 1998 | HLA-B*44:02 |
| 3457 | MAGEA10 | ENSG00000124260 | EESPSTLQVL | 1998 | HLA-B*44:03 |
| 3458 | MAGEA10 | ENSG00000124260 | EESPSTLQV | 1999 | HLA-A*30:01 |
| 3459 | MAGEA10 | ENSG00000124260 | EESPSTLQV | 1999 | HLA-B*13:02 |
| 3460 | MAGEA10 | ENSG00000124260 | EESPSTLQV | 1999 | HLA-B*18:01 |
| 3461 | MAGEA10 | ENSG00000124260 | EESPSTLQV | 1999 | HLA-B*37:01 |
| 3462 | MAGEA10 | ENSG00000124260 | EESPSTLQV | 1999 | HLA-B*40:01 |
| 3463 | MAGEA10 | ENSG00000124260 | EESPSTLQV | 1999 | HLA-B*44:02 |
| 3464 | MAGEA10 | ENSG00000124260 | EESPSTLQV | 1999 | HLA-B*44:03 |
| 3465 | MAGEA10 | ENSG00000124260 | EESPSTLQV | 1999 | HLA-B*49:01 |
| 3466 | MAGEA10 | ENSG00000124260 | EEVIWEAL | 2000 | HLA-B*18:01 |
| 3467 | MAGEA10 | ENSG00000124260 | EGAQAPLAV | 2001 | HLA-B*13:02 |
| 3468 | MAGEA10 | ENSG00000124260 | EGAQAPLAV | 2001 | HLA-B*51:01 |
| 3469 | MAGEA10 | ENSG00000124260 | EHLIYGEPR | 2002 | HLA-A*33:01 |
| 3470 | MAGEA10 | ENSG00000124260 | EHLIYGEPR | 2002 | HLA-A*33:03 |
| 3471 | MAGEA10 | ENSG00000124260 | EIDEKVTDL | 2003 | HLA-A*02:07 |
| 3472 | MAGEA10 | ENSG00000124260 | EIDEKVTDL | 2003 | HLA-C*05:01 |
| 3473 | MAGEA10 | ENSG00000124260 | EILESVIRNY | 2004 | HLA-A*25:01 |
| 3474 | MAGEA10 | ENSG00000124260 | EILESVIRNY | 2004 | HLA-A*26:01 |
| 3475 | MAGEA10 | ENSG00000124260 | EILESVIRNY | 2004 | HLA-A*29:02 |
| 3476 | MAGEA10 | ENSG00000124260 | EILESVIRNY | 2004 | HLA-A*30:02 |
| 3477 | MAGEA10 | ENSG00000124260 | EILESVIRNY | 2004 | HLA-A*33:01 |
| 3478 | MAGEA10 | ENSG00000124260 | EILESVIRNY | 2004 | HLA-A*33:03 |
| 3479 | MAGEA10 | ENSG00000124260 | EILESVIRNY | 2004 | HLA-B*44:02 |
| 3480 | MAGEA10 | ENSG00000124260 | EILESVIRNY | 2004 | HLA-B*44:03 |
| 3481 | MAGEA10 | ENSG00000124260 | EILESVIRNY | 2004 | HLA-C*07:06 |
| 3482 | MAGEA10 | ENSG00000124260 | EILESVIR | 2005 | HLA-A*33:03 |
| 3483 | MAGEA10 | ENSG00000124260 | EKVTDLVQF | 2006 | HLA-A*23:01 |
| 3484 | MAGEA10 | ENSG00000124260 | EPITKAEIL | 2007 | HLA-B*07:02 |
| 3485 | MAGEA10 | ENSG00000124260 | EPITKAEIL | 2007 | HLA-B*08:01 |
| 3486 | MAGEA10 | ENSG00000124260 | EPITKAEIL | 2007 | HLA-B*35:01 |
| 3487 | MAGEA10 | ENSG00000124260 | EPITKAEIL | 2007 | HLA-B*35:03 |
| 3488 | MAGEA10 | ENSG00000124260 | EPITKAEIL | 2007 | HLA-C*07:02 |
| 3489 | MAGEA10 | ENSG00000124260 | EPITKAEI | 2008 | HLA-B*08:01 |
| 3490 | MAGEA10 | ENSG00000124260 | EPITKAEI | 2008 | HLA-B*51:01 |
| 3491 | MAGEA10 | ENSG00000124260 | ESLPRSEIDEK | 2009 | HLA-A*33:01 |
| 3492 | MAGEA10 | ENSG00000124260 | ESPSTLQVL | 2010 | HLA-A*25:01 |
| 3493 | MAGEA10 | ENSG00000124260 | ESPSTLQVL | 2010 | HLA-C*01:02 |
| 3494 | MAGEA10 | ENSG00000124260 | ETPNPPQSAQI | 2011 | HLA-A*26:01 |
| 3495 | MAGEA10 | ENSG00000124260 | ETPNPPQSAQI | 2011 | HLA-A*68:02 |
| 3496 | MAGEA10 | ENSG00000124260 | ETPNPPQSA | 2012 | HLA-A*33:03 |
| 3497 | MAGEA10 | ENSG00000124260 | EVDPTGHSFVL | 2013 | HLA-A*01:01 |
| 3498 | MAGEA10 | ENSG00000124260 | EVDPTGHSFVL | 2013 | HLA-A*02:07 |
| 3499 | MAGEA10 | ENSG00000124260 | EVDPTGHSFVL | 2013 | HLA-A*68:02 |
| 3500 | MAGEA10 | ENSG00000124260 | EVDPTGHSFVL | 2013 | HLA-B*35:03 |
| 3501 | MAGEA10 | ENSG00000124260 | EVDPTGHSFVL | 2013 | HLA-B*38:01 |
| 3502 | MAGEA10 | ENSG00000124260 | EVDPTGHSFVL | 2013 | HLA-C*05:01 |
| 3503 | MAGEA10 | ENSG00000124260 | EVDPTGHSFV | 2014 | HLA-A*01:01 |
| 3504 | MAGEA10 | ENSG00000124260 | EVDPTGHSFV | 2014 | HLA-A*26:01 |
| 3505 | MAGEA10 | ENSG00000124260 | EVDPTGHSFV | 2014 | HLA-A*68:02 |
| 3506 | MAGEA10 | ENSG00000124260 | EVDPTGHSFV | 2014 | HLA-C*05:01 |
| 3507 | MAGEA10 | ENSG00000124260 | EVDPTGHSF | 2015 | HLA-A*01:01 |
| 3508 | MAGEA10 | ENSG00000124260 | EVDPTGHSF | 2015 | HLA-A*02:07 |
| 3509 | MAGEA10 | ENSG00000124260 | EVDPTGHSF | 2015 | HLA-A*23:01 |
| 3510 | MAGEA10 | ENSG00000124260 | EVDPTGHSF | 2015 | HLA-A*24:02 |
| 3511 | MAGEA10 | ENSG00000124260 | EVDPTGHSF | 2015 | HLA-A*25:01 |
| 3512 | MAGEA10 | ENSG00000124260 | EVDPTGHSF | 2015 | HLA-A*26:01 |
| 3513 | MAGEA10 | ENSG00000124260 | EVDPTGHSF | 2015 | HLA-A*29:02 |
| 3514 | MAGEA10 | ENSG00000124260 | EVDPTGHSF | 2015 | HLA-A*30:02 |
| 3515 | MAGEA10 | ENSG00000124260 | EVDPTGHSF | 2015 | HLA-A*32:01 |
| 3516 | MAGEA10 | ENSG00000124260 | EVDPTGHSF | 2015 | HLA-A*33:01 |
| 3517 | MAGEA10 | ENSG00000124260 | EVDPTGHSF | 2015 | HLA-A*33:03 |
| 3518 | MAGEA10 | ENSG00000124260 | EVDPTGHSF | 2015 | HLA-A*68:01 |
| 3519 | MAGEA10 | ENSG00000124260 | EVDPTGHSF | 2015 | HLA-B*15:01 |
| 3520 | MAGEA10 | ENSG00000124260 | EVDPTGHSF | 2015 | HLA-B*15:03 |
| 3521 | MAGEA10 | ENSG00000124260 | EVDPTGHSF | 2015 | HLA-B*18:01 |
| 3522 | MAGEA10 | ENSG00000124260 | EVDPTGHSF | 2015 | HLA-B*27:05 |
| 3523 | MAGEA10 | ENSG00000124260 | EVDPTGHSF | 2015 | HLA-B*35:01 |
| 3524 | MAGEA10 | ENSG00000124260 | EVDPTGHSF | 2015 | HLA-B*35:03 |
| 3525 | MAGEA10 | ENSG00000124260 | EVDPTGHSF | 2015 | HLA-B*38:01 |
| 3526 | MAGEA10 | ENSG00000124260 | EVDPTGHSF | 2015 | HLA-B*39:01 |
| 3527 | MAGEA10 | ENSG00000124260 | EVDPTGHSF | 2015 | HLA-B*44:02 |
| 3528 | MAGEA10 | ENSG00000124260 | EVDPTGHSF | 2015 | HLA-B*44:03 |
| 3529 | MAGEA10 | ENSG00000124260 | EVDPTGHSF | 2015 | HLA-B*46:01 |
| 3530 | MAGEA10 | ENSG00000124260 | EVDPTGHSF | 2015 | HLA-B*55:01 |
| 3531 | MAGEA10 | ENSG00000124260 | EVDPTGHSF | 2015 | HLA-B*57:01 |
| 3532 | MAGEA10 | ENSG00000124260 | EVDPTGHSF | 2015 | HLA-B*58:01 |
| 3533 | MAGEA10 | ENSG00000124260 | EVDPTGHSF | 2015 | HLA-C*02:02 |
| 3534 | MAGEA10 | ENSG00000124260 | EVDPTGHSF | 2015 | HLA-C*03:03 |
| 3535 | MAGEA10 | ENSG00000124260 | EVDPTGHSF | 2015 | HLA-C*03:04 |
| 3536 | MAGEA10 | ENSG00000124260 | EVDPTGHSF | 2015 | HLA-C*04:01 |
| 3537 | MAGEA10 | ENSG00000124260 | EVDPTGHSF | 2015 | HLA-C*05:01 |
| 3538 | MAGEA10 | ENSG00000124260 | EVDPTGHSF | 2015 | HLA-C*07:04 |
| 3539 | MAGEA10 | ENSG00000124260 | EVDPTGHSF | 2015 | HLA-C*07:06 |
| 3540 | MAGEA10 | ENSG00000124260 | EVDPTGHSF | 2015 | HLA-C*14:02 |
| 3541 | MAGEA10 | ENSG00000124260 | EVDPTGHSF | 2015 | HLA-C*16:02 |
| 3542 | MAGEA10 | ENSG00000124260 | EVDPTGHSF | 2015 | HLA-C*16:04 |
| 3543 | MAGEA10 | ENSG00000124260 | EVIWEALNMM | 2016 | HLA-A*25:01 |
| 3544 | MAGEA10 | ENSG00000124260 | EVIWEALNMM | 2016 | HLA-A*26:01 |
| 3545 | MAGEA10 | ENSG00000124260 | EVIWEALNM | 2017 | HLA-A*25:01 |
| 3546 | MAGEA10 | ENSG00000124260 | EVIWEALNM | 2017 | HLA-A*26:01 |
| 3547 | MAGEA10 | ENSG00000124260 | FGIDVKEV | 2018 | HLA-B*51:01 |
| 3548 | MAGEA10 | ENSG00000124260 | FGIDVKEV | 2018 | HLA-C*12:03 |
| 3549 | MAGEA10 | ENSG00000124260 | FGIDVKEV | 2018 | HLA-C*16:02 |
| 3550 | MAGEA10 | ENSG00000124260 | FLWGPRAHAEI | 2019 | HLA-A*02:01 |
| 3551 | MAGEA10 | ENSG00000124260 | FLWGPRAHAEI | 2019 | HLA-A*02:07 |
| 3552 | MAGEA10 | ENSG00000124260 | FPLLFSEASEC | 2020 | HLA-B*54:01 |
| 3553 | MAGEA10 | ENSG00000124260 | FPLLFSEAS | 2021 | HLA-B*54:01 |
| 3554 | MAGEA10 | ENSG00000124260 | FPLLFSEA | 2022 | HLA-B*51:01 |
| 3555 | MAGEA10 | ENSG00000124260 | FPLLFSEA | 2022 | HLA-B*54:01 |
| 3556 | MAGEA10 | ENSG00000124260 | FPLLFSEA | 2022 | HLA-B*55:01 |
| 3557 | MAGEA10 | ENSG00000124260 | FPLLFSEA | 2022 | HLA-B*56:01 |
| 3558 | MAGEA10 | ENSG00000124260 | FPLWYEEAL | 2023 | HLA-B*35:01 |
| 3559 | MAGEA10 | ENSG00000124260 | FPLWYEEAL | 2023 | HLA-B*35:03 |
| 3560 | MAGEA10 | ENSG00000124260 | FPLWYEEAL | 2023 | HLA-B*54:01 |
| 3561 | MAGEA10 | ENSG00000124260 | FPLWYEEA | 2024 | HLA-B*54:01 |
| 3562 | MAGEA10 | ENSG00000124260 | FPSSFPSSSS | 2025 | HLA-B*54:01 |
| 3563 | MAGEA10 | ENSG00000124260 | FPSSFPSSSS | 2025 | HLA-B*56:01 |
| 3564 | MAGEA10 | ENSG00000124260 | FPSSFPSSS | 2026 | HLA-B*54:01 |
| 3565 | MAGEA10 | ENSG00000124260 | FPSSFPSSS | 2026 | HLA-B*56:01 |
| 3566 | MAGEA10 | ENSG00000124260 | FPSSFPSS | 2027 | HLA-B*54:01 |
| 3567 | MAGEA10 | ENSG00000124260 | FPSSSSSSS | 2028 | HLA-B*39:01 |
| 3568 | MAGEA10 | ENSG00000124260 | FSEASECML | 2029 | HLA-C*03:03 |
| 3569 | MAGEA10 | ENSG00000124260 | FSEASECML | 2029 | HLA-C*03:04 |
| 3570 | MAGEA10 | ENSG00000124260 | FSEASECML | 2029 | HLA-C*05:01 |
| 3571 | MAGEA10 | ENSG00000124260 | FVLVTSLGLTY | 2030 | HLA-A*29:02 |
| 3572 | MAGEA10 | ENSG00000124260 | GHSFVLVTSL | 2031 | HLA-B*38:01 |
| 3573 | MAGEA10 | ENSG00000124260 | GLYDGMEHLIY | 2032 | HLA-A*01:01 |
| 3574 | MAGEA10 | ENSG00000124260 | GLYDGMEHLIY | 2032 | HLA-A*03:01 |
| 3575 | MAGEA10 | ENSG00000124260 | GLYDGMEHLIY | 2032 | HLA-A*29:02 |
| 3576 | MAGEA10 | ENSG00000124260 | GLYDGMEHLI | 2033 | HLA-A*02:01 |
| 3577 | MAGEA10 | ENSG00000124260 | GLYDGMEHLI | 2033 | HLA-A*02:03 |
| 3578 | MAGEA10 | ENSG00000124260 | GLYDGMEHLI | 2033 | HLA-A*02:04 |
| 3579 | MAGEA10 | ENSG00000124260 | GLYDGMEHLI | 2033 | HLA-A*02:07 |
| 3580 | MAGEA10 | ENSG00000124260 | GLYDGMEHLI | 2033 | HLA-A*03:01 |
| 3581 | MAGEA10 | ENSG00000124260 | GLYDGMEHLI | 2033 | HLA-A*30:01 |
| 3582 | MAGEA10 | ENSG00000124260 | GLYDGMEHLI | 2033 | HLA-B*13:02 |
| 3583 | MAGEA10 | ENSG00000124260 | GLYDGMEHLI | 2033 | HLA-B*55:01 |
| 3584 | MAGEA10 | ENSG00000124260 | GLYDGMEHLI | 2033 | HLA-C*06:02 |
| 3585 | MAGEA10 | ENSG00000124260 | GLYDGMEHL | 2034 | HLA-A*02:01 |
| 3586 | MAGEA10 | ENSG00000124260 | GLYDGMEHL | 2034 | HLA-A*02:03 |
| 3587 | MAGEA10 | ENSG00000124260 | GLYDGMEHL | 2034 | HLA-A*02:04 |
| 3588 | MAGEA10 | ENSG00000124260 | GLYDGMEHL | 2034 | HLA-A*02:07 |
| 3589 | MAGEA10 | ENSG00000124260 | GLYDGMEHL | 2034 | HLA-A*03:01 |
| 3590 | MAGEA10 | ENSG00000124260 | GLYDGMEHL | 2034 | HLA-A*30:01 |
| 3591 | MAGEA10 | ENSG00000124260 | GLYDGMEHL | 2034 | HLA-A*68:02 |
| 3592 | MAGEA10 | ENSG00000124260 | GLYDGMEHL | 2034 | HLA-B*13:02 |
| 3593 | MAGEA10 | ENSG00000124260 | GLYDGMEHL | 2034 | HLA-B*15:01 |
| 3594 | MAGEA10 | ENSG00000124260 | GLYDGMEHL | 2034 | HLA-B*15:03 |
| 3595 | MAGEA10 | ENSG00000124260 | GLYDGMEHL | 2034 | HLA-B*40:01 |
| 3596 | MAGEA10 | ENSG00000124260 | GLYDGMEHL | 2034 | HLA-B*46:01 |
| 3597 | MAGEA10 | ENSG00000124260 | GLYDGMEHL | 2034 | HLA-B*55:01 |
| 3598 | MAGEA10 | ENSG00000124260 | GLYDGMEHL | 2034 | HLA-C*02:02 |
| 3599 | MAGEA10 | ENSG00000124260 | GMLSDVQSMPK | 2035 | HLA-A*03:01 |
| 3600 | MAGEA10 | ENSG00000124260 | GMLSDVQSMPK | 2035 | HLA-A*03:02 |
| 3601 | MAGEA10 | ENSG00000124260 | GMLSDVQSMPK | 2035 | HLA-A*11:01 |
| 3602 | MAGEA10 | ENSG00000124260 | GMLSDVQSMPK | 2035 | HLA-B*27:05 |
| 3603 | MAGEA10 | ENSG00000124260 | GMLSDVQSM | 2036 | HLA-A*02:01 |
| 3604 | MAGEA10 | ENSG00000124260 | GMLSDVQSM | 2036 | HLA-A*02:04 |
| 3605 | MAGEA10 | ENSG00000124260 | GMLSDVQSM | 2036 | HLA-A*23:01 |
| 3606 | MAGEA10 | ENSG00000124260 | GMLSDVQSM | 2036 | HLA-A*32:01 |
| 3607 | MAGEA10 | ENSG00000124260 | GMLSDVQSM | 2036 | HLA-B*13:02 |
| 3608 | MAGEA10 | ENSG00000124260 | GMLSDVQSM | 2036 | HLA-B*15:01 |
| 3609 | MAGEA10 | ENSG00000124260 | GMLSDVQSM | 2036 | HLA-B*15:03 |
| 3610 | MAGEA10 | ENSG00000124260 | GMLSDVQSM | 2036 | HLA-B*27:05 |
| 3611 | MAGEA10 | ENSG00000124260 | GMLSDVQSM | 2036 | HLA-B*37:01 |
| 3612 | MAGEA10 | ENSG00000124260 | GMLSDVQSM | 2036 | HLA-B*46:01 |
| 3613 | MAGEA10 | ENSG00000124260 | GMLSDVQSM | 2036 | HLA-B*55:01 |
| 3614 | MAGEA10 | ENSG00000124260 | GMLSDVQSM | 2036 | HLA-B*58:01 |
| 3615 | MAGEA10 | ENSG00000124260 | GMLSDVQSM | 2036 | HLA-C*01:02 |
| 3616 | MAGEA10 | ENSG00000124260 | GMLSDVQSM | 2036 | HLA-C*07:04 |
| 3617 | MAGEA10 | ENSG00000124260 | GMLSDVQSM | 2036 | HLA-C*14:02 |
| 3618 | MAGEA10 | ENSG00000124260 | GPRAHAEI | 2037 | HLA-B*07:02 |
| 3619 | MAGEA10 | ENSG00000124260 | GPRAHAEI | 2037 | HLA-C*07:02 |
| 3620 | MAGEA10 | ENSG00000124260 | GSDPARYEFLW | 2038 | HLA-B*57:01 |
| 3621 | MAGEA10 | ENSG00000124260 | GSDPARYEF | 2039 | HLA-A*01:01 |
| 3622 | MAGEA10 | ENSG00000124260 | GSDPARYEF | 2039 | HLA-A*30:02 |
| 3623 | MAGEA10 | ENSG00000124260 | GSDPARYEF | 2039 | HLA-A*32:01 |
| 3624 | MAGEA10 | ENSG00000124260 | GSDPARYEF | 2039 | HLA-B*15:01 |
| 3625 | MAGEA10 | ENSG00000124260 | GSDPARYEF | 2039 | HLA-B*38:01 |
| 3626 | MAGEA10 | ENSG00000124260 | GSDPARYEF | 2039 | HLA-B*57:01 |
| 3627 | MAGEA10 | ENSG00000124260 | GSDPARYEF | 2039 | HLA-B*58:01 |
| 3628 | MAGEA10 | ENSG00000124260 | GSDPARYEF | 2039 | HLA-C*02:02 |
| 3629 | MAGEA10 | ENSG00000124260 | GSDPARYEF | 2039 | HLA-C*03:04 |
| 3630 | MAGEA10 | ENSG00000124260 | GSDPARYEF | 2039 | HLA-C*05:01 |
| 3631 | MAGEA10 | ENSG00000124260 | GSDPARYEF | 2039 | HLA-C*16:01 |
| 3632 | MAGEA10 | ENSG00000124260 | GSDPARYEF | 2039 | HLA-C*16:04 |
| 3633 | MAGEA10 | ENSG00000124260 | GSDPRSFPLWY | 2040 | HLA-A*01:01 |
| 3634 | MAGEA10 | ENSG00000124260 | GSDPRSFPLW | 2041 | HLA-B*57:01 |
| 3635 | MAGEA10 | ENSG00000124260 | GSDPRSFPL | 2042 | HLA-A*01:01 |
| 3636 | MAGEA10 | ENSG00000124260 | GSDPRSFPL | 2042 | HLA-C*03:04 |
| 3637 | MAGEA10 | ENSG00000124260 | GSDPRSFPL | 2042 | HLA-C*05:01 |
| 3638 | MAGEA10 | ENSG00000124260 | HAEIRKMSL | 2043 | HLA-B*08:01 |
| 3639 | MAGEA10 | ENSG00000124260 | HSFVLVTSL | 2044 | HLA-A*68:02 |
| 3640 | MAGEA10 | ENSG00000124260 | HSFVLVTSL | 2044 | HLA-B*08:01 |
| 3641 | MAGEA10 | ENSG00000124260 | HSFVLVTSL | 2044 | HLA-B*15:03 |
| 3642 | MAGEA10 | ENSG00000124260 | HSFVLVTSL | 2044 | HLA-B*18:01 |
| 3643 | MAGEA10 | ENSG00000124260 | HSFVLVTSL | 2044 | HLA-B*35:01 |
| 3644 | MAGEA10 | ENSG00000124260 | HSFVLVTSL | 2044 | HLA-B*40:01 |
| 3645 | MAGEA10 | ENSG00000124260 | HSFVLVTSL | 2044 | HLA-B*40:02 |
| 3646 | MAGEA10 | ENSG00000124260 | HSFVLVTSL | 2044 | HLA-B*46:01 |
| 3647 | MAGEA10 | ENSG00000124260 | HSFVLVTSL | 2044 | HLA-B*58:01 |
| 3648 | MAGEA10 | ENSG00000124260 | HSFVLVTSL | 2044 | HLA-C*02:02 |
| 3649 | MAGEA10 | ENSG00000124260 | HSFVLVTSL | 2044 | HLA-C*03:03 |
| 3650 | MAGEA10 | ENSG00000124260 | HSFVLVTSL | 2044 | HLA-C*03:04 |
| 3651 | MAGEA10 | ENSG00000124260 | HSFVLVTSL | 2044 | HLA-C*12:03 |
| 3652 | MAGEA10 | ENSG00000124260 | IACSSPSVV | 2045 | HLA-B*51:01 |
| 3653 | MAGEA10 | ENSG00000124260 | IACSSPSVV | 2045 | HLA-C*12:03 |
| 3654 | MAGEA10 | ENSG00000124260 | IACSSPSV | 2046 | HLA-B*51:01 |
| 3655 | MAGEA10 | ENSG00000124260 | IATTDDTTAM | 2047 | HLA-B*35:01 |
| 3656 | MAGEA10 | ENSG00000124260 | IATTDDTTAM | 2047 | HLA-B*35:03 |
| 3657 | MAGEA10 | ENSG00000124260 | IATTDDTTAM | 2047 | HLA-B*39:01 |
| 3658 | MAGEA10 | ENSG00000124260 | IATTDDTTAM | 2047 | HLA-B*55:01 |
| 3659 | MAGEA10 | ENSG00000124260 | IATTDDTTAM | 2047 | HLA-C*03:03 |
| 3660 | MAGEA10 | ENSG00000124260 | IATTDDTTAM | 2047 | HLA-C*03:04 |
| 3661 | MAGEA10 | ENSG00000124260 | IATTDDTTAM | 2047 | HLA-C*05:01 |
| 3662 | MAGEA10 | ENSG00000124260 | IATTDDTTA | 2048 | HLA-B*35:03 |
| 3663 | MAGEA10 | ENSG00000124260 | IDEKVTDL | 2049 | HLA-B*37:01 |
| 3664 | MAGEA10 | ENSG00000124260 | ILESVIRNY | 2050 | HLA-A*01:01 |
| 3665 | MAGEA10 | ENSG00000124260 | ILESVIRNY | 2050 | HLA-A*02:07 |
| 3666 | MAGEA10 | ENSG00000124260 | ILESVIRNY | 2050 | HLA-A*03:01 |
| 3667 | MAGEA10 | ENSG00000124260 | ILESVIRNY | 2050 | HLA-A*03:02 |
| 3668 | MAGEA10 | ENSG00000124260 | ILESVIRNY | 2050 | HLA-A*29:02 |
| 3669 | MAGEA10 | ENSG00000124260 | ILESVIRNY | 2050 | HLA-A*30:02 |
| 3670 | MAGEA10 | ENSG00000124260 | ILESVIRNY | 2050 | HLA-A*32:01 |
| 3671 | MAGEA10 | ENSG00000124260 | ILESVIRNY | 2050 | HLA-B*15:01 |
| 3672 | MAGEA10 | ENSG00000124260 | ILESVIRNY | 2050 | HLA-B*15:03 |
| 3673 | MAGEA10 | ENSG00000124260 | ILESVIRNY | 2050 | HLA-B*44:02 |
| 3674 | MAGEA10 | ENSG00000124260 | ILESVIRNY | 2050 | HLA-B*44:03 |
| 3675 | MAGEA10 | ENSG00000124260 | ILESVIRNY | 2050 | HLA-B*46:01 |
| 3676 | MAGEA10 | ENSG00000124260 | ILESVIRNY | 2050 | HLA-B*57:01 |
| 3677 | MAGEA10 | ENSG00000124260 | ILESVIRNY | 2050 | HLA-B*58:01 |
| 3678 | MAGEA10 | ENSG00000124260 | ILESVIRNY | 2050 | HLA-C*02:02 |
| 3679 | MAGEA10 | ENSG00000124260 | ILESVIRNY | 2050 | HLA-C*07:04 |
| 3680 | MAGEA10 | ENSG00000124260 | ILESVIRNY | 2050 | HLA-C*16:01 |
| 3681 | MAGEA10 | ENSG00000124260 | ILESVIRNY | 2050 | HLA-C*16:02 |
| 3682 | MAGEA10 | ENSG00000124260 | ILILSIVFI | 2051 | HLA-A*02:01 |
| 3683 | MAGEA10 | ENSG00000124260 | ILILSIVFI | 2051 | HLA-A*02:04 |
| 3684 | MAGEA10 | ENSG00000124260 | ILSIVFIEGY | 2052 | HLA-A*01:01 |
| 3685 | MAGEA10 | ENSG00000124260 | IPSTPEEVSA | 2053 | HLA-B*35:03 |
| 3686 | MAGEA10 | ENSG00000124260 | IPSTPEEVSA | 2053 | HLA-B*54:01 |
| 3687 | MAGEA10 | ENSG00000124260 | IPSTPEEVSA | 2053 | HLA-B*55:01 |
| 3688 | MAGEA10 | ENSG00000124260 | IPSTPEEVSA | 2053 | HLA-B*56:01 |
| 3689 | MAGEA10 | ENSG00000124260 | IYGEPRKLL | 2054 | HLA-A*24:02 |
| 3690 | MAGEA10 | ENSG00000124260 | IYGEPRKL | 2055 | HLA-A*23:01 |
| 3691 | MAGEA10 | ENSG00000124260 | IYGEPRKL | 2055 | HLA-A*24:02 |
| 3692 | MAGEA10 | ENSG00000124260 | IYGEPRKL | 2055 | HLA-C*06:02 |
| 3693 | MAGEA10 | ENSG00000124260 | IYGEPRKL | 2055 | HLA-C*16:01 |
| 3694 | MAGEA10 | ENSG00000124260 | KEESPSTLQVL | 2056 | HLA-A*30:01 |
| 3695 | MAGEA10 | ENSG00000124260 | KEESPSTLQVL | 2056 | HLA-B*40:01 |
| 3696 | MAGEA10 | ENSG00000124260 | KEESPSTLQVL | 2056 | HLA-B*40:02 |
| 3697 | MAGEA10 | ENSG00000124260 | KEESPSTLQVL | 2056 | HLA-B*44:03 |
| 3698 | MAGEA10 | ENSG00000124260 | KEESPSTLQVL | 2056 | HLA-B*49:01 |
| 3699 | MAGEA10 | ENSG00000124260 | KEESPSTLQV | 2057 | HLA-B*40:01 |
| 3700 | MAGEA10 | ENSG00000124260 | KEESPSTLQV | 2057 | HLA-B*49:01 |
| 3701 | MAGEA10 | ENSG00000124260 | KEESPSTL | 2058 | HLA-B*40:01 |
| 3702 | MAGEA10 | ENSG00000124260 | KEPITKAEIL | 2059 | HLA-B*40:01 |
| 3703 | MAGEA10 | ENSG00000124260 | KEPITKAEI | 2060 | HLA-B*37:01 |
| 3704 | MAGEA10 | ENSG00000124260 | KEPITKAEI | 2060 | HLA-B*40:01 |
| 3705 | MAGEA10 | ENSG00000124260 | KEPITKAEI | 2060 | HLA-B*49:01 |
| 3706 | MAGEA10 | ENSG00000124260 | KEVDPTGHSFV | 2061 | HLA-B*49:01 |
| 3707 | MAGEA10 | ENSG00000124260 | KEVDPTGHSF | 379 | HLA-A*23:01 |
| 3708 | MAGEA10 | ENSG00000124260 | KEVDPTGHSF | 379 | HLA-A*24:02 |
| 3709 | MAGEA10 | ENSG00000124260 | KEVDPTGHSF | 379 | HLA-A*25:01 |
| 3710 | MAGEA10 | ENSG00000124260 | KEVDPTGHSF | 379 | HLA-A*30:01 |
| 3711 | MAGEA10 | ENSG00000124260 | KEVDPTGHSF | 379 | HLA-A*30:02 |
| 3712 | MAGEA10 | ENSG00000124260 | KEVDPTGHSF | 379 | HLA-A*32:01 |
| 3713 | MAGEA10 | ENSG00000124260 | KEVDPTGHSF | 379 | HLA-B*15:01 |
| 3714 | MAGEA10 | ENSG00000124260 | KEVDPTGHSF | 379 | HLA-B*15:03 |
| 3715 | MAGEA10 | ENSG00000124260 | KEVDPTGHSF | 379 | HLA-B*18:01 |
| 3716 | MAGEA10 | ENSG00000124260 | KEVDPTGHSF | 379 | HLA-B*27:02 |
| 3717 | MAGEA10 | ENSG00000124260 | KEVDPTGHSF | 379 | HLA-B*27:05 |
| 3718 | MAGEA10 | ENSG00000124260 | KEVDPTGHSF | 379 | HLA-B*37:01 |
| 3719 | MAGEA10 | ENSG00000124260 | KEVDPTGHSF | 379 | HLA-B*38:01 |
| 3720 | MAGEA10 | ENSG00000124260 | KEVDPTGHSF | 379 | HLA-B*40:01 |
| 3721 | MAGEA10 | ENSG00000124260 | KEVDPTGHSF | 379 | HLA-B*40:02 |
| 3722 | MAGEA10 | ENSG00000124260 | KEVDPTGHSF | 379 | HLA-B*44:02 |
| 3723 | MAGEA10 | ENSG00000124260 | KEVDPTGHSF | 379 | HLA-B*44:03 |
| 3724 | MAGEA10 | ENSG00000124260 | KEVDPTGHSF | 379 | HLA-B*49:01 |
| 3725 | MAGEA10 | ENSG00000124260 | KEVDPTGHSF | 379 | HLA-B*57:01 |
| 3726 | MAGEA10 | ENSG00000124260 | KEVDPTGHSF | 379 | HLA-B*58:01 |
| 3727 | MAGEA10 | ENSG00000124260 | KEVDPTGHSF | 379 | HLA-C*02:02 |
| 3728 | MAGEA10 | ENSG00000124260 | KEVDPTGHSF | 379 | HLA-C*03:04 |
| 3729 | MAGEA10 | ENSG00000124260 | KEVDPTGHSF | 379 | HLA-C*16:01 |
| 3730 | MAGEA10 | ENSG00000124260 | KEVDPTGHSF | 379 | HLA-C*16:04 |
| 3731 | MAGEA10 | ENSG00000124260 | KVNGSDPRSF | 2062 | HLA-A*03:02 |
| 3732 | MAGEA10 | ENSG00000124260 | KVNGSDPRSF | 2062 | HLA-A*25:01 |
| 3733 | MAGEA10 | ENSG00000124260 | KVNGSDPRSF | 2062 | HLA-A*30:02 |
| 3734 | MAGEA10 | ENSG00000124260 | KVNGSDPRSF | 2062 | HLA-A*32:01 |
| 3735 | MAGEA10 | ENSG00000124260 | KVNGSDPRSF | 2062 | HLA-B*57:01 |
| 3736 | MAGEA10 | ENSG00000124260 | KVNGSDPRSF | 2062 | HLA-B*58:01 |
| 3737 | MAGEA10 | ENSG00000124260 | KVTDLVQFLL | 2063 | HLA-A*02:04 |
| 3738 | MAGEA10 | ENSG00000124260 | KVTDLVQFL | 2064 | HLA-A*02:01 |
| 3739 | MAGEA10 | ENSG00000124260 | KVTDLVQFL | 2064 | HLA-A*02:03 |
| 3740 | MAGEA10 | ENSG00000124260 | KVTDLVQFL | 2064 | HLA-A*02:04 |
| 3741 | MAGEA10 | ENSG00000124260 | KVTDLVQFL | 2064 | HLA-A*02:07 |
| 3742 | MAGEA10 | ENSG00000124260 | KVTDLVQFL | 2064 | HLA-A*24:02 |
| 3743 | MAGEA10 | ENSG00000124260 | KVTDLVQFL | 2064 | HLA-A*31:01 |
| 3744 | MAGEA10 | ENSG00000124260 | KVTDLVQFL | 2064 | HLA-A*32:01 |
| 3745 | MAGEA10 | ENSG00000124260 | KVTDLVQFL | 2064 | HLA-A*68:02 |
| 3746 | MAGEA10 | ENSG00000124260 | KVTDLVQFL | 2064 | HLA-B*13:02 |
| 3747 | MAGEA10 | ENSG00000124260 | KVTDLVQFL | 2064 | HLA-B*27:05 |
| 3748 | MAGEA10 | ENSG00000124260 | KVTDLVQFL | 2064 | HLA-B*58:01 |
| 3749 | MAGEA10 | ENSG00000124260 | KVTDLVQF | 2065 | HLA-A*32:01 |
| 3750 | MAGEA10 | ENSG00000124260 | LEGAQAPLAV | 2066 | HLA-B*49:01 |
| 3751 | MAGEA10 | ENSG00000124260 | LEGAQAPLA | 2067 | HLA-B*49:01 |
| 3752 | MAGEA10 | ENSG00000124260 | LEGAQAPL | 2068 | HLA-B*18:01 |
| 3753 | MAGEA10 | ENSG00000124260 | LESVIRNY | 2069 | HLA-B*I8:01 |
| 3754 | MAGEA10 | ENSG00000124260 | LESVIRNY | 2069 | HLA-B*37:01 |
| 3755 | MAGEA10 | ENSG00000124260 | LESVIRNY | 2069 | HLA-B*44:02 |
| 3756 | MAGEA10 | ENSG00000124260 | LESVIRNY | 2069 | HLA-B*44:03 |
| 3757 | MAGEA10 | ENSG00000124260 | LESVIRNY | 2069 | HLA-C*02:02 |
| 3758 | MAGEA10 | ENSG00000124260 | LESVIRNY | 2069 | HLA-C*16:01 |
| 3759 | MAGEA10 | ENSG00000124260 | LESVIRNY | 2069 | HLA-C*16:04 |
| 3760 | MAGEA10 | ENSG00000124260 | LFSEASECM | 2070 | HLA-C*14:02 |
| 3761 | MAGEA10 | ENSG00000124260 | LIPSTPEEV | 2071 | HLA-A*02:01 |
| 3762 | MAGEA10 | ENSG00000124260 | LIPSTPEEV | 2071 | HLA-A*02:07 |
| 3763 | MAGEA10 | ENSG00000124260 | LIYGEPRKL | 2072 | HLA-A*03:01 |
| 3764 | MAGEA10 | ENSG00000124260 | LLFSEASECML | 2073 | HLA-A*02:01 |
| 3765 | MAGEA10 | ENSG00000124260 | LLFSEASEC | 2074 | HLA-A*02:01 |
| 3766 | MAGEA10 | ENSG00000124260 | LQSQSETQGL | 2075 | HLA-B*27:05 |
| 3767 | MAGEA10 | ENSG00000124260 | LQSQSETQGL | 2075 | HLA-B*38:01 |
| 3768 | MAGEA10 | ENSG00000124260 | LQSQSETQGL | 2075 | HLA-B*39:01 |
| 3769 | MAGEA10 | ENSG00000124260 | LQVLPDSESL | 2076 | HLA-B*39:01 |
| 3770 | MAGEA10 | ENSG00000124260 | LSDVQSMPK | 2077 | HLA-A*01:01 |
| 3771 | MAGEA10 | ENSG00000124260 | LSDVQSMPK | 2077 | HLA-A*03:01 |
| 3772 | MAGEA10 | ENSG00000124260 | LSDVQSMPK | 2077 | HLA-A*03:02 |
| 3773 | MAGEA10 | ENSG00000124260 | LSDVQSMPK | 2077 | HLA-A*11:01 |
| 3774 | MAGEA10 | ENSG00000124260 | LSDVQSMPK | 2077 | HLA-B*27:02 |
| 3775 | MAGEA10 | ENSG00000124260 | LSIVFIEGY | 2078 | HLA-A*01:01 |
| 3776 | MAGEA10 | ENSG00000124260 | LSIVFIEGY | 2078 | HLA-A*30:02 |
| 3777 | MAGEA10 | ENSG00000124260 | LSIVFIEGY | 2078 | HLA-B*46:01 |
| 3778 | MAGEA10 | ENSG00000124260 | LSIVFIEGY | 2078 | HLA-B*57:01 |
| 3779 | MAGEA10 | ENSG00000124260 | LTQDWVQENYL | 2079 | HLA-C*04:01 |
| 3780 | MAGEA10 | ENSG00000124260 | LTQDWVQENY | 2080 | HLA-A*01:01 |
| 3781 | MAGEA10 | ENSG00000124260 | LTQDWVQENY | 2080 | HLA-A*29:02 |
| 3782 | MAGEA10 | ENSG00000124260 | LTQDWVQENY | 2080 | HLA-A*30:02 |
| 3783 | MAGEA10 | ENSG00000124260 | LTQDWVQENY | 2080 | HLA-A*32:01 |
| 3784 | MAGEA10 | ENSG00000124260 | LTQDWVQENY | 2080 | HLA-B*57:01 |
| 3785 | MAGEA10 | ENSG00000124260 | LTQDWVQENY | 2080 | HLA-B*58:01 |
| 3786 | MAGEA10 | ENSG00000124260 | LTYDGMLSDV | 2081 | HLA-A*02:03 |
| 3787 | MAGEA10 | ENSG00000124260 | LTYDGMLSDV | 2081 | HLA-A*68:02 |
| 3788 | MAGEA10 | ENSG00000124260 | LVFGEDVKEV | 2082 | HLA-A*02:03 |
| 3789 | MAGEA10 | ENSG00000124260 | LVFGIDVKEV | 2082 | HLA-A*68:02 |
| 3790 | MAGEA10 | ENSG00000124260 | LVFGIDVK | 2083 | HLA-B*27:02 |
| 3791 | MAGEA10 | ENSG00000124260 | LVQFLLFKY | 2084 | HLA-A*29:02 |
| 3792 | MAGEA10 | ENSG00000124260 | LVTSLGLTY | 2085 | HLA-A*01:01 |
| 3793 | MAGEA10 | ENSG00000124260 | LVTSLGLTY | 2085 | HLA-A*03:01 |
| 3794 | MAGEA10 | ENSG00000124260 | LVTSLGLTY | 2085 | HLA-A*25:01 |
| 3795 | MAGEA10 | ENSG00000124260 | LVTSLGLTY | 2085 | HLA-A*26:01 |
| 3796 | MAGEA10 | ENSG00000124260 | LVTSLGLTY | 2085 | HLA-A*29:02 |
| 3797 | MAGEA10 | ENSG00000124260 | LVTSLGLTY | 2085 | HLA-A*30:02 |
| 3798 | MAGEA10 | ENSG00000124260 | LVTSLGLTY | 2085 | HLA-A*32:01 |
| 3799 | MAGEA10 | ENSG00000124260 | LVTSLGLTY | 2085 | HLA-B*15:01 |
| 3800 | MAGEA10 | ENSG00000124260 | LVTSLGLTY | 2085 | HLA-B*15:03 |
| 3801 | MAGEA10 | ENSG00000124260 | LVTSLGLTY | 2085 | HLA-B*18:01 |
| 3802 | MAGEA10 | ENSG00000124260 | LVTSLGLTY | 2085 | HLA-B*35:01 |
| 3803 | MAGEA10 | ENSG00000124260 | LVTSLGLTY | 2085 | HLA-B*46:01 |
| 3804 | MAGEA10 | ENSG00000124260 | LVTSLGLTY | 2085 | HLA-B*58:01 |
| 3805 | MAGEA10 | ENSG00000124260 | LVTSLGLTY | 2085 | HLA-C*02:02 |
| 3806 | MAGEA10 | ENSG00000124260 | LVTSLGLTY | 2085 | HLA-C*07:04 |
| 3807 | MAGEA10 | ENSG00000124260 | LVTSLGLTY | 2085 | HLA-C*14:02 |
| 3808 | MAGEA10 | ENSG00000124260 | LVTSLGLTY | 2085 | HLA-C*16:01 |
| 3809 | MAGEA10 | ENSG00000124260 | LVTSLGLTY | 2085 | HLA-C*16:02 |
| 3810 | MAGEA10 | ENSG00000124260 | LVTSLGLTY | 2085 | HLA-C*16:04 |
| 3811 | MAGEA10 | ENSG00000124260 | LYDGMEHLIY | 2086 | HLA-A*29:02 |
| 3812 | MAGEA10 | ENSG00000124260 | LYDGMEHLIY | 2086 | HLA-C*07:01 |
| 3813 | MAGEA10 | ENSG00000124260 | LYDGMEHLI | 2087 | HLA-A*02:07 |
| 3814 | MAGEA10 | ENSG00000124260 | LYDGMEHLI | 2087 | HLA-A*23:01 |
| 3815 | MAGEA10 | ENSG00000124260 | LYDGMEHLI | 2087 | HLA-A*24:02 |
| 3816 | MAGEA10 | ENSG00000124260 | LYDGMEHLI | 2087 | HLA-B*35:01 |
| 3817 | MAGEA10 | ENSG00000124260 | LYDGMEHLI | 2087 | HLA-B*35:03 |
| 3818 | MAGEA10 | ENSG00000124260 | LYDGMEHLI | 2087 | HLA-B*38:01 |
| 3819 | MAGEA10 | ENSG00000124260 | LYDGMEHLI | 2087 | HLA-C*04:01 |
| 3820 | MAGEA10 | ENSG00000124260 | LYDGMEHL | 2088 | HLA-C*04:01 |
| 3821 | MAGEA10 | ENSG00000124260 | MASASSSAT | 2089 | HLA-C*03:04 |
| 3822 | MAGEA10 | ENSG00000124260 | MASASSSA | 2090 | HLA-B*54:01 |
| 3823 | MAGEA10 | ENSG00000124260 | MEHLIYGEP | 2091 | HLA-B*40:02 |
| 3824 | MAGEA10 | ENSG00000124260 | MLLVFGIDV | 2092 | HLA-A*02:01 |
| 3825 | MAGEA10 | ENSG00000124260 | MLLVFGIDV | 2092 | HLA-A*02:04 |
| 3826 | MAGEA10 | ENSG00000124260 | MLSDVQSMPKT | 2093 | HLA-C*06:02 |
| 3827 | MAGEA10 | ENSG00000124260 | MLSDVQSMPK | 2094 | HLA-A*01:01 |
| 3828 | MAGEA10 | ENSG00000124260 | MLSDVQSMPK | 2094 | HLA-A*03:01 |
| 3829 | MAGEA10 | ENSG00000124260 | MLSDVQSMPK | 2094 | HLA-A*03:02 |
| 3830 | MAGEA10 | ENSG00000124260 | MLSDVQSMPK | 2094 | HLA-A*11:01 |
| 3831 | MAGEA10 | ENSG00000124260 | MLSDVQSMPK | 2094 | HLA-A*68:01 |
| 3832 | MAGEA10 | ENSG00000124260 | MLSDVQSMPK | 2094 | HLA-B*27:02 |
| 3833 | MAGEA10 | ENSG00000124260 | MLSDVQSMPK | 2094 | HLA-B*27:05 |
| 3834 | MAGEA10 | ENSG00000124260 | MLSDVQSMPK | 2094 | HLA-C*04:01 |
| 3835 | MAGEA10 | ENSG00000124260 | MLSDVQSMPK | 2094 | HLA-C*06:02 |
| 3836 | MAGEA10 | ENSG00000124260 | MLSDVQSMPK | 2094 | HLA-C*07:01 |
| 3837 | MAGEA10 | ENSG00000124260 | MLSDVQSMPK | 2094 | HLA-C*07:06 |
| 3838 | MAGEA10 | ENSG00000124260 | MLSDVQSM | 2095 | HLA-B*37:01 |
| 3839 | MAGEA10 | ENSG00000124260 | MPEEDLQSQ | 2096 | HLA-B*35:01 |
| 3840 | MAGEA10 | ENSG00000124260 | MPEEDLQSQ | 2096 | HLA-B*35:03 |
| 3841 | MAGEA10 | ENSG00000124260 | MPEEDLQSQ | 2096 | HLA-B*55:01 |
| 3842 | MAGEA10 | ENSG00000124260 | MPEEDLQSQ | 2096 | HLA-C*03:03 |
| 3843 | MAGEA10 | ENSG00000124260 | MPKTGILIL | 2097 | HLA-B*07:02 |
| 3844 | MAGEA10 | ENSG00000124260 | MPKTGILIL | 2097 | HLA-B*08:01 |
| 3845 | MAGEA10 | ENSG00000124260 | MPKTGILIL | 2097 | HLA-B*35:01 |
| 3846 | MAGEA10 | ENSG00000124260 | MPKTGILIL | 2097 | HLA-B*35:03 |
| 3847 | MAGEA10 | ENSG00000124260 | MPKTGILIL | 2097 | HLA-B*51:01 |
| 3848 | MAGEA10 | ENSG00000124260 | MPKTGILIL | 2097 | HLA-B*54:01 |
| 3849 | MAGEA10 | ENSG00000124260 | MPKTGILIL | 2097 | HLA-C*07:02 |
| 3850 | MAGEA10 | ENSG00000124260 | MPKTGILI | 2098 | HLA-B*08:01 |
| 3851 | MAGEA10 | ENSG00000124260 | MPKTGILI | 2098 | HLA-B*51:01 |
| 3852 | MAGEA10 | ENSG00000124260 | MPKTGILI | 2098 | HLA-B*54:01 |
| 3853 | MAGEA10 | ENSG00000124260 | NGSDPRSFPL | 2099 | HLA-C*16:01 |
| 3854 | MAGEA10 | ENSG00000124260 | NGSDPRSF | 2100 | HLA-C*16:01 |
| 3855 | MAGEA10 | ENSG00000124260 | NPPQSAQI | 2101 | HLA-B*51:01 |
| 3856 | MAGEA10 | ENSG00000124260 | NYEDHFPLLF | 2102 | HLA-A*24:02 |
| 3857 | MAGEA10 | ENSG00000124260 | NYEDHFPLLF | 2102 | HLA-A*29:02 |
| 3858 | MAGEA10 | ENSG00000124260 | NYEDHFPLL | 2103 | HLA-A*23:01 |
| 3859 | MAGEA10 | ENSG00000124260 | NYEDHFPLL | 2103 | HLA-A*24:02 |
| 3860 | MAGEA10 | ENSG00000124260 | PDSESLPR | 2104 | HLA-B*27:02 |
| 3861 | MAGEA10 | ENSG00000124260 | PLIPSTPEEV | 2105 | HLA-A*02:03 |
| 3862 | MAGEA10 | ENSG00000124260 | QDWVQENYL | 2106 | HLA-A*30:01 |
| 3863 | MAGEA10 | ENSG00000124260 | QIACSSPSV | 2107 | HLA-A*02:01 |
| 3864 | MAGEA10 | ENSG00000124260 | QKEESPSTL | 2108 | HLA-B*39:01 |
| 3865 | MAGEA10 | ENSG00000124260 | QMKEPITKA | 2109 | HLA-A*02:03 |
| 3866 | MAGEA10 | ENSG00000124260 | QMKEPITKA | 2109 | HLA-B*55:01 |
| 3867 | MAGEA10 | ENSG00000124260 | QSDEGSSSQK | 2110 | HLA-A*01:01 |
| 3868 | MAGEA10 | ENSG00000124260 | QSDEGSSSQ | 2111 | HLA-C*05:01 |
| 3869 | MAGEA10 | ENSG00000124260 | QSNIPKTGIL | 2112 | HLA-B*08:01 |
| 3870 | MAGEA10 | ENSG00000124260 | QSNIPKTGIL | 2112 | HLA-B*58:01 |
| 3871 | MAGEA10 | ENSG00000124260 | QSNPKTGI | 2113 | HLA-B*08:01 |
| 3872 | MAGEA10 | ENSG00000124260 | QVLPDSESLPR | 2114 | HLA-A*03:01 |
| 3873 | MAGEA10 | ENSG00000124260 | QVLPDSESLPR | 2114 | HLA-A*11:01 |
| 3874 | MAGEA10 | ENSG00000124260 | QVLPDSESLPR | 2114 | HLA-A*31:01 |
| 3875 | MAGEA10 | ENSG00000124260 | QVLPDSESLPR | 2114 | HLA-A*33:01 |
| 3876 | MAGEA10 | ENSG00000124260 | QVLPDSESLPR | 2114 | HLA-A*33:03 |
| 3877 | MAGEA10 | ENSG00000124260 | QVLPDSESLPR | 2114 | HLA-A*68:01 |
| 3878 | MAGEA10 | ENSG00000124260 | QVLPDSESLPR | 2114 | HLA-B*27:02 |
| 3879 | MAGEA10 | ENSG00000124260 | QVLPDSESLPR | 2114 | HLA-C*07:06 |
| 3880 | MAGEA10 | ENSG00000124260 | QVLPDSESL | 2115 | HLA-B*15:01 |
| 3881 | MAGEA10 | ENSG00000124260 | QVLPDSESL | 2115 | HLA-B*35:01 |
| 3882 | MAGEA10 | ENSG00000124260 | QVLPDSESL | 2115 | HLA-B*35:03 |
| 3883 | MAGEA10 | ENSG00000124260 | QVLPDSESL | 2115 | HLA-B*38:01 |
| 3884 | MAGEA10 | ENSG00000124260 | QVLPDSESL | 2115 | HLA-B*39:01 |
| 3885 | MAGEA10 | ENSG00000124260 | QVLPDSESL | 2115 | HLA-C*01:02 |
| 3886 | MAGEA10 | ENSG00000124260 | QVLPDSESL | 2115 | HLA-C*03:03 |
| 3887 | MAGEA10 | ENSG00000124260 | QVLPDSESL | 2115 | HLA-C*03:04 |
| 3888 | MAGEA10 | ENSG00000124260 | QVPGSDPARY | 2116 | HLA-A*01:01 |
| 3889 | MAGEA10 | ENSG00000124260 | QVPGSDPARY | 2116 | HLA-A*25:01 |
| 3890 | MAGEA10 | ENSG00000124260 | QVPGSDPARY | 2116 | HLA-A*26:01 |
| 3891 | MAGEA10 | ENSG00000124260 | QVPGSDPARY | 2116 | HLA-A*29:02 |
| 3892 | MAGEA10 | ENSG00000124260 | QVPGSDPARY | 2116 | HLA-A*30:02 |
| 3893 | MAGEA10 | ENSG00000124260 | QVPGSDPARY | 2116 | HLA-C*07:04 |
| 3894 | MAGEA10 | ENSG00000124260 | RIATTDDTTAM | 2117 | HLA-B*15:01 |
| 3895 | MAGEA10 | ENSG00000124260 | RNYEDHFPLLF | 2118 | HLA-B*57:01 |
| 3896 | MAGEA10 | ENSG00000124260 | RQVPGSDPARY | 65 | HLA-A*29:02 |
| 3897 | MAGEA10 | ENSG00000124260 | RQVPGSDPARY | 65 | HLA-A*30:02 |
| 3898 | MAGEA10 | ENSG00000124260 | RQVPGSDPARY | 65 | HLA-A*32:01 |
| 3899 | MAGEA10 | ENSG00000124260 | RQVPGSDPARY | 65 | HLA-B*15:01 |
| 3900 | MAGEA10 | ENSG00000124260 | RQVPGSDPARY | 65 | HLA-B*15:03 |
| 3901 | MAGEA10 | ENSG00000124260 | RQVPGSDPARY | 65 | HLA-B*57:01 |
| 3902 | MAGEA10 | ENSG00000124260 | RQVPGSDPARY | 65 | HLA-B*58:01 |
| 3903 | MAGEA10 | ENSG00000124260 | SASSSATGSF | 2119 | HLA-A*25:01 |
| 3904 | MAGEA10 | ENSG00000124260 | SASSSATGSF | 2119 | HLA-A*26:01 |
| 3905 | MAGEA10 | ENSG00000124260 | SASSSATGSF | 2119 | HLA-A*30:02 |
| 3906 | MAGEA10 | ENSG00000124260 | SASSSATGSF | 2119 | HLA-B*07:02 |
| 3907 | MAGEA10 | ENSG00000124260 | SASSSATGSF | 2119 | HLA-C*03:04 |
| 3908 | MAGEA10 | ENSG00000124260 | SATGSFSYP | 2120 | HLA-C*16:02 |
| 3909 | MAGEA10 | ENSG00000124260 | SATGSFSY | 2121 | HLA-A*30:02 |
| 3910 | MAGEA10 | ENSG00000124260 | SATGSFSY | 2121 | HLA-B*15:01 |
| 3911 | MAGEA10 | ENSG00000124260 | SATGSFSY | 2121 | HLA-B*15:03 |
| 3912 | MAGEA10 | ENSG00000124260 | SATGSFSY | 2121 | HLA-B*35:01 |
| 3913 | MAGEA10 | ENSG00000124260 | SATGSFSY | 2121 | HLA-B*39:01 |
| 3914 | MAGEA10 | ENSG00000124260 | SATGSFSY | 2121 | HLA-B*58:01 |
| 3915 | MAGEA10 | ENSG00000124260 | SATGSFSY | 2121 | HLA-C*16:01 |
| 3916 | MAGEA10 | ENSG00000124260 | SATGSFSY | 2121 | HLA-C*16:02 |
| 3917 | MAGEA10 | ENSG00000124260 | SDPARYEF | 2122 | HLA-B*37:01 |
| 3918 | MAGEA10 | ENSG00000124260 | SDPRSFPLW | 2123 | HLA-A*24:02 |
| 3919 | MAGEA10 | ENSG00000124260 | SDVQSMPK | 2124 | HLA-C*06:02 |
| 3920 | MAGEA10 | ENSG00000124260 | SEASECMLLV | 2125 | HLA-B*49:01 |
| 3921 | MAGEA10 | ENSG00000124260 | SEASECMLL | 2126 | HLA-A*30:01 |
| 3922 | MAGEA10 | ENSG00000124260 | SEASECMLL | 2126 | HLA-B*40:01 |
| 3923 | MAGEA10 | ENSG00000124260 | SEASECMLL | 2126 | HLA-B*44:03 |
| 3924 | MAGEA10 | ENSG00000124260 | SEASECMLL | 2126 | HLA-B*49:01 |
| 3925 | MAGEA10 | ENSG00000124260 | SECMLLVF | 2127 | HLA-B*18:01 |
| 3926 | MAGEA10 | ENSG00000124260 | SEIDEKVTDLV | 2128 | HLA-B*49:01 |
| 3927 | MAGEA10 | ENSG00000124260 | SEIDEKVTDL | 2129 | HLA-A*30:01 |
| 3928 | MAGEA10 | ENSG00000124260 | SEIDEKVTDL | 2129 | HLA-B*40:01 |
| 3929 | MAGEA10 | ENSG00000124260 | SEIDEKVTDL | 2129 | HLA-B*40:02 |
| 3930 | MAGEA10 | ENSG00000124260 | SEIDEKVTDL | 2129 | HLA-B*44:02 |
| 3931 | MAGEA10 | ENSG00000124260 | SEIDEKVTDL | 2129 | HLA-B*44:03 |
| 3932 | MAGEA10 | ENSG00000124260 | SEIDEKVTDL | 2129 | HLA-B*49:01 |
| 3933 | MAGEA10 | ENSG00000124260 | SESLPRSEI | 2130 | HLA-B*37:01 |
| 3934 | MAGEA10 | ENSG00000124260 | SESLPRSEI | 2130 | HLA-B*40:01 |
| 3935 | MAGEA10 | ENSG00000124260 | SESLPRSEI | 2130 | HLA-B*44:02 |
| 3936 | MAGEA10 | ENSG00000124260 | SESLPRSEI | 2130 | HLA-B*44:03 |
| 3937 | MAGEA10 | ENSG00000124260 | SESLPRSEI | 2130 | HLA-B*49:01 |
| 3938 | MAGEA10 | ENSG00000124260 | SFVLVTSL | 2131 | HLA-A*23:01 |
| 3939 | MAGEA10 | ENSG00000124260 | SFVLVTSL | 2131 | HLA-C*14:02 |
| 3940 | MAGEA10 | ENSG00000124260 | SIVFIEGY | 2132 | HLA-B*15:01 |
| 3941 | MAGEA10 | ENSG00000124260 | SLLKFLAKV | 2133 | HLA-A*02:01 |
| 3942 | MAGEA10 | ENSG00000124260 | SLLKFLAKV | 2133 | HLA-A*02:03 |
| 3943 | MAGEA10 | ENSG00000124260 | SLLKFLAKV | 2133 | HLA-A*02:04 |
| 3944 | MAGEA10 | ENSG00000124260 | SLLKFLAKV | 2133 | HLA-A*02:07 |
| 3945 | MAGEA10 | ENSG00000124260 | SLLKFLAK | 2134 | HLA-A*03:01 |
| 3946 | MAGEA10 | ENSG00000124260 | SPSTLQVL | 2135 | HLA-B*07:02 |
| 3947 | MAGEA10 | ENSG00000124260 | SPSVVASLPL | 2136 | HLA-B*07:02 |
| 3948 | MAGEA10 | ENSG00000124260 | SPSVVASLPL | 2136 | HLA-C*07:02 |
| 3949 | MAGEA10 | ENSG00000124260 | SPSVVASL | 2137 | HLA-B*07:02 |
| 3950 | MAGEA10 | ENSG00000124260 | SPSVVASL | 2137 | HLA-B*08:01 |
| 3951 | MAGEA10 | ENSG00000124260 | SPSVVASL | 2137 | HLA-B*37:01 |
| 3952 | MAGEA10 | ENSG00000124260 | SPSVVASL | 2137 | HLA-B*56:01 |
| 3953 | MAGEA10 | ENSG00000124260 | SPSVVASL | 2137 | HLA-C*07:02 |
| 3954 | MAGEA10 | ENSG00000124260 | SQKEESPSTL | 2138 | HLA-B*15:01 |
| 3955 | MAGEA10 | ENSG00000124260 | SSATGSFSY | 2139 | HLA-A*01:01 |
| 3956 | MAGEA10 | ENSG00000124260 | SSATGSFSY | 2139 | HLA-A*11:01 |
| 3957 | MAGEA10 | ENSG00000124260 | SSATGSFSY | 2139 | HLA-A*25:01 |
| 3958 | MAGEA10 | ENSG00000124260 | SSATGSFSY | 2139 | HLA-A*26:01 |
| 3959 | MAGEA10 | ENSG00000124260 | SSATGSFSY | 2139 | HLA-A*29:02 |
| 3960 | MAGEA10 | ENSG00000124260 | SSATGSFSY | 2139 | HLA-A*30:02 |
| 3961 | MAGEA10 | ENSG00000124260 | SSATGSFSY | 2139 | HLA-A*32:01 |
| 3962 | MAGEA10 | ENSG00000124260 | SSATGSFSY | 2139 | HLA-B*15:01 |
| 3963 | MAGEA10 | ENSG00000124260 | SSATGSFSY | 2139 | HLA-B*15:03 |
| 3964 | MAGEA10 | ENSG00000124260 | SSATGSFSY | 2139 | HLA-B*27:05 |
| 3965 | MAGEA10 | ENSG00000124260 | SSATGSFSY | 2139 | HLA-B*35:01 |
| 3966 | MAGEA10 | ENSG00000124260 | SSATGSFSY | 2139 | HLA-B*46:01 |
| 3967 | MAGEA10 | ENSG00000124260 | SSATGSFSY | 2139 | HLA-B*58:01 |
| 3968 | MAGEA10 | ENSG00000124260 | SSATGSFSY | 2139 | HLA-C*02:02 |
| 3969 | MAGEA10 | ENSG00000124260 | SSATGSFSY | 2139 | HLA-C*07:01 |
| 3970 | MAGEA10 | ENSG00000124260 | SSATGSFSY | 2139 | HLA-C*07:06 |
| 3971 | MAGEA10 | ENSG00000124260 | SSATGSFSY | 2139 | HLA-C*16:01 |
| 3972 | MAGEA10 | ENSG00000124260 | SSATGSFSY | 2139 | HLA-C*16:02 |
| 3973 | MAGEA10 | ENSG00000124260 | SSATGSFSY | 2139 | HLA-C*16:04 |
| 3974 | MAGEA10 | ENSG00000124260 | SSPSVVASL | 66 | HLA-A*02:07 |
| 3975 | MAGEA10 | ENSG00000124260 | SSPSVVASL | 66 | HLA-A*24:02 |
| 3976 | MAGEA10 | ENSG00000124260 | SSPSVVASL | 66 | HLA-A*25:01 |
| 3977 | MAGEA10 | ENSG00000124260 | SSPSVVASL | 66 | HLA-A*26:01 |
| 3978 | MAGEA10 | ENSG00000124260 | SSPSVVASL | 66 | HLA-B*46:01 |
| 3979 | MAGEA10 | ENSG00000124260 | SSPSVVASL | 66 | HLA-B*58:01 |
| 3980 | MAGEA10 | ENSG00000124260 | SSPSVVASL | 66 | HLA-C*01:02 |
| 3981 | MAGEA10 | ENSG00000124260 | SSPSVVASL | 66 | HLA-C*03:03 |
| 3982 | MAGEA10 | ENSG00000124260 | SSPSVVASL | 66 | HLA-C*03:04 |
| 3983 | MAGEA10 | ENSG00000124260 | SSPSVVASL | 66 | HLA-C*14:02 |
| 3984 | MAGEA10 | ENSG00000124260 | SSSATGSFSY | 2140 | HLA-A*01:01 |
| 3985 | MAGEA10 | ENSG00000124260 | SSSATGSFSY | 2140 | HLA-A*29:02 |
| 3986 | MAGEA10 | ENSG00000124260 | SSSATGSFSY | 2140 | HLA-A*30:02 |
| 3987 | MAGEA10 | ENSG00000124260 | SSSATGSF | 2141 | HLA-C*05:01 |
| 3988 | MAGEA10 | ENSG00000124260 | SSSFPSSF | 2142 | HLA-B*37:01 |
| 3989 | MAGEA10 | ENSG00000124260 | SSSSSSCY | 2143 | HLA-A*30:02 |
| 3990 | MAGEA10 | ENSG00000124260 | SSSSSSSCY | 2144 | HLA-A*01:01 |
| 3991 | MAGEA10 | ENSG00000124260 | SSSSSSSCY | 2144 | HLA-A*26:01 |
| 3992 | MAGEA10 | ENSG00000124260 | SSSSSSSCY | 2144 | HLA-A*30:02 |
| 3993 | MAGEA10 | ENSG00000124260 | SSSSSSSCY | 2144 | HLA-B*15:01 |
| 3994 | MAGEA10 | ENSG00000124260 | SSSSSSSCY | 2144 | HLA-B*15:03 |
| 3995 | MAGEA10 | ENSG00000124260 | SSSSSSSCY | 2144 | HLA-C*07:04 |
| 3996 | MAGEA10 | ENSG00000124260 | SSSSSSSCY | 2144 | HLA-C*16:01 |
| 3997 | MAGEA10 | ENSG00000124260 | SSSSSSSSCY | 2145 | HLA-A*30:02 |
| 3998 | MAGEA10 | ENSG00000124260 | SSSSSSSSSCY | 2146 | HLA-A*30:02 |
| 3999 | MAGEA10 | ENSG00000124260 | SSTSTSSSF | 2147 | HLA-A*25:01 |
| 4000 | MAGEA10 | ENSG00000124260 | SSTSTSSSF | 2147 | HLA-A*26:01 |
| 4001 | MAGEA10 | ENSG00000124260 | SSTSTSSSF | 2147 | HLA-A*30:02 |
| 4002 | MAGEA10 | ENSG00000124260 | SSTSTSSSF | 2147 | HLA-A*32:01 |
| 4003 | MAGEA10 | ENSG00000124260 | SSTSTSSSF | 2147 | HLA-B*15:01 |
| 4004 | MAGEA10 | ENSG00000124260 | SSTSTSSSF | 2147 | HLA-B*15:03 |
| 4005 | MAGEA10 | ENSG00000124260 | SSTSTSSSF | 2147 | HLA-B*46:01 |
| 4006 | MAGEA10 | ENSG00000124260 | SSTSTSSSF | 2147 | HLA-B*58:01 |
| 4007 | MAGEA10 | ENSG00000124260 | SSTSTSSSF | 2147 | HLA-C*02:02 |
| 4008 | MAGEA10 | ENSG00000124260 | SSTSTSSSF | 2147 | HLA-C*03:03 |
| 4009 | MAGEA10 | ENSG00000124260 | SSTSTSSSF | 2147 | HLA-C*03:04 |
| 4010 | MAGEA10 | ENSG00000124260 | SSTSTSSSF | 2147 | HLA-C*14:02 |
| 4011 | MAGEA10 | ENSG00000124260 | SSTSTSSSF | 2147 | HLA-C*16:01 |
| 4012 | MAGEA10 | ENSG00000124260 | SSTSTSSSF | 2147 | HLA-C*16:02 |
| 4013 | MAGEA10 | ENSG00000124260 | SSTSTSSSF | 2147 | HLA-C*16:04 |
| 4014 | MAGEA10 | ENSG00000124260 | STSSSFPSSF | 2148 | HLA-A*25:01 |
| 4015 | MAGEA10 | ENSG00000124260 | STSSSFPSSF | 2148 | HLA-A*26:01 |
| 4016 | MAGEA10 | ENSG00000124260 | STSTSSSF | 2149 | HLA-C*05:01 |
| 4017 | MAGEA10 | ENSG00000124260 | TAMASASSSA | 2150 | HLA-B*54:01 |
| 4018 | MAGEA10 | ENSG00000124260 | TAMASASSSA | 2150 | HLA-B*56:01 |
| 4019 | MAGEA10 | ENSG00000124260 | TDLVQFLL | 2151 | HLA-B*37:01 |
| 4020 | MAGEA10 | ENSG00000124260 | TKAEILESV | 2152 | HLA-A*68:02 |
| 4021 | MAGEA10 | ENSG00000124260 | TPEEVIWEAL | 2153 | HLA-B*35:03 |
| 4022 | MAGEA10 | ENSG00000124260 | TPEEVIWEA | 2154 | HLA-B*35:03 |
| 4023 | MAGEA10 | ENSG00000124260 | TPEEVIWEA | 2154 | HLA-B*54:01 |
| 4024 | MAGEA10 | ENSG00000124260 | TPEEVIWEA | 2154 | HLA-B*56:01 |
| 4025 | MAGEA10 | ENSG00000124260 | TPNPPQSAQIA | 2155 | HLA-B*54:01 |
| 4026 | MAGEA10 | ENSG00000124260 | TPNPPQSAQIA | 2155 | HLA-B*56:01 |
| 4027 | MAGEA10 | ENSG00000124260 | TPNPPQSAQI | 2156 | HLA-B*07:02 |
| 4028 | MAGEA10 | ENSG00000124260 | TPNPPQSAQI | 2156 | HLA-B*51:01 |
| 4029 | MAGEA10 | ENSG00000124260 | TPNPPQSAQI | 2156 | HLA-B*56:01 |
| 4030 | MAGEA10 | ENSG00000124260 | TPNPPQSAQI | 2156 | HLA-C*07:02 |
| 4031 | MAGEA10 | ENSG00000124260 | TPNPPQSA | 2157 | HLA-B*56:01 |
| 4032 | MAGEA10 | ENSG00000124260 | TQDWVQENYL | 2158 | HLA-B*38:01 |
| 4033 | MAGEA10 | ENSG00000124260 | TQDWVQENYL | 2158 | HLA-C*05:01 |
| 4034 | MAGEA10 | ENSG00000124260 | TQDWVQENY | 2159 | HLA-A*01:01 |
| 4035 | MAGEA10 | ENSG00000124260 | TQDWVQENY | 2159 | HLA-A*30:02 |
| 4036 | MAGEA10 | ENSG00000124260 | TQDWVQENY | 2159 | HLA-B*15:01 |
| 4037 | MAGEA10 | ENSG00000124260 | TQDWVQENY | 2159 | HLA-B*38:01 |
| 4038 | MAGEA10 | ENSG00000124260 | TQGLEGAQAPL | 2160 | HLA-B*27:05 |
| 4039 | MAGEA10 | ENSG00000124260 | TQGLEGAQAPL | 2160 | HLA-B*38:01 |
| 4040 | MAGEA10 | ENSG00000124260 | TQGLEGAQAPL | 2160 | HLA-B*39:01 |
| 4041 | MAGEA10 | ENSG00000124260 | TSSSFPSSF | 2161 | HLA-A*25:01 |
| 4042 | MAGEA10 | ENSG00000124260 | TSSSFPSSF | 2161 | HLA-A*26:01 |
| 4043 | MAGEA10 | ENSG00000124260 | TSSSFPSSF | 2161 | HLA-A*30:02 |
| 4044 | MAGEA10 | ENSG00000124260 | TSSSFPSSF | 2161 | HLA-A*32:01 |
| 4045 | MAGEA10 | ENSG00000124260 | TSSSFPSSF | 2161 | HLA-B*37:01 |
| 4046 | MAGEA10 | ENSG00000124260 | TSSSFPSSF | 2161 | HLA-C*02:02 |
| 4047 | MAGEA10 | ENSG00000124260 | TSSSFPSSF | 2161 | HLA-C*16:01 |
| 4048 | MAGEA10 | ENSG00000124260 | TSSSFPSSF | 2161 | HLA-C*16:04 |
| 4049 | MAGEA10 | ENSG00000124260 | TTDDTTAMA | 2162 | HLA-A*01:01 |
| 4050 | MAGEA10 | ENSG00000124260 | TTDDTTAMA | 2162 | HLA-C*05:01 |
| 4051 | MAGEA10 | ENSG00000124260 | TTDDTTAM | 2163 | HLA-B*35:03 |
| 4052 | MAGEA10 | ENSG00000124260 | TTDDTTAM | 2163 | HLA-C*04:01 |
| 4053 | MAGEA10 | ENSG00000124260 | TTDDTTAM | 2163 | HLA-C*05:01 |
| 4054 | MAGEA10 | ENSG00000124260 | TTDDTTAM | 2163 | HLA-C*07:04 |
| 4055 | MAGEA10 | ENSG00000124260 | TYDGMLSDVQS | 2164 | HLA-C*04:01 |
| 4056 | MAGEA10 | ENSG00000124260 | TYDGMLSDV | 226 | HLA-B*35:01 |
| 4057 | MAGEA10 | ENSG00000124260 | TYDGMLSDV | 226 | HLA-B*35:03 |
| 4058 | MAGEA10 | ENSG00000124260 | TYDGMLSDV | 226 | HLA-C*04:01 |
| 4059 | MAGEA10 | ENSG00000124260 | TYDGMLSDV | 226 | HLA-C*05:01 |
| 4060 | MAGEA10 | ENSG00000124260 | TYDGMLSDV | 226 | HLA-C*07:04 |
| 4061 | MAGEA10 | ENSG00000124260 | VDPTGHSF | 2165 | HLA-B*37:01 |
| 4062 | MAGEA10 | ENSG00000124260 | VDPTGHSF | 2165 | HLA-C*01:02 |
| 4063 | MAGEA10 | ENSG00000124260 | VDPTGHSF | 2165 | HLA-C*04:01 |
| 4064 | MAGEA10 | ENSG00000124260 | VDPTGHSF | 2165 | HLA-C*07:01 |
| 4065 | MAGEA10 | ENSG00000124260 | VDPTGHSF | 2165 | HLA-C*14:02 |
| 4066 | MAGEA10 | ENSG00000124260 | VDPTGHSF | 2165 | HLA-C*16:01 |
| 4067 | MAGEA10 | ENSG00000124260 | VEEDASSSTST | 2166 | HLA-A*30:01 |
| 4068 | MAGEA10 | ENSG00000124260 | VKEVDPTGHSF | 2167 | HLA-B*15:03 |
| 4069 | MAGEA10 | ENSG00000124260 | VLPDSESLPRS | 2168 | HLA-A*02:07 |
| 4070 | MAGEA10 | ENSG00000124260 | VLPDSESL | 2169 | HLA-C*01:02 |
| 4071 | MAGEA10 | ENSG00000124260 | VLVTSLGLTY | 2170 | HLA-A*01:01 |
| 4072 | MAGEA10 | ENSG00000124260 | VLVTSLGLTY | 2170 | HLA-A*23:01 |
| 4073 | MAGEA10 | ENSG00000124260 | VLVTSLGLTY | 2170 | HLA-A*25:01 |
| 4074 | MAGEA10 | ENSG00000124260 | VLVTSLGLTY | 2170 | HLA-A*26:01 |
| 4075 | MAGEA10 | ENSG00000124260 | VLVTSLGLTY | 2170 | HLA-A*29:02 |
| 4076 | MAGEA10 | ENSG00000124260 | VLVTSLGLTY | 2170 | HLA-A*30:02 |
| 4077 | MAGEA10 | ENSG00000124260 | VLVTSLGLTY | 2170 | HLA-A*32:01 |
| 4078 | MAGEA10 | ENSG00000124260 | VLVTSLGLTY | 2170 | HLA-B*15:01 |
| 4079 | MAGEA10 | ENSG00000124260 | VLVTSLGLTY | 2170 | HLA-B*15:03 |
| 4080 | MAGEA10 | ENSG00000124260 | VLVTSLGLTY | 2170 | HLA-B*46:01 |
| 4081 | MAGEA10 | ENSG00000124260 | VLVTSLGLTY | 2170 | HLA-B*58:01 |
| 4082 | MAGEA10 | ENSG00000124260 | VLVTSLGLTY | 2170 | HLA-C*02:02 |
| 4083 | MAGEA10 | ENSG00000124260 | VLVTSLGLTY | 2170 | HLA-C*07:04 |
| 4084 | MAGEA10 | ENSG00000124260 | VNGSDPRSF | 2171 | HLA-C*16:01 |
| 4085 | MAGEA10 | ENSG00000124260 | VPGSDPARYEF | 2172 | HLA-B*35:01 |
| 4086 | MAGEA10 | ENSG00000124260 | VPGSDPARYEF | 2172 | HLA-B*55:01 |
| 4087 | MAGEA10 | ENSG00000124260 | VPGSDPARY | 2173 | HLA-A*30:02 |
| 4088 | MAGEA10 | ENSG00000124260 | VPGSDPARY | 2173 | HLA-B*35:01 |
| 4089 | MAGEA10 | ENSG00000124260 | VPGSDPARY | 2173 | HLA-B*55:01 |
| 4090 | MAGEA10 | ENSG00000124260 | VPGSDPARY | 2173 | HLA-C*03:03 |
| 4091 | MAGEA10 | ENSG00000124260 | VQENYLEY | 2174 | HLA-A*01:01 |
| 4092 | MAGEA10 | ENSG00000124260 | VQENYLEY | 2174 | HLA-B*39:01 |
| 4093 | MAGEA10 | ENSG00000124260 | VQSMPKTGI | 2175 | HLA-A*32:01 |
| 4094 | MAGEA10 | ENSG00000124260 | VQSMPKTGI | 2175 | HLA-B*13:02 |
| 4095 | MAGEA10 | ENSG00000124260 | VQSMPKTGI | 2175 | HLA-B*38:01 |
| 4096 | MAGEA10 | ENSG00000124260 | VQSMPKTGI | 2175 | HLA-C*06:02 |
| 4097 | MAGEA10 | ENSG00000124260 | VQSMPKTGI | 2175 | HLA-C*07:04 |
| 4098 | MAGEA10 | ENSG00000124260 | VTDLVQFLLF | 2176 | HLA-A*01:01 |
| 4099 | MAGEA10 | ENSG00000124260 | VTDLVQFLL | 2177 | HLA-A*01:01 |
| 4100 | MAGEA10 | ENSG00000124260 | VTDLVQFLL | 2177 | HLA-A*02:07 |
| 4101 | MAGEA10 | ENSG00000124260 | VTDLVQFL | 2178 | HLA-A*01:01 |
| 4102 | MAGEA10 | ENSG00000124260 | VTDLVQFL | 2178 | HLA-C*04:01 |
| 4103 | MAGEA10 | ENSG00000124260 | VTDLVQFL | 2178 | HLA-C*05:01 |
| 4104 | MAGEA10 | ENSG00000124260 | VTSLGLTY | 2179 | HLA-A*01:01 |
| 4105 | MAGEA10 | ENSG00000124260 | VTSLGLTY | 2179 | HLA-A*29:02 |
| 4106 | MAGEA10 | ENSG00000124260 | VTSLGLTY | 2179 | HLA-A*32:01 |
| 4107 | MAGEA10 | ENSG00000124260 | VTSLGLTY | 2179 | HLA-B*15:01 |
| 4108 | MAGEA10 | ENSG00000124260 | VTSLGLTY | 2179 | HLA-B*46:01 |
| 4109 | MAGEA10 | ENSG00000124260 | VTSLGLTY | 2179 | HLA-B*58:01 |
| 4110 | MAGEA10 | ENSG00000124260 | VTSLGLTY | 2179 | HLA-C*03:04 |
| 4111 | MAGEA10 | ENSG00000124260 | VTSLGLTY | 2179 | HLA-C*14:02 |
| 4112 | MAGEA10 | ENSG00000124260 | VTSLGLTY | 2179 | HLA-C*16:01 |
| 4113 | MAGEA10 | ENSG00000124260 | WVQENYLEY | 75 | HLA-A*01:01 |
| 4114 | MAGEA10 | ENSG00000124260 | WVQENYLEY | 75 | HLA-A*25:01 |
| 4115 | MAGEA10 | ENSG00000124260 | WVQENYLEY | 75 | HLA-A*26:01 |
| 4116 | MAGEA10 | ENSG00000124260 | WVQENYLEY | 75 | HLA-A*29:02 |
| 4117 | MAGEA10 | ENSG00000124260 | WVQENYLEY | 75 | HLA-A*30:02 |
| 4118 | MAGEA10 | ENSG00000124260 | WVQENYLEY | 75 | HLA-B*15:01 |
| 4119 | MAGEA10 | ENSG00000124260 | WVQENYLEY | 75 | HLA-B*35:01 |
| 4120 | MAGEA10 | ENSG00000124260 | WVQENYLEY | 75 | HLA-C*07:04 |
| 4121 | MAGEA10 | ENSG00000124260 | YDGMEHLI | 2180 | HLA-B*38:01 |
| 4122 | MAGEA10 | ENSG00000124260 | YDGMEHLI | 2180 | HLA-C*07:01 |
| 4123 | MAGEA10 | ENSG00000124260 | YDGMEHLI | 2180 | HLA-C*07:04 |
| 4124 | MAGEA10 | ENSG00000124260 | YEDHFPLLF | 2181 | HLA-A*01:01 |
| 4125 | MAGEA10 | ENSG00000124260 | YEDHFPLLF | 2181 | HLA-A*02:07 |
| 4126 | MAGEA10 | ENSG00000124260 | YEDHFPLLF | 2181 | HLA-A*24:02 |
| 4127 | MAGEA10 | ENSG00000124260 | YEDHFPLLF | 2181 | HLA-A*29:02 |
| 4128 | MAGEA10 | ENSG00000124260 | YEDHFPLLF | 2181 | HLA-B*18:01 |
| 4129 | MAGEA10 | ENSG00000124260 | YEDHFPLLF | 2181 | HLA-B*44:02 |
| 4130 | MAGEA10 | ENSG00000124260 | YEDHFPLLF | 2181 | HLA-B*44:03 |
| 4131 | MAGEA10 | ENSG00000124260 | YEDHFPLLF | 2181 | HLA-B*49:01 |
| 4132 | MAGEA10 | ENSG00000124260 | YEDHFPLLF | 2181 | HLA-B*57:01 |
| 4133 | MAGEA10 | ENSG00000124260 | YEDHFPLL | 2182 | HLA-B*18:01 |
| 4134 | MAGEA10 | ENSG00000124260 | YEDHFPLL | 2182 | HLA-B*49:01 |
| 4135 | MAGEA10 | ENSG00000124260 | YEFLWGPRA | 2183 | HLA-A*02:04 |
| 4136 | MAGEA10 | ENSG00000124260 | YPLIPSTPEEV | 2184 | HLA-A*68:02 |
| 4137 | MAGEA10 | ENSG00000124260 | YPLIPSTPEEV | 2184 | HLA-B*51:01 |
| 4138 | MAGEA10 | ENSG00000124260 | YPLIPSTPEEV | 2184 | HLA-B*54:01 |
| 4139 | MAGEA10 | ENSG00000124260 | YPLIPSTPEEV | 2184 | HLA-B*56:01 |
| 4140 | MAGEA10 | ENSG00000124260 | YQMKEPITKA | 2185 | HLA-A*02:03 |
| 4141 | MAGEA10 | ENSG00000124260 | YQMKEPITK | 2186 | HLA-A*03:01 |
| 4142 | MAGEA10 | ENSG00000124260 | YQMKEPITK | 2186 | HLA-A*03:02 |
| 4143 | MAGEA10 | ENSG00000124260 | YQMKEPITK | 2186 | HLA-A*11:01 |
| 4144 | MAGEA10 | ENSG00000124260 | YQMKEPITK | 2186 | HLA-B*13:02 |
| 4145 | MAGEA10 | ENSG00000124260 | YQMKEPITK | 2186 | HLA-B*27:05 |
| 4146 | MAGEA10 | ENSG00000124260 | YQMKEPITK | 2186 | HLA-B*38:01 |
| 4147 | MAGEA10 | ENSG00000124260 | YQMKEPITK | 2186 | HLA-C*02:02 |
| 4148 | MAGEA10 | ENSG00000124260 | YQMKEPITK | 2186 | HLA-C*03:03 |
| 4149 | MAGEA10 | ENSG00000124260 | YQMKEPITK | 2186 | HLA-C*03:04 |
| 4150 | MAGEA10 | ENSG00000124260 | YQMKEPITK | 2186 | HLA-C*07:04 |
| 4151 | MAGEA10 | ENSG00000124260 | YRQVPGSDPAR | 2187 | HLA-B*27:05 |
| 4152 | MAGEA4 | ENSG00000147381 | AAVSSSSPL | 2188 | HLA-B*07:02 |
| 4153 | MAGEA4 | ENSG00000147381 | AAVSSSSPL | 2188 | HLA-B*15:01 |
| 4154 | MAGEA4 | ENSG00000147381 | AAVSSSSPL | 2188 | HLA-B*35:03 |
| 4155 | MAGEA4 | ENSG00000147381 | AAVSSSSPL | 2188 | HLA-B*39:01 |
| 4156 | MAGEA4 | ENSG00000147381 | AAVSSSSPL | 2188 | HLA-B*40:01 |
| 4157 | MAGEA4 | ENSG00000147381 | AAVSSSSPL | 2188 | HLA-B*46:01 |
| 4158 | MAGEA4 | ENSG00000147381 | AAVSSSSPL | 2188 | HLA-B*58:01 |
| 4159 | MAGEA4 | ENSG00000147381 | AAVSSSSPL | 2188 | HLA-C*01:02 |
| 4160 | MAGEA4 | ENSG00000147381 | AAVSSSSPL | 2188 | HLA-C*02:02 |
| 4161 | MAGEA4 | ENSG00000147381 | AAVSSSSPL | 2188 | HLA-C*03:03 |
| 4162 | MAGEA4 | ENSG00000147381 | AAVSSSSPL | 2188 | HLA-C*03:04 |
| 4163 | MAGEA4 | ENSG00000147381 | AAVSSSSPL | 2188 | HLA-C*07:02 |
| 4164 | MAGEA4 | ENSG00000147381 | AAVSSSSPL | 2188 | HLA-C*12:03 |
| 4165 | MAGEA4 | ENSG00000147381 | AAVSSSSPL | 2188 | HLA-C*14:02 |
| 4166 | MAGEA4 | ENSG00000147381 | AAVSSSSPL | 2188 | HLA-C*16:01 |
| 4167 | MAGEA4 | ENSG00000147381 | AAVSSSSPL | 2188 | HLA-C*16:04 |
| 4168 | MAGEA4 | ENSG00000147381 | AEMLERVIKNY | 9 | HLA-A*30:02 |
| 4169 | MAGEA4 | ENSG00000147381 | AEMLERVIKNY | 9 | HLA-B*27:02 |
| 4170 | MAGEA4 | ENSG00000147381 | AEMLERVIKNY | 9 | HLA-B*44:02 |
| 4171 | MAGEA4 | ENSG00000147381 | AEMLERVIKNY | 9 | HLA-B*44:03 |
| 4172 | MAGEA4 | ENSG00000147381 | AEMLERVIKNY | 9 | HLA-B*57:01 |
| 4173 | MAGEA4 | ENSG00000147381 | AEMLERVIKNY | 9 | HLA-C*16:04 |
| 4174 | MAGEA4 | ENSG00000147381 | AEMLERVIKN | 2189 | HLA-B*44:02 |
| 4175 | MAGEA4 | ENSG00000147381 | AEMLERVI | 2190 | HLA-B*37:01 |
| 4176 | MAGEA4 | ENSG00000147381 | AEMLERVI | 2190 | HLA-B*44:02 |
| 4177 | MAGEA4 | ENSG00000147381 | AEMLERVI | 2190 | HLA-B*44:03 |
| 4178 | MAGEA4 | ENSG00000147381 | AEMLERVI | 2190 | HLA-B*49:01 |
| 4179 | MAGEA4 | ENSG00000147381 | AESLFREAL | 2191 | HLA-A*30:01 |
| 4180 | MAGEA4 | ENSG00000147381 | AESLFREAL | 2191 | HLA-B*07:02 |
| 4181 | MAGEA4 | ENSG00000147381 | AESLFREAL | 2191 | HLA-B*18:01 |
| 4182 | MAGEA4 | ENSG00000147381 | AESLFREAL | 2191 | HLA-B*27:02 |
| 4183 | MAGEA4 | ENSG00000147381 | AESLFREAL | 2191 | HLA-B*37:01 |
| 4184 | MAGEA4 | ENSG00000147381 | AESLFREAL | 2191 | HLA-B*40:01 |
| 4185 | MAGEA4 | ENSG00000147381 | AESLFREAL | 2191 | HLA-B*40:02 |
| 4186 | MAGEA4 | ENSG00000147381 | AESLFREAL | 2191 | HLA-B*44:02 |
| 4187 | MAGEA4 | ENSG00000147381 | AESLFREAL | 2191 | HLA-B*44:03 |
| 4188 | MAGEA4 | ENSG00000147381 | AESLFREAL | 2191 | HLA-B*49:01 |
| 4189 | MAGEA4 | ENSG00000147381 | AESLFREAL | 2191 | HLA-C*16:04 |
| 4190 | MAGEA4 | ENSG00000147381 | AESLFREA | 2192 | HLA-B*37:01 |
| 4191 | MAGEA4 | ENSG00000147381 | AETSYVKVL | 10 | HLA-A*30:01 |
| 4192 | MAGEA4 | ENSG00000147381 | AETSYVKVL | 10 | HLA-B*18:01 |
| 4193 | MAGEA4 | ENSG00000147381 | AETSYVKVL | 10 | HLA-B*27:02 |
| 4194 | MAGEA4 | ENSG00000147381 | AETSYVKVL | 10 | HLA-B*27:05 |
| 4195 | MAGEA4 | ENSG00000147381 | AETSYVKVL | 10 | HLA-B*37:01 |
| 4196 | MAGEA4 | ENSG00000147381 | AETSYVKVL | 10 | HLA-B*40:01 |
| 4197 | MAGEA4 | ENSG00000147381 | AETSYVKVL | 10 | HLA-B*40:02 |
| 4198 | MAGEA4 | ENSG00000147381 | AETSYVKVL | 10 | HLA-B*44:02 |
| 4199 | MAGEA4 | ENSG00000147381 | AETSYVKVL | 10 | HLA-B*44:03 |
| 4200 | MAGEA4 | ENSG00000147381 | AETSYVKVL | 10 | HLA-B*49:01 |
| 4201 | MAGEA4 | ENSG00000147381 | AETSYVKVL | 10 | HLA-C*02:02 |
| 4202 | MAGEA4 | ENSG00000147381 | AETSYVKVL | 10 | HLA-C*07:04 |
| 4203 | MAGEA4 | ENSG00000147381 | AETSYVKVL | 10 | HLA-C*12:03 |
| 4204 | MAGEA4 | ENSG00000147381 | AETSYVKVL | 10 | HLA-C*16:01 |
| 4205 | MAGEA4 | ENSG00000147381 | AETSYVKVL | 10 | HLA-C*16:02 |
| 4206 | MAGEA4 | ENSG00000147381 | AETSYVKVL | 10 | HLA-C*16:04 |
| 4207 | MAGEA4 | ENSG00000147381 | AETSYVKV | 2193 | HLA-A*30:01 |
| 4208 | MAGEA4 | ENSG00000147381 | AETSYVKV | 2193 | HLA-B*37:01 |
| 4209 | MAGEA4 | ENSG00000147381 | AETSYVKV | 2193 | HLA-B*49:01 |
| 4210 | MAGEA4 | ENSG00000147381 | AKELVTKAEM | 2194 | HLA-B*15:03 |
| 4211 | MAGEA4 | ENSG00000147381 | AKELVTKAEM | 2194 | HLA-C*04:01 |
| 4212 | MAGEA4 | ENSG00000147381 | ALAETSYVKVL | 2195 | HLA-A*02:03 |
| 4213 | MAGEA4 | ENSG00000147381 | ALAETSYVKVL | 2195 | HLA-A*02:04 |
| 4214 | MAGEA4 | ENSG00000147381 | ALAETSYVKV | 2196 | HLA-A*02:01 |
| 4215 | MAGEA4 | ENSG00000147381 | ALAETSYVKV | 2196 | HLA-A*02:03 |
| 4216 | MAGEA4 | ENSG00000147381 | ALAETSYVKV | 2196 | HLA-A*02:04 |
| 4217 | MAGEA4 | ENSG00000147381 | ALAETSYVKV | 2196 | HLA-A*02:07 |
| 4218 | MAGEA4 | ENSG00000147381 | ALAETSYVKV | 2196 | HLA-B*55:01 |
| 4219 | MAGEA4 | ENSG00000147381 | ALAETSYVK | 162 | HLA-A*01:01 |
| 4220 | MAGEA4 | ENSG00000147381 | ALAETSYVK | 162 | HLA-A*03:01 |
| 4221 | MAGEA4 | ENSG00000147381 | ALAETSYVK | 162 | HLA-A*03:02 |
| 4222 | MAGEA4 | ENSG00000147381 | ALAETSYVK | 162 | HLA-A*11:01 |
| 4223 | MAGEA4 | ENSG00000147381 | ALAETSYVK | 162 | HLA-A*29:02 |
| 4224 | MAGEA4 | ENSG00000147381 | ALAETSYVK | 162 | HLA-A*68:01 |
| 4225 | MAGEA4 | ENSG00000147381 | ALAETSYVK | 162 | HLA-B*13:02 |
| 4226 | MAGEA4 | ENSG00000147381 | ALAETSYVK | 162 | HLA-B*27:02 |
| 4227 | MAGEA4 | ENSG00000147381 | ALAETSYVK | 162 | HLA-B*27:05 |
| 4228 | MAGEA4 | ENSG00000147381 | ALAETSYVK | 162 | HLA-C*07:06 |
| 4229 | MAGEA4 | ENSG00000147381 | ALAETSYV | 2197 | HLA-A*02:01 |
| 4230 | MAGEA4 | ENSG00000147381 | ALAETSYV | 2197 | HLA-A*02:03 |
| 4231 | MAGEA4 | ENSG00000147381 | ALAETSYV | 2197 | HLA-B*55:01 |
| 4232 | MAGEA4 | ENSG00000147381 | ALGLVGAQA | 2198 | HLA-A*02:01 |
| 4233 | MAGEA4 | ENSG00000147381 | ALGLVGAQA | 2198 | HLA-A*02:03 |
| 4234 | MAGEA4 | ENSG00000147381 | ALGLVGAQA | 2198 | HLA-B*56:01 |
| 4235 | MAGEA4 | ENSG00000147381 | ALLEEEEGV | 11 | HLA-A*02:01 |
| 4236 | MAGEA4 | ENSG00000147381 | ALLEEEEGV | 11 | HLA-A*02:03 |
| 4237 | MAGEA4 | ENSG00000147381 | ALLEEEEGV | 11 | HLA-A*02:04 |
| 4238 | MAGEA4 | ENSG00000147381 | ALLEEEEGV | 11 | HLA-A*02:07 |
| 4239 | MAGEA4 | ENSG00000147381 | ALLEEEEGV | 11 | HLA-B*13:02 |
| 4240 | MAGEA4 | ENSG00000147381 | ALLEEEEGV | 11 | HLA-B*55:01 |
| 4241 | MAGEA4 | ENSG00000147381 | ALPTTISFTCW | 2199 | HLA-A*02:07 |
| 4242 | MAGEA4 | ENSG00000147381 | ALPTTISFTCW | 2199 | HLA-A*24:02 |
| 4243 | MAGEA4 | ENSG00000147381 | ALPTTISF | 2200 | HLA-A*23:01 |
| 4244 | MAGEA4 | ENSG00000147381 | ALPTTISF | 2200 | HLA-B*15:01 |
| 4245 | MAGEA4 | ENSG00000147381 | ALPTTISF | 2200 | HLA-B*37:01 |
| 4246 | MAGEA4 | ENSG00000147381 | ALPTTISF | 2200 | HLA-B*46:01 |
| 4247 | MAGEA4 | ENSG00000147381 | ALPTTISF | 2200 | HLA-C*01:02 |
| 4248 | MAGEA4 | ENSG00000147381 | ALPTTISF | 2200 | HLA-C*14:02 |
| 4249 | MAGEA4 | ENSG00000147381 | ALSNKVDEL | 2201 | HLA-A*02:01 |
| 4250 | MAGEA4 | ENSG00000147381 | ALSNKVDEL | 2201 | HLA-A*02:03 |
| 4251 | MAGEA4 | ENSG00000147381 | ALSNKVDEL | 2201 | HLA-A*02:04 |
| 4252 | MAGEA4 | ENSG00000147381 | ALSNKVDEL | 2201 | HLA-A*02:07 |
| 4253 | MAGEA4 | ENSG00000147381 | ALSNKVDEL | 2201 | HLA-B*55:01 |
| 4254 | MAGEA4 | ENSG00000147381 | ALSNKVDEL | 2201 | HLA-C*01:02 |
| 4255 | MAGEA4 | ENSG00000147381 | ALSNKVDEL | 2201 | HLA-C*16:01 |
| 4256 | MAGEA4 | ENSG00000147381 | ALSNKVDEL | 2201 | HLA-C*16:02 |
| 4257 | MAGEA4 | ENSG00000147381 | APTTEEQEAAV | 2202 | HLA-B*56:01 |
| 4258 | MAGEA4 | ENSG00000147381 | APTTEEQEA | 2203 | HLA-B*35:03 |
| 4259 | MAGEA4 | ENSG00000147381 | APTTEEQEA | 2203 | HLA-B*55:01 |
| 4260 | MAGEA4 | ENSG00000147381 | APTTEEQEA | 2203 | HLA-B*56:01 |
| 4261 | MAGEA4 | ENSG00000147381 | AQAPTTEEQEA | 2204 | HLA-B*27:05 |
| 4262 | MAGEA4 | ENSG00000147381 | AQAPTTEEQ | 2205 | HLA-B*13:02 |
| 4263 | MAGEA4 | ENSG00000147381 | AQAPTTEEQ | 2205 | HLA-B*15:01 |
| 4264 | MAGEA4 | ENSG00000147381 | ASALPTTISF | 2206 | HLA-A*01:01 |
| 4265 | MAGEA4 | ENSG00000147381 | ASALPTTISF | 2206 | HLA-B*46:01 |
| 4266 | MAGEA4 | ENSG00000147381 | ASALPTTISF | 2206 | HLA-B*57:01 |
| 4267 | MAGEA4 | ENSG00000147381 | ASALPTTISF | 2206 | HLA-B*58:01 |
| 4268 | MAGEA4 | ENSG00000147381 | ASALPTTISF | 2206 | HLA-C*01:02 |
| 4269 | MAGEA4 | ENSG00000147381 | ASALPTTISF | 2206 | HLA-C*04:01 |
| 4270 | MAGEA4 | ENSG00000147381 | ASALPTTISF | 2206 | HLA-C*16:01 |
| 4271 | MAGEA4 | ENSG00000147381 | ASALPTTISF | 2206 | HLA-C*16:04 |
| 4272 | MAGEA4 | ENSG00000147381 | ASESLKMIF | 2207 | HLA-A*01:01 |
| 4273 | MAGEA4 | ENSG00000147381 | ASESLKMIF | 2207 | HLA-B*57:01 |
| 4274 | MAGEA4 | ENSG00000147381 | ASESLKMIF | 2207 | HLA-B*58:01 |
| 4275 | MAGEA4 | ENSG00000147381 | ASESLKMIF | 2207 | HLA-C*12:03 |
| 4276 | MAGEA4 | ENSG00000147381 | ASNTYTLVT | 2208 | HLA-A*11:01 |
| 4277 | MAGEA4 | ENSG00000147381 | AVSSSSPLV | 2209 | HLA-A*02:01 |
| 4278 | MAGEA4 | ENSG00000147381 | AVSSSSPLV | 2209 | HLA-A*02:03 |
| 4279 | MAGEA4 | ENSG00000147381 | AVSSSSPLV | 2209 | HLA-A*02:07 |
| 4280 | MAGEA4 | ENSG00000147381 | AVSSSSPLV | 2209 | HLA-A*03:02 |
| 4281 | MAGEA4 | ENSG00000147381 | AVSSSSPLV | 2209 | HLA-A*26:01 |
| 4282 | MAGEA4 | ENSG00000147381 | AVSSSSPLV | 2209 | HLA-A*32:01 |
| 4283 | MAGEA4 | ENSG00000147381 | AVSSSSPLV | 2209 | HLA-B*13:02 |
| 4284 | MAGEA4 | ENSG00000147381 | AVSSSSPLV | 2209 | HLA-B*27:05 |
| 4285 | MAGEA4 | ENSG00000147381 | AVSSSSPLV | 2209 | HLA-B*39:01 |
| 4286 | MAGEA4 | ENSG00000147381 | AVSSSSPLV | 2209 | HLA-B*55:01 |
| 4287 | MAGEA4 | ENSG00000147381 | AVSSSSPLV | 2209 | HLA-C*02:02 |
| 4288 | MAGEA4 | ENSG00000147381 | AVSSSSPLV | 2209 | HLA-C*06:02 |
| 4289 | MAGEA4 | ENSG00000147381 | AVSSSSPLV | 2209 | HLA-C*16:02 |
| 4290 | MAGEA4 | ENSG00000147381 | AVSSSSPL | 2210 | HLA-C*05:01 |
| 4291 | MAGEA4 | ENSG00000147381 | AYPSLREAALL | 2211 | HLA-A*24:02 |
| 4292 | MAGEA4 | ENSG00000147381 | AYPSLREAAL | 2212 | HLA-A*24:02 |
| 4293 | MAGEA4 | ENSG00000147381 | AYPSLREAAL | 2212 | HLA-C*01:02 |
| 4294 | MAGEA4 | ENSG00000147381 | AYPSLREAAL | 2212 | HLA-C*14:02 |
| 4295 | MAGEA4 | ENSG00000147381 | DAESLFREA | 2213 | HLA-B*51:01 |
| 4296 | MAGEA4 | ENSG00000147381 | DAESLFREA | 2213 | HLA-B*54:01 |
| 4297 | MAGEA4 | ENSG00000147381 | DELAHFLLRKY | 2214 | HLA-B*44:02 |
| 4298 | MAGEA4 | ENSG00000147381 | DELAHFLLR | 2215 | HLA-A*33:01 |
| 4299 | MAGEA4 | ENSG00000147381 | DELAHFLL | 2216 | HLA-B*18:01 |
| 4300 | MAGEA4 | ENSG00000147381 | DELAHFLL | 2216 | HLA-B*37:01 |
| 4301 | MAGEA4 | ENSG00000147381 | DELAHFLL | 2216 | HLA-B*40:02 |
| 4302 | MAGEA4 | ENSG00000147381 | DELAHFLL | 2216 | HLA-B*44:02 |
| 4303 | MAGEA4 | ENSG00000147381 | DGLLGNNQI | 2217 | HLA-B*51:01 |
| 4304 | MAGEA4 | ENSG00000147381 | DGREHTVY | 2218 | HLA-B*08:01 |
| 4305 | MAGEA4 | ENSG00000147381 | DGREHTVY | 2218 | HLA-B*18:01 |
| 4306 | MAGEA4 | ENSG00000147381 | DPASNTYTLV | 2219 | HLA-A*68:01 |
| 4307 | MAGEA4 | ENSG00000147381 | DPASNTYTLV | 2219 | HLA-B*51:01 |
| 4308 | MAGEA4 | ENSG00000147381 | DPASNTYTL | 2220 | HLA-A*23:01 |
| 4309 | MAGEA4 | ENSG00000147381 | DPASNTYTL | 2220 | HLA-A*68:01 |
| 4310 | MAGEA4 | ENSG00000147381 | DPASNTYTL | 2220 | HLA-B*07:02 |
| 4311 | MAGEA4 | ENSG00000147381 | DPASNTYTL | 2220 | HLA-B*35:01 |
| 4312 | MAGEA4 | ENSG00000147381 | DPASNTYTL | 2220 | HLA-B*35:03 |
| 4313 | MAGEA4 | ENSG00000147381 | DPASNTYTL | 2220 | HLA-B*38:01 |
| 4314 | MAGEA4 | ENSG00000147381 | DPASNTYTL | 2220 | HLA-B*39:01 |
| 4315 | MAGEA4 | ENSG00000147381 | DPASNTYTL | 2220 | HLA-B*51:01 |
| 4316 | MAGEA4 | ENSG00000147381 | DPASNTYTL | 2220 | HLA-B*55:01 |
| 4317 | MAGEA4 | ENSG00000147381 | DPASNTYTL | 2220 | HLA-B*56:01 |
| 4318 | MAGEA4 | ENSG00000147381 | DPASNTYTL | 2220 | HLA-C*07:02 |
| 4319 | MAGEA4 | ENSG00000147381 | DVKEVDPASN | 2221 | HLA-A*25:01 |
| 4320 | MAGEA4 | ENSG00000147381 | DVKEVDPASN | 2221 | HLA-A*26:01 |
| 4321 | MAGEA4 | ENSG00000147381 | DVKEVDPASN | 2221 | HLA-A*33:01 |
| 4322 | MAGEA4 | ENSG00000147381 | DVKEVDPASN | 2221 | HLA-A*68:01 |
| 4323 | MAGEA4 | ENSG00000147381 | EALGLVGAQA | 2222 | HLA-A*33:03 |
| 4324 | MAGEA4 | ENSG00000147381 | EALGLVGAQA | 2222 | HLA-B*54:01 |
| 4325 | MAGEA4 | ENSG00000147381 | EALGLVGAQA | 2222 | HLA-C*07:06 |
| 4326 | MAGEA4 | ENSG00000147381 | EALGLVGAQ | 2223 | HLA-A*26:01 |
| 4327 | MAGEA4 | ENSG00000147381 | EALGLVGAQ | 2223 | HLA-A*33:01 |
| 4328 | MAGEA4 | ENSG00000147381 | EALGLVGAQ | 2223 | HLA-A*33:03 |
| 4329 | MAGEA4 | ENSG00000147381 | EALGLVGAQ | 2223 | HLA-C*04:01 |
| 4330 | MAGEA4 | ENSG00000147381 | EALGLVGAQ | 2223 | HLA-C*07:01 |
| 4331 | MAGEA4 | ENSG00000147381 | EALGLVGAQ | 2223 | HLA-C*12:03 |
| 4332 | MAGEA4 | ENSG00000147381 | EALGLVGA | 2224 | HLA-B*54:01 |
| 4333 | MAGEA4 | ENSG00000147381 | EAQEEALGL | 2225 | HLA-B*35:03 |
| 4334 | MAGEA4 | ENSG00000147381 | EAQEEALGL | 2225 | HLA-C*07:06 |
| 4335 | MAGEA4 | ENSG00000147381 | EEALGLVGAQA | 2226 | HLA-B*44:03 |
| 4336 | MAGEA4 | ENSG00000147381 | EEALGLVGA | 2227 | HLA-A*30:01 |
| 4337 | MAGEA4 | ENSG00000147381 | EEALGLVGA | 2227 | HLA-B*18:01 |
| 4338 | MAGEA4 | ENSG00000147381 | EEALGLVGA | 2227 | HLA-B*40:02 |
| 4339 | MAGEA4 | ENSG00000147381 | EEALGLVGA | 2227 | HLA-B*49:01 |
| 4340 | MAGEA4 | ENSG00000147381 | EEEGPSTSPDA | 2228 | HLA-B*39:01 |
| 4341 | MAGEA4 | ENSG00000147381 | EEEGPSTSP | 2229 | HLA-B*39:01 |
| 4342 | MAGEA4 | ENSG00000147381 | EEIWEELGV | 2230 | HLA-B*49:01 |
| 4343 | MAGEA4 | ENSG00000147381 | EELGVMGVY | 98 | HLA-A*25:01 |
| 4344 | MAGEA4 | ENSG00000147381 | EELGVMGVY | 98 | HLA-A*26:01 |
| 4345 | MAGEA4 | ENSG00000147381 | EELGVMGVY | 98 | HLA-A*30:02 |
| 4346 | MAGEA4 | ENSG00000147381 | EELGVMGVY | 98 | HLA-B*15:03 |
| 4347 | MAGEA4 | ENSG00000147381 | EELGVMGVY | 98 | HLA-B*18:01 |
| 4348 | MAGEA4 | ENSG00000147381 | EELGVMGVY | 98 | HLA-B*27:02 |
| 4349 | MAGEA4 | ENSG00000147381 | EELGVMGVY | 98 | HLA-B*40:01 |
| 4350 | MAGEA4 | ENSG00000147381 | EELGVMGVY | 98 | HLA-B*44:02 |
| 4351 | MAGEA4 | ENSG00000147381 | EELGVMGVY | 98 | HLA-B*44:03 |
| 4352 | MAGEA4 | ENSG00000147381 | EELGVMGVY | 98 | HLA-C*16:04 |
| 4353 | MAGEA4 | ENSG00000147381 | EELGVMGV | 2231 | HLA-B*18:01 |
| 4354 | MAGEA4 | ENSG00000147381 | EELGVMGV | 2231 | HLA-B*37:01 |
| 4355 | MAGEA4 | ENSG00000147381 | EELGVMGV | 2231 | HLA-B*40:02 |
| 4356 | MAGEA4 | ENSG00000147381 | EELGVMGV | 2231 | HLA-B*49:01 |
| 4357 | MAGEA4 | ENSG00000147381 | EEVPAAESA | 2232 | HLA-A*30:01 |
| 4358 | MAGEA4 | ENSG00000147381 | EEVPAAESA | 2232 | HLA-B*18:01 |
| 4359 | MAGEA4 | ENSG00000147381 | EEVPAAESA | 2232 | HLA-B*40:01 |
| 4360 | MAGEA4 | ENSG00000147381 | EEVPAAESA | 2232 | HLA-B*40:02 |
| 4361 | MAGEA4 | ENSG00000147381 | EEVPAAESA | 2232 | HLA-B*49:01 |
| 4362 | MAGEA4 | ENSG00000147381 | EHTVYGEPR | 2233 | HLA-A*33:01 |
| 4363 | MAGEA4 | ENSG00000147381 | EHTVYGEPR | 2233 | HLA-A*33:03 |
| 4364 | MAGEA4 | ENSG00000147381 | EHVVRVNAR | 2234 | HLA-A*33:03 |
| 4365 | MAGEA4 | ENSG00000147381 | EIWEELGVMGV | 2235 | HLA-A*68:02 |
| 4366 | MAGEA4 | ENSG00000147381 | ELGVMGVY | 2236 | HLA-A*25:01 |
| 4367 | MAGEA4 | ENSG00000147381 | ELGVMGVY | 2236 | HLA-A*26:01 |
| 4368 | MAGEA4 | ENSG00000147381 | ELGVMGVY | 2236 | HLA-B*15:01 |
| 4369 | MAGEA4 | ENSG00000147381 | ELGVMGVY | 2236 | HLA-C*07:01 |
| 4370 | MAGEA4 | ENSG00000147381 | ELGVMGVY | 2236 | HLA-C*07:04 |
| 4371 | MAGEA4 | ENSG00000147381 | ELVTKAEMLER | 2237 | HLA-A*33:01 |
| 4372 | MAGEA4 | ENSG00000147381 | ELVTKAEML | 2238 | HLA-A*25:01 |
| 4373 | MAGEA4 | ENSG00000147381 | ELVTKAEML | 2238 | HLA-A*26:01 |
| 4374 | MAGEA4 | ENSG00000147381 | ELVTKAEML | 2238 | HLA-B*08:01 |
| 4375 | MAGEA4 | ENSG00000147381 | ELVTKAEM | 2239 | HLA-B*08:01 |
| 4376 | MAGEA4 | ENSG00000147381 | EMLERVIKNY | 2240 | HLA-A*25:01 |
| 4377 | MAGEA4 | ENSG00000147381 | EMLERVIKNY | 2240 | HLA-B*44:02 |
| 4378 | MAGEA4 | ENSG00000147381 | ESAGPPQSP | 2241 | HLA-B*39:01 |
| 4379 | MAGEA4 | ENSG00000147381 | ESLFREALSNK | 2242 | HLA-A*33:01 |
| 4380 | MAGEA4 | ENSG00000147381 | ESLFREAL | 2243 | HLA-B*08:01 |
| 4381 | MAGEA4 | ENSG00000147381 | ETSYVKVLEHV | 2244 | HLA-A*68:02 |
| 4382 | MAGEA4 | ENSG00000147381 | ETSYVKVLEH | 2245 | HLA-A*68:01 |
| 4383 | MAGEA4 | ENSG00000147381 | ETSYVKVLE | 2246 | HLA-A*68:02 |
| 4384 | MAGEA4 | ENSG00000147381 | EVDPASNTYTL | 2247 | HLA-A*01:01 |
| 4385 | MAGEA4 | ENSG00000147381 | EVDPASNTYTL | 2247 | HLA-A*02:07 |
| 4386 | MAGEA4 | ENSG00000147381 | EVDPASNTYTL | 2247 | HLA-A*25:01 |
| 4387 | MAGEA4 | ENSG00000147381 | EVDPASNTYTL | 2247 | HLA-A*26:01 |
| 4388 | MAGEA4 | ENSG00000147381 | EVDPASNTYTL | 2247 | HLA-A*33:03 |
| 4389 | MAGEA4 | ENSG00000147381 | EVDPASNTYTL | 2247 | HLA-A*68:01 |
| 4390 | MAGEA4 | ENSG00000147381 | EVDPASNTYTL | 2247 | HLA-A*68:02 |
| 4391 | MAGEA4 | ENSG00000147381 | EVDPASNTYTL | 2247 | HLA-B*27:05 |
| 4392 | MAGEA4 | ENSG00000147381 | EVDPASNTYTL | 2247 | HLA-B*35:03 |
| 4393 | MAGEA4 | ENSG00000147381 | EVDPASNTYTL | 2247 | HLA-B*38:01 |
| 4394 | MAGEA4 | ENSG00000147381 | EVDPASNTYTL | 2247 | HLA-B*39:01 |
| 4395 | MAGEA4 | ENSG00000147381 | EVDPASNTYTL | 2247 | HLA-B*40:01 |
| 4396 | MAGEA4 | ENSG00000147381 | EVDPASNTYTL | 2247 | HLA-C*05:01 |
| 4397 | MAGEA4 | ENSG00000147381 | EVDPASNTYTL | 2247 | HLA-C*07:06 |
| 4398 | MAGEA4 | ENSG00000147381 | EVDPASNTY | 12 | HLA-A*01:01 |
| 4399 | MAGEA4 | ENSG00000147381 | EVDPASNTY | 12 | HLA-A*25:01 |
| 4400 | MAGEA4 | ENSG00000147381 | EVDPASNTY | 12 | HLA-A*26:01 |
| 4401 | MAGEA4 | ENSG00000147381 | EVDPASNTY | 12 | HLA-A*29:02 |
| 4402 | MAGEA4 | ENSG00000147381 | EVDPASNTY | 12 | HLA-A*30:02 |
| 4403 | MAGEA4 | ENSG00000147381 | EVDPASNTY | 12 | HLA-A*32:01 |
| 4404 | MAGEA4 | ENSG00000147381 | EVDPASNTY | 12 | HLA-A*33:03 |
| 4405 | MAGEA4 | ENSG00000147381 | EVDPASNTY | 12 | HLA-A*68:01 |
| 4406 | MAGEA4 | ENSG00000147381 | EVDPASNTY | 12 | HLA-B*15:01 |
| 4407 | MAGEA4 | ENSG00000147381 | EVDPASNTY | 12 | HLA-B*15:03 |
| 4408 | MAGEA4 | ENSG00000147381 | EVDPASNTY | 12 | HLA-B*18:01 |
| 4409 | MAGEA4 | ENSG00000147381 | EVDPASNTY | 12 | HLA-B*35:01 |
| 4410 | MAGEA4 | ENSG00000147381 | EVDPASNTY | 12 | HLA-B*38:01 |
| 4411 | MAGEA4 | ENSG00000147381 | EVDPASNTY | 12 | HLA-B*39:01 |
| 4412 | MAGEA4 | ENSG00000147381 | EVDPASNTY | 12 | HLA-B*44:03 |
| 4413 | MAGEA4 | ENSG00000147381 | EVDPASNTY | 12 | HLA-B*46:01 |
| 4414 | MAGEA4 | ENSG00000147381 | EVDPASNTY | 12 | HLA-B*55:01 |
| 4415 | MAGEA4 | ENSG00000147381 | EVDPASNTY | 12 | HLA-B*58:01 |
| 4416 | MAGEA4 | ENSG00000147381 | EVDPASNTY | 12 | HLA-C*02:02 |
| 4417 | MAGEA4 | ENSG00000147381 | EVDPASNTY | 12 | HLA-C*03:03 |
| 4418 | MAGEA4 | ENSG00000147381 | EVDPASNTY | 12 | HLA-C*05:01 |
| 4419 | MAGEA4 | ENSG00000147381 | EVDPASNTY | 12 | HLA-C*07:04 |
| 4420 | MAGEA4 | ENSG00000147381 | EVDPASNTY | 12 | HLA-C*07:06 |
| 4421 | MAGEA4 | ENSG00000147381 | EVDPASNTY | 12 | HLA-C*12:03 |
| 4422 | MAGEA4 | ENSG00000147381 | EVDPASNTY | 12 | HLA-C*16:02 |
| 4423 | MAGEA4 | ENSG00000147381 | EVDPASNTY | 12 | HLA-C*16:04 |
| 4424 | MAGEA4 | ENSG00000147381 | EVPAAESAGP | 2248 | HLA-A*26:01 |
| 4425 | MAGEA4 | ENSG00000147381 | EYRQVPGSNP | 2249 | HLA-A*33:03 |
| 4426 | MAGEA4 | ENSG00000147381 | FGIDVKEV | 2018 | HLA-B*46:01 |
| 4427 | MAGEA4 | ENSG00000147381 | FGIDVKEV | 2018 | HLA-B*49:01 |
| 4428 | MAGEA4 | ENSG00000147381 | FGIDVKEV | 2018 | HLA-B*51:01 |
| 4429 | MAGEA4 | ENSG00000147381 | FGIDVKEV | 2018 | HLA-C*02:02 |
| 4430 | MAGEA4 | ENSG00000147381 | FGIDVKEV | 2018 | HLA-C*03:04 |
| 4431 | MAGEA4 | ENSG00000147381 | FGIDVKEV | 2018 | HLA-C*12:03 |
| 4432 | MAGEA4 | ENSG00000147381 | FGIDVKEV | 2018 | HLA-C*16:02 |
| 4433 | MAGEA4 | ENSG00000147381 | FGKASESLK | 2250 | HLA-C*07:02 |
| 4434 | MAGEA4 | ENSG00000147381 | FGKASESL | 2251 | HLA-B*46:01 |
| 4435 | MAGEA4 | ENSG00000147381 | FGKASESL | 2251 | HLA-C*03:04 |
| 4436 | MAGEA4 | ENSG00000147381 | FGKASESL | 2251 | HLA-C*14:02 |
| 4437 | MAGEA4 | ENSG00000147381 | FLWGPRALAET | 199 | HLA-A*02:01 |
| 4438 | MAGEA4 | ENSG00000147381 | FLWGPRALAET | 199 | HLA-A*02:03 |
| 4439 | MAGEA4 | ENSG00000147381 | FLWGPRALAET | 199 | HLA-A*02:04 |
| 4440 | MAGEA4 | ENSG00000147381 | FLWGPRALAET | 199 | HLA-A*02:07 |
| 4441 | MAGEA4 | ENSG00000147381 | FLWGPRALA | 2252 | HLA-A*02:01 |
| 4442 | MAGEA4 | ENSG00000147381 | FLWGPRAL | 2253 | HLA-A*02:04 |
| 4443 | MAGEA4 | ENSG00000147381 | FPKTGLLII | 2254 | HLA-B*51:01 |
| 4444 | MAGEA4 | ENSG00000147381 | FPKTGLLII | 2254 | HLA-B*54:01 |
| 4445 | MAGEA4 | ENSG00000147381 | FPKTGLLI | 2255 | HLA-B*51:01 |
| 4446 | MAGEA4 | ENSG00000147381 | FPVIFGKAS | 2256 | HLA-B*54:01 |
| 4447 | MAGEA4 | ENSG00000147381 | FPVIFGKA | 2257 | HLA-B*54:01 |
| 4448 | MAGEA4 | ENSG00000147381 | FPVIFGKA | 2257 | HLA-B*55:01 |
| 4449 | MAGEA4 | ENSG00000147381 | FPVIFGKA | 2257 | HLA-B*56:01 |
| 4450 | MAGEA4 | ENSG00000147381 | FREALSNKV | 2258 | HLA-C*06:02 |
| 4451 | MAGEA4 | ENSG00000147381 | GASALPTTI | 2259 | HLA-B*13:02 |
| 4452 | MAGEA4 | ENSG00000147381 | GASALPTTI | 2259 | HLA-B*15:03 |
| 4453 | MAGEA4 | ENSG00000147381 | GASALPTTI | 2259 | HLA-B*49:01 |
| 4454 | MAGEA4 | ENSG00000147381 | GASALPTTI | 2259 | HLA-B*51:01 |
| 4455 | MAGEA4 | ENSG00000147381 | GASALPTTI | 2259 | HLA-B*58:01 |
| 4456 | MAGEA4 | ENSG00000147381 | GASALPTTI | 2259 | HLA-C*02:02 |
| 4457 | MAGEA4 | ENSG00000147381 | GASALPTTI | 2259 | HLA-C*I2:03 |
| 4458 | MAGEA4 | ENSG00000147381 | GASALPTTI | 2259 | HLA-C*16:02 |
| 4459 | MAGEA4 | ENSG00000147381 | GASALPTTI | 2259 | HLA-C*16:04 |
| 4460 | MAGEA4 | ENSG00000147381 | GKASESLKM | 2260 | HLA-B*15:03 |
| 4461 | MAGEA4 | ENSG00000147381 | GLLGNNQIFPK | 2261 | HLA-A*03:01 |
| 4462 | MAGEA4 | ENSG00000147381 | GLLGNNQIFPK | 2261 | HLA-A*03:02 |
| 4463 | MAGEA4 | ENSG00000147381 | GLLGNNQIFPK | 2261 | HLA-A*11:01 |
| 4464 | MAGEA4 | ENSG00000147381 | GLLGNNQIFPK | 2261 | HLA-A*31:01 |
| 4465 | MAGEA4 | ENSG00000147381 | GLLGNNQIF | 2262 | HLA-B*15:01 |
| 4466 | MAGEA4 | ENSG00000147381 | GLLIIVLGTI | 2263 | HLA-A*02:04 |
| 4467 | MAGEA4 | ENSG00000147381 | GPPQSPQGASA | 2264 | HLA-B*56:01 |
| 4468 | MAGEA4 | ENSG00000147381 | GPRALAETSYV | 2265 | HLA-C*07:02 |
| 4469 | MAGEA4 | ENSG00000147381 | GPRALAETSY | 2266 | HLA-A*30:02 |
| 4470 | MAGEA4 | ENSG00000147381 | GPRALAETSY | 2266 | HLA-B*07:02 |
| 4471 | MAGEA4 | ENSG00000147381 | GPRALAETSY | 2266 | HLA-B*15:01 |
| 4472 | MAGEA4 | ENSG00000147381 | GPRALAETSY | 2266 | HLA-B*15:03 |
| 4473 | MAGEA4 | ENSG00000147381 | GPRALAETSY | 2266 | HLA-B*35:01 |
| 4474 | MAGEA4 | ENSG00000147381 | GPRALAETSY | 2266 | HLA-B*55:01 |
| 4475 | MAGEA4 | ENSG00000147381 | GSNPARYEFLW | 2267 | HLA-B*57:01 |
| 4476 | MAGEA4 | ENSG00000147381 | GSNPARYEF | 2268 | HLA-A*23:01 |
| 4477 | MAGEA4 | ENSG00000147381 | GSNPARYEF | 2268 | HLA-A*29:02 |
| 4478 | MAGEA4 | ENSG00000147381 | GSNPARYEF | 2268 | HLA-A*30:02 |
| 4479 | MAGEA4 | ENSG00000147381 | GSNPARYEF | 2268 | HLA-A*31:01 |
| 4480 | MAGEA4 | ENSG00000147381 | GSNPARYEF | 2268 | HLA-A*32:01 |
| 4481 | MAGEA4 | ENSG00000147381 | GSNPARYEF | 2268 | HLA-B*15:01 |
| 4482 | MAGEA4 | ENSG00000147381 | GSNPARYEF | 2268 | HLA-B*15:03 |
| 4483 | MAGEA4 | ENSG00000147381 | GSNPARYEF | 2268 | HLA-B*46:01 |
| 4484 | MAGEA4 | ENSG00000147381 | GSNPARYEF | 2268 | HLA-B*57:01 |
| 4485 | MAGEA4 | ENSG00000147381 | GSNPARYEF | 2268 | HLA-B*58:01 |
| 4486 | MAGEA4 | ENSG00000147381 | GSNPARYEF | 2268 | HLA-C*02:02 |
| 4487 | MAGEA4 | ENSG00000147381 | GSNPARYEF | 2268 | HLA-C*16:01 |
| 4488 | MAGEA4 | ENSG00000147381 | GSNPARYEF | 2268 | HLA-C*16:04 |
| 4489 | MAGEA4 | ENSG00000147381 | GTLEEVPAA | 2269 | HLA-A*02:01 |
| 4490 | MAGEA4 | ENSG00000147381 | GVMGVYDGR | 2270 | HLA-A*03:02 |
| 4491 | MAGEA4 | ENSG00000147381 | GVMGVYDGR | 2270 | HLA-A*11:01 |
| 4492 | MAGEA4 | ENSG00000147381 | GVMGVYDGR | 2270 | HLA-A*31:01 |
| 4493 | MAGEA4 | ENSG00000147381 | GVMGVYDGR | 2270 | HLA-A*33:03 |
| 4494 | MAGEA4 | ENSG00000147381 | GVMGVYDGR | 2270 | HLA-A*68:01 |
| 4495 | MAGEA4 | ENSG00000147381 | GVMGVYDGR | 2270 | HLA-C*07:06 |
| 4496 | MAGEA4 | ENSG00000147381 | GVYDGREHTVY | 2271 | HLA-A*01:01 |
| 4497 | MAGEA4 | ENSG00000147381 | GVYDGREHTVY | 2271 | HLA-A*03:01 |
| 4498 | MAGEA4 | ENSG00000147381 | GVYDGREHTVY | 2271 | HLA-A*03:02 |
| 4499 | MAGEA4 | ENSG00000147381 | GVYDGREHTVY | 2271 | HLA-A*11:01 |
| 4500 | MAGEA4 | ENSG00000147381 | GVYDGREHTVY | 2271 | HLA-A*25:01 |
| 4501 | MAGEA4 | ENSG00000147381 | GVYDGREHTVY | 2271 | HLA-A*26:01 |
| 4502 | MAGEA4 | ENSG00000147381 | GVYDGREHTVY | 2271 | HLA-A*29:02 |
| 4503 | MAGEA4 | ENSG00000147381 | GVYDGREHTVY | 2271 | HLA-A*30:02 |
| 4504 | MAGEA4 | ENSG00000147381 | GVYDGREHTVY | 2271 | HLA-A*32:01 |
| 4505 | MAGEA4 | ENSG00000147381 | GVYDGREHTVY | 2271 | HLA-B*15:01 |
| 4506 | MAGEA4 | ENSG00000147381 | GVYDGREHTVY | 2271 | HLA-B*15:03 |
| 4507 | MAGEA4 | ENSG00000147381 | GVYDGREHTVY | 2271 | HLA-B*27:05 |
| 4508 | MAGEA4 | ENSG00000147381 | GVYDGREHTVY | 2271 | HLA-B*35:01 |
| 4509 | MAGEA4 | ENSG00000147381 | GVYDGREHTVY | 2271 | HLA-B*46:01 |
| 4510 | MAGEA4 | ENSG00000147381 | GVYDGREHTVY | 2271 | HLA-B*55:01 |
| 4511 | MAGEA4 | ENSG00000147381 | GVYDGREHTVY | 2271 | HLA-B*58:01 |
| 4512 | MAGEA4 | ENSG00000147381 | GVYDGREHTVY | 2271 | HLA-C*02:02 |
| 4513 | MAGEA4 | ENSG00000147381 | GVYDGREHTVY | 2271 | HLA-C*07:04 |
| 4514 | MAGEA4 | ENSG00000147381 | GVYDGREHTVY | 2271 | HLA-C*12:03 |
| 4515 | MAGEA4 | ENSG00000147381 | GVYDGREHTVY | 2271 | HLA-C*16:01 |
| 4516 | MAGEA4 | ENSG00000147381 | GVYDGREHTVY | 2271 | HLA-C*16:04 |
| 4517 | MAGEA4 | ENSG00000147381 | GVYDGREHTV | 2272 | HLA-A*02:01 |
| 4518 | MAGEA4 | ENSG00000147381 | GVYDGREHTV | 2272 | HLA-A*02:03 |
| 4519 | MAGEA4 | ENSG00000147381 | GVYDGREHTV | 2272 | HLA-A*02:04 |
| 4520 | MAGEA4 | ENSG00000147381 | GVYDGREHTV | 2272 | HLA-A*02:07 |
| 4521 | MAGEA4 | ENSG00000147381 | GVYDGREHTV | 2272 | HLA-A*03:01 |
| 4522 | MAGEA4 | ENSG00000147381 | GVYDGREHTV | 2272 | HLA-A*25:01 |
| 4523 | MAGEA4 | ENSG00000147381 | GVYDGREHTV | 2272 | HLA-A*26:01 |
| 4524 | MAGEA4 | ENSG00000147381 | GVYDGREHTV | 2272 | HLA-A*30:01 |
| 4525 | MAGEA4 | ENSG00000147381 | GVYDGREHTV | 2272 | HLA-A*32:01 |
| 4526 | MAGEA4 | ENSG00000147381 | GVYDGREHTV | 2272 | HLA-A*68:02 |
| 4527 | MAGEA4 | ENSG00000147381 | GVYDGREHTV | 2272 | HLA-B*13:02 |
| 4528 | MAGEA4 | ENSG00000147381 | GVYDGREHTV | 2272 | HLA-B*15:01 |
| 4529 | MAGEA4 | ENSG00000147381 | GVYDGREHTV | 2272 | HLA-B*27:05 |
| 4530 | MAGEA4 | ENSG00000147381 | GVYDGREHTV | 2272 | HLA-B*37:01 |
| 4531 | MAGEA4 | ENSG00000147381 | GVYDGREHTV | 2272 | HLA-B*40:02 |
| 4532 | MAGEA4 | ENSG00000147381 | GVYDGREHTV | 2272 | HLA-B*46:01 |
| 4533 | MAGEA4 | ENSG00000147381 | GVYDGREHTV | 2272 | HLA-B*49:01 |
| 4534 | MAGEA4 | ENSG00000147381 | GVYDGREHTV | 2272 | HLA-B*51:01 |
| 4535 | MAGEA4 | ENSG00000147381 | GVYDGREHTV | 2272 | HLA-B*55:01 |
| 4536 | MAGEA4 | ENSG00000147381 | GVYDGREHTV | 2272 | HLA-B*56:01 |
| 4537 | MAGEA4 | ENSG00000147381 | GVYDGREHTV | 2272 | HLA-B*58:01 |
| 4538 | MAGEA4 | ENSG00000147381 | GVYDGREHTV | 2272 | HLA-C*01:02 |
| 4539 | MAGEA4 | ENSG00000147381 | GVYDGREHTV | 2272 | HLA-C*02:02 |
| 4540 | MAGEA4 | ENSG00000147381 | GVYDGREHTV | 2272 | HLA-C*03:03 |
| 4541 | MAGEA4 | ENSG00000147381 | GVYDGREHTV | 2272 | HLA-C*03:04 |
| 4542 | MAGEA4 | ENSG00000147381 | GVYDGREHTV | 2272 | HLA-C*06:02 |
| 4543 | MAGEA4 | ENSG00000147381 | GVYDGREHTV | 2272 | HLA-C*07:02 |
| 4544 | MAGEA4 | ENSG00000147381 | GVYDGREHTV | 2272 | HLA-C*12:03 |
| 4545 | MAGEA4 | ENSG00000147381 | GVYDGREHTV | 2272 | HLA-C*16:02 |
| 4546 | MAGEA4 | ENSG00000147381 | GVYDGREHTV | 2272 | HLA-C*16:04 |
| 4547 | MAGEA4 | ENSG00000147381 | HTVYGEPR | 2273 | HLA-A*68:01 |
| 4548 | MAGEA4 | ENSG00000147381 | HVVRVNARV | 2274 | HLA-A*68:02 |
| 4549 | MAGEA4 | ENSG00000147381 | IAMEGDSA | 2275 | HLA-B*54:01 |
| 4550 | MAGEA4 | ENSG00000147381 | IAMEGDSA | 2275 | HLA-C*01:02 |
| 4551 | MAGEA4 | ENSG00000147381 | IAMEGDSA | 2275 | HLA-C*03:03 |
| 4552 | MAGEA4 | ENSG00000147381 | IAMEGDSA | 2275 | HLA-C*03:04 |
| 4553 | MAGEA4 | ENSG00000147381 | IAMEGDSA | 2275 | HLA-C*05:01 |
| 4554 | MAGEA4 | ENSG00000147381 | IAMEGDSA | 2275 | HLA-C*14:02 |
| 4555 | MAGEA4 | ENSG00000147381 | IAYPSLREAAL | 2276 | HLA-B*07:02 |
| 4556 | MAGEA4 | ENSG00000147381 | IAYPSLREAAL | 2276 | HLA-B*35:03 |
| 4557 | MAGEA4 | ENSG00000147381 | IAYPSLREAAL | 2276 | HLA-B*46:01 |
| 4558 | MAGEA4 | ENSG00000147381 | IAYPSLREAAL | 2276 | HLA-C*01:02 |
| 4559 | MAGEA4 | ENSG00000147381 | IAYPSLREAAL | 2276 | HLA-C*02:02 |
| 4560 | MAGEA4 | ENSG00000147381 | IAYPSLREAAL | 2276 | HLA-C*03:03 |
| 4561 | MAGEA4 | ENSG00000147381 | IAYPSLREAAL | 2276 | HLA-C*03:04 |
| 4562 | MAGEA4 | ENSG00000147381 | IAYPSLREAAL | 2276 | HLA-C*16:01 |
| 4563 | MAGEA4 | ENSG00000147381 | IAYPSLREAAL | 2276 | HLA-C*16:02 |
| 4564 | MAGEA4 | ENSG00000147381 | IAYPSLREAA | 2277 | HLA-B*54:01 |
| 4565 | MAGEA4 | ENSG00000147381 | IAYPSLREAA | 2277 | HLA-B*56:01 |
| 4566 | MAGEA4 | ENSG00000147381 | IAYPSLREA | 2278 | HLA-A*02:01 |
| 4567 | MAGEA4 | ENSG00000147381 | IAYPSLREA | 2278 | HLA-A*02:03 |
| 4568 | MAGEA4 | ENSG00000147381 | IAYPSLREA | 2278 | HLA-B*08:01 |
| 4569 | MAGEA4 | ENSG00000147381 | IAYPSLREA | 2278 | HLA-B*46:01 |
| 4570 | MAGEA4 | ENSG00000147381 | IAYPSLREA | 2278 | HLA-B*51:01 |
| 4571 | MAGEA4 | ENSG00000147381 | IAYPSLREA | 2278 | HLA-B*54:01 |
| 4572 | MAGEA4 | ENSG00000147381 | IAYPSLREA | 2278 | HLA-B*55:01 |
| 4573 | MAGEA4 | ENSG00000147381 | IAYPSLREA | 2278 | HLA-B*56:01 |
| 4574 | MAGEA4 | ENSG00000147381 | IAYPSLREA | 2278 | HLA-B*57:01 |
| 4575 | MAGEA4 | ENSG00000147381 | IAYPSLREA | 2278 | HLA-B*58:01 |
| 4576 | MAGEA4 | ENSG00000147381 | IAYPSLREA | 2278 | HLA-C*02:02 |
| 4577 | MAGEA4 | ENSG00000147381 | IAYPSLREA | 2278 | HLA-C*03:03 |
| 4578 | MAGEA4 | ENSG00000147381 | IAYPSLREA | 2278 | HLA-C*03:04 |
| 4579 | MAGEA4 | ENSG00000147381 | IAYPSLREA | 2278 | HLA-C*06:02 |
| 4580 | MAGEA4 | ENSG00000147381 | IAYPSLREA | 2278 | HLA-C*12:03 |
| 4581 | MAGEA4 | ENSG00000147381 | IAYPSLREA | 2278 | HLA-C*16:01 |
| 4582 | MAGEA4 | ENSG00000147381 | IAYPSLREA | 2278 | HLA-C*16:02 |
| 4583 | MAGEA4 | ENSG00000147381 | IFGKASESL | 2279 | HLA-C*01:02 |
| 4584 | MAGEA4 | ENSG00000147381 | IFGKASESL | 2279 | HLA-C*14:02 |
| 4585 | MAGEA4 | ENSG00000147381 | IFPKTGLLII | 2280 | HLA-A*24:02 |
| 4586 | MAGEA4 | ENSG00000147381 | IFPKTGLLI | 2281 | HLA-A*23:01 |
| 4587 | MAGEA4 | ENSG00000147381 | IFPKTGLLI | 2281 | HLA-A*24:02 |
| 4588 | MAGEA4 | ENSG00000147381 | IIVLGTIAM | 2282 | HLA-B*08:01 |
| 4589 | MAGEA4 | ENSG00000147381 | IIVLGTIAM | 2282 | HLA-B*15:01 |
| 4590 | MAGEA4 | ENSG00000147381 | IIVLGTIAM | 2282 | HLA-B*35:01 |
| 4591 | MAGEA4 | ENSG00000147381 | IIVLGTIAM | 2282 | HLA-B*35:03 |
| 4592 | MAGEA4 | ENSG00000147381 | IIVLGTIAM | 2282 | HLA-B*39:01 |
| 4593 | MAGEA4 | ENSG00000147381 | IIVLGTIAM | 2282 | HLA-B*46:01 |
| 4594 | MAGEA4 | ENSG00000147381 | IIVLGTIAM | 2282 | HLA-C*01:02 |
| 4595 | MAGEA4 | ENSG00000147381 | IIVLGTIAM | 2282 | HLA-C*03:03 |
| 4596 | MAGEA4 | ENSG00000147381 | IIVLGTIAM | 2282 | HLA-C*03:04 |
| 4597 | MAGEA4 | ENSG00000147381 | IIVLGTIAM | 2282 | HLA-C*07:04 |
| 4598 | MAGEA4 | ENSG00000147381 | IIVLGTIAM | 2282 | HLA-C*14:02 |
| 4599 | MAGEA4 | ENSG00000147381 | IVLGTIAMEG | 2283 | HLA-C*04:01 |
| 4600 | MAGEA4 | ENSG00000147381 | IVLGTIAM | 2284 | HLA-A*23:01 |
| 4601 | MAGEA4 | ENSG00000147381 | IVLGTIAM | 2284 | HLA-B*46:01 |
| 4602 | MAGEA4 | ENSG00000147381 | IVLGTIAM | 2284 | HLA-C*01:02 |
| 4603 | MAGEA4 | ENSG00000147381 | IVLGTIAM | 2284 | HLA-C*03:03 |
| 4604 | MAGEA4 | ENSG00000147381 | IVLGTIAM | 2284 | HLA-C*14:02 |
| 4605 | MAGEA4 | ENSG00000147381 | KAEMLERVI | 2285 | HLA-C*16:02 |
| 4606 | MAGEA4 | ENSG00000147381 | KASESLKMIF | 2286 | HLA-B*57:01 |
| 4607 | MAGEA4 | ENSG00000147381 | KASESLKMI | 2287 | HLA-C*16:02 |
| 4608 | MAGEA4 | ENSG00000147381 | KASESLKM | 2288 | HLA-B*37:01 |
| 4609 | MAGEA4 | ENSG00000147381 | KASESLKM | 2288 | HLA-B*58:01 |
| 4610 | MAGEA4 | ENSG00000147381 | KASESLKM | 2288 | HLA-C*03:04 |
| 4611 | MAGEA4 | ENSG00000147381 | KASESLKM | 2288 | HLA-C*16:01 |
| 4612 | MAGEA4 | ENSG00000147381 | KASESLKM | 2288 | HLA-C*16:02 |
| 4613 | MAGEA4 | ENSG00000147381 | KELVTKAEML | 2289 | HLA-A*30:01 |
| 4614 | MAGEA4 | ENSG00000147381 | KELVTKAEML | 2289 | HLA-B*40:01 |
| 4615 | MAGEA4 | ENSG00000147381 | KELVTKAEML | 2289 | HLA-B*40:02 |
| 4616 | MAGEA4 | ENSG00000147381 | KELVTKAEM | 2290 | HLA-A*30:01 |
| 4617 | MAGEA4 | ENSG00000147381 | KELVTKAEM | 2290 | HLA-B*18:01 |
| 4618 | MAGEA4 | ENSG00000147381 | KELVTKAEM | 2290 | HLA-B*37:01 |
| 4619 | MAGEA4 | ENSG00000147381 | KELVTKAEM | 2290 | HLA-B*40:01 |
| 4620 | MAGEA4 | ENSG00000147381 | KELVTKAEM | 2290 | HLA-B*40:02 |
| 4621 | MAGEA4 | ENSG00000147381 | KELVTKAEM | 2290 | HLA-B*44:02 |
| 4622 | MAGEA4 | ENSG00000147381 | KELVTKAEM | 2290 | HLA-B*44:03 |
| 4623 | MAGEA4 | ENSG00000147381 | KELVTKAEM | 2290 | HLA-B*49:01 |
| 4624 | MAGEA4 | ENSG00000147381 | KEVDPASNTYT | 2291 | HLA-B*40:01 |
| 4625 | MAGEA4 | ENSG00000147381 | KEVDPASNTYT | 2291 | HLA-B*49:01 |
| 4626 | MAGEA4 | ENSG00000147381 | KEVDPASNTY | 19 | HLA-A*25:01 |
| 4627 | MAGEA4 | ENSG00000147381 | KEVDPASNTY | 19 | HLA-A*26:01 |
| 4628 | MAGEA4 | ENSG00000147381 | KEVDPASNTY | 19 | HLA-A*29:02 |
| 4629 | MAGEA4 | ENSG00000147381 | KEVDPASNTY | 19 | HLA-A*30:01 |
| 4630 | MAGEA4 | ENSG00000147381 | KEVDPASNTY | 19 | HLA-A*30:02 |
| 4631 | MAGEA4 | ENSG00000147381 | KEVDPASNTY | 19 | HLA-A*32:01 |
| 4632 | MAGEA4 | ENSG00000147381 | KEVDPASNTY | 19 | HLA-B*15:01 |
| 4633 | MAGEA4 | ENSG00000147381 | KEVDPASNTY | 19 | HLA-B*15:03 |
| 4634 | MAGEA4 | ENSG00000147381 | KEVDPASNTY | 19 | HLA-B*18:01 |
| 4635 | MAGEA4 | ENSG00000147381 | KEVDPASNTY | 19 | HLA-B*27:02 |
| 4636 | MAGEA4 | ENSG00000147381 | KEVDPASNTY | 19 | HLA-B*27:05 |
| 4637 | MAGEA4 | ENSG00000147381 | KEVDPASNTY | 19 | HLA-B*37:01 |
| 4638 | MAGEA4 | ENSG00000147381 | KEVDPASNTY | 19 | HLA-B*39:01 |
| 4639 | MAGEA4 | ENSG00000147381 | KEVDPASNTY | 19 | HLA-B*40:01 |
| 4640 | MAGEA4 | ENSG00000147381 | KEVDPASNTY | 19 | HLA-B*40:02 |
| 4641 | MAGEA4 | ENSG00000147381 | KEVDPASNTY | 19 | HLA-B*44:02 |
| 4642 | MAGEA4 | ENSG00000147381 | KEVDPASNTY | 19 | HLA-B*44:03 |
| 4643 | MAGEA4 | ENSG00000147381 | KEVDPASNTY | 19 | HLA-B*46:01 |
| 4644 | MAGEA4 | ENSG00000147381 | KEVDPASNTY | 19 | HLA-B*49:01 |
| 4645 | MAGEA4 | ENSG00000147381 | KEVDPASNTY | 19 | HLA-B*57:01 |
| 4646 | MAGEA4 | ENSG00000147381 | KEVDPASNTY | 19 | HLA-B*58:01 |
| 4647 | MAGEA4 | ENSG00000147381 | KEVDPASNTY | 19 | HLA-C*02:02 |
| 4648 | MAGEA4 | ENSG00000147381 | KEVDPASNTY | 19 | HLA-C*12:03 |
| 4649 | MAGEA4 | ENSG00000147381 | KEVDPASNTY | 19 | HLA-C*14:02 |
| 4650 | MAGEA4 | ENSG00000147381 | KEVDPASNTY | 19 | HLA-C*16:01 |
| 4651 | MAGEA4 | ENSG00000147381 | KEVDPASNTY | 19 | HLA-C*16:02 |
| 4652 | MAGEA4 | ENSG00000147381 | KEVDPASNTY | 19 | HLA-C*16:04 |
| 4653 | MAGEA4 | ENSG00000147381 | KEVDPASNT | 2292 | HLA-B*40:01 |
| 4654 | MAGEA4 | ENSG00000147381 | KEVDPASNT | 2292 | HLA-B*49:01 |
| 4655 | MAGEA4 | ENSG00000147381 | KMIFGIDVKEV | 2293 | HLA-A*02:01 |
| 4656 | MAGEA4 | ENSG00000147381 | KVDELAHFLLR | 2294 | HLA-A*02:07 |
| 4657 | MAGEA4 | ENSG00000147381 | KVDELAHFLLR | 2294 | HLA-A*03:01 |
| 4658 | MAGEA4 | ENSG00000147381 | KVDELAHFLLR | 2294 | HLA-A*31:01 |
| 4659 | MAGEA4 | ENSG00000147381 | KVDELAHFLL | 16 | HLA-A*02:01 |
| 4660 | MAGEA4 | ENSG00000147381 | KVDELAHFLL | 16 | HLA-A*02:04 |
| 4661 | MAGEA4 | ENSG00000147381 | KVDELAHFLL | 16 | HLA-A*02:07 |
| 4662 | MAGEA4 | ENSG00000147381 | KVDELAHFLL | 16 | HLA-A*31:01 |
| 4663 | MAGEA4 | ENSG00000147381 | KVDELAHFL | 13 | HLA-A*02:01 |
| 4664 | MAGEA4 | ENSG00000147381 | KVDELAHFL | 13 | HLA-A*02:04 |
| 4665 | MAGEA4 | ENSG00000147381 | KVDELAHFL | 13 | HLA-A*02:07 |
| 4666 | MAGEA4 | ENSG00000147381 | KVDELAHFL | 13 | HLA-A*03:02 |
| 4667 | MAGEA4 | ENSG00000147381 | KVDELAHFL | 13 | HLA-A*31:01 |
| 4668 | MAGEA4 | ENSG00000147381 | KVDELAHFL | 13 | HLA-A*68:02 |
| 4669 | MAGEA4 | ENSG00000147381 | KVDELAHFL | 13 | HLA-B*13:02 |
| 4670 | MAGEA4 | ENSG00000147381 | KVDELAHFL | 13 | HLA-B*38:01 |
| 4671 | MAGEA4 | ENSG00000147381 | KVDELAHFL | 13 | HLA-B*58:01 |
| 4672 | MAGEA4 | ENSG00000147381 | KVDELAHFL | 13 | HLA-C*05:01 |
| 4673 | MAGEA4 | ENSG00000147381 | KVDELAHF | 2295 | HLA-C*05:01 |
| 4674 | MAGEA4 | ENSG00000147381 | KVLEHVVRVNA | 2296 | HLA-A*31:01 |
| 4675 | MAGEA4 | ENSG00000147381 | KVLEHVVRV | 2297 | HLA-A*02:01 |
| 4676 | MAGEA4 | ENSG00000147381 | KVLEHVVRV | 2297 | HLA-A*02:03 |
| 4677 | MAGEA4 | ENSG00000147381 | KVLEHVVRV | 2297 | HLA-A*02:04 |
| 4678 | MAGEA4 | ENSG00000147381 | KVLEHVVRV | 2297 | HLA-A*02:07 |
| 4679 | MAGEA4 | ENSG00000147381 | KVLEHVVRV | 2297 | HLA-A*03:01 |
| 4680 | MAGEA4 | ENSG00000147381 | KVLEHVVRV | 2297 | HLA-A*03:02 |
| 4681 | MAGEA4 | ENSG00000147381 | KVLEHVVRV | 2297 | HLA-A*31:01 |
| 4682 | MAGEA4 | ENSG00000147381 | KVLEHVVRV | 2297 | HLA-A*68:02 |
| 4683 | MAGEA4 | ENSG00000147381 | KVLEHVVRV | 2297 | HLA-B*13:02 |
| 4684 | MAGEA4 | ENSG00000147381 | KVLEHVVRV | 2297 | HLA-B*37:01 |
| 4685 | MAGEA4 | ENSG00000147381 | KVLEHVVRV | 2297 | HLA-B*55:01 |
| 4686 | MAGEA4 | ENSG00000147381 | KVLEHVVRV | 2297 | HLA-C*02:02 |
| 4687 | MAGEA4 | ENSG00000147381 | KVLEHVVR | 2298 | HLA-A*31:01 |
| 4688 | MAGEA4 | ENSG00000147381 | LAETSYVKV | 2299 | HLA-C*05:01 |
| 4689 | MAGEA4 | ENSG00000147381 | LAETSYVK | 2300 | HLA-B*27:02 |
| 4690 | MAGEA4 | ENSG00000147381 | LAHFLLRKY | 2301 | HLA-A*29:02 |
| 4691 | MAGEA4 | ENSG00000147381 | LEHVVRVNA | 2302 | HLA-B*40:02 |
| 4692 | MAGEA4 | ENSG00000147381 | LERVIKNY | 2303 | HLA-B*18:01 |
| 4693 | MAGEA4 | ENSG00000147381 | LGNNQIFPK | 2304 | HLA-A*03:02 |
| 4694 | MAGEA4 | ENSG00000147381 | LGNNQIFPK | 2304 | HLA-A*11:01 |
| 4695 | MAGEA4 | ENSG00000147381 | LGNNQIFPK | 2304 | HLA-B*27:02 |
| 4696 | MAGEA4 | ENSG00000147381 | LGNNQIFPK | 2304 | HLA-C*07:06 |
| 4697 | MAGEA4 | ENSG00000147381 | LGVMGVYDGR | 2305 | HLA-B*27:02 |
| 4698 | MAGEA4 | ENSG00000147381 | LIIVLGTIAM | 2306 | HLA-B*46:01 |
| 4699 | MAGEA4 | ENSG00000147381 | LLGNNQIFPK | 2307 | HLA-A*03:01 |
| 4700 | MAGEA4 | ENSG00000147381 | LLGNNQIFPK | 2307 | HLA-A*03:02 |
| 4701 | MAGEA4 | ENSG00000147381 | LLGNNQIFPK | 2307 | HLA-B*27:02 |
| 4702 | MAGEA4 | ENSG00000147381 | LLIIVLGTI | 2308 | HLA-A*02:03 |
| 4703 | MAGEA4 | ENSG00000147381 | LPTTISFTCW | 2309 | HLA-B*35:01 |
| 4704 | MAGEA4 | ENSG00000147381 | LPTTISFTCW | 2309 | HLA-B*51:01 |
| 4705 | MAGEA4 | ENSG00000147381 | LPTTISFTCW | 2309 | HLA-B*54:01 |
| 4706 | MAGEA4 | ENSG00000147381 | LPTTISFTC | 2310 | HLA-B*35:03 |
| 4707 | MAGEA4 | ENSG00000147381 | LPTTISFTC | 2310 | HLA-B*54:01 |
| 4708 | MAGEA4 | ENSG00000147381 | LPTTISFTC | 2310 | HLA-B*56:01 |
| 4709 | MAGEA4 | ENSG00000147381 | LSNKVDELAHF | 2311 | HLA-B*57:01 |
| 4710 | MAGEA4 | ENSG00000147381 | LSYDGLLGNN | 2312 | HLA-C*06:02 |
| 4711 | MAGEA4 | ENSG00000147381 | LSYDGLLGNN | 2312 | HLA-C*12:03 |
| 4712 | MAGEA4 | ENSG00000147381 | LTQDWVQENYL | 2079 | HLA-C*04:01 |
| 4713 | MAGEA4 | ENSG00000147381 | LTQDWVQENY | 2080 | HLA-A*01:01 |
| 4714 | MAGEA4 | ENSG00000147381 | LTQDWVQENY | 2080 | HLA-A*29:02 |
| 4715 | MAGEA4 | ENSG00000147381 | LTQDWVQENY | 2080 | HLA-A*30:02 |
| 4716 | MAGEA4 | ENSG00000147381 | LTQDWVQENY | 2080 | HLA-A*32:01 |
| 4717 | MAGEA4 | ENSG00000147381 | LTQDWVQENY | 2080 | HLA-B*57:01 |
| 4718 | MAGEA4 | ENSG00000147381 | LTQDWVQENY | 2080 | HLA-B*58:01 |
| 4719 | MAGEA4 | ENSG00000147381 | LTQDWVQENY | 2080 | HLA-C*07:01 |
| 4720 | MAGEA4 | ENSG00000147381 | LVPGTLEEV | 2313 | HLA-A*02:01 |
| 4721 | MAGEA4 | ENSG00000147381 | LVPGTLEEV | 2313 | HLA-A*02:07 |
| 4722 | MAGEA4 | ENSG00000147381 | LVTCLGLSY | 2314 | HLA-A*01:01 |
| 4723 | MAGEA4 | ENSG00000147381 | LVTCLGLSY | 2314 | HLA-A*26:01 |
| 4724 | MAGEA4 | ENSG00000147381 | LVTCLGLSY | 2314 | HLA-A*29:02 |
| 4725 | MAGEA4 | ENSG00000147381 | LVTCLGLSY | 2314 | HLA-A*30:02 |
| 4726 | MAGEA4 | ENSG00000147381 | MIFGIDVKEV | 2315 | HLA-A*02:01 |
| 4727 | MAGEA4 | ENSG00000147381 | MIFGIDVKEV | 2315 | HLA-A*02:03 |
| 4728 | MAGEA4 | ENSG00000147381 | MIFGIDVKEV | 2315 | HLA-A*02:04 |
| 4729 | MAGEA4 | ENSG00000147381 | MIFGIDVKEV | 2315 | HLA-A*02:07 |
| 4730 | MAGEA4 | ENSG00000147381 | MIFGIDVKEV | 2315 | HLA-A*68:02 |
| 4731 | MAGEA4 | ENSG00000147381 | MLERVIKNY | 2316 | HLA-A*01:01 |
| 4732 | MAGEA4 | ENSG00000147381 | MLERVIKNY | 2316 | HLA-A*29:02 |
| 4733 | MAGEA4 | ENSG00000147381 | MLERVIKNY | 2316 | HLA-A*30:02 |
| 4734 | MAGEA4 | ENSG00000147381 | MLERVIKNY | 2316 | HLA-B*44:02 |
| 4735 | MAGEA4 | ENSG00000147381 | NARVRIAY | 2317 | HLA-C*16:01 |
| 4736 | MAGEA4 | ENSG00000147381 | NKVDELAHF | 2318 | HLA-A*23:01 |
| 4737 | MAGEA4 | ENSG00000147381 | NKVDELAHF | 2318 | HLA-B*15:03 |
| 4738 | MAGEA4 | ENSG00000147381 | NQIFPKTGL | 2319 | HLA-B*13:02 |
| 4739 | MAGEA4 | ENSG00000147381 | NQIFPKTGL | 2319 | HLA-B*15:01 |
| 4740 | MAGEA4 | ENSG00000147381 | NQIFPKTGL | 2319 | HLA-B*15:03 |
| 4741 | MAGEA4 | ENSG00000147381 | NQIFPKTGL | 2319 | HLA-B*27:05 |
| 4742 | MAGEA4 | ENSG00000147381 | NQIFPKTGL | 2319 | HLA-B*38:01 |
| 4743 | MAGEA4 | ENSG00000147381 | NQIFPKTGL | 2319 | HLA-B*39:01 |
| 4744 | MAGEA4 | ENSG00000147381 | NTYTLVTCL | 2320 | HLA-A*23:01 |
| 4745 | MAGEA4 | ENSG00000147381 | NTYTLVTCL | 2320 | HLA-A*25:01 |
| 4746 | MAGEA4 | ENSG00000147381 | NTYTLVTCL | 2320 | HLA-A*26:01 |
| 4747 | MAGEA4 | ENSG00000147381 | NTYTLVTCL | 2320 | HLA-A*68:01 |
| 4748 | MAGEA4 | ENSG00000147381 | NTYTLVTCL | 2320 | HLA-A*68:02 |
| 4749 | MAGEA4 | ENSG00000147381 | NTYTLVTCL | 2320 | HLA-B*18:01 |
| 4750 | MAGEA4 | ENSG00000147381 | NTYTLVTCL | 2320 | HLA-B*39:01 |
| 4751 | MAGEA4 | ENSG00000147381 | NTYTLVTCL | 2320 | HLA-C*06:02 |
| 4752 | MAGEA4 | ENSG00000147381 | NTYTLVTCL | 2320 | HLA-C*07:06 |
| 4753 | MAGEA4 | ENSG00000147381 | NTYTLVTCL | 2320 | HLA-C*12:03 |
| 4754 | MAGEA4 | ENSG00000147381 | NYKRCFPVI | 2321 | HLA-A*24:02 |
| 4755 | MAGEA4 | ENSG00000147381 | PASNTYTL | 2322 | HLA-B*07:02 |
| 4756 | MAGEA4 | ENSG00000147381 | PASNTYTL | 2322 | HLA-C*07:02 |
| 4757 | MAGEA4 | ENSG00000147381 | PDAESLFR | 2323 | HLA-B*27:02 |
| 4758 | MAGEA4 | ENSG00000147381 | PLVPGTLEEV | 2324 | HLA-A*02:01 |
| 4759 | MAGEA4 | ENSG00000147381 | PLVPGTLEEV | 2324 | HLA-A*02:03 |
| 4760 | MAGEA4 | ENSG00000147381 | PRALAETSY | 2325 | HLA-B*15:03 |
| 4761 | MAGEA4 | ENSG00000147381 | PRALAETSY | 2325 | HLA-B*27:02 |
| 4762 | MAGEA4 | ENSG00000147381 | PRALAETSY | 2325 | HLA-C*07:01 |
| 4763 | MAGEA4 | ENSG00000147381 | PRALAETSY | 2325 | HLA-C*07:04 |
| 4764 | MAGEA4 | ENSG00000147381 | PTTISFTCW | 2326 | HLA-B*57:01 |
| 4765 | MAGEA4 | ENSG00000147381 | QDWVQENYL | 2106 | HLA-A*30:01 |
| 4766 | MAGEA4 | ENSG00000147381 | QDWVQENYL | 2106 | HLA-B*37:01 |
| 4767 | MAGEA4 | ENSG00000147381 | QDWVQENY | 2327 | HLA-B*18:01 |
| 4768 | MAGEA4 | ENSG00000147381 | QEAAVSSSSPL | 2328 | HLA-A*30:01 |
| 4769 | MAGEA4 | ENSG00000147381 | QEAAVSSSSPL | 2328 | HLA-B*40:01 |
| 4770 | MAGEA4 | ENSG00000147381 | QEEALGLVGA | 2329 | HLA-A*30:01 |
| 4771 | MAGEA4 | ENSG00000147381 | QEEALGLVGA | 2329 | HLA-B*40:02 |
| 4772 | MAGEA4 | ENSG00000147381 | QEEALGLVGA | 2329 | HLA-B*49:01 |
| 4773 | MAGEA4 | ENSG00000147381 | QEEALGLV | 2330 | HLA-B*49:01 |
| 4774 | MAGEA4 | ENSG00000147381 | QIFPKTGLLII | 2331 | HLA-A*02:01 |
| 4775 | MAGEA4 | ENSG00000147381 | QIFPKTGLLII | 2331 | HLA-A*02:03 |
| 4776 | MAGEA4 | ENSG00000147381 | QIFPKTGLLII | 2331 | HLA-A*02:04 |
| 4777 | MAGEA4 | ENSG00000147381 | QIFPKTGLL | 2332 | HLA-A*02:03 |
| 4778 | MAGEA4 | ENSG00000147381 | QIFPKTGLL | 2332 | HLA-A*03:01 |
| 4779 | MAGEA4 | ENSG00000147381 | QIFPKTGL | 2333 | HLA-B*08:01 |
| 4780 | MAGEA4 | ENSG00000147381 | QSPQGASAL | 127 | HLA-C*01:02 |
| 4781 | MAGEA4 | ENSG00000147381 | QVPGSNPARY | 2334 | HLA-A*01:01 |
| 4782 | MAGEA4 | ENSG00000147381 | QVPGSNPARY | 2334 | HLA-A*25:01 |
| 4783 | MAGEA4 | ENSG00000147381 | QVPGSNPARY | 2334 | HLA-A*26:01 |
| 4784 | MAGEA4 | ENSG00000147381 | QVPGSNPARY | 2334 | HLA-A*29:02 |
| 4785 | MAGEA4 | ENSG00000147381 | QVPGSNPARY | 2334 | HLA-A*30:02 |
| 4786 | MAGEA4 | ENSG00000147381 | QVPGSNPARY | 2334 | HLA-A*32:01 |
| 4787 | MAGEA4 | ENSG00000147381 | QVPGSNPARY | 2334 | HLA-A*68:01 |
| 4788 | MAGEA4 | ENSG00000147381 | QVPGSNPARY | 2334 | HLA-C*07:04 |
| 4789 | MAGEA4 | ENSG00000147381 | QVPGSNPAR | 2335 | HLA-A*68:01 |
| 4790 | MAGEA4 | ENSG00000147381 | RAKELVTKA | 2336 | HLA-B*54:01 |
| 4791 | MAGEA4 | ENSG00000147381 | RAKELVTKA | 2336 | HLA-B*55:01 |
| 4792 | MAGEA4 | ENSG00000147381 | RALAETSYVK | 2337 | HLA-A*03:02 |
| 4793 | MAGEA4 | ENSG00000147381 | RALAETSYVK | 2337 | HLA-B*27:02 |
| 4794 | MAGEA4 | ENSG00000147381 | RCFPVIFGK | 2338 | HLA-A*03:01 |
| 4795 | MAGEA4 | ENSG00000147381 | RCFPVIFGK | 2338 | HLA-A*03:02 |
| 4796 | MAGEA4 | ENSG00000147381 | RCFPVIFGK | 2338 | HLA-A*11:01 |
| 4797 | MAGEA4 | ENSG00000147381 | RCFPVIFGK | 2338 | HLA-A*31:01 |
| 4798 | MAGEA4 | ENSG00000147381 | RCFPVIFGK | 2338 | HLA-B*57:01 |
| 4799 | MAGEA4 | ENSG00000147381 | REALSNKVDEL | 2339 | HLA-A*30:01 |
| 4800 | MAGEA4 | ENSG00000147381 | REALSNKVDEL | 2339 | HLA-B*40:01 |
| 4801 | MAGEA4 | ENSG00000147381 | REALSNKV | 2340 | HLA-B*37:01 |
| 4802 | MAGEA4 | ENSG00000147381 | REALSNKV | 2340 | HLA-B*49:01 |
| 4803 | MAGEA4 | ENSG00000147381 | RIAYPSLREA | 2341 | HLA-A*02:03 |
| 4804 | MAGEA4 | ENSG00000147381 | RQVPGSNPARY | 14 | HLA-A*29:02 |
| 4805 | MAGEA4 | ENSG00000147381 | RQVPGSNPARY | 14 | HLA-A*30:02 |
| 4806 | MAGEA4 | ENSG00000147381 | RQVPGSNPARY | 14 | HLA-A*32:01 |
| 4807 | MAGEA4 | ENSG00000147381 | RQVPGSNPARY | 14 | HLA-B*15:01 |
| 4808 | MAGEA4 | ENSG00000147381 | RQVPGSNPARY | 14 | HLA-B*15:03 |
| 4809 | MAGEA4 | ENSG00000147381 | RQVPGSNPARY | 14 | HLA-B*44:03 |
| 4810 | MAGEA4 | ENSG00000147381 | RQVPGSNPARY | 14 | HLA-B*57:01 |
| 4811 | MAGEA4 | ENSG00000147381 | RQVPGSNPARY | 14 | HLA-B*58:01 |
| 4812 | MAGEA4 | ENSG00000147381 | RQVPGSNPARY | 14 | HLA-C*02:02 |
| 4813 | MAGEA4 | ENSG00000147381 | RQVPGSNPARY | 14 | HLA-C*07:04 |
| 4814 | MAGEA4 | ENSG00000147381 | RQVPGSNPARY | 14 | HLA-C*16:02 |
| 4815 | MAGEA4 | ENSG00000147381 | RQVPGSNPARY | 14 | HLA-C*16:04 |
| 4816 | MAGEA4 | ENSG00000147381 | RQVPGSNPAR | 2342 | HLA-A*31:01 |
| 4817 | MAGEA4 | ENSG00000147381 | RV1KNYKR | 2343 | HLA-A*31:01 |
| 4818 | MAGEA4 | ENSG00000147381 | RVNARVRIAY | 2344 | HLA-A*32:01 |
| 4819 | MAGEA4 | ENSG00000147381 | RVRIAYPSLR | 2345 | HLA-A*03:01 |
| 4820 | MAGEA4 | ENSG00000147381 | RVRIAYPSLR | 2345 | HLA-A*31:01 |
| 4821 | MAGEA4 | ENSG00000147381 | RVRIAYPSL | 2346 | HLA-B*07:02 |
| 4822 | MAGEA4 | ENSG00000147381 | SAGPPQSPQGA | 2347 | HLA-C*12:03 |
| 4823 | MAGEA4 | ENSG00000147381 | SAGPPQSPQ | 2348 | HLA-C*03:03 |
| 4824 | MAGEA4 | ENSG00000147381 | SALPTTISF | 15 | HLA-A*02:01 |
| 4825 | MAGEA4 | ENSG00000147381 | SALPTTISF | 15 | HLA-A*02:04 |
| 4826 | MAGEA4 | ENSG00000147381 | SALPTTISF | 15 | HLA-A*02:07 |
| 4827 | MAGEA4 | ENSG00000147381 | SALPTTISF | 15 | HLA-A*11:01 |
| 4828 | MAGEA4 | ENSG00000147381 | SALPTTISF | 15 | HLA-A*23:01 |
| 4829 | MAGEA4 | ENSG00000147381 | SALPTTISF | 15 | HLA-A*24:02 |
| 4830 | MAGEA4 | ENSG00000147381 | SALPTTISF | 15 | HLA-A*25:01 |
| 4831 | MAGEA4 | ENSG00000147381 | SALPTTISF | 15 | HLA-A*26:01 |
| 4832 | MAGEA4 | ENSG00000147381 | SALPTTISF | 15 | HLA-A*29:02 |
| 4833 | MAGEA4 | ENSG00000147381 | SALPTTISF | 15 | HLA-A*30:01 |
| 4834 | MAGEA4 | ENSG00000147381 | SALPTTISF | 15 | HLA-A*30:02 |
| 4835 | MAGEA4 | ENSG00000147381 | SALPTTISF | 15 | HLA-A*31:01 |
| 4836 | MAGEA4 | ENSG00000147381 | SALPTTISF | 15 | HLA-A*32:01 |
| 4837 | MAGEA4 | ENSG00000147381 | SALPTTISF | 15 | HLA-A*33:01 |
| 4838 | MAGEA4 | ENSG00000147381 | SALPTTISF | 15 | HLA-A*33:03 |
| 4839 | MAGEA4 | ENSG00000147381 | SALPTTISF | 15 | HLA-A*68:01 |
| 4840 | MAGEA4 | ENSG00000147381 | SALPTTISF | 15 | HLA-B*07:02 |
| 4841 | MAGEA4 | ENSG00000147381 | SALPTTISF | 15 | HLA-B*08:01 |
| 4842 | MAGEA4 | ENSG00000147381 | SALPTTISF | 15 | HLA-B*15:01 |
| 4843 | MAGEA4 | ENSG00000147381 | SALPTTISF | 15 | HLA-B*15:03 |
| 4844 | MAGEA4 | ENSG00000147381 | SALPTTISF | 15 | HLA-B*18:01 |
| 4845 | MAGEA4 | ENSG00000147381 | SALPTTISF | 15 | HLA-B*27:02 |
| 4846 | MAGEA4 | ENSG00000147381 | SALPTTISF | 15 | HLA-B*35:01 |
| 4847 | MAGEA4 | ENSG00000147381 | SALPTTISF | 15 | HLA-B*35:03 |
| 4848 | MAGEA4 | ENSG00000147381 | SALPTTISF | 15 | HLA-B*37:01 |
| 4849 | MAGEA4 | ENSG00000147381 | SALPTTISF | 15 | HLA-B*38:01 |
| 4850 | MAGEA4 | ENSG00000147381 | SALPTTISF | 15 | HLA-B*39:01 |
| 4851 | MAGEA4 | ENSG00000147381 | SALPTTISF | 15 | HLA-B*44:02 |
| 4852 | MAGEA4 | ENSG00000147381 | SALPTTISF | 15 | HLA-B*44:03 |
| 4853 | MAGEA4 | ENSG00000147381 | SALPTTISF | 15 | HLA-B*46:01 |
| 4854 | MAGEA4 | ENSG00000147381 | SALPTTISF | 15 | HLA-B*51:01 |
| 4855 | MAGEA4 | ENSG00000147381 | SALPTTISF | 15 | HLA-B*54:01 |
| 4856 | MAGEA4 | ENSG00000147381 | SALPTTISF | 15 | HLA-B*55:01 |
| 4857 | MAGEA4 | ENSG00000147381 | SALPTTISF | 15 | HLA-B*56:01 |
| 4858 | MAGEA4 | ENSG00000147381 | SALPTTISF | 15 | HLA-B*57:01 |
| 4859 | MAGEA4 | ENSG00000147381 | SALPTTISF | 15 | HLA-B*58:01 |
| 4860 | MAGEA4 | ENSG00000147381 | SALPTTISF | 15 | HLA-C*01:02 |
| 4861 | MAGEA4 | ENSG00000147381 | SALPTTISF | 15 | HLA-C*02:02 |
| 4862 | MAGEA4 | ENSG00000147381 | SALPTTISF | 15 | HLA-C*03:03 |
| 4863 | MAGEA4 | ENSG00000147381 | SALPTTISF | 15 | HLA-C*03:04 |
| 4864 | MAGEA4 | ENSG00000147381 | SALPTTISF | 15 | HLA-C*04:01 |
| 4865 | MAGEA4 | ENSG00000147381 | SALPTTISF | 15 | HLA-C*05:01 |
| 4866 | MAGEA4 | ENSG00000147381 | SALPTTISF | 15 | HLA-C*07:02 |
| 4867 | MAGEA4 | ENSG00000147381 | SALPTTISF | 15 | HLA-C*07:04 |
| 4868 | MAGEA4 | ENSG00000147381 | SALPTTISF | 15 | HLA-C*07:06 |
| 4869 | MAGEA4 | ENSG00000147381 | SALPTTISF | 15 | HLA-C*12:03 |
| 4870 | MAGEA4 | ENSG00000147381 | SALPTTISF | 15 | HLA-C*14:02 |
| 4871 | MAGEA4 | ENSG00000147381 | SALPTTISF | 15 | HLA-C*16:01 |
| 4872 | MAGEA4 | ENSG00000147381 | SALPTTISF | 15 | HLA-C*16:02 |
| 4873 | MAGEA4 | ENSG00000147381 | SALPTTISF | 15 | HLA-C*16:04 |
| 4874 | MAGEA4 | ENSG00000147381 | SEEEIWEEL | 2349 | HLA-A*30:01 |
| 4875 | MAGEA4 | ENSG00000147381 | SEEEIWEEL | 2349 | HLA-B*40:01 |
| 4876 | MAGEA4 | ENSG00000147381 | SESLKMIF | 2350 | HLA-B*18:01 |
| 4877 | MAGEA4 | ENSG00000147381 | SESLKMIF | 2350 | HLA-B*37:01 |
| 4878 | MAGEA4 | ENSG00000147381 | SESLKMIF | 2350 | HLA-B*44:02 |
| 4879 | MAGEA4 | ENSG00000147381 | SESLKMIF | 2350 | HLA-B*44:03 |
| 4880 | MAGEA4 | ENSG00000147381 | SLFREALSNK | 2351 | HLA-A*03:01 |
| 4881 | MAGEA4 | ENSG00000147381 | SLFREALSNK | 2351 | HLA-A*03:02 |
| 4882 | MAGEA4 | ENSG00000147381 | SPDAESLFREA | 2352 | HLA-B*54:01 |
| 4883 | MAGEA4 | ENSG00000147381 | SPDAESLFREA | 2352 | HLA-B*55:01 |
| 4884 | MAGEA4 | ENSG00000147381 | SPDAESLFREA | 2352 | HLA-B*56:01 |
| 4885 | MAGEA4 | ENSG00000147381 | SPDAESLF | 2353 | HLA-B*55:01 |
| 4886 | MAGEA4 | ENSG00000147381 | SPDAESLF | 2353 | HLA-C*05:01 |
| 4887 | MAGEA4 | ENSG00000147381 | SPLVPGTLEEV | 2354 | HLA-B*56:01 |
| 4888 | MAGEA4 | ENSG00000147381 | SPLVPGTL | 2355 | HLA-B*07:02 |
| 4889 | MAGEA4 | ENSG00000147381 | SPLVPGTL | 2355 | HLA-B*08:01 |
| 4890 | MAGEA4 | ENSG00000147381 | SPLVPGTL | 2355 | HLA-C*07:02 |
| 4891 | MAGEA4 | ENSG00000147381 | SPQGASAL | 2356 | HLA-B*07:02 |
| 4892 | MAGEA4 | ENSG00000147381 | SPQGASAL | 2356 | HLA-C*07:02 |
| 4893 | MAGEA4 | ENSG00000147381 | SSPLVPGTL | 2357 | HLA-B*07:02 |
| 4894 | MAGEA4 | ENSG00000147381 | SSPLVPGTL | 2357 | HLA-B*46:01 |
| 4895 | MAGEA4 | ENSG00000147381 | SSPLVPGTL | 2357 | HLA-B*58:01 |
| 4896 | MAGEA4 | ENSG00000147381 | SSPLVPGTL | 2357 | HLA-C*01:02 |
| 4897 | MAGEA4 | ENSG00000147381 | SSPLVPGTL | 2357 | HLA-C*03:04 |
| 4898 | MAGEA4 | ENSG00000147381 | SSPLVPGTL | 2357 | HLA-C*07:02 |
| 4899 | MAGEA4 | ENSG00000147381 | STSPDAESLFR | 2358 | HLA-A*11:01 |
| 4900 | MAGEA4 | ENSG00000147381 | STSPDAESLFR | 2358 | HLA-A*68:01 |
| 4901 | MAGEA4 | ENSG00000147381 | STSPDAESLFR | 2358 | HLA-C*07:06 |
| 4902 | MAGEA4 | ENSG00000147381 | SYDGLLGNNQI | 2359 | HLA-A*23:01 |
| 4903 | MAGEA4 | ENSG00000147381 | SYDGLLGNNQI | 2359 | HLA-A*24:02 |
| 4904 | MAGEA4 | ENSG00000147381 | SYDGLLGNNQI | 2359 | HLA-B*35:03 |
| 4905 | MAGEA4 | ENSG00000147381 | SYDGLLGNNQI | 2359 | HLA-B*38:01 |
| 4906 | MAGEA4 | ENSG00000147381 | SYDGLLGNNQI | 2359 | HLA-C*04:01 |
| 4907 | MAGEA4 | ENSG00000147381 | SYDGLLGNN | 2360 | HLA-C*04:01 |
| 4908 | MAGEA4 | ENSG00000147381 | SYVKVLEHV | 2361 | HLA-A*23:01 |
| 4909 | MAGEA4 | ENSG00000147381 | SYVKVLEHV | 2361 | HLA-A*24:02 |
| 4910 | MAGEA4 | ENSG00000147381 | TLVTCLGLSY | 2362 | HLA-A*29:02 |
| 4911 | MAGEA4 | ENSG00000147381 | TQDWVQENYL | 2158 | HLA-B*38:01 |
| 4912 | MAGEA4 | ENSG00000147381 | TQDWVQENYL | 2158 | HLA-C*05:01 |
| 4913 | MAGEA4 | ENSG00000147381 | TQDWVQENY | 2159 | HLA-A*01:01 |
| 4914 | MAGEA4 | ENSG00000147381 | TQDWVQENY | 2159 | HLA-A*30:02 |
| 4915 | MAGEA4 | ENSG00000147381 | TQDWVQENY | 2159 | HLA-B*15:01 |
| 4916 | MAGEA4 | ENSG00000147381 | TQDWVQENY | 2159 | HLA-B*15:03 |
| 4917 | MAGEA4 | ENSG00000147381 | TQDWVQENY | 2159 | HLA-B*38:01 |
| 4918 | MAGEA4 | ENSG00000147381 | TQDWVQENY | 2159 | HLA-B*39:01 |
| 4919 | MAGEA4 | ENSG00000147381 | TQDWVQENY | 2159 | HLA-C*05:01 |
| 4920 | MAGEA4 | ENSG00000147381 | TSPDAESLFR | 2363 | HLA-A*68:01 |
| 4921 | MAGEA4 | ENSG00000147381 | TSPDAESLFR | 2363 | HLA-B*27:02 |
| 4922 | MAGEA4 | ENSG00000147381 | TSPDAESLF | 2364 | HLA-C*01:02 |
| 4923 | MAGEA4 | ENSG00000147381 | TSPDAESLF | 2364 | HLA-C*05:01 |
| 4924 | MAGEA4 | ENSG00000147381 | TSPDAESL | 2365 | HLA-C*01:02 |
| 4925 | MAGEA4 | ENSG00000147381 | TSYVKVLEHV | 2366 | HLA-A*68:02 |
| 4926 | MAGEA4 | ENSG00000147381 | TSYVKVLEH | 2367 | HLA-A*03:01 |
| 4927 | MAGEA4 | ENSG00000147381 | TSYVKVLEH | 2367 | HLA-A*11:01 |
| 4928 | MAGEA4 | ENSG00000147381 | TSYVKVLEH | 2367 | HLA-C*02:02 |
| 4929 | MAGEA4 | ENSG00000147381 | TSYVKVLEH | 2367 | HLA-C*07:06 |
| 4930 | MAGEA4 | ENSG00000147381 | TSYVKVLEH | 2367 | HLA-C*12:03 |
| 4931 | MAGEA4 | ENSG00000147381 | TTEEQEAAV | 2368 | HLA-C*05:01 |
| 4932 | MAGEA4 | ENSG00000147381 | TTISFTCWR | 2369 | HLA-A*31:01 |
| 4933 | MAGEA4 | ENSG00000147381 | TTISFTCW | 2370 | HLA-A*25:01 |
| 4934 | MAGEA4 | ENSG00000147381 | TTISFTCW | 2370 | HLA-B*57:01 |
| 4935 | MAGEA4 | ENSG00000147381 | TVYGEPRKL | 2371 | HLA-A*02:03 |
| 4936 | MAGEA4 | ENSG00000147381 | TVYGEPRKL | 2371 | HLA-A*03:01 |
| 4937 | MAGEA4 | ENSG00000147381 | TVYGEPRKL | 2371 | HLA-A*68:02 |
| 4938 | MAGEA4 | ENSG00000147381 | TVYGEPRKL | 2371 | HLA-C*02:02 |
| 4939 | MAGEA4 | ENSG00000147381 | TVYGEPRKL | 2371 | HLA-C*03:04 |
| 4940 | MAGEA4 | ENSG00000147381 | TVYGEPRKL | 2371 | HLA-C*06:02 |
| 4941 | MAGEA4 | ENSG00000147381 | TVYGEPRKL | 2371 | HLA-C*12:03 |
| 4942 | MAGEA4 | ENSG00000147381 | TYTLVTCL | 2372 | HLA-A*23:01 |
| 4943 | MAGEA4 | ENSG00000147381 | TYTLVTCL | 2372 | HLA-A*24:02 |
| 4944 | MAGEA4 | ENSG00000147381 | TYTLVTCL | 2372 | HLA-C*14:02 |
| 4945 | MAGEA4 | ENSG00000147381 | VDELAHFL | 2373 | HLA-B*37:01 |
| 4946 | MAGEA4 | ENSG00000147381 | VDELAHFL | 2373 | HLA-B*40:02 |
| 4947 | MAGEA4 | ENSG00000147381 | VDELAHFL | 2373 | HLA-C*07:04 |
| 4948 | MAGEA4 | ENSG00000147381 | VDPASNTY | 2374 | HLA-B*15:03 |
| 4949 | MAGEA4 | ENSG00000147381 | VDPASNTY | 2374 | HLA-B*37:01 |
| 4950 | MAGEA4 | ENSG00000147381 | VDPASNTY | 2374 | HLA-B*39:01 |
| 4951 | MAGEA4 | ENSG00000147381 | VDPASNTY | 2374 | HLA-C*01:02 |
| 4952 | MAGEA4 | ENSG00000147381 | VDPASNTY | 2374 | HLA-C*04:01 |
| 4953 | MAGEA4 | ENSG00000147381 | VDPASNTY | 2374 | HLA-C*07:01 |
| 4954 | MAGEA4 | ENSG00000147381 | VDPASNTY | 2374 | HLA-C*I2:03 |
| 4955 | MAGEA4 | ENSG00000147381 | VDPASNTY | 2374 | HLA-C*14:02 |
| 4956 | MAGEA4 | ENSG00000147381 | VDPASNTY | 2374 | HLA-C*16:02 |
| 4957 | MAGEA4 | ENSG00000147381 | VEAQEEALGLV | 2375 | HLA-B*49:01 |
| 4958 | MAGEA4 | ENSG00000147381 | VEAQEEALGL | 2376 | HLA-A*30:01 |
| 4959 | MAGEA4 | ENSG00000147381 | VEAQEEALGL | 2376 | HLA-B*40:01 |
| 4960 | MAGEA4 | ENSG00000147381 | VEAQEEAL | 2377 | HLA-A*30:01 |
| 4961 | MAGEA4 | ENSG00000147381 | VEAQEEAL | 2377 | HLA-B*40:01 |
| 4962 | MAGEA4 | ENSG00000147381 | VIFGKASESLK | 2378 | HLA-A*03:01 |
| 4963 | MAGEA4 | ENSG00000147381 | VIFGKASESLK | 2378 | HLA-A*03:02 |
| 4964 | MAGEA4 | ENSG00000147381 | VIFGKASESLK | 2378 | HLA-A*11:01 |
| 4965 | MAGEA4 | ENSG00000147381 | VIFGKASESL | 2379 | HLA-C*01:02 |
| 4966 | MAGEA4 | ENSG00000147381 | VKEVDPASNTY | 2380 | HLA-B*15:03 |
| 4967 | MAGEA4 | ENSG00000147381 | VNARVRIAY | 2381 | HLA-A*32:01 |
| 4968 | MAGEA4 | ENSG00000147381 | VNARVRIAY | 2381 | HLA-C*16:01 |
| 4969 | MAGEA4 | ENSG00000147381 | VPGSNPARYEF | 2382 | HLA-B*35:01 |
| 4970 | MAGEA4 | ENSG00000147381 | VPGSNPARY | 2383 | HLA-A*29:02 |
| 4971 | MAGEA4 | ENSG00000147381 | VPGSNPARY | 2383 | HLA-A*30:02 |
| 4972 | MAGEA4 | ENSG00000147381 | VPGSNPARY | 2383 | HLA-B*35:01 |
| 4973 | MAGEA4 | ENSG00000147381 | VPGSNPARY | 2383 | HLA-B*55:01 |
| 4974 | MAGEA4 | ENSG00000147381 | VPGTLEEV | 2384 | HLA-B*56:01 |
| 4975 | MAGEA4 | ENSG00000147381 | VQENYLEY | 2174 | HLA-A*01:01 |
| 4976 | MAGEA4 | ENSG00000147381 | VQENYLEY | 2174 | HLA-B*15:01 |
| 4977 | MAGEA4 | ENSG00000147381 | VQENYLEY | 2174 | HLA-B*15:03 |
| 4978 | MAGEA4 | ENSG00000147381 | VQENYLEY | 2174 | HLA-B*39:01 |
| 4979 | MAGEA4 | ENSG00000147381 | VQENYLEY | 2174 | HLA-C*07:01 |
| 4980 | MAGEA4 | ENSG00000147381 | VRIAYPSLR | 2385 | HLA-B*27:05 |
| 4981 | MAGEA4 | ENSG00000147381 | VTCLGLSY | 2386 | HLA-A*01:01 |
| 4982 | MAGEA4 | ENSG00000147381 | VTCLGLSY | 2386 | HLA-C*07:01 |
| 4983 | MAGEA4 | ENSG00000147381 | VTKAEMLER | 2387 | HLA-A*03:01 |
| 4984 | MAGEA4 | ENSG00000147381 | VTKAEMLER | 2387 | HLA-A*11:01 |
| 4985 | MAGEA4 | ENSG00000147381 | VTKAEMLER | 2387 | HLA-A*31:01 |
| 4986 | MAGEA4 | ENSG00000147381 | VTKAEMLER | 2387 | HLA-A*33:01 |
| 4987 | MAGEA4 | ENSG00000147381 | VTKAEMLER | 2387 | HLA-A*33:03 |
| 4988 | MAGEA4 | ENSG00000147381 | VTKAEMLER | 2387 | HLA-A*68:01 |
| 4989 | MAGEA4 | ENSG00000147381 | VTKAEMLER | 2387 | HLA-B*57:01 |
| 4990 | MAGEA4 | ENSG00000147381 | VTKAEMLER | 2387 | HLA-C*07:06 |
| 4991 | MAGEA4 | ENSG00000147381 | VYDGREHTVY | 2388 | HLA-A*24:02 |
| 4992 | MAGEA4 | ENSG00000147381 | VYDGREHTVY | 2388 | HLA-A*29:02 |
| 4993 | MAGEA4 | ENSG00000147381 | VYDGREHTVY | 2388 | HLA-B*35:01 |
| 4994 | MAGEA4 | ENSG00000147381 | VYDGREHTVY | 2388 | HLA-B*55:01 |
| 4995 | MAGEA4 | ENSG00000147381 | VYDGREHTVY | 2388 | HLA-C*04:01 |
| 4996 | MAGEA4 | ENSG00000147381 | VYDGREHTVY | 2388 | HLA-C*07:01 |
| 4997 | MAGEA4 | ENSG00000147381 | VYDGREHTV | 2389 | HLA-A*02:01 |
| 4998 | MAGEA4 | ENSG00000147381 | VYDGREHTV | 2389 | HLA-A*02:07 |
| 4999 | MAGEA4 | ENSG00000147381 | VYDGREHTV | 2389 | HLA-A*23:01 |
| 5000 | MAGEA4 | ENSG00000147381 | VYDGREHTV | 2389 | HLA-A*24:02 |
| 5001 | MAGEA4 | ENSG00000147381 | VYDGREHTV | 2389 | HLA-A*32:01 |
| 5002 | MAGEA4 | ENSG00000147381 | VYDGREHTV | 2389 | HLA-B*08:01 |
| 5003 | MAGEA4 | ENSG00000147381 | VYDGREHTV | 2389 | HLA-B*35:01 |
| 5004 | MAGEA4 | ENSG00000147381 | VYDGREHTV | 2389 | HLA-B*35:03 |
| 5005 | MAGEA4 | ENSG00000147381 | VYDGREHTV | 2389 | HLA-B*38:01 |
| 5006 | MAGEA4 | ENSG00000147381 | VYDGREHTV | 2389 | HLA-B*51:01 |
| 5007 | MAGEA4 | ENSG00000147381 | VYDGREHTV | 2389 | HLA-B*55:01 |
| 5008 | MAGEA4 | ENSG00000147381 | VYDGREHTV | 2389 | HLA-C*01:02 |
| 5009 | MAGEA4 | ENSG00000147381 | VYDGREHTV | 2389 | HLA-C*03:04 |
| 5010 | MAGEA4 | ENSG00000147381 | VYDGREHTV | 2389 | HLA-C*04:01 |
| 5011 | MAGEA4 | ENSG00000147381 | VYDGREHTV | 2389 | HLA-C*05:01 |
| 5012 | MAGEA4 | ENSG00000147381 | VYDGREHTV | 2389 | HLA-C*14:02 |
| 5013 | MAGEA4 | ENSG00000147381 | VYDGREHTV | 2389 | HLA-C*16:01 |
| 5014 | MAGEA4 | ENSG00000147381 | VYDGREHTV | 2389 | HLA-C*16:02 |
| 5015 | MAGEA4 | ENSG00000147381 | VYGEPRKLL | 2390 | HLA-A*23:01 |
| 5016 | MAGEA4 | ENSG00000147381 | VYGEPRKLL | 2390 | HLA-A*24:02 |
| 5017 | MAGEA4 | ENSG00000147381 | VYGEPRKL | 2391 | HLA-A*23:01 |
| 5018 | MAGEA4 | ENSG00000147381 | VYGEPRKL | 2391 | HLA-A*24:02 |
| 5019 | MAGEA4 | ENSG00000147381 | WEELGVMGV | 2392 | HLA-B*40:02 |
| 5020 | MAGEA4 | ENSG00000147381 | WEELGVMGV | 2392 | HLA-B*49:01 |
| 5021 | MAGEA4 | ENSG00000147381 | WVQENYLEY | 75 | HLA-A*01:01 |
| 5022 | MAGEA4 | ENSG00000147381 | WVQENYLEY | 75 | HLA-A*03:01 |
| 5023 | MAGEA4 | ENSG00000147381 | WVQENYLEY | 75 | HLA-A*25:01 |
| 5024 | MAGEA4 | ENSG00000147381 | WVQENYLEY | 75 | HLA-A*26:01 |
| 5025 | MAGEA4 | ENSG00000147381 | WVQENYLEY | 75 | HLA-A*29:02 |
| 5026 | MAGEA4 | ENSG00000147381 | WVQENYLEY | 75 | HLA-A*30:02 |
| 5027 | MAGEA4 | ENSG00000147381 | WVQENYLEY | 75 | HLA-B*15:01 |
| 5028 | MAGEA4 | ENSG00000147381 | WVQENYLEY | 75 | HLA-B*35:01 |
| 5029 | MAGEA4 | ENSG00000147381 | WVQENYLEY | 75 | HLA-B*46:01 |
| 5030 | MAGEA4 | ENSG00000147381 | WVQENYLEY | 75 | HLA-C*07:04 |
| 5031 | MAGEA4 | ENSG00000147381 | YDGLLGNNQIF | 2393 | HLA-C*07:01 |
| 5032 | MAGEA4 | ENSG00000147381 | YDGREHTVY | 2394 | HLA-A*01:01 |
| 5033 | MAGEA4 | ENSG00000147381 | YDGREHTVY | 2394 | HLA-A*29:02 |
| 5034 | MAGEA4 | ENSG00000147381 | YDGREHTVY | 2394 | HLA-C*07:01 |
| 5035 | MAGEA4 | ENSG00000147381 | YDGREHTV | 2395 | HLA-C*06:02 |
| 5036 | MAGEA4 | ENSG00000147381 | YDGREHTV | 2395 | HLA-C*07:01 |
| 5037 | MAGEA4 | ENSG00000147381 | YDGREHTV | 2395 | HLA-C*07:04 |
| 5038 | MAGEA4 | ENSG00000147381 | YEFLWGPRA | 2183 | HLA-A*02:04 |
| 5039 | MAGEA4 | ENSG00000147381 | YPSLREAALL | 2396 | HLA-B*07:02 |
| 5040 | MAGEA4 | ENSG00000147381 | YPSLREAAL | 2397 | HLA-B*07:02 |
| 5041 | MAGEA4 | ENSG00000147381 | YPSLREAAL | 2397 | HLA-B*08:01 |
| 5042 | MAGEA4 | ENSG00000147381 | YPSLREAAL | 2397 | HLA-B*35:01 |
| 5043 | MAGEA4 | ENSG00000147381 | YPSLREAAL | 2397 | HLA-B*35:03 |
| 5044 | MAGEA4 | ENSG00000147381 | YPSLREAAL | 2397 | HLA-B*37:01 |
| 5045 | MAGEA4 | ENSG00000147381 | YPSLREAAL | 2397 | HLA-B*39:01 |
| 5046 | MAGEA4 | ENSG00000147381 | YPSLREAAL | 2397 | HLA-B*40:01 |
| 5047 | MAGEA4 | ENSG00000147381 | YPSLREAAL | 2397 | HLA-B*51:01 |
| 5048 | MAGEA4 | ENSG00000147381 | YPSLREAAL | 2397 | HLA-B*54:01 |
| 5049 | MAGEA4 | ENSG00000147381 | YPSLREAAL | 2397 | HLA-B*55:01 |
| 5050 | MAGEA4 | ENSG00000147381 | YPSLREAAL | 2397 | HLA-B*56:01 |
| 5051 | MAGEA4 | ENSG00000147381 | YPSLREAAL | 2397 | HLA-C*01:02 |
| 5052 | MAGEA4 | ENSG00000147381 | YPSLREAAL | 2397 | HLA-C*03:03 |
| 5053 | MAGEA4 | ENSG00000147381 | YPSLREAAL | 2397 | HLA-C*03:04 |
| 5054 | MAGEA4 | ENSG00000147381 | YPSLREAAL | 2397 | HLA-C*07:02 |
| 5055 | MAGEA4 | ENSG00000147381 | YPSLREAAL | 2397 | HLA-C*14:02 |
| 5056 | MAGEA4 | ENSG00000147381 | YPSLREAAL | 2397 | HLA-C*16:01 |
| 5057 | MAGEA4 | ENSG00000147381 | YPSLREAA | 2398 | HLA-B*54:01 |
| 5058 | MAGEA4 | ENSG00000147381 | YPSLREAA | 2398 | HLA-B*55:01 |
| 5059 | MAGEA4 | ENSG00000147381 | YPSLREAA | 2398 | HLA-B*56:01 |
| 5060 | MAGEA4 | ENSG00000147381 | YRAKELVTK | 2399 | HLA-B*27:02 |
| 5061 | MAGEA4 | ENSG00000147381 | YRAKELVTK | 2399 | HLA-B*27:05 |
| 5062 | MAGEA4 | ENSG00000147381 | YRAKELVTK | 2399 | HLA-C*06:02 |
| 5063 | MAGEA4 | ENSG00000147381 | YRQVPGSNPAR | 2400 | HLA-B*27:05 |
| 5064 | MAGEA4 | ENSG00000147381 | YRQVPGSNP | 2401 | HLA-B*27:05 |
| 5065 | MAGEA4 | ENSG00000147381 | YVKVLEHVVR | 2402 | HLA-A*31:01 |
| 5066 | MAGEA4 | ENSG00000147381 | YVKVLEHVVR | 2402 | HLA-A*33:01 |
| 5067 | MAGEA4 | ENSG00000147381 | YVKVLEHVV | 2403 | HLA-A*02:01 |
| 5068 | MAGEA4 | ENSG00000147381 | YVKVLEHVV | 2403 | HLA-A*02:03 |
| 5069 | MAGEA4 | ENSG00000147381 | YVKVLEHVV | 2403 | HLA-A*02:04 |
| 5070 | MAGEA4 | ENSG00000147381 | YVKVLEHVV | 2403 | HLA-A*02:07 |
| 5071 | MAGEA4 | ENSG00000147381 | YVKVLEHVV | 2403 | HLA-A*24:02 |
| 5072 | MAGEA4 | ENSG00000147381 | YVKVLEHVV | 2403 | HLA-A*30:01 |
| 5073 | MAGEA4 | ENSG00000147381 | YVKVLEHVV | 2403 | HLA-A*32:01 |
| 5074 | MAGEA4 | ENSG00000147381 | YVKVLEHVV | 2403 | HLA-A*68:02 |
| 5075 | MAGEA4 | ENSG00000147381 | YVKVLEHVV | 2403 | HLA-B*08:01 |
| 5076 | MAGEA4 | ENSG00000147381 | YVKVLEHVV | 2403 | HLA-B*13:02 |
| 5077 | MAGEA4 | ENSG00000147381 | YVKVLEHVV | 2403 | HLA-B*40:02 |
| 5078 | MAGEA4 | ENSG00000147381 | YVKVLEHVV | 2403 | HLA-B*51:01 |
| 5079 | MAGEA4 | ENSG00000147381 | YVKVLEHVV | 2403 | HLA-B*54:01 |
| 5080 | MAGEA4 | ENSG00000147381 | YVKVLEHVV | 2403 | HLA-C*02:02 |
| 5081 | MAGEA4 | ENSG00000147381 | YVKVLEHVV | 2403 | HLA-C*04:01 |
| 5082 | MAGEA4 | ENSG00000147381 | YVKVLEHVV | 2403 | HLA-C*07:01 |
| 5083 | MAGEA4 | ENSG00000147381 | YVKVLEHVV | 2403 | HLA-C*07:04 |
| 5084 | MAGEA4 | ENSG00000147381 | YVKVLEHVV | 2403 | HLA-C*12:03 |
| 5085 | MAGEA4 | ENSG00000147381 | YVKVLEHVV | 2403 | HLA-C*16:02 |
| 5086 | MAGEA4 | ENSG00000147381 | YVKVLEHV | 2404 | HLA-A*02:03 |
| 5087 | MAGEA4 | ENSG00000147381 | YVKVLEHV | 2404 | HLA-B*08:01 |
| 5088 | MAGEA4 | ENSG00000147381 | YVKVLEHV | 2404 | HLA-B*54:01 |
| 5089 | NY-ESO1 | ENSG00000184033 | AADHRQLQL | 2405 | HLA-A*01:01 |
| 5090 | NY-ESO1 | ENSG00000184033 | AADHRQLQL | 2405 | HLA-A*02:07 |
| 5091 | NY-ESO1 | ENSG00000184033 | AADHRQLQL | 2405 | HLA-B*07:02 |
| 5092 | NY-ESO1 | ENSG00000184033 | AADHRQLQL | 2405 | HLA-B*08:01 |
| 5093 | NY-ESO1 | ENSG00000184033 | AADHRQLQL | 2405 | HLA-B*38:01 |
| 5094 | NY-ESO1 | ENSG00000184033 | AADHRQLQL | 2405 | HLA-B*40:01 |
| 5095 | NY-ESO1 | ENSG00000184033 | AADHRQLQL | 2405 | HLA-B*58:01 |
| 5096 | NY-ESO1 | ENSG00000184033 | AADHRQLQL | 2405 | HLA-C*01:02 |
| 5097 | NY-ESO1 | ENSG00000184033 | AADHRQLQL | 2405 | HLA-C*03:03 |
| 5098 | NY-ESO1 | ENSG00000184033 | AADHRQLQL | 2405 | HLA-C*03:04 |
| 5099 | NY-ESO1 | ENSG00000184033 | AADHRQLQL | 2405 | HLA-C*05:01 |
| 5100 | NY-ESO1 | ENSG00000184033 | AADHRQLQL | 2405 | HLA-C*07:02 |
| 5101 | NY-ESO1 | ENSG00000184033 | AADHRQLQL | 2405 | HLA-C*16:01 |
| 5102 | NY-ESO1 | ENSG00000184033 | AADHRQLQL | 2405 | HLA-C*16:02 |
| 5103 | NY-ESO1 | ENSG00000184033 | ADGPGGPGI | 2406 | HLA-A*30:01 |
| 5104 | NY-ESO1 | ENSG00000184033 | ADGPGGPGI | 2406 | HLA-B*37:01 |
| 5105 | NY-ESO1 | ENSG00000184033 | ADGPGGPGI | 2406 | HLA-B*38:01 |
| 5106 | NY-ESO1 | ENSG00000184033 | ADGPGGPGI | 2406 | HLA-B*44:02 |
| 5107 | NY-ESO1 | ENSG00000184033 | ADGPGGPGI | 2406 | HLA-B*44:03 |
| 5108 | NY-ESO1 | ENSG00000184033 | ADGPGGPGI | 2406 | HLA-B*49:01 |
| 5109 | NY-ESO1 | ENSG00000184033 | ADHRQLQL | 2407 | HLA-A*30:01 |
| 5110 | NY-ESO1 | ENSG00000184033 | ADHRQLQL | 2407 | HLA-B*37:01 |
| 5111 | NY-ESO1 | ENSG00000184033 | ADHRQLQL | 2407 | HLA-B*40:02 |
| 5112 | NY-ESO1 | ENSG00000184033 | ADHRQLQL | 2407 | HLA-C*06:02 |
| 5113 | NY-ESO1 | ENSG00000184033 | ADHRQLQL | 2407 | HLA-C*07:04 |
| 5114 | NY-ESO1 | ENSG00000184033 | AEGRGTGGST | 2408 | HLA-B*40:01 |
| 5115 | NY-ESO1 | ENSG00000184033 | AGAARASGPGG | 2409 | HLA-C*04:01 |
| 5116 | NY-ESO1 | ENSG00000184033 | AGAARASGP | 2410 | HLA-A*32:01 |
| 5117 | NY-ESO1 | ENSG00000184033 | AGAARASGP | 2410 | HLA-C*07:04 |
| 5118 | NY-ESO1 | ENSG00000184033 | AGAARASGP | 2410 | HLA-C*16:01 |
| 5119 | NY-ESO1 | ENSG00000184033 | AGAARASGP | 2410 | HLA-C*16:02 |
| 5120 | NY-ESO1 | ENSG00000184033 | AGATGGRGP | 2411 | HLA-C*16:01 |
| 5121 | NY-ESO1 | ENSG00000184033 | AMPFATPMEA | 2412 | HLA-A*02:01 |
| 5122 | NY-ESO1 | ENSG00000184033 | AMPFATPMEA | 2412 | HLA-A*02:03 |
| 5123 | NY-ESO1 | ENSG00000184033 | AMPFATPMEA | 2412 | HLA-A*02:07 |
| 5124 | NY-ESO1 | ENSG00000184033 | AMPFATPM | 2413 | HLA-C*01:02 |
| 5125 | NY-ESO1 | ENSG00000184033 | APPLPVPGVLL | 2414 | HLA-B*07:02 |
| 5126 | NY-ESO1 | ENSG00000184033 | APPLPVPGVL | 2415 | HLA-B*07:02 |
| 5127 | NY-ESO1 | ENSG00000184033 | APPLPVPGVL | 2415 | HLA-B*56:01 |
| 5128 | NY-ESO1 | ENSG00000184033 | APPLPVPGVL | 2415 | HLA-C*07:02 |
| 5129 | NY-ESO1 | ENSG00000184033 | APPLPVPGV | 2416 | HLA-A*02:07 |
| 5130 | NY-ESO1 | ENSG00000184033 | APPLPVPGV | 2416 | HLA-B*07:02 |
| 5131 | NY-ESO1 | ENSG00000184033 | APPLPVPGV | 2416 | HLA-B*13:02 |
| 5132 | NY-ESO1 | ENSG00000184033 | APPLPVPGV | 2416 | HLA-B*37:01 |
| 5133 | NY-ESO1 | ENSG00000184033 | APPLPVPGV | 2416 | HLA-B*51:01 |
| 5134 | NY-ESO1 | ENSG00000184033 | APPLPVPGV | 2416 | HLA-B*54:01 |
| 5135 | NY-ESO1 | ENSG00000184033 | APPLPVPGV | 2416 | HLA-B*55:01 |
| 5136 | NY-ESO1 | ENSG00000184033 | APPLPVPGV | 2416 | HLA-B*56:01 |
| 5137 | NY-ESO1 | ENSG00000184033 | APPLPVPGV | 2416 | HLA-C*04:01 |
| 5138 | NY-ESO1 | ENSG00000184033 | APPLPVPGV | 2416 | HLA-C*07:02 |
| 5139 | NY-ESO1 | ENSG00000184033 | APRGPHGGAAS | 2417 | HLA-B*07:02 |
| 5140 | NY-ESO1 | ENSG00000184033 | APRGPHGGAAS | 2417 | HLA-C*07:02 |
| 5141 | NY-ESO1 | ENSG00000184033 | APRGPHGGAA | 2418 | HLA-B*07:02 |
| 5142 | NY-ESO1 | ENSG00000184033 | APRGPHGGAA | 2418 | HLA-B*54:01 |
| 5143 | NY-ESO1 | ENSG00000184033 | APRGPHGGAA | 2418 | HLA-B*55:01 |
| 5144 | NY-ESO1 | ENSG00000184033 | APRGPHGGAA | 2418 | HLA-B*56:01 |
| 5145 | NY-ESO1 | ENSG00000184033 | APRGPHGGAA | 2418 | HLA-C*07:02 |
| 5146 | NY-ESO1 | ENSG00000184033 | APRGPHGGA | 2419 | HLA-B*07:02 |
| 5147 | NY-ESO1 | ENSG00000184033 | APRGPHGGA | 2419 | HLA-B*54:01 |
| 5148 | NY-ESO1 | ENSG00000184033 | APRGPHGGA | 2419 | HLA-B*55:01 |
| 5149 | NY-ESO1 | ENSG00000184033 | APRGPHGGA | 2419 | HLA-B*56:01 |
| 5150 | NY-ESO1 | ENSG00000184033 | APRGPHGGA | 2419 | HLA-C*07:02 |
| 5151 | NY-ESO1 | ENSG00000184033 | AQDAPPLPVPG | 2420 | HLA-B*27:05 |
| 5152 | NY-ESO1 | ENSG00000184033 | AQDAPPLPV | 2421 | HLA-A*01:01 |
| 5153 | NY-ESO1 | ENSG00000184033 | AQDAPPLPV | 2421 | HLA-A*02:01 |
| 5154 | NY-ESO1 | ENSG00000184033 | AQDAPPLPV | 2421 | HLA-A*02:03 |
| 5155 | NY-ESO1 | ENSG00000184033 | AQDAPPLPV | 2421 | HLA-A*02:07 |
| 5156 | NY-ESO1 | ENSG00000184033 | AQDAPPLPV | 2421 | HLA-A*03:01 |
| 5157 | NY-ESO1 | ENSG00000184033 | AQDAPPLPV | 2421 | HLA-A*11:01 |
| 5158 | NY-ESO1 | ENSG00000184033 | AQDAPPLPV | 2421 | HLA-A*30:01 |
| 5159 | NY-ESO1 | ENSG00000184033 | AQDAPPLPV | 2421 | HLA-B*13:02 |
| 5160 | NY-ESO1 | ENSG00000184033 | AQDAPPLPV | 2421 | HLA-B*27:05 |
| 5161 | NY-ESO1 | ENSG00000184033 | AQDAPPLPV | 2421 | HLA-B*37:01 |
| 5162 | NY-ESO1 | ENSG00000184033 | AQDAPPLPV | 2421 | HLA-B*38:01 |
| 5163 | NY-ESO1 | ENSG00000184033 | AQDAPPLPV | 2421 | HLA-B*39:01 |
| 5164 | NY-ESO1 | ENSG00000184033 | AQDAPPLPV | 2421 | HLA-B*49:01 |
| 5165 | NY-ESO1 | ENSG00000184033 | AQDAPPLPV | 2421 | HLA-B*55:01 |
| 5166 | NY-ESO1 | ENSG00000184033 | AQDAPPLPV | 2421 | HLA-C*02:02 |
| 5167 | NY-ESO1 | ENSG00000184033 | AQDAPPLPV | 2421 | HLA-C*03:03 |
| 5168 | NY-ESO1 | ENSG00000184033 | AQDAPPLPV | 2421 | HLA-C*03:04 |
| 5169 | NY-ESO1 | ENSG00000184033 | AQDAPPLPV | 2421 | HLA-C*05:01 |
| 5170 | NY-ESO1 | ENSG00000184033 | AQDAPPLPV | 2421 | HLA-C*06:02 |
| 5171 | NY-ESO1 | ENSG00000184033 | AQPPSGQRR | 2422 | HLA-A*31:01 |
| 5172 | NY-ESO1 | ENSG00000184033 | AQPPSGQRR | 2422 | HLA-A*32:01 |
| 5173 | NY-ESO1 | ENSG00000184033 | AQPPSGQRR | 2422 | HLA-B*27:05 |
| 5174 | NY-ESO1 | ENSG00000184033 | AQPPSGQRR | 2422 | HLA-C*06:02 |
| 5175 | NY-ESO1 | ENSG00000184033 | AQPPSGQRR | 2422 | HLA-C*07:02 |
| 5176 | NY-ESO1 | ENSG00000184033 | ARASGPGGGAP | 2423 | HLA-B*27:05 |
| 5177 | NY-ESO1 | ENSG00000184033 | ARASGPGGGAP | 2423 | HLA-B*39:01 |
| 5178 | NY-ESO1 | ENSG00000184033 | ARASGPGGGA | 2424 | HLA-B*27:05 |
| 5179 | NY-ESO1 | ENSG00000184033 | ARGPESRLLEF | 2425 | HLA-C*16:04 |
| 5180 | NY-ESO1 | ENSG00000184033 | ARGPESRLL | 2426 | HLA-B*07:02 |
| 5181 | NY-ESO1 | ENSG00000184033 | ARGPESRLL | 2426 | HLA-B*27:05 |
| 5182 | NY-ESO1 | ENSG00000184033 | ARGPESRLL | 2426 | HLA-C*06:02 |
| 5183 | NY-ESO1 | ENSG00000184033 | ARGPESRLL | 2426 | HLA-C*07:01 |
| 5184 | NY-ESO1 | ENSG00000184033 | ARGPESRLL | 2426 | HLA-C*07:02 |
| 5185 | NY-ESO1 | ENSG00000184033 | ASGPGGGAPR | 2427 | HLA-A*01:01 |
| 5186 | NY-ESO1 | ENSG00000184033 | ASGPGGGAPR | 2427 | HLA-A*03:02 |
| 5187 | NY-ESO1 | ENSG00000184033 | ASGPGGGAPR | 2427 | HLA-A*11:01 |
| 5188 | NY-ESO1 | ENSG00000184033 | ASGPGGGAPR | 2427 | HLA-A*30:02 |
| 5189 | NY-ESO1 | ENSG00000184033 | ASGPGGGAPR | 2427 | HLA-A*31:01 |
| 5190 | NY-ESO1 | ENSG00000184033 | ASGPGGGAPR | 2427 | HLA-A*33:01 |
| 5191 | NY-ESO1 | ENSG00000184033 | ASGPGGGAPR | 2427 | HLA-A*33:03 |
| 5192 | NY-ESO1 | ENSG00000184033 | ASGPGGGAPR | 2427 | HLA-A*68:01 |
| 5193 | NY-ESO1 | ENSG00000184033 | ASGPGGGAPR | 2427 | HLA-B*27:05 |
| 5194 | NY-ESO1 | ENSG00000184033 | ASGPGGGAPR | 2427 | HLA-C*01:02 |
| 5195 | NY-ESO1 | ENSG00000184033 | ASGPGGGAPR | 2427 | HLA-C*07:04 |
| 5196 | NY-ESO1 | ENSG00000184033 | ASGPGGGAPR | 2427 | HLA-C*07:06 |
| 5197 | NY-ESO1 | ENSG00000184033 | ATPMEAELARR | 2428 | HLA-A*11:01 |
| 5198 | NY-ESO1 | ENSG00000184033 | ATPMEAELAR | 2429 | HLA-C*07:06 |
| 5199 | NY-ESO1 | ENSG00000184033 | ATPMEAEL | 2430 | HLA-C*01:02 |
| 5200 | NY-ESO1 | ENSG00000184033 | DADGPGGPGI | 2431 | HLA-B*38:01 |
| 5201 | NY-ESO1 | ENSG00000184033 | DADGPGGPGI | 2431 | HLA-B*51:01 |
| 5202 | NY-ESO1 | ENSG00000184033 | DADGPGGPGI | 2431 | HLA-C*05:01 |
| 5203 | NY-ESO1 | ENSG00000184033 | DAPPLPVPGV | 2432 | HLA-A*26:01 |
| 5204 | NY-ESO1 | ENSG00000184033 | DAPPLPVPGV | 2432 | HLA-B*51:01 |
| 5205 | NY-ESO1 | ENSG00000184033 | DAPPLPVP | 2433 | HLA-B*51:01 |
| 5206 | NY-ESO1 | ENSG00000184033 | DGPGGPGI | 2434 | HLA-B*51:01 |
| 5207 | NY-ESO1 | ENSG00000184033 | DHRQLQLSI | 2435 | HLA-B*51:01 |
| 5208 | NY-ESO1 | ENSG00000184033 | EAELARRSL | 2436 | HLA-B*07:02 |
| 5209 | NY-ESO1 | ENSG00000184033 | EAELARRSL | 2436 | HLA-B*08:01 |
| 5210 | NY-ESO1 | ENSG00000184033 | EAELARRSL | 2436 | HLA-C*01:02 |
| 5211 | NY-ESO1 | ENSG00000184033 | EAELARRSL | 2436 | HLA-C*03:03 |
| 5212 | NY-ESO1 | ENSG00000184033 | EAELARRSL | 2436 | HLA-C*03:04 |
| 5213 | NY-ESO1 | ENSG00000184033 | EAELARRSL | 2436 | HLA-C*07:02 |
| 5214 | NY-ESO1 | ENSG00000184033 | EAELARRSL | 2436 | HLA-C*16:01 |
| 5215 | NY-ESO1 | ENSG00000184033 | EFTVSGNIL | 2437 | HLA-C*14:02 |
| 5216 | NY-ESO1 | ENSG00000184033 | EFYLAMPF | 2438 | HLA-C*07:01 |
| 5217 | NY-ESO1 | ENSG00000184033 | FATPMEAEL | 2439 | HLA-A*02:01 |
| 5218 | NY-ESO1 | ENSG00000184033 | FATPMEAEL | 2439 | HLA-A*23:01 |
| 5219 | NY-ESO1 | ENSG00000184033 | FATPMEAEL | 2439 | HLA-A*30:01 |
| 5220 | NY-ESO1 | ENSG00000184033 | FATPMEAEL | 2439 | HLA-A*32:01 |
| 5221 | NY-ESO1 | ENSG00000184033 | FATPMEAEL | 2439 | HLA-A*68:02 |
| 5222 | NY-ESO1 | ENSG00000184033 | FATPMEAEL | 2439 | HLA-B*13:02 |
| 5223 | NY-ESO1 | ENSG00000184033 | FATPMEAEL | 2439 | HLA-B*15:03 |
| 5224 | NY-ESO1 | ENSG00000184033 | FATPMEAEL | 2439 | HLA-B*27:05 |
| 5225 | NY-ESO1 | ENSG00000184033 | FATPMEAEL | 2439 | HLA-B*35:01 |
| 5226 | NY-ESO1 | ENSG00000184033 | FATPMEAEL | 2439 | HLA-B*35:03 |
| 5227 | NY-ESO1 | ENSG00000184033 | FATPMEAEL | 2439 | HLA-B*38:01 |
| 5228 | NY-ESO1 | ENSG00000184033 | FATPMEAEL | 2439 | HLA-B*39:01 |
| 5229 | NY-ESO1 | ENSG00000184033 | FATPMEAEL | 2439 | HLA-B*40:01 |
| 5230 | NY-ESO1 | ENSG00000184033 | FATPMEAEL | 2439 | HLA-B*46:01 |
| 5231 | NY-ESO1 | ENSG00000184033 | FATPMEAEL | 2439 | HLA-B*51:01 |
| 5232 | NY-ESO1 | ENSG00000184033 | FATPMEAEL | 2439 | HLA-B*54:01 |
| 5233 | NY-ESO1 | ENSG00000184033 | FATPMEAEL | 2439 | HLA-B*55:01 |
| 5234 | NY-ESO1 | ENSG00000184033 | FATPMEAEL | 2439 | HLA-B*58:01 |
| 5235 | NY-ESO1 | ENSG00000184033 | FATPMEAEL | 2439 | HLA-C*01:02 |
| 5236 | NY-ESO1 | ENSG00000184033 | FATPMEAEL | 2439 | HLA-C*02:02 |
| 5237 | NY-ESO1 | ENSG00000184033 | FATPMEAEL | 2439 | HLA-C*03:03 |
| 5238 | NY-ESO1 | ENSG00000184033 | FATPMEAEL | 2439 | HLA-C*03:04 |
| 5239 | NY-ESO1 | ENSG00000184033 | FATPMEAEL | 2439 | HLA-C*04:01 |
| 5240 | NY-ESO1 | ENSG00000184033 | FATPMEAEL | 2439 | HLA-C*05:01 |
| 5241 | NY-ESO1 | ENSG00000184033 | FATPMEAEL | 2439 | HLA-C*07:04 |
| 5242 | NY-ESO1 | ENSG00000184033 | FATPMEAEL | 2439 | HLA-C*07:06 |
| 5243 | NY-ESO1 | ENSG00000184033 | FATPMEAEL | 2439 | HLA-C*12:03 |
| 5244 | NY-ESO1 | ENSG00000184033 | FATPMEAEL | 2439 | HLA-C*14:02 |
| 5245 | NY-ESO1 | ENSG00000184033 | FATPMEAEL | 2439 | HLA-C*16:01 |
| 5246 | NY-ESO1 | ENSG00000184033 | FATPMEAEL | 2439 | HLA-C*16:02 |
| 5247 | NY-ESO1 | ENSG00000184033 | FATPMEAEL | 2439 | HLA-C*16:04 |
| 5248 | NY-ESO1 | ENSG00000184033 | FLAQPPSGQRR | 2440 | HLA-A*01:01 |
| 5249 | NY-ESO1 | ENSG00000184033 | FLAQPPSGQRR | 2440 | HLA-A*02:03 |
| 5250 | NY-ESO1 | ENSG00000184033 | FLAQPPSGQRR | 2440 | HLA-A*02:04 |
| 5251 | NY-ESO1 | ENSG00000184033 | FLAQPPSGQRR | 2440 | HLA-A*31:01 |
| 5252 | NY-ESO1 | ENSG00000184033 | FLAQPPSGQRR | 2440 | HLA-A*33:01 |
| 5253 | NY-ESO1 | ENSG00000184033 | FLAQPPSGQRR | 2440 | HLA-A*33:03 |
| 5254 | NY-ESO1 | ENSG00000184033 | FLAQPPSGQRR | 2440 | HLA-A*68:01 |
| 5255 | NY-ESO1 | ENSG00000184033 | FLAQPPSGQRR | 2440 | HLA-A*68:02 |
| 5256 | NY-ESO1 | ENSG00000184033 | FLAQPPSGQRR | 2440 | HLA-B*27:05 |
| 5257 | NY-ESO1 | ENSG00000184033 | FLAQPPSGQRR | 2440 | HLA-C*07:02 |
| 5258 | NY-ESO1 | ENSG00000184033 | FLAQPPSGQR | 2441 | HLA-A*68:01 |
| 5259 | NY-ESO1 | ENSG00000184033 | FLAQPPSGQ | 2442 | HLA-A*02:03 |
| 5260 | NY-ESO1 | ENSG00000184033 | FLAQPPSGQ | 2442 | HLA-A*32:01 |
| 5261 | NY-ESO1 | ENSG00000184033 | FLPVFLAQP | 2443 | HLA-A*02:07 |
| 5262 | NY-ESO1 | ENSG00000184033 | FTVSGNILTIR | 2444 | HLA-A*33:03 |
| 5263 | NY-ESO1 | ENSG00000184033 | FTVSGNILTIR | 2444 | HLA-A*68:01 |
| 5264 | NY-ESO1 | ENSG00000184033 | FTVSGNILTIR | 2444 | HLA-A*68:02 |
| 5265 | NY-ESO1 | ENSG00000184033 | FTVSGNILTI | 2445 | HLA-A*02:03 |
| 5266 | NY-ESO1 | ENSG00000184033 | FTVSGNILTI | 2445 | HLA-A*23:01 |
| 5267 | NY-ESO1 | ENSG00000184033 | FTVSGNILTI | 2445 | HLA-A*25:01 |
| 5268 | NY-ESO1 | ENSG00000184033 | FTVSGNILTI | 2445 | HLA-A*26:01 |
| 5269 | NY-ESO1 | ENSG00000184033 | FTVSGNILTI | 2445 | HLA-A*68:02 |
| 5270 | NY-ESO1 | ENSG00000184033 | FTVSGNILTI | 2445 | HLA-C*02:02 |
| 5271 | NY-ESO1 | ENSG00000184033 | FTVSGNIL | 2446 | HLA-B*39:01 |
| 5272 | NY-ESO1 | ENSG00000184033 | FTVSGNIL | 2446 | HLA-B*46:01 |
| 5273 | NY-ESO1 | ENSG00000184033 | FTVSGNIL | 2446 | HLA-C*02:02 |
| 5274 | NY-ESO1 | ENSG00000184033 | FTVSGNIL | 2446 | HLA-C*03:03 |
| 5275 | NY-ESO1 | ENSG00000184033 | FTVSGNIL | 2446 | HLA-C*03:04 |
| 5276 | NY-ESO1 | ENSG00000184033 | FTVSGNIL | 2446 | HLA-C*14:02 |
| 5277 | NY-ESO1 | ENSG00000184033 | GARGPESRL | 2447 | HLA-B*07:02 |
| 5278 | NY-ESO1 | ENSG00000184033 | GARGPESRL | 2447 | HLA-C*07:02 |
| 5279 | NY-ESO1 | ENSG00000184033 | GATGGRGP | 2448 | HLA-C*16:02 |
| 5280 | NY-ESO1 | ENSG00000184033 | GEAGATGGRGP | 2449 | HLA-C*06:02 |
| 5281 | NY-ESO1 | ENSG00000184033 | GEAGATGGRGP | 2449 | HLA-C*16:04 |
| 5282 | NY-ESO1 | ENSG00000184033 | GPESRLLEF | 2450 | HLA-B*07:02 |
| 5283 | NY-ESO1 | ENSG00000184033 | GPESRLLEF | 2450 | HLA-B*08:01 |
| 5284 | NY-ESO1 | ENSG00000184033 | GPESRLLEF | 2450 | HLA-B*35:01 |
| 5285 | NY-ESO1 | ENSG00000184033 | GPESRLLEF | 2450 | HLA-B*55:01 |
| 5286 | NY-ESO1 | ENSG00000184033 | GPESRLLEF | 2450 | HLA-C*07:02 |
| 5287 | NY-ESO1 | ENSG00000184033 | GPGGGAPRGP | 2451 | HLA-C*07:02 |
| 5288 | NY-ESO1 | ENSG00000184033 | GPHGGAASGL | 2452 | HLA-B*07:02 |
| 5289 | NY-ESO1 | ENSG00000184033 | GPHGGAASGL | 2452 | HLA-C*07:02 |
| 5290 | NY-ESO1 | ENSG00000184033 | GPRGAGAARAS | 2453 | HLA-B*07:02 |
| 5291 | NY-ESO1 | ENSG00000184033 | GPRGAGAARA | 2454 | HLA-B*07:02 |
| 5292 | NY-ESO1 | ENSG00000184033 | GPRGAGAARA | 2454 | HLA-B*56:01 |
| 5293 | NY-ESO1 | ENSG00000184033 | GPRGAGAARA | 2454 | HLA-C*07:02 |
| 5294 | NY-ESO1 | ENSG00000184033 | GPRGAGAAR | 2455 | HLA-A*33:03 |
| 5295 | NY-ESO1 | ENSG00000184033 | GPRGAGAAR | 2455 | HLA-B*07:02 |
| 5296 | NY-ESO1 | ENSG00000184033 | GPRGAGAAR | 2455 | HLA-C*07:02 |
| 5297 | NY-ESO1 | ENSG00000184033 | GRGPRGAGAAR | 2456 | HLA-B*27:05 |
| 5298 | NY-ESO1 | ENSG00000184033 | GVLLKEFTV | 2457 | HLA-A*02:01 |
| 5299 | NY-ESO1 | ENSG00000184033 | GVLLKEFTV | 2457 | HLA-A*02:04 |
| 5300 | NY-ESO1 | ENSG00000184033 | GVLLKEFTV | 2457 | HLA-B*13:02 |
| 5301 | NY-ESO1 | ENSG00000184033 | HGGAASGL | 2458 | HLA-C*07:04 |
| 5302 | NY-ESO1 | ENSG00000184033 | ILTIRLTAA | 2459 | HLA-A*02:01 |
| 5303 | NY-ESO1 | ENSG00000184033 | ILTIRLTAA | 2459 | HLA-A*02:03 |
| 5304 | NY-ESO1 | ENSG00000184033 | ILTIRLTAA | 2459 | HLA-A*02:04 |
| 5305 | NY-ESO1 | ENSG00000184033 | ILTIRLTAA | 2459 | HLA-B*08:01 |
| 5306 | NY-ESO1 | ENSG00000184033 | ILTIRLTAA | 2459 | HLA-B*55:01 |
| 5307 | NY-ESO1 | ENSG00000184033 | IPDGPGGNA | 2460 | HLA-B*07:02 |
| 5308 | NY-ESO1 | ENSG00000184033 | IPDGPGGNA | 2460 | HLA-B*55:01 |
| 5309 | NY-ESO1 | ENSG00000184033 | IPDGPGGNA | 2460 | HLA-B*56:01 |
| 5310 | NY-ESO1 | ENSG00000184033 | IPDGPGGNA | 2460 | HLA-C*05:01 |
| 5311 | NY-ESO1 | ENSG00000184033 | KEFTVSGNIL | 2461 | HLA-A*30:01 |
| 5312 | NY-ESO1 | ENSG00000184033 | KEFTVSGNIL | 2461 | HLA-B*27:05 |
| 5313 | NY-ESO1 | ENSG00000184033 | KEFTVSGNIL | 2461 | HLA-B*37:01 |
| 5314 | NY-ESO1 | ENSG00000184033 | KEFTVSGNIL | 2461 | HLA-B*40:01 |
| 5315 | NY-ESO1 | ENSG00000184033 | KEFTVSGNIL | 2461 | HLA-B*40:02 |
| 5316 | NY-ESO1 | ENSG00000184033 | KEFTVSGNIL | 2461 | HLA-B*44:02 |
| 5317 | NY-ESO1 | ENSG00000184033 | KEFTVSGNIL | 2461 | HLA-B*44:03 |
| 5318 | NY-ESO1 | ENSG00000184033 | KEFTVSGNIL | 2461 | HLA-B*49:01 |
| 5319 | NY-ESO1 | ENSG00000184033 | KEFTVSGNIL | 2461 | HLA-C*12:03 |
| 5320 | NY-ESO1 | ENSG00000184033 | KEFTVSGNIL | 2461 | HLA-C*16:04 |
| 5321 | NY-ESO1 | ENSG00000184033 | KEFTVSGNI | 2462 | HLA-A*30:01 |
| 5322 | NY-ESO1 | ENSG00000184033 | KEFTVSGNI | 2462 | HLA-B*13:02 |
| 5323 | NY-ESO1 | ENSG00000184033 | KEFTVSGNI | 2462 | HLA-B*37:01 |
| 5324 | NY-ESO1 | ENSG00000184033 | KEFTVSGNI | 2462 | HLA-B*40:01 |
| 5325 | NY-ESO1 | ENSG00000184033 | KEFTVSGNI | 2462 | HLA-B*40:02 |
| 5326 | NY-ESO1 | ENSG00000184033 | KEFTVSGNI | 2462 | HLA-B*44:02 |
| 5327 | NY-ESO1 | ENSG00000184033 | KEFTVSGNI | 2462 | HLA-B*44:03 |
| 5328 | NY-ESO1 | ENSG00000184033 | KEFTVSGNI | 2462 | HLA-B*49:01 |
| 5329 | NY-ESO1 | ENSG00000184033 | KEFTVSGNI | 2462 | HLA-C*16:04 |
| 5330 | NY-ESO1 | ENSG00000184033 | LAMPFATPMEA | 2463 | HLA-B*54:01 |
| 5331 | NY-ESO1 | ENSG00000184033 | LAMPFATPM | 2464 | HLA-A*11:01 |
| 5332 | NY-ESO1 | ENSG00000184033 | LAMPFATPM | 2464 | HLA-A*23:01 |
| 5333 | NY-ESO1 | ENSG00000184033 | LAMPFATPM | 2464 | HLA-A*24:02 |
| 5334 | NY-ESO1 | ENSG00000184033 | LAMPFATPM | 2464 | HLA-A*26:01 |
| 5335 | NY-ESO1 | ENSG00000184033 | LAMPFATPM | 2464 | HLA-B*07:02 |
| 5336 | NY-ESO1 | ENSG00000184033 | LAMPFATPM | 2464 | HLA-B*08:01 |
| 5337 | NY-ESO1 | ENSG00000184033 | LAMPFATPM | 2464 | HLA-B*15:01 |
| 5338 | NY-ESO1 | ENSG00000184033 | LAMPFATPM | 2464 | HLA-B*15:03 |
| 5339 | NY-ESO1 | ENSG00000184033 | LAMPFATPM | 2464 | HLA-B*18:01 |
| 5340 | NY-ESO1 | ENSG00000184033 | LAMPFATPM | 2464 | HLA-B*35:01 |
| 5341 | NY-ESO1 | ENSG00000184033 | LAMPFATPM | 2464 | HLA-B*35:03 |
| 5342 | NY-ESO1 | ENSG00000184033 | LAMPFATPM | 2464 | HLA-B*37:01 |
| 5343 | NY-ESO1 | ENSG00000184033 | LAMPFATPM | 2464 | HLA-B*39:01 |
| 5344 | NY-ESO1 | ENSG00000184033 | LAMPFATPM | 2464 | HLA-B*40:02 |
| 5345 | NY-ESO1 | ENSG00000184033 | LAMPFATPM | 2464 | HLA-B*46:01 |
| 5346 | NY-ESO1 | ENSG00000184033 | LAMPFATPM | 2464 | HLA-B*51:01 |
| 5347 | NY-ESO1 | ENSG00000184033 | LAMPFATPM | 2464 | HLA-B*54:01 |
| 5348 | NY-ESO1 | ENSG00000184033 | LAMPFATPM | 2464 | HLA-B*56:01 |
| 5349 | NY-ESO1 | ENSG00000184033 | LAMPFATPM | 2464 | HLA-B*57:01 |
| 5350 | NY-ESO1 | ENSG00000184033 | LAMPFATPM | 2464 | HLA-B*58:01 |
| 5351 | NY-ESO1 | ENSG00000184033 | LAMPFATPM | 2464 | HLA-C*01:02 |
| 5352 | NY-ESO1 | ENSG00000184033 | LAMPFATPM | 2464 | HLA-C*02:02 |
| 5353 | NY-ESO1 | ENSG00000184033 | LAMPFATPM | 2464 | HLA-C*03:03 |
| 5354 | NY-ESO1 | ENSG00000184033 | LAMPFATPM | 2464 | HLA-C*03:04 |
| 5355 | NY-ESO1 | ENSG00000184033 | LAMPFATPM | 2464 | HLA-C*04:01 |
| 5356 | NY-ESO1 | ENSG00000184033 | LAMPFATPM | 2464 | HLA-C*05:01 |
| 5357 | NY-ESO1 | ENSG00000184033 | LAMPFATPM | 2464 | HLA-C*06:02 |
| 5358 | NY-ESO1 | ENSG00000184033 | LAMPFATPM | 2464 | HLA-C*07:01 |
| 5359 | NY-ESO1 | ENSG00000184033 | LAMPFATPM | 2464 | HLA-C*07:04 |
| 5360 | NY-ESO1 | ENSG00000184033 | LAMPFATPM | 2464 | HLA-C*07:06 |
| 5361 | NY-ESO1 | ENSG00000184033 | LAMPFATPM | 2464 | HLA-C*12:03 |
| 5362 | NY-ESO1 | ENSG00000184033 | LAMPFATPM | 2464 | HLA-C*14:02 |
| 5363 | NY-ESO1 | ENSG00000184033 | LAMPFATPM | 2464 | HLA-C*16:01 |
| 5364 | NY-ESO1 | ENSG00000184033 | LAMPFATPM | 2464 | HLA-C*16:02 |
| 5365 | NY-ESO1 | ENSG00000184033 | LAMPFATPM | 2464 | HLA-C*16:04 |
| 5366 | NY-ESO1 | ENSG00000184033 | LAQDAPPLPV | 2465 | HLA-B*56:01 |
| 5367 | NY-ESO1 | ENSG00000184033 | LAQDAPPLPV | 2465 | HLA-C*03:03 |
| 5368 | NY-ESO1 | ENSG00000184033 | LAQDAPPLPV | 2465 | HLA-C*03:04 |
| 5369 | NY-ESO1 | ENSG00000184033 | LAQDAPPLPV | 2465 | HLA-C*16:04 |
| 5370 | NY-ESO1 | ENSG00000184033 | LAQDAPPL | 2466 | HLA-C*03:03 |
| 5371 | NY-ESO1 | ENSG00000184033 | LAQDAPPL | 2466 | HLA-C*03:04 |
| 5372 | NY-ESO1 | ENSG00000184033 | LAQPPSGQRR | 2467 | HLA-A*03:02 |
| 5373 | NY-ESO1 | ENSG00000184033 | LAQPPSGQRR | 2467 | HLA-A*11:01 |
| 5374 | NY-ESO1 | ENSG00000184033 | LAQPPSGQRR | 2467 | HLA-A*31:01 |
| 5375 | NY-ESO1 | ENSG00000184033 | LAQPPSGQRR | 2467 | HLA-A*33:01 |
| 5376 | NY-ESO1 | ENSG00000184033 | LAQPPSGQRR | 2467 | HLA-A*33:03 |
| 5377 | NY-ESO1 | ENSG00000184033 | LAQPPSGQRR | 2467 | HLA-A*68:01 |
| 5378 | NY-ESO1 | ENSG00000184033 | LAQPPSGQRR | 2467 | HLA-B*27:05 |
| 5379 | NY-ESO1 | ENSG00000184033 | LAQPPSGQRR | 2467 | HLA-B*57:01 |
| 5380 | NY-ESO1 | ENSG00000184033 | LAQPPSGQRR | 2467 | HLA-B*58:01 |
| 5381 | NY-ESO1 | ENSG00000184033 | LAQPPSGQRR | 2467 | HLA-C*06:02 |
| 5382 | NY-ESO1 | ENSG00000184033 | LAQPPSGQRR | 2467 | HLA-C*07:01 |
| 5383 | NY-ESO1 | ENSG00000184033 | LAQPPSGQRR | 2467 | HLA-C*07:02 |
| 5384 | NY-ESO1 | ENSG00000184033 | LAQPPSGQRR | 2467 | HLA-C*07:06 |
| 5385 | NY-ESO1 | ENSG00000184033 | LAQPPSGQRR | 2467 | HLA-C*16:02 |
| 5386 | NY-ESO1 | ENSG00000184033 | LAQPPSGQR | 2468 | HLA-A*03:02 |
| 5387 | NY-ESO1 | ENSG00000184033 | LAQPPSGQR | 2468 | HLA-A*11:01 |
| 5388 | NY-ESO1 | ENSG00000184033 | LAQPPSGQR | 2468 | HLA-A*31:01 |
| 5389 | NY-ESO1 | ENSG00000184033 | LAQPPSGQR | 2468 | HLA-A*33:01 |
| 5390 | NY-ESO1 | ENSG00000184033 | LAQPPSGQR | 2468 | HLA-A*33:03 |
| 5391 | NY-ESO1 | ENSG00000184033 | LAQPPSGQR | 2468 | HLA-A*68:01 |
| 5392 | NY-ESO1 | ENSG00000184033 | LAQPPSGQR | 2468 | HLA-B*27:05 |
| 5393 | NY-ESO1 | ENSG00000184033 | LAQPPSGQR | 2468 | HLA-C*07:06 |
| 5394 | NY-ESO1 | ENSG00000184033 | LAQPPSGQR | 2468 | HLA-C*16:02 |
| 5395 | NY-ESO1 | ENSG00000184033 | LPVFLAQPPSG | 2469 | HLA-B*54:01 |
| 5396 | NY-ESO1 | ENSG00000184033 | LPVFLAQPPS | 2470 | HLA-B*54:01 |
| 5397 | NY-ESO1 | ENSG00000184033 | LPVFLAQPP | 2471 | HLA-B*54:01 |
| 5398 | NY-ESO1 | ENSG00000184033 | LPVPGVLLKEF | 2472 | HLA-B*35:01 |
| 5399 | NY-ESO1 | ENSG00000184033 | LPVPGVLLK | 2473 | HLA-B*51:01 |
| 5400 | NY-ESO1 | ENSG00000184033 | LPVPGVLL | 2474 | HLA-B*35:01 |
| 5401 | NY-ESO1 | ENSG00000184033 | LPVPGVLL | 2474 | HLA-B*35:03 |
| 5402 | NY-ESO1 | ENSG00000184033 | LPVPGVLL | 2474 | HLA-B*51:01 |
| 5403 | NY-ESO1 | ENSG00000184033 | LPVPGVLL | 2474 | HLA-B*56:01 |
| 5404 | NY-ESO1 | ENSG00000184033 | LQLSISSCL | 2475 | HLA-B*27:05 |
| 5405 | NY-ESO1 | ENSG00000184033 | LTAADHRQL | 2476 | HLA-C*03:04 |
| 5406 | NY-ESO1 | ENSG00000184033 | LTAADHRQL | 2476 | HLA-C*06:02 |
| 5407 | NY-ESO1 | ENSG00000184033 | LTAADHRQL | 2476 | HLA-C*12:03 |
| 5408 | NY-ESO1 | ENSG00000184033 | LTAADHRQL | 2476 | HLA-C*16:01 |
| 5409 | NY-ESO1 | ENSG00000184033 | LTAADHRQL | 2476 | HLA-C*16:02 |
| 5410 | NY-ESO1 | ENSG00000184033 | MEAELARRSL | 2477 | HLA-A*30:01 |
| 5411 | NY-ESO1 | ENSG00000184033 | MEAELARRSL | 2477 | HLA-B*40:02 |
| 5412 | NY-ESO1 | ENSG00000184033 | MEAELARRSL | 2477 | HLA-B*44:02 |
| 5413 | NY-ESO1 | ENSG00000184033 | MEAELARRSL | 2477 | HLA-B*44:03 |
| 5414 | NY-ESO1 | ENSG00000184033 | MPFATPMEAEL | 2478 | HLA-A*68:02 |
| 5415 | NY-ESO1 | ENSG00000184033 | MPFATPMEAEL | 2478 | HLA-B*07:02 |
| 5416 | NY-ESO1 | ENSG00000184033 | MPFATPMEAEL | 2478 | HLA-B*35:01 |
| 5417 | NY-ESO1 | ENSG00000184033 | MPFATPMEAEL | 2478 | HLA-B*35:03 |
| 5418 | NY-ESO1 | ENSG00000184033 | MPFATPMEAEL | 2478 | HLA-B*51:01 |
| 5419 | NY-ESO1 | ENSG00000184033 | MPFATPMEAEL | 2478 | HLA-B*54:01 |
| 5420 | NY-ESO1 | ENSG00000184033 | MPFATPMEAEL | 2478 | HLA-B*55:01 |
| 5421 | NY-ESO1 | ENSG00000184033 | MPFATPMEAEL | 2478 | HLA-B*56:01 |
| 5422 | NY-ESO1 | ENSG00000184033 | MPFATPMEAEL | 2478 | HLA-C*03:03 |
| 5423 | NY-ESO1 | ENSG00000184033 | MPFATPMEAEL | 2478 | HLA-C*04:01 |
| 5424 | NY-ESO1 | ENSG00000184033 | MPFATPMEAEL | 2478 | HLA-C*07:01 |
| 5425 | NY-ESO1 | ENSG00000184033 | MPFATPMEAEL | 2478 | HLA-C*07:06 |
| 5426 | NY-ESO1 | ENSG00000184033 | MPFATPMEA | 2479 | HLA-A*02:01 |
| 5427 | NY-ESO1 | ENSG00000184033 | MPFATPMEA | 2479 | HLA-A*02:03 |
| 5428 | NY-ESO1 | ENSG00000184033 | MPFATPMEA | 2479 | HLA-B*07:02 |
| 5429 | NY-ESO1 | ENSG00000184033 | MPFATPMEA | 2479 | HLA-B*08:01 |
| 5430 | NY-ESO1 | ENSG00000184033 | MPFATPMEA | 2479 | HLA-B*35:01 |
| 5431 | NY-ESO1 | ENSG00000184033 | MPFATPMEA | 2479 | HLA-B*35:03 |
| 5432 | NY-ESO1 | ENSG00000184033 | MPFATPMEA | 2479 | HLA-B*51:01 |
| 5433 | NY-ESO1 | ENSG00000184033 | MPFATPMEA | 2479 | HLA-B*54:01 |
| 5434 | NY-ESO1 | ENSG00000184033 | MPFATPMEA | 2479 | HLA-B*55:01 |
| 5435 | NY-ESO1 | ENSG00000184033 | MPFATPMEA | 2479 | HLA-B*56:01 |
| 5436 | NY-ESO1 | ENSG00000184033 | MPFATPMEA | 2479 | HLA-C*02:02 |
| 5437 | NY-ESO1 | ENSG00000184033 | MPFATPMEA | 2479 | HLA-C*03:03 |
| 5438 | NY-ESO1 | ENSG00000184033 | MPFATPMEA | 2479 | HLA-C*04:01 |
| 5439 | NY-ESO1 | ENSG00000184033 | MPFATPMEA | 2479 | HLA-C*06:02 |
| 5440 | NY-ESO1 | ENSG00000184033 | MPFATPMEA | 2479 | HLA-C*07:02 |
| 5441 | NY-ESO1 | ENSG00000184033 | MPFATPMEA | 2479 | HLA-C*12:03 |
| 5442 | NY-ESO1 | ENSG00000184033 | MPFATPMEA | 2479 | HLA-C*14:02 |
| 5443 | NY-ESO1 | ENSG00000184033 | MPFATPMEA | 2479 | HLA-C*16:04 |
| 5444 | NY-ESO1 | ENSG00000184033 | MPFATPME | 2480 | HLA-B*54:01 |
| 5445 | NY-ESO1 | ENSG00000184033 | NILTIRLTAA | 2481 | HLA-A*02:03 |
| 5446 | NY-ESO1 | ENSG00000184033 | NILTIRLTA | 2482 | HLA-B*54:01 |
| 5447 | NY-ESO1 | ENSG00000184033 | NILTIRLTA | 2482 | HLA-C*16:01 |
| 5448 | NY-ESO1 | ENSG00000184033 | PFATPMEAEL | 2483 | HLA-C*07:02 |
| 5449 | NY-ESO1 | ENSG00000184033 | PGGGAPRGP | 2484 | HLA-C*07:02 |
| 5450 | NY-ESO1 | ENSG00000184033 | PLPVPGVLL | 2485 | HLA-A*24:02 |
| 5451 | NY-ESO1 | ENSG00000184033 | PPLPVPGVL | 2486 | HLA-B*07:02 |
| 5452 | NY-ESO1 | ENSG00000184033 | QDAPPLPVPG | 2487 | HLA-C*04:01 |
| 5453 | NY-ESO1 | ENSG00000184033 | QDAPPLPVPG | 2487 | HLA-C*06:02 |
| 5454 | NY-ESO1 | ENSG00000184033 | QDAPPLPVP | 2488 | HLA-B*27:05 |
| 5455 | NY-ESO1 | ENSG00000184033 | QDAPPLPVP | 2488 | HLA-B*40:02 |
| 5456 | NY-ESO1 | ENSG00000184033 | QDAPPLPVP | 2488 | HLA-C*04:01 |
| 5457 | NY-ESO1 | ENSG00000184033 | QDAPPLPVP | 2488 | HLA-C*06:02 |
| 5458 | NY-ESO1 | ENSG00000184033 | QDAPPLPVP | 2488 | HLA-C*07:01 |
| 5459 | NY-ESO1 | ENSG00000184033 | QDAPPLPVP | 2488 | HLA-C*07:02 |
| 5460 | NY-ESO1 | ENSG00000184033 | QDAPPLPVP | 2488 | HLA-C*07:04 |
| 5461 | NY-ESO1 | ENSG00000184033 | QDAPPLPVP | 2488 | HLA-C*12:03 |
| 5462 | NY-ESO1 | ENSG00000184033 | QDAPPLPVP | 2488 | HLA-C*16:02 |
| 5463 | NY-ESO1 | ENSG00000184033 | QDAPPLPV | 2489 | HLA-B*37:01 |
| 5464 | NY-ESO1 | ENSG00000184033 | RASGPGGGAPR | 2490 | HLA-A*31:01 |
| 5465 | NY-ESO1 | ENSG00000184033 | RASGPGGGAPR | 2490 | HLA-A*33:01 |
| 5466 | NY-ESO1 | ENSG00000184033 | RASGPGGGAPR | 2490 | HLA-A*33:03 |
| 5467 | NY-ESO1 | ENSG00000184033 | RASGPGGGAPR | 2490 | HLA-A*68:01 |
| 5468 | NY-ESO1 | ENSG00000184033 | RASGPGGGAPR | 2490 | HLA-B*27:05 |
| 5469 | NY-ESO1 | ENSG00000184033 | RASGPGGGAPR | 2490 | HLA-C*07:06 |
| 5470 | NY-ESO1 | ENSG00000184033 | RGAGAARASGP | 2491 | HLA-A*32:01 |
| 5471 | NY-ESO1 | ENSG00000184033 | RGPESRLLEF | 2492 | HLA-A*02:07 |
| 5472 | NY-ESO1 | ENSG00000184033 | RGPESRLLEF | 2492 | HLA-A*24:02 |
| 5473 | NY-ESO1 | ENSG00000184033 | RGPHGGAASGL | 2493 | HLA-C*01:02 |
| 5474 | NY-ESO1 | ENSG00000184033 | RGPRGAGAAR | 2494 | HLA-A*31:01 |
| 5475 | NY-ESO1 | ENSG00000184033 | RLLEFYLAM | 2495 | HLA-A*02:04 |
| 5476 | NY-ESO1 | ENSG00000184033 | RLLEFYLAM | 2495 | HLA-A*02:07 |
| 5477 | NY-ESO1 | ENSG00000184033 | SGNILTIRL | 2496 | HLA-A*02:04 |
| 5478 | NY-ESO1 | ENSG00000184033 | SGNILTIRL | 2496 | HLA-C*07:02 |
| 5479 | NY-ESO1 | ENSG00000184033 | SGPGGGAPRGP | 2497 | HLA-B*07:02 |
| 5480 | NY-ESO1 | ENSG00000184033 | SGPGGGAPRGP | 2497 | HLA-C*04:01 |
| 5481 | NY-ESO1 | ENSG00000184033 | SGPGGGAPRGP | 2497 | HLA-C*06:02 |
| 5482 | NY-ESO1 | ENSG00000184033 | SGPGGGAPRGP | 2497 | HLA-C*07:01 |
| 5483 | NY-ESO1 | ENSG00000184033 | SGPGGGAPRGP | 2497 | HLA-C*07:02 |
| 5484 | NY-ESO1 | ENSG00000184033 | SGPGGGAPRGP | 2497 | HLA-C*16:02 |
| 5485 | NY-ESO1 | ENSG00000184033 | SGPGGGAPR | 2498 | HLA-A*68:01 |
| 5486 | NY-ESO1 | ENSG00000184033 | SGPGGGAPR | 2498 | HLA-C*01:02 |
| 5487 | NY-ESO1 | ENSG00000184033 | SGPGGGAPR | 2498 | HLA-C*07:06 |
| 5488 | NY-ESO1 | ENSG00000184033 | SGPGGGAP | 2499 | HLA-C*01:02 |
| 5489 | NY-ESO1 | ENSG00000184033 | SISSCLQQL | 2500 | HLA-A*02:04 |
| 5490 | NY-ESO1 | ENSG00000184033 | SISSCLQQL | 2500 | HLA-A*24:02 |
| 5491 | NY-ESO1 | ENSG00000184033 | SISSCLQQL | 2500 | HLA-A*68:02 |
| 5492 | NY-ESO1 | ENSG00000184033 | SISSCLQQL | 2500 | HLA-C*07:04 |
| 5493 | NY-ESO1 | ENSG00000184033 | SLAQDAPPLPV | 2501 | HLA-A*02:01 |
| 5494 | NY-ESO1 | ENSG00000184033 | SLAQDAPPLPV | 2501 | HLA-A*02:03 |
| 5495 | NY-ESO1 | ENSG00000184033 | SLAQDAPPL | 2502 | HLA-A*02:01 |
| 5496 | NY-ESO1 | ENSG00000184033 | SLAQDAPPL | 2502 | HLA-A*02:04 |
| 5497 | NY-ESO1 | ENSG00000184033 | SLAQDAPPL | 2502 | HLA-B*35:03 |
| 5498 | NY-ESO1 | ENSG00000184033 | SLAQDAPPL | 2502 | HLA-C*01:02 |
| 5499 | NY-ESO1 | ENSG00000184033 | SLLMVVITQC | 2503 | HLA-A*02:01 |
| 5500 | NY-ESO1 | ENSG00000184033 | SLLMVVITQC | 2503 | HLA-A*02:04 |
| 5501 | NY-ESO1 | ENSG00000184033 | TAADHRQLQL | 2504 | HLA-A*30:01 |
| 5502 | NY-ESO1 | ENSG00000184033 | TAADHRQLQL | 2504 | HLA-B*07:02 |
| 5503 | NY-ESO1 | ENSG00000184033 | TAADHRQLQL | 2504 | HLA-C*07:02 |
| 5504 | NY-ESO1 | ENSG00000184033 | TAADHRQLQL | 2504 | HLA-C*16:01 |
| 5505 | NY-ESO1 | ENSG00000184033 | TAADHRQLQL | 2504 | HLA-C*16:02 |
| 5506 | NY-ESO1 | ENSG00000184033 | TAADHRQL | 2505 | HLA-B*07:02 |
| 5507 | NY-ESO1 | ENSG00000184033 | TAADHRQL | 2505 | HLA-B*08:01 |
| 5508 | NY-ESO1 | ENSG00000184033 | TAADHRQL | 2505 | HLA-B*35:03 |
| 5509 | NY-ESO1 | ENSG00000184033 | TAADHRQL | 2505 | HLA-C*03:03 |
| 5510 | NY-ESO1 | ENSG00000184033 | TAADHRQL | 2505 | HLA-C*03:04 |
| 5511 | NY-ESO1 | ENSG00000184033 | TAADHRQL | 2505 | HLA-C*06:02 |
| 5512 | NY-ESO1 | ENSG00000184033 | TAADHRQL | 2505 | HLA-C*12:03 |
| 5513 | NY-ESO1 | ENSG00000184033 | TAADHRQL | 2505 | HLA-C*16:01 |
| 5514 | NY-ESO1 | ENSG00000184033 | TAADHRQL | 2505 | HLA-C*16:02 |
| 5515 | NY-ESO1 | ENSG00000184033 | TPMEAELARR | 2506 | HLA-A*33:03 |
| 5516 | NY-ESO1 | ENSG00000184033 | TPMEAELARR | 2506 | HLA-A*68:01 |
| 5517 | NY-ESO1 | ENSG00000184033 | TPMEAELARR | 2506 | HLA-C*07:06 |
| 5518 | NY-ESO1 | ENSG00000184033 | TPMEAELAR | 2507 | HLA-A*33:03 |
| 5519 | NY-ESO1 | ENSG00000184033 | TPMEAELAR | 2507 | HLA-A*68:01 |
| 5520 | NY-ESO1 | ENSG00000184033 | TPMEAELAR | 2507 | HLA-B*35:01 |
| 5521 | NY-ESO1 | ENSG00000184033 | TPMEAELAR | 2507 | HLA-B*35:03 |
| 5522 | NY-ESO1 | ENSG00000184033 | TPMEAELAR | 2507 | HLA-B*54:01 |
| 5523 | NY-ESO1 | ENSG00000184033 | TPMEAELAR | 2507 | HLA-B*55:01 |
| 5524 | NY-ESO1 | ENSG00000184033 | TPMEAELAR | 2507 | HLA-B*56:01 |
| 5525 | NY-ESO1 | ENSG00000184033 | TPMEAELAR | 2507 | HLA-C*03:03 |
| 5526 | NY-ESO1 | ENSG00000184033 | TPMEAELAR | 2507 | HLA-C*07:06 |
| 5527 | NY-ESO1 | ENSG00000184033 | TPMEAELA | 2508 | HLA-B*56:01 |
| 5528 | NY-ESO1 | ENSG00000184033 | TVSGNILTIRL | 2509 | HLA-A*68:02 |
| 5529 | NY-ESO1 | ENSG00000184033 | TVSGNILTIR | 2510 | HLA-A*31:01 |
| 5530 | NY-ESO1 | ENSG00000184033 | TVSGNILTIR | 2510 | HLA-A*33:03 |
| 5531 | NY-ESO1 | ENSG00000184033 | TVSGNILTIR | 2510 | HLA-A*68:01 |
| 5532 | NY-ESO1 | ENSG00000184033 | TVSGNILTIR | 2510 | HLA-A*68:02 |
| 5533 | NY-ESO1 | ENSG00000184033 | TVSGNILTIR | 2510 | HLA-C*07:06 |
| 5534 | NY-ESO1 | ENSG00000184033 | TVSGNILTI | 2511 | HLA-A*02:01 |
| 5535 | NY-ESO1 | ENSG00000184033 | TVSGNILTI | 2511 | HLA-A*02:03 |
| 5536 | NY-ESO1 | ENSG00000184033 | TVSGNILTI | 2511 | HLA-A*02:04 |
| 5537 | NY-ESO1 | ENSG00000184033 | TVSGNILTI | 2511 | HLA-A*02:07 |
| 5538 | NY-ESO1 | ENSG00000184033 | TVSGNILTI | 2511 | HLA-A*03:01 |
| 5539 | NY-ESO1 | ENSG00000184033 | TVSGNILTI | 2511 | HLA-A*11:01 |
| 5540 | NY-ESO1 | ENSG00000184033 | TVSGNILTI | 2511 | HLA-A*23:01 |
| 5541 | NY-ESO1 | ENSG00000184033 | TVSGNILTI | 2511 | HLA-A*25:01 |
| 5542 | NY-ESO1 | ENSG00000184033 | TVSGNILTI | 2511 | HLA-A*26:01 |
| 5543 | NY-ESO1 | ENSG00000184033 | TVSGNILTI | 2511 | HLA-A*30:01 |
| 5544 | NY-ESO1 | ENSG00000184033 | TVSGNILTI | 2511 | HLA-A*32:01 |
| 5545 | NY-ESO1 | ENSG00000184033 | TVSGNILTI | 2511 | HLA-A*68:01 |
| 5546 | NY-ESO1 | ENSG00000184033 | TVSGNILTI | 2511 | HLA-A*68:02 |
| 5547 | NY-ESO1 | ENSG00000184033 | TVSGNILTI | 2511 | HLA-B*13:02 |
| 5548 | NY-ESO1 | ENSG00000184033 | TVSGNILTI | 2511 | HLA-B*27:05 |
| 5549 | NY-ESO1 | ENSG00000184033 | TVSGNILTI | 2511 | HLA-B*35:01 |
| 5550 | NY-ESO1 | ENSG00000184033 | TVSGNILTI | 2511 | HLA-B*35:03 |
| 5551 | NY-ESO1 | ENSG00000184033 | TVSGNILTI | 2511 | HLA-B*38:01 |
| 5552 | NY-ESO1 | ENSG00000184033 | TVSGNILTI | 2511 | HLA-B*39:01 |
| 5553 | NY-ESO1 | ENSG00000184033 | TVSGNILTI | 2511 | HLA-B*40:02 |
| 5554 | NY-ESO1 | ENSG00000184033 | TVSGNILTI | 2511 | HLA-B*49:01 |
| 5555 | NY-ESO1 | ENSG00000184033 | TVSGNILTI | 2511 | HLA-B*51:01 |
| 5556 | NY-ESO1 | ENSG00000184033 | TVSGNILTI | 2511 | HLA-B*55:01 |
| 5557 | NY-ESO1 | ENSG00000184033 | TVSGNILTI | 2511 | HLA-B*58:01 |
| 5558 | NY-ESO1 | ENSG00000184033 | TVSGNILTI | 2511 | HLA-C*02:02 |
| 5559 | NY-ESO1 | ENSG00000184033 | TVSGNILTI | 2511 | HLA-C*03:04 |
| 5560 | NY-ESO1 | ENSG00000184033 | TVSGNILTI | 2511 | HLA-C*04:01 |
| 5561 | NY-ESO1 | ENSG00000184033 | TVSGNILTI | 2511 | HLA-C*06:02 |
| 5562 | NY-ESO1 | ENSG00000184033 | TVSGNILTI | 2511 | HLA-C*07:04 |
| 5563 | NY-ESO1 | ENSG00000184033 | TVSGNILTI | 2511 | HLA-C*07:06 |
| 5564 | NY-ESO1 | ENSG00000184033 | TVSGNILTI | 2511 | HLA-C*12:03 |
| 5565 | NY-ESO1 | ENSG00000184033 | TVSGNILTI | 2511 | HLA-C*16:02 |
| 5566 | NY-ESO1 | ENSG00000184033 | VLLKEFTV | 2512 | HLA-A*02:01 |
| 5567 | NY-ESO1 | ENSG00000184033 | VLLKEFTV | 2512 | HLA-A*02:04 |
| 5568 | NY-ESO1 | ENSG00000184033 | VSGNILTIR | 2513 | HLA-A*31:01 |
| 5569 | NY-ESO1 | ENSG00000184033 | VSGNILTIR | 2513 | HLA-A*33:03 |
| 5570 | NY-ESO1 | ENSG00000184033 | VSGNILTIR | 2513 | HLA-A*68:01 |
| 5571 | NY-ESO1 | ENSG00000184033 | VSGNILTIR | 2513 | HLA-C*07:06 |
| 5572 | NY-ESO1 | ENSG00000184033 | VSGNILTI | 2514 | HLA-A*23:01 |
| 5573 | NY-ESO1 | ENSG00000184033 | VSGNILTI | 2514 | HLA-B*13:02 |
| 5574 | NY-ESO1 | ENSG00000184033 | VSGNILTI | 2514 | HLA-B*51:01 |
| 5575 | NY-ESO1 | ENSG00000184033 | VSGNILTI | 2514 | HLA-B*58:01 |
| 5576 | NY-ESO1 | ENSG00000184033 | YLAMPFATPM | 2515 | HLA-C*07:01 |
| 5577 | WT1 | ENSG00000184937 | AAGSSSSVKW | 2516 | HLA-A*25:01 |
| 5578 | WT1 | ENSG00000184937 | AAGSSSSVKW | 2516 | HLA-B*27:02 |
| 5579 | WT1 | ENSG00000184937 | AAGSSSSVKW | 2516 | HLA-B*44:02 |
| 5580 | WT1 | ENSG00000184937 | AAGSSSSVKW | 2516 | HLA-B*44:03 |
| 5581 | WT1 | ENSG00000184937 | AAGSSSSVKW | 2516 | HLA-B*58:01 |
| 5582 | WT1 | ENSG00000184937 | AAGSSSSVKW | 2516 | HLA-C*16:04 |
| 5583 | WT1 | ENSG00000184937 | AAGSSSSVK | 2517 | HLA-A*03:02 |
| 5584 | WT1 | ENSG00000184937 | AAQFPNHSFK | 2518 | HLA-A*03:01 |
| 5585 | WT1 | ENSG00000184937 | AAQFPNHSFK | 2518 | HLA-A*03:02 |
| 5586 | WT1 | ENSG00000184937 | AAQFPNHSFK | 2518 | HLA-A*11:01 |
| 5587 | WT1 | ENSG00000184937 | AAQFPNHSFK | 2518 | HLA-B*27:02 |
| 5588 | WT1 | ENSG00000184937 | AAQFPNHSF | 2519 | HLA-A*23:01 |
| 5589 | WT1 | ENSG00000184937 | AAQFPNHSF | 2519 | HLA-A*24:02 |
| 5590 | WT1 | ENSG00000184937 | AAQFPNHSF | 2519 | HLA-A*30:02 |
| 5591 | WT1 | ENSG00000184937 | AAQFPNHSF | 2519 | HLA-A*32:01 |
| 5592 | WT1 | ENSG00000184937 | AAQFPNHSF | 2519 | HLA-B*07:02 |
| 5593 | WT1 | ENSG00000184937 | AAQFPNHSF | 2519 | HLA-B*15:01 |
| 5594 | WT1 | ENSG00000184937 | AAQFPNHSF | 2519 | HLA-B*15:03 |
| 5595 | WT1 | ENSG00000184937 | AAQFPNHSF | 2519 | HLA-B*35:01 |
| 5596 | WT1 | ENSG00000184937 | AAQFPNHSF | 2519 | HLA-B*37:01 |
| 5597 | WT1 | ENSG00000184937 | AAQFPNHSF | 2519 | HLA-B*39:01 |
| 5598 | WT1 | ENSG00000184937 | AAQFPNHSF | 2519 | HLA-B*44:02 |
| 5599 | WT1 | ENSG00000184937 | AAQFPNHSF | 2519 | HLA-B*46:01 |
| 5600 | WT1 | ENSG00000184937 | AAQFPNHSF | 2519 | HLA-B*57:01 |
| 5601 | WT1 | ENSG00000184937 | AAQFPNHSF | 2519 | HLA-B*58:01 |
| 5602 | WT1 | ENSG00000184937 | AAQFPNHSF | 2519 | HLA-C*01:02 |
| 5603 | WT1 | ENSG00000184937 | AAQFPNHSF | 2519 | HLA-C*02:02 |
| 5604 | WT1 | ENSG00000184937 | AAQFPNHSF | 2519 | HLA-C*03:03 |
| 5605 | WT1 | ENSG00000184937 | AAQFPNHSF | 2519 | HLA-C*03:04 |
| 5606 | WT1 | ENSG00000184937 | AAQFPNHSF | 2519 | HLA-C*05:01 |
| 5607 | WT1 | ENSG00000184937 | AAQFPNHSF | 2519 | HLA-C*07:04 |
| 5608 | WT1 | ENSG00000184937 | AAQFPNHSF | 2519 | HLA-C*12:03 |
| 5609 | WT1 | ENSG00000184937 | AAQFPNHSF | 2519 | HLA-C*14:02 |
| 5610 | WT1 | ENSG00000184937 | AAQFPNHSF | 2519 | HLA-C*16:01 |
| 5611 | WT1 | ENSG00000184937 | AAQFPNHSF | 2519 | HLA-C*16:02 |
| 5612 | WT1 | ENSG00000184937 | AAQFPNHSF | 2519 | HLA-C*16:04 |
| 5613 | WT1 | ENSG00000184937 | AEPHEEQCL | 2520 | HLA-B*40:01 |
| 5614 | WT1 | ENSG00000184937 | AEPHEEQCL | 2520 | HLA-B*44:03 |
| 5615 | WT1 | ENSG00000184937 | AGSSSSVKW | 2521 | HLA-A*32:01 |
| 5616 | WT1 | ENSG00000184937 | AGSSSSVKW | 2521 | HLA-B*27:02 |
| 5617 | WT1 | ENSG00000184937 | AGSSSSVKW | 2521 | HLA-B*44:02 |
| 5618 | WT1 | ENSG00000184937 | AGSSSSVKW | 2521 | HLA-B*44:03 |
| 5619 | WT1 | ENSG00000184937 | AGSSSSVKW | 2521 | HLA-B*58:01 |
| 5620 | WT1 | ENSG00000184937 | AGSSSSVKW | 2521 | HLA-C*12:03 |
| 5621 | WT1 | ENSG00000184937 | AGSSSSVKW | 2521 | HLA-C*16:01 |
| 5622 | WT1 | ENSG00000184937 | AGSSSSVKW | 2521 | HLA-C*16:04 |
| 5623 | WT1 | ENSG00000184937 | AIRNQGYSTV | 2522 | HLA-A*02:03 |
| 5624 | WT1 | ENSG00000184937 | ALLPAVPSL | 2523 | HLA-A*02:01 |
| 5625 | WT1 | ENSG00000184937 | ALLPAVPSL | 2523 | HLA-A*02:03 |
| 5626 | WT1 | ENSG00000184937 | ALLPAVPSL | 2523 | HLA-A*02:04 |
| 5627 | WT1 | ENSG00000184937 | ALLPAVPSL | 2523 | HLA-A*02:07 |
| 5628 | WT1 | ENSG00000184937 | ALLPAVPSL | 2523 | HLA-A*03:01 |
| 5629 | WT1 | ENSG00000184937 | ALLPAVPSL | 2523 | HLA-A*03:02 |
| 5630 | WT1 | ENSG00000184937 | ALLPAVPSL | 2523 | HLA-A*23:01 |
| 5631 | WT1 | ENSG00000184937 | ALLPAVPSL | 2523 | HLA-A*24:02 |
| 5632 | WT1 | ENSG00000184937 | ALLPAVPSL | 2523 | HLA-A*25:01 |
| 5633 | WT1 | ENSG00000184937 | ALLPAVPSL | 2523 | HLA-A*29:02 |
| 5634 | WT1 | ENSG00000184937 | ALLPAVPSL | 2523 | HLA-A*30:01 |
| 5635 | WT1 | ENSG00000184937 | ALLPAVPSL | 2523 | HLA-A*31:01 |
| 5636 | WT1 | ENSG00000184937 | ALLPAVPSL | 2523 | HLA-A*32:01 |
| 5637 | WT1 | ENSG00000184937 | ALLPAVPSL | 2523 | HLA-B*07:02 |
| 5638 | WT1 | ENSG00000184937 | ALLPAVPSL | 2523 | HLA-B*13:02 |
| 5639 | WT1 | ENSG00000184937 | ALLPAVPSL | 2523 | HLA-B*15:01 |
| 5640 | WT1 | ENSG00000184937 | ALLPAVPSL | 2523 | HLA-B*15:03 |
| 5641 | WT1 | ENSG00000184937 | ALLPAVPSL | 2523 | HLA-B*37:01 |
| 5642 | WT1 | ENSG00000184937 | ALLPAVPSL | 2523 | HLA-B*40:01 |
| 5643 | WT1 | ENSG00000184937 | ALLPAVPSL | 2523 | HLA-B*40:02 |
| 5644 | WT1 | ENSG00000184937 | ALLPAVPSL | 2523 | HLA-B*46:01 |
| 5645 | WT1 | ENSG00000184937 | ALLPAVPSL | 2523 | HLA-B*55:01 |
| 5646 | WT1 | ENSG00000184937 | ALLPAVPSL | 2523 | HLA-B*58:01 |
| 5647 | WT1 | ENSG00000184937 | ALLPAVPSL | 2523 | HLA-C*01:02 |
| 5648 | WT1 | ENSG00000184937 | ALLPAVPSL | 2523 | HLA-C*02:02 |
| 5649 | WT1 | ENSG00000184937 | ALLPAVPSL | 2523 | HLA-C*03:03 |
| 5650 | WT1 | ENSG00000184937 | ALLPAVPSL | 2523 | HLA-C*03:04 |
| 5651 | WT1 | ENSG00000184937 | ALLPAVPSL | 2523 | HLA-C*07:04 |
| 5652 | WT1 | ENSG00000184937 | ALLPAVPSL | 2523 | HLA-C*14:02 |
| 5653 | WT1 | ENSG00000184937 | ALPVSGAAQW | 2524 | HLA-A*25:01 |
| 5654 | WT1 | ENSG00000184937 | ALPVSGAAQ | 2525 | HLA-C*01:02 |
| 5655 | WT1 | ENSG00000184937 | APPGASAYGSL | 2526 | HLA-B*07:02 |
| 5656 | WT1 | ENSG00000184937 | APPGASAYGSL | 2526 | HLA-C*01:02 |
| 5657 | WT1 | ENSG00000184937 | APPGASAYGSL | 2526 | HLA-C*07:02 |
| 5658 | WT1 | ENSG00000184937 | APPPAPPPPP | 2527 | HLA-C*04:01 |
| 5659 | WT1 | ENSG00000184937 | APPPAPPPP | 2528 | HLA-C*04:01 |
| 5660 | WT1 | ENSG00000184937 | APPPPPPPPP | 2529 | HLA-C*04:01 |
| 5661 | WT1 | ENSG00000184937 | APPPPPPPP | 2530 | HLA-B*56:01 |
| 5662 | WT1 | ENSG00000184937 | APPPPPPPP | 2530 | HLA-C*04:01 |
| 5663 | WT1 | ENSG00000184937 | APTLVRSASET | 2531 | HLA-B*07:02 |
| 5664 | WT1 | ENSG00000184937 | APTLVRSAS | 2532 | HLA-B*56:01 |
| 5665 | WT1 | ENSG00000184937 | APTLVRSA | 2533 | HLA-B*07:02 |
| 5666 | WT1 | ENSG00000184937 | APTLVRSA | 2533 | HLA-B*54:01 |
| 5667 | WT1 | ENSG00000184937 | APTLVRSA | 2533 | HLA-B*55:01 |
| 5668 | WT1 | ENSG00000184937 | APTLVRSA | 2533 | HLA-B*56:01 |
| 5669 | WT1 | ENSG00000184937 | APVLDFAPPGA | 2534 | HLA-B*56:01 |
| 5670 | WT1 | ENSG00000184937 | APVLDFAPPG | 2535 | HLA-B*56:01 |
| 5671 | WT1 | ENSG00000184937 | APVLDFAPP | 2536 | HLA-B*56:01 |
| 5672 | WT1 | ENSG00000184937 | APYLPSCL | 2537 | HLA-B*07:02 |
| 5673 | WT1 | ENSG00000184937 | APYLPSCL | 2537 | HLA-B*08:01 |
| 5674 | WT1 | ENSG00000184937 | APYLPSCL | 2537 | HLA-B*56:01 |
| 5675 | WT1 | ENSG00000184937 | AQFPNHSFK | 2538 | HLA-A*03:01 |
| 5676 | WT1 | ENSG00000184937 | AQFPNHSFK | 2538 | HLA-A*03:02 |
| 5677 | WT1 | ENSG00000184937 | AQFPNHSFK | 2538 | HLA-A*11:01 |
| 5678 | WT1 | ENSG00000184937 | AQFPNHSFK | 2538 | HLA-A*31:01 |
| 5679 | WT1 | ENSG00000184937 | AQFPNHSFK | 2538 | HLA-B*27:05 |
| 5680 | WT1 | ENSG00000184937 | AQFPNHSF | 2539 | HLA-A*23:01 |
| 5681 | WT1 | ENSG00000184937 | AQFPNHSF | 2539 | HLA-A*30:02 |
| 5682 | WT1 | ENSG00000184937 | AQFPNHSF | 2539 | HLA-A*32:01 |
| 5683 | WT1 | ENSG00000184937 | AQFPNHSF | 2539 | HLA-B*13:02 |
| 5684 | WT1 | ENSG00000184937 | AQFPNHSF | 2539 | HLA-B*15:01 |
| 5685 | WT1 | ENSG00000184937 | AQFPNHSF | 2539 | HLA-B*15:03 |
| 5686 | WT1 | ENSG00000184937 | AQFPNHSF | 2539 | HLA-B*27:05 |
| 5687 | WT1 | ENSG00000184937 | AQFPNHSF | 2539 | HLA-B*37:01 |
| 5688 | WT1 | ENSG00000184937 | AQFPNHSF | 2539 | HLA-B*39:01 |
| 5689 | WT1 | ENSG00000184937 | AQFPNHSF | 2539 | HLA-B*44:02 |
| 5690 | WT1 | ENSG00000184937 | AQFPNHSF | 2539 | HLA-B*46:01 |
| 5691 | WT1 | ENSG00000184937 | AQFPNHSF | 2539 | HLA-B*57:01 |
| 5692 | WT1 | ENSG00000184937 | AQFPNHSF | 2539 | HLA-B*58:01 |
| 5693 | WT1 | ENSG00000184937 | AQFPNHSF | 2539 | HLA-C*02:02 |
| 5694 | WT1 | ENSG00000184937 | AQFPNHSF | 2539 | HLA-C*14:02 |
| 5695 | WT1 | ENSG00000184937 | AQFPNHSF | 2539 | HLA-C*16:04 |
| 5696 | WT1 | ENSG00000184937 | AQWAPVLDF | 2540 | HLA-B*13:02 |
| 5697 | WT1 | ENSG00000184937 | AQWAPVLDF | 2540 | HLA-B*15:01 |
| 5698 | WT1 | ENSG00000184937 | AQWAPVLDF | 2540 | HLA-B*15:03 |
| 5699 | WT1 | ENSG00000184937 | ARSDELVREI | 2541 | HLA-B*27:05 |
| 5700 | WT1 | ENSG00000184937 | ASETSEKRPF | 2542 | HLA-C*16:01 |
| 5701 | WT1 | ENSG00000184937 | ASSGQARMF | 2543 | HLA-A*32:01 |
| 5702 | WT1 | ENSG00000184937 | ASSGQARMF | 2543 | HLA-B*58:01 |
| 5703 | WT1 | ENSG00000184937 | ASSGQARMF | 2543 | HLA-C*02:02 |
| 5704 | WT1 | ENSG00000184937 | ASSGQARMF | 2543 | HLA-C*16:01 |
| 5705 | WT1 | ENSG00000184937 | ASSGQARMF | 2543 | HLA-C*16:02 |
| 5706 | WT1 | ENSG00000184937 | CALPVSGAAQW | 2544 | HLA-B*57:01 |
| 5707 | WT1 | ENSG00000184937 | CALPVSGAAQW | 2544 | HLA-B*58:01 |
| 5708 | WT1 | ENSG00000184937 | CALPVSGAA | 2545 | HLA-B*56:01 |
| 5709 | WT1 | ENSG00000184937 | DELVRHHNM | 2546 | HLA-B*08:01 |
| 5710 | WT1 | ENSG00000184937 | DELVRHHNM | 2546 | HLA-B*37:01 |
| 5711 | WT1 | ENSG00000184937 | DFAPPGASAY | 2547 | HLA-A*25:01 |
| 5712 | WT1 | ENSG00000184937 | DFAPPGASAY | 2547 | HLA-A*26:01 |
| 5713 | WT1 | ENSG00000184937 | DFAPPGASAY | 2547 | HLA-A*29:02 |
| 5714 | WT1 | ENSG00000184937 | DFAPPGASAY | 2547 | HLA-A*30:02 |
| 5715 | WT1 | ENSG00000184937 | DFAPPGASAY | 2547 | HLA-C*14:02 |
| 5716 | WT1 | ENSG00000184937 | DFAPPGASA | 2548 | HLA-C*14:02 |
| 5717 | WT1 | ENSG00000184937 | DHLKTHTR | 2549 | HLA-A*33:01 |
| 5718 | WT1 | ENSG00000184937 | DPMGQQGSL | 2550 | HLA-B*07:02 |
| 5719 | WT1 | ENSG00000184937 | DPMGQQGSL | 2550 | HLA-B*08:01 |
| 5720 | WT1 | ENSG00000184937 | DPMGQQGSL | 2550 | HLA-B*35:01 |
| 5721 | WT1 | ENSG00000184937 | DPMGQQGSL | 2550 | HLA-B*35:03 |
| 5722 | WT1 | ENSG00000184937 | DPMGQQGSL | 2550 | HLA-B*51:01 |
| 5723 | WT1 | ENSG00000184937 | DPMGQQGSL | 2550 | HLA-C*07:02 |
| 5724 | WT1 | ENSG00000184937 | DVRDLNALL | 2551 | HLA-A*25:01 |
| 5725 | WT1 | ENSG00000184937 | DVRDLNALL | 2551 | HLA-A*26:01 |
| 5726 | WT1 | ENSG00000184937 | DVRDLNALL | 2551 | HLA-A*33:01 |
| 5727 | WT1 | ENSG00000184937 | DVRDLNAL | 2552 | HLA-B*08:01 |
| 5728 | WT1 | ENSG00000184937 | DVRRVPGV | 2553 | HLA-B*51:01 |
| 5729 | WT1 | ENSG00000184937 | EEQCLSAF | 2554 | HLA-B*18:01 |
| 5730 | WT1 | ENSG00000184937 | EGQSNHSTGY | 2555 | HLA-A*26:01 |
| 5731 | WT1 | ENSG00000184937 | EGQSNHSTGY | 2555 | HLA-A*30:02 |
| 5732 | WT1 | ENSG00000184937 | EGQSNHSTGY | 2555 | HLA-B*44:02 |
| 5733 | WT1 | ENSG00000184937 | EGQSNHSTGY | 2555 | HLA-B*44:03 |
| 5734 | WT1 | ENSG00000184937 | EGQSNHSTGY | 2555 | HLA-C*04:01 |
| 5735 | WT1 | ENSG00000184937 | EGQSNHSTGY | 2555 | HLA-C*07:01 |
| 5736 | WT1 | ENSG00000184937 | EQQYSVPPPVY | 2556 | HLA-A*30:02 |
| 5737 | WT1 | ENSG00000184937 | ESQPAIRNQGY | 2557 | HLA-A*01:01 |
| 5738 | WT1 | ENSG00000184937 | ESQPAIRNQGY | 2557 | HLA-A*25:01 |
| 5739 | WT1 | ENSG00000184937 | ESQPAIRNQGY | 2557 | HLA-A*26:01 |
| 5740 | WT1 | ENSG00000184937 | ESQPAIRNQGY | 2557 | HLA-A*30:02 |
| 5741 | WT1 | ENSG00000184937 | ESQPAIRNQGY | 2557 | HLA-B*44:02 |
| 5742 | WT1 | ENSG00000184937 | ETSEKRPFM | 2558 | HLA-A*26:01 |
| 5743 | WT1 | ENSG00000184937 | FAPPGASAY | 2559 | HLA-A*01:01 |
| 5744 | WT1 | ENSG00000184937 | FAPPGASAY | 2559 | HLA-A*25:01 |
| 5745 | WT1 | ENSG00000184937 | FAPPGASAY | 2559 | HLA-A*26:01 |
| 5746 | WT1 | ENSG00000184937 | FAPPGASAY | 2559 | HLA-A*29:02 |
| 5747 | WT1 | ENSG00000184937 | FAPPGASAY | 2559 | HLA-A*30:02 |
| 5748 | WT1 | ENSG00000184937 | FAPPGASAY | 2559 | HLA-A*32:01 |
| 5749 | WT1 | ENSG00000184937 | FAPPGASAY | 2559 | HLA-B*15:01 |
| 5750 | WT1 | ENSG00000184937 | FAPPGASAY | 2559 | HLA-B*15:03 |
| 5751 | WT1 | ENSG00000184937 | FAPPGASAY | 2559 | HLA-B*35:01 |
| 5752 | WT1 | ENSG00000184937 | FAPPGASAY | 2559 | HLA-B*39:01 |
| 5753 | WT1 | ENSG00000184937 | FAPPGASAY | 2559 | HLA-B*46:01 |
| 5754 | WT1 | ENSG00000184937 | FAPPGASAY | 2559 | HLA-B*51:01 |
| 5755 | WT1 | ENSG00000184937 | FAPPGASAY | 2559 | HLA-B*55:01 |
| 5756 | WT1 | ENSG00000184937 | FAPPGASAY | 2559 | HLA-B*58:01 |
| 5757 | WT1 | ENSG00000184937 | FAPPGASAY | 2559 | HLA-C*01:02 |
| 5758 | WT1 | ENSG00000184937 | FAPPGASAY | 2559 | HLA-C*02:02 |
| 5759 | WT1 | ENSG00000184937 | FAPPGASAY | 2559 | HLA-C*03:03 |
| 5760 | WT1 | ENSG00000184937 | FAPPGASAY | 2559 | HLA-C*03:04 |
| 5761 | WT1 | ENSG00000184937 | FAPPGASAY | 2559 | HLA-C*05:01 |
| 5762 | WT1 | ENSG00000184937 | FAPPGASAY | 2559 | HLA-C*07:04 |
| 5763 | WT1 | ENSG00000184937 | FAPPGASAY | 2559 | HLA-C*12:03 |
| 5764 | WT1 | ENSG00000184937 | FAPPGASAY | 2559 | HLA-C*14:02 |
| 5765 | WT1 | ENSG00000184937 | FAPPGASAY | 2559 | HLA-C*16:01 |
| 5766 | WT1 | ENSG00000184937 | FAPPGASAY | 2559 | HLA-C*16:02 |
| 5767 | WT1 | ENSG00000184937 | FAPPGASAY | 2559 | HLA-C*16:04 |
| 5768 | WT1 | ENSG00000184937 | FGPPPPSQA | 2560 | HLA-A*02:01 |
| 5769 | WT1 | ENSG00000184937 | FGPPPPSQA | 2560 | HLA-A*02:07 |
| 5770 | WT1 | ENSG00000184937 | FPNAPYLPSCL | 2561 | HLA-B*35:03 |
| 5771 | WT1 | ENSG00000184937 | FPNAPYLPSC | 2562 | HLA-B*54:01 |
| 5772 | WT1 | ENSG00000184937 | FPNAPYLPSC | 2562 | HLA-B*56:01 |
| 5773 | WT1 | ENSG00000184937 | FPNAPYLPS | 2563 | HLA-B*54:01 |
| 5774 | WT1 | ENSG00000184937 | FPNAPYLP | 2564 | HLA-B*54:01 |
| 5775 | WT1 | ENSG00000184937 | FTGTAGACRY | 2565 | HLA-A*01:01 |
| 5776 | WT1 | ENSG00000184937 | FTGTAGACRY | 2565 | HLA-A*29:02 |
| 5777 | WT1 | ENSG00000184937 | FTGTAGACRY | 2565 | HLA-A*30:02 |
| 5778 | WT1 | ENSG00000184937 | FTGTAGACR | 2566 | HLA-A*68:01 |
| 5779 | WT1 | ENSG00000184937 | FTGTAGACR | 2566 | HLA-C*07:06 |
| 5780 | WT1 | ENSG00000184937 | FTGTAGAC | 2567 | HLA-C*12:03 |
| 5781 | WT1 | ENSG00000184937 | FTVHFSGQF | 2568 | HLA-A*25:01 |
| 5782 | WT1 | ENSG00000184937 | FTVHFSGQF | 2568 | HLA-A*26:01 |
| 5783 | WT1 | ENSG00000184937 | FTVHFSGQF | 2568 | HLA-A*29:02 |
| 5784 | WT1 | ENSG00000184937 | FTVHFSGQF | 2568 | HLA-B*46:01 |
| 5785 | WT1 | ENSG00000184937 | FTVHFSGQF | 2568 | HLA-B*57:01 |
| 5786 | WT1 | ENSG00000184937 | FTVHFSGQF | 2568 | HLA-C*02:02 |
| 5787 | WT1 | ENSG00000184937 | GAAQWAPVL | 2569 | HLA-C*03:03 |
| 5788 | WT1 | ENSG00000184937 | GAAQWAPVL | 2569 | HLA-C*03:04 |
| 5789 | WT1 | ENSG00000184937 | GATLKGVAA | 2570 | HLA-B*54:01 |
| 5790 | WT1 | ENSG00000184937 | GATLKGVAA | 2570 | HLA-B*56:01 |
| 5791 | WT1 | ENSG00000184937 | GATLKGVAA | 2570 | HLA-C*03:04 |
| 5792 | WT1 | ENSG00000184937 | GATLKGVAA | 2570 | HLA-C*16:01 |
| 5793 | WT1 | ENSG00000184937 | GATLKGVA | 2571 | HLA-C*16:01 |
| 5794 | WT1 | ENSG00000184937 | GEKPYQCDF | 2572 | HLA-A*30:01 |
| 5795 | WT1 | ENSG00000184937 | GEKPYQCDF | 2572 | HLA-B*37:01 |
| 5796 | WT1 | ENSG00000184937 | GEKPYQCDF | 2572 | HLA-B*44:02 |
| 5797 | WT1 | ENSG00000184937 | GEKPYQCDF | 2572 | HLA-B*44:03 |
| 5798 | WT1 | ENSG00000184937 | GEKPYQCDF | 2572 | HLA-C*16:04 |
| 5799 | WT1 | ENSG00000184937 | GPAPPPAPP | 2573 | HLA-B*56:01 |
| 5800 | WT1 | ENSG00000184937 | GPFGPPPPSQA | 2574 | HLA-B*54:01 |
| 5801 | WT1 | ENSG00000184937 | GPFGPPPPSQA | 2574 | HLA-B*55:01 |
| 5802 | WT1 | ENSG00000184937 | GPFGPPPPSQA | 2574 | HLA-B*56:01 |
| 5803 | WT1 | ENSG00000184937 | GPFGPPPPSQ | 2575 | HLA-B*54:01 |
| 5804 | WT1 | ENSG00000184937 | GPFGPPPPS | 2576 | HLA-B*54:01 |
| 5805 | WT1 | ENSG00000184937 | GPFGPPPPS | 2576 | HLA-B*56:01 |
| 5806 | WT1 | ENSG00000184937 | GPFGPPPP | 2577 | HLA-B*56:01 |
| 5807 | WT1 | ENSG00000184937 | GQARMFPNAPY | 2578 | HLA-A*30:02 |
| 5808 | WT1 | ENSG00000184937 | GQARMFPNAPY | 2578 | HLA-B*15:01 |
| 5809 | WT1 | ENSG00000184937 | GQFTGTAGACR | 2579 | HLA-A*31:01 |
| 5810 | WT1 | ENSG00000184937 | GQFTGTAGACR | 2579 | HLA-B*13:02 |
| 5811 | WT1 | ENSG00000184937 | GQFTGTAGACR | 2579 | HLA-B*27:05 |
| 5812 | WT1 | ENSG00000184937 | GQFTGTAGACR | 2579 | HLA-C*07:06 |
| 5813 | WT1 | ENSG00000184937 | GQFTGTAGAC | 2580 | HLA-B*13:02 |
| 5814 | WT1 | ENSG00000184937 | GQFTGTAGAC | 2580 | HLA-B*15:01 |
| 5815 | WT1 | ENSG00000184937 | GQFTGTAGAC | 2580 | HLA-B*15:03 |
| 5816 | WT1 | ENSG00000184937 | GQFTGTAGAC | 2580 | HLA-B*27:05 |
| 5817 | WT1 | ENSG00000184937 | GQFTGTAGAC | 2580 | HLA-C*16:04 |
| 5818 | WT1 | ENSG00000184937 | GQFTGTAGA | 2581 | HLA-A*02:01 |
| 5819 | WT1 | ENSG00000184937 | GQFTGTAGA | 2581 | HLA-A*02:03 |
| 5820 | WT1 | ENSG00000184937 | GQFTGTAGA | 2581 | HLA-A*32:01 |
| 5821 | WT1 | ENSG00000184937 | GQFTGTAGA | 2581 | HLA-B*13:02 |
| 5822 | WT1 | ENSG00000184937 | GQFTGTAGA | 2581 | HLA-B*15:01 |
| 5823 | WT1 | ENSG00000184937 | GQFTGTAGA | 2581 | HLA-B*15:03 |
| 5824 | WT1 | ENSG00000184937 | GQFTGTAGA | 2581 | HLA-B*27:05 |
| 5825 | WT1 | ENSG00000184937 | GQQGSLGEQQY | 2582 | HLA-A*30:02 |
| 5826 | WT1 | ENSG00000184937 | GQQGSLGEQQY | 2582 | HLA-B*15:01 |
| 5827 | WT1 | ENSG00000184937 | GQQGSLGEQQY | 2582 | HLA-B*15:03 |
| 5828 | WT1 | ENSG00000184937 | GQQGSLGEQQY | 2582 | HLA-B*27:05 |
| 5829 | WT1 | ENSG00000184937 | GQQGSLGEQQY | 2582 | HLA-B*39:01 |
| 5830 | WT1 | ENSG00000184937 | GQQGSLGEQQY | 2582 | HLA-C*07:04 |
| 5831 | WT1 | ENSG00000184937 | GQQGSLGEQQY | 2582 | HLA-C*16:04 |
| 5832 | WT1 | ENSG00000184937 | GQSNHSTGY | 2583 | HLA-A*26:01 |
| 5833 | WT1 | ENSG00000184937 | GQSNHSTGY | 2583 | HLA-A*29:02 |
| 5834 | WT1 | ENSG00000184937 | GQSNHSTGY | 2583 | HLA-A*30:02 |
| 5835 | WT1 | ENSG00000184937 | GQSNHSTGY | 2583 | HLA-A*32:01 |
| 5836 | WT1 | ENSG00000184937 | GQSNHSTGY | 2583 | HLA-B*13:02 |
| 5837 | WT1 | ENSG00000184937 | GQSNHSTGY | 2583 | HLA-B*15:01 |
| 5838 | WT1 | ENSG00000184937 | GQSNHSTGY | 2583 | HLA-B*15:03 |
| 5839 | WT1 | ENSG00000184937 | GQSNHSTGY | 2583 | HLA-B*27:02 |
| 5840 | WT1 | ENSG00000184937 | GQSNHSTGY | 2583 | HLA-B*27:05 |
| 5841 | WT1 | ENSG00000184937 | GQSNHSTGY | 2583 | HLA-B*39:01 |
| 5842 | WT1 | ENSG00000184937 | GQSNHSTGY | 2583 | HLA-B*44:03 |
| 5843 | WT1 | ENSG00000184937 | GQSNHSTGY | 2583 | HLA-B*46:01 |
| 5844 | WT1 | ENSG00000184937 | GQSNHSTGY | 2583 | HLA-C*02:02 |
| 5845 | WT1 | ENSG00000184937 | GQSNHSTGY | 2583 | HLA-C*07:04 |
| 5846 | WT1 | ENSG00000184937 | GQSNHSTGY | 2583 | HLA-C*16:04 |
| 5847 | WT1 | ENSG00000184937 | GSLGGPAPPP | 2584 | HLA-A*11:01 |
| 5848 | WT1 | ENSG00000184937 | GSLGGPAPPP | 2584 | HLA-C*06:02 |
| 5849 | WT1 | ENSG00000184937 | GSLGGPAPPP | 2584 | HLA-C*07:02 |
| 5850 | WTI | ENSG00000184937 | GSQALLLRTPY | 2585 | HLA-A*30:02 |
| 5851 | WT1 | ENSG00000184937 | GSSSSVKW | 2586 | HLA-A*32:01 |
| 5852 | WT1 | ENSG00000184937 | GSSSSVKW | 2586 | HLA-B*57:01 |
| 5853 | WT1 | ENSG00000184937 | GSSSSVKW | 2586 | HLA-B*58:01 |
| 5854 | WT1 | ENSG00000184937 | GTAGACRY | 2587 | HLA-A*30:02 |
| 5855 | WT1 | ENSG00000184937 | GVAAGSSSSVK | 2588 | HLA-A*03:01 |
| 5856 | WT1 | ENSG00000184937 | GVAAGSSSSVK | 2588 | HLA-A*03:02 |
| 5857 | WT1 | ENSG00000184937 | GVAAGSSSSVK | 2588 | HLA-A*11:01 |
| 5858 | WT1 | ENSG00000184937 | GVAAGSSSSV | 2589 | HLA-A*02:03 |
| 5859 | WT1 | ENSG00000184937 | GVAAGSSSSV | 2589 | HLA-B*13:02 |
| 5860 | WT1 | ENSG00000184937 | GVAPTLVRSA | 2590 | HLA-B*56:01 |
| 5861 | WT1 | ENSG00000184937 | GVFRGIQDV | 2591 | HLA-A*02:01 |
| 5862 | WT1 | ENSG00000184937 | GVFRGIQDV | 2591 | HLA-A*02:03 |
| 5863 | WT1 | ENSG00000184937 | GVFRGIQDV | 2591 | HLA-A*02:04 |
| 5864 | WT1 | ENSG00000184937 | GVFRGIQDV | 2591 | HLA-B*13:02 |
| 5865 | WTI | ENSG00000184937 | HAAQFPNHSF | 2592 | HLA-A*25:01 |
| 5866 | WT1 | ENSG00000184937 | HAAQFPNHSF | 2592 | HLA-A*26:01 |
| 5867 | WT1 | ENSG00000184937 | HAAQFPNHSF | 2592 | HLA-A*32:01 |
| 5868 | WT1 | ENSG00000184937 | HAAQFPNHSF | 2592 | HLA-A*33:01 |
| 5869 | WT1 | ENSG00000184937 | HAAQFPNHSF | 2592 | HLA-A*68:02 |
| 5870 | WT1 | ENSG00000184937 | HAAQFPNHSF | 2592 | HLA-B*35:01 |
| 5871 | WT1 | ENSG00000184937 | HAAQFPNHSF | 2592 | HLA-B*35:03 |
| 5872 | WT1 | ENSG00000184937 | HAAQFPNHSF | 2592 | HLA-B*46:01 |
| 5873 | WT1 | ENSG00000184937 | HAAQFPNHSF | 2592 | HLA-B*54:01 |
| 5874 | WT1 | ENSG00000184937 | HAAQFPNHSF | 2592 | HLA-B*57:01 |
| 5875 | WT1 | ENSG00000184937 | HAAQFPNHSF | 2592 | HLA-B*58:01 |
| 5876 | WT1 | ENSG00000184937 | HAAQFPNHSF | 2592 | HLA-C*02:02 |
| 5877 | WT1 | ENSG00000184937 | HAAQFPNHSF | 2592 | HLA-C*03:03 |
| 5878 | WT1 | ENSG00000184937 | HAAQFPNHSF | 2592 | HLA-C*03:04 |
| 5879 | WT1 | ENSG00000184937 | HAAQFPNHSF | 2592 | HLA-C*07:06 |
| 5880 | WT1 | ENSG00000184937 | HAAQFPNHSF | 2592 | HLA-C*16:04 |
| 5881 | WT1 | ENSG00000184937 | HEDPMGQQGSL | 2593 | HLA-A*30:01 |
| 5882 | WT1 | ENSG00000184937 | HEDPMGQQGSL | 2593 | HLA-B*39:01 |
| 5883 | WT1 | ENSG00000184937 | HEDPMGQQGSL | 2593 | HLA-B*40:01 |
| 5884 | WT1 | ENSG00000184937 | HEDPMGQQGSL | 2593 | HLA-B*49:01 |
| 5885 | WT1 | ENSG00000184937 | HEDPMGQQG | 2594 | HLA-B*49:01 |
| 5886 | WT1 | ENSG00000184937 | HEEQCLSAF | 2595 | HLA-B*18:01 |
| 5887 | WT1 | ENSG00000184937 | HHAAQFPNHSF | 2596 | HLA-B*38:01 |
| 5888 | WT1 | ENSG00000184937 | HQRNMTKL | 2597 | HLA-B*15:03 |
| 5889 | WT1 | ENSG00000184937 | HSFIKQEPSW | 2598 | HLA-B*57:01 |
| 5890 | WT1 | ENSG00000184937 | HTPSHHAAQF | 2599 | HLA-A*25:01 |
| 5891 | WT1 | ENSG00000184937 | HTPSHHAAQF | 2599 | HLA-A*26:01 |
| 5892 | WT1 | ENSG00000184937 | HTTPILCGA | 2600 | HLA-A*68:02 |
| 5893 | WT1 | ENSG00000184937 | IRNQGYSTVTF | 2601 | HLA-B*27:02 |
| 5894 | WT1 | ENSG00000184937 | IRNQGYSTVTF | 2601 | HLA-C*16:04 |
| 5895 | WT1 | ENSG00000184937 | LDFAPPGASAY | 2602 | HLA-A*30:02 |
| 5896 | WT1 | ENSG00000184937 | LDFAPPGASAY | 2602 | HLA-B*18:01 |
| 5897 | WT1 | ENSG00000184937 | LDFAPPGASAY | 2602 | HLA-C*16:04 |
| 5898 | WT1 | ENSG00000184937 | LGATLKGVAA | 2603 | HLA-B*54:01 |
| 5899 | WT1 | ENSG00000184937 | LGGGGGCAL | 2604 | HLA-C*03:04 |
| 5900 | WT1 | ENSG00000184937 | LLPAVPSLGG | 2605 | HLA-A*02:07 |
| 5901 | WT1 | ENSG00000184937 | LLPAVPSL | 2606 | HLA-A*02:07 |
| 5902 | WT1 | ENSG00000184937 | LLPAVPSL | 2606 | HLA-A*24:02 |
| 5903 | WT1 | ENSG00000184937 | LLPAVPSL | 2606 | HLA-B*51:01 |
| 5904 | WT1 | ENSG00000184937 | LLPAVPSL | 2606 | HLA-C*01:02 |
| 5905 | WT1 | ENSG00000184937 | LPAVPSLGG | 2607 | HLA-B*51:01 |
| 5906 | WT1 | ENSG00000184937 | LPAVPSLGG | 2607 | HLA-B*54:01 |
| 5907 | WT1 | ENSG00000184937 | LPAVPSLGG | 2607 | HLA-B*56:01 |
| 5908 | WT1 | ENSG00000184937 | LPVSGAAQW | 2608 | HLA-A*25:01 |
| 5909 | WT1 | ENSG00000184937 | LPVSGAAQW | 2608 | HLA-B*35:01 |
| 5910 | WT1 | ENSG00000184937 | LPVSGAAQW | 2608 | HLA-B*35:03 |
| 5911 | WT1 | ENSG00000184937 | LPVSGAAQW | 2608 | HLA-B*51:01 |
| 5912 | WT1 | ENSG00000184937 | LPVSGAAQW | 2608 | HLA-B*56:01 |
| 5913 | WT1 | ENSG00000184937 | LSHLQMHSR | 2609 | HLA-A*33:01 |
| 5914 | WT1 | ENSG00000184937 | LVRSASETSEK | 2610 | HLA-A*03:01 |
| 5915 | WT1 | ENSG00000184937 | LYQMTSQL | 2611 | HLA-C*14:02 |
| 5916 | WT1 | ENSG00000184937 | MGSDVRDL | 2612 | HLA-C*16:01 |
| 5917 | WT1 | ENSG00000184937 | MHQRNMTKL | 2613 | HLA-B*38:01 |
| 5918 | WT1 | ENSG00000184937 | MTKLQLAL | 2614 | HLA-B*08:01 |
| 5919 | WT1 | ENSG00000184937 | MTKLQLAL | 2614 | HLA-C*03:04 |
| 5920 | WT1 | ENSG00000184937 | MTKLQLAL | 2614 | HLA-C*07:01 |
| 5921 | WT1 | ENSG00000184937 | MTSQLECMTVV | 2615 | HLA-A*25:01 |
| 5922 | WT1 | ENSG00000184937 | MTSQLECMTVV | 2615 | HLA-B*57:01 |
| 5923 | WT1 | ENSG00000184937 | MTSQLECMTVV | 2615 | HLA-B*58:01 |
| 5924 | WT1 | ENSG00000184937 | NAPYLPSCL | 2616 | HLA-C*01:02 |
| 5925 | WT1 | ENSG00000184937 | NAPYLPSC | 2617 | HLA-B*51:01 |
| 5926 | WT1 | ENSG00000184937 | NLGATLKGV | 2618 | HLA-A*02:03 |
| 5927 | WT1 | ENSG00000184937 | NLYQMTSQL | 2619 | HLA-A*02:01 |
| 5928 | WT1 | ENSG00000184937 | NLYQMTSQL | 2619 | HLA-A*02:03 |
| 5929 | WT1 | ENSG00000184937 | NLYQMTSQL | 2619 | HLA-C*01:02 |
| 5930 | WT1 | ENSG00000184937 | NLYQMTSQL | 2619 | HLA-C*14:02 |
| 5931 | WT1 | ENSG00000184937 | NMTKLQLAL | 2620 | HLA-B*08:01 |
| 5932 | WT1 | ENSG00000184937 | NMTKLQLAL | 2620 | HLA-B*13:02 |
| 5933 | WT1 | ENSG00000184937 | NQGYSTVTF | 2621 | HLA-B*15:01 |
| 5934 | WT1 | ENSG00000184937 | NQGYSTVTF | 2621 | HLA-B*15:03 |
| 5935 | WT1 | ENSG00000184937 | NQGYSTVTF | 2621 | HLA-B*18:01 |
| 5936 | WT1 | ENSG00000184937 | NQGYSTVTF | 2621 | HLA-B*38:01 |
| 5937 | WT1 | ENSG00000184937 | NQGYSTVTF | 2621 | HLA-B*39:01 |
| 5938 | WT1 | ENSG00000184937 | NQGYSTVTF | 2621 | HLA-C*07:04 |
| 5939 | WT1 | ENSG00000184937 | NQGYSTVTF | 2621 | HLA-C*14:02 |
| 5940 | WT1 | ENSG00000184937 | NQMNLGATLK | 2622 | HLA-A*11:01 |
| 5941 | WT1 | ENSG00000184937 | NQMNLGATL | 2623 | HLA-A*23:01 |
| 5942 | WT1 | ENSG00000184937 | NQMNLGATL | 2623 | HLA-B*13:02 |
| 5943 | WT1 | ENSG00000184937 | NQMNLGATL | 2623 | HLA-B*15:01 |
| 5944 | WT1 | ENSG00000184937 | NQMNLGATL | 2623 | HLA-B*15:03 |
| 5945 | WT1 | ENSG00000184937 | NQMNLGATL | 2623 | HLA-B*27:05 |
| 5946 | WT1 | ENSG00000184937 | NQMNLGATL | 2623 | HLA-B*38:01 |
| 5947 | WT1 | ENSG00000184937 | NQMNLGATL | 2623 | HLA-B*39:01 |
| 5948 | WT1 | ENSG00000184937 | NQMNLGATL | 2623 | HLA-C*03:03 |
| 5949 | WT1 | ENSG00000184937 | NQMNLGATL | 2623 | HLA-C*07:02 |
| 5950 | WT1 | ENSG00000184937 | NQMNLGATL | 2623 | HLA-C*07:04 |
| 5951 | WT1 | ENSG00000184937 | NQMNLGATL | 2623 | HLA-C*14:02 |
| 5952 | WT1 | ENSG00000184937 | NQMNLGATL | 2623 | HLA-C*16:04 |
| 5953 | WT1 | ENSG00000184937 | PPPPPPPPP | 2624 | HLA-C*04:01 |
| 5954 | WT1 | ENSG00000184937 | QALLLRTPY | 2625 | HLA-A*01:01 |
| 5955 | WT1 | ENSG00000184937 | QALLLRTPY | 2625 | HLA-A*29:02 |
| 5956 | WT1 | ENSG00000184937 | QALLLRTPY | 2625 | HLA-A*30:02 |
| 5957 | WT1 | ENSG00000184937 | QALLLRTPY | 2625 | HLA-B*15:03 |
| 5958 | WT1 | ENSG00000184937 | QALLLRTPY | 2625 | HLA-B*35:01 |
| 5959 | WT1 | ENSG00000184937 | QALLLRTPY | 2625 | HLA-B*46:01 |
| 5960 | WT1 | ENSG00000184937 | QALLLRTPY | 2625 | HLA-B*57:01 |
| 5961 | WT1 | ENSG00000184937 | QALLLRTPY | 2625 | HLA-C*02:02 |
| 5962 | WT1 | ENSG00000184937 | QALLLRTPY | 2625 | HLA-C*07:01 |
| 5963 | WT1 | ENSG00000184937 | QALLLRTPY | 2625 | HLA-C*07:04 |
| 5964 | WT1 | ENSG00000184937 | QALLLRTPY | 2625 | HLA-C*16:01 |
| 5965 | WT1 | ENSG00000184937 | QASSGQARMF | 2626 | HLA-C*16:01 |
| 5966 | WT1 | ENSG00000184937 | QASSGQARMF | 2626 | HLA-C*16:02 |
| 5967 | WT1 | ENSG00000184937 | QASSGQARM | 2627 | HLA-C*16:01 |
| 5968 | WT1 | ENSG00000184937 | QDVRRVPGV | 2628 | HLA-B*37:01 |
| 5969 | WT1 | ENSG00000184937 | QFTGTAGACRY | 2629 | HLA-A*29:02 |
| 5970 | WT1 | ENSG00000184937 | QGYSTVTF | 2630 | HLA-B*08:01 |
| 5971 | WT1 | ENSG00000184937 | QGYSTVTF | 2630 | HLA-B*15:03 |
| 5972 | WT1 | ENSG00000184937 | QGYSTVTF | 2630 | HLA-B*18:01 |
| 5973 | WT1 | ENSG00000184937 | QGYSTVTF | 2630 | HLA-B*37:01 |
| 5974 | WT1 | ENSG00000184937 | QGYSTVTF | 2630 | HLA-B*46:01 |
| 5975 | WT1 | ENSG00000184937 | QGYSTVTF | 2630 | HLA-C*12:03 |
| 5976 | WT1 | ENSG00000184937 | QMNLGATLK | 2631 | HLA-A*03:01 |
| 5977 | WT1 | ENSG00000184937 | QMNLGATLK | 2631 | HLA-A*03:02 |
| 5978 | WTI | ENSG00000184937 | QMNLGATLK | 2631 | HLA-A*II:01 |
| 5979 | WT1 | ENSG00000184937 | QMNLGATL | 2632 | HLA-B*07:02 |
| 5980 | WT1 | ENSG00000184937 | QMNLGATL | 2632 | HLA-B*08:01 |
| 5981 | WT1 | ENSG00000184937 | QMNLGATL | 2632 | HLA-C*01:02 |
| 5982 | WT1 | ENSG00000184937 | QMNLGATL | 2632 | HLA-C*14:02 |
| 5982 | WT1 | ENSG00000184937 | QPAIRNQGY | 2633 | HLA-B*35:01 |
| 5984 | WT1 | ENSG00000184937 | QPAIRNQGY | 2633 | HLA-B*55:01 |
| 5985 | WT1 | ENSG00000184937 | QQGSLGEQQY | 2634 | HLA-A*30:02 |
| 5986 | WT1 | ENSG00000184937 | QQGSLGEQQY | 2634 | HLA-B*15:01 |
| 5987 | WT1 | ENSG00000184937 | QQGSLGEQQY | 2634 | HLA-C*07:04 |
| 5988 | WT1 | ENSG00000184937 | QQYSVPPPVY | 2635 | HLA-A*30:02 |
| 5989 | WT1 | ENSG00000184937 | QQYSVPPPVY | 2635 | HLA-A*32:01 |
| 5990 | WT1 | ENSG00000184937 | QQYSVPPPVY | 2635 | HLA-B*15:01 |
| 5991 | WT1 | ENSG00000184937 | QQYSVPPPVY | 2635 | HLA-B*15:03 |
| 5992 | WT1 | ENSG00000184937 | QQYSVPPPVY | 2635 | HLA-B*39:01 |
| 5993 | WTI | ENSG00000184937 | QQYSVPPPVY | 2635 | HLA-C*I6:04 |
| 5994 | WT1 | ENSG00000184937 | QQYSVPPPV | 2636 | HLA-A*02:01 |
| 5995 | WT1 | ENSG00000184937 | QQYSVPPPV | 2636 | HLA-A*02:03 |
| 5996 | WT1 | ENSG00000184937 | QQYSVPPPV | 2636 | HLA-A*02:07 |
| 5997 | WT1 | ENSG00000184937 | QQYSVPPPV | 2636 | HLA-A*30:01 |
| 5998 | WT1 | ENSG00000184937 | QQYSVPPPV | 2636 | HLA-A*32:01 |
| 5999 | WT1 | ENSG00000184937 | QQYSVPPPV | 2636 | HLA-B*13:02 |
| 6000 | WT1 | ENSG00000184937 | QQYSVPPPV | 2636 | HLA-B*37:01 |
| 6001 | WT1 | ENSG00000184937 | QQYSVPPPV | 2636 | HLA-B*49:01 |
| 6002 | WT1 | ENSG00000184937 | QQYSVPPPV | 2636 | HLA-B*55:01 |
| 6003 | WT1 | ENSG00000184937 | QQYSVPPPV | 2636 | HLA-B*56:01 |
| 6004 | WT1 | ENSG00000184937 | QQYSVPPPV | 2636 | HLA-C*02:02 |
| 6005 | WT1 | ENSG00000184937 | QQYSVPPPV | 2636 | HLA-C*06:02 |
| 6006 | WT1 | ENSG00000184937 | QRNMTKLQL | 2637 | HLA-C*06:02 |
| 6007 | WT1 | ENSG00000184937 | QSNHSTGY | 2638 | HLA-A*30:02 |
| 6008 | WT1 | ENSG00000184937 | QSNHSTGY | 2638 | HLA-A*32:01 |
| 6009 | WT1 | ENSG00000184937 | QYSVPPPVY | 2639 | HLA-A*23:01 |
| 6010 | WT1 | ENSG00000184937 | QYSVPPPVY | 2639 | HLA-A*24:02 |
| 6011 | WT1 | ENSG00000184937 | QYSVPPPVY | 2639 | HLA-A*29:02 |
| 6012 | WT1 | ENSG00000184937 | QYSVPPPVY | 2639 | HLA-A*30:02 |
| 6013 | WT1 | ENSG00000184937 | QYSVPPPVY | 2639 | HLA-A*32:01 |
| 6014 | WT1 | ENSG00000184937 | QYSVPPPVY | 2639 | HLA-B*15:03 |
| 6015 | WT1 | ENSG00000184937 | QYSVPPPVY | 2639 | HLA-C*01:02 |
| 6016 | WT1 | ENSG00000184937 | QYSVPPPVY | 2639 | HLA-C*14:02 |
| 6017 | WT1 | ENSG00000184937 | QYSVPPPVY | 2639 | HLA-C*16:01 |
| 6018 | WT1 | ENSG00000184937 | RIHTHGVFR | 2640 | HLA-A*03:01 |
| 6019 | WT1 | ENSG00000184937 | RIHTHGVFR | 2640 | HLA-A*31:01 |
| 6020 | WT1 | ENSG00000184937 | RMFPNAPYL | 2641 | HLA-A*02:01 |
| 6021 | WT1 | ENSG00000184937 | RMFPNAPYL | 2641 | HLA-A*02:04 |
| 6022 | WT1 | ENSG00000184937 | RMFPNAPYL | 2641 | HLA-A*02:07 |
| 6023 | WT1 | ENSG00000184937 | RMFPNAPYL | 2641 | HLA-A*03:01 |
| 6024 | WT1 | ENSG00000184937 | RMFPNAPYL | 2641 | HLA-A*23:01 |
| 6025 | WT1 | ENSG00000184937 | RMFPNAPYL | 2641 | HLA-A*24:02 |
| 6026 | WT1 | ENSG00000184937 | RMFPNAPYL | 2641 | HLA-A*32:01 |
| 6027 | WT1 | ENSG00000184937 | RMFPNAPYL | 2641 | HLA-B*57:01 |
| 6028 | WT1 | ENSG00000184937 | RMFPNAPYL | 2641 | HLA-C*02:02 |
| 6029 | WT1 | ENSG00000184937 | RMFPNAPY | 2642 | HLA-A*30:02 |
| 6030 | WT1 | ENSG00000184937 | RNMTKLQL | 2643 | HLA-B*08:01 |
| 6031 | WT1 | ENSG00000184937 | RRVPGVAPTL | 2644 | HLA-B*27:05 |
| 6032 | WT1 | ENSG00000184937 | RRVPGVAPTL | 2644 | HLA-C*16:04 |
| 6033 | WT1 | ENSG00000184937 | RTPYSSDNLY | 2645 | HLA-A*01:01 |
| 6034 | WT1 | ENSG00000184937 | RTPYSSDNL | 2646 | HLA-C*01:02 |
| 6035 | WT1 | ENSG00000184937 | RVPGVAPTLVR | 2647 | HLA-A*03:01 |
| 6036 | WT1 | ENSG00000184937 | RVPGVAPTLVR | 2647 | HLA-A*31:01 |
| 6037 | WT1 | ENSG00000184937 | RVPGVAPTLV | 2648 | HLA-A*02:01 |
| 6038 | WT1 | ENSG00000184937 | RVPGVAPTLV | 2648 | HLA-A*02:03 |
| 6039 | WT1 | ENSG00000184937 | RVPGVAPTLV | 2648 | HLA-A*02:07 |
| 6040 | WT1 | ENSG00000184937 | RVPGVAPTL | 2649 | HLA-A*02:04 |
| 6041 | WT1 | ENSG00000184937 | RVPGVAPTL | 2649 | HLA-A*02:07 |
| 6042 | WT1 | ENSG00000184937 | RVPGVAPTL | 2649 | HLA-A*23:01 |
| 6043 | WT1 | ENSG00000184937 | RVPGVAPTL | 2649 | HLA-A*24:02 |
| 6044 | WT1 | ENSG00000184937 | RVPGVAPTL | 2649 | HLA-A*25:01 |
| 6045 | WT1 | ENSG00000184937 | RVPGVAPTL | 2649 | HLA-A*32:01 |
| 6046 | WT1 | ENSG00000184937 | RVPGVAPTL | 2649 | HLA-B*07:02 |
| 6047 | WT1 | ENSG00000184937 | RVPGVAPTL | 2649 | HLA-B*13:02 |
| 6048 | WT1 | ENSG00000184937 | RVPGVAPTL | 2649 | HLA-B*58:01 |
| 6049 | WT1 | ENSG00000184937 | RVPGVAPTL | 2649 | HLA-C*01:02 |
| 6050 | WT1 | ENSG00000184937 | RVPGVAPTL | 2649 | HLA-C*03:04 |
| 6051 | WT1 | ENSG00000184937 | RYFKLSHLQM | 2650 | HLA-A*24:02 |
| 6052 | WT1 | ENSG00000184937 | SASETSEKRPF | 2651 | HLA-C*16:01 |
| 6053 | WT1 | ENSG00000184937 | SASETSEKR | 2652 | HLA-A*68:01 |
| 6054 | WT1 | ENSG00000184937 | SASETSEKR | 2652 | HLA-C*07:06 |
| 6055 | WT1 | ENSG00000184937 | SDELVRHHNM | 2653 | HLA-B*37:01 |
| 6056 | WT1 | ENSG00000184937 | SDNHTTPIL | 2654 | HLA-B*37:01 |
| 6057 | WT1 | ENSG00000184937 | SDNHTTPI | 2655 | HLA-B*37:01 |
| 6058 | WT1 | ENSG00000184937 | SDVRDLNAL | 2656 | HLA-A*30:01 |
| 6059 | WT1 | ENSG00000184937 | SDVRDLNAL | 2656 | HLA-B*37:01 |
| 6060 | WT1 | ENSG00000184937 | SDVRDLNAL | 2656 | HLA-B*40:01 |
| 6061 | WT1 | ENSG00000184937 | SDVRDLNAL | 2656 | HLA-B*40:02 |
| 6062 | WT1 | ENSG00000184937 | SEKPFSCRW | 2657 | HLA-B*44:02 |
| 6063 | WT1 | ENSG00000184937 | SEKPFSCRW | 2657 | HLA-B*44:03 |
| 6064 | WT1 | ENSG00000184937 | SETSEKRPF | 2658 | HLA-B*37:01 |
| 6065 | WT1 | ENSG00000184937 | SETSEKRPF | 2658 | HLA-B*44:02 |
| 6066 | WT1 | ENSG00000184937 | SETSEKRPF | 2658 | HLA-B*44:03 |
| 6067 | WT1 | ENSG00000184937 | SETSEKRPF | 2658 | HLA-C*16:01 |
| 6068 | WT1 | ENSG00000184937 | SFIKQEPSW | 2659 | HLA-A*23:01 |
| 6069 | WT1 | ENSG00000184937 | SFIKQEPSW | 2659 | HLA-A*24:02 |
| 6070 | WT1 | ENSG00000184937 | SGQFTGTAGAC | 2660 | HLA-C*06:02 |
| 6071 | WT1 | ENSG00000184937 | SGQFTGTAGAC | 2660 | HLA-C*12:03 |
| 6072 | WT1 | ENSG00000184937 | SLGEQQYSV | 2661 | HLA-A*02:01 |
| 6073 | WT1 | ENSG00000184937 | SLGEQQYSV | 2661 | HLA-A*02:03 |
| 6074 | WT1 | ENSG00000184937 | SLGEQQYSV | 2661 | HLA-A*02:04 |
| 6075 | WT1 | ENSG00000184937 | SLGEQQYSV | 2661 | HLA-B*13:02 |
| 6076 | WT1 | ENSG00000184937 | SLGEQQYSV | 2661 | HLA-B*55:01 |
| 6077 | WT1 | ENSG00000184937 | SLGGPAPPP | 2662 | HLA-A*03:02 |
| 6078 | WT1 | ENSG00000184937 | SLGGPAPPP | 2662 | HLA-C*06:02 |
| 6079 | WT1 | ENSG00000184937 | SQALLLRTPY | 2663 | HLA-A*30:02 |
| 6080 | WT1 | ENSG00000184937 | SQALLLRTPY | 2663 | HLA-A*32:01 |
| 6081 | WT1 | ENSG00000184937 | SQALLLRTPY | 2663 | HLA-B*15:01 |
| 6082 | WT1 | ENSG00000184937 | SQALLLRTPY | 2663 | HLA-B*15:03 |
| 6083 | WT1 | ENSG00000184937 | SQALLLRTPY | 2663 | HLA-C*02:02 |
| 6084 | WT1 | ENSG00000184937 | SQALLLRTPY | 2663 | HLA-C*07:04 |
| 6085 | WT1 | ENSG00000184937 | SQPAIRNQGY | 2664 | HLA-A*26:01 |
| 6086 | WT1 | ENSG00000184937 | SQPAIRNQGY | 2664 | HLA-A*30:02 |
| 6087 | WT1 | ENSG00000184937 | SQPAIRNQGY | 2664 | HLA-A*32:01 |
| 6088 | WT1 | ENSG00000184937 | SQPAIRNQGY | 2664 | HLA-B*15:01 |
| 6089 | WT1 | ENSG00000184937 | SQPAIRNQGY | 2664 | HLA-B*15:03 |
| 6090 | WT1 | ENSG00000184937 | SQPAIRNQGY | 2664 | HLA-C*07:04 |
| 6091 | WT1 | ENSG00000184937 | SSDNLYQM | 2665 | HLA-C*05:01 |
| 6092 | WT1 | ENSG00000184937 | SSGQARMF | 2666 | HLA-C*16:01 |
| 6093 | WT1 | ENSG00000184937 | STVTFDGTPSY | 2667 | HLA-A*01:01 |
| 6094 | WT1 | ENSG00000184937 | STVTFDGTPSY | 2667 | HLA-A*25:01 |
| 6095 | WT1 | ENSG00000184937 | STVTFDGTPSY | 2667 | HLA-A*26:01 |
| 6096 | WT1 | ENSG00000184937 | STVTFDGTPSY | 2667 | HLA-A*29:02 |
| 6097 | WT1 | ENSG00000184937 | STVTFDGTPSY | 2667 | HLA-A*30:02 |
| 6098 | WT1 | ENSG00000184937 | STVTFDGTPSY | 2667 | HLA-B*46:01 |
| 6099 | WT1 | ENSG00000184937 | STVTFDGTPSY | 2667 | HLA-B*57:01 |
| 6100 | WT1 | ENSG00000184937 | STVTFDGTPSY | 2667 | HLA-B*58:01 |
| 6101 | WT1 | ENSG00000184937 | SVPPPVYGC | 2668 | HLA-A*02:01 |
| 6102 | WT1 | ENSG00000184937 | SVPPPVYGC | 2668 | HLA-A*02:07 |
| 6103 | WT1 | ENSG00000184937 | TEGQSNHSTGY | 2669 | HLA-B*27:02 |
| 6104 | WT1 | ENSG00000184937 | TEGQSNHSTGY | 2669 | HLA-B*44:02 |
| 6105 | WT1 | ENSG00000184937 | TEGQSNHSTGY | 2669 | HLA-B*44:03 |
| 6106 | WT1 | ENSG00000184937 | TEGQSNHSTGY | 2669 | HLA-C*04:01 |
| 6107 | WT1 | ENSG00000184937 | TEGQSNHSTGY | 2669 | HLA-C*16:04 |
| 6108 | WT1 | ENSG00000184937 | TFDGTPSYG | 2670 | HLA-C*04:01 |
| 6109 | WT1 | ENSG00000184937 | TFDGTPSY | 2671 | HLA-A*32:01 |
| 6110 | WT1 | ENSG00000184937 | TFDGTPSY | 2671 | HLA-B*18:01 |
| 6111 | WT1 | ENSG00000184937 | TFDGTPSY | 2671 | HLA-C*04:01 |
| 6112 | WT1 | ENSG00000184937 | TFDGTPSY | 2671 | HLA-C*14:02 |
| 6113 | WT1 | ENSG00000184937 | TGKTSEKPF | 2672 | HLA-C*16:01 |
| 6114 | WT1 | ENSG00000184937 | TGSQALLLR | 2673 | HLA-A*68:01 |
| 6115 | WT1 | ENSG00000184937 | TGSQALLLR | 2673 | HLA-C*07:06 |
| 6116 | WT1 | ENSG00000184937 | TGTAGACRY | 2674 | HLA-B*39:01 |
| 6117 | WT1 | ENSG00000184937 | TGTAGACRY | 2674 | HLA-C*16:01 |
| 6118 | WT1 | ENSG00000184937 | TGTAGACRY | 2674 | HLA-C*16:02 |
| 6119 | WT1 | ENSG00000184937 | TPILCGAQY | 2675 | HLA-B*35:01 |
| 6120 | WT1 | ENSG00000184937 | TPILCGAQY | 2675 | HLA-B*55:01 |
| 6121 | WT1 | ENSG00000184937 | TPSHHAAQF | 2676 | HLA-B*07:02 |
| 6122 | WT1 | ENSG00000184937 | TPSHHAAQF | 2676 | HLA-B*35:01 |
| 6123 | WT1 | ENSG00000184937 | TPSHHAAQF | 2676 | HLA-B*35:03 |
| 6124 | WT1 | ENSG00000184937 | TPYSSDNLYQM | 2677 | HLA-B*35:01 |
| 6125 | WT1 | ENSG00000184937 | TPYSSDNLYQM | 2677 | HLA-B*35:03 |
| 6126 | WT1 | ENSG00000184937 | TPYSSDNLYQM | 2677 | HLA-B*55:01 |
| 6127 | WT1 | ENSG00000184937 | TPYSSDNLYQM | 2677 | HLA-B*56:01 |
| 6128 | WT1 | ENSG00000184937 | TPYSSDNLY | 2678 | HLA-A*26:01 |
| 6129 | WT1 | ENSG00000184937 | TPYSSDNLY | 2678 | HLA-B*15:01 |
| 6130 | WT1 | ENSG00000184937 | TPYSSDNLY | 2678 | HLA-B*15:03 |
| 6131 | WT1 | ENSG00000184937 | TPYSSDNLY | 2678 | HLA-B*35:01 |
| 6132 | WT1 | ENSG00000184937 | TPYSSDNLY | 2678 | HLA-B*35:03 |
| 6133 | WT1 | ENSG00000184937 | TPYSSDNLY | 2678 | HLA-B*55:01 |
| 6134 | WT1 | ENSG00000184937 | TPYSSDNLY | 2678 | HLA-C*03:03 |
| 6135 | WT1 | ENSG00000184937 | TPYSSDNLY | 2678 | HLA-C*07:04 |
| 6136 | WT1 | ENSG00000184937 | TPYSSDNL | 2679 | HLA-B*07:02 |
| 6137 | WT1 | ENSG00000184937 | TPYSSDNL | 2679 | HLA-B*35:03 |
| 6138 | WT1 | ENSG00000184937 | TPYSSDNL | 2679 | HLA-B*39:01 |
| 6139 | WT1 | ENSG00000184937 | TPYSSDNL | 2679 | HLA-B*51:01 |
| 6140 | WT1 | ENSG00000184937 | TPYSSDNL | 2679 | HLA-C*07:02 |
| 6141 | WT1 | ENSG00000184937 | TSQLECMTW | 2680 | HLA-B*57:01 |
| 6142 | WT1 | ENSG00000184937 | TSQLECMTW | 2680 | HLA-B*58:01 |
| 6143 | WT1 | ENSG00000184937 | TTPILCGAQY | 2681 | HLA-A*26:01 |
| 6144 | WT1 | ENSG00000184937 | TVTFDGTPSY | 2682 | HLA-A*01:01 |
| 6145 | WT1 | ENSG00000184937 | TVTFDGTPSY | 2682 | HLA-A*25:01 |
| 6146 | WT1 | ENSG00000184937 | TVTFDGTPSY | 2682 | HLA-A*26:01 |
| 6147 | WT1 | ENSG00000184937 | TVTFDGTPSY | 2682 | HLA-A*30:02 |
| 6148 | WT1 | ENSG00000184937 | TVTFDGTPSY | 2682 | HLA-B*15:01 |
| 6149 | WT1 | ENSG00000184937 | TVTFDGTPSY | 2682 | HLA-B*39:01 |
| 6150 | WT1 | ENSG00000184937 | VAAGSSSSVKW | 2683 | HLA-A*25:01 |
| 6151 | WT1 | ENSG00000184937 | VAAGSSSSVKW | 2683 | HLA-A*32:01 |
| 6152 | WT1 | ENSG00000184937 | VAAGSSSSVKW | 2683 | HLA-A*33:01 |
| 6153 | WT1 | ENSG00000184937 | VAAGSSSSVKW | 2683 | HLA-B*57:01 |
| 6154 | WT1 | ENSG00000184937 | VAAGSSSSVKW | 2683 | HLA-B*58:01 |
| 6155 | WT1 | ENSG00000184937 | VAAGSSSSVKW | 2683 | HLA-C*02:02 |
| 6156 | WT1 | ENSG00000184937 | VAAGSSSSVKW | 2683 | HLA-C*16:02 |
| 6157 | WT1 | ENSG00000184937 | VAAGSSSSVKW | 2683 | HLA-C*16:04 |
| 6158 | WT1 | ENSG00000184937 | VAAGSSSSVK | 2684 | HLA-A*03:02 |
| 6159 | WT1 | ENSG00000184937 | VAAGSSSSV | 2685 | HLA-A*02:03 |
| 6160 | WT1 | ENSG00000184937 | VAAGSSSSV | 2685 | HLA-B*13:02 |
| 6161 | WT1 | ENSG00000184937 | VAAGSSSSV | 2685 | HLA-B*15:03 |
| 6162 | WT1 | ENSG00000184937 | VAAGSSSSV | 2685 | HLA-B*39:01 |
| 6163 | WT1 | ENSG00000184937 | VAAGSSSSV | 2685 | HLA-B*46:01 |
| 6164 | WT1 | ENSG00000184937 | VAAGSSSSV | 2685 | HLA-B*51:01 |
| 6165 | WT1 | ENSG00000184937 | VAAGSSSSV | 2685 | HLA-B*56:01 |
| 6166 | WT1 | ENSG00000184937 | VAAGSSSSV | 2685 | HLA-C*01:02 |
| 6167 | WT1 | ENSG00000184937 | VAAGSSSSV | 2685 | HLA-C*02:02 |
| 6168 | WT1 | ENSG00000184937 | VAAGSSSSV | 2685 | HLA-C*03:03 |
| 6169 | WT1 | ENSG00000184937 | VAAGSSSSV | 2685 | HLA-C*03:04 |
| 6170 | WT1 | ENSG00000184937 | VAAGSSSSV | 2685 | HLA-C*05:01 |
| 6171 | WT1 | ENSG00000184937 | VAAGSSSSV | 2685 | HLA-C*12:03 |
| 6172 | WT1 | ENSG00000184937 | VAAGSSSSV | 2685 | HLA-C*14:02 |
| 6173 | WT1 | ENSG00000184937 | VAAGSSSSV | 2685 | HLA-C*16:02 |
| 6174 | WT1 | ENSG00000184937 | VAAGSSSSV | 2685 | HLA-C*16:04 |
| 6175 | WT1 | ENSG00000184937 | VAPTLVRSA | 2686 | HLA-B*46:01 |
| 6176 | WT1 | ENSG00000184937 | VAPTLVRSA | 2686 | HLA-C*01:02 |
| 6177 | WT1 | ENSG00000184937 | VAPTLVRSA | 2686 | HLA-C*16:01 |
| 6178 | WT1 | ENSG00000184937 | VLDFAPPGA | 2687 | HLA-A*02:01 |
| 6179 | WT1 | ENSG00000184937 | VLDFAPPGA | 2687 | HLA-A*02:07 |
| 6180 | WT1 | ENSG00000184937 | VPGVAPTLV | 2688 | HLA-B*51:01 |
| 6181 | WT1 | ENSG00000184937 | VPGVAPTLV | 2688 | HLA-B*56:01 |
| 6182 | WT1 | ENSG00000184937 | VPGVAPTLV | 2688 | HLA-C*04:01 |
| 6183 | WT1 | ENSG00000184937 | VPGVAPTL | 2689 | HLA-A*23:01 |
| 6184 | WT1 | ENSG00000184937 | VPGVAPTL | 2689 | HLA-B*07:02 |
| 6185 | WT1 | ENSG00000184937 | VPGVAPTL | 2689 | HLA-B*08:01 |
| 6186 | WT1 | ENSG00000184937 | VPGVAPTL | 2689 | HLA-B*35:01 |
| 6187 | WT1 | ENSG00000184937 | VPGVAPTL | 2689 | HLA-B*35:03 |
| 6188 | WT1 | ENSG00000184937 | VPGVAPTL | 2689 | HLA-B*51:01 |
| 6189 | WT1 | ENSG00000184937 | VPGVAPTL | 2689 | HLA-B*55:01 |
| 6190 | WT1 | ENSG00000184937 | VPGVAPTL | 2689 | HLA-B*56:01 |
| 6191 | WT1 | ENSG00000184937 | VPGVAPTL | 2689 | HLA-C*05:01 |
| 6192 | WT1 | ENSG00000184937 | VPGVAPTL | 2689 | HLA-C*07:02 |
| 6193 | WT1 | ENSG00000184937 | VPGVAPTL | 2689 | HLA-C*14:02 |
| 6194 | WT1 | ENSG00000184937 | VPPPVYGC | 2690 | HLA-B*51:01 |
| 6195 | WT1 | ENSG00000184937 | VTFDGTPSYG | 2691 | HLA-C*12:03 |
| 6196 | WT1 | ENSG00000184937 | VTFDGTPSY | 2692 | HLA-A*01:01 |
| 6197 | WT1 | ENSG00000184937 | VTFDGTPSY | 2692 | HLA-A*03:01 |
| 6198 | WT1 | ENSG00000184937 | VTFDGTPSY | 2692 | HLA-A*03:02 |
| 6199 | WT1 | ENSG00000184937 | VTFDGTPSY | 2692 | HLA-A*11:01 |
| 6200 | WT1 | ENSG00000184937 | VTFDGTPSY | 2692 | HLA-A*23:01 |
| 6201 | WT1 | ENSG00000184937 | VTFDGTPSY | 2692 | HLA-A*25:01 |
| 6202 | WT1 | ENSG00000184937 | VTFDGTPSY | 2692 | HLA-A*26:01 |
| 6203 | WT1 | ENSG00000184937 | VTFDGTPSY | 2692 | HLA-A*29:02 |
| 6204 | WT1 | ENSG00000184937 | VTFDGTPSY | 2692 | HLA-A*30:02 |
| 6205 | WT1 | ENSG00000184937 | VTFDGTPSY | 2692 | HLA-A*32:01 |
| 6206 | WT1 | ENSG00000184937 | VTFDGTPSY | 2692 | HLA-B*13:02 |
| 6207 | WT1 | ENSG00000184937 | VTFDGTPSY | 2692 | HLA-B*15:01 |
| 6208 | WT1 | ENSG00000184937 | VTFDGTPSY | 2692 | HLA-B*15:03 |
| 6209 | WT1 | ENSG00000184937 | VTFDGTPSY | 2692 | HLA-B*18:01 |
| 6210 | WT1 | ENSG00000184937 | VTFDGTPSY | 2692 | HLA-B*27:02 |
| 6211 | WT1 | ENSG00000184937 | VTFDGTPSY | 2692 | HLA-B*27:05 |
| 6212 | WT1 | ENSG00000184937 | VTFDGTPSY | 2692 | HLA-B*35:01 |
| 6213 | WT1 | ENSG00000184937 | VTFDGTPSY | 2692 | HLA-B*39:01 |
| 6214 | WT1 | ENSG00000184937 | VTFDGTPSY | 2692 | HLA-B*44:03 |
| 6215 | WT1 | ENSG00000184937 | VTFDGTPSY | 2692 | HLA-B*46:01 |
| 6216 | WT1 | ENSG00000184937 | VTFDGTPSY | 2692 | HLA-B*57:01 |
| 6217 | WT1 | ENSG00000184937 | VTFDGTPSY | 2692 | HLA-B*58:01 |
| 6218 | WT1 | ENSG00000184937 | VTFDGTPSY | 2692 | HLA-C*01:02 |
| 6219 | WT1 | ENSG00000184937 | VTFDGTPSY | 2692 | HLA-C*02:02 |
| 6220 | WT1 | ENSG00000184937 | VTFDGTPSY | 2692 | HLA-C*03:03 |
| 6221 | WT1 | ENSG00000184937 | VTFDGTPSY | 2692 | HLA-C*03:04 |
| 6222 | WT1 | ENSG00000184937 | VTFDGTPSY | 2692 | HLA-C*05:01 |
| 6223 | WT1 | ENSG00000184937 | VTFDGTPSY | 2692 | HLA-C*07:04 |
| 6224 | WT1 | ENSG00000184937 | VTFDGTPSY | 2692 | HLA-C*07:06 |
| 6225 | WT1 | ENSG00000184937 | VTFDGTPSY | 2692 | HLA-C*12:03 |
| 6226 | WT1 | ENSG00000184937 | VTFDGTPSY | 2692 | HLA-C*14:02 |
| 6227 | WT1 | ENSG00000184937 | VTFDGTPSY | 2692 | HLA-C*16:01 |
| 6228 | WT1 | ENSG00000184937 | VTFDGTPSY | 2692 | HLA-C*16:02 |
| 6229 | WT1 | ENSG00000184937 | VTFDGTPSY | 2692 | HLA-C*16:04 |
| 6230 | WT1 | ENSG00000184937 | YESDNHTTPIL | 2693 | HLA-A*30:01 |
| 6231 | WT1 | ENSG00000184937 | YESDNHTTPIL | 2693 | HLA-B*40:01 |
| 6232 | WT1 | ENSG00000184937 | YESDNHTTPIL | 2693 | HLA-B*40:02 |
| 6233 | WT1 | ENSG00000184937 | YESDNHTTPIL | 2693 | HLA-C*16:04 |
| 6234 | WT1 | ENSG00000184937 | YESDNHTTPI | 2694 | HLA-B*40:01 |
| 6235 | WT1 | ENSG00000184937 | YESDNHTTPI | 2694 | HLA-B*49:01 |
| 6236 | WT1 | ENSG00000184937 | YESDNHTTP | 2695 | HLA-B*49:01 |
| 6237 | WT1 | ENSG00000184937 | YESDNHTTP | 2695 | HLA-C*16:04 |
| 6238 | WT1 | ENSG00000184937 | YGPFGPPPPS | 2696 | HLA-C*04:01 |
| 6239 | WT1 | ENSG00000184937 | YGPFGPPPPS | 2696 | HLA-C*06:02 |
| 6240 | WT1 | ENSG00000184937 | YGPFGPPPPS | 2696 | HLA-C*07:02 |
| 6241 | WT1 | ENSG00000184937 | YGPFGPPPP | 2697 | HLA-B*54:01 |
| 6242 | WT1 | ENSG00000184937 | YGPFGPPPP | 2697 | HLA-C*04:01 |
| 6243 | WT1 | ENSG00000184937 | YQMTSQLECM | 2698 | HLA-C*07:04 |
| 6244 | WT1 | ENSG00000184937 | YQMTSQLEC | 2699 | HLA-B*13:02 |
| 6245 | WT1 | ENSG00000184937 | YQMTSQLEC | 2699 | HLA-B*15:01 |
| 6246 | WT1 | ENSG00000184937 | YQMTSQLEC | 2699 | HLA-B*39:01 |
| 6247 | WT1 | ENSG00000184937 | YQMTSQLEC | 2699 | HLA-C*03:03 |
| 6248 | WT1 | ENSG00000184937 | YQMTSQLEC | 2699 | HLA-C*03:04 |
| 6249 | WT1 | ENSG00000184937 | YQMTSQLEC | 2699 | HLA-C*16:02 |
| 6250 | WT1 | ENSG00000184937 | YSSDNLYQM | 2700 | HLA-A*01:01 |
| 6251 | WT1 | ENSG00000184937 | YSSDNLYQM | 2700 | HLA-A*02:01 |
| 6252 | WT1 | ENSG00000184937 | YSSDNLYQM | 2700 | HLA-A*26:01 |
| 6253 | WT1 | ENSG00000184937 | YSSDNLYQM | 2700 | HLA-A*68:02 |
| 6254 | WT1 | ENSG00000184937 | YSSDNLYQM | 2700 | HLA-B*35:01 |
| 6255 | WT1 | ENSG00000184937 | YSSDNLYQM | 2700 | HLA-B*35:03 |
| 6256 | WT1 | ENSG00000184937 | YSSDNLYQM | 2700 | HLA-B*39:01 |
| 6257 | WT1 | ENSG00000184937 | YSSDNLYQM | 2700 | HLA-B*46:01 |
| 6258 | WT1 | ENSG00000184937 | YSSDNLYQM | 2700 | HLA-B*58:01 |
| 6259 | WT1 | ENSG00000184937 | YSSDNLYQM | 2700 | HLA-C*02:02 |
| 6260 | WT1 | ENSG00000184937 | YSSDNLYQM | 2700 | HLA-C*03:03 |
| 6261 | WT1 | ENSG00000184937 | YSSDNLYQM | 2700 | HLA-C*03:04 |
| 6262 | WT1 | ENSG00000184937 | YSSDNLYQM | 2700 | HLA-C*07:04 |
| 6263 | WT1 | ENSG00000184937 | YSSDNLYQM | 2700 | HLA-C*12:03 |
| 6264 | WT1 | ENSG00000184937 | YSSDNLYQM | 2700 | HLA-C*16:02 |
| 6265 | WT1 | ENSG00000184937 | YSVPPPVY | 2701 | HLA-A*01:01 |
| 6266 | WT1 | ENSG00000184937 | YSVPPPVY | 2701 | HLA-B*15:01 |
| 6267 | WT1 | ENSG00000184937 | YSVPPPVY | 2701 | HLA-B*15:03 |
| 6268 | WT1 | ENSG00000184937 | YSVPPPVY | 2701 | HLA-B*18:01 |
| 6269 | WT1 | ENSG00000184937 | YSVPPPVY | 2701 | HLA-B*39:01 |
| 6270 | WT1 | ENSG00000184937 | YSVPPPVY | 2701 | HLA-B*46:01 |
| 6271 | WT1 | ENSG00000184937 | YSVPPPVY | 2701 | HLA-B*57:01 |
| 6272 | WT1 | ENSG00000184937 | YSVPPPVY | 2701 | HLA-B*58:01 |
| 6273 | WT1 | ENSG00000184937 | YSVPPPVY | 2701 | HLA-C*02:02 |
| 6274 | WT1 | ENSG00000184937 | YSVPPPVY | 2701 | HLA-C*03:04 |
| 6275 | WT1 | ENSG00000184937 | YSVPPPVY | 2701 | HLA-C*12:03 |
| 6276 | WT1 | ENSG00000184937 | YSVPPPVY | 2701 | HLA-C*16:01 |
| 6277 | WT1 | ENSG00000184937 | YSVPPPVY | 2701 | HLA-C*16:02 |
| 6278 | KKLC-1 | ENSG00000204019 | ALALVRPSSS | 2702 | HLA-A*02:03 |
| 6279 | KKLC-1 | ENSG00000204019 | ALALVRPSS | 2703 | HLA-A*32:01 |
| 6280 | KKLC-1 | ENSG00000204019 | ALALVRPSS | 2703 | HLA-C*16:01 |
| 6281 | KKLC-1 | ENSG00000204019 | ALIVFWKY | 2704 | HLA-A*29:02 |
| 6282 | KKLC-1 | ENSG00000204019 | ALVRPSSSGL | 2705 | HLA-A*02:03 |
| 6283 | KKLC-1 | ENSG00000204019 | AVYDLSRDIL | 2706 | HLA-A*25:01 |
| 6284 | KKLC-1 | ENSG00000204019 | AVYDLSRDIL | 2706 | HLA-B*07:02 |
| 6285 | KKLC-1 | ENSG00000204019 | AVYDLSRDIL | 2706 | HLA-C*12:03 |
| 6286 | KKLC-1 | ENSG00000204019 | AVYDLSRDI | 2707 | HLA-B*13:02 |
| 6287 | KKLC-1 | ENSG00000204019 | CALIVFWKY | 2708 | HLA-A*29:02 |
| 6288 | KKLC-1 | ENSG00000204019 | CALIVFWKY | 2708 | HLA-B*57:01 |
| 6289 | KKLC-1 | ENSG00000204019 | DLSRDILNNF | 2709 | HLA-A*25:01 |
| 6290 | KKLC-1 | ENSG00000204019 | DNNLAVYDLSR | 2710 | HLA-A*33:01 |
| 6291 | KKLC-1 | ENSG00000204019 | DNNLAVYDLSR | 2710 | HLA-B*27:02 |
| 6292 | KKLC-1 | ENSG00000204019 | EHTLLSKGF | 2711 | HLA-B*38:01 |
| 6293 | KKLC-1 | ENSG00000204019 | EHTLLSKGF | 2711 | HLA-B*44:02 |
| 6294 | KKLC-1 | ENSG00000204019 | EHTLLSKGF | 2711 | HLA-C*07:01 |
| 6295 | KKLC-1 | ENSG00000204019 | ELEHTLLSKGF | 2712 | HLA-C*07:01 |
| 6296 | KKLC-1 | ENSG00000204019 | ELEHTLLSK | 2713 | HLA-A*01:01 |
| 6297 | KKLC-1 | ENSG00000204019 | ELEHTLLSK | 2713 | HLA-A*33:01 |
| 6298 | KKLC-1 | ENSG00000204019 | EMSSNSTAL | 2714 | HLA-A*02:03 |
| 6299 | KKLC-1 | ENSG00000204019 | EMSSNSTAL | 2714 | HLA-A*25:01 |
| 6300 | KKLC-1 | ENSG00000204019 | EMSSNSTAL | 2714 | HLA-A*26:01 |
| 6301 | KKLC-1 | ENSG00000204019 | EMSSNSTAL | 2714 | HLA-A*32:01 |
| 6302 | KKLC-1 | ENSG00000204019 | EMSSNSTAL | 2714 | HLA-A*33:03 |
| 6303 | KKLC-1 | ENSG00000204019 | EMSSNSTAL | 2714 | HLA-B*07:02 |
| 6304 | KKLC-1 | ENSG00000204019 | EMSSNSTAL | 2714 | HLA-B*08:01 |
| 6305 | KKLC-1 | ENSG00000204019 | EMSSNSTAL | 2714 | HLA-B*27:05 |
| 6306 | KKLC-1 | ENSG00000204019 | EMSSNSTAL | 2714 | HLA-B*35:03 |
| 6307 | KKLC-1 | ENSG00000204019 | EMSSNSTAL | 2714 | HLA-B*38:01 |
| 6308 | KKLC-1 | ENSG00000204019 | EMSSNSTAL | 2714 | HLA-B*39:01 |
| 6309 | KKLC-1 | ENSG00000204019 | EMSSNSTAL | 2714 | HLA-B*55:01 |
| 6310 | KKLC-1 | ENSG00000204019 | EMSSNSTAL | 2714 | HLA-C*01:02 |
| 6311 | KKLC-1 | ENSG00000204019 | EMSSNSTAL | 2714 | HLA-C*07:02 |
| 6312 | KKLC-1 | ENSG00000204019 | EMSSNSTAL | 2714 | HLA-C*07:04 |
| 6313 | KKLC-1 | ENSG00000204019 | EMSSNSTAL | 2714 | HLA-C*I4:02 |
| 6314 | KKLC-1 | ENSG00000204019 | FPHSIARQK | 2715 | HLA-B*54:01 |
| 6315 | KKLC-1 | ENSG00000204019 | FQRNTGEM | 2716 | HLA-B*07:02 |
| 6316 | KKLC-1 | ENSG00000204019 | FQRNTGEM | 2716 | HLA-B*15:01 |
| 6317 | KKLC-1 | ENSG00000204019 | FQRNTGEM | 2716 | HLA-B*15:03 |
| 6318 | KKLC-1 | ENSG00000204019 | FQRNTGEM | 2716 | HLA-B*40:02 |
| 6319 | KKLC-1 | ENSG00000204019 | FQRNTGEM | 2716 | HLA-B*46:01 |
| 6320 | KKLC-1 | ENSG00000204019 | FQRNTGEM | 2716 | HLA-C*03:03 |
| 6321 | KKLC-1 | ENSG00000204019 | FQRNTGEM | 2716 | HLA-C*03:04 |
| 6322 | KKLC-1 | ENSG00000204019 | FQRNTGEM | 2716 | HLA-C*05:01 |
| 6323 | KKLC-1 | ENSG00000204019 | FQRNTGEM | 2716 | HLA-C*07:04 |
| 6324 | KKLC-1 | ENSG00000204019 | FQRNTGEM | 2716 | HLA-C*12:03 |
| 6325 | KKLC-1 | ENSG00000204019 | FQRNTGEM | 2716 | HLA-C*14:02 |
| 6326 | KKLC-1 | ENSG00000204019 | FQRNTGEM | 2716 | HLA-C*16:01 |
| 6327 | KKLC-1 | ENSG00000204019 | FYLLLASSIL | 2717 | HLA-A*24:02 |
| 6328 | KKLC-1 | ENSG00000204019 | FYLLLASSI | 2718 | HLA-A*23:01 |
| 6329 | KKLC-1 | ENSG00000204019 | FYLLLASSI | 2718 | HLA-A*24:02 |
| 6330 | KKLC-1 | ENSG00000204019 | FYLLLASSI | 2718 | HLA-C*14:02 |
| 6331 | KKLC-1 | ENSG00000204019 | GASPHRKST | 2719 | HLA-C*16:02 |
| 6332 | KKLC-1 | ENSG00000204019 | GEMSSNSTALA | 2720 | HLA-A*30:01 |
| 6333 | KKLC-1 | ENSG00000204019 | GEMSSNSTALA | 2720 | HLA-B*40:01 |
| 6334 | KKLC-1 | ENSG00000204019 | GEMSSNSTALA | 2720 | HLA-B*49:01 |
| 6335 | KKLC-1 | ENSG00000204019 | GEMSSNSTAL | 2721 | HLA-A*30:01 |
| 6336 | KKLC-1 | ENSG00000204019 | GEMSSNSTAL | 2721 | HLA-B*15:01 |
| 6337 | KKLC-1 | ENSG00000204019 | GEMSSNSTAL | 2721 | HLA-B*15:03 |
| 6338 | KKLC-1 | ENSG00000204019 | GEMSSNSTAL | 2721 | HLA-B*27:02 |
| 6339 | KKLC-1 | ENSG00000204019 | GEMSSNSTAL | 2721 | HLA-B*27:05 |
| 6340 | KKLC-1 | ENSG00000204019 | GEMSSNSTAL | 2721 | HLA-B*38:01 |
| 6341 | KKLC-1 | ENSG00000204019 | GEMSSNSTAL | 2721 | HLA-B*39:01 |
| 6342 | KKLC-1 | ENSG00000204019 | GEMSSNSTAL | 2721 | HLA-B*40:01 |
| 6343 | KKLC-1 | ENSG00000204019 | GEMSSNSTAL | 2721 | HLA-B*40:02 |
| 6344 | KKLC-1 | ENSG00000204019 | GEMSSNSTAL | 2721 | HLA-B*44:02 |
| 6345 | KKLC-1 | ENSG00000204019 | GEMSSNSTAL | 2721 | HLA-B*44:03 |
| 6346 | KKLC-1 | ENSG00000204019 | GEMSSNSTAL | 2721 | HLA-B*49:01 |
| 6347 | KKLC-1 | ENSG00000204019 | GEMSSNSTAL | 2721 | HLA-C*16:04 |
| 6348 | KKLC-1 | ENSG00000204019 | GEMSSNSTA | 2722 | HLA-A*30:01 |
| 6349 | KKLC-1 | ENSG00000204019 | GEMSSNSTA | 2722 | HLA-B*40:01 |
| 6350 | KKLC-1 | ENSG00000204019 | GEMSSNSTA | 2722 | HLA-B*40:02 |
| 6351 | KKLC-1 | ENSG00000204019 | GEMSSNSTA | 2722 | HLA-B*49:01 |
| 6352 | KKLC-1 | ENSG00000204019 | GLINSNTDNNL | 2723 | HLA-A*02:01 |
| 6353 | KKLC-1 | ENSG00000204019 | GLINSNTDNNL | 2723 | HLA-A*02:04 |
| 6354 | KKLC-1 | ENSG00000204019 | GLINSNTDNNL | 2723 | HLA-B*27:05 |
| 6355 | KKLC-1 | ENSG00000204019 | HTLLSKGFR | 2724 | HLA-A*31:01 |
| 6356 | KKLC-1 | ENSG00000204019 | HTLLSKGFR | 2724 | HLA-A*33:01 |
| 6357 | KKLC-1 | ENSG00000204019 | HTLLSKGFR | 2724 | HLA-A*33:03 |
| 6358 | KKLC-1 | ENSG00000204019 | HTLLSKGF | 2725 | HLA-B*57:01 |
| 6359 | KKLC-1 | ENSG00000204019 | ILNNFPHSIAR | 2726 | HLA-A*03:01 |
| 6360 | KKLC-1 | ENSG00000204019 | ILNNFPHSIAR | 2726 | HLA-A*31:01 |
| 6361 | KKLC-1 | ENSG00000204019 | ILNNFPHSIAR | 2726 | HLA-A*33:01 |
| 6362 | KKLC-1 | ENSG00000204019 | ILNNFPHSIAR | 2726 | HLA-A*33:03 |
| 6363 | KKLC-1 | ENSG00000204019 | ILNNFPHSIA | 2727 | HLA-A*02:03 |
| 6364 | KKLC-1 | ENSG00000204019 | ILNNFPHSI | 2728 | HLA-A*02:01 |
| 6365 | KKLC-1 | ENSG00000204019 | ILNNFPHSI | 2728 | HLA-A*02:03 |
| 6366 | KKLC-1 | ENSG00000204019 | ILNNFPHSI | 2728 | HLA-A*02:04 |
| 6367 | KKLC-1 | ENSG00000204019 | ILNNFPHSI | 2728 | HLA-A*02:07 |
| 6368 | KKLC-1 | ENSG00000204019 | ILNNFPHSI | 2728 | HLA-A*23:01 |
| 6369 | KKLC-1 | ENSG00000204019 | ILNNFPHSI | 2728 | HLA-A*24:02 |
| 6370 | KKLC-1 | ENSG00000204019 | ILNNFPHSI | 2728 | HLA-A*30:01 |
| 6371 | KKLC-1 | ENSG00000204019 | ILNNFPHSI | 2728 | HLA-A*31:01 |
| 6372 | KKLC-1 | ENSG00000204019 | ILNNFPHSI | 2728 | HLA-A*32:01 |
| 6373 | KKLC-1 | ENSG00000204019 | ILNNFPHSI | 2728 | HLA-A*68:02 |
| 6374 | KKLC-1 | ENSG00000204019 | ILNNFPHSI | 2728 | HLA-B*13:02 |
| 6375 | KKLC-1 | ENSG00000204019 | ILNNFPHSI | 2728 | HLA-B*38:01 |
| 6376 | KKLC-1 | ENSG00000204019 | ILNNFPHSI | 2728 | HLA-B*51:01 |
| 6377 | KKLC-1 | ENSG00000204019 | ILNNFPHSI | 2728 | HLA-B*55:01 |
| 6378 | KKLC-1 | ENSG00000204019 | ILNNFPHSI | 2728 | HLA-C*01:02 |
| 6379 | KKLC-1 | ENSG00000204019 | ILNNFPHSI | 2728 | HLA-C*02:02 |
| 6380 | KKLC-1 | ENSG00000204019 | ILNNFPHSI | 2728 | HLA-C*06:02 |
| 6381 | KKLC-1 | ENSG00000204019 | ILNNFPHSI | 2728 | HLA-C*14:02 |
| 6382 | KKLC-1 | ENSG00000204019 | ILNNFPHSI | 2728 | HLA-C*16:01 |
| 6383 | KKLC-1 | ENSG00000204019 | ILNNFPHSI | 2728 | HLA-C*16:02 |
| 6384 | KKLC-1 | ENSG00000204019 | INSNTDNNL | 2729 | HLA-B*35:03 |
| 6385 | KKLC-1 | ENSG00000204019 | INSNTDNNL | 2729 | HLA-B*39:01 |
| 6386 | KKLC-1 | ENSG00000204019 | KLVELEHTLL | 2730 | HLA-A*02:01 |
| 6387 | KKLC-1 | ENSG00000204019 | KLVELEHTLL | 2730 | HLA-A*02:03 |
| 6388 | KKLC-1 | ENSG00000204019 | KLVELEHTLL | 2730 | HLA-A*02:04 |
| 6389 | KKLC-1 | ENSG00000204019 | KLVELEHTLL | 2730 | HLA-A*02:07 |
| 6390 | KKLC-1 | ENSG00000204019 | KLVELEHTL | 2731 | HLA-A*02:01 |
| 6391 | KKLC-1 | ENSG00000204019 | KLVELEHTL | 2731 | HLA-A*02:03 |
| 6392 | KKLC-1 | ENSG00000204019 | KLVELEHTL | 2731 | HLA-A*02:04 |
| 6393 | KKLC-1 | ENSG00000204019 | KLVELEHTL | 2731 | HLA-A*02:07 |
| 6394 | KKLC-1 | ENSG00000204019 | KLVELEHTL | 2731 | HLA-A*23:01 |
| 6395 | KKLC-1 | ENSG00000204019 | KLVELEHTL | 2731 | HLA-A*24:02 |
| 6396 | KKLC-1 | ENSG00000204019 | KLVELEHTL | 2731 | HLA-A*30:01 |
| 6397 | KKLC-1 | ENSG00000204019 | KLVELEHTL | 2731 | HLA-A*32:01 |
| 6398 | KKLC-1 | ENSG00000204019 | KLVELEHTL | 2731 | HLA-B*13:02 |
| 6399 | KKLC-1 | ENSG00000204019 | KLVELEHTL | 2731 | HLA-B*15:01 |
| 6400 | KKLC-1 | ENSG00000204019 | KLVELEHTL | 2731 | HLA-B*15:03 |
| 6401 | KKLC-1 | ENSG00000204019 | KLVELEHTL | 2731 | HLA-B*38:01 |
| 6402 | KKLC-1 | ENSG00000204019 | KLVELEHTL | 2731 | HLA-B*40:01 |
| 6403 | KKLC-1 | ENSG00000204019 | KLVELEHTL | 2731 | HLA-B*40:02 |
| 6404 | KKLC-1 | ENSG00000204019 | KLVELEHTL | 2731 | HLA-B*55:01 |
| 6405 | KKLC-1 | ENSG00000204019 | KLVELEHTL | 2731 | HLA-B*58:01 |
| 6406 | KKLC-1 | ENSG00000204019 | KLVELEHTL | 2731 | HLA-C*02:02 |
| 6407 | KKLC-1 | ENSG00000204019 | KLVELEHTL | 2731 | HLA-C*06:02 |
| 6408 | KKLC-1 | ENSG00000204019 | KLVELEHTL | 2731 | HLA-C*07:04 |
| 6409 | KKLC-1 | ENSG00000204019 | KLVELEHTL | 2731 | HLA-C*16:02 |
| 6410 | KKLC-1 | ENSG00000204019 | LASSILCAL | 2732 | HLA-A*68:02 |
| 6411 | KKLC-1 | ENSG00000204019 | LASSILCAL | 2732 | HLA-B*27:05 |
| 6412 | KKLC-1 | ENSG00000204019 | LASSILCAL | 2732 | HLA-B*35:03 |
| 6413 | KKLC-1 | ENSG00000204019 | LASSILCAL | 2732 | HLA-B*46:01 |
| 6414 | KKLC-1 | ENSG00000204019 | LASSILCAL | 2732 | HLA-C*02:02 |
| 6415 | KKLC-1 | ENSG00000204019 | LASSILCAL | 2732 | HLA-C*03:03 |
| 6416 | KKLC-1 | ENSG00000204019 | LASSILCAL | 2732 | HLA-C*03:04 |
| 6417 | KKLC-1 | ENSG00000204019 | LASSILCAL | 2732 | HLA-C*07:04 |
| 6418 | KKLC-1 | ENSG00000204019 | LASSILCAL | 2732 | HLA-C*12:03 |
| 6419 | KKLC-1 | ENSG00000204019 | LASSILCAL | 2732 | HLA-C*16:01 |
| 6420 | KKLC-1 | ENSG00000204019 | LASSILCAL | 2732 | HLA-C*16:02 |
| 6421 | KKLC-1 | ENSG00000204019 | LASSILCA | 2733 | HLA-B*54:01 |
| 6422 | KKLC-1 | ENSG00000204019 | LEHTLLSKGF | 2734 | HLA-B*44:02 |
| 6423 | KKLC-1 | ENSG00000204019 | LEHTLLSKGF | 2734 | HLA-B*44:03 |
| 6424 | KKLC-1 | ENSG00000204019 | LINSNTDNNL | 2735 | HLA-B*27:05 |
| 6425 | KKLC-1 | ENSG00000204019 | LLASSILCAL | 2736 | HLA-A*02:03 |
| 6426 | KKLC-1 | ENSG00000204019 | LLASSILCAL | 2736 | HLA-A*02:04 |
| 6427 | KKLC-1 | ENSG00000204019 | LLASSILCA | 2737 | HLA-A*02:01 |
| 6428 | KKLC-1 | ENSG00000204019 | LLASSILCA | 2737 | HLA-A*02:03 |
| 6429 | KKLC-1 | ENSG00000204019 | LLLASSILCAL | 2738 | HLA-A*02:01 |
| 6430 | KKLC-1 | ENSG00000204019 | LLLASSILCAL | 2738 | HLA-A*02:04 |
| 6431 | KKLC-1 | ENSG00000204019 | LLLASSILC | 2739 | HLA-A*02:01 |
| 6432 | KKLC-1 | ENSG00000204019 | LSRDILNNF | 2740 | HLA-A*23:01 |
| 6433 | KKLC-1 | ENSG00000204019 | LSRDILNNF | 2740 | HLA-A*24:02 |
| 6434 | KKLC-1 | ENSG00000204019 | LSRDILNNF | 2740 | HLA-A*32:01 |
| 6435 | KKLC-1 | ENSG00000204019 | LSRDILNNF | 2740 | HLA-A*33:01 |
| 6436 | KKLC-1 | ENSG00000204019 | LSRDILNNF | 2740 | HLA-A*33:03 |
| 6437 | KKLC-1 | ENSG00000204019 | LSRDILNNF | 2740 | HLA-B*46:01 |
| 6438 | KKLC-1 | ENSG00000204019 | LSRDILNNF | 2740 | HLA-B*57:01 |
| 6439 | KKLC-1 | ENSG00000204019 | LSRDILNNF | 2740 | HLA-B*58:01 |
| 6440 | KKLC-1 | ENSG00000204019 | LSRDILNNF | 2740 | HLA-C*02:02 |
| 6441 | KKLC-1 | ENSG00000204019 | LSRDILNNF | 2740 | HLA-C*16:01 |
| 6442 | KKLC-1 | ENSG00000204019 | LVELEHTLL | 2741 | HLA-A*01:01 |
| 6443 | KKLC-1 | ENSG00000204019 | LVELEHTLL | 2741 | HLA-A*02:07 |
| 6444 | KKLC-1 | ENSG00000204019 | LVELEHTLL | 2741 | HLA-B*38:01 |
| 6445 | KKLC-1 | ENSG00000204019 | LVELEHTLL | 2741 | HLA-C*05:01 |
| 6446 | KKLC-1 | ENSG00000204019 | LVELEHTLL | 2741 | HLA-C*07:01 |
| 6447 | KKLC-1 | ENSG00000204019 | LVELEHTL | 2742 | HLA-C*05:01 |
| 6448 | KKLC-1 | ENSG00000204019 | LVELEHTL | 2742 | HLA-C*07:04 |
| 6449 | KKLC-1 | ENSG00000204019 | LVNLSMVENK | 2743 | HLA-A*03:02 |
| 6450 | KKLC-1 | ENSG00000204019 | LVNLSMVENK | 2743 | HLA-B*27:02 |
| 6451 | KKLC-1 | ENSG00000204019 | LVRPSSSGLI | 2744 | HLA-C*07:02 |
| 6452 | KKLC-1 | ENSG00000204019 | LVRPSSSGL | 2745 | HLA-A*02:03 |
| 6453 | KKLC-1 | ENSG00000204019 | LVRPSSSGL | 2745 | HLA-A*32:01 |
| 6454 | KKLC-1 | ENSG00000204019 | LVRPSSSGL | 2745 | HLA-B*07:02 |
| 6455 | KKLC-1 | ENSG00000204019 | LVRPSSSGL | 2745 | HLA-B*08:01 |
| 6456 | KKLC-1 | ENSG00000204019 | LVRPSSSGL | 2745 | HLA-B*15:01 |
| 6457 | KKLC-1 | ENSG00000204019 | LVRPSSSGL | 2745 | HLA-B*40:02 |
| 6458 | KKLC-1 | ENSG00000204019 | LVRPSSSGL | 2745 | HLA-B*46:01 |
| 6459 | KKLC-1 | ENSG00000204019 | LVRPSSSGL | 2745 | HLA-C*01:02 |
| 6460 | KKLC-1 | ENSG00000204019 | LVRPSSSGL | 2745 | HLA-C*03:03 |
| 6461 | KKLC-1 | ENSG00000204019 | LVRPSSSGL | 2745 | HLA-C*03:04 |
| 6462 | KKLC-1 | ENSG00000204019 | LVRPSSSGL | 2745 | HLA-C*07:02 |
| 6463 | KKLC-1 | ENSG00000204019 | LVRPSSSGL | 2745 | HLA-C*07:04 |
| 6464 | KKLC-1 | ENSG00000204019 | LVRPSSSGL | 2745 | HLA-C*14:02 |
| 6465 | KKLC-1 | ENSG00000204019 | MSSNSTAL | 2746 | HLA-C*01:02 |
| 6466 | KKLC-1 | ENSG00000204019 | MSSNSTAL | 2746 | HLA-C*03:03 |
| 6467 | KKLC-1 | ENSG00000204019 | MSSNSTAL | 2746 | HLA-C*03:04 |
| 6468 | KKLC-1 | ENSG00000204019 | MSSNSTAL | 2746 | HLA-C*05:01 |
| 6469 | KKLC-1 | ENSG00000204019 | MSSNSTAL | 2746 | HLA-C*07:04 |
| 6470 | KKLC-1 | ENSG00000204019 | MSSNSTAL | 2746 | HLA-C*14:02 |
| 6471 | KKLC-1 | ENSG00000204019 | MSSNSTAL | 2746 | HLA-C*16:01 |
| 6472 | KKLC-1 | ENSG00000204019 | MVENKLVEL | 2747 | HLA-A*02:07 |
| 6473 | KKLC-1 | ENSG00000204019 | MVENKLVEL | 2747 | HLA-A*68:02 |
| 6474 | KKLC-1 | ENSG00000204019 | MVENKLVEL | 2747 | HLA-B*08:01 |
| 6475 | KKLC-1 | ENSG00000204019 | MVENKLVEL | 2747 | HLA-B*35:03 |
| 6476 | KKLC-1 | ENSG00000204019 | MVENKLVEL | 2747 | HLA-B*38:01 |
| 6477 | KKLC-1 | ENSG00000204019 | MVENKLVEL | 2747 | HLA-B*55:01 |
| 6478 | KKLC-1 | ENSG00000204019 | MVENKLVEL | 2747 | HLA-C*01:02 |
| 6479 | KKLC-1 | ENSG00000204019 | MVENKLVEL | 2747 | HLA-C*03:04 |
| 6480 | KKLC-1 | ENSG00000204019 | MVENKLVEL | 2747 | HLA-C*05:01 |
| 6481 | KKLC-1 | ENSG00000204019 | MVENKLVEL | 2747 | HLA-C*07:04 |
| 6482 | KKLC-1 | ENSG00000204019 | NLSMVENKL | 2748 | HLA-A*02:04 |
| 6483 | KKLC-1 | ENSG00000204019 | NNFPHSIAR | 284 | HLA-A*31:01 |
| 6484 | KKLC-1 | ENSG00000204019 | NNFPHSIAR | 284 | HLA-A*33:01 |
| 6485 | KKLC-1 | ENSG00000204019 | NNFPHSIAR | 284 | HLA-A*33:03 |
| 6486 | KKLC-1 | ENSG00000204019 | NNFPHSIAR | 284 | HLA-A*68:01 |
| 6487 | KKLC-1 | ENSG00000204019 | NNFPHSIAR | 284 | HLA-A*68:02 |
| 6488 | KKLC-1 | ENSG00000204019 | NNFPHSIAR | 284 | HLA-C*07:06 |
| 6489 | KKLC-1 | ENSG00000204019 | NSNTDNNLAVY | 2749 | HLA-A*01:01 |
| 6490 | KKLC-1 | ENSG00000204019 | NSNTDNNLAVY | 2749 | HLA-A*30:02 |
| 6491 | KKLC-1 | ENSG00000204019 | NSTALALVR | 2750 | HLA-A*29:02 |
| 6492 | KKLC-1 | ENSG00000204019 | NSTALALVR | 2750 | HLA-A*33:01 |
| 6493 | KKLC-1 | ENSG00000204019 | NSTALALVR | 2750 | HLA-A*33:03 |
| 6494 | KKLC-1 | ENSG00000204019 | NSTALALVR | 2750 | HLA-A*68:01 |
| 6495 | KKLC-1 | ENSG00000204019 | NSTALALVR | 2750 | HLA-B*27:02 |
| 6496 | KKLC-1 | ENSG00000204019 | NSTALALVR | 2750 | HLA-C*07:06 |
| 6497 | KKLC-1 | ENSG00000204019 | NTDNNLAVYDL | 2751 | HLA-B*27:05 |
| 6498 | KKLC-1 | ENSG00000204019 | NTDNNLAVYDL | 2751 | HLA-B*39:01 |
| 6499 | KKLC-1 | ENSG00000204019 | NTDNNLAVYDL | 2751 | HLA-C*04:01 |
| 6500 | KKLC-1 | ENSG00000204019 | NTDNNLAVY | 23 | HLA-A*01:01 |
| 6501 | KKLC-1 | ENSG00000204019 | NTDNNLAVY | 23 | HLA-A*25:01 |
| 6502 | KKLC-1 | ENSG00000204019 | NTDNNLAVY | 23 | HLA-A*26:01 |
| 6503 | KKLC-1 | ENSG00000204019 | NTDNNLAVY | 23 | HLA-A*29:02 |
| 6504 | KKLC-1 | ENSG00000204019 | NTDNNLAVY | 23 | HLA-A*30:02 |
| 6505 | KKLC-1 | ENSG00000204019 | NTDNNLAVY | 23 | HLA-A*32:01 |
| 6506 | KKLC-1 | ENSG00000204019 | NTDNNLAVY | 23 | HLA-B*18:01 |
| 6507 | KKLC-1 | ENSG00000204019 | NTDNNLAVY | 23 | HLA-B*27:05 |
| 6508 | KKLC-1 | ENSG00000204019 | NTDNNLAVY | 23 | HLA-B*35:01 |
| 6509 | KKLC-1 | ENSG00000204019 | NTDNNLAVY | 23 | HLA-B*39:01 |
| 6510 | KKLC-1 | ENSG00000204019 | NTDNNLAVY | 23 | HLA-B*55:01 |
| 6511 | KKLC-1 | ENSG00000204019 | NTDNNLAVY | 23 | HLA-C*03:03 |
| 6512 | KKLC-1 | ENSG00000204019 | NTDNNLAVY | 23 | HLA-C*04:01 |
| 6513 | KKLC-1 | ENSG00000204019 | NTDNNLAVY | 23 | HLA-C*05:01 |
| 6514 | KKLC-1 | ENSG00000204019 | NTDNNLAVY | 23 | HLA-C*07:01 |
| 6515 | KKLC-1 | ENSG00000204019 | NTDNNLAVY | 23 | HLA-C*07:04 |
| 6516 | KKLC-1 | ENSG00000204019 | NTDNNLAVY | 23 | HLA-C*07:06 |
| 6517 | KKLC-1 | ENSG00000204019 | NTDNNLAVY | 23 | HLA-C*16:02 |
| 6518 | KKLC-1 | ENSG00000204019 | NTDNNLAV | 2752 | HLA-A*01:01 |
| 6519 | KKLC-1 | ENSG00000204019 | NTDNNLAV | 2752 | HLA-C*05:01 |
| 6520 | KKLC-1 | ENSG00000204019 | RFQRNTGEM | 2753 | HLA-C*14:02 |
| 6521 | KKLC-1 | ENSG00000204019 | RILVNLSMV | 2754 | HLA-A*02:01 |
| 6522 | KKLC-1 | ENSG00000204019 | RILVNLSMV | 2754 | HLA-A*02:03 |
| 6523 | KKLC-1 | ENSG00000204019 | RILVNLSMV | 2754 | HLA-A*02:04 |
| 6524 | KKLC-1 | ENSG00000204019 | RILVNLSMV | 2754 | HLA-A*02:07 |
| 6525 | KKLC-1 | ENSG00000204019 | RPSSSGLI | 2755 | HLA-C*07:02 |
| 6526 | KKLC-1 | ENSG00000204019 | RQKRILVNL | 2756 | HLA-A*31:01 |
| 6527 | KKLC-1 | ENSG00000204019 | SMVENKLVEL | 2757 | HLA-A*02:01 |
| 6528 | KKLC-1 | ENSG00000204019 | SMVENKLVEL | 2757 | HLA-A*02:03 |
| 6529 | KKLC-1 | ENSG00000204019 | SMVENKLVEL | 2757 | HLA-A*02:04 |
| 6530 | KKLC-1 | ENSG00000204019 | SMVENKLVEL | 2757 | HLA-A*02:07 |
| 6531 | KKLC-1 | ENSG00000204019 | SMVENKLVEL | 2757 | HLA-B*46:01 |
| 6532 | KKLC-1 | ENSG00000204019 | SMVENKLVEL | 2757 | HLA-B*55:01 |
| 6533 | KKLC-1 | ENSG00000204019 | SNSTALALVR | 2758 | HLA-A*68:01 |
| 6534 | KKLC-1 | ENSG00000204019 | SNSTALALVR | 2758 | HLA-B*27:02 |
| 6535 | KKLC-1 | ENSG00000204019 | SNSTALALVR | 2758 | HLA-C*07:06 |
| 6536 | KKLC-1 | ENSG00000204019 | SNTDNNLAVY | 2759 | HLA-A*25:01 |
| 6537 | KKLC-1 | ENSG00000204019 | SNTDNNLAVY | 2759 | HLA-A*26:01 |
| 6538 | KKLC-1 | ENSG00000204019 | SNTDNNLAVY | 2759 | HLA-A*29:02 |
| 6539 | KKLC-1 | ENSG00000204019 | SNTDNNLAVY | 2759 | HLA-A*30:02 |
| 6540 | KKLC-1 | ENSG00000204019 | SNTDNNLAV | 2760 | HLA-B*13:02 |
| 6541 | KKLC-1 | ENSG00000204019 | SSNSTALALVR | 2761 | HLA-A*03:02 |
| 6542 | KKLC-1 | ENSG00000204019 | SSNSTALALVR | 2761 | HLA-A*11:01 |
| 6543 | KKLC-1 | ENSG00000204019 | SSNSTALALVR | 2761 | HLA-A*31:01 |
| 6544 | KKLC-1 | ENSG00000204019 | SSNSTALALVR | 2761 | HLA-A*33:03 |
| 6545 | KKLC-1 | ENSG00000204019 | SSNSTALALVR | 2761 | HLA-A*68:01 |
| 6546 | KKLC-1 | ENSG00000204019 | SSNSTALALVR | 2761 | HLA-C*07:06 |
| 6547 | KKLC-1 | ENSG00000204019 | SSNSTALAL | 2762 | HLA-A*03:01 |
| 6548 | KKLC-1 | ENSG00000204019 | SSNSTALAL | 2762 | HLA-B*07:02 |
| 6549 | KKLC-1 | ENSG00000204019 | SSNSTALAL | 2762 | HLA-B*13:02 |
| 6550 | KKLC-1 | ENSG00000204019 | SSNSTALAL | 2762 | HLA-B*35:03 |
| 6551 | KKLC-1 | ENSG00000204019 | SSNSTALAL | 2762 | HLA-B*46:01 |
| 6552 | KKLC-1 | ENSG00000204019 | SSNSTALAL | 2762 | HLA-B*58:01 |
| 6553 | KKLC-1 | ENSG00000204019 | SSNSTALAL | 2762 | HLA-C*01:02 |
| 6554 | KKLC-1 | ENSG00000204019 | SSNSTALAL | 2762 | HLA-C*03:03 |
| 6555 | KKLC-1 | ENSG00000204019 | SSNSTALAL | 2762 | HLA-C*03:04 |
| 6556 | KKLC-1 | ENSG00000204019 | SSNSTALAL | 2762 | HLA-C*07:02 |
| 6557 | KKLC-1 | ENSG00000204019 | SSNSTALAL | 2762 | HLA-C*07:06 |
| 6558 | KKLC-1 | ENSG00000204019 | SSNSTALAL | 2762 | HLA-C*14:02 |
| 6559 | KKLC-1 | ENSG00000204019 | SSNSTALAL | 2762 | HLA-C*16:01 |
| 6560 | KKLC-1 | ENSG00000204019 | SSNSTALAL | 2762 | HLA-C*16:02 |
| 6561 | KKLC-1 | ENSG00000204019 | SSSGLINSN | 2763 | HLA-C*12:03 |
| 6562 | KKLC-1 | ENSG00000204019 | STALALVRPSS | 2764 | HLA-C*06:02 |
| 6563 | KKLC-1 | ENSG00000204019 | STALALVRPS | 2765 | HLA-C*06:02 |
| 6564 | KKLC-1 | ENSG00000204019 | STALALVRPS | 2765 | HLA-C*07:01 |
| 6565 | KKLC-1 | ENSG00000204019 | STALALVRPS | 2765 | HLA-C*07:02 |
| 6566 | KKLC-1 | ENSG00000204019 | TALALVRPS | 2766 | HLA-A*31:01 |
| 6567 | KKLC-1 | ENSG00000204019 | TALALVRPS | 2766 | HLA-A*33:01 |
| 6568 | KKLC-1 | ENSG00000204019 | TALALVRPS | 2766 | HLA-C*06:02 |
| 6569 | KKLC-1 | ENSG00000204019 | TALALVRPS | 2766 | HLA-C*07:01 |
| 6570 | KKLC-1 | ENSG00000204019 | TALALVRPS | 2766 | HLA-C*12:03 |
| 6571 | KKLC-1 | ENSG00000204019 | TDNNLAVY | 2767 | HLA-A*30:02 |
| 6572 | KKLC-1 | ENSG00000204019 | TDNNLAVY | 2767 | HLA-B*18:01 |
| 6573 | KKLC-1 | ENSG00000204019 | TDNNLAVY | 2767 | HLA-C*04:01 |
| 6574 | KKLC-1 | ENSG00000204019 | TDNNLAVY | 2767 | HLA-C*07:01 |
| 6575 | KKLC-1 | ENSG00000204019 | TDNNLAVY | 2767 | HLA-C*07:04 |
| 6576 | KKLC-1 | ENSG00000204019 | TGEMSSNSTAL | 2768 | HLA-B*35:03 |
| 6577 | KKLC-1 | ENSG00000204019 | TGEMSSNSTAL | 2768 | HLA-B*40:01 |
| 6578 | KKLC-1 | ENSG00000204019 | TLLSKGFRGA | 2769 | HLA-A*02:03 |
| 6579 | KKLC-1 | ENSG00000204019 | VELEHTLLS | 2770 | HLA-B*49:01 |
| 6580 | KKLC-1 | ENSG00000204019 | VELEHTLL | 2771 | HLA-A*23:01 |
| 6581 | KKLC-1 | ENSG00000204019 | VELEHTLL | 2771 | HLA-A*30:01 |
| 6582 | KKLC-1 | ENSG00000204019 | VELEHTLL | 2771 | HLA-B*08:01 |
| 6583 | KKLC-1 | ENSG00000204019 | VELEHTLL | 2771 | HLA-B*18:01 |
| 6584 | KKLC-1 | ENSG00000204019 | VELEHTLL | 2771 | HLA-B*37:01 |
| 6585 | KKLC-1 | ENSG00000204019 | VELEHTLL | 2771 | HLA-B*40:01 |
| 6586 | KKLC-1 | ENSG00000204019 | VELEHTLL | 2771 | HLA-B*40:02 |
| 6587 | KKLC-1 | ENSG00000204019 | VELEHTLL | 2771 | HLA-B*49:01 |
| 6588 | KKLC-1 | ENSG00000204019 | VELEHTLL | 2771 | HLA-C*04:01 |
| 6589 | KKLC-1 | ENSG00000204019 | VELEHTLL | 2771 | HLA-C*07:01 |
| 6590 | KKLC-1 | ENSG00000204019 | VENKLVEL | 2772 | HLA-A*30:01 |
| 6591 | KKLC-1 | ENSG00000204019 | VENKLVEL | 2772 | HLA-B*18:01 |
| 6592 | KKLC-1 | ENSG00000204019 | VENKLVEL | 2772 | HLA-B*37:01 |
| 6593 | KKLC-1 | ENSG00000204019 | VENKLVEL | 2772 | HLA-B*40:01 |
| 6594 | KKLC-1 | ENSG00000204019 | VENKLVEL | 2772 | HLA-B*40:02 |
| 6595 | KKLC-1 | ENSG00000204019 | VENKLVEL | 2772 | HLA-B*49:01 |
| 6596 | KKLC-1 | ENSG00000204019 | VNLSMVENKL | 2773 | HLA-A*23:01 |
| 6597 | KKLC-1 | ENSG00000204019 | VNLSMVENK | 2774 | HLA-B*27:02 |
| 6598 | KKLC-1 | ENSG00000204019 | VRPSSSGLIN | 2775 | HLA-C*07:01 |
| 6599 | KKLC-1 | ENSG00000204019 | VRPSSSGLI | 2776 | HLA-C*06:02 |
| 6600 | KKLC-1 | ENSG00000204019 | VRPSSSGL | 2777 | HLA-C*01:02 |
| 6601 | KKLC-1 | ENSG00000204019 | VRPSSSGL | 2777 | HLA-C*07:02 |
| 6602 | KKLC-1 | ENSG00000204019 | VYDLSRDIL | 2778 | HLA-A*23:01 |
| 6603 | KKLC-1 | ENSG00000204019 | VYDLSRDIL | 2778 | HLA-A*24:02 |
| 6604 | KKLC-1 | ENSG00000204019 | VYDLSRDIL | 2778 | HLA-B*35:01 |
| 6605 | KKLC-1 | ENSG00000204019 | VYDLSRDIL | 2778 | HLA-B*35:03 |
| 6606 | KKLC-1 | ENSG00000204019 | VYDLSRDIL | 2778 | HLA-B*55:01 |
| 6607 | KKLC-1 | ENSG00000204019 | VYDLSRDIL | 2778 | HLA-C*04:01 |
| 6608 | KKLC-1 | ENSG00000204019 | VYDLSRDI | 2779 | HLA-B*35:03 |
| 6609 | KKLC-1 | ENSG00000204019 | VYDLSRDI | 2779 | HLA-B*51:01 |
| 6610 | KKLC-1 | ENSG00000204019 | VYDLSRDI | 2779 | HLA-C*04:01 |
| 6611 | KKLC-1 | ENSG00000204019 | YLLLASSI | 2780 | HLA-B*13:02 |
| 6612 | KKLC-1 | ENSG00000204019 | YLLLASSI | 2780 | HLA-B*51:01 |

1. An isolated antigen binding protein (ABP) that specifically binds to a human leukocyte antigen (HLA)-PEPTIDE target, wherein the HLA-PEPTIDE target comprises an HLA-restricted peptide complexed with an HLA Class I molecule, wherein the HLA-restricted peptide is located in the peptide binding groove of an α1/α2 heterodimer portion of the HLA Class I molecule, wherein the HLA-peptide is selected from Table A, and wherein:
a. the HLA Class I molecule is HLA subtype A*02:01 and the HLA-restricted peptide comprises the sequence LLASSILCA,
b. the HLA Class I molecule is HLA subtype A*01:01 and the HLA-restricted peptide comprises the sequence EVDPIGHLY,
c. the HLA Class I molecule is HLA subtype B*44:02 and the HLA-restricted peptide comprises the sequence GEMSSNSTAL,
d. the HLA Class I molecule is HLA subtype A*02:01 and the HLA-restricted peptide comprises the sequence GVYDGEEHSV,
e. the HLA Class I molecule is HLA subtype *01:01 and the HLA-restricted peptide comprises the sequence EVDPIGHVY, or
f. the HLA Class I molecule is HLA subtype HLA-A*01:01 and the HLA-restricted peptide comprises the sequence NTDNNLAVY.

2-14. (canceled)

15. The isolated ABP of claim 1, wherein the ABP comprises a CDR-H3 comprising a sequence set forth in any one of SEQ ID NOS: 2902-2933 and/or wherein the ABP comprises a CDR-L3 comprising a sequence set forth in any one of SEQ ID NOS: 2971-2993.

16. (canceled)

17. The isolated ABP of claim 1, wherein the ABP comprises the CDR-H3 and the CDR-L3 from the scFv designated G2-P2E07, G2-P2E03, G2-P2A11, G2-P2C06, G2-P1G01, G2-P1C02, G2-P1H01, G2-P1B12, G2-P1B06, G2-P2H10, G2-P1H10, G2-P2C11, G2-P1C09, G2-P1A10, G2-P1B10, G2-P1D07, G2-P1E05, G2-P1D03, G2-P1G12, G2-P2H11, G2-P1C03, G2-P1G07, G2-P1F12, G2-P1G03, G2-P2B08, G2-P2A10, G2-P2D04, G2-P1C06, G2-P2A09, G2-P1B08, G2-P1E03, G2-P2A03, G2-P2F01, G2-P1H11, or G2-P1D06.

18. (canceled)

19. The isolated ABP of claim 1, wherein the ABP comprises a VH sequence selected from 2781-2815 and/or wherein the ABP comprises a VL sequence selected from 2816-2850.

20-21. (canceled)

22. The isolated ABP of claim 1, wherein the ABP binds to the HLA-PEPTIDE target (i) via residues 6-9 of the restricted peptide NTDNNLAVY and via residues 157-160 of the HLA subtype allele A*0101 or (ii) via residues 3-8 of the restricted peptide NTDNNLAVY.

23-96. (canceled)

97. An isolated HLA-PEPTIDE target, wherein the HLA-PEPTIDE target comprises an HLA-restricted peptide complexed with an HLA Class I molecule, wherein the HLA-restricted peptide is located in in the peptide binding groove of an α1/α2 heterodimer portion of the HLA Class I molecule, and wherein the HLA-PEPTIDE target is selected from Table A, with the proviso that the isolated HLA-PEPTIDE target is not any one of Target nos. 6364-6369, 6386-6389, 6500, 6521-6524, or 6578 and is not an HLA-PEPTIDE target found in Table B or Table C.

98. The isolated HLA-PEPTIDE target of claim 97, wherein
a. the HLA Class I molecule is HLA subtype A*02:01 and the HLA-restricted peptide comprises the sequence LLASSILCA,
b. the HLA Class I molecule is HLA subtype A*01:01 and the HLA-restricted peptide comprises the sequence EVDPIGHLY,
c. the HLA Class I molecule is HLA subtype B*44:02 and the HLA-restricted peptide comprises the sequence GEMSSNSTAL,
d. the HLA Class I molecule is HLA subtype A*02:01 and the HLA-restricted peptide comprises the sequence GVYDGEEHSV,
e. the HLA Class I molecule is HLA subtype *01:01 and the HLA-restricted peptide comprises the sequence EVDPIGHVY, or
f. the HLA Class I molecule is HLA subtype HLA-A*01:01 and the HLA-restricted peptide comprises the sequence NTDNNLAVY.

99-110. (canceled)

111. A composition comprising an HLA-PEPTIDE target of claim 97 attached to a solid support.

112-114. (canceled)

115. A reaction mixture comprising
a. an isolated and purified α-subunit of an HLA subtype as described in Table A;
a. an isolated and purified β2-microglobin subunit of the HLA subtype;
b. an isolated and purified restricted peptide as described in Table A; and
c. a reaction buffer.

116-117. (canceled)

118. An isolated polynucleotide comprising a first nucleic acid sequence encoding an HLA-restricted peptide as defined in claim 97, operably linked to a promoter, and a second nucleic acid sequence encoding an HLA subtype as defined in claim 97, wherein the second nucleic acid is operably linked to the same or different promoter as the first nucleic acid sequence, and wherein the encoded peptide and encoded HLA subtype form an HLA/peptide complex as defined in claim 97.

119-123. (canceled)

124. A host cell comprising (a) a heterologous HLA-PEPTIDE target of claim 97 or (b) an HLA-restricted peptide as described in Table A.

125-128. (canceled)

129. A cell culture system comprising
a. a host cell of claim 124, and
b. a cell culture medium comprising a restricted peptide as described in Table A.

130-141. (canceled)

142. An isolated polynucleotide or set of polynucleotides encoding the antigen binding protein of claim 97 or an antigen-binding portion thereof.

143-145. (canceled)

146. A method of producing an antigen binding protein comprising expressing the antigen binding protein with a host cell comprising the isolated polynucleotide of claim 142 and isolating the expressed antigen binding protein.

147. (canceled)

148. A method of treating cancer in a subject, comprising administering to the subject an effective amount of the antigen binding protein of claim 97.

149. (canceled)

150. A kit comprising
(1)(a) the antigen binding protein of claim 97 or (1)(b) a pharmaceutical composition comprising the antigen binding protein of claim 97 and a pharmaceutically acceptable excipient; and
(2) instructions for use.

151-152. (canceled)

153. A composition comprising an amino acid sequence comprising a polypeptide of at least one HLA-PEPTIDE target of claim 97.

154. A virus comprising the isolated polynucleotide or set of polynucleotides of claim 142.

155-156. (canceled)

157. A method of identifying an antigen binding protein of claim 97, comprising providing at least one HLA-PEPTIDE target listed in Table A; and binding the at least one target with the antigen binding protein, thereby identifying the antigen binding protein.

158-173. (canceled)

174. A method of identifying an antigen binding protein of claim 97, comprising obtaining one or more cells comprising the antigen binding protein; activating the one or more cells with at least one HLA-PEPTIDE target listed in Table A presented on a natural or an artificial antigen presenting cell (APC); and identifying the antigen binding protein via selection of one or more cells activated by interaction with at least one HLA-PEPTIDE target listed in Table A.

175-178. (canceled)
